(19) United States
(12) Patent Application Publication
(10) Pub. No.: US 2026/0305162 A1
(43) Pub. Date: Oct. 1, 2026

(54) Light Emitting Element, Amine Compound For The Light Emitting Element, And Electronic Device Including The Light Emitting Element

(71) Applicant: Samsung Display Co., Ltd., Yongin-si (KR)

(72) Inventor: Naoya Sakamoto, Yokohama-shi (JP)

(73) Assignee: Samsung Display Co., Ltd., Yongin-si (KR)

(21) Appl. No.: 19/453,301

(22) Filed: Jan. 20, 2026

(30) Foreign Application Priority Data
Mar. 17, 2025 (JP) 2025-042779

(51) Int. Cl. H10K 85/60; C07B 59/00; C07D 307/91; C07D 333/76; C09K 11/02; H10K 50/15

(52) U.S. Cl. CPC H10K 85/636; C07B 59/002; C07D 307/91; C07D 333/76; C09K 11/02; H10K 85/633; H10K 50/15; H10K 85/615; H10K 85/626; H10K 85/6574; H10K 85/6576

(57) ABSTRACT

A light emitting element may include a first electrode, a second electrode disposed on the first electrode, an emission layer between the first electrode and the second electrode, and a hole transport region between the first electrode and the emission layer, wherein the hole transport region includes an amine compound represented by Formula 1. The variables in Formula 1 are described in detail herein. The light emitting element may exhibit high efficiency and long lifespan characteristics.

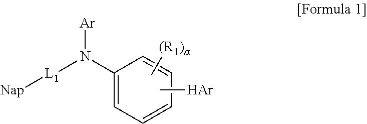

### CROSS-REFERENCE TO RELATED APPLICATIONS

This U.S. non-provisional patent application claims priority under 35 U.S.C. § 119 to Japanese Patent Application No. 2025-042779, filed on Mar. 17, 2025, the entire contents of which are hereby incorporated by reference.

### TECHNICAL FIELD

The present disclosure relates to a light emitting element, an amine compound for the light emitting element, and an electronic device including the light emitting element.

### BACKGROUND

Electronic devices include display devices. As image display devices, organic electroluminescence display devices and the like have been actively developed lately. The organic electroluminescence display devices and the like are display devices including so-called self-luminescent light emitting elements in which holes and electrons injected from a first electrode and a second electrode recombine in an emission layer, and thus a luminescent material in the emission layer emits light to accomplish display.

For application of light emitting elements to display devices, there is a demand for greater light efficiency and service life, and development of materials, for light emitting elements, capable of stably attaining such characteristics is being continuously required.

### SUMMARY

The present disclosure provides a light emitting element having improved luminous efficiency and lifespan.

The present disclosure also provides an amine compound as a material for a light emitting element having high efficiency and long lifespan.

The present disclosure also provides an electronic device having high display quality, including a light emitting element having improved luminous efficiency and lifespan.

An aspect of the present disclosure provides a light emitting element including a first electrode, a second electrode disposed on the first electrode, an emission layer disposed between the first electrode and the second electrode, and a hole transport region disposed between the first electrode and the emission layer and including an amine compound represented by Formula 1 below.

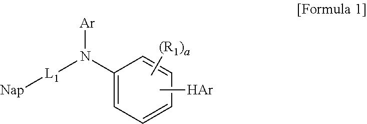

In Formula 1 above, L₁ may be a direct linkage, a substituted or unsubstituted arylene group having 6 to 30 ring-forming carbon atoms excluding a substituted or unsubstituted naphthyl group, or a substituted or unsubstituted heteroarylene group having 5 to 30 ring-forming carbon atoms, Ar may be a substituted or unsubstituted phenyl group without including an aryl group or a heteroaryl group having 16 or more ring-forming carbon atoms as a substituent, or a substituted or unsubstituted naphthyl group,
a may be an integer of 0 to 4, and R₁ may be a hydrogen atom, a deuterium atom, a halogen atom, a substituted or unsubstituted alkyl group having 1 to 15 carbon atoms, or a substituted or unsubstituted phenyl group.

HAr may be represented by Formula 2 below, and Nap may be represented by Formula 3 or Formula 4 below.

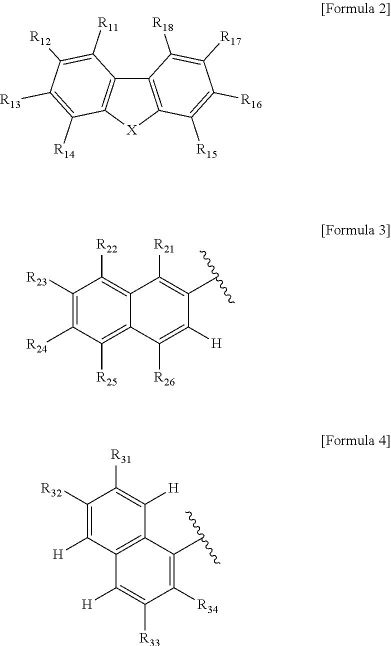

In Formula 2 above, X may be O or S. Any one of R₁₁ to R₁₄ may be a position linked to Formula 1 above, and the other positions of R₁₁ to R₁₄ excluding the linked position, and R₁₅ to R₁₈ may each independently be a hydrogen atom, a deuterium atom, a halogen atom, a substituted or unsubstituted alkyl group having 1 to 15 carbon atoms, or a substituted or unsubstituted phenyl group. However, when X is O and when linked to Formula 1 above at the R₁₁ position, the R₁₁ position is para to a nitrogen atom of Formula 1 above. When linked to Formula 1 above at the R₁₂ position, a case in which R₁₅ is a substituted or unsubstituted phenyl group is excluded. when linked to Formula 1 above at the R₁₄ position, Ar of Formula 1 above either includes a total of one aryl or heteroaryl group as a substituent or does not include any. When linked to Formula 1 above at the R₁₄ position and when L₁ is a direct linkage, a case in which R₁ is a phenyl group is excluded. In Formula 3 above, any one of R₂₁ to R₂₆ may be a substituted or unsubstituted phenyl group, and the others may each independently be a substituted or unsubstituted alkyl group having 1 to 15 carbon atoms, a halogen atom, a hydrogen atom, or a deuterium atom. However, when linked to Formula 1 above at the R₁₄ position of Formula 2 above and when L₁ is a direct linkage, cases in which R₂₁ or R₂₄ is a substituted or unsubstituted phenyl group may be excluded. In Formula 4 above, any one of R₃₁ to R₃₄ may be a substituted or unsubstituted phenyl group, and the others may each independently be a substituted or unsubstituted alkyl group having 1 to 15 carbon atoms, a halogen atom, a hydrogen atom, or a deuterium atom. However, when linked to Formula 1 at the R₁₄ position of Formula 2 above, a case in which R₃₄ is a substituted or unsubstituted phenyl group is excluded.

In an aspect, the hole transport region may include at least one of a hole injection layer, a hole transport layer, an electron blocking layer, or an auxiliary emission layer, and at least one of the hole injection layer, the hole transport layer, then electron blocking layer, or then auxiliary emission layer may include the amine compound.

In an aspect, the hole transport region may include a hole injection layer disposed on the first electrode, and a hole transport layer disposed on the hole injection layer, and the hole transport layer may include the amine compound.

In an aspect, Formula 1 above may be represented by Formula 1-1 or Formula 1-2 below.

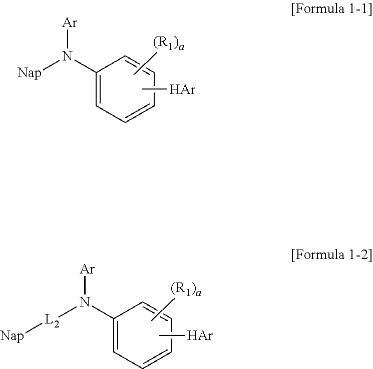

In Formula 1-2 above, L₂ may be a substituted or unsubstituted divalent phenyl, a substituted or unsubstituted divalent biphenyl, or a substituted or unsubstituted divalent dibenzofuran. In Formulas 1-1 and 1-2 above, Nap, Ar, HAr, R₁, and a are the same as defined in Formula 1 above.

In an aspect, Ar may be a substituted or unsubstituted phenyl group, or a substituted or unsubstituted naphthyl group, and
the substituted or unsubstituted substituent may be at least one of a hydrogen atom, a deuterium atom, a halogen atom, a phenyl group, a biphenyl group, or a naphthyl group.

In an aspect, Ar may be represented by any one of Ar-a to Ar-q, which will be described later.

In an aspect, the amine compound may be represented by any one of compounds from Compound Group 1, which will be described later.

In an aspect of the present disclosure, provided is an amine compound represented by Formula 1 above.

In an aspect of the present disclosure, an electronic device includes a display module including a plurality of light emitting elements,
wherein at least one of the plurality of light emitting elements includes a first electrode, a second electrode disposed on the first electrode, an emission layer disposed between the first electrode and the second electrode, and a hole transport region disposed between the first electrode and the emission layer and including an amine compound represented by Formula 1 above.

### BRIEF DESCRIPTION OF THE FIGURES

The accompanying drawings are included to provide a further understanding of the present disclosure, and are incorporated in and constitute a part of this specification. The drawings illustrate aspects of the present disclosure and, together with the description, serve to explain principles of the present disclosure. In the drawings:

FIG. 1 is a block diagram of an electronic device according to an aspect;

FIG. 2 shows schematic views of electronic devices according to an aspect;

FIG. 3 is a plan view of a display module according to an aspect;

FIG. 4 is a cross-sectional view showing a portion corresponding to line I-I′ of FIG. 3;

FIG. 5 is a cross-sectional view schematically showing a light emitting element according to an aspect;

FIG. 6 is a cross-sectional view schematically showing a light emitting element according to an aspect;

FIG. 7 is a cross-sectional view schematically showing a light emitting element according to an aspect;

FIG. 8 is a cross-sectional view schematically showing a light emitting element according to an aspect;

FIG. 9 is a cross-sectional view schematically showing a light emitting element according to an aspect;

FIG. 10 is a cross-sectional view showing a display module according to an aspect;

FIG. 11 is a cross-sectional view showing a display module according to an aspect;

FIG. 12 is a cross-sectional view showing a display module according to an aspect;

FIG. 13 is a cross-sectional view showing a display module according to an aspect;

FIG. 14 is a perspective view of an electronic device according to an aspect;

FIG. 15 is a perspective view of an electronic device according to an aspect; and

FIG. 16 is a view showing the inside of a vehicle in which an electronic device according to an aspect is disposed.

### DETAILED DESCRIPTION

The present disclosure may be modified in many alternate forms, and thus specific aspects will be exemplified in the drawings and described in detail. It should be understood, however, that it is not intended to limit the present disclosure to the particular forms disclosed, but rather, is intended to cover all modifications, equivalents, and alternatives falling within the spirit and scope of the present disclosure.

In this specification, it will be understood that when an element (or a region, a layer, a portion, or the like) is referred to as being “on”, “connected to” or “coupled to” another element, it may be directly disposed on, connected to, or coupled to the other element, or other elements may be disposed therebetween.

Like reference numerals or symbols refer to like elements throughout. In the drawings, the thickness, ratio, and size of the elements are exaggerated for effectively describing the technical contents. The term “and/or” includes any and all combinations of one or more of the associated listed elements.

It will be understood that, although the terms “first”, “second”, etc. may be used herein to describe various elements, the elements are not to be limited by these terms. These terms are only used to distinguish one element from another element. For instance, a first element could be termed a second element without departing from the scope of the present disclosure. Similarly, a second element could be termed a first element. The singular expressions “a”, “an” and “the” are intended to include the plural forms as well, unless the context clearly indicates otherwise.

In addition, the terms “below”, “under”, “on the lower side”, “above”, “over”, “on the upper side”, or the like may be used to describe the relationships between the elements illustrated in the drawings. These terms are relative concepts and are described on the basis of the directions indicated in the drawings.

It will be further understood that the terms “comprises, includes, has” and/or “comprising, including, having”, when used in this specification, specify the presence of stated features, numbers, steps, operations, elements, components or combinations thereof, but do not preclude the possibility of the presence or addition of one or more other features, numbers, steps, operations, elements, components, and/or combinations thereof.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Herein, the term “substituted or unsubstituted” may indicate that one is substituted or unsubstituted with at least one substituent selected from the group consisting of a deuterium atom, a halogen atom, a cyano group, a nitro group, an amine group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In addition, each of the substituents presented as an example above may be substituted or unsubstituted. For example, a biphenyl group may be interpreted as an aryl group or as a phenyl group substituted with a phenyl group.

Herein, the term “bonded to an adjacent group to form a ring” may indicate that one is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In addition, the rings formed by being bonded to each other may be linked to another ring to form a spiro structure.

Herein, the term “adjacent group” may indicate a substituent substituted for an atom which is directly linked to an atom substituted with a corresponding substituent, another substituent substituted for an atom which is substituted with a corresponding substituent, or a substituent sterically closest to a corresponding substituent. For example, two methyl groups in 1,2-dimethylbenzene may be interpreted as mutually “adjacent groups” and two ethyl groups in 1,1-diethylcyclopentane may be interpreted as mutually “adjacent groups”. In addition, two methyl groups in 4,5-dimethylphenanthrene may be interpreted as mutually “adjacent groups”.

Herein, examples of a halogen atom may include a fluorine atom, a chlorine atom, a bromine atom, or an iodine atom.

Herein, an alkyl group may be linear or branched. The number of carbon atoms in the alkyl group is 1 to 60, 1 to 50, 1 to 30, 1 to 20, 1 to 10, or 1 to 6. Examples of the alkyl group may include a methyl group, an ethyl group, an n-propyl group, an isopropyl group, an n-butyl group, a s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-icosyl group, a 2-ethylicosyl group, a 2-butylicosyl group, a 2-hexylicosyl group, a 2-octylicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, or the like, but are not limited thereto.

Herein, a cycloalkyl group may indicate a cyclic alkyl group. The number of carbon atoms in the cycloalkyl group is 3 to 60, 3 to 50, 3 to 30, 3 to 20, or 3 to 10. Examples of the cycloalkyl group include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, or the like, but are not limited thereto.

Herein, an alkenyl group indicates a hydrocarbon group including at least one carbon double bond in the middle or end of an alkyl group having 2 or more carbon atoms. The alkenyl group may be linear or branched. The number of carbon atoms is not particularly limited, but is 2 to 60, 2 to 30, 2 to 20, or 2 to 10. Examples of the alkenyl group include a vinyl group, a 1-butenyl group, a 1-pentenyl group, a 1,3-butadienyl aryl group, a styrenyl group, a styryl vinyl group, or the like, but are not limited thereto.

Herein, an alkynyl group indicates a hydrocarbon group including at least one carbon triple bond in the middle or end of an alkyl group having 2 or more carbon atoms. The alkynyl group may be linear or branched. The number of carbon atoms is not particularly limited, but is 2 to 30, 2 to 20, or 2 to 10. Specific examples of the alkynyl group may include an ethynyl group, a propynyl group, or the like, but are not limited thereto.

Herein, a hydrocarbon ring group indicates any functional group or substituent derived from an aliphatic hydrocarbon ring. The hydrocarbon ring group may be a saturated hydrocarbon ring group having 6 to 60, 6 to 30, 5 to 30, or 5 to 20 ring-forming carbon atoms.

Herein, an aryl group indicates any functional group or substituent derived from an aromatic hydrocarbon ring. The aryl group may be a monocyclic aryl group or a polycyclic aryl group. The number of ring-forming carbon atoms in the aryl group may be 6 to 60, 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, or the like, but are not limited thereto.

Herein, a fluorenyl group may be substituted, and two substituents may be bonded to each other to form a spiro structure. An example that the fluorenyl group is substituted is as follows. However, aspects of the present disclosure are not limited thereto.

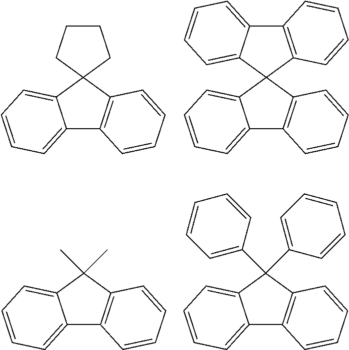

Herein, a heterocyclic group indicates any functional group or substituent derived from a ring containing at least one of B, O, N, P, Si, or S as a hetero atom. The heterocyclic group includes an aliphatic heterocyclic group and an aromatic heterocyclic group. The aromatic heterocyclic group may be a heteroaryl group. The aliphatic heterocyclic group and the aromatic heterocyclic group may be monocyclic or polycyclic.

Herein, the heterocyclic group may contain at least one of B, O, N, P, Si, or S as a hetero atom. When the heterocyclic group contains two or more hetero atoms, the two or more hetero atoms may be the same as or different from each other. The heterocyclic group may be a monocyclic heterocyclic group or a polycyclic heterocyclic group, and include a heteroaryl group. The number of ring-forming carbon atoms in the heterocyclic group may be 2 to 60, 2 to 30, 5 to 30, 2 to 20, or 2 to 10.

Herein, the aliphatic heterocyclic group may contain at least one of B, O, N, P, Si, or S as a hetero atom. The number of ring-forming carbon atoms in the aliphatic heterocyclic group may be 2 to 60, 2 to 30, 2 to 20, or 2 to 10. Examples of the aliphatic heterocyclic group include an oxirane group, a thiirane group, a pyrrolidine group, a piperidine group, a tetrahydrofuran group, a tetrahydrothiophene group, a thiane group, a tetrahydropyran group, a 1,4-dioxane group, or the like, but are not limited to thereto.

Herein, a heteroaryl group may contain at least one of B, O, N, P, Si, or S as a hetero atom. When the heteroaryl group contains two or more hetero atoms, the two or more hetero atoms may be the same as or different from each other. The heteroaryl group may be a monocyclic heteroaryl group or a polycyclic heteroaryl group. The number of ring-forming carbon atoms in the heteroaryl group may be 2 to 60, 2 to 30, 2 to 20, or 2 to 10. Examples of the heteroaryl group include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzoimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, or the like, but are not limited thereto.

Herein, the above description of the aryl group may also apply to an arylene group, except that the arylene group is a divalent group. The above description of the heteroaryl group may also apply to a heteroarylene group, except that the heteroarylene group is a divalent group.

Herein, a silyl group includes an alkyl silyl group and an aryl silyl group. Examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, or the like, but are not limited thereto.

Herein, the number of carbon atoms in a carbonyl group is not particularly limited, but may be 1 to 40, 1 to 30, or 1 to 20. For example, the carbonyl group may have the following structure, but is not limited thereto.

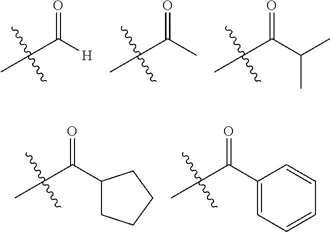

Herein, the number of carbon atoms in a sulfinyl group and a sulfonyl group is not particularly limited, but may be 1 to 30. The sulfinyl group may include an alkyl sulfinyl group or an aryl sulfinyl group. The sulfonyl group may include an alkyl sulfonyl group or an aryl sulfonyl group.

Herein, a thio group may include an alkyl thio group and an aryl thio group. The thio group may indicate the one that a sulfur atom is bonded to the alkyl group or the aryl group as defined above. Examples of the thio group include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, or the like, but are not limited to thereto.

Herein, an oxy group may indicate the one that an oxygen atom is bonded to the alkyl group or the aryl group as defined above. The oxy group may include an alkoxy group or an aryl oxy group. The alkoxy group may be linear, branched, or cyclic. The number of carbon atoms in the alkoxy group is not particularly limited, but may be, for example, 1 to 20, or 1 to 10. Examples of the oxy group include methoxy, ethoxy, n-propoxy, isopropoxy, butoxy, pentyloxy, hexyloxy, octyloxy, nonyloxy, decyloxy, benzyloxy, or the like, but are not limited thereto.

Herein, a boron group may indicate the one that a boron atom is bonded to the alkyl group or the aryl group as defined above. The boron group includes an alkyl boron group or an aryl boron group. Examples of the boron group include a dimethylboron group, a diethylboron group, a t-butylmethylboron group, a diphenylboron group, a phenylboron group, or the like, but are not limited thereto.

Herein, the number of carbon atoms in an amine group is not particularly limited, but may be 1 to 50, 1 to 30, or 1 to 20. The amine group may include an alkyl amine group or an aryl amine group. Examples of the amine group include a methylamine group, a dimethylamine group, a phenylamine group, a diphenylamine group, a naphthylamine group, a 9-methyl-anthracenylamine group, or the like, but are not limited thereto.

Herein, the above-described examples of the alkyl group also apply to an alkyl group from an alkylthio group, an alkyl sulfoxy group, an alkylaryl group, an alkylamino group, an alkyl boron group, an alkyl silyl group, or an alkyl amine group.

Herein, the above-described examples of the aryl group also apply to an aryl group from an aryloxy group, an arylthio group, an aryl sulfoxy group, an arylamino group, an aryl boron group, an aryl silyl group, or an aryl amine group.

Herein, a direct linkage may indicate a single bond. Herein,

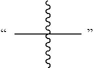

and “ [symbol] ” indicate positions to be linked.

Hereinafter, aspects of the present disclosure will be described with reference to the accompanying drawings.

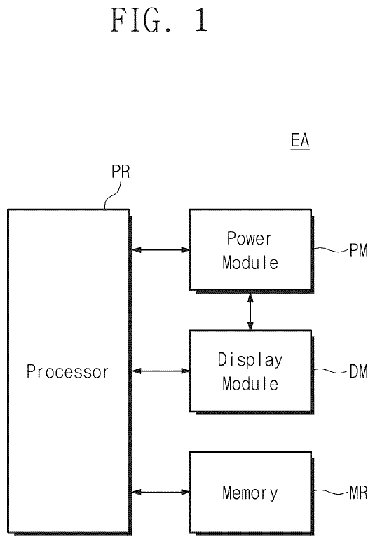

FIG. 1 is a block diagram of an electronic device according to an aspect. Referring to FIG. 1, an electronic device EA according to an aspect may include a display module DM, a processor PR, a memory MR, and a power module PM.

The processor PR may include at least one of a central processing unit (CPU), an application processor (AP), a graphic processing unit (GPU), a communication processor (CP), an image signal processor (ISP), or a controller.

The memory MR may store data information required for the operation of the processor PR or the display module DM. When the processor PR executes an application stored in the memory MR, image data signals and/or input control signals are transmitted to the display module DM, and the display module DM may process the received signal and output image information through a display screen. The display module DM may include a display panel that displays an image.

The power module PM may include a power supply module such as a power adapter or a battery device, and a power conversion module that converts power supplied by the power supply module to generate power required for the operation of the electronic device EA.

At least one of the components of the electronic device EA described above may be included in a display module according to aspect, which will be described later, and a display device according to an aspect including the same. In addition, some of the individual modules functionally included in one module may be included in the display device, and others may be separately provided from the display device. For example, the display device may include a display module DM, and the processor PR, the memory MR, and the power module PM may be provided in the form of other devices within the electronic device EA, rather than the display device.

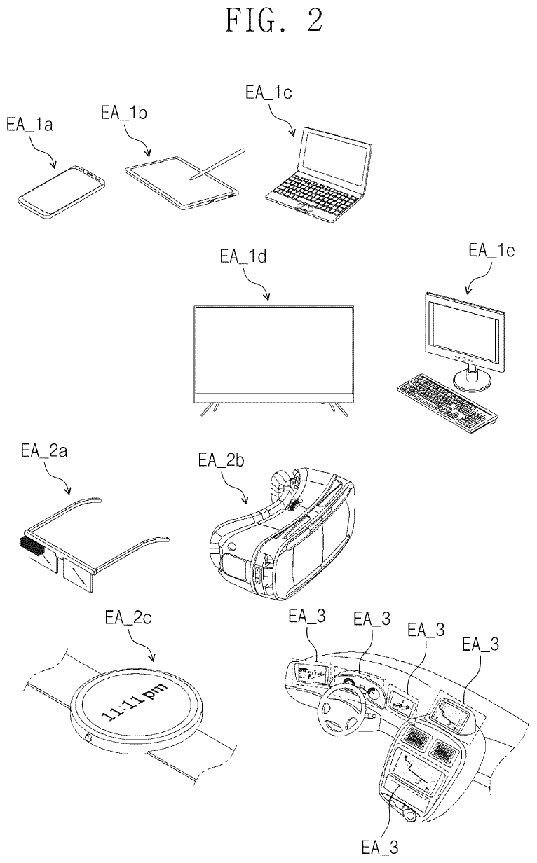

FIG. 2 shows schematic views of aspects of various electronic devices.

Referring to FIG. 2, various electronic devices including the display module according to an aspect may include electronic devices for displaying images, such as a smart phone EA_1a, a tablet PC EA_1b, a laptop EA_1c, a TV EA_1d, or a desk monitor EA_1e, wearable electronic devices such as smart glasses EA_2a, a head mounted display EA_2b, or a smart watch EA_2c, or vehicle electronic devices EA_3 such as a center information display (CID) or a room mirror display disposed on an instrument panel, a center fascia, or a dashboard of a vehicle.

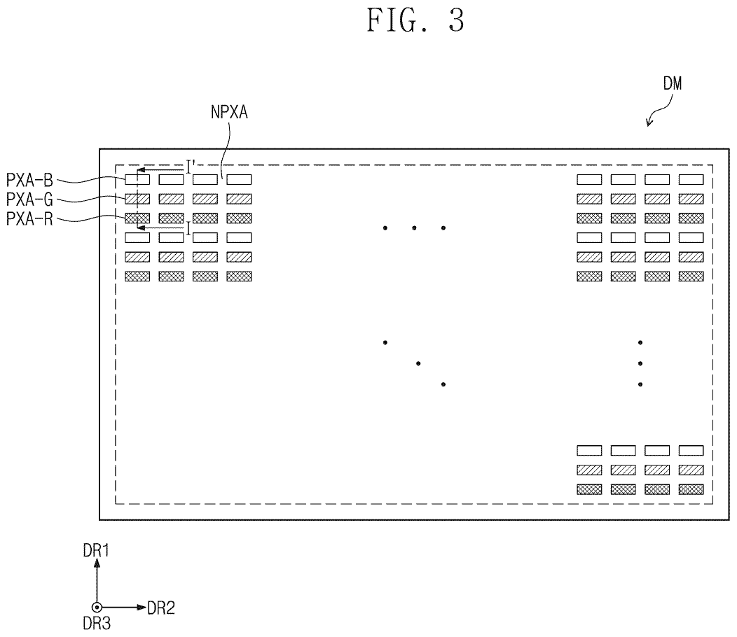

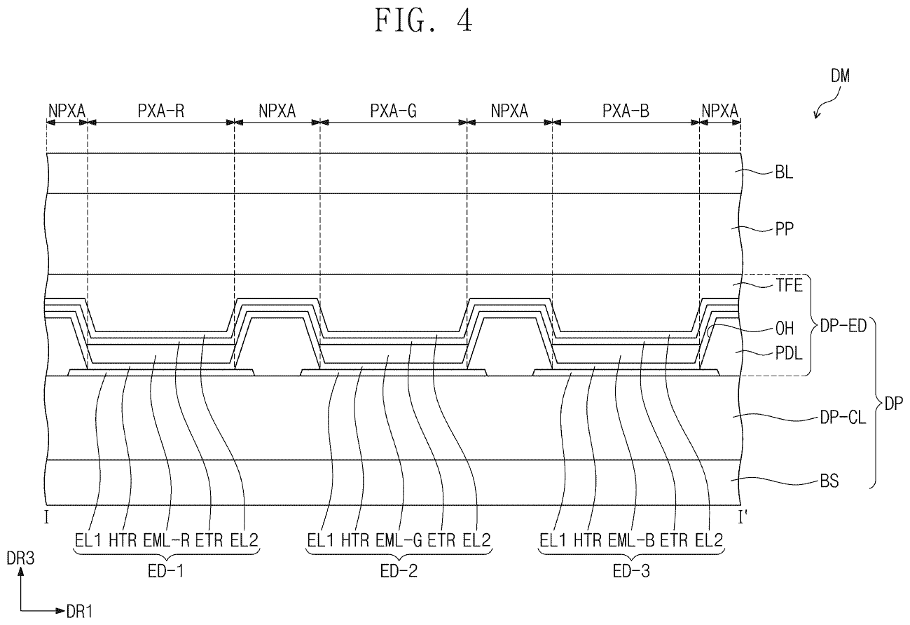

FIG. 3 is a plan view showing an aspect of a display module DM. FIG. 4 is a cross-sectional view showing a display module DM according to an aspect. FIG. 4 is a cross-sectional view showing a portion corresponding to line I-I′ of FIG. 3.

The display module DM may include a plurality of light emitting elements ED-1, ED-2, and ED-3. In an aspect, the display module DM may include a display panel DP including a plurality of light emitting elements ED-1, ED-2, and ED-3 and further include an optical layer PP disposed on the display panel DP.

The display panel DP may include a base layer BS, a circuit layer DP-CL provided on the base layer BS, and a display element layer DP-ED. The display element layer DP-ED may include pixel defining films PDL, a plurality of light emitting elements ED-1, ED-2, and ED-3 disposed between the pixel defining films PDL, and an encapsulation layer TFE disposed on the plurality of light emitting elements ED-1, ED-2, and ED-3.

The base layer BS may be a member providing a base surface on which the display element layer DP-ED is disposed. The base layer BS may be a glass substrate, a metal substrate, a plastic substrate, or the like. However, aspects of the present disclosure are not limited thereto, and the base layer BS may be an inorganic layer, an organic layer, or a composite material layer.

In an aspect, the circuit layer DP-CL may be disposed on the base layer BS, and the circuit layer DP-CL may include a plurality of transistors (not shown). The transistors (not shown) may each include a control electrode, an input electrode, and an output electrode. For example, the circuit layer DP-CL may include a switching transistor and a driving transistor for driving the light emitting elements ED-1, ED-2 and ED-3 of the display element layer DP-ED.

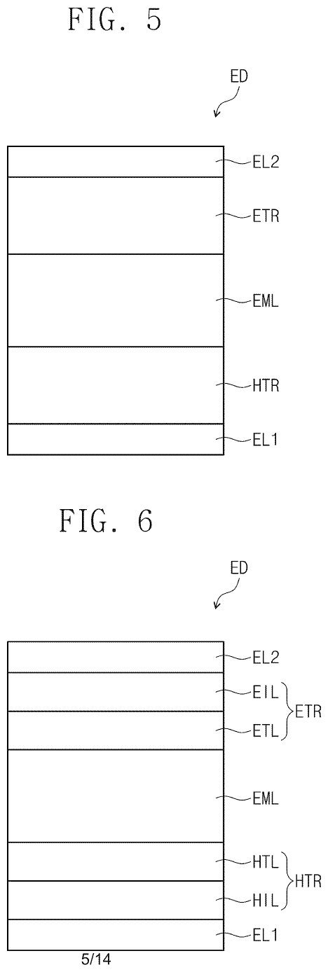

The light emitting elements ED-1, ED-2, and ED-3 may each have a structure of a light emitting element ED according to an aspect from FIGS. 5 to 9, which will be described later. The light emitting elements ED-1, ED-2, and ED-3 may each include a first electrode EL1, a hole transport region HTR, emission layers EML-R, EML-G, and EML-B, an electron transport region ETR, and a second electrode EL2.

The optical layer PP may be disposed on the display panel DP to control reflected light in the display panel DP due to external light. The optical layer PP may include, for example, a polarizing layer or a color filter layer. Unlike what is shown in the drawings, the optical layer PP may not be provided in the display module DM according to an aspect.

A base substrate BL may be disposed on the optical layer PP. The base substrate BL may be a member providing a base surface on which the optical layer PP is disposed. The base substrate BL may be a glass substrate, a metal substrate, a plastic substrate, or the like. However, aspects of the present disclosure are not limited thereto, and the base substrate BL may be an inorganic layer, an organic layer, or a composite material layer. In addition, unlike what is shown, the base substrate BL may not be provided in an aspect.

The display module DM according to an aspect may further include a filling layer (not shown). The filling layer (not shown) may be disposed between a display element layer DP-ED and the base substrate BL. The filling layer (not shown) may be an organic material layer. The filling layer (not shown) may include at least one of an acrylic resin, a silicone-based resin, or an epoxy-based resin.

FIG. 4 shows an aspect in which the emission layers EML-R, EML-G, and EML-B of the light emitting elements ED-1, ED-2, and ED-3 are disposed in openings OH defined in the pixel defining films PDL, and the hole transport region HTR, the electron transport region ETR, and the second electrode EL2 are provided as a common layer throughout the light emitting elements ED-1, ED-2, and ED-3. However, aspects of the present disclosure are not limited thereto, and unlike what is shown in FIG. 4, in an aspect, the hole transport region HTR and the electron transport region ETR may be provided to be patterned inside the openings OH defined in the pixel defining films PDL. For example, in an aspect, the hole transport region HTR, the emission layers EML-R, EML-G, and EML-B, and the electron transport region ETR, or the like of the light emitting elements ED-1, ED-2, and ED-3 may be patterned and provided through an inkjet printing method.

The encapsulation layer TFE may cover the light emitting elements ED-1, ED-2 and ED-3. The encapsulation layer TFE may seal the display element layer DP-ED. The encapsulation layer TFE may be a thin film encapsulation layer. The encapsulation layer TFE may be a single layer or a stack layer of a plurality of layers. The encapsulation layer TFE includes at least one insulating layer. The encapsulation layer TFE according to an aspect may include at least one inorganic film (hereinafter, an encapsulation inorganic film). In addition, the encapsulation layer TFE according to an aspect may include at least one organic film (hereinafter, an encapsulation organic film) and at least one encapsulation inorganic film.

The encapsulation inorganic film protects the display element layer DP-ED from moisture/oxygen, and the encapsulation organic film protects the display element layer DP-ED from foreign substances such as dust particles. The encapsulation inorganic film may include silicon nitride, silicon oxy nitride, silicon oxide, titanium oxide, aluminum oxide, or the like, but is not particularly limited thereto. The encapsulation organic film may include an acrylic compound, an epoxy-based compound, or the like. The encapsulation organic film may include a photopolymerizable organic material, and is not particularly limited.

The encapsulation layer TFE may be disposed on the second electrode EL2, and may be disposed to fill the openings OH.

Referring to FIGS. 3 and 4, the display module DM may include a non-light emitting region NPXA and light emitting regions PXA-R, PXA-G, and PXA-B. The light emitting regions PXA-R, PXA-G, and PXA-B may each be a region emitting light generated from each of the light emitting elements ED-1, ED-2, and ED-3. The display module DM may include a first light emitting region PXA-R, a second light emitting region PXA-G, and a third light emitting region PXA-B.

The first to third light emitting regions PXA-R, PXA-G, and PXA-B may each be a region separated by the pixel defining film PDL. The non-light emitting region NPXA may be a region between neighboring light emitting regions PXA-R, PXA-G, and PXA-B, and may correspond to the pixel defining films PDL. Herein, the light emitting regions PXA-R, PXA-G, and PXA-B may each correspond to a pixel. The pixel defining films PDL may separate the light emitting elements ED-1, ED-2 and ED-3. The emission layers EML-R, EML-G, and EML-B of the light emitting elements ED-1, ED-2 and ED-3 may be disposed in the openings OH defined by the pixel defining films PDL and thus be separated.

The light emitting regions PXA-R, PXA-G, and PXA-B may be divided into a plurality of groups according to the color of light generated from the light emitting elements ED-1, ED-2, and ED-3. In the display module DM according to an aspect shown in FIGS. 3 and 4, three light emitting regions PXA-R, PXA-G, and PXA-B which emit red light, green light, and blue light, are shown as an example. For example, the first light emitting region PXA-R may be referred to as a red light emitting region, the second light emitting region PXA-G may be referred to as a green light emitting region, and the third light emitting region PXA-B may be referred to as a blue light emitting region.

In the display module DM according to an aspect, the plurality of light emitting elements ED-1, ED-2, and ED-3 may emit light having different wavelength ranges. For example, in an aspect, the display module DM may include a first light emitting element ED-1 emitting red light, a second light emitting element ED-2 emitting green light, and a third light emitting element ED-3 emitting blue light. That is, the red light emitting region PXA-R, the green light emitting region PXA-G, and the blue light emitting region PXA-B of the display module DM may correspond to the first light emitting element ED-1, the second light emitting element ED-2, and the third light emitting element ED-3, respectively.

However, aspects of the present disclosure are not limited thereto, and the first to third light emitting elements ED-1, ED-2 and ED-3 may emit light in the same wavelength range or emit light in at least one different wavelength range. For example, the first to third light emitting elements ED-1, ED-2, and ED-3 may all emit blue light.

The light emitting regions PXA-R, PXA-G, and PXA-B in the display module DM according to an aspect may be arranged in the form of a stripe. Referring to FIG. 1, a plurality of first light emitting regions PXA-R, a plurality of second light emitting regions PXA-G, and a plurality of third light emitting regions PXA-B may each be arranged along the second directional axis DR2. In addition, the first light emitting region PXA-R, the second light emitting region PXA-G, and the third light emitting region PXA-B may be alternately arranged in that order along the first directional axis DR1.

FIGS. 3 and 4 show that the light emitting regions PXA-R, PXA-G, and PXA-B are all similar in size, but aspects of the present disclosure are not limited thereto, and the light emitting regions PXA-R, PXA-G, and PXA-B may be different in size from each other according to wavelength range of emitted light. The areas of the light emitting regions PXA-R, PXA-G, and PXA-B may indicate areas when viewed on a plane defined by the first directional axis DR1 and the second directional axis DR2.

The arrangement of the light emitting regions PXA-R, PXA-G, and PXA-B is not limited to what is shown in FIG. 1, and the order in which the first light emitting region PXA-R, the second light emitting region PXA-G, and the third light emitting region PXA-B are arranged comes with varied combination according to display quality characteristics required for the display module DM and a display device DD including the same. For example, the light emitting regions PXA-R, PXA-G, and PXA-B may be arranged in the form of a pentile (PENTILE™) or a diamond (Diamond Pixel™).

In addition, areas of each of the light emitting regions PXA-R, PXA-G, and PXA-B may be different in size from one another. For example, in an aspect, the second pixel region PXA-G corresponding to a green light emitting region may be smaller than the third pixel region PXA-B corresponding to a blue light emitting region in size, but aspects of the present disclosure are not limited thereto.

Hereinafter, FIGS. 5 to 9 are cross-sectional views schematically showing a light emitting element according to an aspect. The light emitting element ED according to an aspect may include a first electrode EL1, a hole transport region HTR, an emission layer EML, an electron transport region ETR, and a second electrode EL2, which are sequentially stacked.

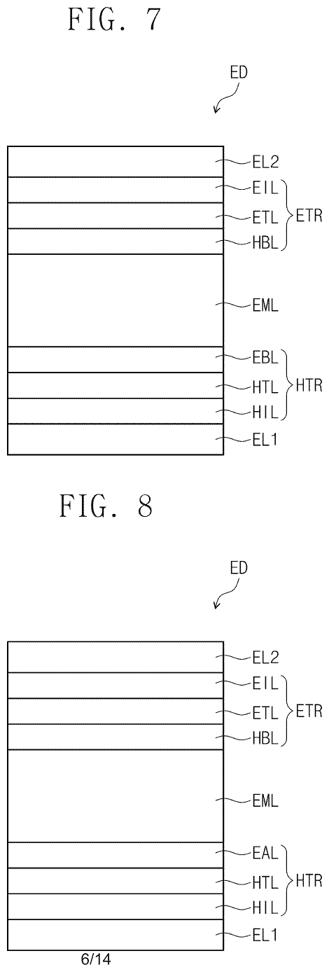

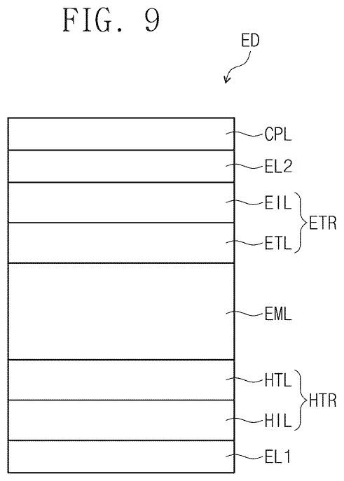

FIG. 6 shows, compared with FIG. 5, a cross-sectional view of a light emitting element ED according to an aspect in which the hole transport region HTR includes a hole injection layer HIL and a hole transport layer HTL, and the electron transport region ETR includes an electron injection layer EIL and an electron transport layer ETL. In addition, FIG. 7 shows, compared with FIG. 5, a cross-sectional view of a light emitting element ED according to an aspect, in which the hole transport region HTR includes a hole injection layer HIL, a hole transport layer HTL, and an electron blocking layer EBL, and the electron transport region ETR includes an electron injection layer EIL, an electron transport layer ETL, and a hole blocking layer HBL. FIG. 8 shows, compared with FIG. 5, a cross-sectional view of a light emitting element ED according to an aspect in which the hole transport region HTR includes a hole injection layer HIL, a hole transport layer HTL, and an auxiliary emission layer EAL, and the electron transport region ETR includes an electron injection layer EIL, an electron transport layer ETL, and a hole blocking layer HBL. FIG. 9 shows, compared with FIG. 6, a cross-sectional view of a light emitting element ED according to an aspect, in which a capping layer CPL disposed on the second electrode EL2 is provided.

The first electrode EL1 has conductivity. The first electrode EL1 may be formed of a metal material, a metal alloy, or a conductive compound. The first electrode EL1 may be an anode or a cathode. However, aspects of the present disclosure are not limited thereto. In addition, the first electrode EL1 may be a pixel electrode. The first electrode EL1 may be a transmissive electrode, a transflective electrode, or a reflective electrode. The first electrode EL1 may be a transmissive electrode, a transflective electrode, or a reflective electrode. The first electrode EL1 may include at least one selected from Ag, Mg, Cu, Al, Pt, Pd, Au, Ni, Nd, Ir, Cr, Li, Ca, LiF, Mo, Ti, W, In, Sn, or Zn, at least two compounds selected therefrom, two or more mixtures selected therefrom, or an oxide thereof.

When the first electrode EL1 is a transmissive electrode, the first electrode EL1 may include a transparent metal oxide such as indium tin oxide (ITO), indium zinc oxide (IZO), zinc oxide (ZnO), or indium tin zinc oxide (ITZO). When the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca (a stack structure of LiF and Ca), LiF/Al (a stack structure of LiF and Al), Mo, Ti, W, or a compound thereof or a mixture thereof (e.g., a mixture of Ag and Mg). Alternatively, the first electrode EL1 may have a multilayer structure including a reflective film or a transflective film formed of the above-described materials, and a transparent conductive film formed of indium tin oxide (ITO), indium zinc oxide (IZO), zinc oxide (ZnO), indium tin zinc oxide (ITZO), or the like. For example, the first electrode EL1 may have a three-layer structure of ITO/Ag/ITO, but is not limited thereto. In addition, aspects of the present disclosure are not limited thereto, and the first electrode EL1 may include the above-described metal materials, a combination of two or more metal materials selected from the above-described metal materials, or oxides of the above-described metal materials. The first electrode EL1 may have a thickness of about 700 Å to about 10000 Å. For example, the first electrode EL1 may have a thickness of about 1000 Å to about 3000 Å.

The hole transport region HTR is provided on the first electrode EL1. The hole transport region HTR may include at least one of a hole injection layer HIL, a hole transport layer HTL, an auxiliary emission layer EAL, or an electron blocking layer EBL. The hole transport region HTR may have, for example, a thickness of about 50 Å to about 15000 Å. The auxiliary emission layer EAL may also be referred to as a buffer layer.

The hole transport region HTR may have a single layer formed of a single material, a single layer formed of a plurality of different materials, or a multilayer structure having a plurality of layers formed of a plurality of different materials.

For example, the hole transport region HTR may have a single-layer structure formed of the hole injection layer HIL or the hole transport layer HTL, or a single-layer structure formed of a hole injection material or a hole transport material. For example, the hole transport region HTR may have a single-layer structure formed of a plurality of different materials, or a structure of hole injection layer HIL/hole transport layer HTL, hole injection layer HIL/hole transport layer HTL/auxiliary emission layer EAL, hole injection layer HIL/auxiliary emission layer EAL, hole transport layer HTL/auxiliary emission layer EAL, hole injection layer HIL/hole transport layer HTL/auxiliary emission layer EAL, or hole injection layer HIL/hole transport layer HTL/electron blocking layer EBL stacked in order from the first electrode EL1, but aspects of the present disclosure are not limited thereto.

The hole transport region HTR may be formed using various methods such as a vacuum deposition method, a spin coating method, a cast method, a Langmuir-Blodgett (LB) method, an inkjet printing method, a laser printing method, or a laser induced thermal imaging (LITI) method.

The light emitting element ED according to an aspect may include the amine compound according to an aspect represented by Formula 1 below in the hole transport region HTR. At least one of the hole injection layer HIL, the hole transport layer HTL, the electron blocking layer EBL, or the auxiliary emission layer EAL may include the amine compound according to an aspect. For example, the light emitting element ED according to an aspect may include the amine compound according to an aspect in the hole transport layer HTL. The light emitting element ED according to an aspect may include the amine compound according to an aspect represented by Formula 1 below in the hole transport region HTR. In an aspect, at least one of the hole injection layer HIL, the hole transport layer HTL, the electron blocking layer EBL, or the auxiliary emission layer EAL may include the amine compound according to an aspect represented by Formula 1. For example, the light emitting element ED according to an aspect may include the amine compound according to an aspect in the hole transport layer HTL.

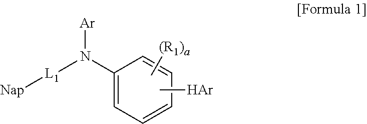

The amine compound according to an aspect may include first to third substituents directly or indirectly bonded to a nitrogen atom (N). The first substituent may be a substituted or unsubstituted phenylnaphthyl group (Nap). The second substituent may be a phenyl derivative substituted with a dibenzoheterol group (HAr). The third substituent may be an aryl derivative (Ar), and the aryl derivative may be a substituted or unsubstituted phenyl group or a substituted or unsubstituted naphthyl group. The first substituent may be bonded directly to the nitrogen atom (N) or may be bonded to the nitrogen atom (N) via a linker. The second substituent and the third substituent may each be directly bonded to the nitrogen atom (N). Meanwhile, the amine compound according to an aspect may be a monoamine compound that does not include an additional amine substituent.

In the amine compound according to an aspect, the substituted or unsubstituted phenylnaphthyl group (Nap) may be bonded directly to the nitrogen atom at a C1 position or a C2 position, or may be bonded to the nitrogen atom via a linker, as shown below. Examples of the phenylnaphthyl group listed below only show a core structure of the phenylnaphthyl group and bonding positions thereof, and other substituents are not provided.

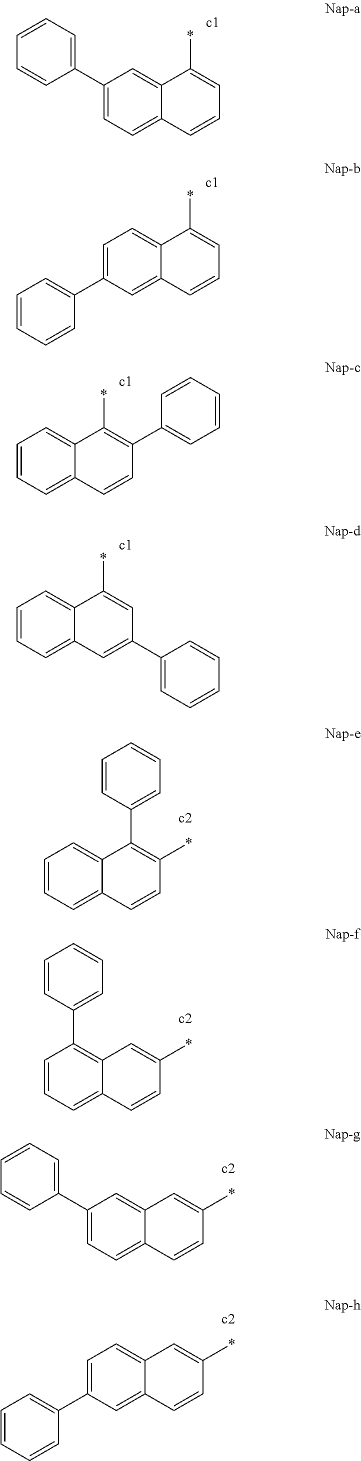

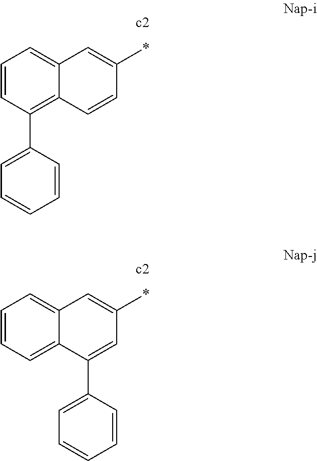

In addition, in the amine compound according to an aspect, the phenyl derivative substituted with a dibenzoheterol group (HAr) may be bonded directly to the nitrogen atom at a C3 position, as shown below. The dibenzoheteroyl group may be linked to the phenyl group so as to be positioned ortho, meta, or para to the nitrogen atom. Examples of the phenyl derivative substituted with a dibenzoheteroyl group listed below only show core structures of the phenyl group and the dibenzoheteroyl group substituted thereto, and bonding positions to the nitrogen atom, and other substituents are not provided.

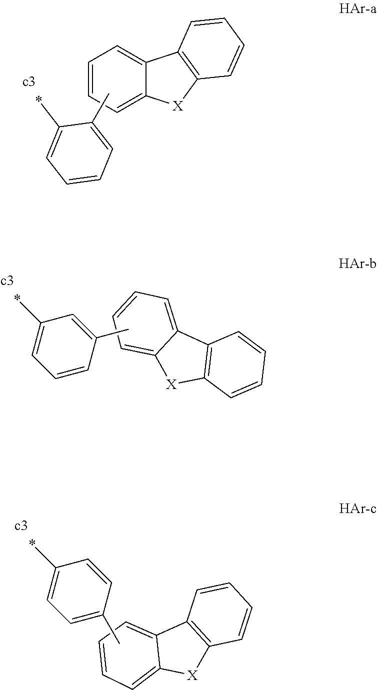

In addition, in the amine compound according to an aspect, the aryl derivative (Ar) may be directly bonded to the nitrogen atom at a C4 position, a C5 position, or a C6 position, as shown below. The aryl derivative may be a substituted or unsubstituted phenyl group, or a substituted or unsubstituted naphthyl group. Examples of the aryl derivative listed below only show a core structure of the aryl group and bonding positions to the nitrogen atom, and other substituents are not provided.

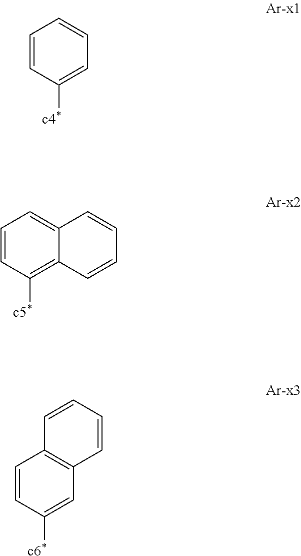

The amine compound according to an aspect includes the first to third substituents, which control charge balance through a steric effect, thereby exhibiting excellent charge transport properties. That is, the amine compound according to an aspect includes a phenylnaphthyl group bonded to the nitrogen atom at a specific position, a phenyl derivative substituted with a dibenzoheteroyl group, and an aryl derivative, thereby exhibiting excellent charge transport properties and material stability, contributing to high efficiency and long lifespan of light emitting elements.

In Formula 1, Ar may be a substituted or unsubstituted phenyl group without including an aryl group or a heteroaryl group having 16 or more ring-forming carbon atoms as a substituent, or a substituted or unsubstituted naphthyl group,

In an aspect, Ar may be represented by any one of Formulas Ar-1 to Ar-3 below.

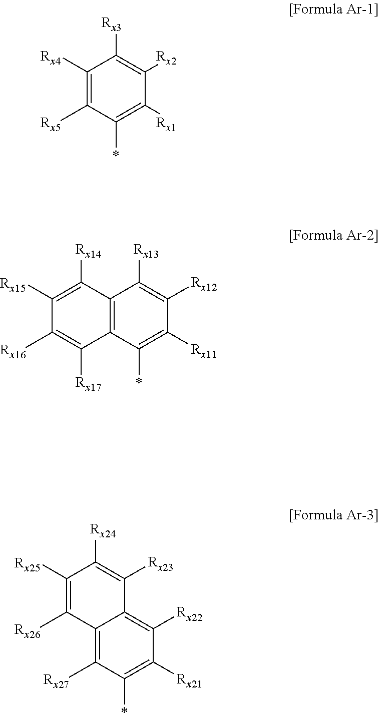

In Formulas Ar-1 to Ar-3 above, Rₓ₁ to Rₓ₅, Rₓ₁₁ to Rₓ₁₆, and Rₓ₂₁ to Rₓ₂₇ may each independently be a hydrogen atom, a deuterium atom, a halogen atom, a substituted or unsubstituted alkyl group having 1 to 15 carbon atoms, a substituted or unsubstituted aryl group having 6 to 15 ring-forming carbon atoms, or a substituted or unsubstituted heteroaryl group having 5 to 15 ring-forming carbon atoms.

However, when dibenzoheteroyl is linked to a phenyl group directly bonded to the nitrogen atom at the R₁₄ position, a case in which Ar includes a total of two or more aryl and heteroaryl groups as a substituent is excluded. That is, when dibenzoheteroyl is linked to a phenyl group directly bonded to the nitrogen atom at the R₁₄ position, only any one of Rₓ₁ to Rₓ₅ is a substituted or unsubstituted aryl group having 6 to 15 ring-forming carbon atoms, or a substituted or unsubstituted heteroaryl group having 5 to 15 ring-forming carbon atoms, and the others are each independently a hydrogen atom, a deuterium atom, a halogen atom, or a substituted or unsubstituted alkyl group having 1 to 15 carbon atoms. In addition, when dibenzoheteroyl is linked to a phenyl group directly bonded to the nitrogen atom at the R₁₄ position, only any one of Rₓ₁₁ to Rₓ₁₆ is a substituted or unsubstituted aryl group having 6 to 15 ring-forming carbon atoms, or a substituted or unsubstituted heteroaryl group having 5 to 15 ring-forming carbon atoms, and the others are each independently a hydrogen atom, a deuterium atom, a halogen atom, or a substituted or unsubstituted alkyl group having 1 to 15 carbon atoms. In addition, when dibenzoheteroyl is linked to a phenyl group directly bonded to the nitrogen atom at the R₁₄ position, only any one of Rₓ₂₁ to Rₓ₂₇ is a substituted or unsubstituted aryl group having 6 to 15 ring-forming carbon atoms, or a substituted or unsubstituted heteroaryl group having 5 to 15 ring-forming carbon atoms, and the others are each independently a hydrogen atom, a deuterium atom, a halogen atom, or a substituted or unsubstituted alkyl group having 1 to 15 carbon atoms.

In an aspect, Ar may be a substituted or unsubstituted phenyl group, or a substituted or unsubstituted naphthyl group, and the substituent in the substituted or unsubstituted group may be a hydrogen atom, a deuterium atom, a halogen atom, a phenyl group, a biphenyl group, or a naphthyl group. For example, Ar may be represented by any one of Ar-a to Ar-q below. However, aspects of the present disclosure are not limited thereto.

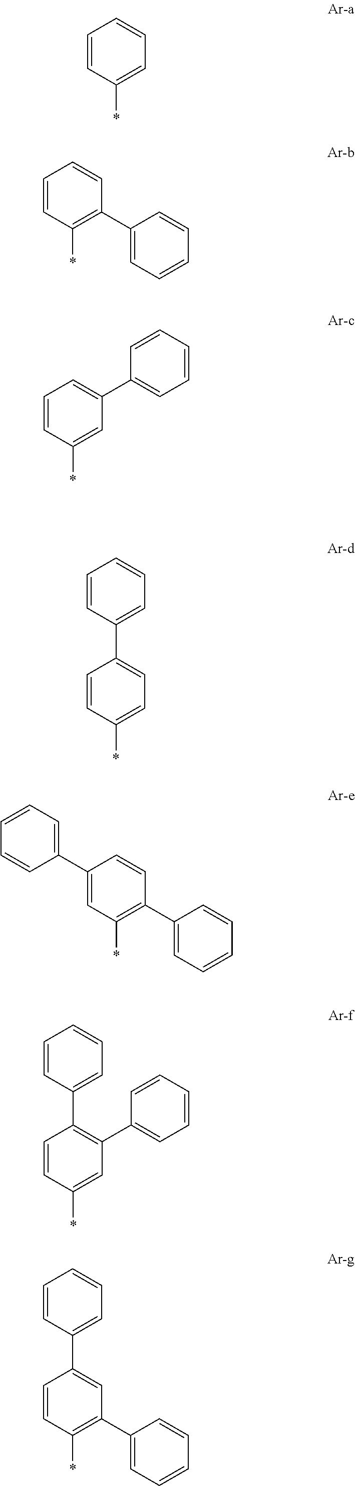

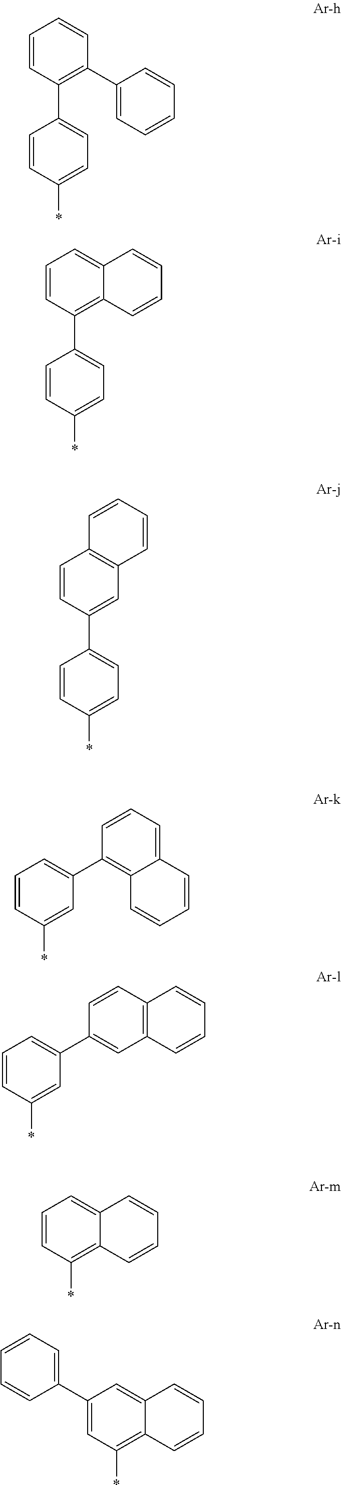

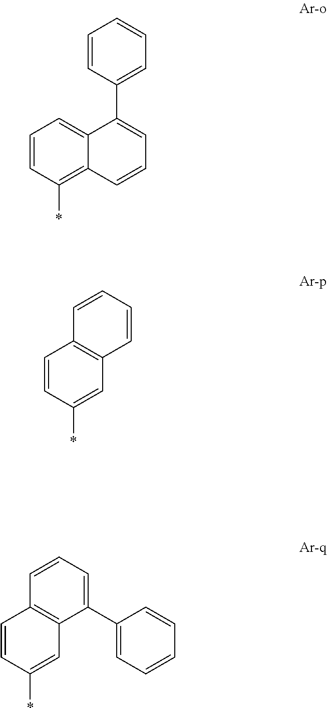 [SMILES: *c1cccc2c(-c3ccccc3)cccc12 ; *c1ccc2ccccc2c1 ; *c1ccc2cccc(-c3ccccc3)c2c1]

In Formula 1, a may be an integer of 0 to 4.

In Formula 1, R₁ may be a hydrogen atom, a deuterium atom, a halogen atom, a substituted or unsubstituted alkyl group having 1 to 15 carbon atoms, or a substituted or unsubstituted phenyl group.

When a is an integer of 2 or greater, a plurality of R₁'s may all be the same or at least one may be different from the others.

In Formula 1, HAr may be a dibenzoheterol group. The dibenzoheterol group may be linked to a phenyl group so as to be positioned ortho, meta, or para to the nitrogen atom of the amine compound.

In an aspect, HAr may be represented by Formula 2 below.

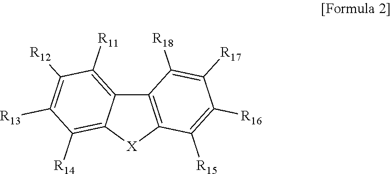

In Formula 2, X may be O or S. That is, the amine compound according to an aspect may include a phenyl derivative substituted with a dibenzofuran group or a phenyl derivative substituted with a dibenzothiophene group, as the second substituent.

In Formula 2, any one of R₁₁ to R₁₄ may be a position linked to Formula 1, and the others may each independently be a substituted or unsubstituted alkyl group having 1 to 15 carbon atoms, a halogen atom, a hydrogen atom, or a deuterium atom. R₁₅ to R₁₈ may each independently be a hydrogen atom, a deuterium atom, a halogen atom, a substituted or unsubstituted alkyl group having 1 to 15 carbon atoms, or a substituted or unsubstituted phenyl group.

However, when X is O and when linked to a phenyl group at the R₁₁ position, the R₁₁ position is para to the nitrogen atom of the amine compound. That is, in the amine compound according to an aspect, a case in which R₁₁ is positioned meta to the nitrogen atom, as in Formula HAr-1 below, and a case in which R₁₁ is positioned ortho to the nitrogen atom, as in HAr-2, are excluded. In Formulas HAr-1 and HAr-2 below, C3 is a position directly bonded to the nitrogen atom.

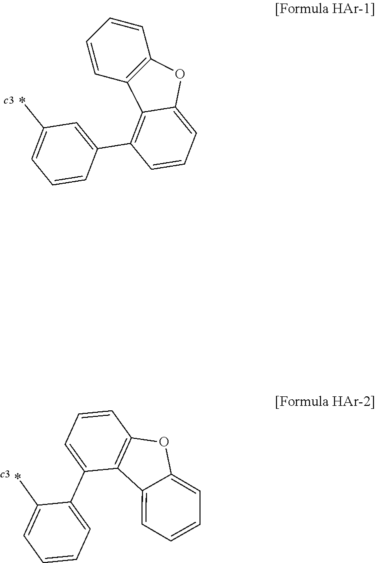

In an aspect, HAr represented by Formula 2 may be linked to Formula 1 at the R₁₁ position, and HAr may be positioned para to the nitrogen atom of the amine compound. Alternatively, HAr may be linked to Formula 1 at the R₁₂ position, and HAr may be positioned ortho, meta, or para to the nitrogen atom of the amine compound. Alternatively, HAr may be linked to Formula 1 at the R₁₃ position, and HAr may be positioned ortho, meta, or para to the nitrogen atom of the amine compound. Alternatively, HAr may be linked to Formula 1 at the R₁₁ position, and HAr may be positioned para to the nitrogen atom of the amine compound. For example, HAr linked to the phenyl group of Formula 1 may be represented by any one of DBH-a to DBH-h below. However, aspects of the present disclosure are not limited thereto.

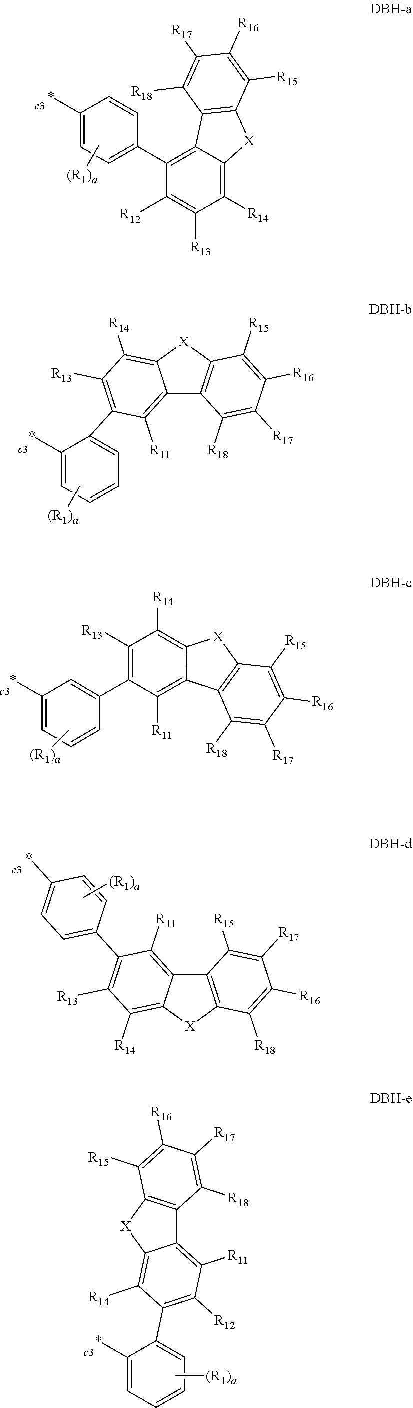

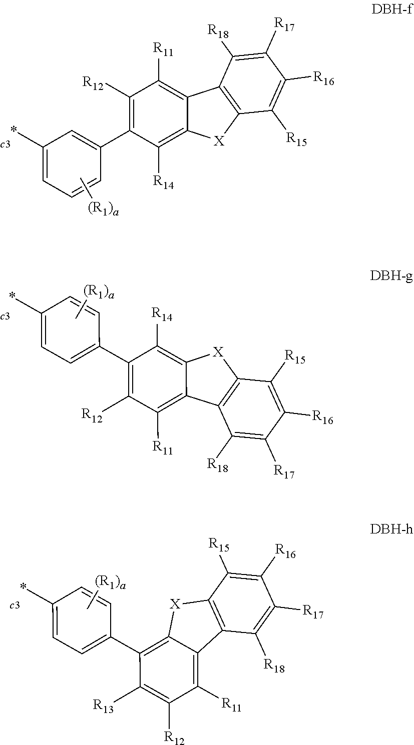

In Formulas DBH-a to DBH-h above, C3 is a position directly bonded to the nitrogen atom of the amine compound.

In Formulas DBH-a to DBH-h above, the description of Formula 2 may also apply to X, a, R₁, and R₁₁ to R₁₈.

However, when HAr is linked to Formula 1 at the R₁₂ position, a case in which R₁₅ is a phenyl group is excluded. That is, in the amine compound according to an aspect, when R₁₂ is linked to Formula 1, cases represented by Formulas HAr-3 to HAr-5 are excluded. In Formulas HAr-3 to HAr-5 below, C3 is a position directly bonded to the nitrogen atom of the amine compound. In Formulas HAr-3 to HAr-5, X is O or S.

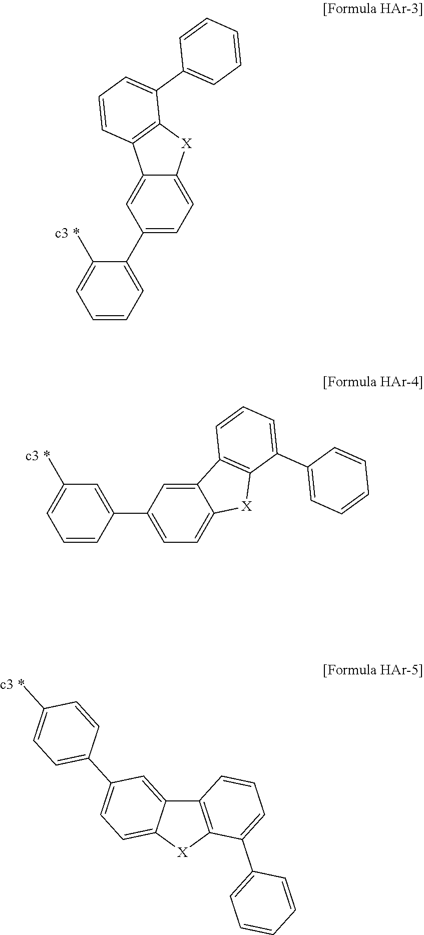

In an aspect, Formula 1 may be represented by any one of Formulas 1-a to 1-h below.

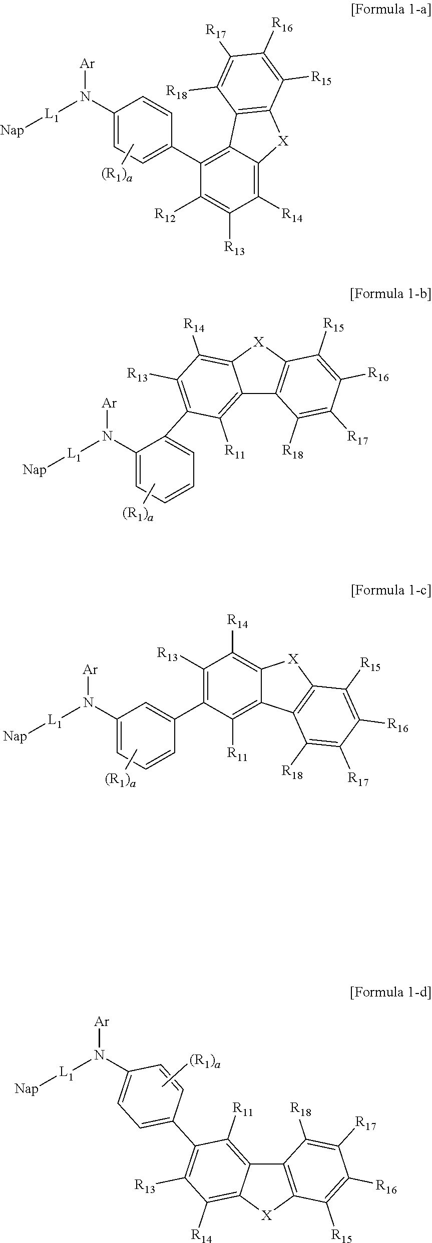

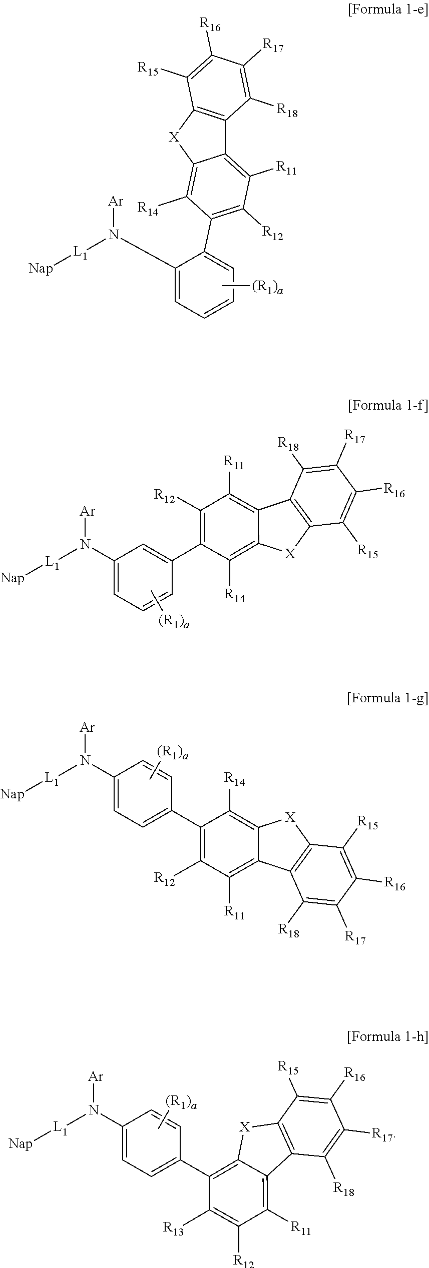

In Formulas 1-a to 1-h above, the description of Formula 2 may also apply to X, a, R₁, and R₁₁ to R₁₈. The description of Formula 1, which will be described later may also apply to Ar, L₁, and Nap.

In Formula 1, L₁ may be a direct linkage, a substituted or unsubstituted arylene group having 6 to 30 ring-forming carbon atoms excluding a substituted or unsubstituted naphthyl group, or a substituted or unsubstituted heteroarylene group having 5 to 30 ring-forming carbon atoms.

In an aspect, L₁ may be a direct linkage, a substituted or unsubstituted divalent phenyl, a substituted or unsubstituted divalent biphenyl, a substituted or unsubstituted divalent phenanthrene, a substituted or unsubstituted divalent triphenylene, a substituted or unsubstituted divalent fluorene, a substituted or unsubstituted divalent carbazole, a substituted or unsubstituted divalent dibenzothiophene, or a substituted or unsubstituted divalent dibenzofuran.

In Formula 1, Nap may be a substituted or unsubstituted phenylnaphthyl group.

In an aspect, Nap may be represented by Formula 3 or Formula 4 below.

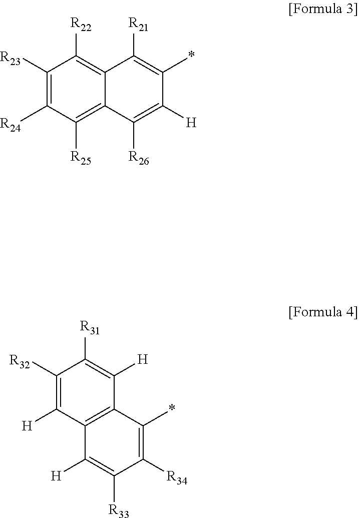

In Formula 3, any one of R₂₁ to R₂₆ may be a substituted or unsubstituted phenyl group, and the others may be a substituted or unsubstituted alkyl group having 1 to 15 carbon atoms, a halogen atom, a hydrogen atom, or a deuterium atom. However, when HAr is linked to Formula 1 at the R₁₄ position and when L₁ is a direct linkage, cases in which R₂₁ or R₂₄ is a substituted or unsubstituted phenyl group are excluded. That is, in the amine compound according to an aspect, when R₁₄ of HAr is linked to a phenyl group directly bonded to a nitrogen atom and when L¹ is a direct linkage, a case in which Nap is represented by Formulas Nap-1 and Nap-2 below is excluded.

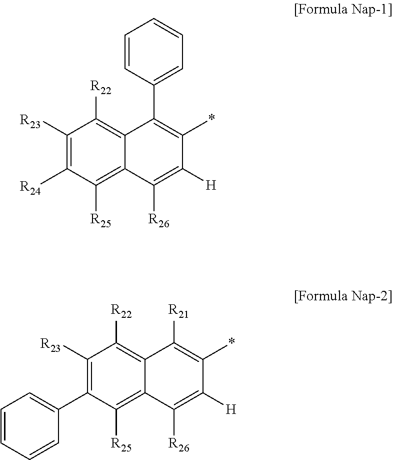

In Formula 4, any one of R₃₁ to R₃₄ may be a substituted or unsubstituted phenyl group, and the others may each independently be a substituted or unsubstituted alkyl group having 1 to 15 carbon atoms, a halogen atom, a hydrogen atom, or a deuterium atom. However, when HAr is linked to Formula 1 at the R₁₄ position, a case in which R₃₄ is a substituted or unsubstituted phenyl group is excluded. That is, in the amine compound according to an aspect, when HAr is linked to a phenyl group directly bonded to the nitrogen atom at the R₁₄ position, a case in which Nap is represented by Formula Nap-3 below is excluded.

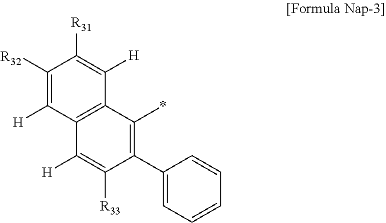

Formula 1 may be represented by Formula 1-1 or Formula 1-2 below. Formula 1-1 shows an amine compound according to an aspect in which a phenylnaphthyl group (Nap) is directly bonded to a nitrogen atom, and Formula 1-2 shows an amine compound according to an aspect in which a phenylnaphthyl group (Nap) is bonded to a nitrogen atom through a linker L₂.

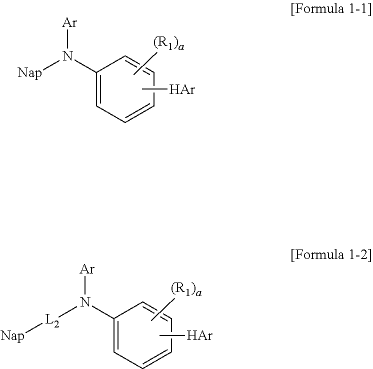

In Formula 1-2 above, L₂ may be a substituted or unsubstituted divalent phenyl, a substituted or unsubstituted divalent biphenyl, or a substituted or unsubstituted divalent dibenzofuran. For example, L₂ may be represented by any one of L-a to L-d below. However, aspects of the present disclosure are not limited thereto.

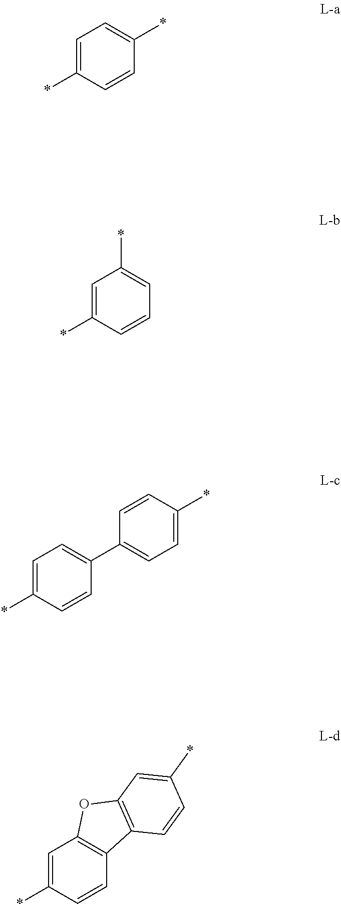 [SMILES: *c1ccc(*)cc1 ; *c1cccc(*)c1 ; *c1ccc(-c2ccc(*)cc2)cc1 ; *c1ccc2c(c1)oc1cc(*)ccc12]

In Formulas 1-1 and 1-2 above, the description of Formula 1 may also apply to Nap, Ar, HAr, R₁, and a.

For example, L₂ may be an unsubstituted divalent phenyl.

The amine compound according to an aspect represented by Formula 1 may be represented by any one of compounds from Compound Group 1 below. The hole transport region HTR of the light emitting element ED according to an aspect may include at least one of the amine compounds disclosed in Compound Group 1 below. In Compound Group 1 below, D is a deuterium atom.

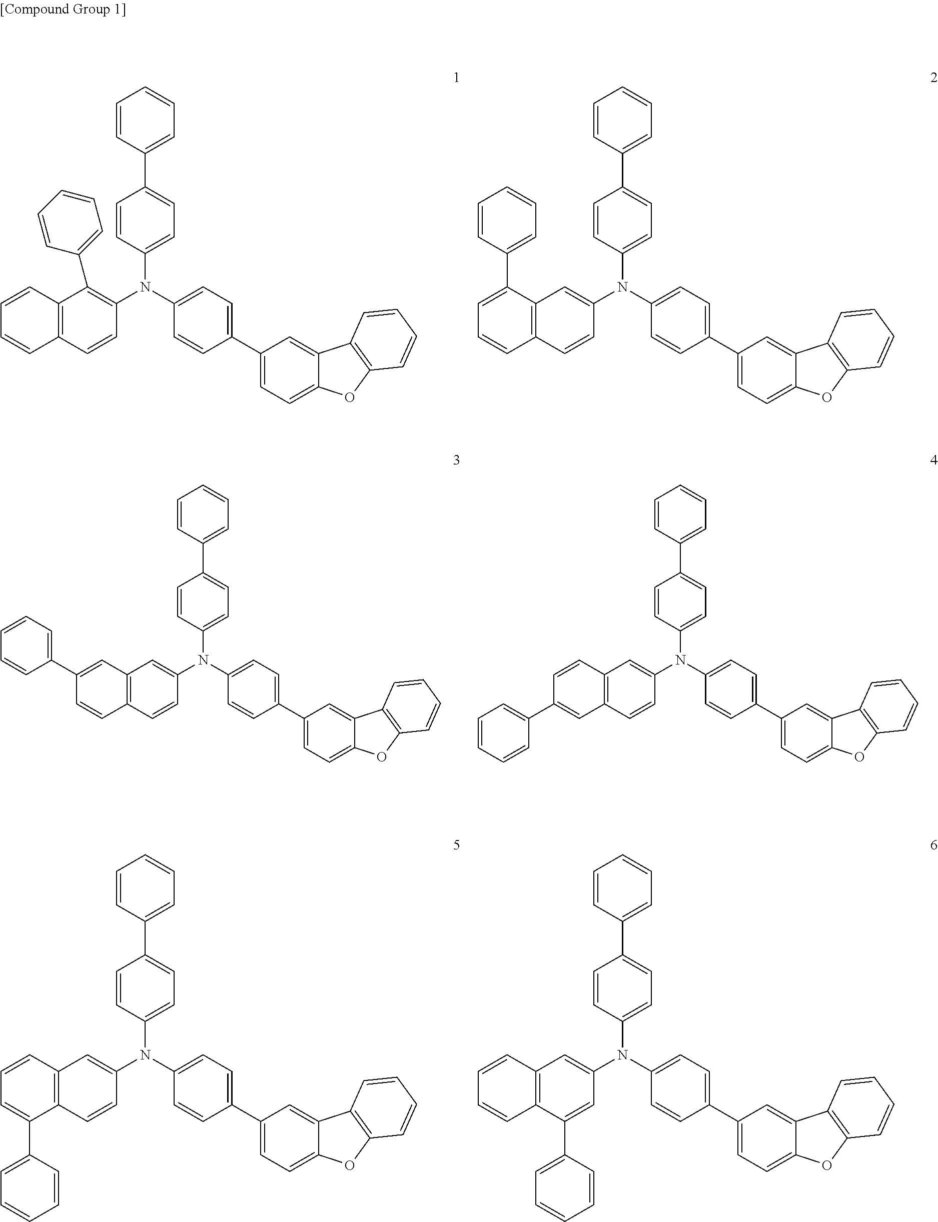

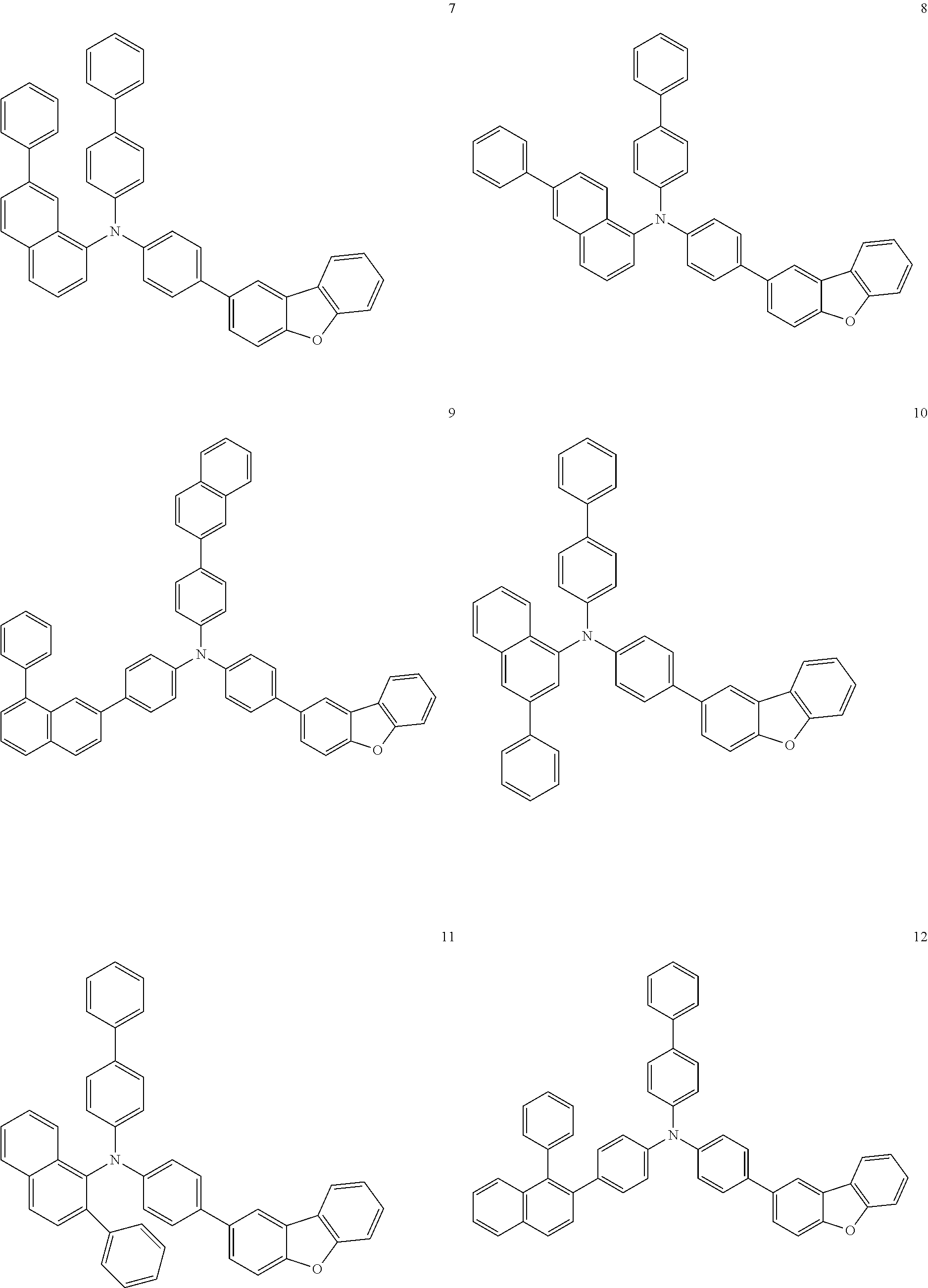

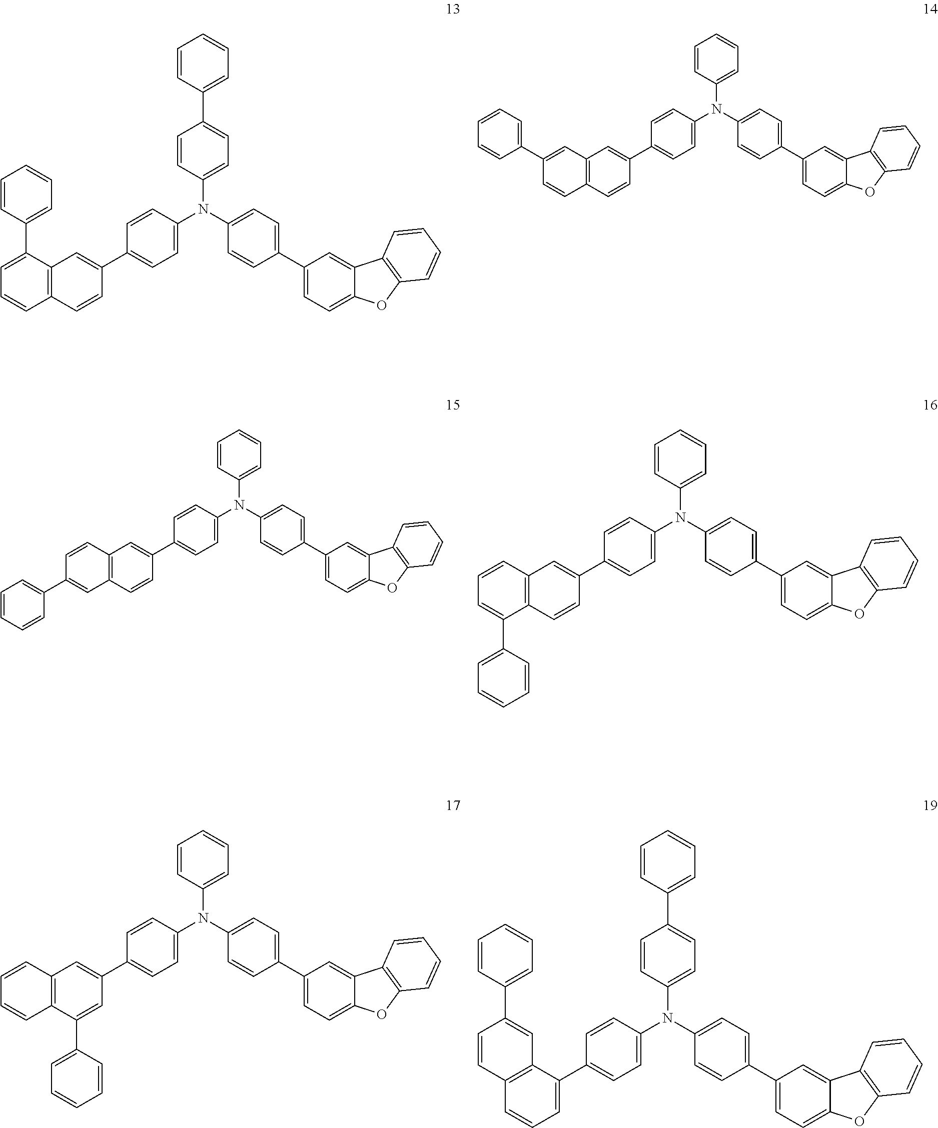

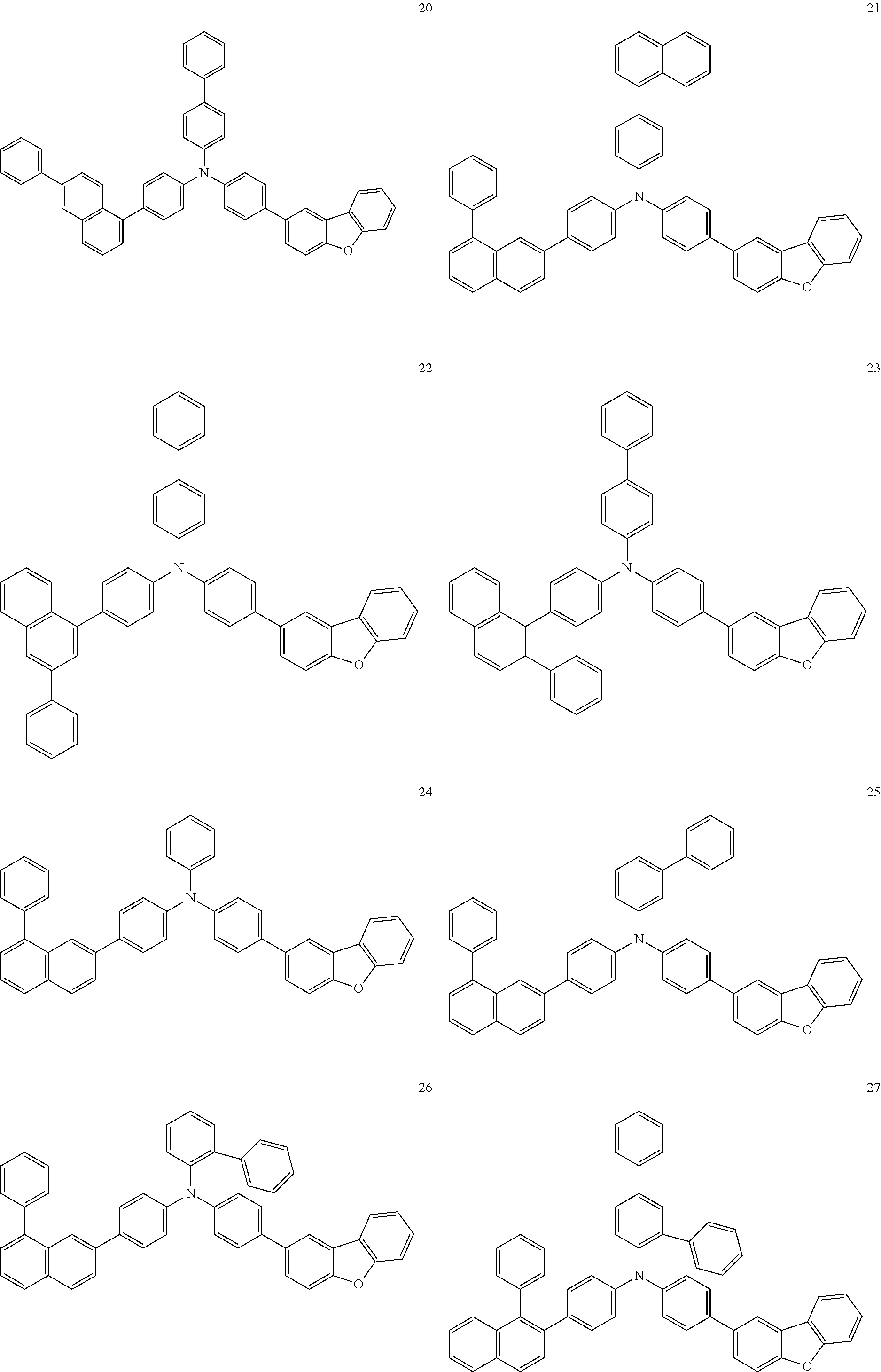

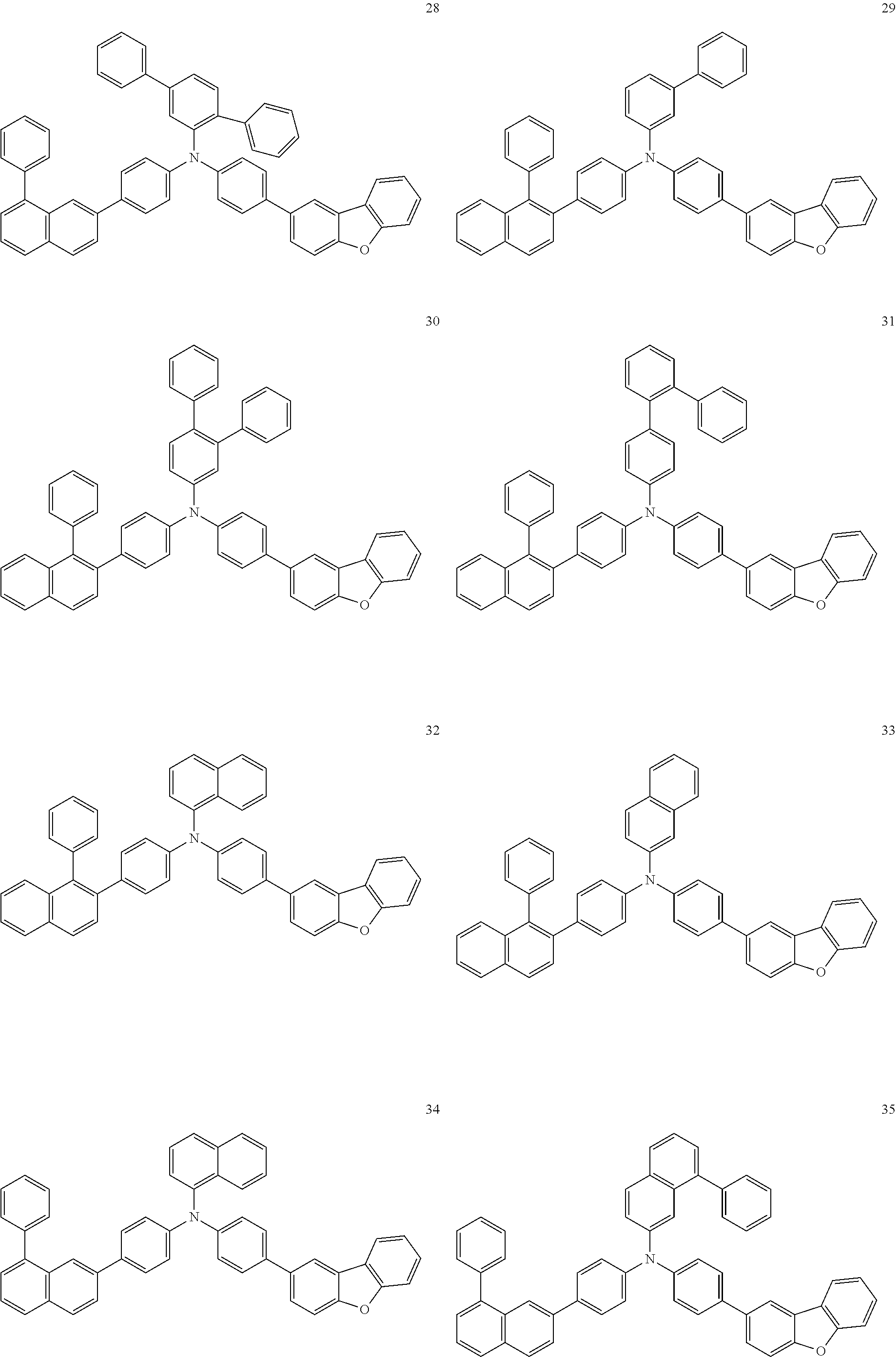

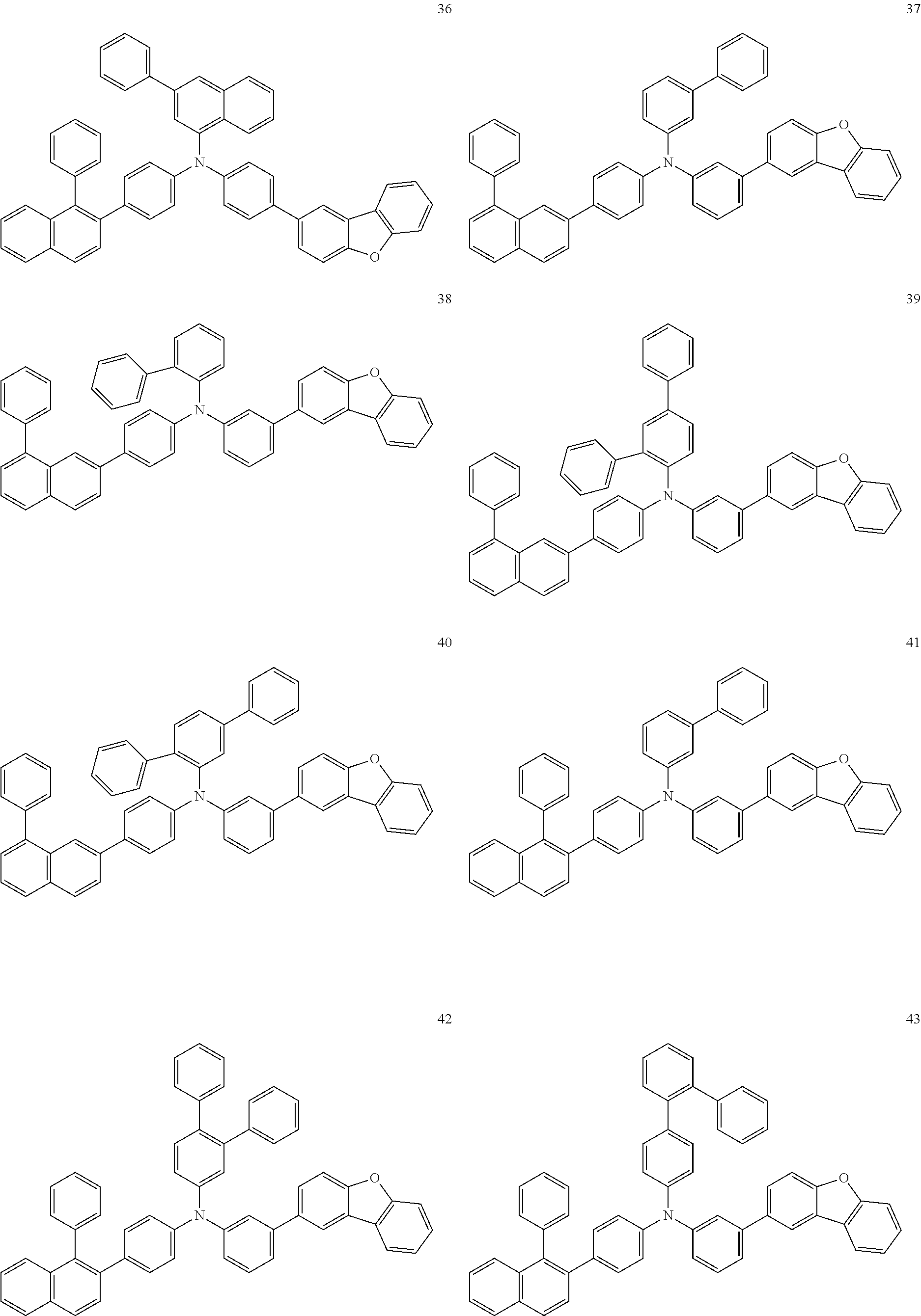

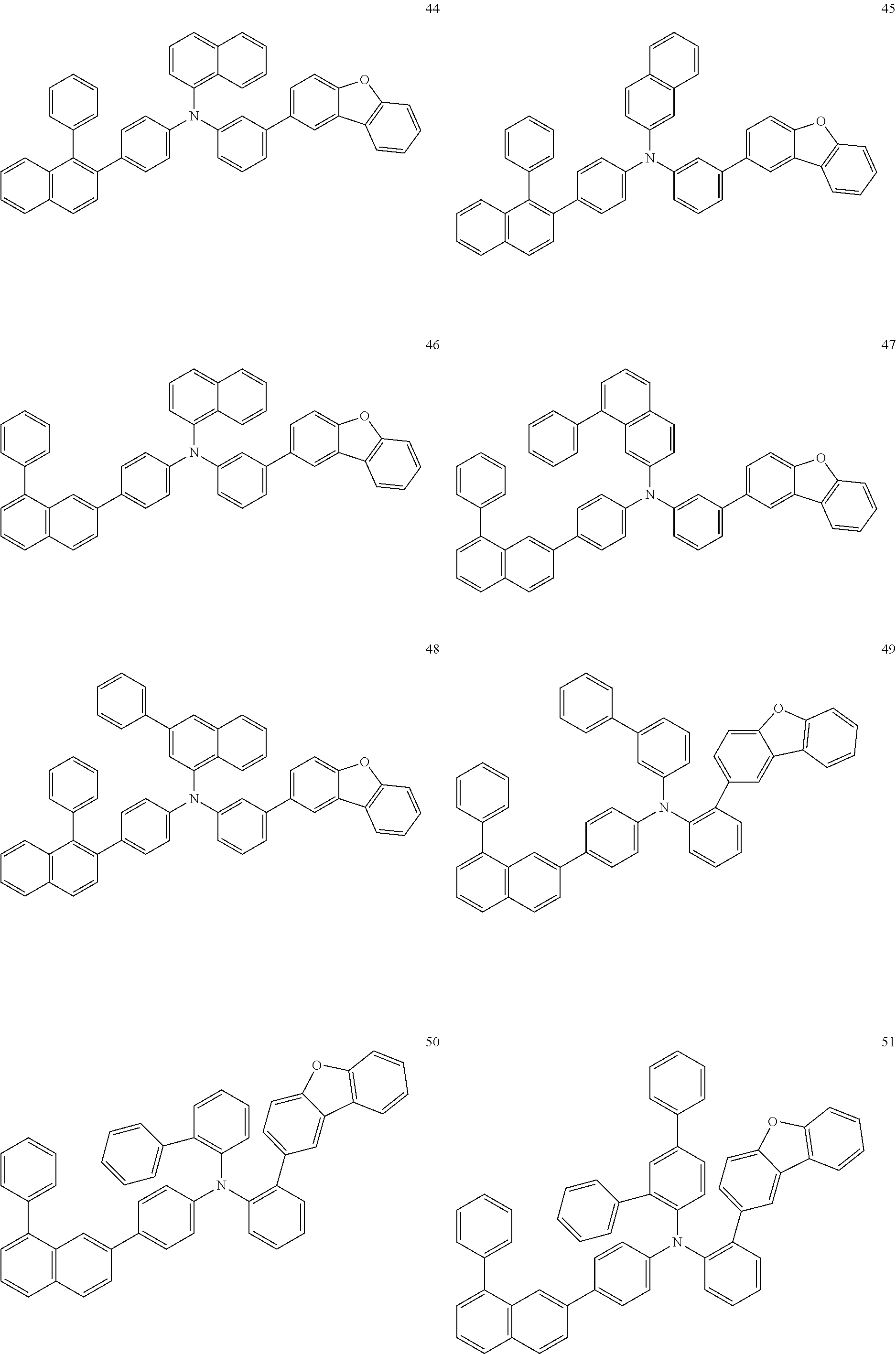

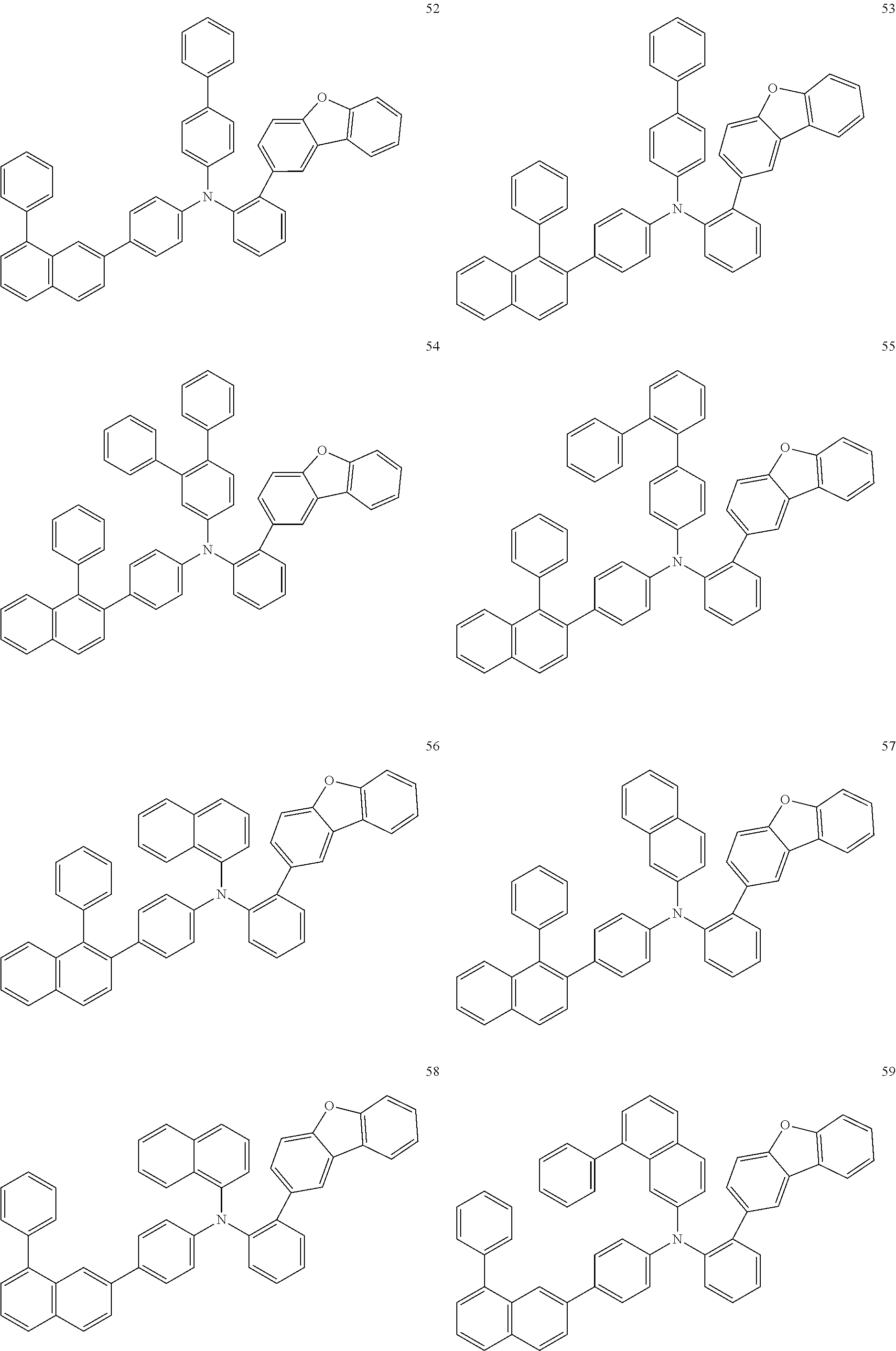

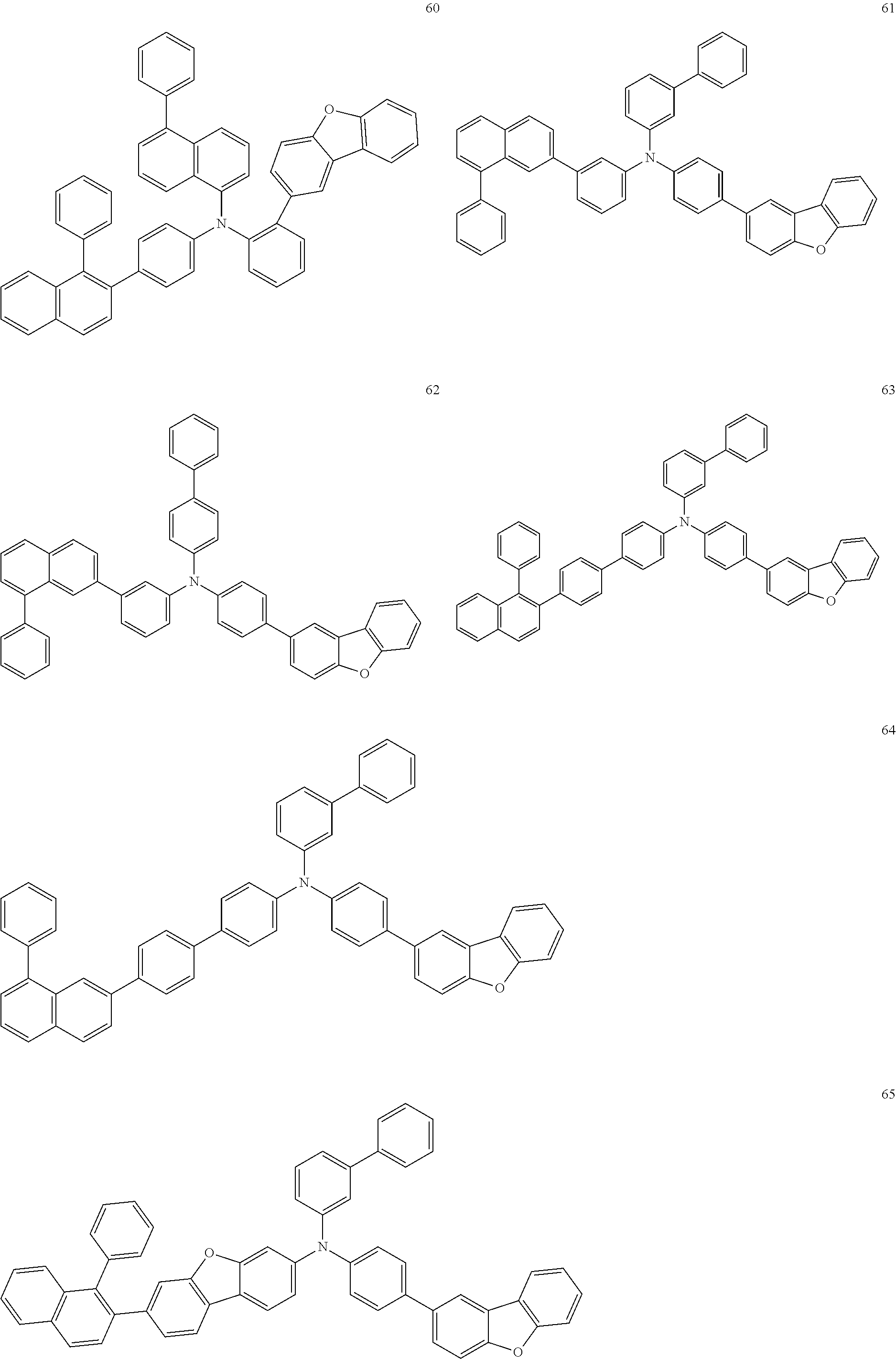

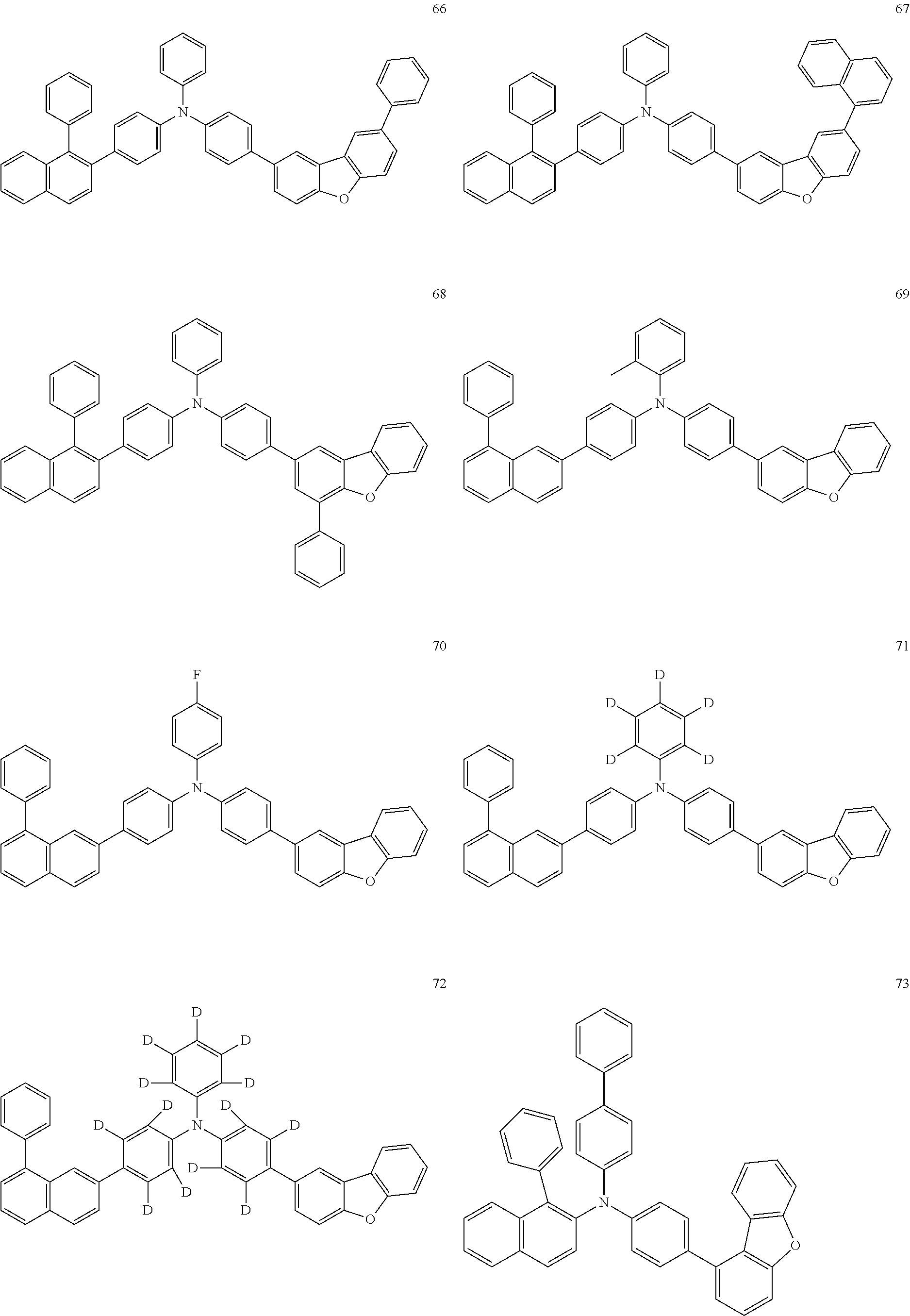

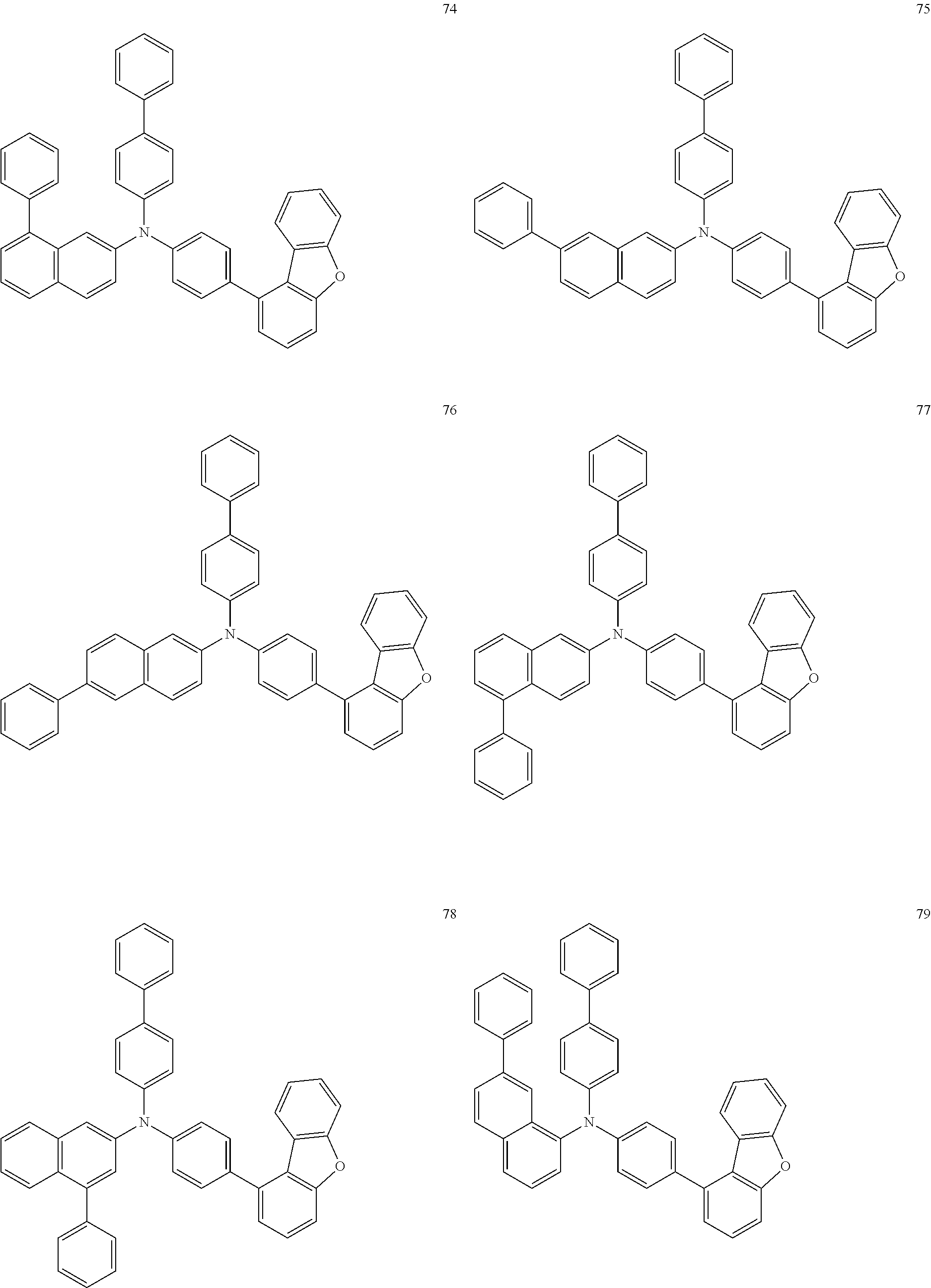 [SMILES: c1ccc(-c2ccc(N(c3ccc(-c4cccc5oc6ccccc6c45)cc3)c3ccc4cccc(-c5ccccc5)c4c3)cc2)cc1 ; c1ccc(-c2ccc(N(c3ccc(-c4cccc5oc6ccccc6c45)cc3)c3ccc4ccc(-c5ccccc5)cc4c3)cc2)cc1 ; c1ccc(-c2ccc(N(c3ccc(-c4cccc5oc6ccccc6c45)cc3)c3ccc4cc(-c5ccccc5)ccc4c3)cc2)cc1 ; c1ccc(-c2ccc(N(c3ccc(-c4cccc5oc6ccccc6c45)cc3)c3ccc4c(-c5ccccc5)cccc4c3)cc2)cc1 ; c1ccc(-c2ccc(N(c3ccc(-c4cccc5oc6ccccc6c45)cc3)c3cc(-c4ccccc4)c4ccccc4c3)cc2)cc1 ; c1ccc(-c2ccc(N(c3ccc(-c4cccc5oc6ccccc6c45)cc3)c3cccc4ccc(-c5ccccc5)cc34)cc2)cc1]

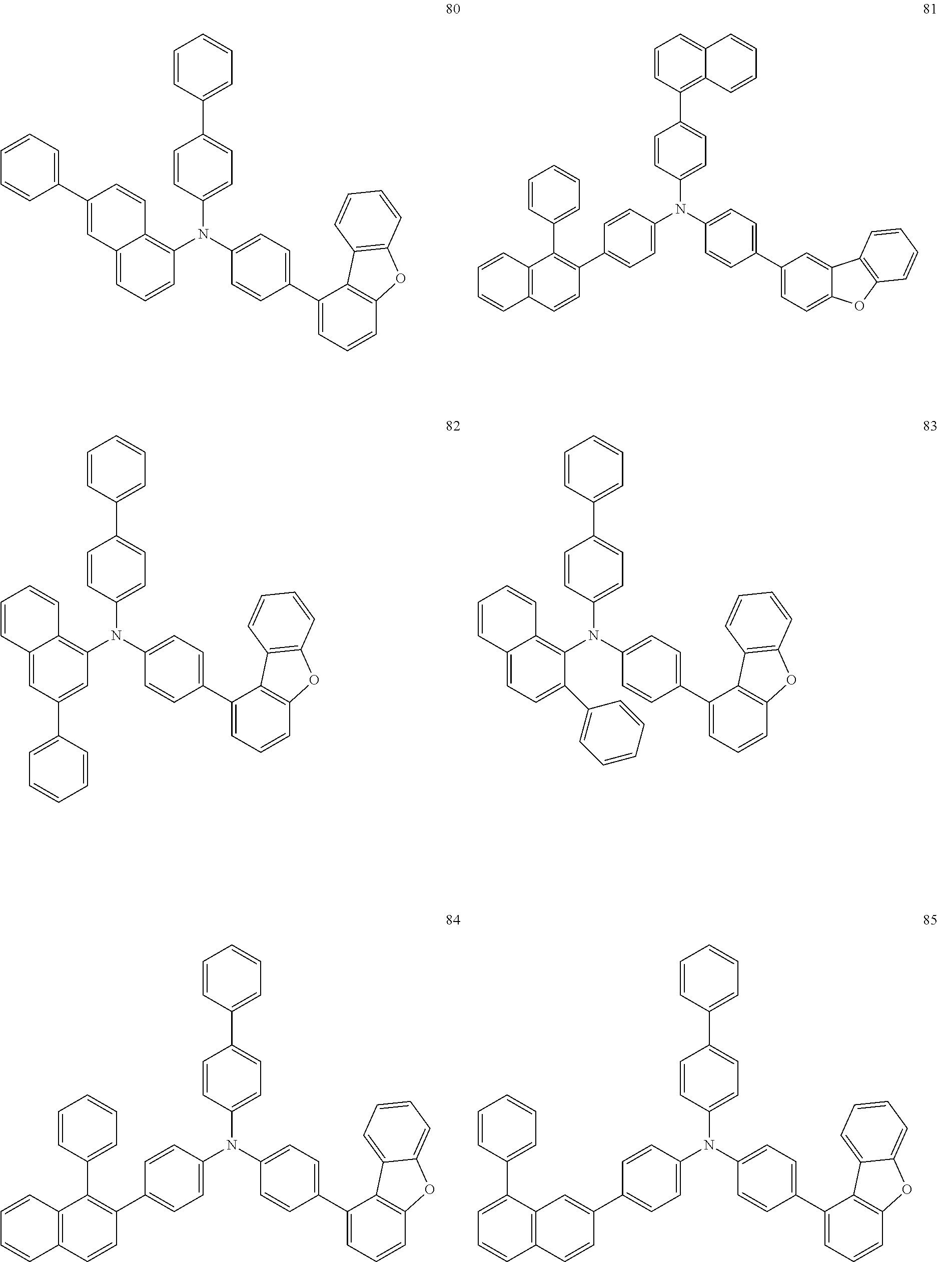

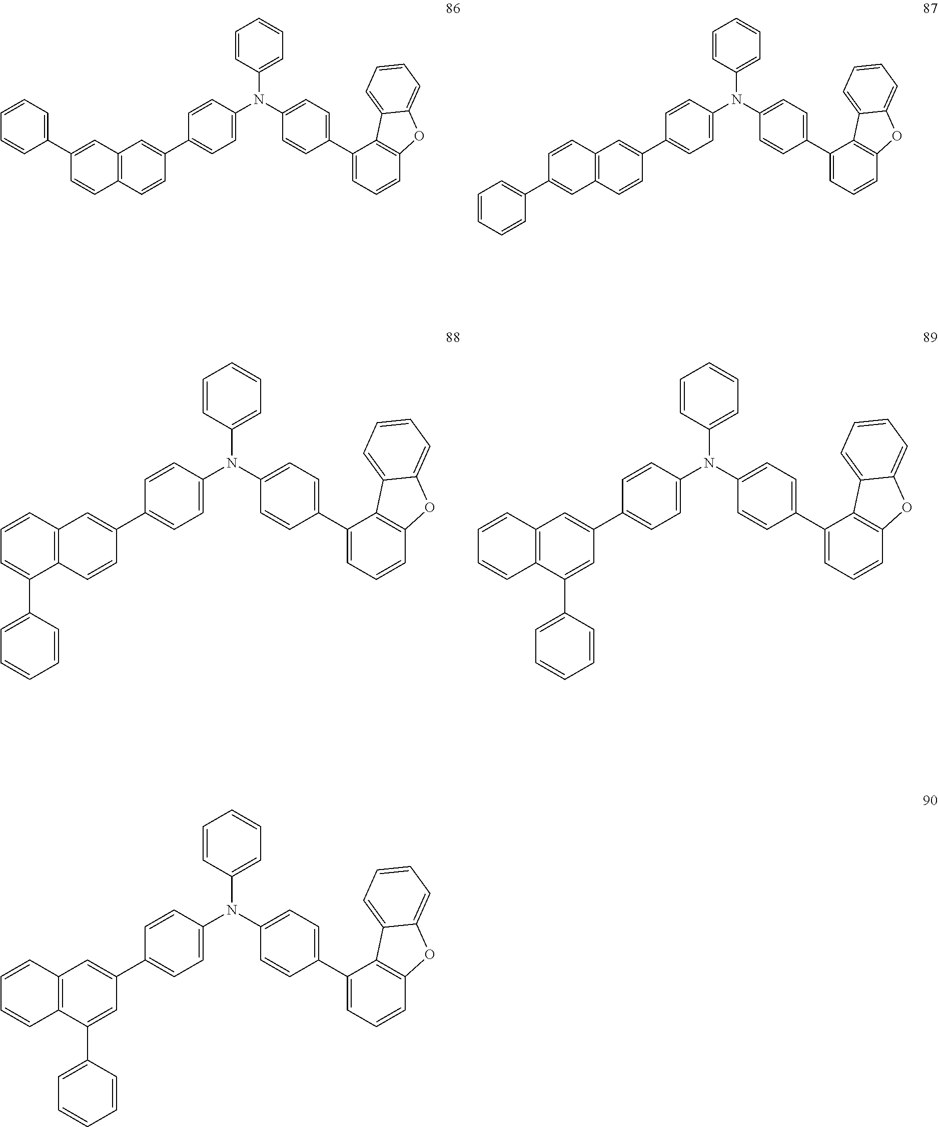 [SMILES: c1ccc(-c2ccc3ccc(-c4ccc(N(c5ccccc5)c5ccc(-c6cccc7oc8ccccc8c67)cc5)cc4)cc3c2)cc1 ; c1ccc(-c2ccc3cc(-c4ccc(N(c5ccccc5)c5ccc(-c6cccc7oc8ccccc8c67)cc5)cc4)ccc3c2)cc1 ; c1ccc(-c2cccc3cc(-c4ccc(N(c5ccccc5)c5ccc(-c6cccc7oc8ccccc8c67)cc5)cc4)ccc23)cc1 ; c1ccc(-c2cc(-c3ccc(N(c4ccccc4)c4ccc(-c5cccc6oc7ccccc7c56)cc4)cc3)cc3ccccc23)cc1 ; c1ccc(-c2cc(-c3ccc(N(c4ccccc4)c4ccc(-c5cccc6oc7ccccc7c56)cc4)cc3)cc3ccccc23)cc1]

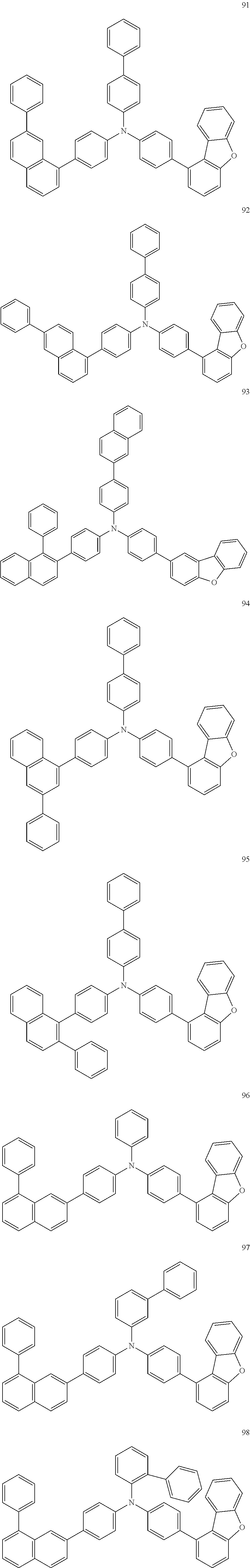

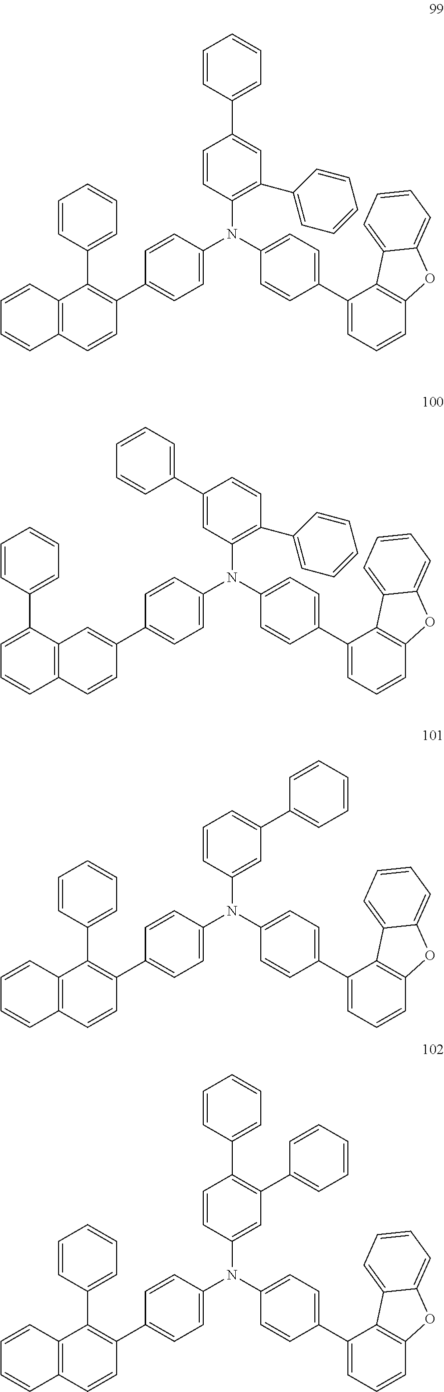 [SMILES: c1ccc(-c2ccc(N(c3ccc(-c4ccc5ccccc5c4-c4ccccc4)cc3)c3ccc(-c4cccc5oc6ccccc6c45)cc3)c(-c3ccccc3)c2)cc1 ; c1ccc(-c2ccc(-c3ccccc3)c(N(c3ccc(-c4ccc5cccc(-c6ccccc6)c5c4)cc3)c3ccc(-c4cccc5oc6ccccc6c45)cc3)c2)cc1 ; c1ccc(-c2cccc(N(c3ccc(-c4ccc5ccccc5c4-c4ccccc4)cc3)c3ccc(-c4cccc5oc6ccccc6c45)cc3)c2)cc1 ; c1ccc(-c2ccc(N(c3ccc(-c4ccc5ccccc5c4-c4ccccc4)cc3)c3ccc(-c4cccc5oc6ccccc6c45)cc3)cc2-c2ccccc2)cc1]

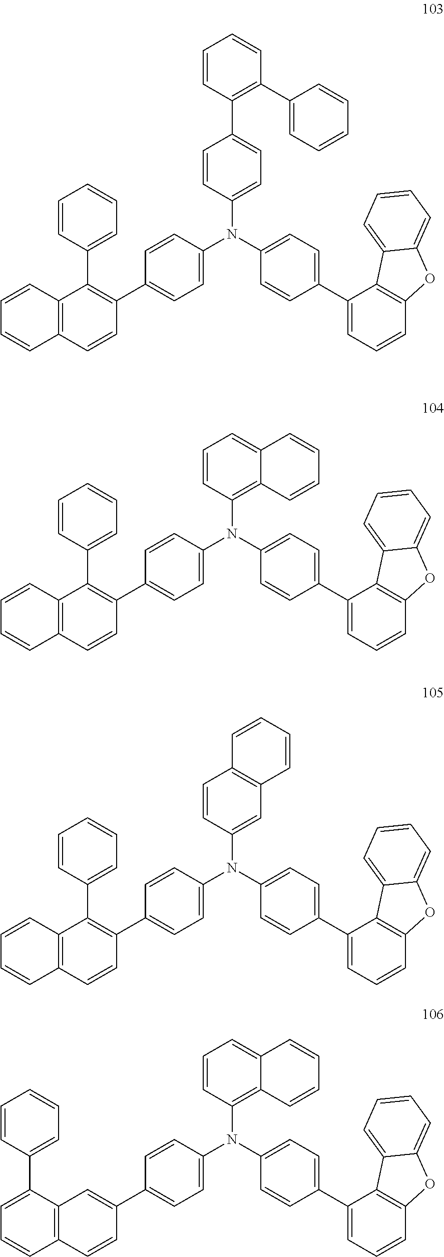 [SMILES: c1ccc(-c2ccccc2-c2ccc(N(c3ccc(-c4ccc5ccccc5c4-c4ccccc4)cc3)c3ccc(-c4cccc5oc6ccccc6c45)cc3)cc2)cc1 ; c1ccc(-c2c(-c3ccc(N(c4ccc(-c5cccc6oc7ccccc7c56)cc4)c4cccc5ccccc45)cc3)ccc3ccccc23)cc1 ; c1ccc(-c2c(-c3ccc(N(c4ccc(-c5cccc6oc7ccccc7c56)cc4)c4ccc5ccccc5c4)cc3)ccc3ccccc23)cc1 ; c1ccc(-c2cccc3ccc(-c4ccc(N(c5ccc(-c6cccc7oc8ccccc8c67)cc5)c5cccc6ccccc56)cc4)cc23)cc1]

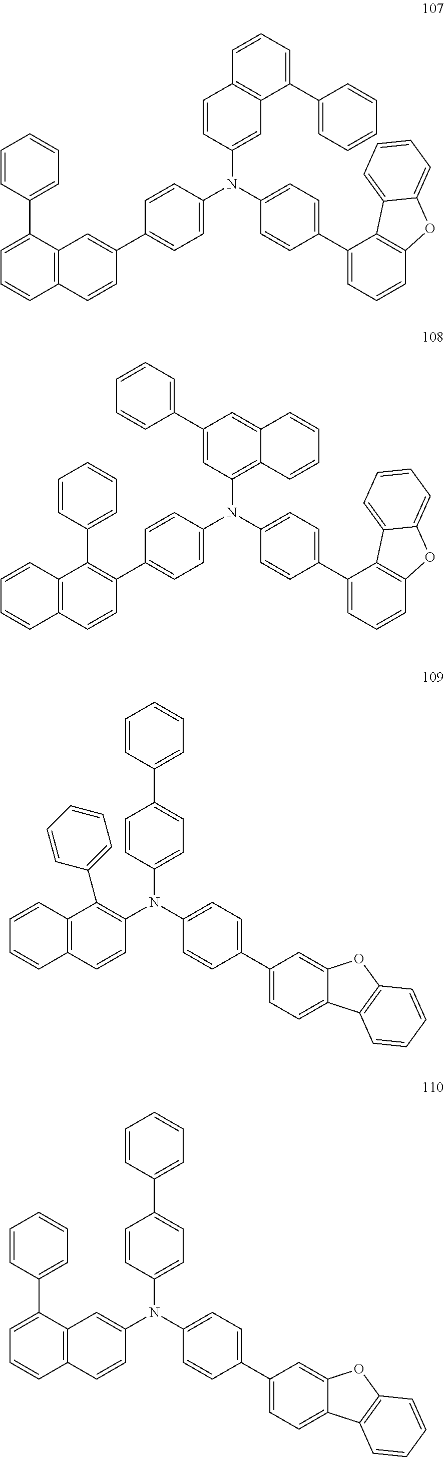 [SMILES: c1ccc(-c2cccc3ccc(-c4ccc(N(c5ccc(-c6cccc7oc8ccccc8c67)cc5)c5ccc6cccc(-c7ccccc7)c6c5)cc4)cc23)cc1 ; c1ccc(-c2cc(N(c3ccc(-c4ccc5ccccc5c4-c4ccccc4)cc3)c3ccc(-c4cccc5oc6ccccc6c45)cc3)c3ccccc3c2)cc1 ; c1ccc(-c2ccc(N(c3ccc(-c4ccc5c(c4)oc4ccccc45)cc3)c3ccc4ccccc4c3-c3ccccc3)cc2)cc1 ; c1ccc(-c2ccc(N(c3ccc(-c4ccc5c(c4)oc4ccccc45)cc3)c3ccc4cccc(-c5ccccc5)c4c3)cc2)cc1]

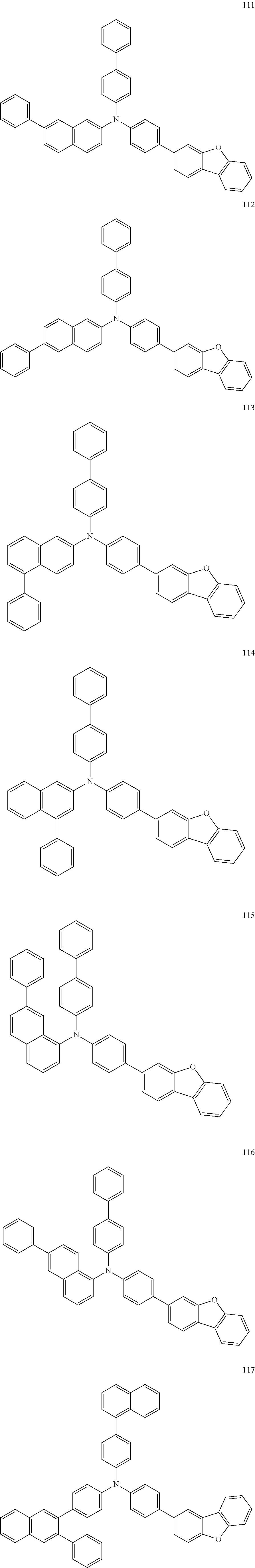

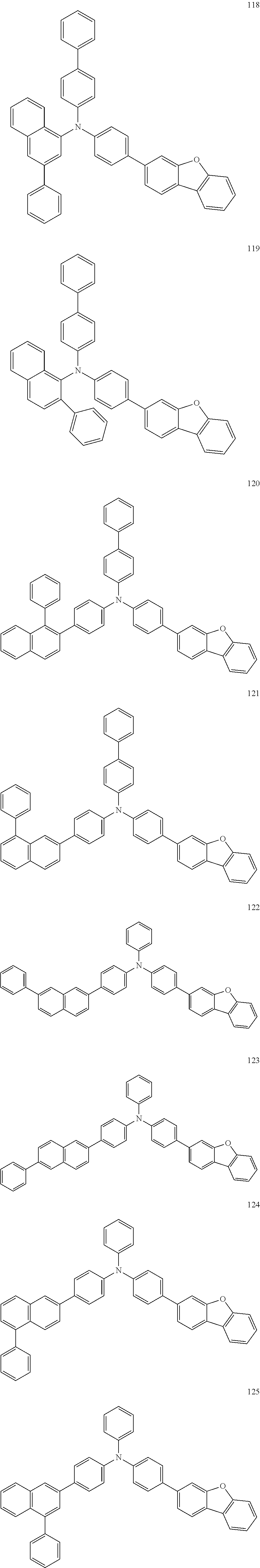

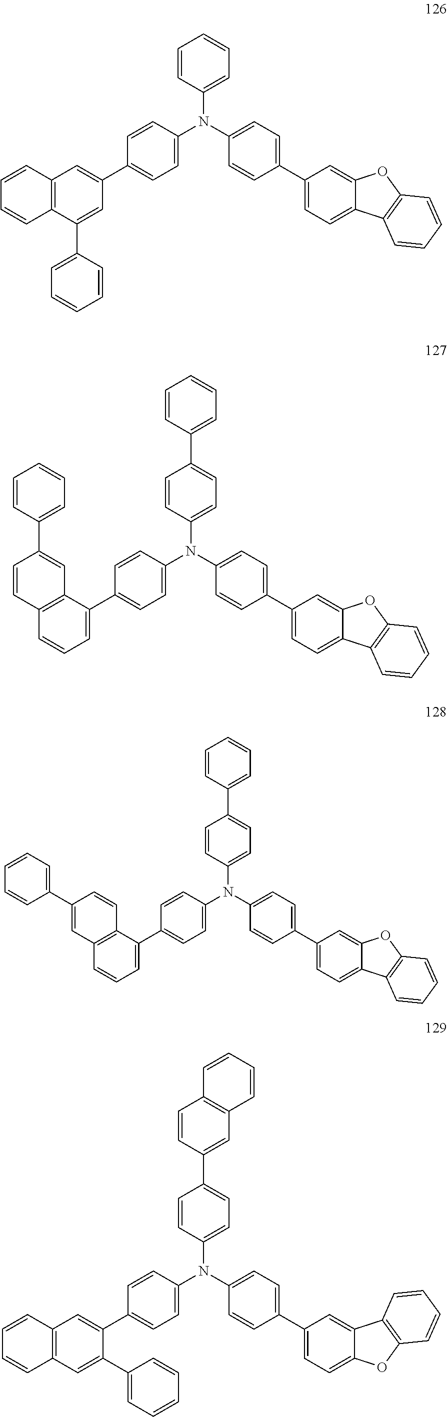 [SMILES: c1ccc(-c2cc(-c3ccc(N(c4ccccc4)c4ccc(-c5ccc6c(c5)oc5ccccc56)cc4)cc3)cc3ccccc23)cc1 ; c1ccc(-c2ccc(N(c3ccc(-c4ccc5c(c4)oc4ccccc45)cc3)c3ccc(-c4cccc5ccc(-c6ccccc6)cc45)cc3)cc2)cc1 ; c1ccc(-c2ccc(N(c3ccc(-c4ccc5c(c4)oc4ccccc45)cc3)c3ccc(-c4cccc5cc(-c6ccccc6)ccc45)cc3)cc2)cc1 ; c1ccc(-c2cc3ccccc3cc2-c2ccc(N(c3ccc(-c4ccc5ccccc5c4)cc3)c3ccc(-c4ccc5oc6ccccc6c5c4)cc3)cc2)cc1]

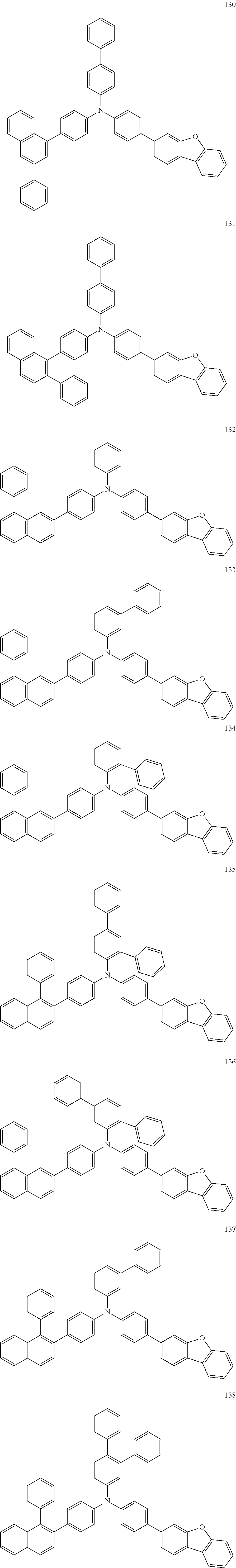

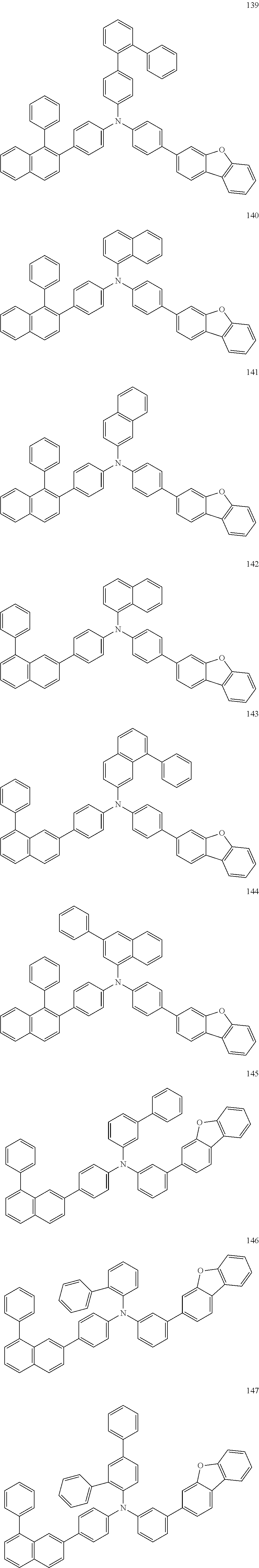

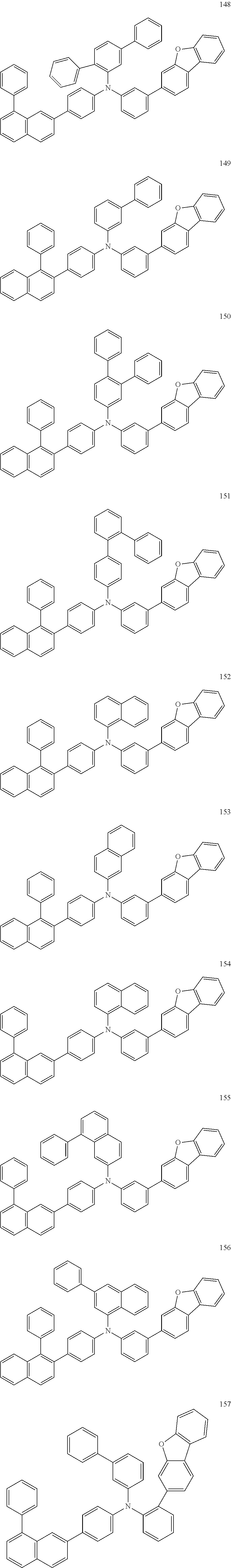

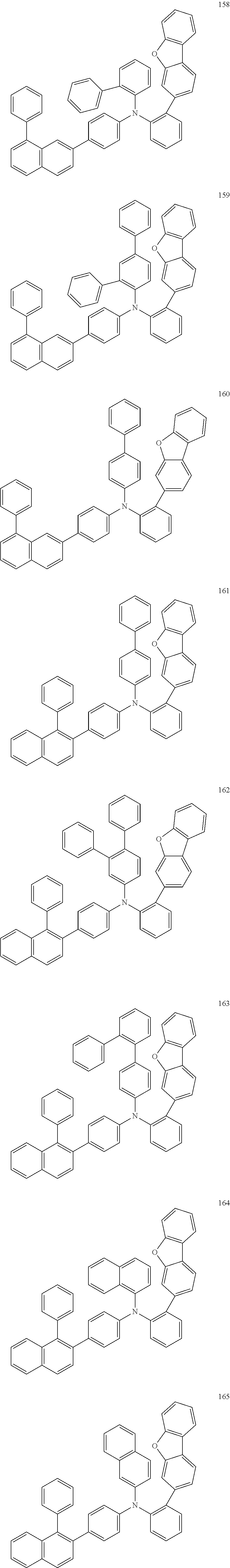

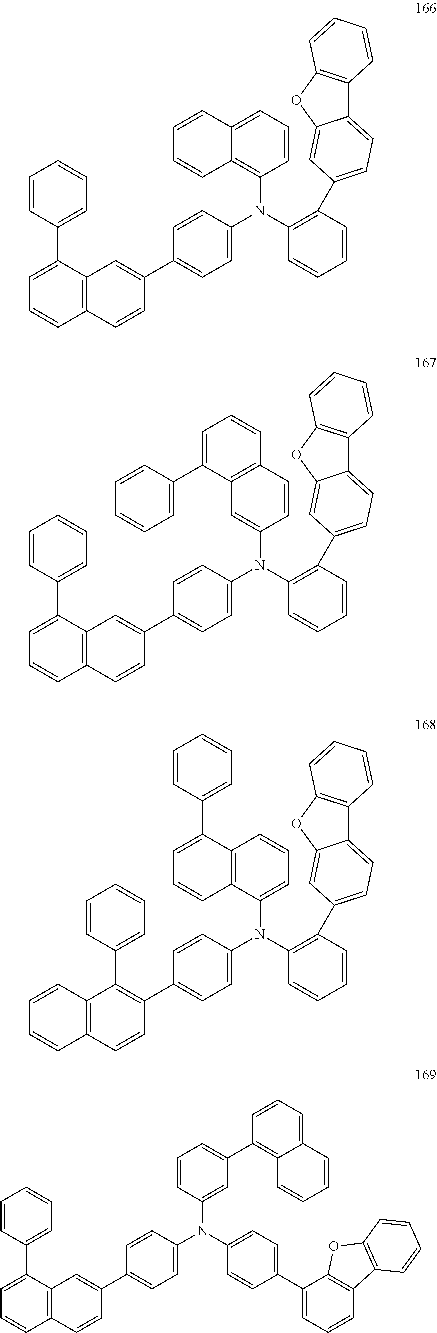 [SMILES: c1ccc(-c2cccc3ccc(-c4ccc(N(c5ccccc5-c5ccc6c(c5)oc5ccccc56)c5cccc6ccccc56)cc4)cc23)cc1 ; c1ccc(-c2cccc3ccc(-c4ccc(N(c5ccc6cccc(-c7ccccc7)c6c5)c5ccccc5-c5ccc6c(c5)oc5ccccc56)cc4)cc23)cc1 ; c1ccc(-c2c(-c3ccc(N(c4ccccc4-c4ccc5c(c4)oc4ccccc45)c4cccc5c(-c6ccccc6)cccc45)cc3)ccc3ccccc23)cc1 ; c1ccc(-c2cccc3ccc(-c4ccc(N(c5ccc(-c6cccc7c6oc6ccccc67)cc5)c5cccc(-c6cccc7ccccc67)c5)cc4)cc23)cc1]

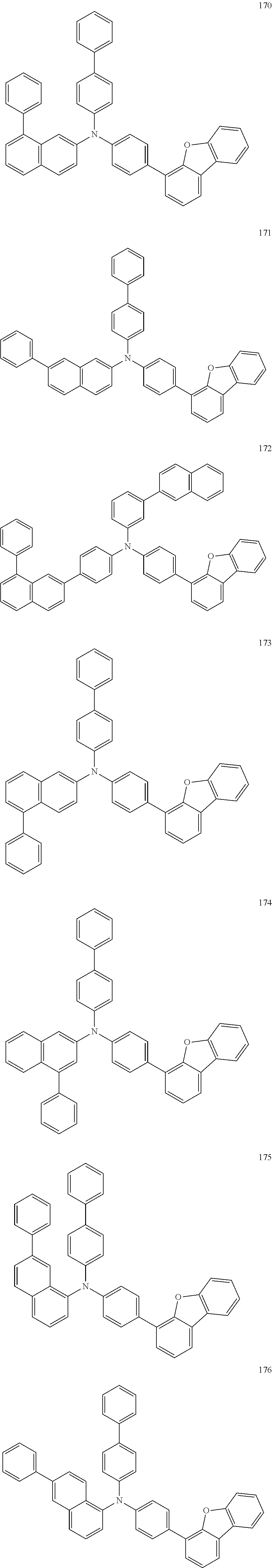

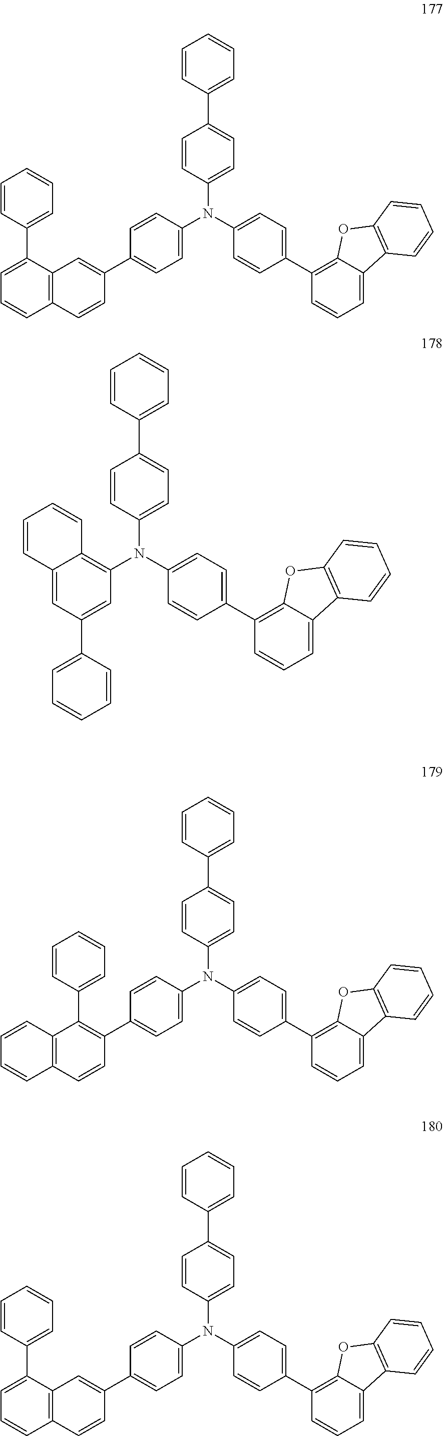 [SMILES: c1ccc(-c2ccc(N(c3ccc(-c4ccc5cccc(-c6ccccc6)c5c4)cc3)c3ccc(-c4cccc5c4oc4ccccc45)cc3)cc2)cc1 ; c1ccc(-c2ccc(N(c3ccc(-c4cccc5c4oc4ccccc45)cc3)c3cc(-c4ccccc4)cc4ccccc34)cc2)cc1 ; c1ccc(-c2ccc(N(c3ccc(-c4ccc5ccccc5c4-c4ccccc4)cc3)c3ccc(-c4cccc5c4oc4ccccc45)cc3)cc2)cc1 ; c1ccc(-c2ccc(N(c3ccc(-c4ccc5cccc(-c6ccccc6)c5c4)cc3)c3ccc(-c4cccc5c4oc4ccccc45)cc3)cc2)cc1]

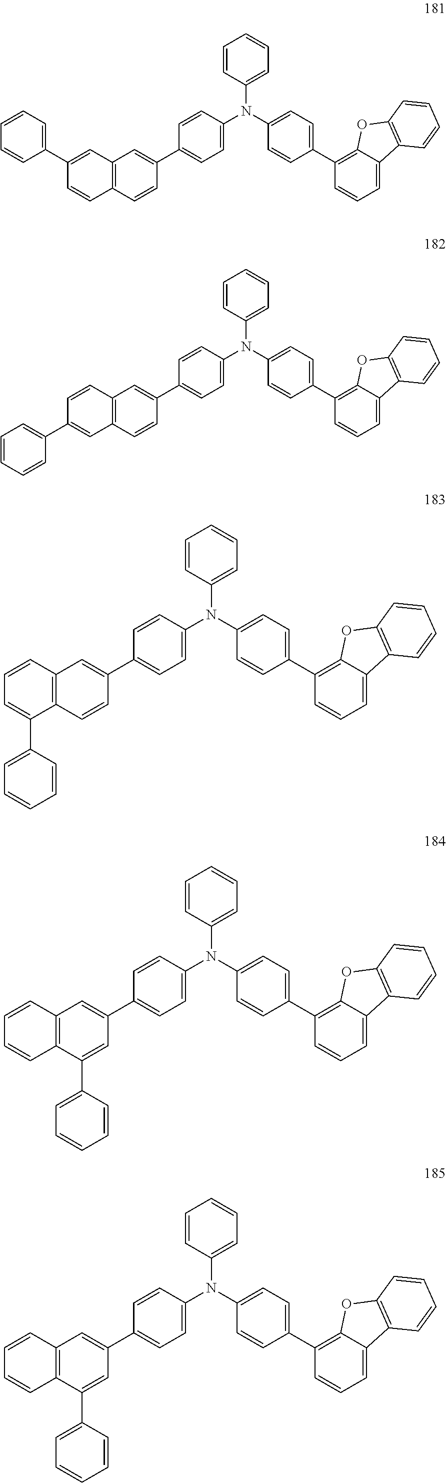 [SMILES: c1ccc(-c2ccc3ccc(-c4ccc(N(c5ccccc5)c5ccc(-c6cccc7c6oc6ccccc67)cc5)cc4)cc3c2)cc1 ; c1ccc(-c2ccc3cc(-c4ccc(N(c5ccccc5)c5ccc(-c6cccc7c6oc6ccccc67)cc5)cc4)ccc3c2)cc1 ; c1ccc(-c2cccc3cc(-c4ccc(N(c5ccccc5)c5ccc(-c6cccc7c6oc6ccccc67)cc5)cc4)ccc23)cc1 ; c1ccc(-c2cc(-c3ccc(N(c4ccccc4)c4ccc(-c5cccc6c5oc5ccccc56)cc4)cc3)cc3ccccc23)cc1 ; c1ccc(-c2cc(-c3ccc(N(c4ccccc4)c4ccc(-c5cccc6c5oc5ccccc56)cc4)cc3)cc3ccccc23)cc1]

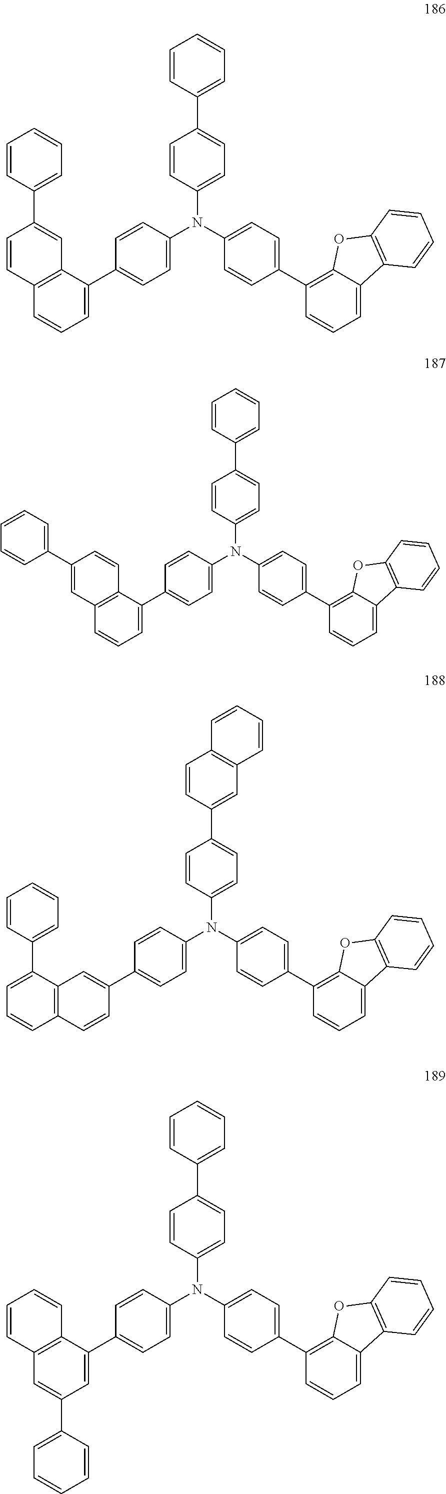 [SMILES: c1ccc(-c2ccc(N(c3ccc(-c4cccc5ccc(-c6ccccc6)cc45)cc3)c3ccc(-c4cccc5c4oc4ccccc45)cc3)cc2)cc1 ; c1ccc(-c2ccc(N(c3ccc(-c4cccc5cc(-c6ccccc6)ccc45)cc3)c3ccc(-c4cccc5c4oc4ccccc45)cc3)cc2)cc1 ; c1ccc(-c2cccc3ccc(-c4ccc(N(c5ccc(-c6ccc7ccccc7c6)cc5)c5ccc(-c6cccc7c6oc6ccccc67)cc5)cc4)cc23)cc1 ; c1ccc(-c2ccc(N(c3ccc(-c4cc(-c5ccccc5)cc5ccccc45)cc3)c3ccc(-c4cccc5c4oc4ccccc45)cc3)cc2)cc1]

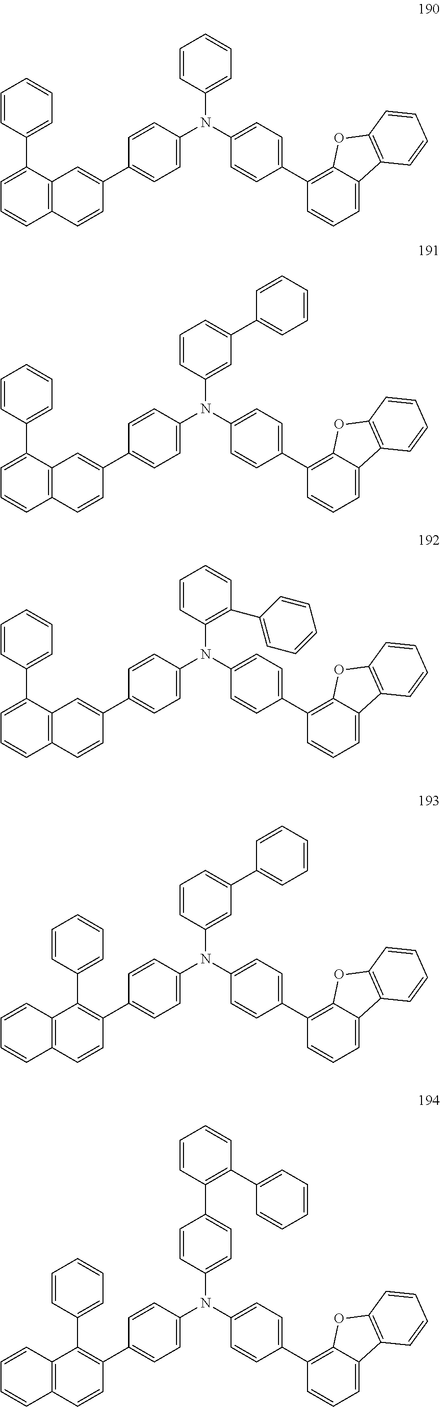 [SMILES: c1ccc(-c2cccc3ccc(-c4ccc(N(c5ccccc5)c5ccc(-c6cccc7c6oc6ccccc67)cc5)cc4)cc23)cc1 ; c1ccc(-c2cccc(N(c3ccc(-c4ccc5cccc(-c6ccccc6)c5c4)cc3)c3ccc(-c4cccc5c4oc4ccccc45)cc3)c2)cc1 ; c1ccc(-c2ccccc2N(c2ccc(-c3ccc4cccc(-c5ccccc5)c4c3)cc2)c2ccc(-c3cccc4c3oc3ccccc34)cc2)cc1 ; c1ccc(-c2cccc(N(c3ccc(-c4ccc5ccccc5c4-c4ccccc4)cc3)c3ccc(-c4cccc5c4oc4ccccc45)cc3)c2)cc1 ; c1ccc(-c2ccccc2-c2ccc(N(c3ccc(-c4ccc5ccccc5c4-c4ccccc4)cc3)c3ccc(-c4cccc5c4oc4ccccc45)cc3)cc2)cc1]

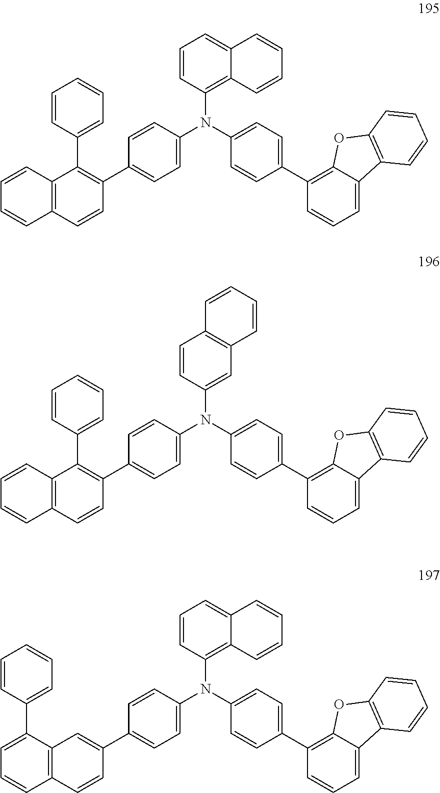 [SMILES: c1ccc(-c2c(-c3ccc(N(c4ccc(-c5cccc6c5oc5ccccc56)cc4)c4cccc5ccccc45)cc3)ccc3ccccc23)cc1 ; c1ccc(-c2c(-c3ccc(N(c4ccc(-c5cccc6c5oc5ccccc56)cc4)c4ccc5ccccc5c4)cc3)ccc3ccccc23)cc1 ; c1ccc(-c2cccc3ccc(-c4ccc(N(c5ccc(-c6cccc7c6oc6ccccc67)cc5)c5cccc6ccccc56)cc4)cc23)cc1]

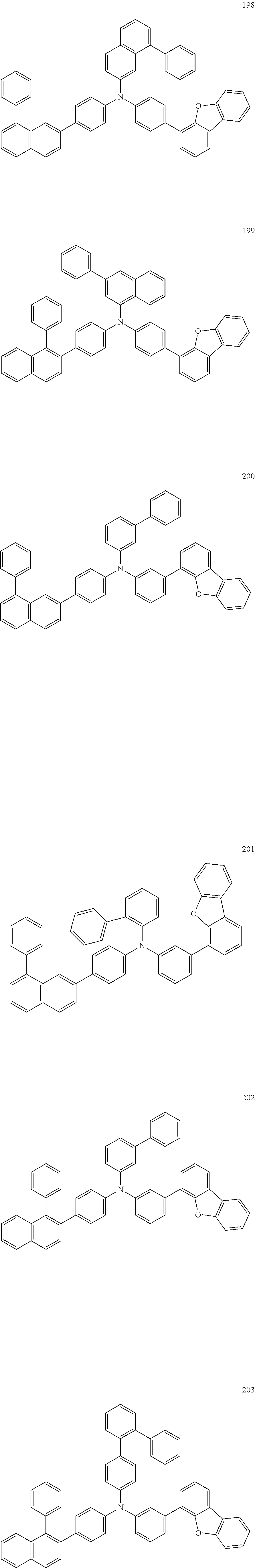

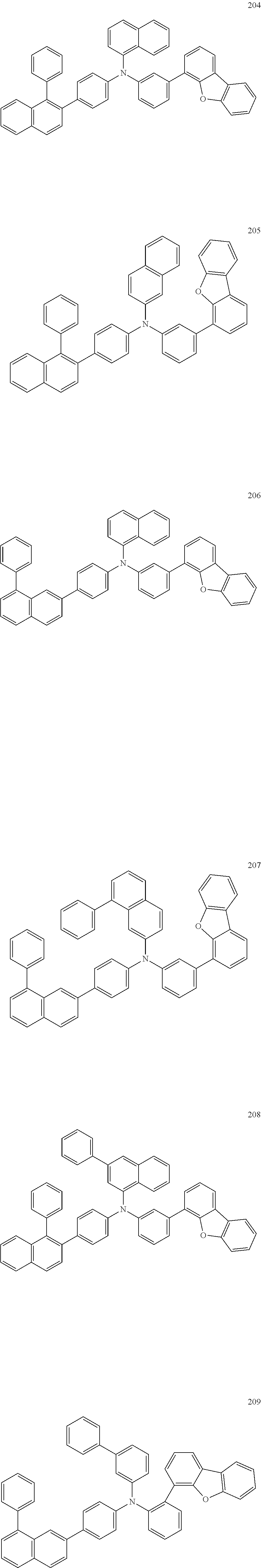

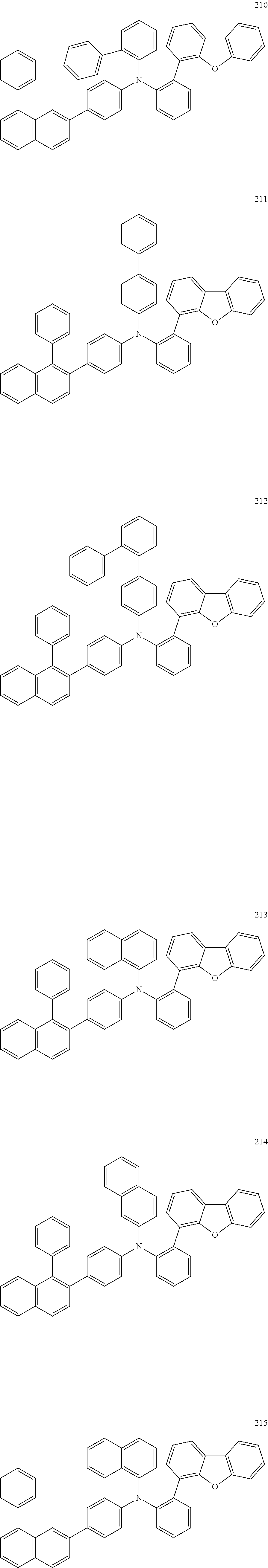

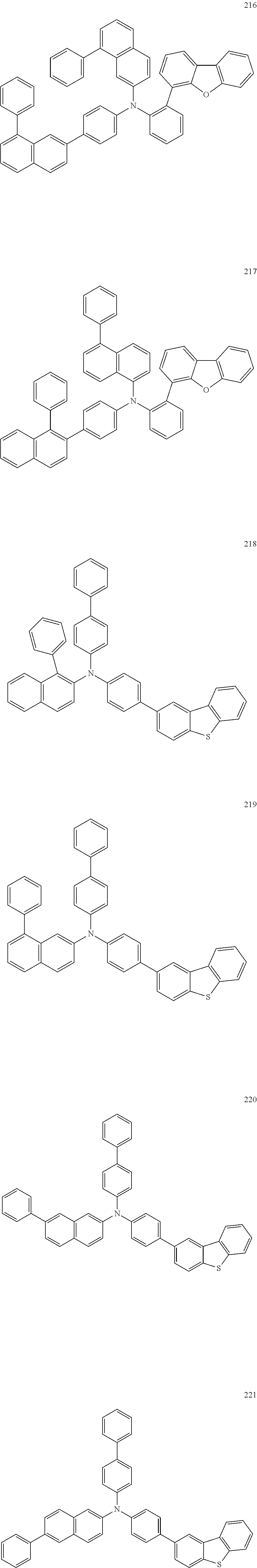

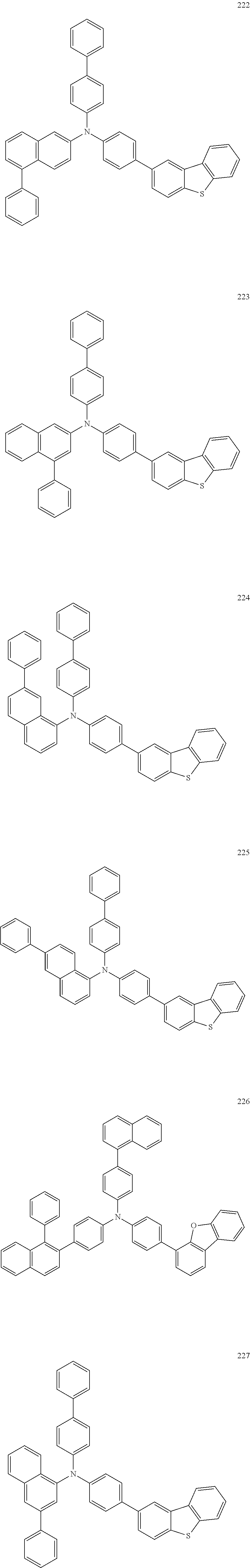

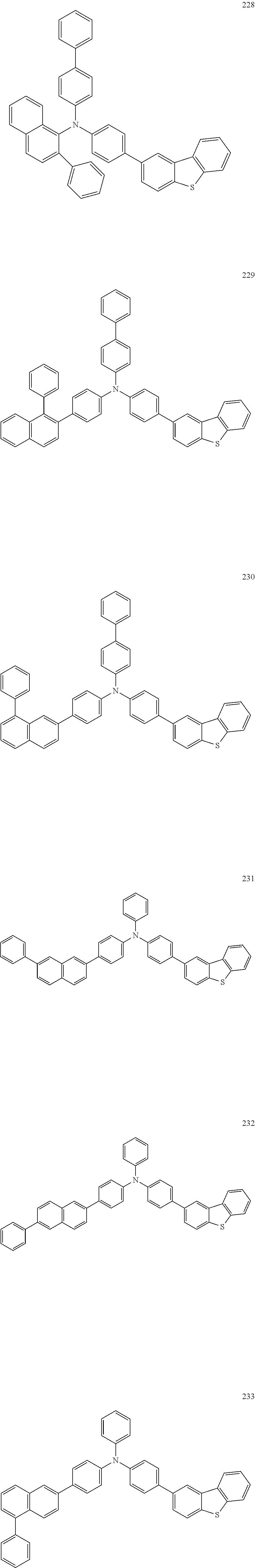

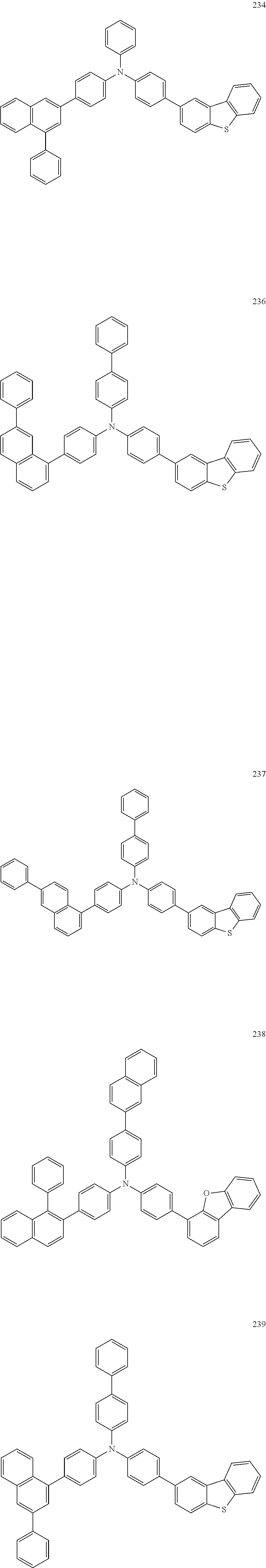 [SMILES: c1ccc(-c2cc(-c3ccc(N(c4ccccc4)c4ccc(-c5ccc6sc7ccccc7c6c5)cc4)cc3)cc3ccccc23)cc1 ; c1ccc(-c2ccc(N(c3ccc(-c4ccc5sc6ccccc6c5c4)cc3)c3ccc(-c4cccc5ccc(-c6ccccc6)cc45)cc3)cc2)cc1 ; c1ccc(-c2ccc(N(c3ccc(-c4ccc5sc6ccccc6c5c4)cc3)c3ccc(-c4cccc5cc(-c6ccccc6)ccc45)cc3)cc2)cc1 ; c1ccc(-c2c(-c3ccc(N(c4ccc(-c5ccc6ccccc6c5)cc4)c4ccc(-c5cccc6c5oc5ccccc56)cc4)cc3)ccc3ccccc23)cc1 ; c1ccc(-c2ccc(N(c3ccc(-c4ccc5sc6ccccc6c5c4)cc3)c3ccc(-c4cc(-c5ccccc5)cc5ccccc45)cc3)cc2)cc1]

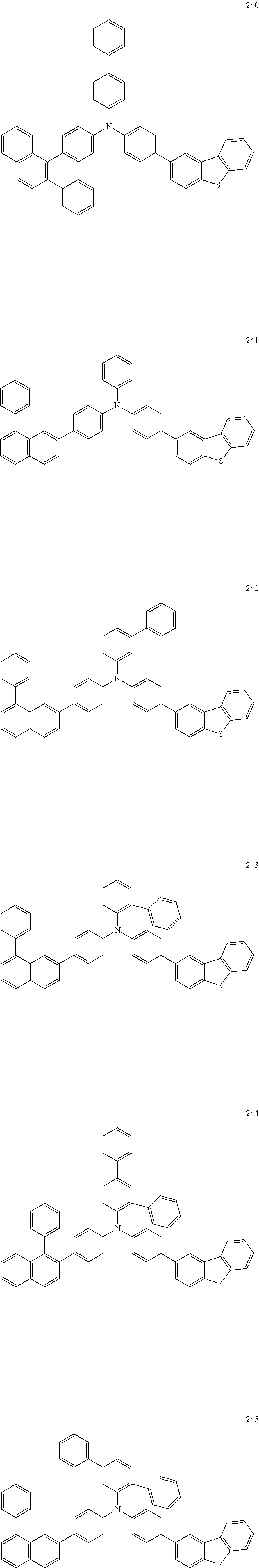

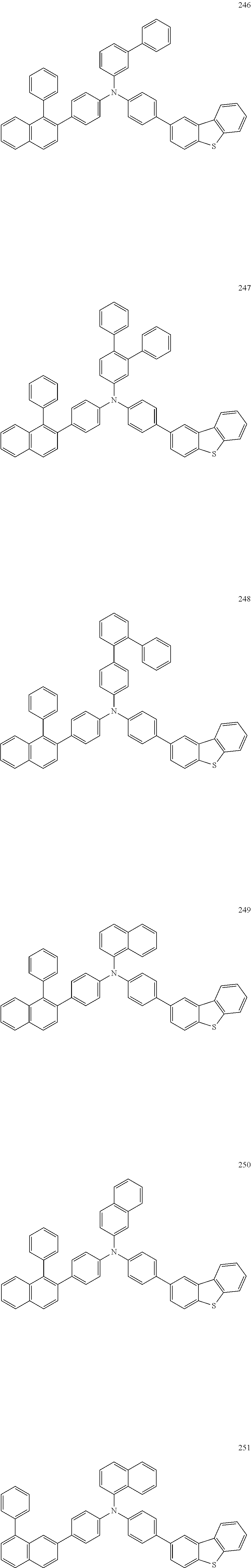

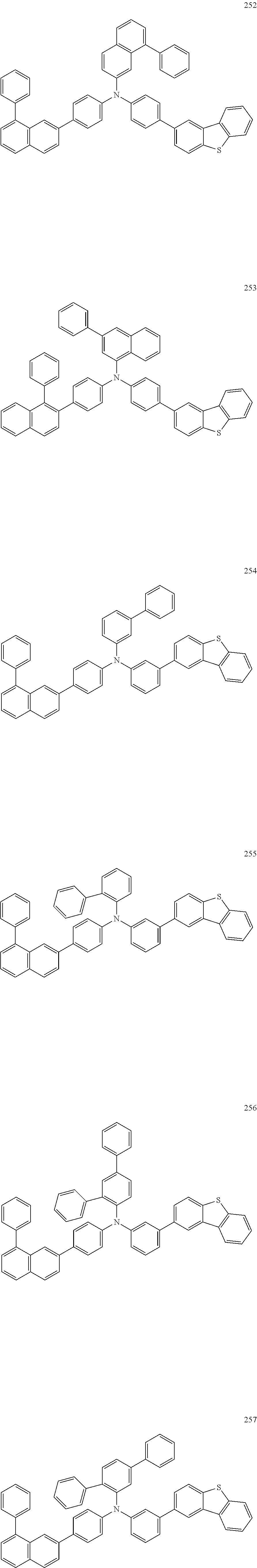

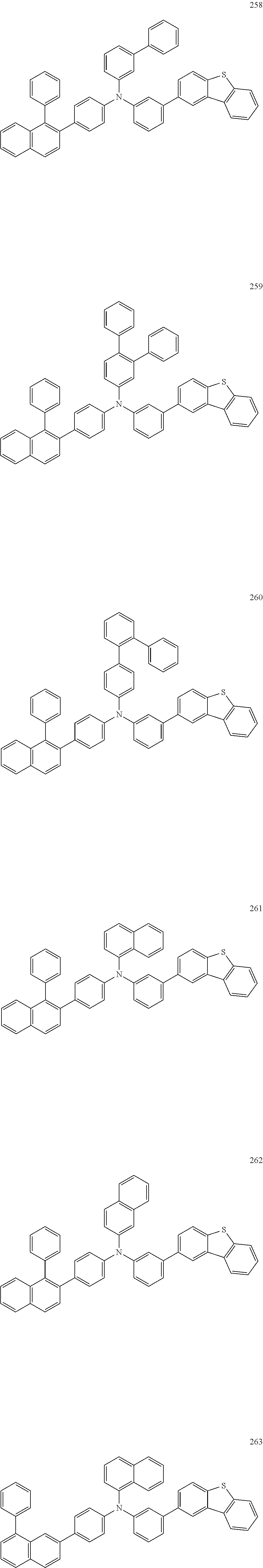

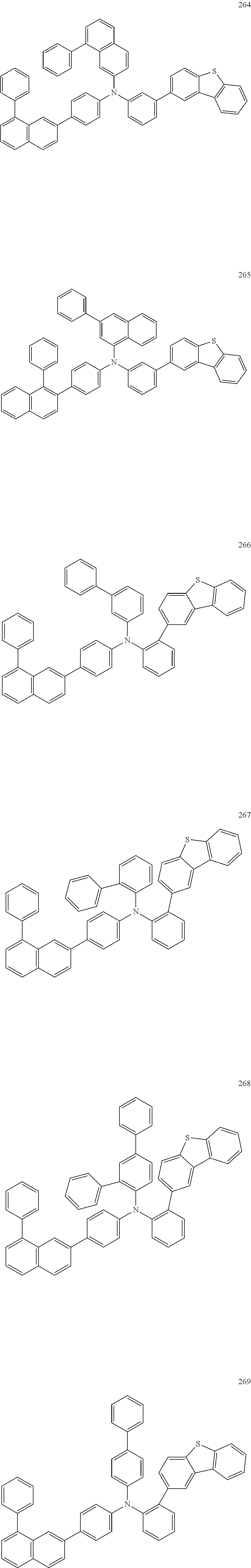

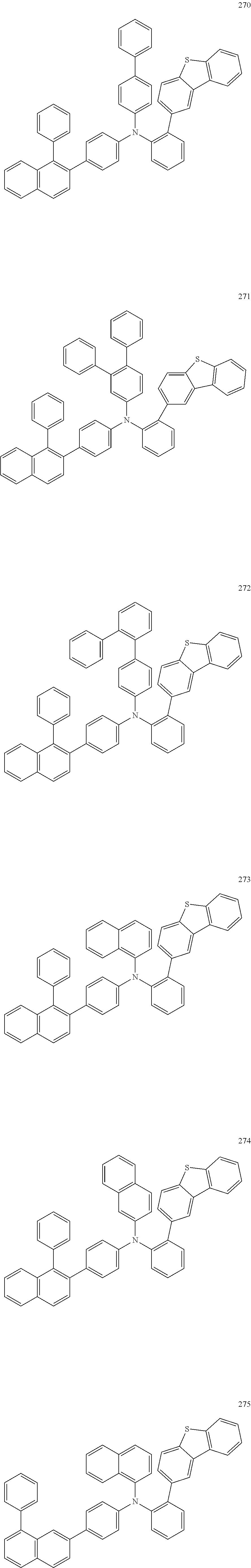

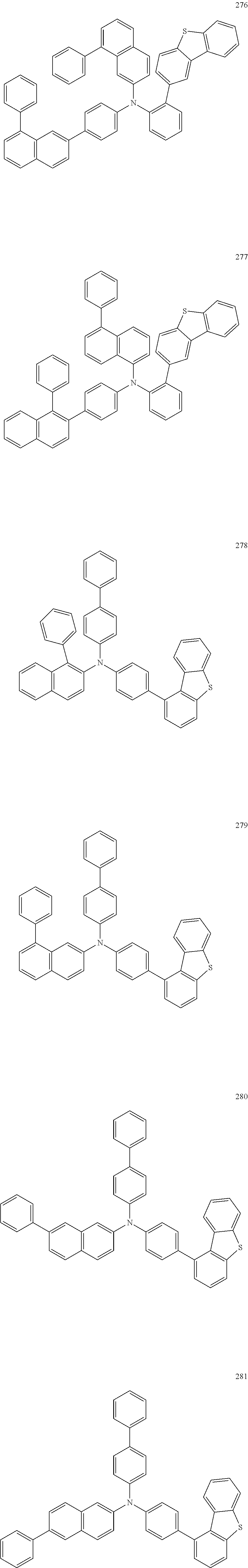

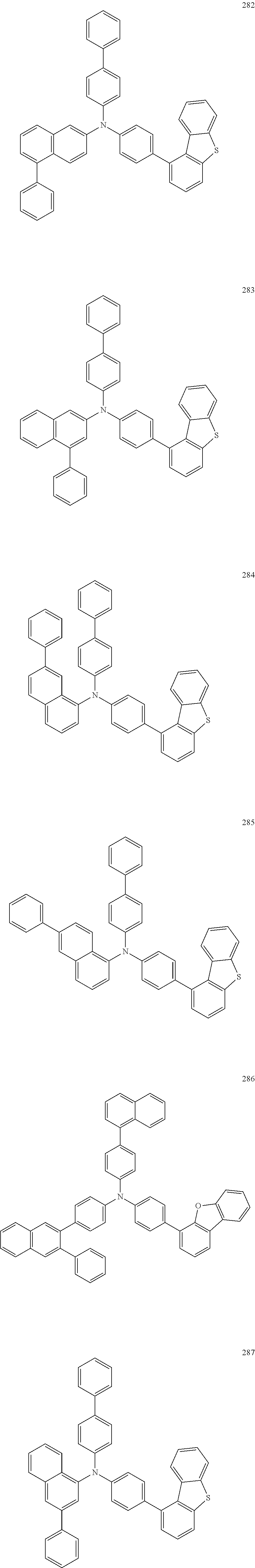

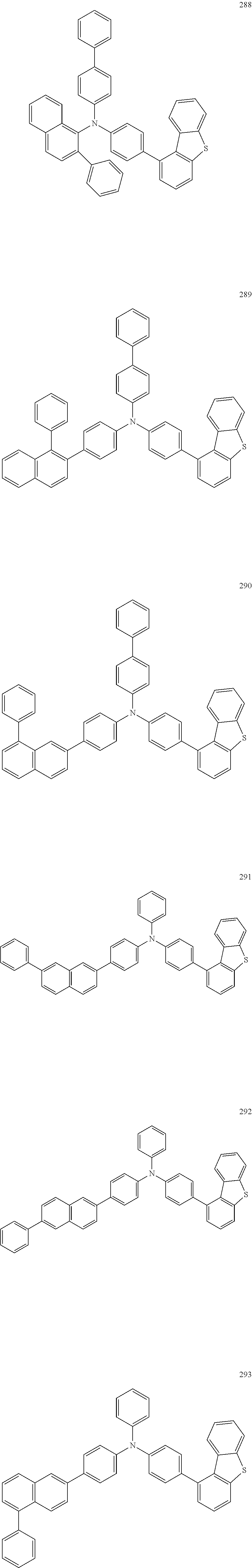

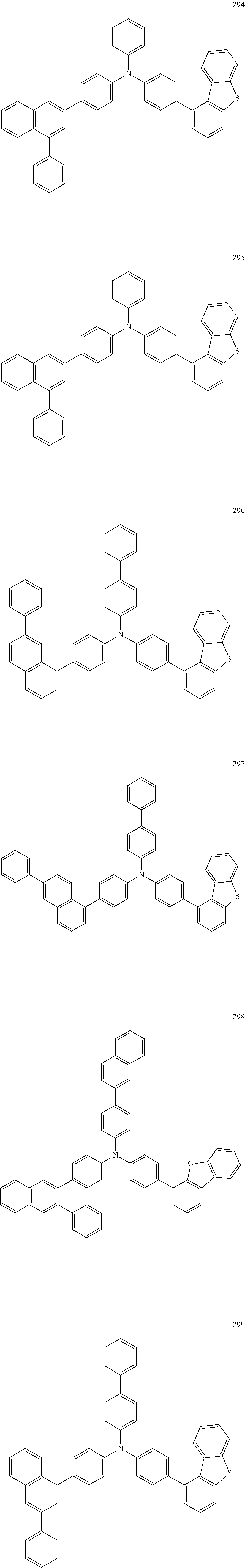

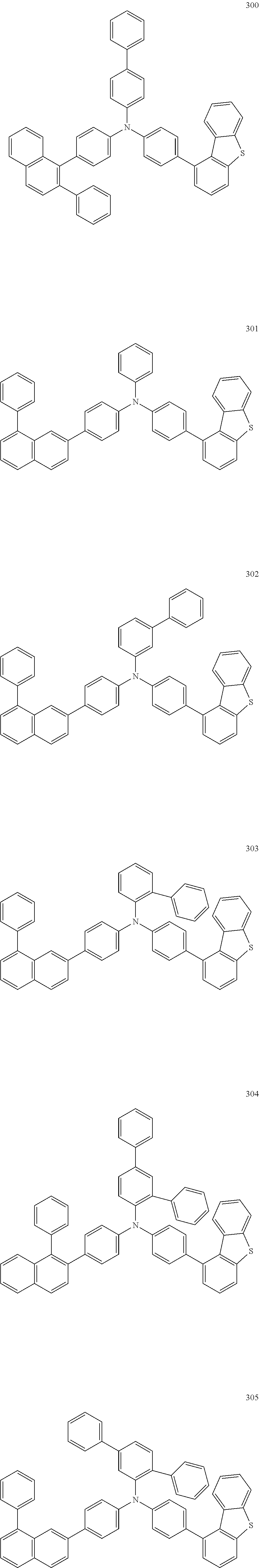

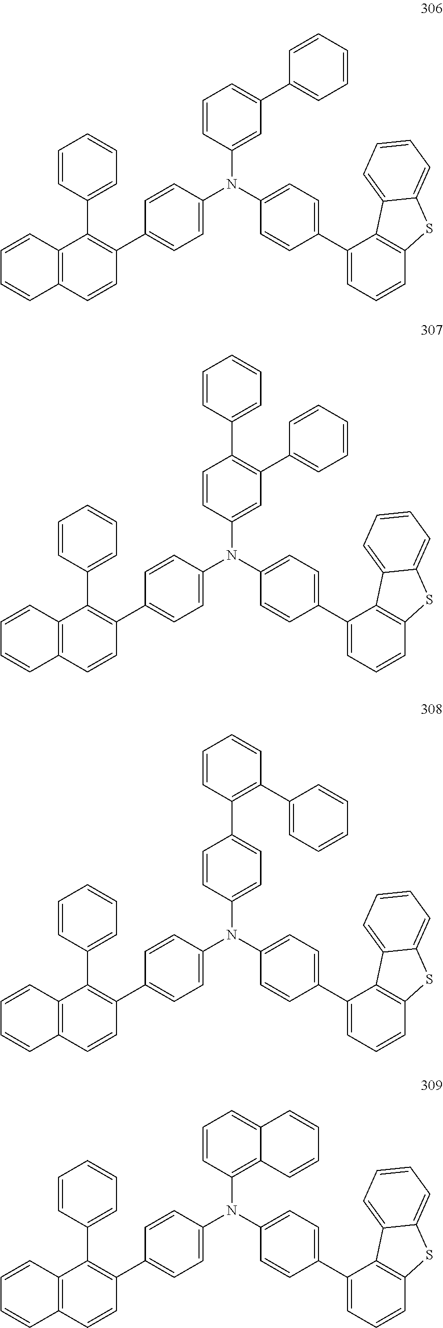 [SMILES: c1ccc(-c2cccc(N(c3ccc(-c4ccc5ccccc5c4-c4ccccc4)cc3)c3ccc(-c4cccc5sc6ccccc6c45)cc3)c2)cc1 ; c1ccc(-c2ccc(N(c3ccc(-c4ccc5ccccc5c4-c4ccccc4)cc3)c3ccc(-c4cccc5sc6ccccc6c45)cc3)cc2-c2ccccc2)cc1 ; c1ccc(-c2ccccc2-c2ccc(N(c3ccc(-c4ccc5ccccc5c4-c4ccccc4)cc3)c3ccc(-c4cccc5sc6ccccc6c45)cc3)cc2)cc1 ; c1ccc(-c2c(-c3ccc(N(c4ccc(-c5cccc6sc7ccccc7c56)cc4)c4cccc5ccccc45)cc3)ccc3ccccc23)cc1]

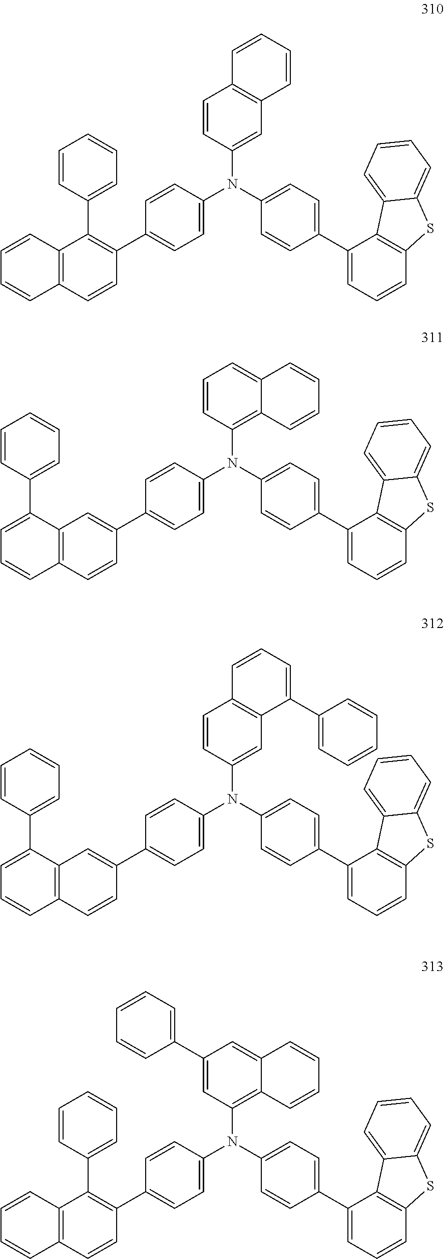 [SMILES: c1ccc(-c2c(-c3ccc(N(c4ccc(-c5cccc6sc7ccccc7c56)cc4)c4ccc5ccccc5c4)cc3)ccc3ccccc23)cc1 ; c1ccc(-c2cccc3ccc(-c4ccc(N(c5ccc(-c6cccc7sc8ccccc8c67)cc5)c5cccc6ccccc56)cc4)cc23)cc1 ; c1ccc(-c2cccc3ccc(-c4ccc(N(c5ccc(-c6cccc7sc8ccccc8c67)cc5)c5ccc6cccc(-c7ccccc7)c6c5)cc4)cc23)cc1 ; c1ccc(-c2cc(N(c3ccc(-c4ccc5ccccc5c4-c4ccccc4)cc3)c3ccc(-c4cccc5sc6ccccc6c45)cc3)c3ccccc3c2)cc1]

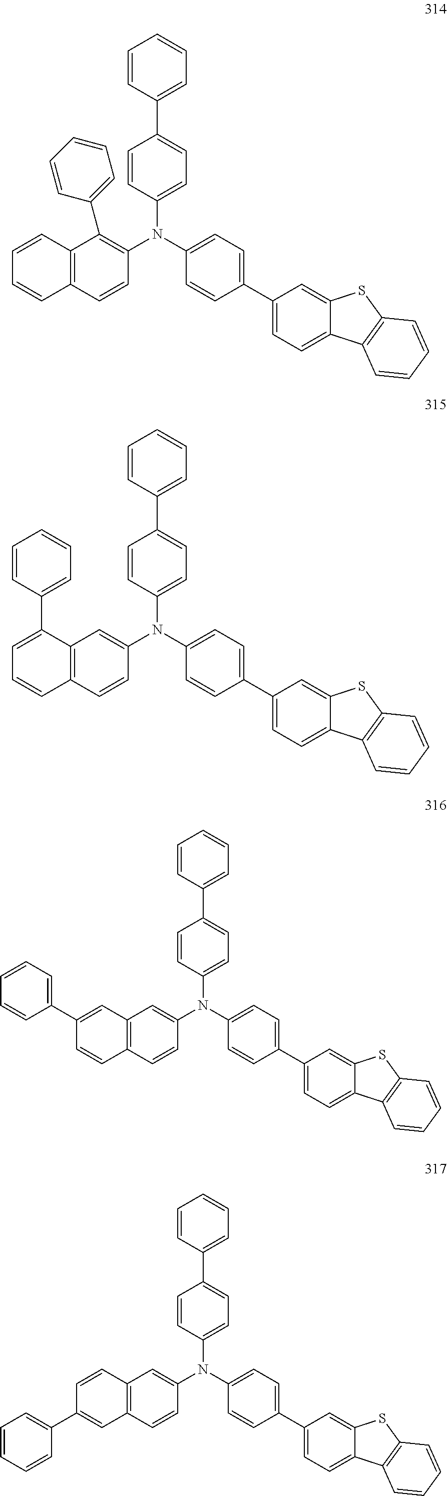 [SMILES: c1ccc(-c2ccc(N(c3ccc(-c4ccc5c(c4)sc4ccccc45)cc3)c3ccc4ccccc4c3-c3ccccc3)cc2)cc1 ; c1ccc(-c2ccc(N(c3ccc(-c4ccc5c(c4)sc4ccccc45)cc3)c3ccc4cccc(-c5ccccc5)c4c3)cc2)cc1 ; c1ccc(-c2ccc(N(c3ccc(-c4ccc5c(c4)sc4ccccc45)cc3)c3ccc4ccc(-c5ccccc5)cc4c3)cc2)cc1 ; c1ccc(-c2ccc(N(c3ccc(-c4ccc5c(c4)sc4ccccc45)cc3)c3ccc4cc(-c5ccccc5)ccc4c3)cc2)cc1]

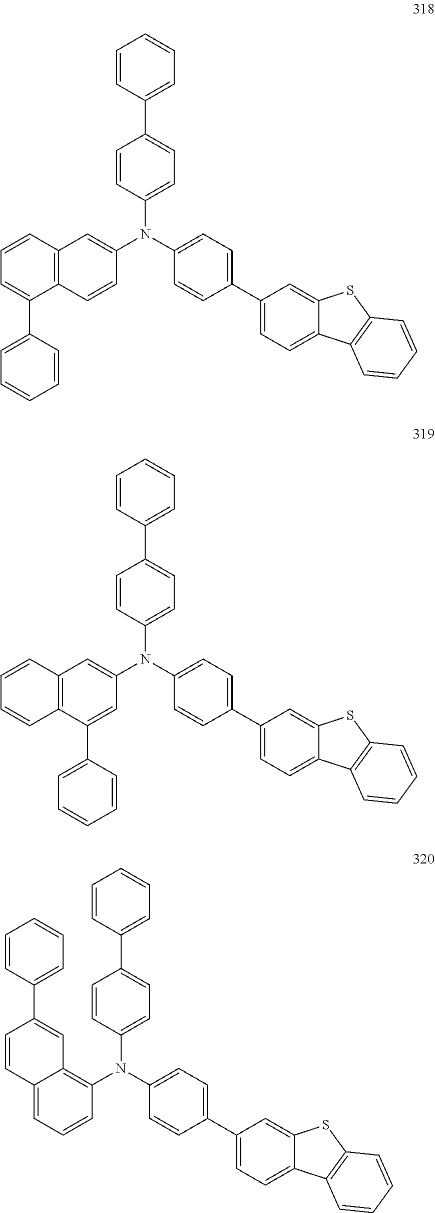 [SMILES: c1ccc(-c2ccc(N(c3ccc(-c4ccc5c(c4)sc4ccccc45)cc3)c3ccc4c(-c5ccccc5)cccc4c3)cc2)cc1 ; c1ccc(-c2ccc(N(c3ccc(-c4ccc5c(c4)sc4ccccc45)cc3)c3cc(-c4ccccc4)c4ccccc4c3)cc2)cc1 ; c1ccc(-c2ccc(N(c3ccc(-c4ccc5c(c4)sc4ccccc45)cc3)c3cccc4ccc(-c5ccccc5)cc34)cc2)cc1]

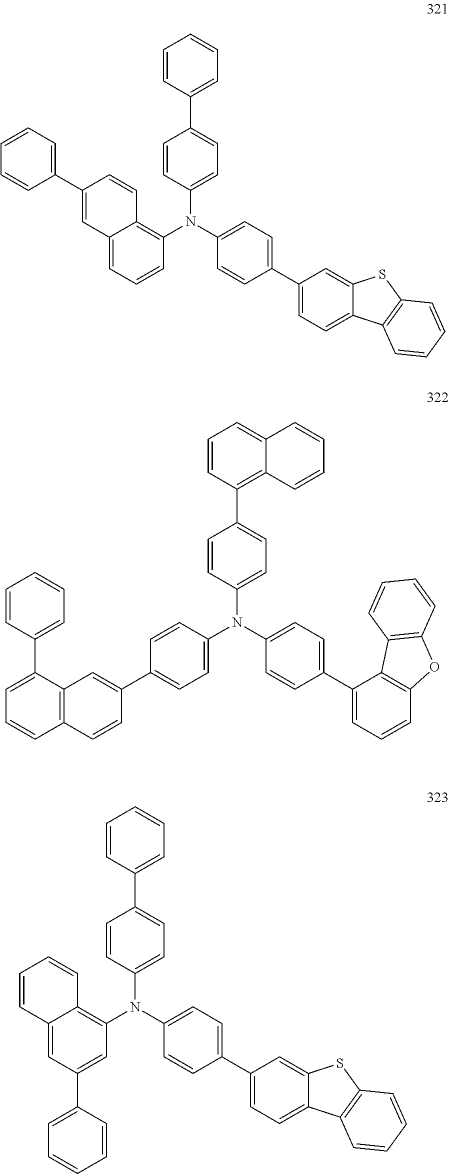 [SMILES: c1ccc(-c2ccc(N(c3ccc(-c4ccc5c(c4)sc4ccccc45)cc3)c3cccc4cc(-c5ccccc5)ccc34)cc2)cc1 ; c1ccc(-c2cccc3ccc(-c4ccc(N(c5ccc(-c6cccc7ccccc67)cc5)c5ccc(-c6cccc7oc8ccccc8c67)cc5)cc4)cc23)cc1 ; c1ccc(-c2ccc(N(c3ccc(-c4ccc5c(c4)sc4ccccc45)cc3)c3cc(-c4ccccc4)cc4ccccc34)cc2)cc1]

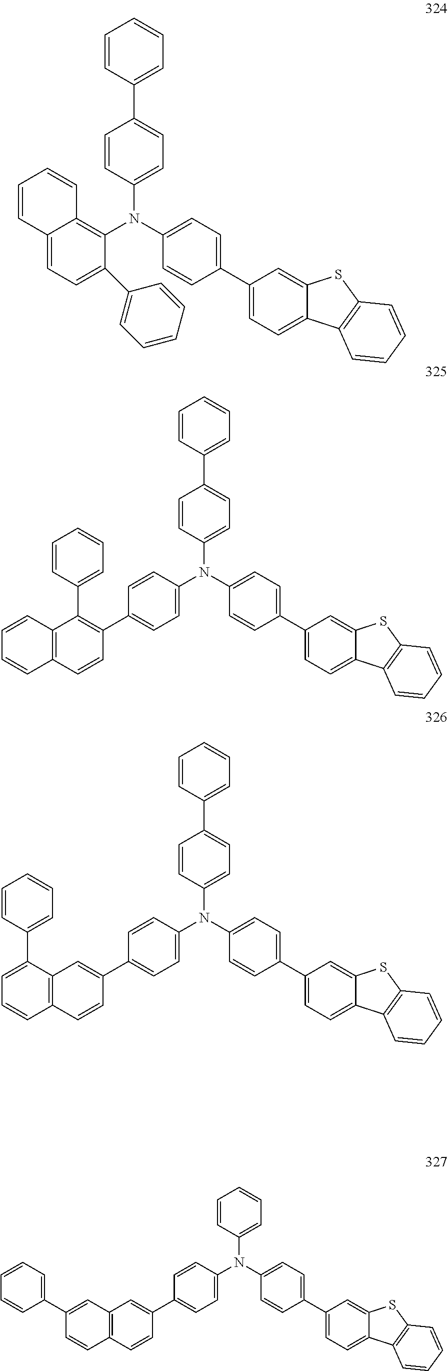 [SMILES: c1ccc(-c2ccc(N(c3ccc(-c4ccc5c(c4)sc4ccccc45)cc3)c3c(-c4ccccc4)ccc4ccccc34)cc2)cc1 ; c1ccc(-c2ccc(N(c3ccc(-c4ccc5c(c4)sc4ccccc45)cc3)c3ccc(-c4ccc5ccccc5c4-c4ccccc4)cc3)cc2)cc1 ; c1ccc(-c2ccc(N(c3ccc(-c4ccc5c(c4)sc4ccccc45)cc3)c3ccc(-c4ccc5cccc(-c6ccccc6)c5c4)cc3)cc2)cc1 ; c1ccc(-c2ccc3ccc(-c4ccc(N(c5ccccc5)c5ccc(-c6ccc7c(c6)sc6ccccc67)cc5)cc4)cc3c2)cc1]

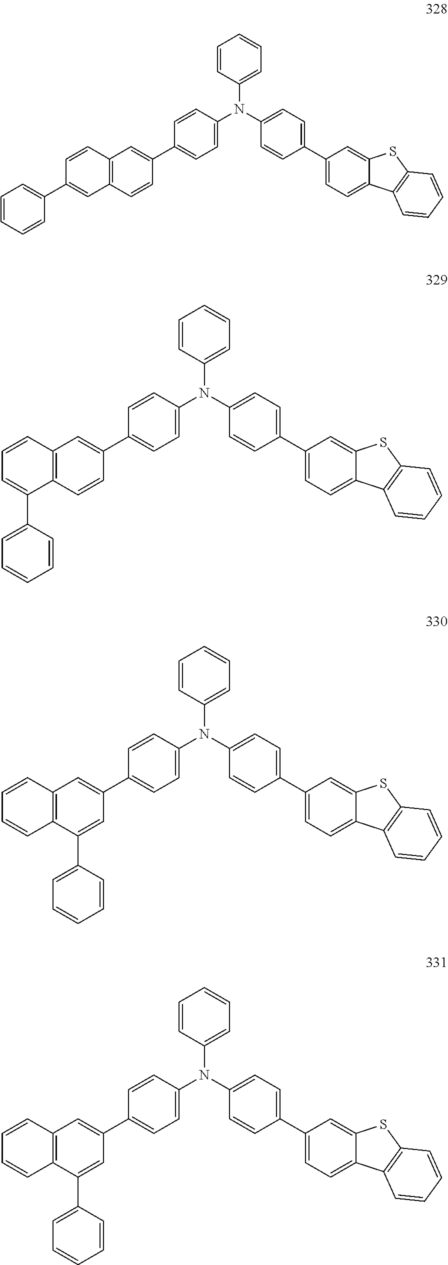 [SMILES: c1ccc(-c2ccc3cc(-c4ccc(N(c5ccccc5)c5ccc(-c6ccc7c(c6)sc6ccccc67)cc5)cc4)ccc3c2)cc1 ; c1ccc(-c2cccc3cc(-c4ccc(N(c5ccccc5)c5ccc(-c6ccc7c(c6)sc6ccccc67)cc5)cc4)ccc23)cc1 ; c1ccc(-c2cc(-c3ccc(N(c4ccccc4)c4ccc(-c5ccc6c(c5)sc5ccccc56)cc4)cc3)cc3ccccc23)cc1 ; c1ccc(-c2cc(-c3ccc(N(c4ccccc4)c4ccc(-c5ccc6c(c5)sc5ccccc56)cc4)cc3)cc3ccccc23)cc1]

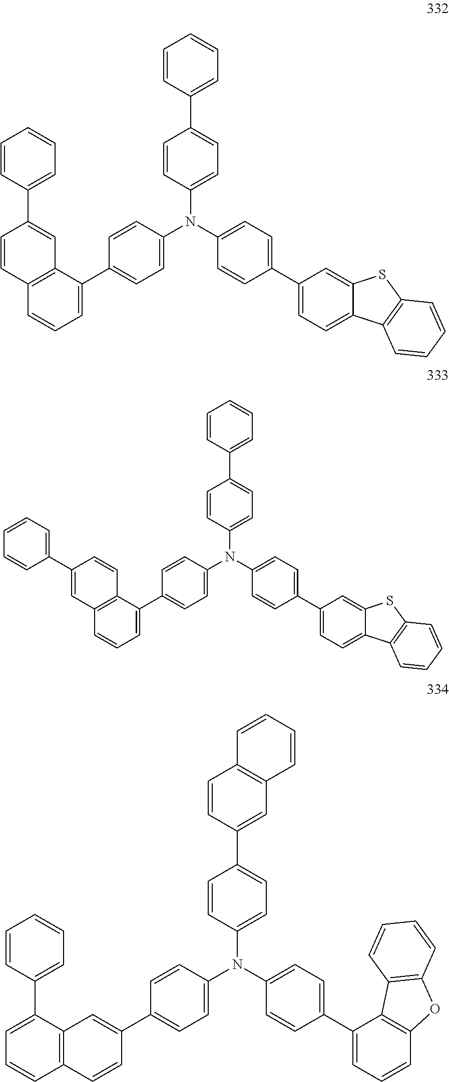 [SMILES: c1ccc(-c2ccc(N(c3ccc(-c4ccc5c(c4)sc4ccccc45)cc3)c3ccc(-c4cccc5ccc(-c6ccccc6)cc45)cc3)cc2)cc1 ; c1ccc(-c2ccc(N(c3ccc(-c4ccc5c(c4)sc4ccccc45)cc3)c3ccc(-c4cccc5cc(-c6ccccc6)ccc45)cc3)cc2)cc1 ; c1ccc(-c2cccc3ccc(-c4ccc(N(c5ccc(-c6ccc7ccccc7c6)cc5)c5ccc(-c6cccc7oc8ccccc8c67)cc5)cc4)cc23)cc1]

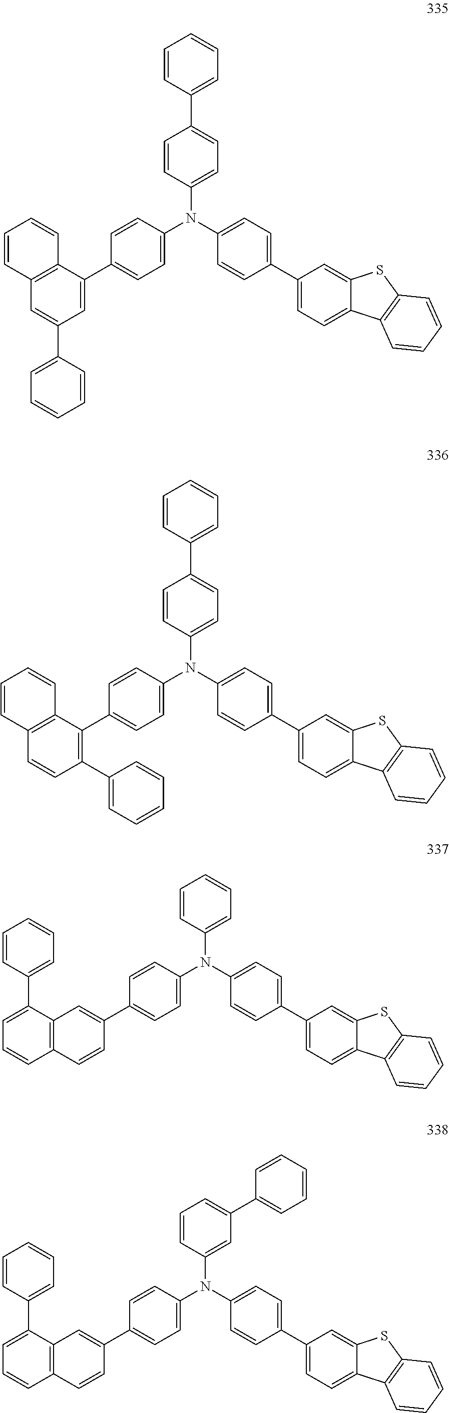 [SMILES: c1ccc(-c2ccc(N(c3ccc(-c4ccc5c(c4)sc4ccccc45)cc3)c3ccc(-c4cc(-c5ccccc5)cc5ccccc45)cc3)cc2)cc1 ; c1ccc(-c2ccc(N(c3ccc(-c4ccc5c(c4)sc4ccccc45)cc3)c3ccc(-c4c(-c5ccccc5)ccc5ccccc45)cc3)cc2)cc1 ; c1ccc(-c2cccc3ccc(-c4ccc(N(c5ccccc5)c5ccc(-c6ccc7c(c6)sc6ccccc67)cc5)cc4)cc23)cc1 ; c1ccc(-c2cccc(N(c3ccc(-c4ccc5c(c4)sc4ccccc45)cc3)c3ccc(-c4ccc5cccc(-c6ccccc6)c5c4)cc3)c2)cc1]

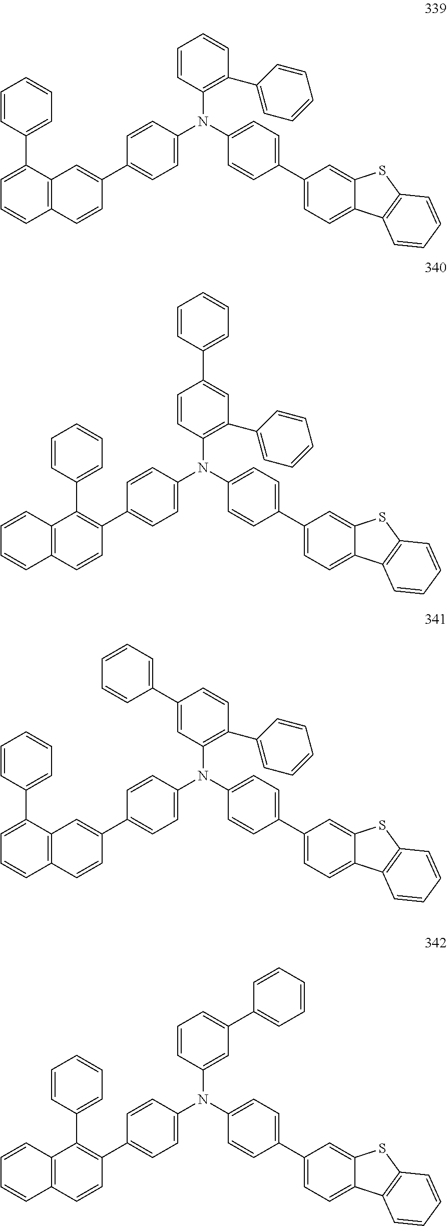 [SMILES: c1ccc(-c2ccccc2N(c2ccc(-c3ccc4c(c3)sc3ccccc34)cc2)c2ccc(-c3ccc4cccc(-c5ccccc5)c4c3)cc2)cc1 ; c1ccc(-c2ccc(N(c3ccc(-c4ccc5c(c4)sc4ccccc45)cc3)c3ccc(-c4ccc5ccccc5c4-c4ccccc4)cc3)c(-c3ccccc3)c2)cc1 ; c1ccc(-c2ccc(-c3ccccc3)c(N(c3ccc(-c4ccc5c(c4)sc4ccccc45)cc3)c3ccc(-c4ccc5cccc(-c6ccccc6)c5c4)cc3)c2)cc1 ; c1ccc(-c2cccc(N(c3ccc(-c4ccc5c(c4)sc4ccccc45)cc3)c3ccc(-c4ccc5ccccc5c4-c4ccccc4)cc3)c2)cc1]

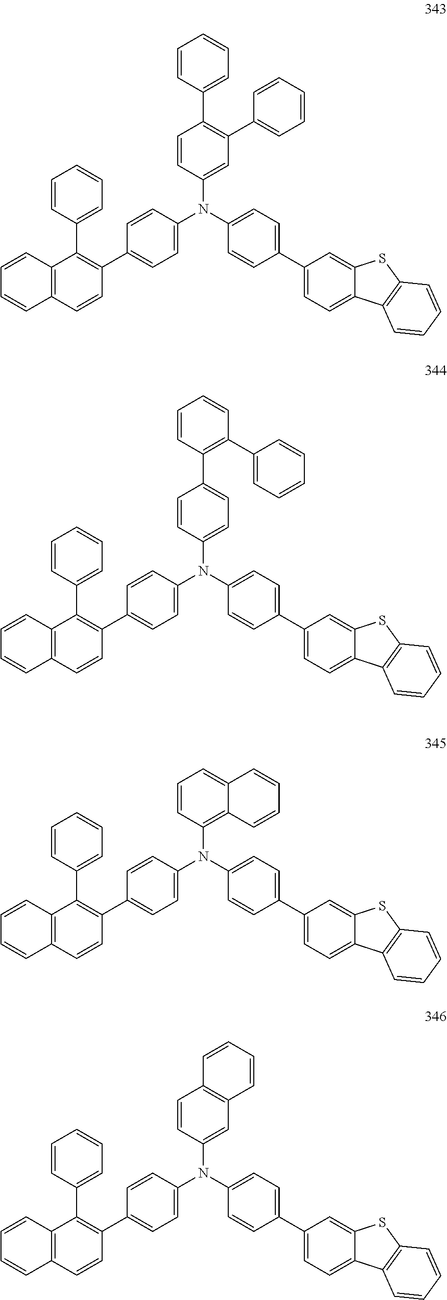 [SMILES: c1ccc(-c2ccc(N(c3ccc(-c4ccc5c(c4)sc4ccccc45)cc3)c3ccc(-c4ccc5ccccc5c4-c4ccccc4)cc3)cc2-c2ccccc2)cc1 ; c1ccc(-c2ccccc2-c2ccc(N(c3ccc(-c4ccc5c(c4)sc4ccccc45)cc3)c3ccc(-c4ccc5ccccc5c4-c4ccccc4)cc3)cc2)cc1 ; c1ccc(-c2c(-c3ccc(N(c4ccc(-c5ccc6c(c5)sc5ccccc56)cc4)c4cccc5ccccc45)cc3)ccc3ccccc23)cc1 ; c1ccc(-c2c(-c3ccc(N(c4ccc(-c5ccc6c(c5)sc5ccccc56)cc4)c4ccc5ccccc5c4)cc3)ccc3ccccc23)cc1]

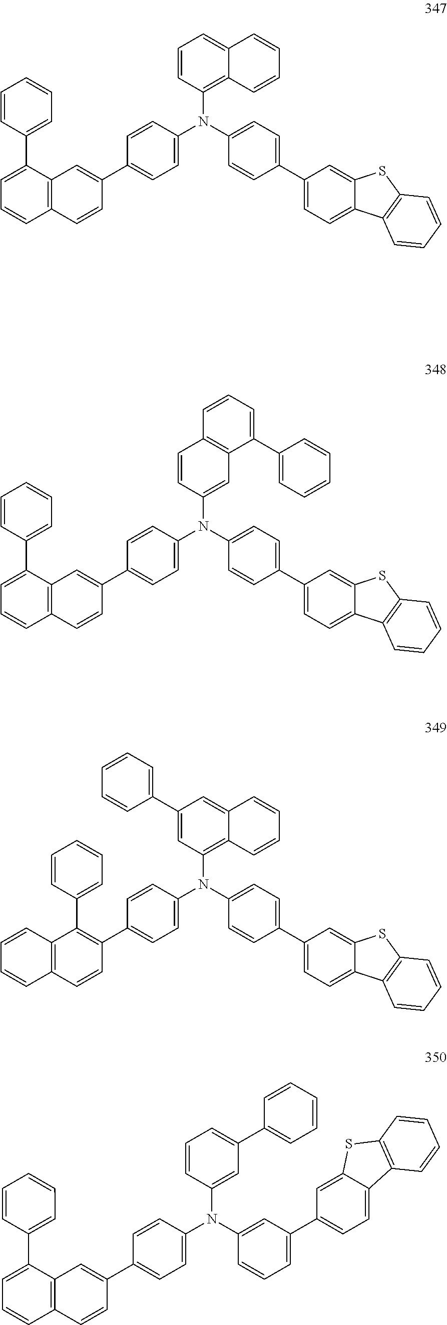 [SMILES: c1ccc(-c2cccc3ccc(-c4ccc(N(c5ccc(-c6ccc7c(c6)sc6ccccc67)cc5)c5cccc6ccccc56)cc4)cc23)cc1 ; c1ccc(-c2cccc3ccc(-c4ccc(N(c5ccc(-c6ccc7c(c6)sc6ccccc67)cc5)c5ccc6cccc(-c7ccccc7)c6c5)cc4)cc23)cc1 ; c1ccc(-c2cc(N(c3ccc(-c4ccc5c(c4)sc4ccccc45)cc3)c3ccc(-c4ccc5ccccc5c4-c4ccccc4)cc3)c3ccccc3c2)cc1 ; c1ccc(-c2cccc(N(c3ccc(-c4ccc5cccc(-c6ccccc6)c5c4)cc3)c3cccc(-c4ccc5c(c4)sc4ccccc45)c3)c2)cc1]

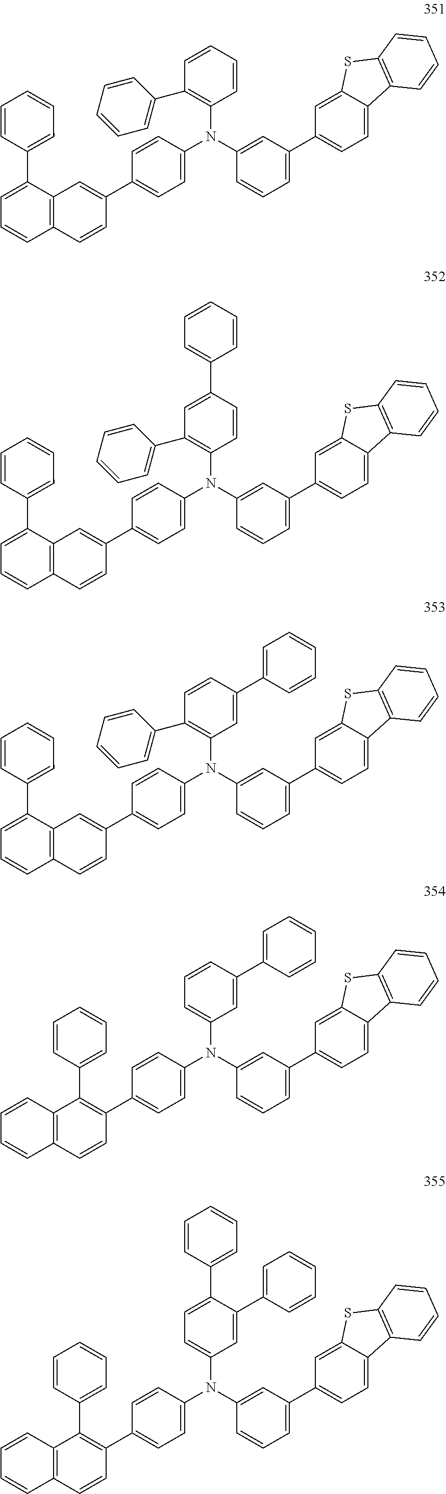 [SMILES: c1ccc(-c2ccccc2N(c2ccc(-c3ccc4cccc(-c5ccccc5)c4c3)cc2)c2cccc(-c3ccc4c(c3)sc3ccccc34)c2)cc1 ; c1ccc(-c2ccc(N(c3ccc(-c4ccc5cccc(-c6ccccc6)c5c4)cc3)c3cccc(-c4ccc5c(c4)sc4ccccc45)c3)c(-c3ccccc3)c2)cc1 ; c1ccc(-c2ccc(-c3ccccc3)c(N(c3ccc(-c4ccc5cccc(-c6ccccc6)c5c4)cc3)c3cccc(-c4ccc5c(c4)sc4ccccc45)c3)c2)cc1 ; c1ccc(-c2cccc(N(c3ccc(-c4ccc5ccccc5c4-c4ccccc4)cc3)c3cccc(-c4ccc5c(c4)sc4ccccc45)c3)c2)cc1 ; c1ccc(-c2ccc(N(c3ccc(-c4ccc5ccccc5c4-c4ccccc4)cc3)c3cccc(-c4ccc5c(c4)sc4ccccc45)c3)cc2-c2ccccc2)cc1]

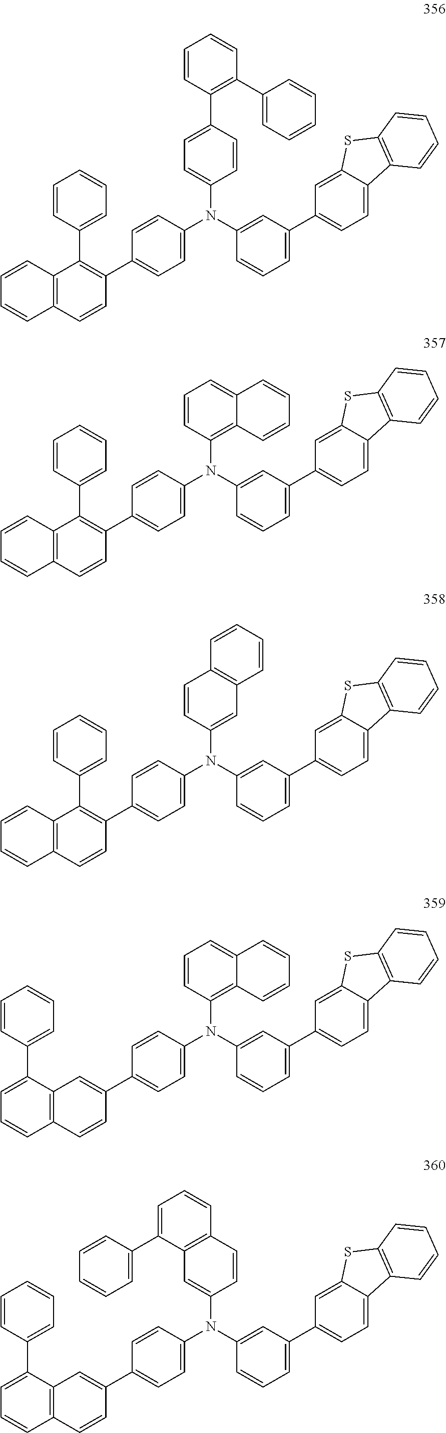 [SMILES: c1ccc(-c2ccccc2-c2ccc(N(c3ccc(-c4ccc5ccccc5c4-c4ccccc4)cc3)c3cccc(-c4ccc5c(c4)sc4ccccc45)c3)cc2)cc1 ; c1ccc(-c2c(-c3ccc(N(c4cccc(-c5ccc6c(c5)sc5ccccc56)c4)c4cccc5ccccc45)cc3)ccc3ccccc23)cc1 ; c1ccc(-c2c(-c3ccc(N(c4cccc(-c5ccc6c(c5)sc5ccccc56)c4)c4ccc5ccccc5c4)cc3)ccc3ccccc23)cc1 ; c1ccc(-c2cccc3ccc(-c4ccc(N(c5cccc(-c6ccc7c(c6)sc6ccccc67)c5)c5cccc6ccccc56)cc4)cc23)cc1 ; c1ccc(-c2cccc3ccc(-c4ccc(N(c5cccc(-c6ccc7c(c6)sc6ccccc67)c5)c5ccc6cccc(-c7ccccc7)c6c5)cc4)cc23)cc1]

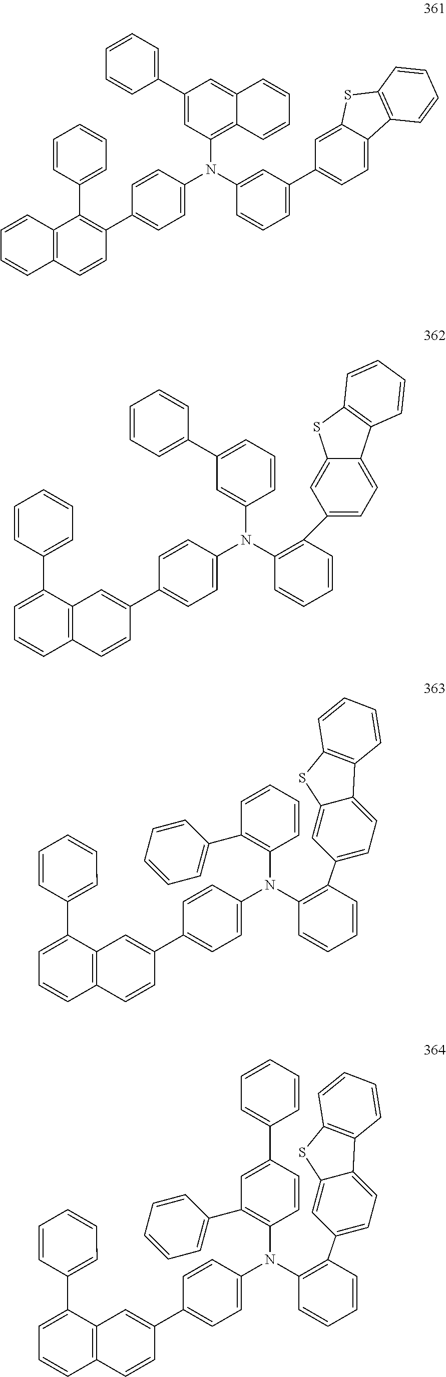 [SMILES: c1ccc(-c2cc(N(c3ccc(-c4ccc5ccccc5c4-c4ccccc4)cc3)c3cccc(-c4ccc5c(c4)sc4ccccc45)c3)c3ccccc3c2)cc1 ; c1ccc(-c2cccc(N(c3ccc(-c4ccc5cccc(-c6ccccc6)c5c4)cc3)c3ccccc3-c3ccc4c(c3)sc3ccccc34)c2)cc1 ; c1ccc(-c2ccccc2N(c2ccc(-c3ccc4cccc(-c5ccccc5)c4c3)cc2)c2ccccc2-c2ccc3c(c2)sc2ccccc23)cc1 ; c1ccc(-c2ccc(N(c3ccc(-c4ccc5cccc(-c6ccccc6)c5c4)cc3)c3ccccc3-c3ccc4c(c3)sc3ccccc34)c(-c3ccccc3)c2)cc1]

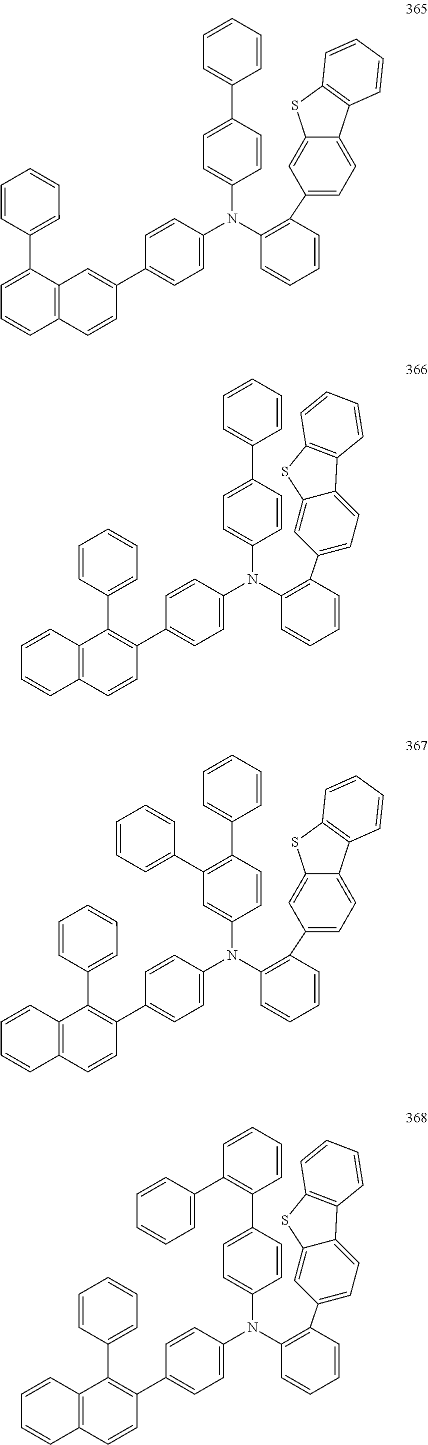 [SMILES: c1ccc(-c2ccc(N(c3ccc(-c4ccc5cccc(-c6ccccc6)c5c4)cc3)c3ccccc3-c3ccc4c(c3)sc3ccccc34)cc2)cc1 ; c1ccc(-c2ccc(N(c3ccc(-c4ccc5ccccc5c4-c4ccccc4)cc3)c3ccccc3-c3ccc4c(c3)sc3ccccc34)cc2)cc1 ; c1ccc(-c2ccc(N(c3ccc(-c4ccc5ccccc5c4-c4ccccc4)cc3)c3ccccc3-c3ccc4c(c3)sc3ccccc34)cc2-c2ccccc2)cc1 ; c1ccc(-c2ccccc2-c2ccc(N(c3ccc(-c4ccc5ccccc5c4-c4ccccc4)cc3)c3ccccc3-c3ccc4c(c3)sc3ccccc34)cc2)cc1]

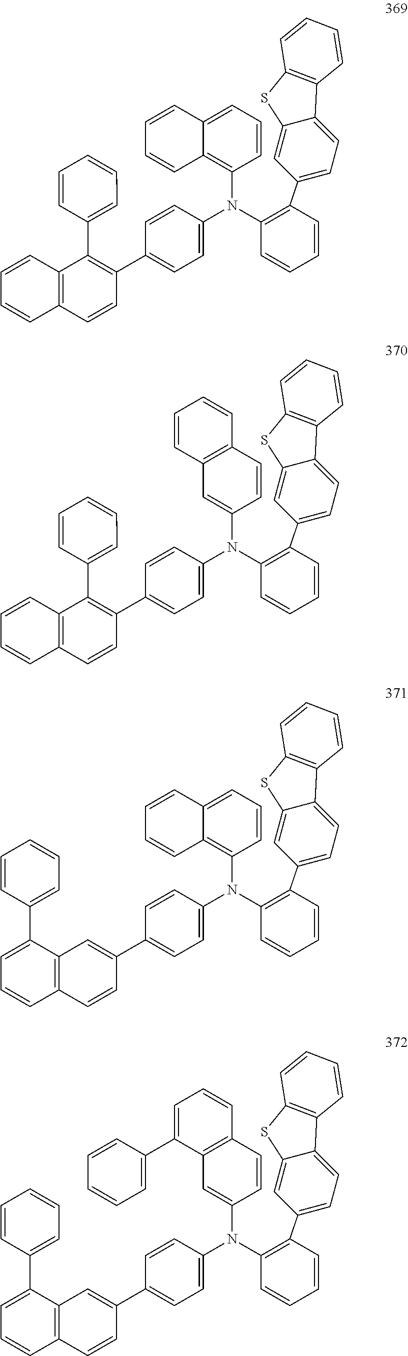 [SMILES: c1ccc(-c2c(-c3ccc(N(c4ccccc4-c4ccc5c(c4)sc4ccccc45)c4cccc5ccccc45)cc3)ccc3ccccc23)cc1 ; c1ccc(-c2c(-c3ccc(N(c4ccc5ccccc5c4)c4ccccc4-c4ccc5c(c4)sc4ccccc45)cc3)ccc3ccccc23)cc1 ; c1ccc(-c2cccc3ccc(-c4ccc(N(c5ccccc5-c5ccc6c(c5)sc5ccccc56)c5cccc6ccccc56)cc4)cc23)cc1 ; c1ccc(-c2cccc3ccc(-c4ccc(N(c5ccc6cccc(-c7ccccc7)c6c5)c5ccccc5-c5ccc6c(c5)sc5ccccc56)cc4)cc23)cc1]

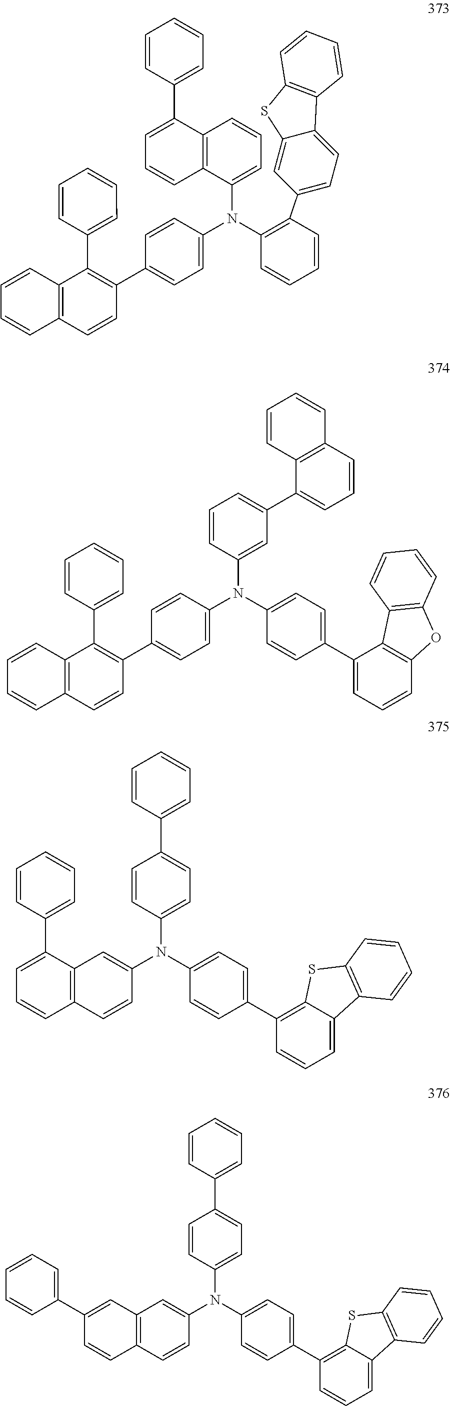 [SMILES: c1ccc(-c2c(-c3ccc(N(c4ccccc4-c4ccc5c(c4)sc4ccccc45)c4cccc5c(-c6ccccc6)cccc45)cc3)ccc3ccccc23)cc1 ; c1ccc(-c2c(-c3ccc(N(c4ccc(-c5cccc6oc7ccccc7c56)cc4)c4cccc(-c5cccc6ccccc56)c4)cc3)ccc3ccccc23)cc1 ; c1ccc(-c2ccc(N(c3ccc(-c4cccc5c4sc4ccccc45)cc3)c3ccc4cccc(-c5ccccc5)c4c3)cc2)cc1 ; c1ccc(-c2ccc(N(c3ccc(-c4cccc5c4sc4ccccc45)cc3)c3ccc4ccc(-c5ccccc5)cc4c3)cc2)cc1]

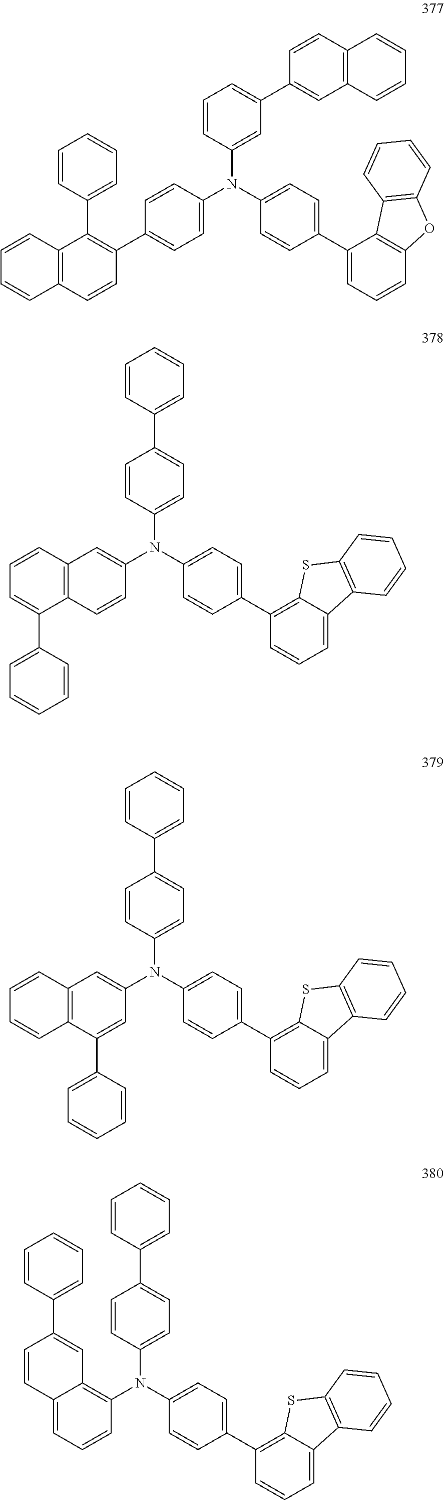 [SMILES: c1ccc(-c2c(-c3ccc(N(c4ccc(-c5cccc6oc7ccccc7c56)cc4)c4cccc(-c5ccc6ccccc6c5)c4)cc3)ccc3ccccc23)cc1 ; c1ccc(-c2ccc(N(c3ccc(-c4cccc5c4sc4ccccc45)cc3)c3ccc4c(-c5ccccc5)cccc4c3)cc2)cc1 ; c1ccc(-c2ccc(N(c3ccc(-c4cccc5c4sc4ccccc45)cc3)c3cc(-c4ccccc4)c4ccccc4c3)cc2)cc1 ; c1ccc(-c2ccc(N(c3ccc(-c4cccc5c4sc4ccccc45)cc3)c3cccc4ccc(-c5ccccc5)cc34)cc2)cc1]

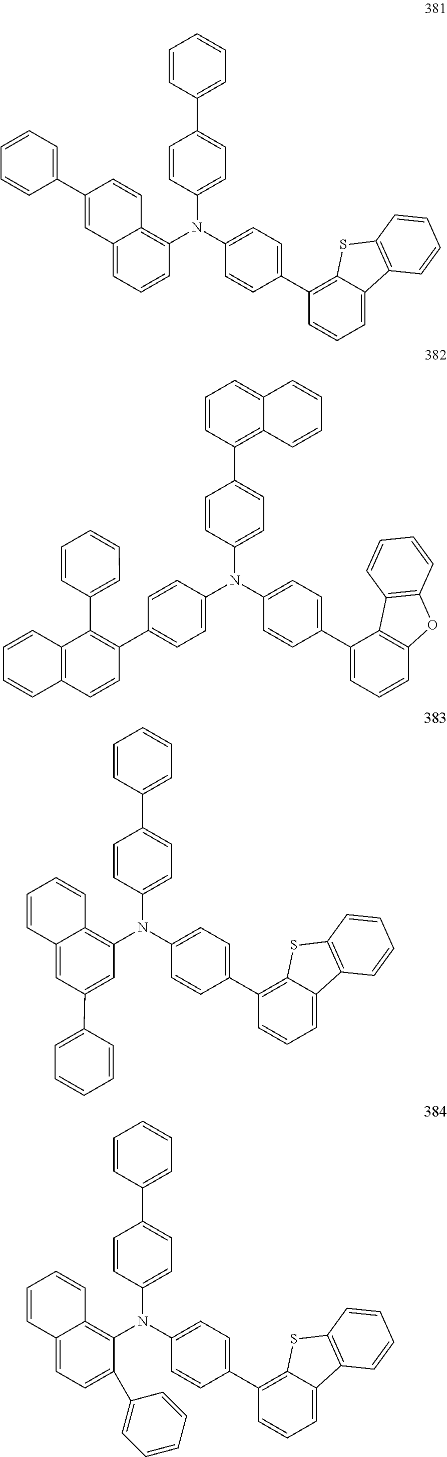 [SMILES: c1ccc(-c2ccc(N(c3ccc(-c4cccc5c4sc4ccccc45)cc3)c3cccc4cc(-c5ccccc5)ccc34)cc2)cc1 ; c1ccc(-c2c(-c3ccc(N(c4ccc(-c5cccc6ccccc56)cc4)c4ccc(-c5cccc6oc7ccccc7c56)cc4)cc3)ccc3ccccc23)cc1 ; c1ccc(-c2ccc(N(c3ccc(-c4cccc5c4sc4ccccc45)cc3)c3cc(-c4ccccc4)cc4ccccc34)cc2)cc1 ; c1ccc(-c2ccc(N(c3ccc(-c4cccc5c4sc4ccccc45)cc3)c3c(-c4ccccc4)ccc4ccccc34)cc2)cc1]

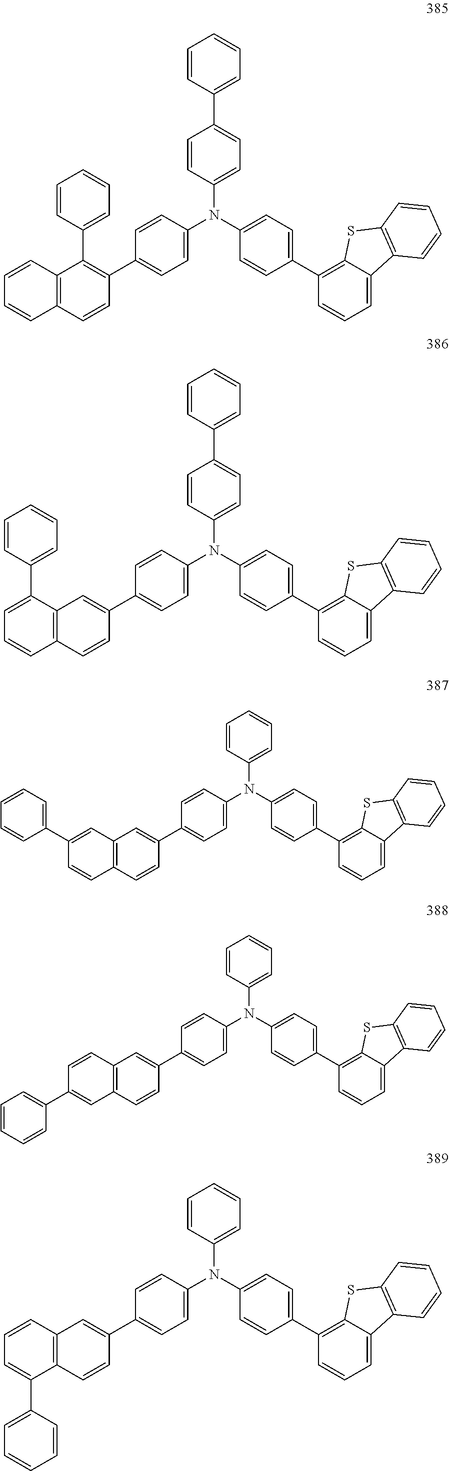 [SMILES: c1ccc(-c2ccc(N(c3ccc(-c4ccc5ccccc5c4-c4ccccc4)cc3)c3ccc(-c4cccc5c4sc4ccccc45)cc3)cc2)cc1 ; c1ccc(-c2ccc(N(c3ccc(-c4ccc5cccc(-c6ccccc6)c5c4)cc3)c3ccc(-c4cccc5c4sc4ccccc45)cc3)cc2)cc1 ; c1ccc(-c2ccc3ccc(-c4ccc(N(c5ccccc5)c5ccc(-c6cccc7c6sc6ccccc67)cc5)cc4)cc3c2)cc1 ; c1ccc(-c2ccc3cc(-c4ccc(N(c5ccccc5)c5ccc(-c6cccc7c6sc6ccccc67)cc5)cc4)ccc3c2)cc1 ; c1ccc(-c2cccc3cc(-c4ccc(N(c5ccccc5)c5ccc(-c6cccc7c6sc6ccccc67)cc5)cc4)ccc23)cc1]

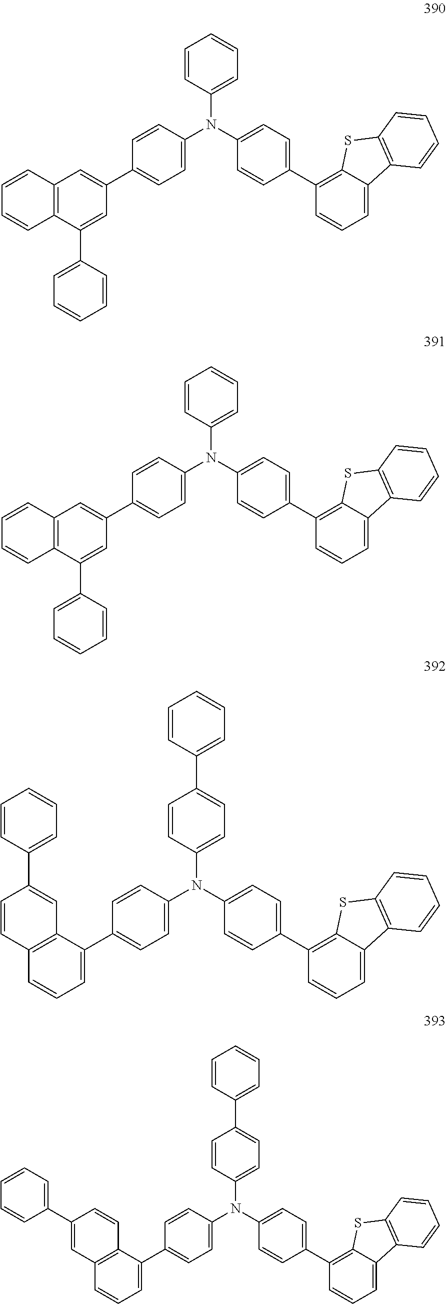 [SMILES: c1ccc(-c2cc(-c3ccc(N(c4ccccc4)c4ccc(-c5cccc6c5sc5ccccc56)cc4)cc3)cc3ccccc23)cc1 ; c1ccc(-c2cc(-c3ccc(N(c4ccccc4)c4ccc(-c5cccc6c5sc5ccccc56)cc4)cc3)cc3ccccc23)cc1 ; c1ccc(-c2ccc(N(c3ccc(-c4cccc5ccc(-c6ccccc6)cc45)cc3)c3ccc(-c4cccc5c4sc4ccccc45)cc3)cc2)cc1 ; c1ccc(-c2ccc(N(c3ccc(-c4cccc5cc(-c6ccccc6)ccc45)cc3)c3ccc(-c4cccc5c4sc4ccccc45)cc3)cc2)cc1]

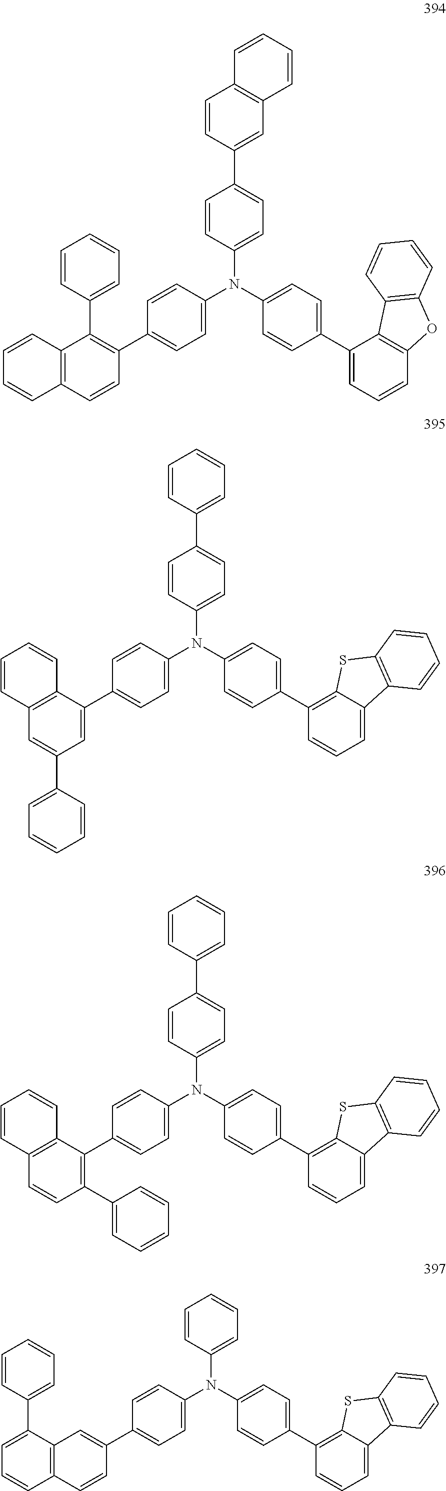 [SMILES: c1ccc(-c2c(-c3ccc(N(c4ccc(-c5ccc6ccccc6c5)cc4)c4ccc(-c5cccc6oc7ccccc7c56)cc4)cc3)ccc3ccccc23)cc1 ; c1ccc(-c2ccc(N(c3ccc(-c4cc(-c5ccccc5)cc5ccccc45)cc3)c3ccc(-c4cccc5c4sc4ccccc45)cc3)cc2)cc1 ; c1ccc(-c2ccc(N(c3ccc(-c4c(-c5ccccc5)ccc5ccccc45)cc3)c3ccc(-c4cccc5c4sc4ccccc45)cc3)cc2)cc1 ; c1ccc(-c2cccc3ccc(-c4ccc(N(c5ccccc5)c5ccc(-c6cccc7c6sc6ccccc67)cc5)cc4)cc23)cc1]

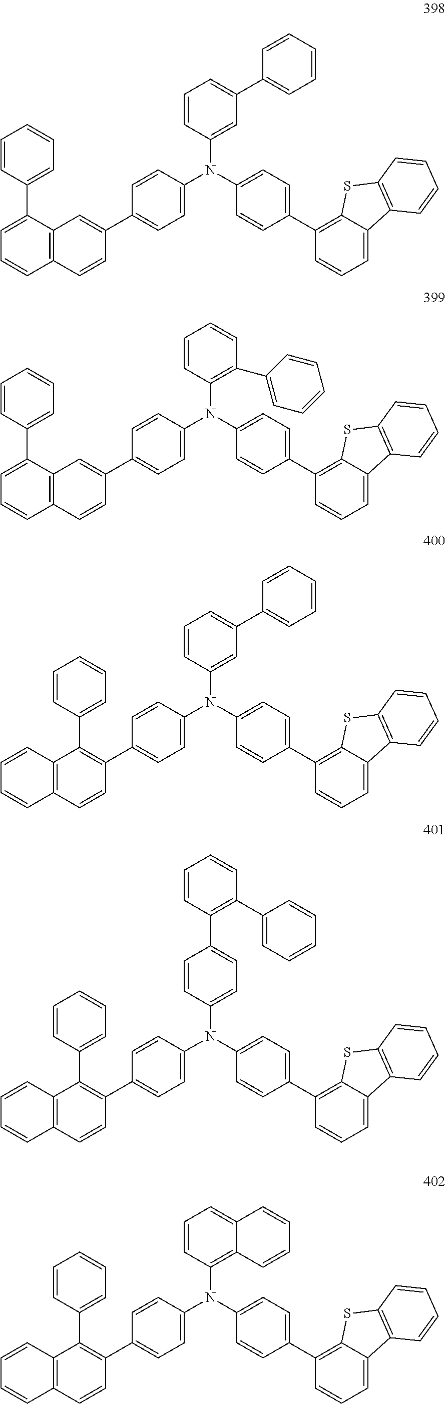 [SMILES: c1ccc(-c2cccc(N(c3ccc(-c4ccc5cccc(-c6ccccc6)c5c4)cc3)c3ccc(-c4cccc5c4sc4ccccc45)cc3)c2)cc1 ; c1ccc(-c2ccccc2N(c2ccc(-c3ccc4cccc(-c5ccccc5)c4c3)cc2)c2ccc(-c3cccc4c3sc3ccccc34)cc2)cc1 ; c1ccc(-c2cccc(N(c3ccc(-c4ccc5ccccc5c4-c4ccccc4)cc3)c3ccc(-c4cccc5c4sc4ccccc45)cc3)c2)cc1 ; c1ccc(-c2ccccc2-c2ccc(N(c3ccc(-c4ccc5ccccc5c4-c4ccccc4)cc3)c3ccc(-c4cccc5c4sc4ccccc45)cc3)cc2)cc1 ; c1ccc(-c2c(-c3ccc(N(c4ccc(-c5cccc6c5sc5ccccc56)cc4)c4cccc5ccccc45)cc3)ccc3ccccc23)cc1]

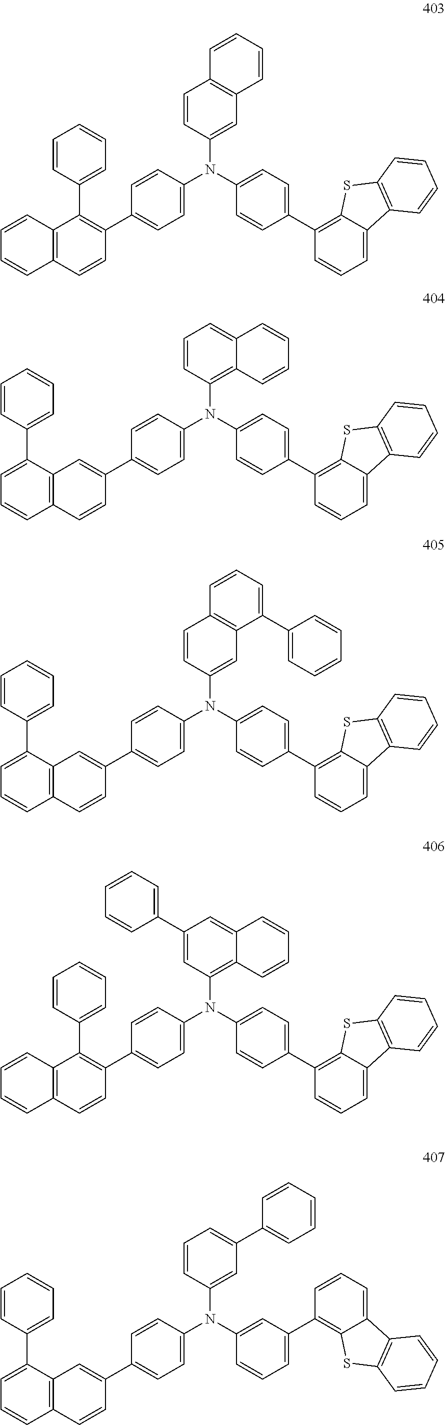 [SMILES: c1ccc(-c2c(-c3ccc(N(c4ccc(-c5cccc6c5sc5ccccc56)cc4)c4ccc5ccccc5c4)cc3)ccc3ccccc23)cc1 ; c1ccc(-c2cccc3ccc(-c4ccc(N(c5ccc(-c6cccc7c6sc6ccccc67)cc5)c5cccc6ccccc56)cc4)cc23)cc1 ; c1ccc(-c2cccc3ccc(-c4ccc(N(c5ccc(-c6cccc7c6sc6ccccc67)cc5)c5ccc6cccc(-c7ccccc7)c6c5)cc4)cc23)cc1 ; c1ccc(-c2cc(N(c3ccc(-c4ccc5ccccc5c4-c4ccccc4)cc3)c3ccc(-c4cccc5c4sc4ccccc45)cc3)c3ccccc3c2)cc1 ; c1ccc(-c2cccc(N(c3ccc(-c4ccc5cccc(-c6ccccc6)c5c4)cc3)c3cccc(-c4cccc5c4sc4ccccc45)c3)c2)cc1]

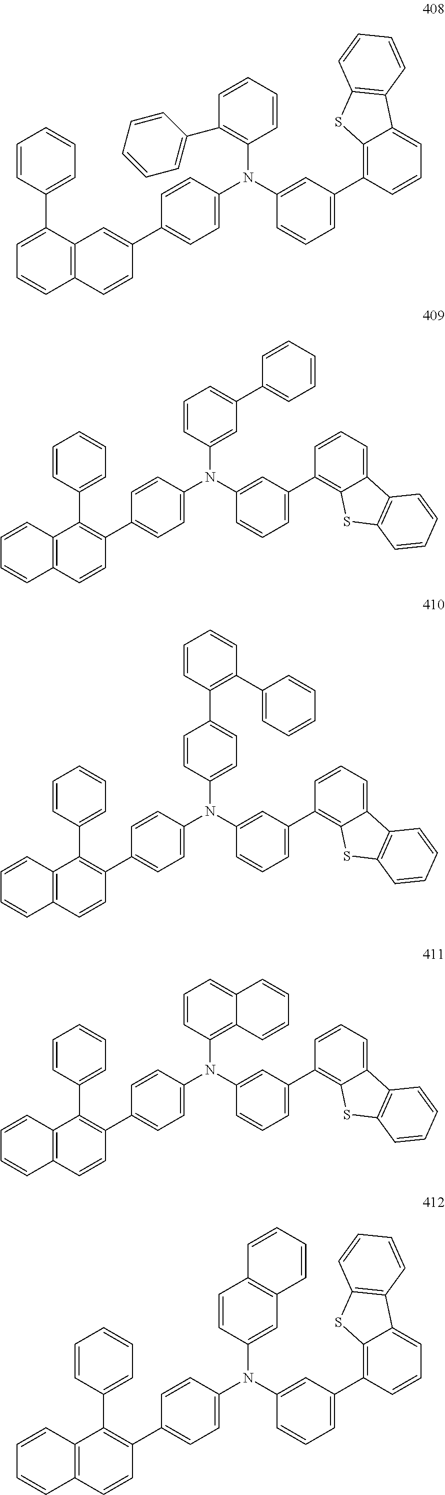 [SMILES: c1ccc(-c2ccccc2N(c2ccc(-c3ccc4cccc(-c5ccccc5)c4c3)cc2)c2cccc(-c3cccc4c3sc3ccccc34)c2)cc1 ; c1ccc(-c2cccc(N(c3ccc(-c4ccc5ccccc5c4-c4ccccc4)cc3)c3cccc(-c4cccc5c4sc4ccccc45)c3)c2)cc1 ; c1ccc(-c2ccccc2-c2ccc(N(c3ccc(-c4ccc5ccccc5c4-c4ccccc4)cc3)c3cccc(-c4cccc5c4sc4ccccc45)c3)cc2)cc1 ; c1ccc(-c2c(-c3ccc(N(c4cccc(-c5cccc6c5sc5ccccc56)c4)c4cccc5ccccc45)cc3)ccc3ccccc23)cc1 ; c1ccc(-c2c(-c3ccc(N(c4cccc(-c5cccc6c5sc5ccccc56)c4)c4ccc5ccccc5c4)cc3)ccc3ccccc23)cc1]

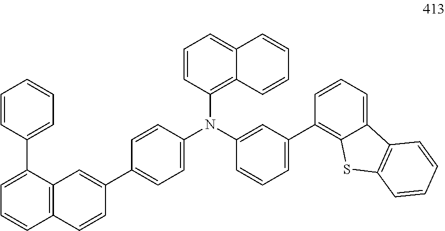 [SMILES: c1ccc(-c2cccc3ccc(-c4ccc(N(c5cccc(-c6cccc7c6sc6ccccc67)c5)c5cccc6ccccc56)cc4)cc23)cc1]

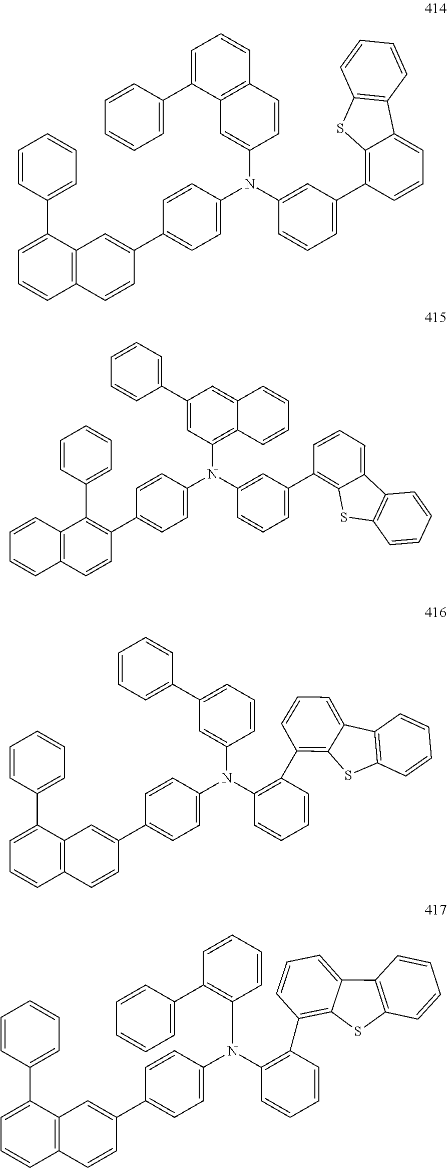 [SMILES: c1ccc(-c2cccc3ccc(-c4ccc(N(c5cccc(-c6cccc7c6sc6ccccc67)c5)c5ccc6cccc(-c7ccccc7)c6c5)cc4)cc23)cc1 ; c1ccc(-c2cc(N(c3ccc(-c4ccc5ccccc5c4-c4ccccc4)cc3)c3cccc(-c4cccc5c4sc4ccccc45)c3)c3ccccc3c2)cc1 ; c1ccc(-c2cccc(N(c3ccc(-c4ccc5cccc(-c6ccccc6)c5c4)cc3)c3ccccc3-c3cccc4c3sc3ccccc34)c2)cc1 ; c1ccc(-c2ccccc2N(c2ccc(-c3ccc4cccc(-c5ccccc5)c4c3)cc2)c2ccccc2-c2cccc3c2sc2ccccc23)cc1]

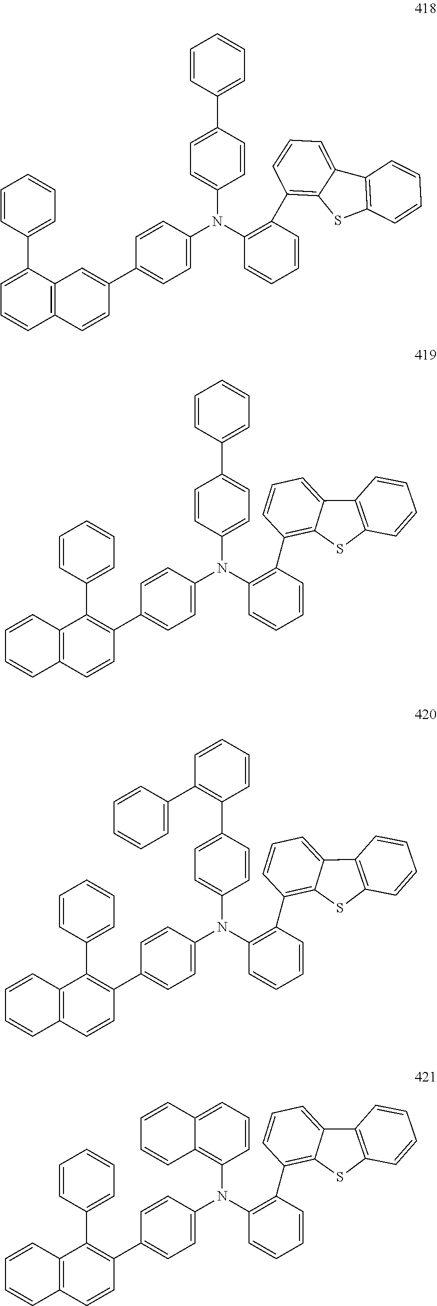 [SMILES: c1ccc(-c2ccc(N(c3ccc(-c4ccc5cccc(-c6ccccc6)c5c4)cc3)c3ccccc3-c3cccc4c3sc3ccccc34)cc2)cc1 ; c1ccc(-c2ccc(N(c3ccc(-c4ccc5ccccc5c4-c4ccccc4)cc3)c3ccccc3-c3cccc4c3sc3ccccc34)cc2)cc1 ; c1ccc(-c2ccccc2-c2ccc(N(c3ccc(-c4ccc5ccccc5c4-c4ccccc4)cc3)c3ccccc3-c3cccc4c3sc3ccccc34)cc2)cc1 ; c1ccc(-c2c(-c3ccc(N(c4ccccc4-c4cccc5c4sc4ccccc45)c4cccc5ccccc45)cc3)ccc3ccccc23)cc1]

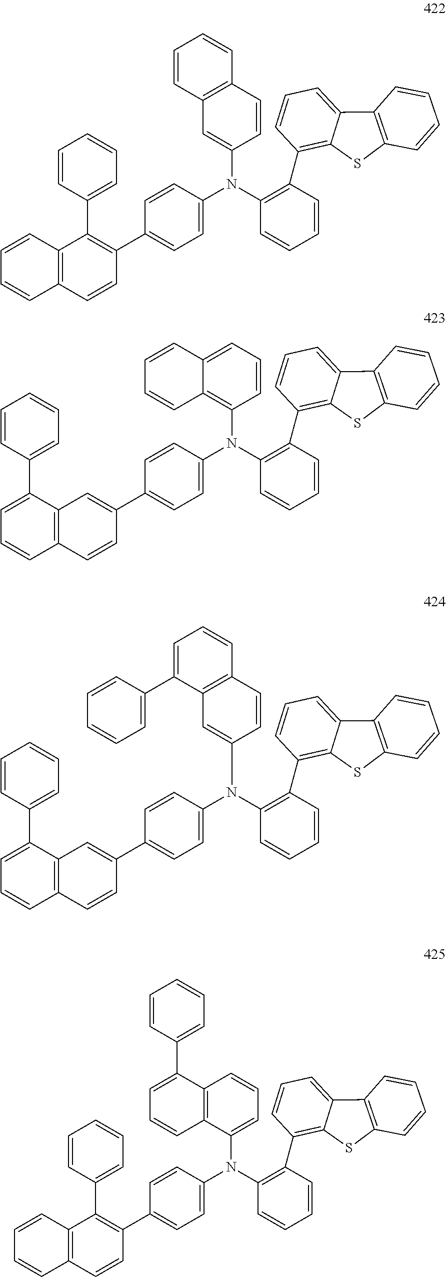 [SMILES: c1ccc(-c2c(-c3ccc(N(c4ccc5ccccc5c4)c4ccccc4-c4cccc5c4sc4ccccc45)cc3)ccc3ccccc23)cc1 ; c1ccc(-c2cccc3ccc(-c4ccc(N(c5ccccc5-c5cccc6c5sc5ccccc56)c5cccc6ccccc56)cc4)cc23)cc1 ; c1ccc(-c2cccc3ccc(-c4ccc(N(c5ccc6cccc(-c7ccccc7)c6c5)c5ccccc5-c5cccc6c5sc5ccccc56)cc4)cc23)cc1 ; c1ccc(-c2c(-c3ccc(N(c4ccccc4-c4cccc5c4sc4ccccc45)c4cccc5c(-c6ccccc6)cccc45)cc3)ccc3ccccc23)cc1]

In the light emitting element ED according to an aspect, the hole transport region HTR may further include a compound represented by Formula H-1 below. For example, the light emitting element ED according to an aspect may include a compound represented by Formula H-1 in another layer of the hole transport region HTR that does not include the amine compound according to an aspect in Formula 1 described above. However, aspects of the present disclosure are not limited thereto.

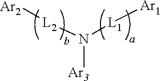

In Formula H-1 above, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer of 0 to 10. Meanwhile, when a or b is an integer of 2 or greater, a plurality of L₁'s and L₂'s may each independently be a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. In addition, in Formula H-1, Ar₃ may be a substituted or unsubstituted aryl group having 6 to 30 ring-forming carbon atoms, or a substituted or unsubstituted heteroaryl group having 2 to 30 ring-forming carbon atoms.

The compound represented by Formula H-1 above may be a monoamine compound. Alternatively, the compound represented by Formula H-1 may be a diamine compound in which at least one of Ar-₁ to Ar₃ includes an amine group as a substituent. In addition, the compound represented by Formula H-1 above may be a carbazole-based compound including a substituted or unsubstituted carbazole moiety in at least one of Ar₁ or Ar₂ or a substituted or unsubstituted fluorene-based group including a substituted or unsubstituted fluorene moiety in at least one of Ar₁ or Ar₂.

The compound represented by Formula H-1 may be represented by any one of compounds from Compound Group H below. However, the compounds listed in Compound Group H below are presented as an example, and the compound represented by Formula H-1 is not limited to the those listed in Compound Group H below.

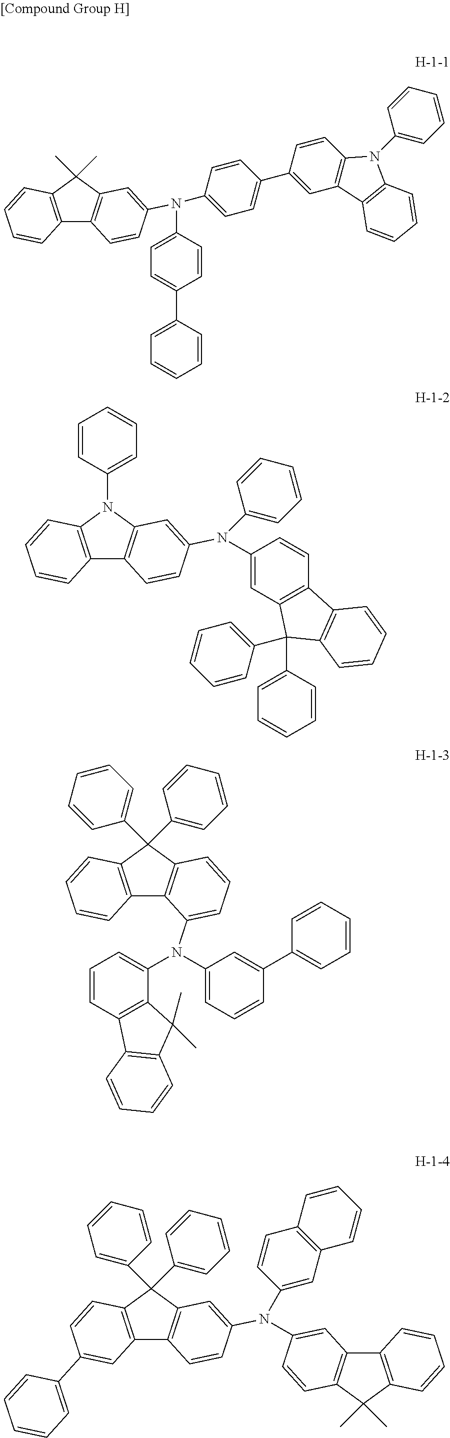 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

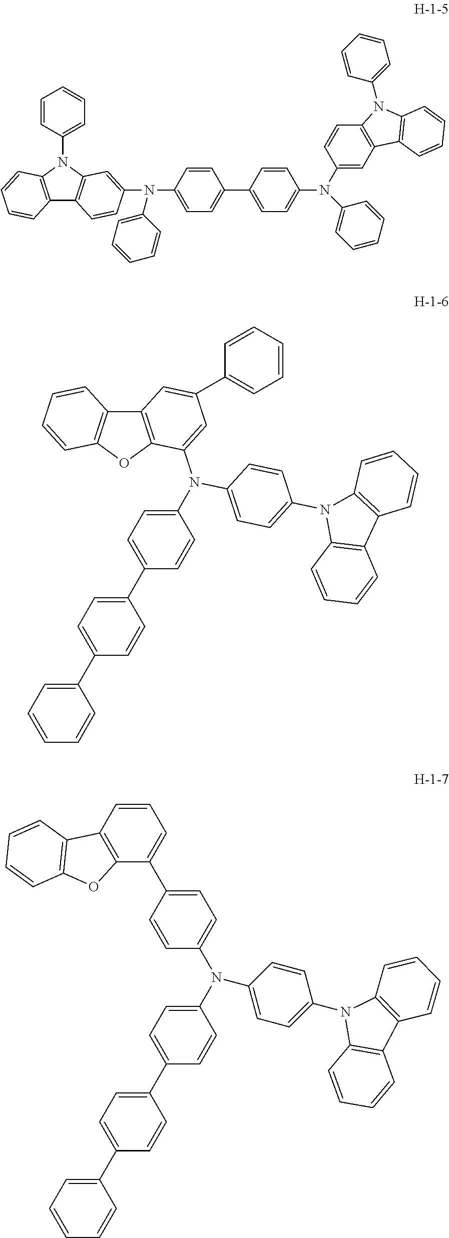 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

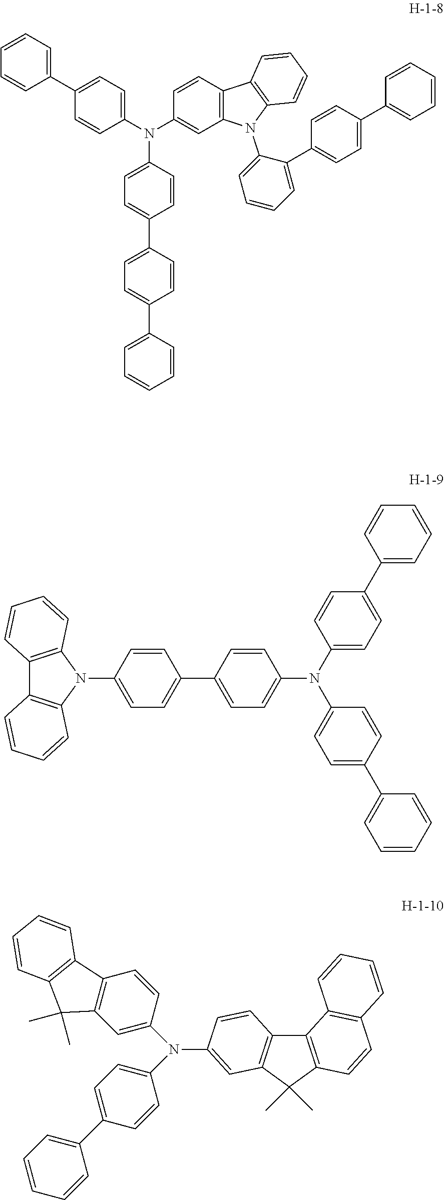 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

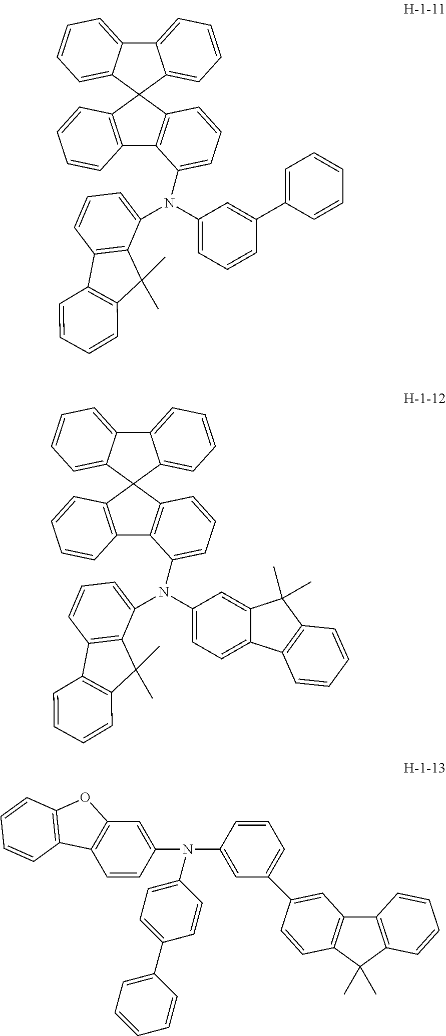 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

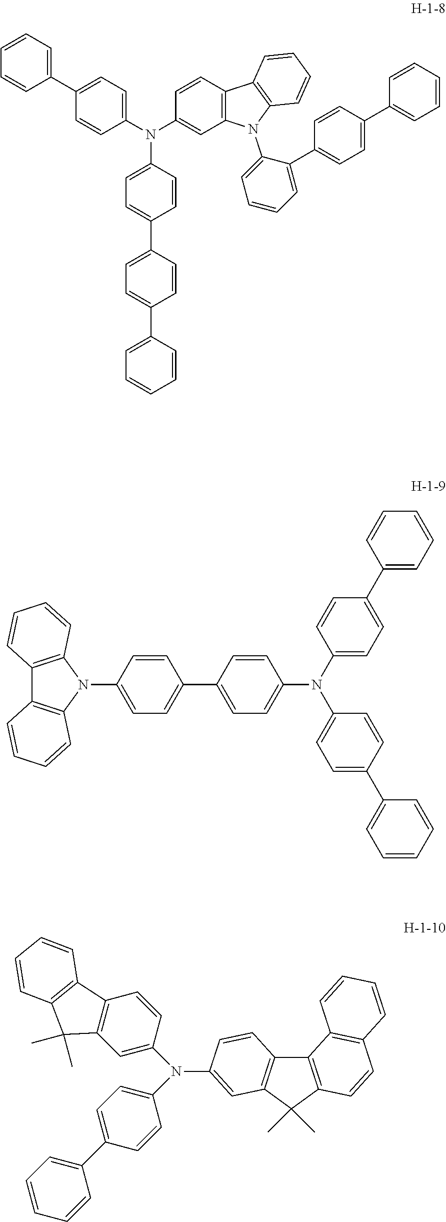 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

The hole transport region HTR may further include a phthalocyanine compound such as copper phthalocyanine, N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonicacid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-Isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HAT-CN), or the like.

The hole transport region HTR may further include carbazole-based derivatives such as N-phenyl carbazole and polyvinyl carbazole, fluorene-based derivatives, N,N′-bis(3-methylphenyl)-N,N′-diphenyl-[1,1′-biphenyl]-4,4′-diamine (TPD), triphenylamine-based derivatives such as 4,4′,4″-tris(N-carbazolyl)triphenylamine (TCTA), N,N′-di(naphthalene-1-yl)-N,N′-diphenyl-benzidine (NPB), 4,4′-Cyclohexylidene bis[N,N-bis(4-methylphenyl)benzenamine] (TAPC), 4,4′-bis[N,N′-(3-tolyl)amino]-3,3′-dimethylbiphenyl (HMTPD), 1,3-bis(N-carbazolyl)benzene (mCP), or the like.

In addition, the hole transport region HTR may further include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), or the like.

The hole transport region HTR may include the compounds of the hole transport region described above in at least one of the hole injection layer HIL, the hole transport layer HTL, the auxiliary emission layer EAL, or the electron blocking layer EBL.

The hole transport region HTR may have a thickness of about 100 Å to about 10000 Å, for example, about 100 Å to about 5000 Å. When the hole transport region HTR includes the hole injection layer HIL, the hole injection layer HIL may have a thickness of, for example, about 30 Å to about 1000 Å. When the hole transport region HTR includes the hole transport layer HTL, the hole transport layer HTL may have a thickness of about 30 Å to about 1000 Å. When the hole transport region HTR includes the electron blocking layer EBL, the electron blocking layer EBL may have a thickness of, for example, about 10 Å to about 1000 Å. When the thicknesses of the hole transport region HTR, the hole injection layer HIL, the hole transport layer HTL, and the electron blocking layer EBL satisfy the above-described ranges, satisfactory hole transport properties may be obtained without a substantial increase in driving voltage.

The hole transport region HTR may further include, in addition to the above-described materials, a charge generation material to increase conductivity. The charge generation material may be uniformly or non-uniformly dispersed in the hole transport region HTR. The charge generation material may be, for example, a p-dopant. The p-dopant may include at least one of halogenated metal compounds, quinone derivatives, metal oxides, or cyano group-containing compounds, but is not limited thereto. For example, the p-dopant may include halogenated metal compounds such as Cul or RbI, quinone derivatives such as tetracyanoquinodimethane (TCNQ) or 2,3,5,6-tetrafluoro-7,7,8,8-tetracyanoquinodimethane (F4-TCNQ), metal oxides such as tungsten oxides or molybdenum oxides, cyano group-containing compounds such as dipyrazino[2,3-f: 2′,3′-h]quinoxaline-2,3,6,7,10,11-hexacarbonitrile (HATCN) or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), or the like, but aspects of the present disclosure are not limited thereto.

As described above, the hole transport region HTR may further include at least one of the auxiliary emission layer EAL or the electron blocking layer EBL, in addition to the hole injection layer HIL and the hole transport layer HTL. The auxiliary emission layer EAL may compensate a resonance distance according to the wavelength of light emitted from the emission layer EML and regulate a hole charge balance to increase luminous efficiency. In addition, the auxiliary emission layer EAL may serve to prevent electrons from being injected into the hole transport region HTR. Materials which may be included in the hole transport region HTR may be included in the auxiliary emission layer EAL. The electron blocking layer EBL is a layer that serves to prevent electrons from being injected from the electron transport region ETR to the hole transport region HTR.

In the light emitting element ED according to an aspect, the emission layer EML is provided on the hole transport region HTR. The emission layer EML may have, for example, a thickness of about 100 Å to about 1000 Å, or about 100 Å to about 300 Å. The emission layer EML may have a single layer formed of a single material, a single layer formed of a plurality of different materials, or a multilayer structure having a plurality of layers formed of a plurality of different materials.

In the light emitting element ED according to an aspect, the emission layer EML may emit blue light. The light emitting element ED according to an aspect includes the amine compound according to an aspect described above in the hole transport region HTR, and may thus exhibit high efficiency and long lifespan in a blue light emitting region. The light emitting element ED according to an aspect may include the amine compound according to an aspect described above in the hole transport region HTR, and the emission layer EML may emit blue fluorescence.

In addition, the emission layer EML of the light emitting element ED according to an aspect may emit light in a wavelength range other than blue light. The light emitting element ED according to an aspect includes the amine compound according to an aspect described above in the hole transport region HTR, and may thus exhibit high efficiency and long lifespan in an emission region of a wavelength range other than blue. The light emitting element ED according to an aspect may include the amine compound according to an aspect described above in the hole transport region HTR, and the emission layer EML emit light of fluorescence. However, aspects of the present disclosure are not limited thereto.

In the light emitting element ED according to an aspect, the emission layer EML may include an anthracene derivative, a pyrene derivative, a fluoranthene derivative, a chrysene derivative, a dihydrobenzanthracene derivative, or a triphenylene derivative. To be specific, the emission layer EML may include an anthracene derivative or a pyrene derivative.

In the light emitting element ED according to an aspect shown in FIGS. 5 to 9, the emission layer EML may include a host and a dopant, and the emission layer EML may include a compound represented by Formula E-1 below. The compound represented by Formula E-1 below may be used as a fluorescent host material.

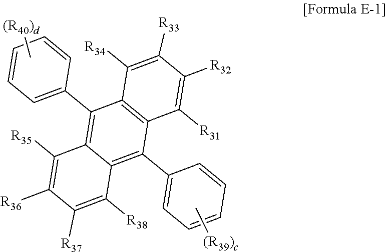

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring. Meanwhile, R₃₁ to R₄₀ may be bonded to an adjacent group to form a saturated hydrocarbon ring, an unsaturated hydrocarbon ring, a saturated heterocycle, or an unsaturated heterocycle.

In Formula E-1, c and d may each independently be an integer of 0 to 5.

Formula E-1 may be represented by any one of compounds E1 to E19 below.

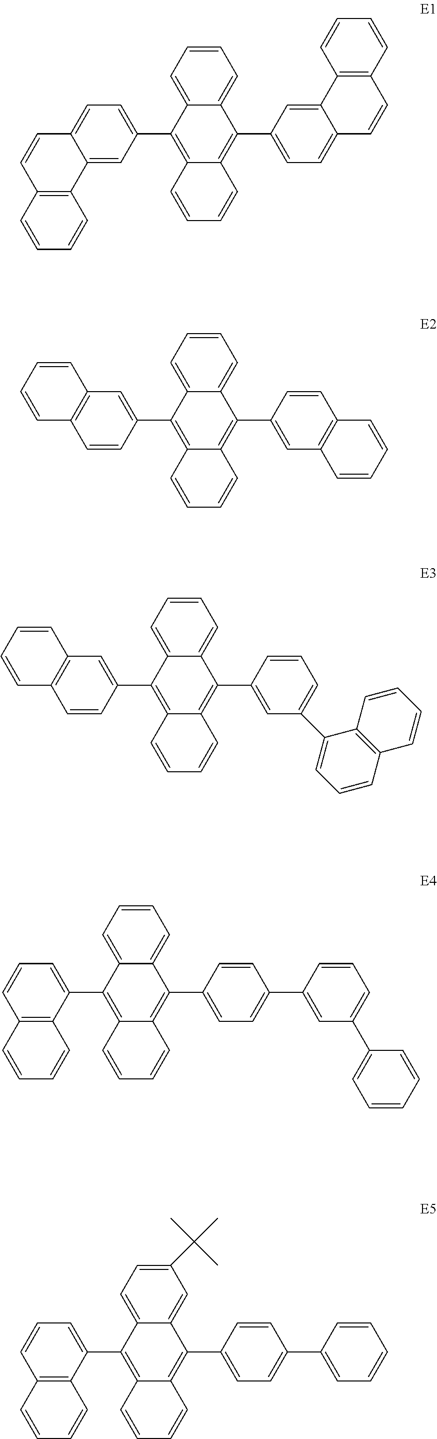

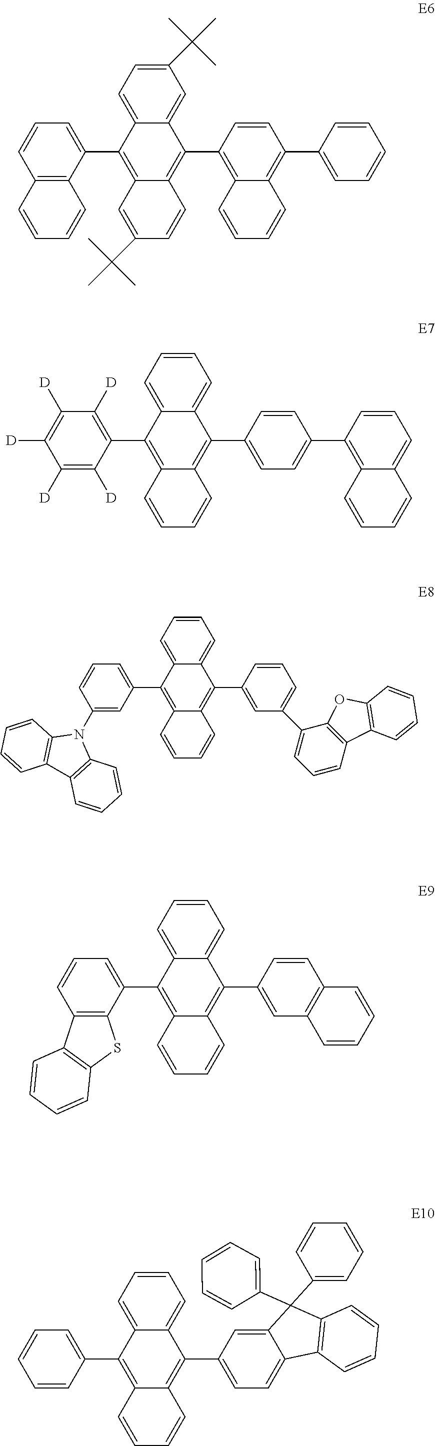

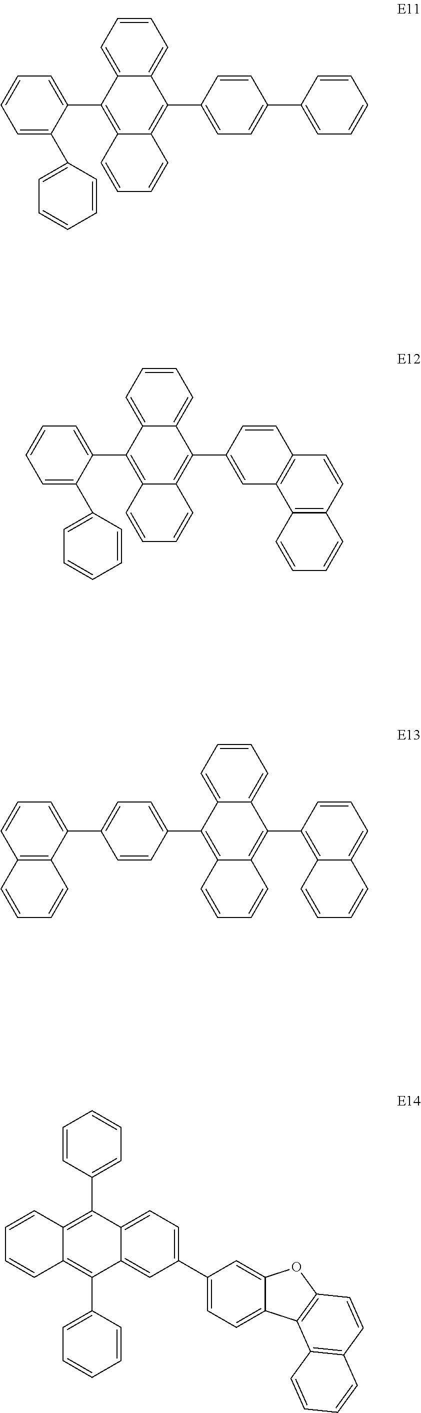

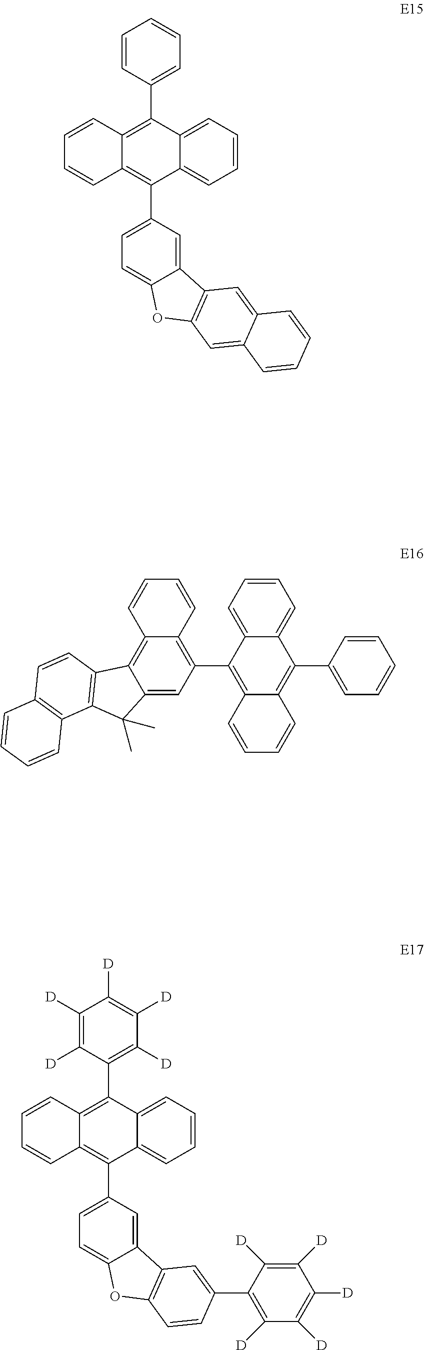

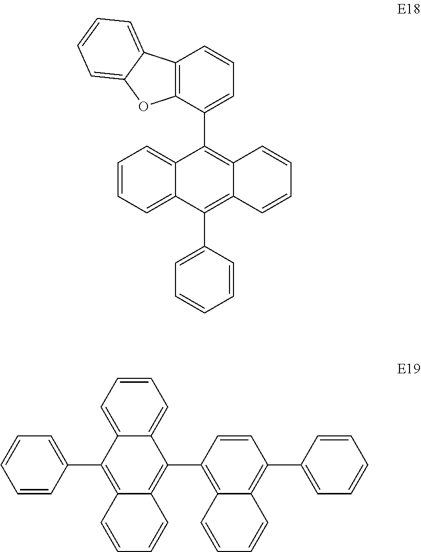

In an aspect, the emission layer EML may further include a compound represented by Formula E-2a or Formula E-2b below. The compound represented by Formula E-2a or Formula E-2b may be used as a phosphorescent host material.

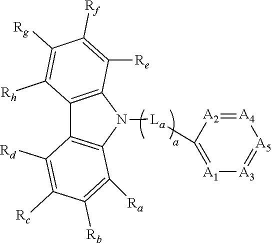

In Formula E-2a, a may be an integer of 0 to 10, and Lₐ may be a direct linkage, a substituted or unsubstituted arylene group having 6 to 30 ring-forming carbon atoms, or a substituted or unsubstituted heteroarylene group having 2 to 30 ring-forming carbon atoms. Meanwhile, when a is an integer of 2 or greater, a plurality of Lₐ's may each independently be a substituted or unsubstituted arylene group having 6 to 30 ring-forming carbon atoms, or a substituted or unsubstituted heteroarylene group having 2 to 30 ring-forming carbon atoms.

In addition, in Formula E-2a, A₁ to A₅ may each independently be N or CRᵢ. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring. Rₐ to Rᵢ may be bonded to an adjacent group to form a hydrocarbon ring or a heterocycle containing N, O, S, or the like as a ring-forming atom.

Meanwhile, in Formula E-2a, two or three selected from A₁ to A₅ may be N, and the others may be CRᵢ.

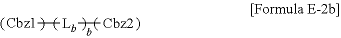

In Formula E-2b, Cbz1 and Cbz2 may each independently be an unsubstituted carbazole group or an aryl-substituted carbazole group having 6 to 30 ring-forming carbon atoms. L_{b} may be a direct linkage, a substituted or unsubstituted arylene group having 6 to 30 ring-forming carbon atoms, or a substituted or unsubstituted heteroarylene group having 2 to 30 ring-forming carbon atoms, b may be an integer of 0 to 10, and when b is an integer of 2 or greater, a plurality of L_{b}'s may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one of compounds from Compound Group E-2 below. However, the compounds listed in Compound Group E-2 below are presented as an example, and the compound represented by Formula E-2a or Formula E-2b is not limited to those listed in Compound Group E-2 below.

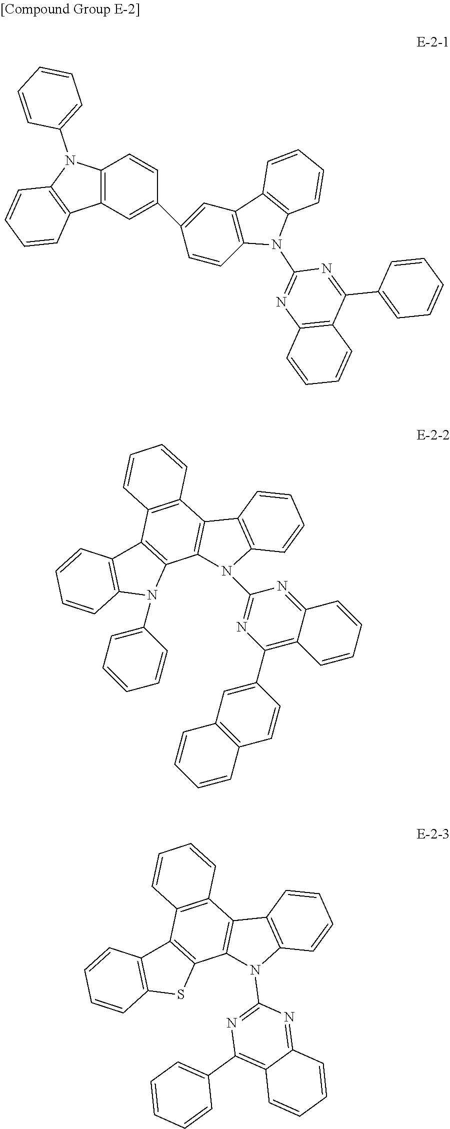 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

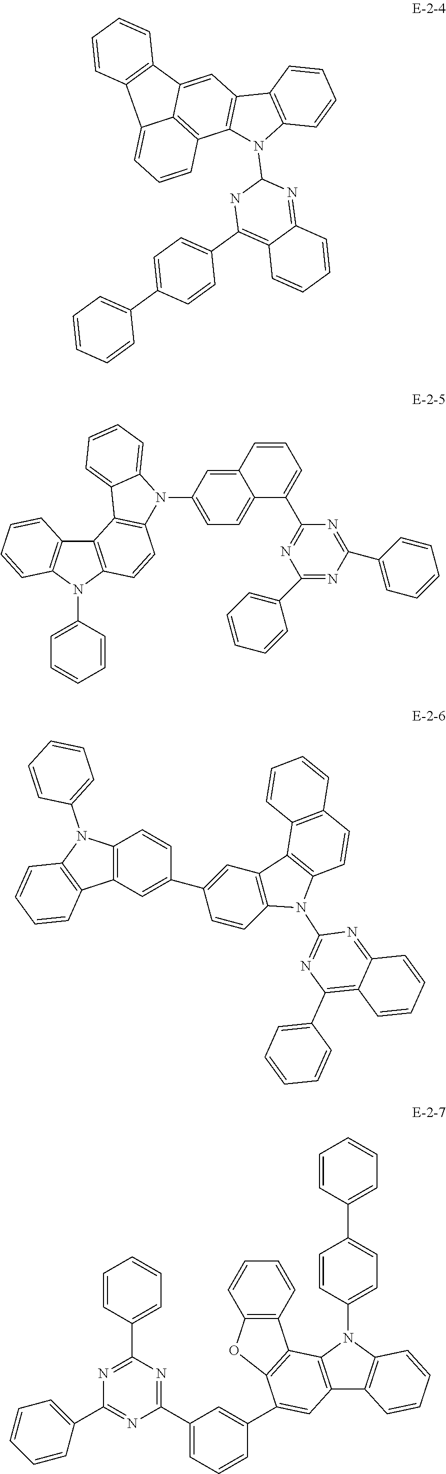 [SMILES: c1ccc(-c2ccc(C3=c4ccccc4=NC(n4c5ccccc5c5cc6c7c(cccc7c54)-c4ccccc4-6)N3)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1]

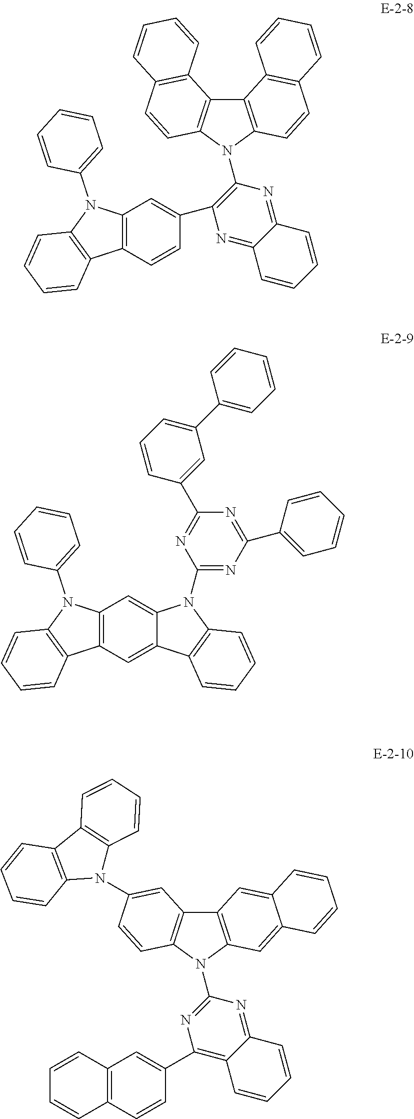 [SMILES: c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1]

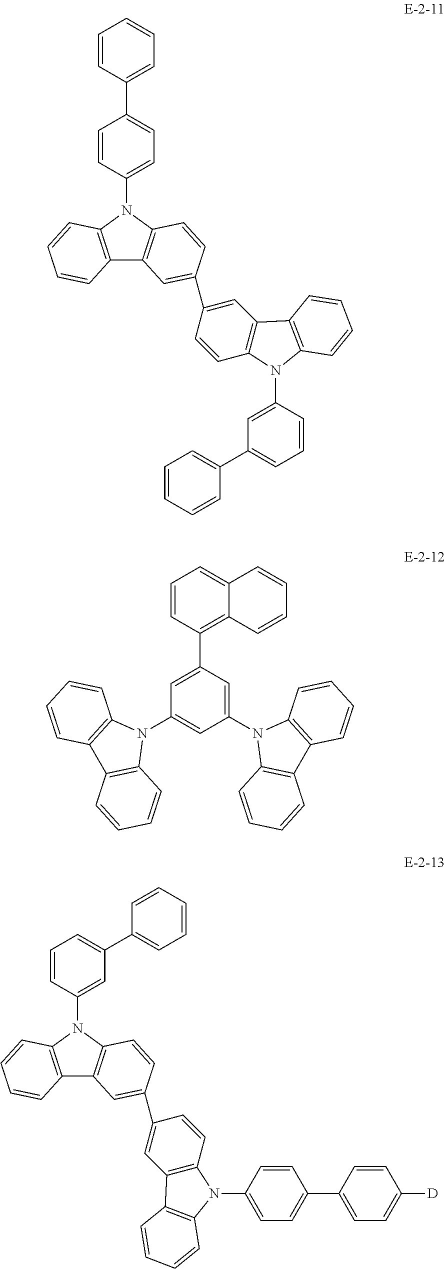 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1]

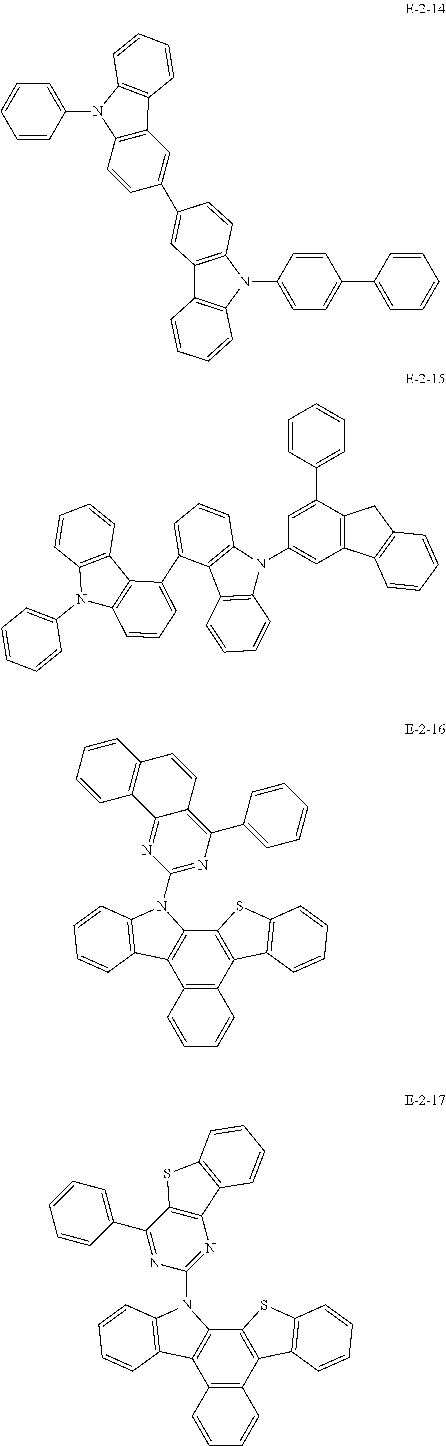 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2Cc2ccccc2-3)cc1 ; c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1]

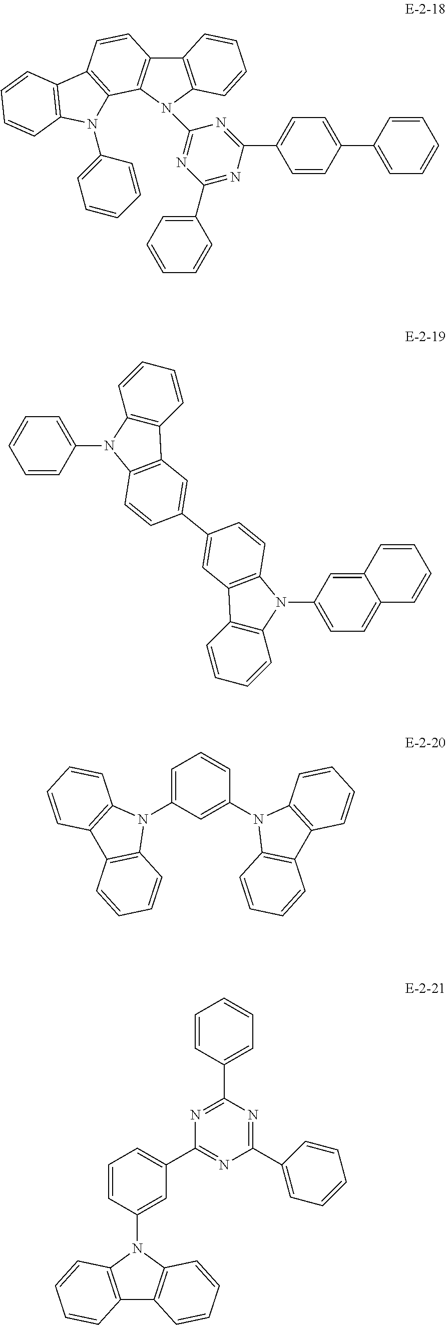 [SMILES: c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1]

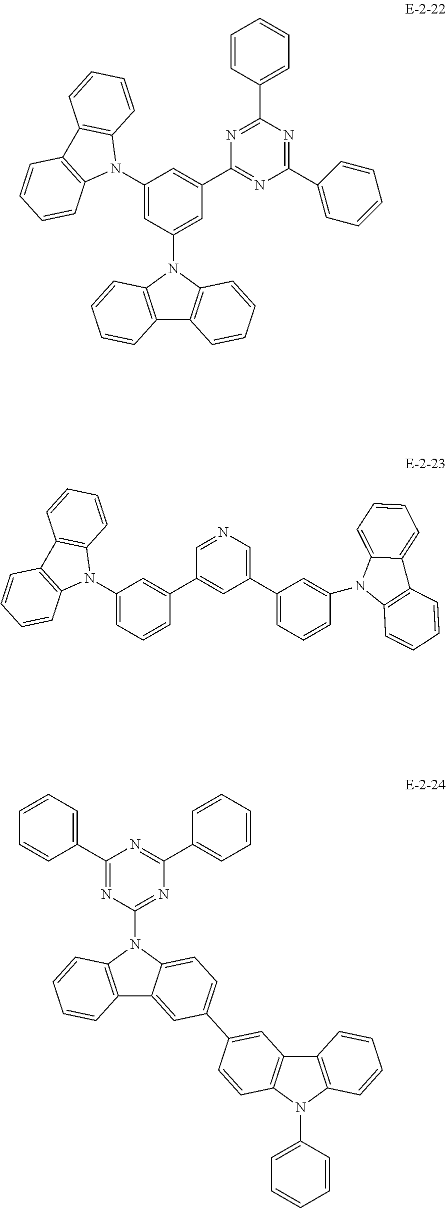 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a general material known in the art as a host material. For example, the emission layer EML may include, as a host material, at least one of bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazolyl-9-yl)benzene (mCP), 2,8-bis(diphenylphosphoryl)dibenzofuran (PPF), 4,4′,4″-tris(carbazol-9-yl)-triphenylamine (TCTA), or 1,3,5-tris(1-phenyl-1H-benzo[d]imidazol-2-yl)benzene (TPBi). However, aspects of the present disclosure are not limited thereto, and for example, tris(8-hydroxyquinolino)aluminum (Alq₃), 9,10-di(naphthalene-2-yl) anthracene (ADN), 3-tert-butyl-9,10-di(naphth-2-yl) anthracene (TBADN), distyrylarylene (DSA), 4,4′-bis(9-carbazolyl)-2,2′-dimethyl-biphenyl (CDBP), 2-methyl-9,10-bis(naphthalen-2-yl) anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetrasiloxane (DPSiO₄), or the like may be used as a host material.

The emission layer EML may include a compound represented by Formula M-a or Formula M-b below. The compound represented by Formula M-a or Formula M-b below may be used as a phosphorescent dopant material.

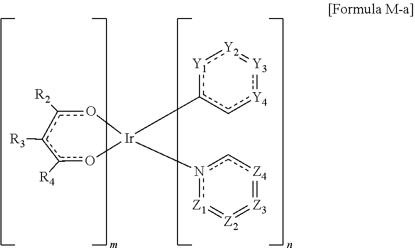

In Formula M-a above, Y₁ to Y₄ and Z₁ to Z₄ may each independently be CR₁ or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring. In Formula M-a, m is 0 or 1, and n is 2 or 3. In Formula M-a, when m is 0, n is 3, and when m is 1, n is 2.

The compound represented by Formula M-a may be represented by any one of compounds M-a1 to M-a25 below. However, the compounds M-a1 to M-a25 below are presented as an example, and the compound represented by Formula M-a is not limited to those represented by the compounds M-a1 to M-a25 below.

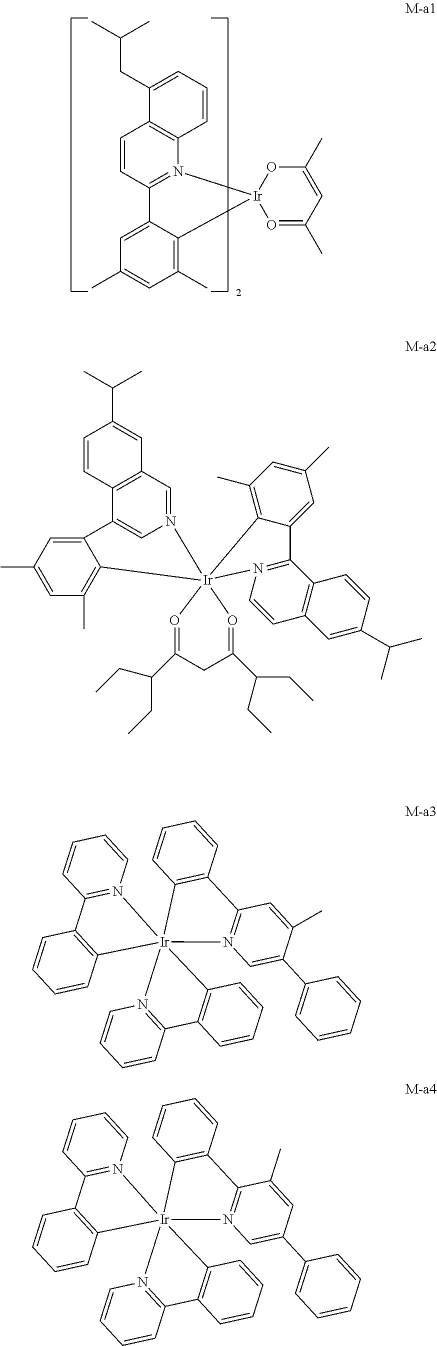

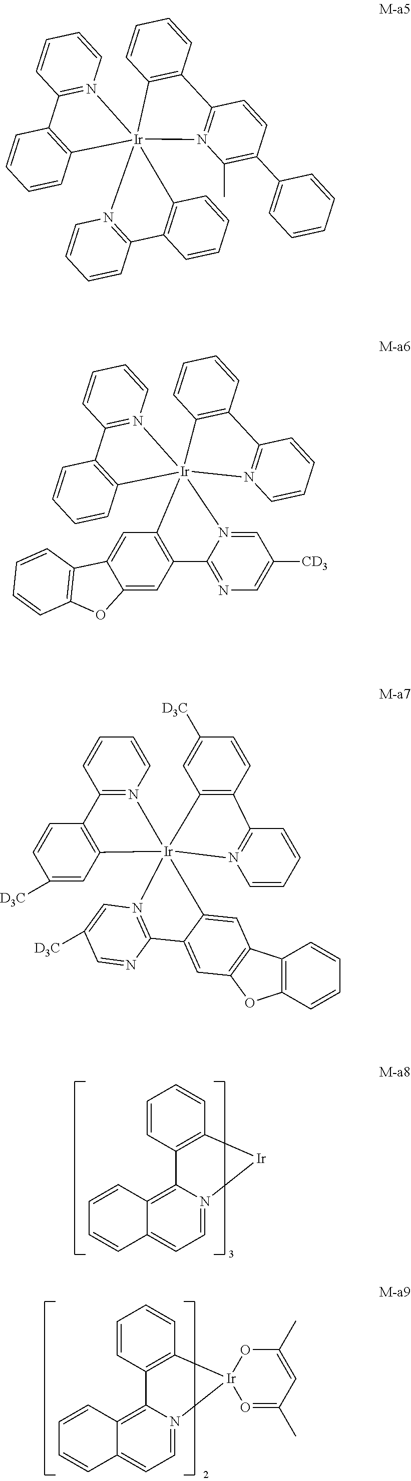

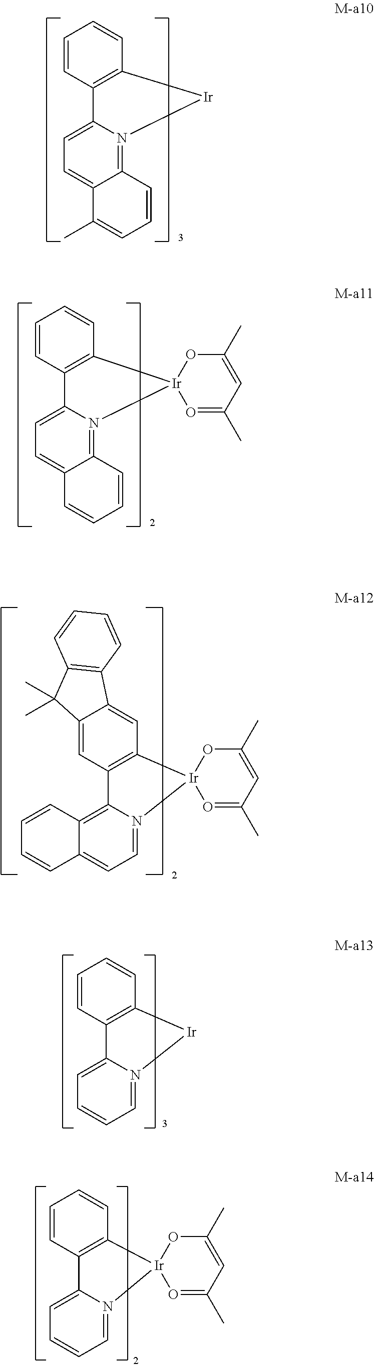

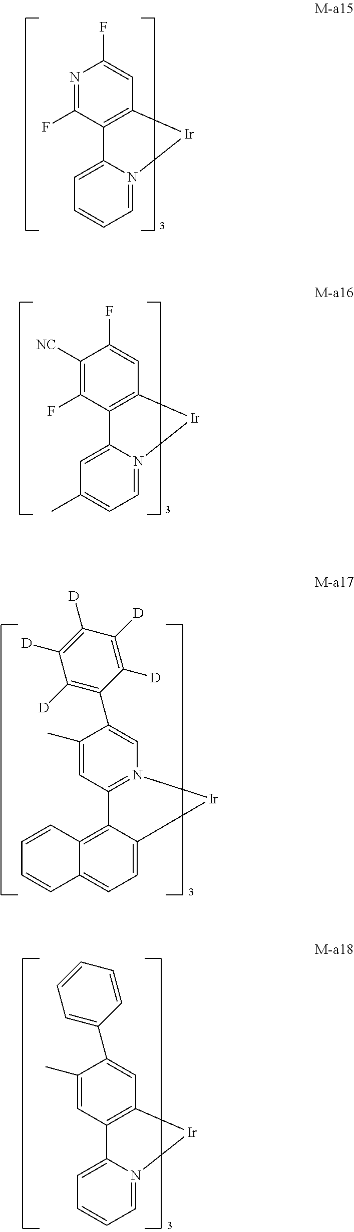

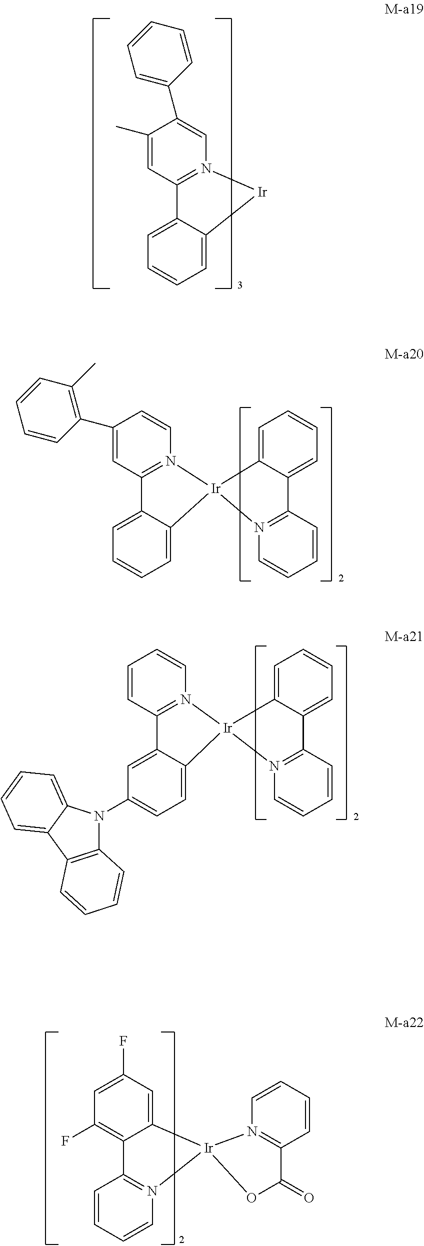

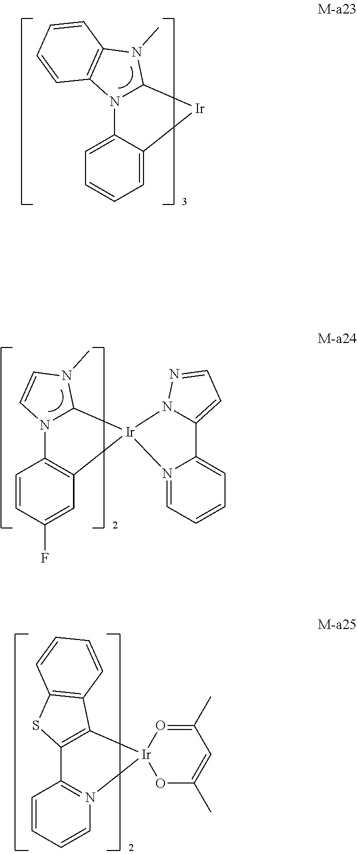

The compounds M-a1 and M-a2 may be used as a red dopant material, and the compounds M-a3 to M-a5 may be used as a green dopant material.

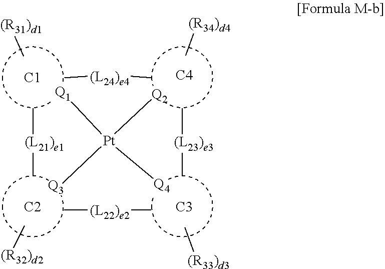

In Formula M-b, Q1 to Q4 are each independently C or N, and C1 to C4 are each independently a substituted or unsubstituted hydrocarbon ring having 5 to 30 ring-forming carbon atoms, or a substituted or unsubstituted heterocycle having 2 to 30 ring-forming carbon atoms. L21 to L24 are each independently a direct linkage,

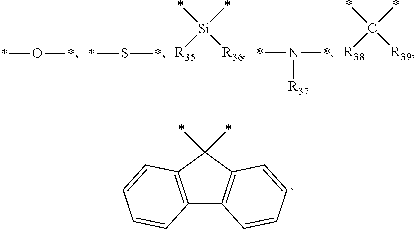

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, and e1 to e4 are each independently 0 or 1. R31 to R39 may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring, and d1 to d4 are each independently an integer of 0 to 4.

A compound represented by Formula M-b may be used as a blue phosphorescent dopant or a green phosphorescent dopant.

The compound represented by Formula M-b may be represented by any one of compounds below. However, the compounds below are presented as an example, and the compound represented by Formula M-b is not limited to those represented by the compounds below.

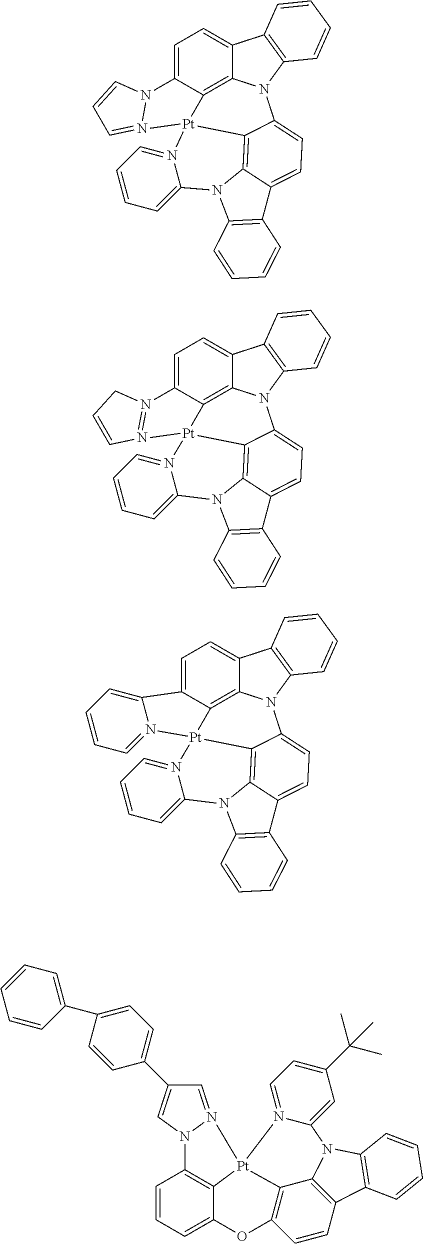

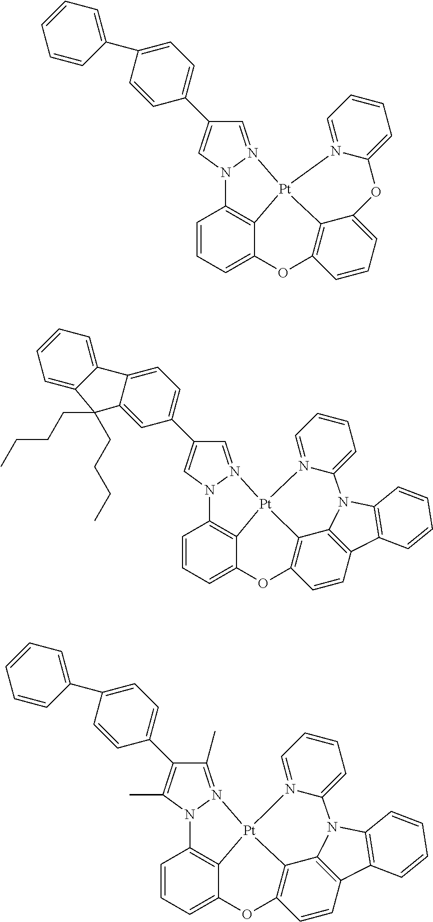

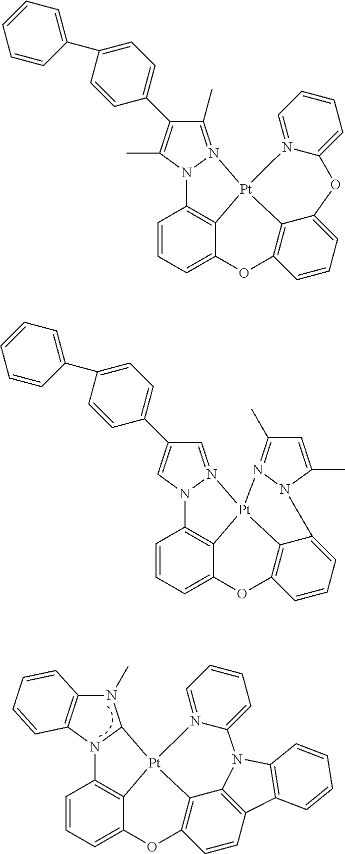

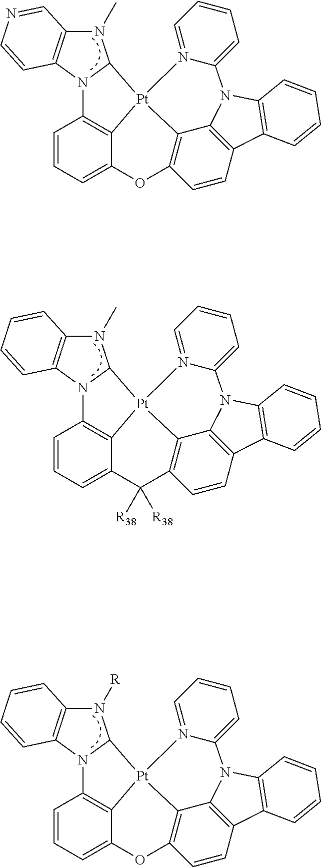

In the compounds above, R, R₃₈, and R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

The emission layer EML may include a compound represented by any one of Formulas F-a to F-c below. The compounds represented by Formulas F-a to F-c below may be used as a fluorescence dopant material.

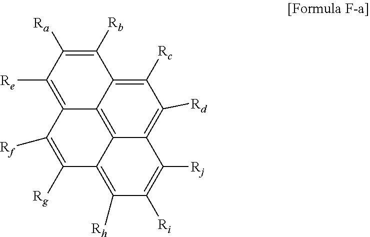

In Formula F-a, two selected from Rₐ to Rⱼ may each independently be substituted with

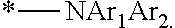

The others among Rₐ to Rⱼ which are not substituted with

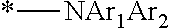

may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In

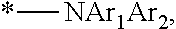

Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one of Ar₁ or Ar₂ may be a heteroaryl group containing O or S as a ring-forming atom.

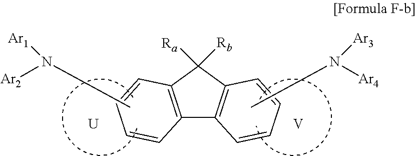

In Formula F-b above, Rₐ and R_{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring. Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms. At least one of Ar₁ to Ar₄ may be a heteroaryl group containing O or S as a ring-forming atom.

In Formula F-b, the number of rings represented by U and V may each independently be 0 or 1. For example, in Formula F-b, when the number of U or Vis 1, one ring forms a fused ring in a portion indicated by U or V, and when the number of U or V is 0, it means that no ring indicated by U or V is present. To be specific, when the number of U is 0 and the number of Vis 1, or when the number of U is 1 and the number of V is 0, a fused ring having a fluorene core of Formula F-b may be a cyclic compound having four rings. In addition, when the number of U and V are both 0, the fused ring of Formula F-b may be a cyclic compound having three rings. In addition, when the number of U and V are both 1, the fused ring having a fluorene core of Formula F-b may be a cyclic compound having five rings.

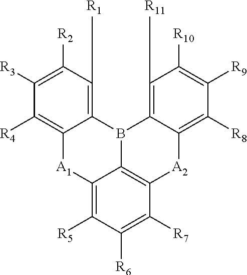

In Formula F-c, A₁ and A₂ may each independently be O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ are each independently a hydrogen atom, a deuterium atom, a halogen atom, a cyano group, a substituted or unsubstituted amine group, a substituted or unsubstituted boron group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or bonded to an adjacent group to form a ring.

In Formula F-c, A₁ and A₂ may each independently be bonded to substituents of neighboring rings to form a fused ring. For example, when A₁ and A₂ are each independently NRₘ, A₁ may be bonded to R₄ or R₅ to form a ring. In addition, A₂ may be bonded to R₇ or R₈ to form a ring.

In an aspect, the emission layer EML may further include, as a known dopant material, styryl derivatives (e.g., 1,4-bis[2-(3-N-ethylcarbazoryl)vinyl]benzene (BCzVB), 4-(di-p-tolylamino)-4′-[(di-p-tolylamino)styryl]stilbene (DPAVB), or N-(4-((E)-2-(6-((E)-4-(diphenylamino)styryl) naphthalen-2-yl)vinyl)phenyl)-N-phenylbenzenamine (N-BDAVBi), 4,4′-bis[2-(4-(N,N-diphenylamino)phenyl)vinyl]biphenyl (DPAVBi), perylene and derivatives thereof (e.g., 2,5,8,11-tetra-t-butylperylene (TBP)), pyrene and derivatives thereof (e.g., 1,1-dipyrene, 1,4-dipyrenylbenzene, 1,4-bis(N,N-diphenylamino)pyrene), or the like.

When the light emitting element is a phosphorescent light emitting element, the emission layer EML may further include a known dopant material. For example, as a phosphorescent dopant, a metal complex including iridium (Ir), platinum (Pt), osmium (Os), gold (Au), titanium (Ti), zirconium (Zr), hafnium (Hf), europium (Eu), and terbium (Tb), or thulium (Tm) may be used. To be specific, iridium (III) bis(4,6-difluorophenylpyridinato-N,C2ç) (FIrpic), bis(2,4-difluorophenylpyridinato)-tetrakis(1-pyrazolyl) borate iridium (III) (Fir6), or platinum octaethyl porphyrin (PtOEP) may be used as a dopant for a phosphorescent light emitting element. However, aspects of the present disclosure are not limited thereto.

Meanwhile, in the light emitting element ED according to an aspect, the emission layer EML may be a delayed fluorescence emission layer including a host and a dopant. To be more specific, the emission layer EML may emit light of thermally activated delayed fluorescence (TADF). In the light emitting element ED according to an aspect, the emission layer EML may include a known thermally activated delayed fluorescence dopant.

In addition, in an aspect, the emission layer EML of the light emitting element ED may include a plurality of host materials, a thermally activated delayed fluorescence dopant, and a phosphorescent sensor.

The emission layer EML may include a quantum dot material. The core of a quantum dot may be selected from a Group II-VI compound, a Group I-II-VI compound, a Group II-IV-VI compound, a Group I-II-IV-VI compound, a Group II-IV-V compound, a Group III-VI compound, a Group I-III-VI element, a Group III-V compound, a Group III-II-V compound, a Group IV-VI compound, a Group IV element, a Group IV compound, and a combination thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a mixture thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and a mixture thereof.

The Group III-VI compound may include a binary compound such as In₂S₃ or In₂Se₃, a ternary compound such as InGaS₃ or InGaSe₃, or any combination thereof.

The Group I-III-VI compound may be selected from a ternary compound selected from the group consisting of AgInS, AgInS₂, CuInS, CuInS₂, AgGaS₂, CuGaS₂CuGaO₂, AgGaO₂, AgAlO₂, and a mixture thereof, or a quaternary compound such as AgInGaS₂ or CuInGaS₂.

The Group III-V compound may be selected from the group consisting of a binary compound selected from the group consisting of GaN, GaP, GaAs, GaSb, AlN, AlP, AlAs, AlSb, InN, InP, InAs, InSb, and a mixture thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and a mixture thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and a mixture thereof. Meanwhile, the Group III-V compound may further include a Group II metal. For example, InZnP or the like may be selected as a Group III-II-V compound.

The Group IV-VI compound may be selected from the group consisting of a binary compound selected from the group consisting of SnS, SnSe, SnTe, PbS, PbSe, PbTe, and a mixture thereof, a ternary compound selected from the group consisting of SnSeS, SnSeTe, SnSTe, PbSeS, PbSeTe, PbSTe, SnPbS, SnPbSe, SnPbTe, and a mixture thereof, and a quaternary compound selected from the group consisting of SnPbSSe, SnPbSeTe, SnPbSTe, and a mixture thereof. The Group IV element may be selected from the group consisting of Si, Ge, and a mixture thereof. The Group IV compound may be a binary compound selected from the group consisting of SiC, SiGe, and a mixture thereof.

Each element included in the multi-element compound such as the binary compound, ternary compound, and quaternary compound may be present in particles at a uniform concentration or a non-uniform concentration. That is, Formula above indicates the types of elements included in a compound, and element ratios in the compound may be different. For example, AgInGaS₂ may indicate AgInₓGa₁₋ₓS₂ (x is a real number between 0 and 1).

Meanwhile, the quantum dot may have a single structure having a uniform concentration of each element included in the corresponding quantum dot or a dual structure of core-shell. For example, materials included in the core may be different from materials included in the shell.

The shell of the quantum dot may serve as a protection layer to prevent the chemical deformation of the core so as to keep semiconductor properties, and/or a charging layer to impart electrophoresis properties to the quantum dot. The shell may be single-layered or multi-layered. An interface between the core and the shell may have a concentration gradient in which the concentration of an element present in the shell becomes lower towards the center.

Examples of the shell of the quantum dot may be a metal or non-metal oxide, a semiconductor compound, or a combination thereof. For example, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, NiO, or a ternary compound such as MgAl₂O₄, CoFe₂O₄, NiFe₂O₄, and CoMn₂O₄, but aspects of the present disclosure are not limited thereto.

In addition, the semiconductor compound may be, for example, CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or the like, but aspects of the present disclosure are not limited thereto.

Each element included in the multi-element compound such as the binary compound and the ternary compound may be present in particles at a uniform concentration or a non-uniform concentration. That is, Formula above indicates the types of elements included in a compound, and element ratios in the compound may be different.

The quantum dot may have, in a light emitting wavelength spectrum, a full width of half maximum (FWHM) of about 45 nm or less, or about 40 nm or less, or about 30 nm or less, and in this range, color purity or color reproducibility may be improved. In addition, light emitted through the quantum dot is emitted in all directions, and thus a wide viewing angle may be improved.

In addition, the form of the quantum dot is not particularly limited as long as it is a form commonly used in the art, but more specifically, a quantum dot in the form of spherical, pyramidal, multi-arm, or cubic nanoparticles, nanotubes, nanowires, nanofibers, nanoplatelets, or the like may be used.

As the size of the quantum dot or the ratio of elements in the quantum dot compound is regulated, the energy band gap may be accordingly controlled to obtain light of various wavelengths from the quantum dot emission layer. Therefore, by using the quantum dots as described above (using quantum dots of different sizes or having different element ratios in the quantum dot compound), a light emitting element emitting light of various wavelengths may be obtained. Specifically, the size of the quantum dot or the ratio of elements in the quantum dot compound may be regulated to emit red, green, and/or blue light. In addition, the quantum dots may be configured to emit white light by combining light of various colors.

In the light emitting element ED according to an aspect shown in FIGS. 5 to 9, an electron transport region ETR is provided on the emission layer EML. The electron transport region ETR may include at least one among a hole blocking layer HBL, an electron transport layer ETL, and an electron injection layer EIL, but aspects of the present disclosure are not limited thereto.

The electron transport region ETR may have a single layer formed of a single material, a single layer formed of a plurality of different materials, or a multilayer structure having a plurality of layers formed of a plurality of different materials.

For example, the electron transport region ETR may have a single layer structure of an electron injection layer EIL or an electron transport layer ETL, and may have a single layer structure formed of an electron injection material and an electron transport material. In addition, the electron transport region ETR may have a single layer structure formed of a plurality of different materials, or may have a structure in which an electron transport layer ETL/electron injection layer EIL, or a hole blocking layer HBL/electron transport layer ETL/electron injection layer EIL are stacked in order from the emission layer EML, but is not limited thereto. The electron transport region ETR may have a thickness of, for example, about 1000 Å to about 1500 Å.

The electron transport region ETR may be formed using various methods such as a vacuum deposition method, a spin coating method, a cast method, a Langmuir-Blodgett (LB) method, an inkjet printing method, a laser printing method, or a laser induced thermal imaging (LITI) method.

The electron transport region ETR may include a compound represented by Formula ET-2 below.

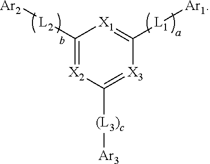

In Formula ET-2, at least one of X₁ to X₃ is N and the others are Crₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-2, a to c may each independently be an integer of 0 to 10. In Formula ET-2, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. Meanwhile, when a to c are an integer of 2 or greater, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, aspects of the present disclosure are not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl) biphenyl-3-yl)-1,3,5-triazine, 2-(4-(N-phenylbenzoimidazolyl-1-ylphenyl)-9,10-dinaphthylanthracene, 1,3,5-tri (1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl) anthracene (ADN), 1,3-bis[3,5-di(pyridin-3-yl)phenyl]benzene (BmPyPhB), or a mixture thereof.

The electron transport region ETR may include at least one of compounds ET1 to ET36 below.

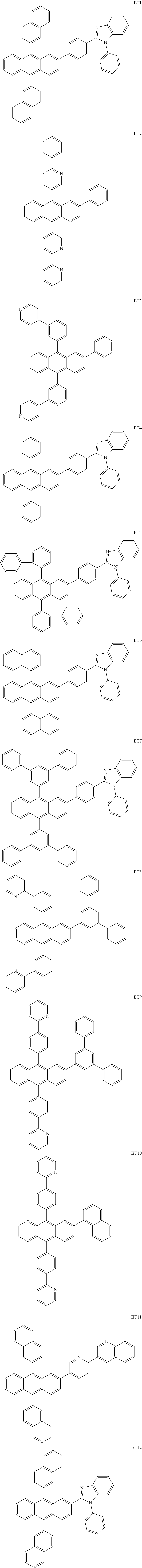

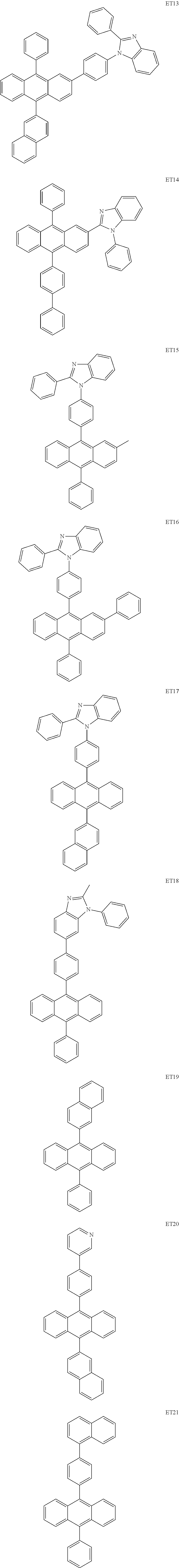

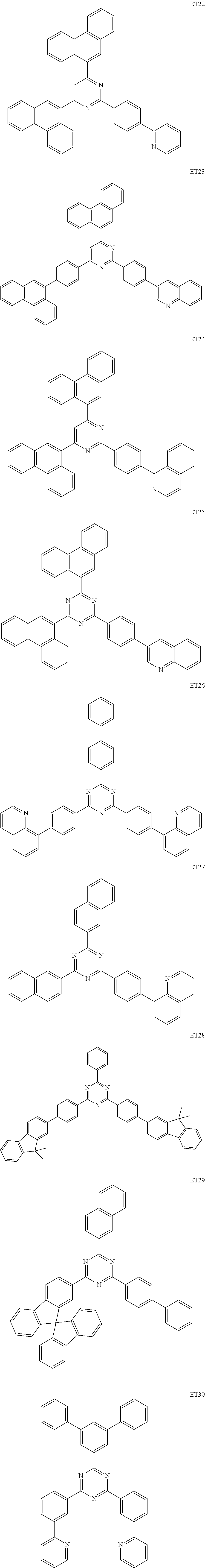

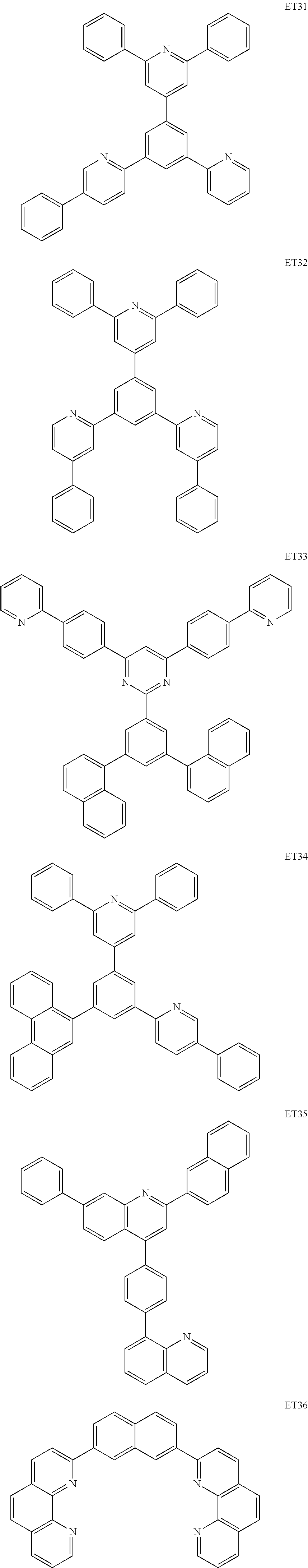

In addition, the electron transport region ETR may include halogenated metals such as LiF, NaCl, CsF, RbCl, RbI, Cul, or KI, lanthanide metals such as Yb, or co-deposition materials of a halogenated metal and a lanthanide metal. For example, the electron transport region ETR may include KI:Yb, RbI:Yb, LiF:Yb, or the like as a co-deposition material. Meanwhile, for the electron transport region ETR, a metal oxide such as Li₂O or BaO, 8-hydroxyl-lithium quinolate (Liq), or the like may be used, but aspects of the present disclosure are limited thereto. The electron transport region ETR may also be formed of a mixture material of an electron transport material and an insulating organo-metal salt. The organo metal salt may be a material having an energy band gap of about 4 eV or greater. Specifically, the organo-metal salt may include, for example, metal acetates, metal benzoates, metal acetoacetates, metal acetylacetonates, or metal stearates.

The electron transport region ETR may further include, for example, at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) in addition to the materials described above, but aspects of the present disclosure are not limited thereto.

The electron transport region ETR may include the compounds of the electron transport region described above in at least one among the electron injection layer EIL, the electron transport layer ETL, and the hole blocking layer HBL.

When the electron transport region ETR includes the electron transport layer ETL, the electron transport layer ETL may have a thickness of about 100 Å to about 1000 Å, for example, about 150 Å to about 500 Å. When the thickness of the electron transport layer ETL satisfies the above-described range, satisfactory electron transport properties may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. When the thickness of the electron injection layer EIL satisfies the above-described ranges, satisfactory electron injection properties may be obtained without a substantial increase in driving voltage.

The second electrode EL2 is provided on the electron transport region ETR. The second electrode EL2 may be a common electrode. The second electrode EL2 may be a cathode or an anode but aspects of the present disclosure are not limited thereto. For example, when the first electrode EL1 is an anode, the second electrode EL2 may be a cathode, and when the first electrode EL1 is a cathode, the second electrode EL2 may be an anode.

The second electrode EL2 may be a transmissive electrode, a transflective electrode, or a reflective electrode. When the second electrode EL2 is a transmissive electrode, the second electrode EL2 may be formed of a transparent metal oxide, for example, indium tin oxide (ITO), indium zinc oxide (IZO), zinc oxide (ZnO), indium tin zinc oxide (ITZO), or the like.

When the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, a compound thereof, or a mixture thereof (e.g., AgMg, AgYb, or MgYb). Alternatively, the second electrode EL2 may have a multilayer structure including a reflective film or a transflective film formed of the above-described materials, and a transparent conductive film formed of indium tin oxide (ITO), indium zinc oxide (IZO), zinc oxide (ZnO), indium tin zinc oxide (ITZO), or the like. For example, the second electrode EL2 may include the above-described metal materials, a combination of two or more metal materials selected from the above-described metal materials, or oxides of the above-described metal materials.

Although not shown, the second electrode EL2 may be connected with an auxiliary electrode. When the second electrode EL2 is connected with the auxiliary electrode, the resistance of the second electrode EL2 may decrease.

Meanwhile, a capping layer CPL may be further disposed on the second electrode EL2 of the light emitting element ED according to an aspect. The capping layer CPL may include a multi-layer or a single layer.

In an aspect, the capping layer CPL may be an organic layer or an inorganic layer. For example, when the capping layer CPL includes an inorganic material, the inorganic material may include an alkali metal compound such as LiF, an alkaline earth metal compound such as MgF₂, SiON, SiN_{X}, SiOy, or the like.

For example, when the capping layer CPL includes an organic material, the organic material may include α-NPD, NPB, TPD, m-MTDATA, Alq₃CuPc, N4,N4,N4′,N4′-tetra(biphenyl-4-yl) biphenyl-4,4′-diamine (TPD15), 4,4′,4″-tris(carbazol sol-9-yl)triphenylamine (TCTA), or the like, or may include epoxy resins or acrylates such as methacrylates. However, aspects of the present disclosure are not limited thereto, and the capping layer CPL may include at least one of compounds P1 to P5 below.

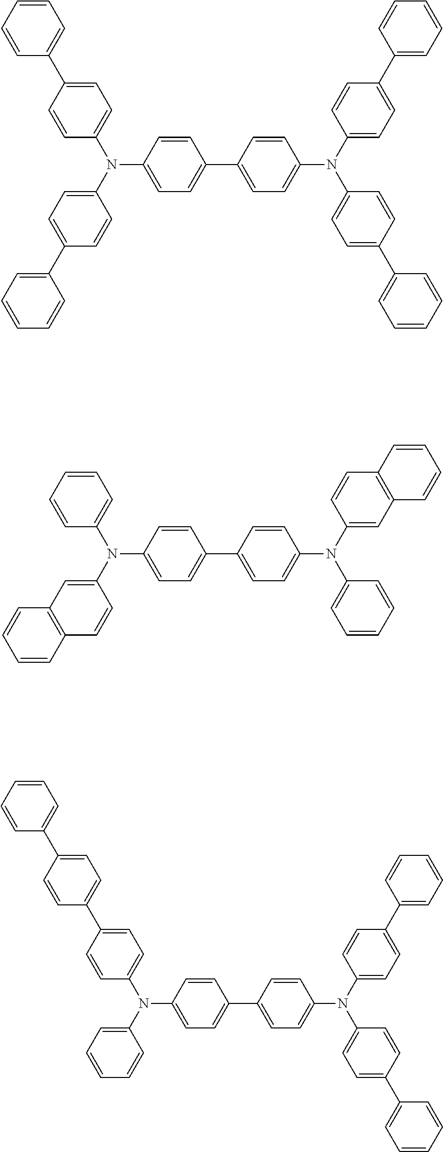 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

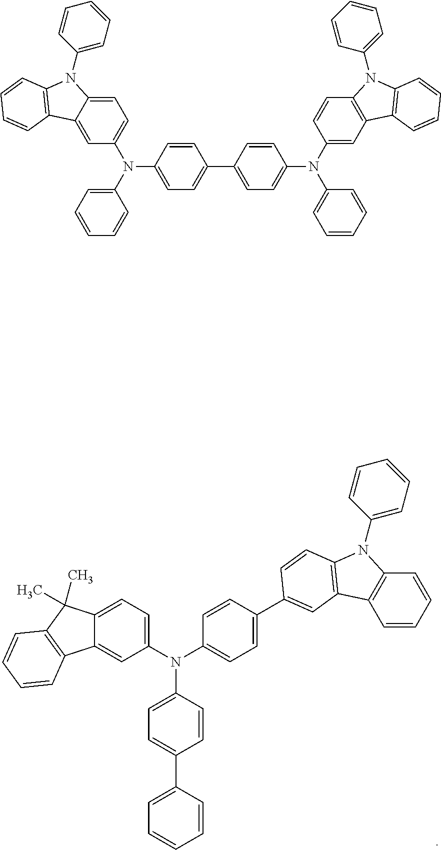 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

Meanwhile, the capping layer CPL may have a refractive index of about 1.6 or greater. To be specific, the capping layer CPL may have a refractive index of about 1.6 or greater in a wavelength range of about 550 nm to about 660 nm.

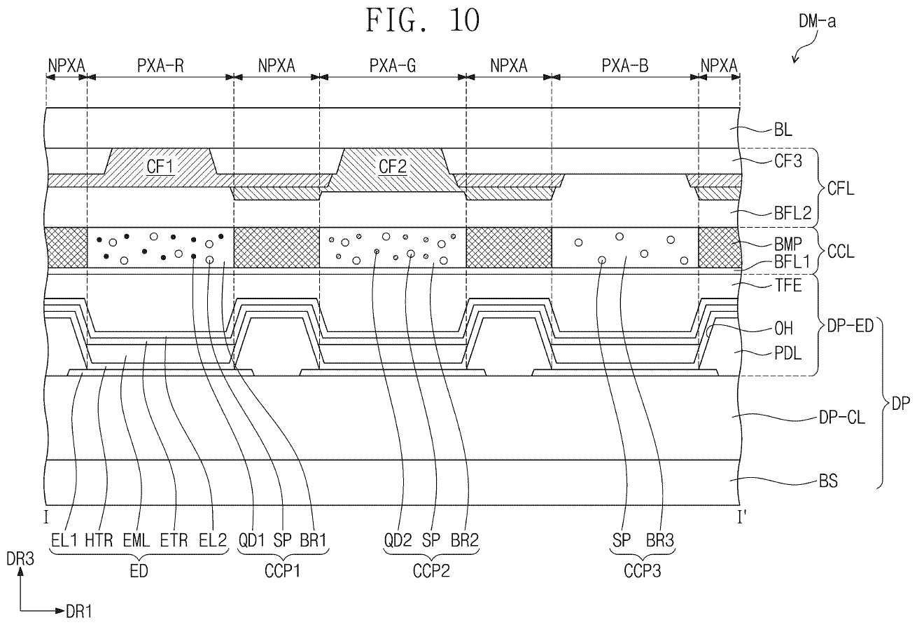

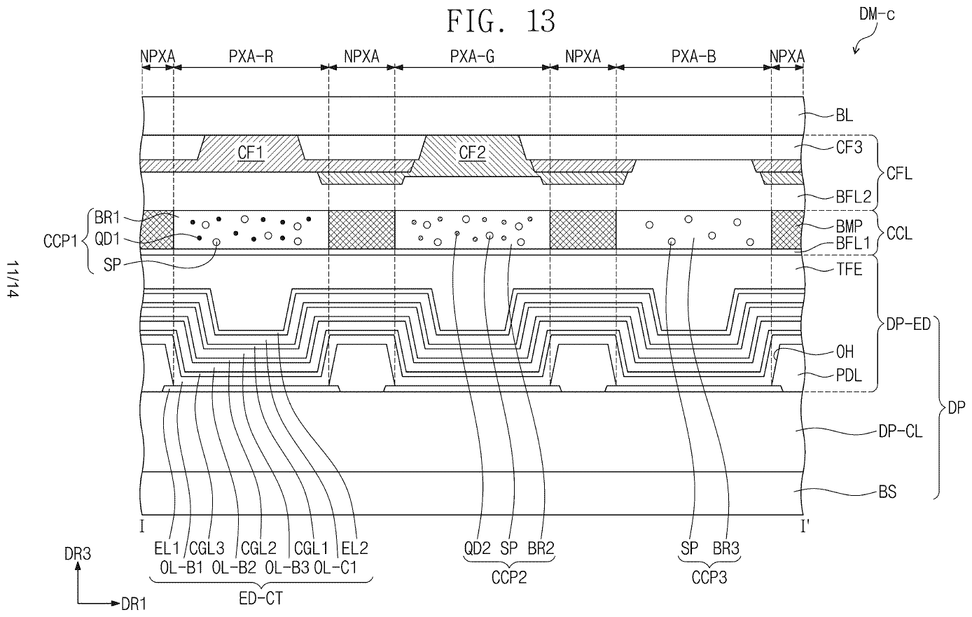

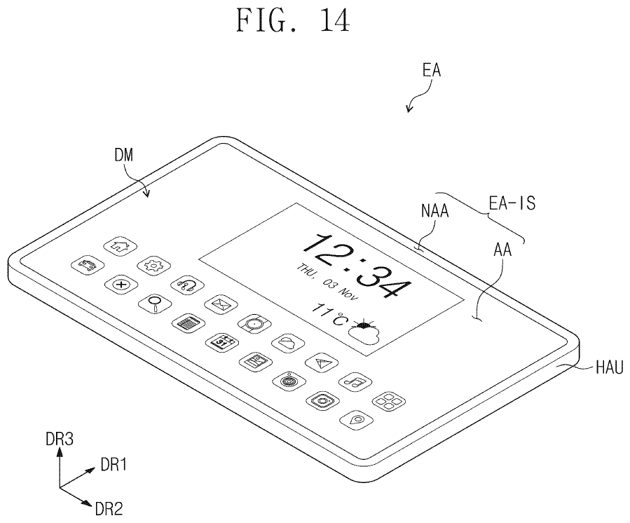

FIGS. 10 to 13 are each a cross-sectional view of a display module according to an aspect. Hereinafter, in the description of the display module according to an aspect with reference to FIGS. 10 and 13, content overlapping the one described above with reference to FIGS. 1 to 9 will not be described again, and the differences will be mainly described.

Referring to FIG. 10, a display module DM-a according to an aspect may include a display panel DP having a display element layer DP-ED, a light control layer CCL disposed on the display panel DP, and a color filter layer CFL. In an aspect shown in FIG. 10, the display panel DP may include a base layer BS, a circuit layer DP-CL provided on the base layer BS, and a display element layer DP-ED, and the element layer DP-ED may include a light emitting element ED.

The light emitting element ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. A structure of the light emitting element ED shown in FIG. 10 may be the same as the structure of the light emitting element of FIGS. 5 to 9 described above. The light emitting element ED shown in FIG. 10 may include the amine compound according to an aspect. The light emitting element ED according to an aspect may include the amine compound according to an aspect in the hole transport region HTR, and may thus exhibit long lifespan characteristics. In addition, the light emitting element ED according to an aspect may emit blue fluorescence and exhibit long lifespan characteristics.

Referring to FIG. 10, the emission layer EML may be disposed in the openings OH defined in the pixel defining films PDL. For example, the emission layer EML separated by the pixel defining films PDL and provided corresponding to each of light emitting regions PXA-R, PXA-G, and PXA-B may emit light in the same wavelength ranges. In the display module DM-a according to an aspect, the emission layer EML may emit blue light. Unlike what is shown, in an aspect, the emission layer EML may be provided as a common layer throughout the light emitting regions PXA-R, PXA-G, and PXA-B.

The light control layer CCL may be disposed on the display panel DP. Although the light control layer CCL is shown to be disposed on an upper side of the display element layer DP-ED, aspects of the present disclosure are not limited thereto, and the light control layer CCL may be disposed on a lower side of the display element layer DP-ED. The light control layer CCL may include a light converter. The light converter may be a quantum dot or a phosphor. The light converter may wavelength-convert the provided light and emit the wavelength-converted light. That is, the light control layer CCL may be a layer containing quantum dots or phosphors.

The light control layer CCL may include a plurality of light control units CCP1, CCP2, and CCP3. The light control units CCP1, CCP2, and CCP3 may be spaced apart from one another.

Referring to FIG. 10, a division pattern BMP may be disposed between the light control units CCP1, CCP2, and CCP3 spaced apart from each other, but aspects of the present disclosure are not limited thereto. In FIG. 10, the division pattern BMP is shown to nonoverlap the light control units CCP1, CCP2, and CCP3, but edges of the light control units CCP1, CCP2, and CCP3 may overlap at least a portion of the division pattern BMP.

The light control layer CCL may include a first light control unit CCP1 including a first quantum dot QD1 for converting first color light provided from the light emitting element ED into second color light, a second light control unit CCP2 including a second quantum dot QD2 for converting the first color light into third color light, and a third light control unit CCP3 transmitting the first color light.

In an aspect, the first light control unit CCP1 may provide red light, which is the second color light, and the second light control unit CCP2 may provide green light, which is the third color light. The third light control unit CCP3 may transmit and provide blue light, which is the first color light provided from the light emitting element ED. For example, the first quantum dot QD1 may be a red quantum dot and the second quantum dot QD2 may be a green quantum dot. The same descriptions above may be applied to the quantum dots QD1 and QD2.

In addition, the light control layer CCL may further include scatterers SP. The first light control unit CCP1 may include the first quantum dot QD1 and the scatterers SP, the second light control unit CCP2 may include the second quantum dot QD2 and the scatterers SP, and the third light control unit CCP3 may not include a quantum dot but may include the scatterers SP.

The scatterers SP may be inorganic particles. For example, the scatterers SP may include at least one among TiO₂, ZnO, Al₂O₃, SiO₂, and hollow silica. The scatterers SP may include any one of TiO₂, ZnO, Al₂O₃, SiO₂, and hollow silica, or may be a mixture of two or more materials selected from TiO₂, ZnO, Al₂O₃, SiO₂, and hollow silica.

The first light control unit CCP1, the second light control unit CCP2, and the third light control unit CCP3 may each include base resins BR1, BR2, and BR3 for dispersing the quantum dots QD1 and QD2 and the scatterers SP. In an aspect, the first light control unit CCP1 may include the first quantum dot QD1 and the scatterers SP dispersed in the first base resin BR1, the second light control unit CCP2 may include the second quantum dot QD2 and the scatterers SP dispersed in the second base resin BR2, and the third light control unit CCP3 may include the scatterers SP dispersed in the third base resin BR3.

The base resins BR1, BR2, and BR3 are a medium in which the quantum dots QD1 and QD2 and the scatterers SP are dispersed, and may be formed of various resin compositions, which may be generally referred to as a binder. For example, the base resins BR1, BR2, and BR3 may be an acrylic-based resin, a urethane-based resin, a silicone-based resin, an epoxy-based resin, or the like. Base resins BR1, BR2, and BR3 may be transparent resins. In an aspect, the first base resin BR1, the second base resin BR2, and the third base resin BR3 may each be the same as or different from each other.

The light control layer CCL may include a barrier layer BFL1. The barrier layer BFL1 may serve to prevent moisture and/or oxygen (hereinafter referred to as “moisture/oxygen”) from being introduced. The barrier layer BFL1 may be disposed on the light control units CCP1, CCP2, and CCP3 to prevent the light control units CCP1, CCP2, and CCP3 from being exposed to moisture/oxygen. The barrier layer BFL1 may cover the light control units CCP1, CCP2, and CCP3. In addition, a barrier layer BFL2 may be provided between the light control units CCP1, CCP2, and CCP3 and the color filter layer CFL.

The barrier layers BFL1 and BFL2 may include at least one inorganic layer. That is, the barrier layers BFL1 and BFL2 may be formed of an inorganic material. For example, the barrier layers BFL1 and BFL2 may be formed including silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, or a metal thin film in which light transmittance is secured, or the like. The barrier layers BFL1 and BFL2 may further include an organic film. The barrier layers BFL1 and BFL2 may be formed of a single layer or a plurality of layers.

In the display module DM-a according to an aspect, the color filter layer CFL may be disposed on the light control layer CCL. For example, the color filter layer CFL may be directly disposed on the light control layer CCL. In this case, the barrier layer BFL2 may be omitted.

The color filter layer CFL may include filters CF1, CF2, and CF₃. That is, the color filter layer CFL may include a first filter CF1 transmitting second color light, a second filter CF2 transmitting third color light, and a third filter CF3 transmitting first color light. For example, the first filter CF1 may be a red filter, the second filter CF2 may be a green filter, and the third filter CF3 may be a blue filter. The filters CF1, CF2, and CF3 may each include a polymer photosensitive resin, a pigment or a dye. The first filter CF1 may include a red pigment or a red dye, the second filter CF2 may include a green pigment or a green dye, and the third filter CF3 may include a blue pigment or a blue dye.

An aspect is not limited thereto, and the third filter CF3 may not include a pigment or a dye. The third filter CF3 may include a polymer photosensitive resin, but not include a pigment or a dye. The third filter CF3 may be transparent. The third filter CF3 may be formed of a transparent photosensitive resin.

In addition, in an aspect, the first filter CF1 and the second filter CF2 may be yellow filters. The first filter CF1 and the second filter CF2 may not be separated and may be provided as a single body.

Although not shown, the color filter layer CFL may further include a light blocking unit (not shown). The light blocking unit may be a black matrix. The light blocking unit may be formed including an organic light blocking material or an inorganic light blocking material, both including a black pigment or a black dye. The light blocking unit may prevent light leakage, and separate boundaries between the adjacent filters CF1, CF2, and CF3. In addition, in an aspect, the light blocking unit may be formed with a blue filter.

The first to third color filters CF1, CF2, and CF3 may respectively correspond to the first light emitting region PXA-R, the second light emitting region PXA-G, and the third light emitting region PXA-B.

The base substrate BL may be disposed on the color filter layer CFL. The base substrate BL may be a member providing a base surface on which the color filter layer CFL and the light control layer CCL are disposed. The base substrate BL may be a glass substrate, a metal substrate, a plastic substrate, or the like. However, aspects of the present disclosure are not limited thereto, and the base substrate BL may be an inorganic layer, an organic layer, or a composite material layer. In addition, unlike what is shown, the base substrate BL may not be provided in an aspect.

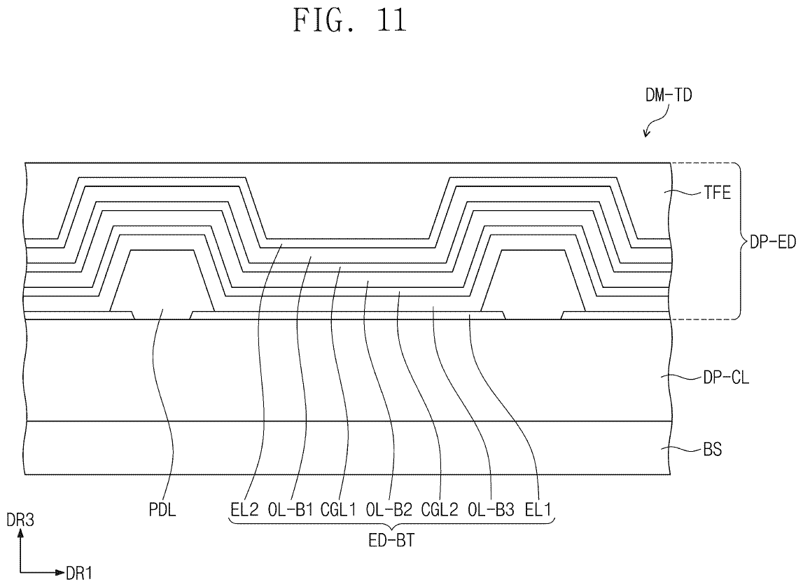

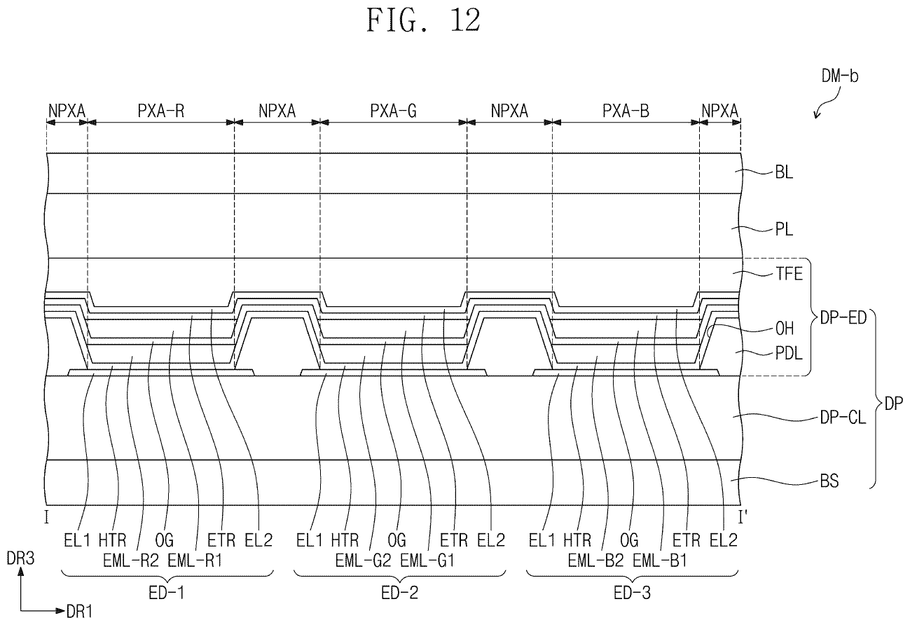

FIG. 11 is a cross-sectional view showing a portion of a display module according to an aspect; In a display module DM-TD according to an aspect, a light emitting element ED-BT may include a plurality of light emitting structures OL-B1, OL-B2, and OL-B3. At least one of the plurality of light emitting structures OL-B1, OL-B2, and OL-B3 may include the amine compound of an aspect. Accordingly, the light emitting element ED-BT may exhibit long lifespan characteristics. In addition, the light emitting element ED-BT according to an aspect may emit blue light and exhibit long lifespan characteristics.

The light emitting element ED-BT may include the first electrode EL1 and the second electrode EL2 facing each other, and the plurality of light emitting structures OL-B1, OL-B2, and OL-B3 provided by being sequentially stacked in a thickness direction between the first electrode EL1 and the second electrode EL2. The light emitting structures OL-B1, OL-B2, and OL-B3 each may include the emission layer EML (FIG. 5), a hole transport region HTR and an electron transport region ETR disposed with the emission layer EML (FIG. 8) therebetween. That is, the light emitting element ED-BT included in the display module DM-TD according to an aspect may be a light emitting element having a tandem structure including a plurality of emission layers.

In an aspect shown in FIG. 11, light emitted from each of the light emitting structures OL-B1, OL-B2, and OL-B3 may all be blue light. However, aspects of the present disclosure are not limited thereto, and wavelength ranges of light emitted from each of the light emitting structures OL-B1, OL-B2, and OL-B3 may be different from each other. For example, the light emitting element ED-BT including the plurality of light emitting structures OL-B1, OL-B2, and OL-B3 emitting light in different wavelength ranges may emit white light.

Charge generation layers CGL1 and CGL2 may be disposed between neighboring light emitting structures OL-B1, OL-B2, and OL-B3. In an aspect shown in FIG. 11, the charge generation layers CGL1 and CGL2 may include a first charge generation layer CGL1 disposed between the first light emitting structure OL-B1 and the second light emitting structure OL-B2, and a second charge generation layer CGL2 disposed between the second light emitting structure OL-B2 and the third light emitting structure OL-B3. The first and second charge generation layers CGL1 and CGL2 may each independently include a p-type charge generation layer and/or an n-type charge generation layer.

Referring to FIG. 12, a display module DM-b according to an aspect may include light emitting elements ED-1, ED-2, and ED-3 in which two emission layers are stacked. At least one of the light emitting elements ED-1, ED-2, and ED-3 may include the amine compound according to an aspect in the hole transport region HTR. Accordingly, the light emitting elements ED-1, ED-2, and ED-3 may exhibit long lifespan characteristics. In addition, the light emitting element ED-3 according to an aspect may exhibit long lifespan characteristics in a blue light emission region.

Compared to the display module DM according to an aspect shown in FIG. 4, the difference is that in an aspect shown in FIG. 12, the first to third light emitting elements ED-1, ED-2, and ED-3 each include two emission layers stacked in a thickness direction. In each of the first to third light emitting elements ED-1, ED-2, and ED-3, the two emission layers may emit light in the same wavelength range.

The first light emitting element ED-1 may include a first red emission layer EML-R1 and a second red emission layer EML-R2. The second light emitting element ED-2 may include a first green emission layer EML-G1 and a second green emission layer EML-G2. In addition, the third light emitting element ED-3 may include a first blue emission layer EML-B1 and a second blue emission layer EML-B2. A light emitting auxiliary portion OG may be disposed between the first red emission layer EML-R1 and the second red emission layer EML-R2, between the first green emission layer EML-G1 and the second green emission layer EML-G2, and between the first blue emission layer EML-B1 and the second blue emission layer EML-B2.

The light emitting auxiliary portion OG may include a single layer or multiple layers. The light emitting auxiliary portion OG may include a charge generation layer. To be more specific, the light emitting auxiliary portion OG may include an electron transport region, a charge generation layer, and a hole transport region that are sequentially stacked. The light emitting auxiliary portion OG may be provided as a common layer throughout the first to third light emitting elements ED-1, ED-2, and ED-3. However, aspects of the present disclosure are not limited thereto, and the light emitting auxiliary portion OG may be provided to be patterned inside the openings OH defined in the pixel defining films PDL.

The first red emission layer EML-R1, the first green emission layer EML-G1, and the first blue emission layer EML-B1 may be disposed between the light emitting auxiliary portion OG and the electron transport region ETR. The second red emission layer EML-R2, the second green emission layer EML-G2, and the second blue emission layer EML-B2 may be disposed between the hole transport region HTR and the light emitting auxiliary portion OG.

That is, the light emitting element ED-1 may include the first electrode EL1, the hole transport region HTR, the second red emission layer EML-R2, the emission auxiliary portion OG, the first red emission layer EML-R1, the electron transport region ETR, and the second electrode EL2, which are sequentially stacked. The second light emitting element ED-2 may include the first electrode EL1, the hole transport region HTR, the second green emission layer EML-G2, the emission auxiliary portion OG, the first green emission layer EML-G1, the electron transport region ETR, and the second electrode EL2, which are sequentially stacked. The third light emitting element ED-3 may include the first electrode EL1, the hole transport region HTR, the second blue emission layer EML-B2, the emission auxiliary portion OG, the first blue emission layer EML-B1, the electron transport region ETR, and the second electrode EL2, which are sequentially stacked.

An optical auxiliary layer PL may be disposed on the display element layer DP-ED. The optical auxiliary layer PL may include a polarizing layer. The optical auxiliary layer PL may be disposed on the display panel DP to control reflected light in the display panel DP due to external light. Unlike what is shown, the optical auxiliary layer PL may be omitted in the display device according to an aspect.

Unlike FIGS. 11 and 12, a display module DM-c according to an aspect shown in FIG. 13 is shown to include four light emitting structures OL-B3, OL-B2, OL-B1, and OL-C1. A light emitting element ED-CT may include the first electrode EL1 and the second electrode EL2 facing each other, and the first to fourth light emitting structures L-B1, OL-B2, OL-B3, and OL-C1 sequentially stacked in a thickness direction between the first electrode EL1 and the second electrode EL2. At least one of the first to fourth light emitting structures OL-B1, OL-B2, OL-B3, and OL-C1 may include the amine compound according to an aspect. Accordingly, the light emitting element ED-CT may exhibit long lifespan characteristics. In addition, the light emitting element ED-CT according to an aspect may exhibit improved lifespan characteristics in a blue light emission region.

Charge generation layers CGL3, CGL2, and CGL1 may be disposed between the first to fourth light emitting structures OL-B1, OL-B2, OL-B3, and OL-C1. Among the four light emitting structures, the first to third light emitting structures OL-B1, OL-B2, and OL-B3 may emit blue light, and the fourth light emitting structure OL-C1 may emit green light. However, aspects of the present disclosure are not limited thereto, and the first to fourth light emitting structures OL-B1, OL-B2, OL-B3, and OL-C1 may emit light having different wavelength ranges. The charge generation layers CGL3, CGL2 and CGL1 disposed between the neighboring light emitting structures OL-B1, OL-B2, OL-B3, and OL-C1 may include a p-type charge generation layer and/or an n-type charge generation layer.

In an aspect, an electronic device may include a display device including a plurality of light emitting elements, and a control portion configured to control the display device. In the electronic device according to an aspect, at least one of the plurality of light emitting elements may include the amine compound according to an aspect in a hole transport region. For example, at least one of the plurality of light emitting elements included in the electronic device according to an aspect may include the amine compound according to an aspect in a hole transport region.

The electronic device according to an aspect may be a device activated in response to electrical signals. The electronic device according to an aspect may include display devices including the display modules according to an aspect described with reference to FIG. 4, and FIGS. 10 to 13. For example, the electronic device may include large-sized display devices such as televisions, monitors, or outdoor billboards, as well as small- and medium-sized display devices such as personal computers, laptop computers, personal digital terminals, in-vehicle display devices, game consoles, portable electronic devices, or cameras.

The electronic device according to an aspect includes a display module including the amine compound according to an aspect, and may thus exhibit improved reliability and long lifespan characteristics. The electronic device according to an aspect may have improved display lifespan and exhibit excellent display quality.

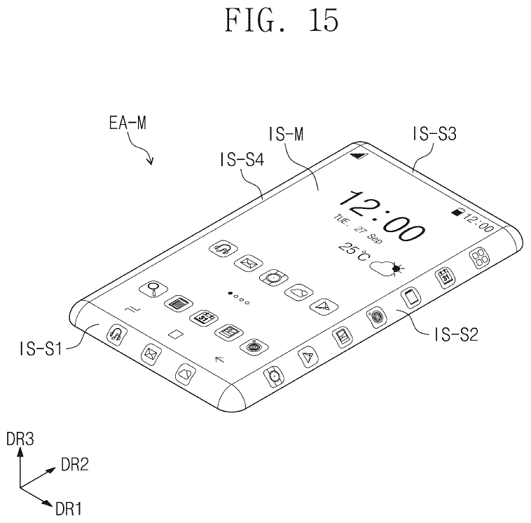

FIG. 14 shows a tablet terminal as an example of the electronic device EA. The electronic device EA according to an aspect may include a display module DM according to an aspect. For example, electronic modules, a camera module, a power module, or the like mounted on a main board together with the display module DM are disposed in a bracket/housing HAU to form a tablet terminal.

The electronic device EA according to an aspect shown in FIG. 14 may include the display modules according to an aspect described with reference to FIG. 4 and FIGS. 10 to 13.

The electronic device EA including the display module DM provided with a flat display surface is shown in an aspect, but aspects of the present disclosure are not limited thereto. The electronic device EA may include a curved display surface or a three-dimensional display surface. For example, the three-dimensional display surface may include a plurality of display regions indicating different directions, and may also include a bent display surface. The electronic device EA according to the present aspect may be a flexible electronic device. The flexible electronic device may be a foldable electronic device.

As shown in FIG. 14, the display surface EA-IS includes an active region AA on which images are displayed and a bezel region NAA adjacent to the active region AA. The bezel region NAA is a region on which images are not displayed. In FIG. 14, icon images are shown as an example of images. The active region AA may be referred to as a display region of the display module DM, and the bezel region NAA may be referred to as a non-display region of the display module DM.

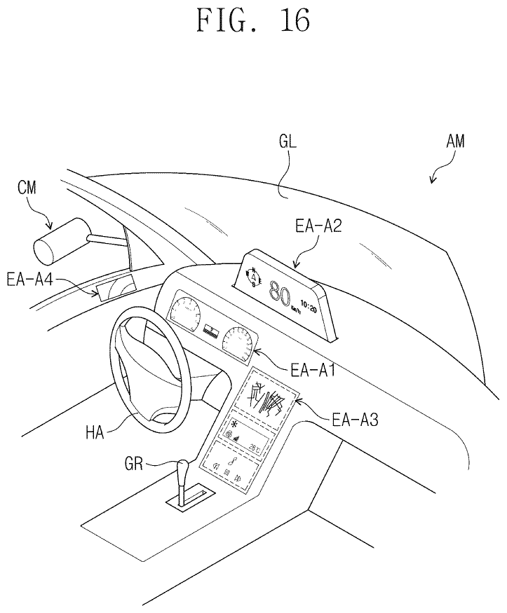

FIG. 15 shows a portable terminal as an example of an electronic device EA-M. Referring to FIG. 15, the electronic device EA-M according to an aspect may include a plurality of display surfaces. The electronic device EA-M according to an aspect may include display surfaces IS-M, IS-S1, IS-S2, IS-S3, and IS-S4 with different primary display directions.

In an aspect, the electronic device EA-M may be a stereoscopic display device including an upper display surface IS-M and a plurality of side display surfaces IS-S1, IS-S2, IS-S3, and IS-S4. Each of the side display surfaces IS-S1, IS-S2, IS-S3, and IS-S4 may be a display surface extending from one side of the upper display surface IS-M. In an aspect, the electronic device EA-M may include a main display surface that primarily displays images in one direction and a plurality of sub-display surfaces that display images in a direction different from the main display surface. In an aspect of the electronic device EA-M shown in FIG. 15, the primary display surface may be the upper display surface IS-M, and the sub-display surfaces may be the side display surfaces IS-S1, IS-S2, IS-S3, and IS-S4.

The side display surfaces IS-S1, IS-S2, IS-S3, and IS-S4 may have display surfaces that are not parallel to the upper display surface IS-M. Meanwhile, the plurality of side display surfaces IS-S1, IS-S2, IS-S3, and IS-S4 may be display regions that are each bent and extended from one side of the upper display surface IS-M. For example, the plurality of side display surfaces IS-S1, IS-S2, IS-S3, and IS-S4 may be bending display regions.

The electronic device EA-M according to an aspect shown in FIG. 15 may include the display modules according to an aspect described with reference to FIG. 4 and FIGS. 10 to 13.

FIG. 16 is a view showing a vehicle AM in which first to fourth electronic devices EA-1, EA-2, EA-3, and EA-4 are disposed. At least one of the first to fourth electronic devices EA-1, EA-2, EA-3, or EA-4 may include the display modules according to an aspect described with reference to FIG. 4 and FIGS. 10 to 13.

FIG. 16 shows a car as the vehicle AM, but this is presented as an example, and the first to fourth electronic devices EA-1, EA-2, EA-3, and EA-4 may be disposed on other means of transportation, such as bicycles, motorcycles, trains, ships, and airplanes.

At least one of the first to fourth electronic devices EA-1, EA-2, EA-3, or EA-4 may include the light emitting element ED according to an aspect described with reference to FIGS. 5 to 9. At least one of the first to fourth electronic devices EA-1, EA-2, EA-3, or EA-4 may include the amine compound according to an aspect. Accordingly, the first to fourth electronic devices EA-1, EA-2, EA-3, and EA-4 including the amine compound according to an aspect may exhibit improved display lifespan. In addition, the first to fourth electronic devices EA-1, EA-2, EA-3, and EA-4 including the amine compound according to an aspect may exhibit excellent display quality and improved reliability.

Referring to FIG. 16, the vehicle AM may include a wheel HA and a gear GR for operation control, and include a front window GL disposed to face a driver.

The first electronic device EA-1 may be a digital cluster displaying first information of the vehicle AM. The first information may include a first scale indicating driving speed of the vehicle AM, a second scale indicating engine revolutions (revolutions per minute (RPM)), and an image indicating fuel gauge, or the like. The first scale and the second scale may be displayed as digital images. In an aspect shown in FIG. 16, the first display device EA-1 may be disposed in a first region overlapping the wheel HA. However, aspects of the present disclosure are not limited thereto, and the first electronic device EA-1 may be disposed across the entire dashboard, or may be disposed separately on a portion facing a driver seat and a portion facing a passenger seat.

The second display device EA-2 may be disposed in a second region between a driver seat and the front window GL. For example, the second electronic device EA-2 may be a head up display HUD displaying second information of the vehicle AM. The second electronic device EA-2 may be optically transparent. The second information includes digital numbers indicating driving speed of the vehicle AM and may further include information such as current time. Unlike what is shown, the second information of the second electronic device EA-2 may be projected and displayed on the front window GL. The display surface of the second electronic device EA-2 may face a driver seat. Alternatively, the second electronic device EA-2 may also provide images toward the front window GL.

The third electronic device EA-3 may be disposed in a third region adjacent to the gear GR. For example, the third display device EA-3 may be a center information display CID for a vehicle, which is disposed between a driver seat and a passenger seat, and displays third information. The passenger seat may be a seat spaced apart from the driver seat with the gear GR therebetween. The third information may include information about road conditions (e.g., navigation information), music or radio play, dynamic video play, temperature inside the vehicle AM, or the like.

The fourth display device EA-4 may be disposed in a fourth region spaced apart from the wheel HA and the gear GR and adjacent to a side of the vehicle AM. For example, the fourth electronic device EA-4 may be a digital side mirror displaying fourth information. The fourth electronic device EA-4 may display images of conditions outside the vehicle AM, which are taken by the camera module CM disposed outside the vehicle AM. The fourth information may include images of conditions outside the vehicle AM.

The first to fourth information described above are presented as an example, and the first to fourth electronic devices EA-1, EA-2, EA-3, and EA-4 may further display information about inside or outside a vehicle. The first to fourth information may include different information. However, aspects of the present disclosure are not limited thereto, and some of the first to fourth information may include the same information.

FIGS. 14 to 16 show an example of an electronic device or an example including an electronic device, and a display module including a light emitting element including an amine compound according to an aspect may be adopted for other electronic devices without departing from the present disclosure.

Hereinafter, with reference to Examples and Comparative Examples, an amine compound according to an aspect of the present disclosure and a light emitting element according to an aspect will be specifically described. In addition, Examples below are shown only for the understanding of the present disclosure, and the scope of the present disclosure is not limited thereto.

### EXAMPLES

### 1. Synthesis of Amine Compounds of Examples

A process of synthesizing amine compounds according to an aspect of the present disclosure will be described in detail by presenting a process of synthesizing amine compounds 2, 12, 13, 23, 24, 29, 34, 42, 62, 66, 133, 197, and 400 as an example. In addition, a process of synthesizing amine compounds, which will be described hereinafter, is provided as an example, and thus a process of synthesizing compounds according to an aspect of the present disclosure is not limited to Examples below.

In the synthetic method of the amine compound, which will be described below, a molecular weight of the compound was measured by FAB-MS using JMS-700V (JEOL Ltd.).

### (1) Synthesis of Amine Compound 2

Amine compound 8 according to an aspect may be synthesized by, for example, processes of Reaction Formula 1 below.

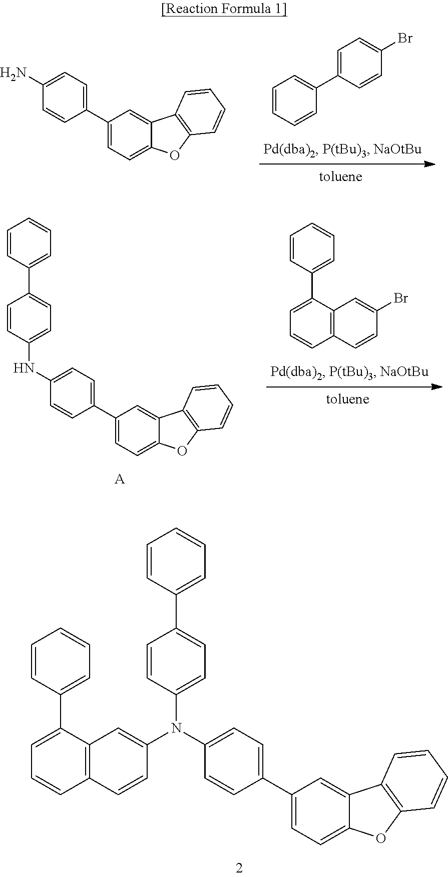

### <Synthesis of Compound 2>

In an argon (Ar) atmosphere, 4-(dibenzofuran-2-yl) aniline (20.0 g), 4-bromobiphenyl (17.3 g), bis(dibenzylideneacetone) palladium (0) (Pd(dba)₂, 0.4 g), and sodium tert-butoxide (NaOtBu, 10.7 g) were added to a 1 L 3-necked flask, and the mixture was dissolved in toluene (400 mL), and tri-tert-butylphosphine (P(tBu)₃, 2.0 M in toluene, 0.7 mL) was added and stirred at room temperature for 4 hours and then stirred at 100° C. for 4 hours. Water was added thereto for extraction using CH₂Cl₂ and an organic layer was collected and dried using MgSO₄, and then a solvent was distilled off under reduced pressure. The obtained crude product was purified through silica gel column chromatography to obtain 25.7 g of Compound A (yield: 84%). A molecular weight of Compound A as determined through FAB-MS was 411.

In an argon (Ar) atmosphere, Compound A (5.0 g), 7-bromo-1-phenylnaphthalene (3.5 g), Pd(dba)₂ (0.07 g), and NaOtBu (1.8 g) were added to a 300 mL 3-necked flask, and the mixture was dissolved in toluene (100 mL), and P(tBu)₃ (2.0 M in toluene, 0.1 mL) was added, and heated and refluxed for 4 hours. Water was added thereto for extraction using CH₂Cl₂ and an organic layer was collected and dried using MgSO₄, and then a solvent was distilled off under reduced pressure. The obtained crude product was purified through silica gel column chromatography to obtain 6.0 g of Compound 2 (yield: 81%). A molecular weight of Compound 2 as determined through FAB-MS was 613.

### (2) Synthesis of Compound 12

Amine Compound 12 according to an aspect may be synthesized by, for example, processes of Reaction Formula 2 below.

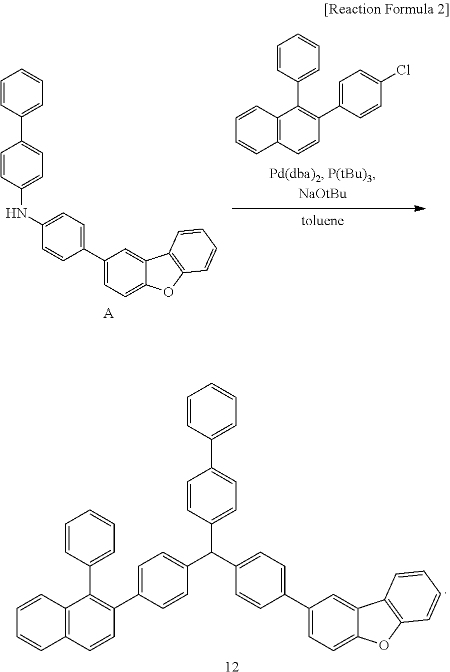

In the same manner as the synthesis of Compound 2 described above, 6.8 g of Compound 12 (yield: 81%) was obtained from Intermediate Compound A (5.0 g) and 2-(4-chlorophenyl)-1-phenylnaphthalene (3.9 g). A molecular weight of Compound 12 as determined through FAB-MS was 689.

### (3) Synthesis of Amine Compound 13

Amine compound 13 according to an aspect may be synthesized by, for example, processes of Reaction Formula 3 below.

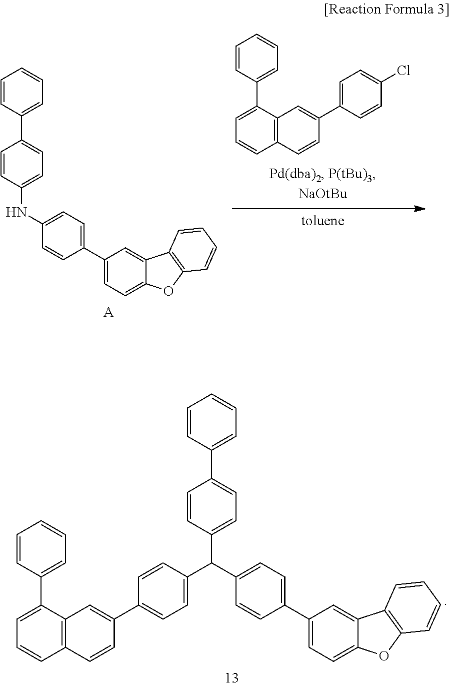

In the same manner as the synthesis of Compound 2 described above, 6.4 g of Compound 13 (yield: 76%) was obtained from Intermediate Compound A (5.0 g) and 7-(4-chlorophenyl)-1-phenylnaphthalene (3.9 g). A molecular weight of Compound 13 as determined through FAB-MS was 689.

### (4) Synthesis of Amine Compound 23

Amine compound 23 according to an aspect may be synthesized by, for example, processes of Reaction Formula 4 below.

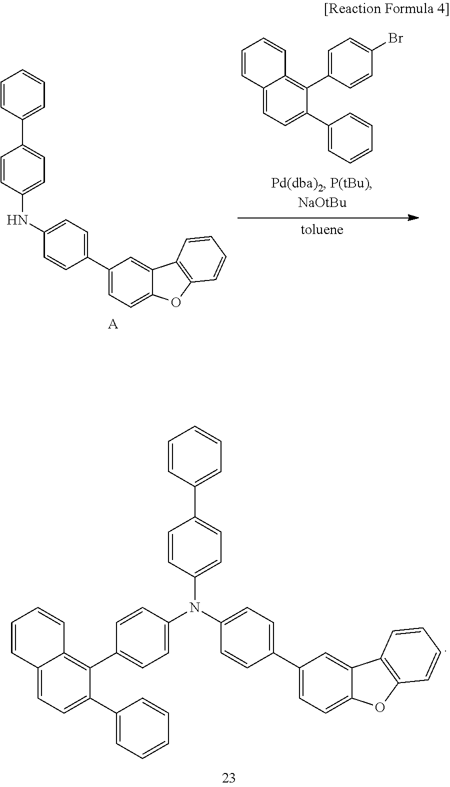

In the same manner as the synthesis of Compound 2 described above, 6.0 g of Compound 23 (yield: 71%) was obtained from Intermediate Compound A (5.0 g) and 1-(4-bromophenyl)-2-phenylnaphthalene (3.9 g). A molecular weight of Compound 23 as determined through FAB-MS was 689.

### (5) Synthesis of Amine Compound 24

Amine compound 24 according to an aspect may be synthesized by, for example, processes of Reaction Formula 5 below.

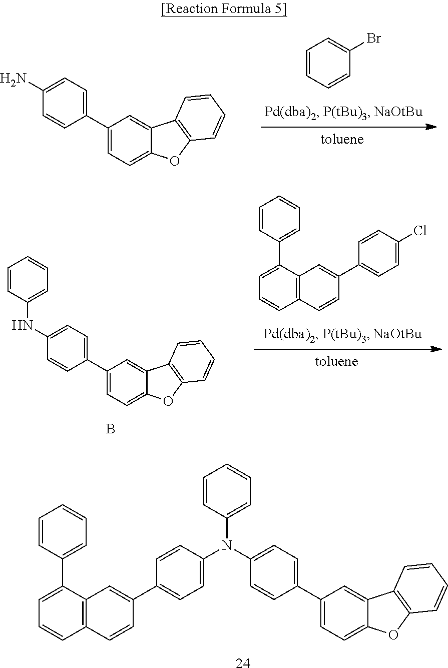

In the same manner as the synthesis of Compound A described above, 9.1 g of Compound B (yield: 70%) was obtained from 4-(dibenzofuran-2-yl) aniline (10.0 g) and bromobenzene (6.0 g). A molecular weight of Compound B as determined through FAB-MS was 335.

In the same manner as the synthesis of Compound 2 described above, 7.8 g of Compound 24 (yield: 85%) was obtained from Intermediate Compound B (5.0 g) and 7-(4-chlorophenyl)-1-phenylnaphthalene (4.6 g). A molecular weight of Compound 24 as determined through FAB-MS was 613.

### (6) Synthesis of Amine Compound 29

Amine compound 29 according to an aspect may be synthesized by, for example, processes of Reaction Formula 6 below.

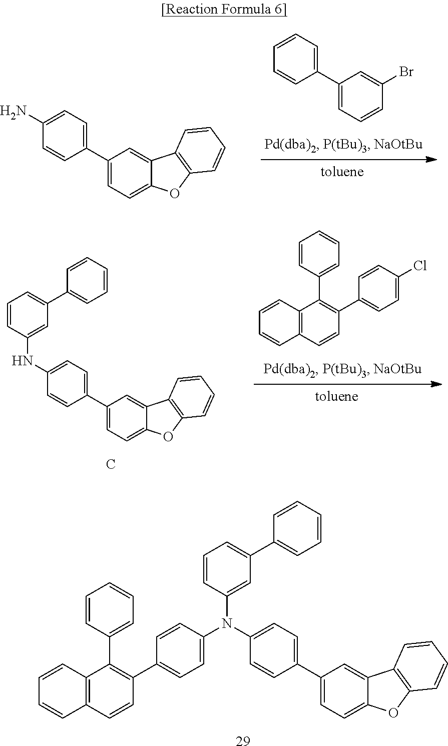

In the same manner as the synthesis of Compound A described above, 10.3 g of Compound C (yield: 65%) was obtained from 4-(dibenzofuran-2-yl) aniline (10.0 g) and 3-bromobiphenyl (8.9 g). A molecular weight of Compound C as determined through FAB-MS was 411.

In the same manner as the synthesis of Compound 2 described above, 6.7 g of Compound 29 (yield: 80%) was obtained from Intermediate Compound C (5.0 g) and 2-(4-chlorophenyl)-1-phenylnaphthalene (3.9 g). A molecular weight of Compound 29 as determined through FAB-MS was 689.

### (7) Synthesis of Amine Compound 34

Amine compound 34 according to an aspect may be synthesized by, for example, processes of Reaction Formula 7 below.

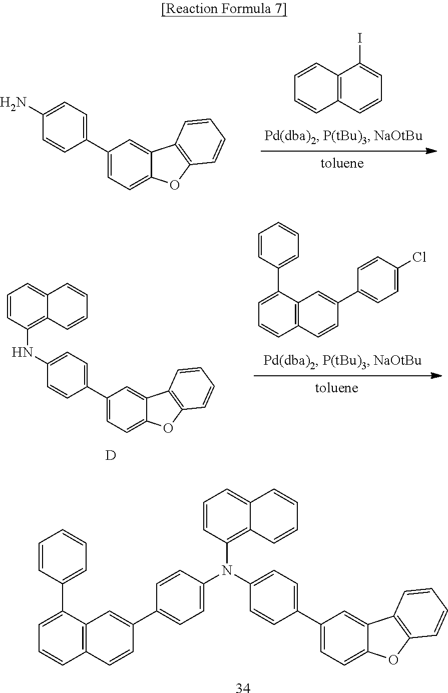

In the same manner as the synthesis of Compound A described above, 8.3 g of Compound D (yield: 56%) was obtained from 4-(dibenzofuran-2-yl) aniline (10.0 g) and 1-iodonaphthalene (9.7 g). A molecular weight of Compound D as determined through FAB-MS was 385.

In the same manner as the synthesis of Compound 2 described above, 7.1 g of Compound 34 (yield: 82%) was obtained from Intermediate Compound D (5.0 g) and 7-(4-chlorophenyl)-1-phenylnaphthalene (4.1 g). A molecular weight of Compound 34 as determined through FAB-MS was 663.

### (8) Synthesis of Amine Compound 42

Amine compound 42 according to an aspect may be synthesized by, for example, processes of Reaction Formula 8 below.

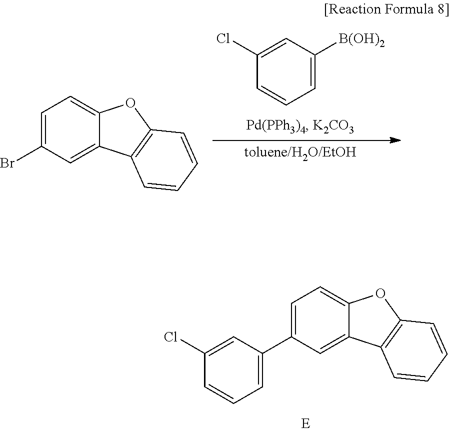 [SMILES: Brc1ccc2oc3ccccc3c2c1 ; OB(O)c1cccc(Cl)c1 ; Clc1cccc(-c2ccc3oc4ccccc4c3c2)c1]

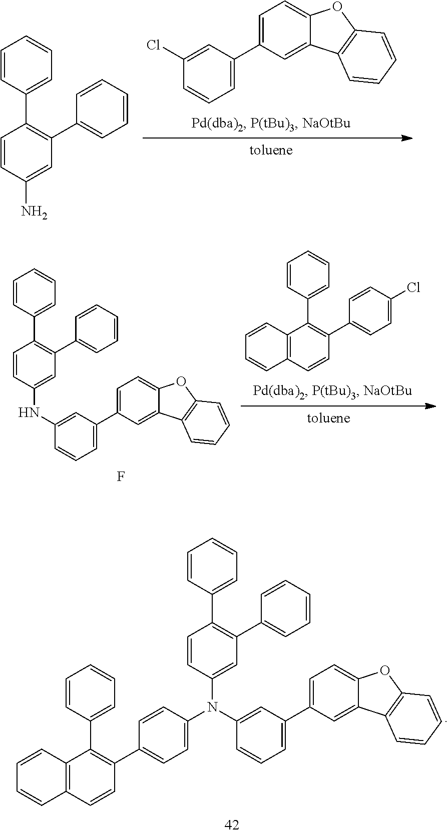

In an argon (Ar) atmosphere, 2-bromodibenzofuran (10.0 g), (3-chlorophenyl) boronic acid (6.3 g), tetrakis(triphenylphosphine) palladium (0) (Pd(PPh₃)₄, 2.3 g), and potassium carbonate (K₂CO₃, 11.2 g) were added to a 1 L 3-necked flask, and the mixture was dissolved in a mixed solvent of toluene, water, and ethanol (10:2:1, 300 mL), and heated and stirred at 80° C. for 16 hours. Water was added thereto for extraction using CH₂Cl₂ and an organic layer was collected and dried using MgSO₄, and then a solvent was distilled off under reduced pressure. The obtained crude product was purified through silica gel column chromatography and recrystallization to obtain 9.0 g of Compound E (yield: 80%). A molecular weight of Compound E as determined through FAB-MS was 278.

In the same manner as the synthesis of Compound A described above, 8.7 g of Compound F (yield: 55%) was obtained from [1,1′:2′,1″-terphenyl]-4′-amine (8.0 g) and Compound E (9.0 g). A molecular weight of Compound F as determined through FAB-MS was 487.

In the same manner as the synthesis of Compound 2 described above, 6.0 g of Compound 42 (yield: 77%) was obtained from Compound F (5.0 g) and 2-(4-chlorophenyl)-1-phenylnaphthalene (3.3 g). A molecular weight of Compound 42 as determined through FAB-MS was 765.

### (9) Synthesis of Amine Compound 62

Amine compound 62 according to an aspect may be synthesized by, for example, processes of Reaction Formula 9 below.

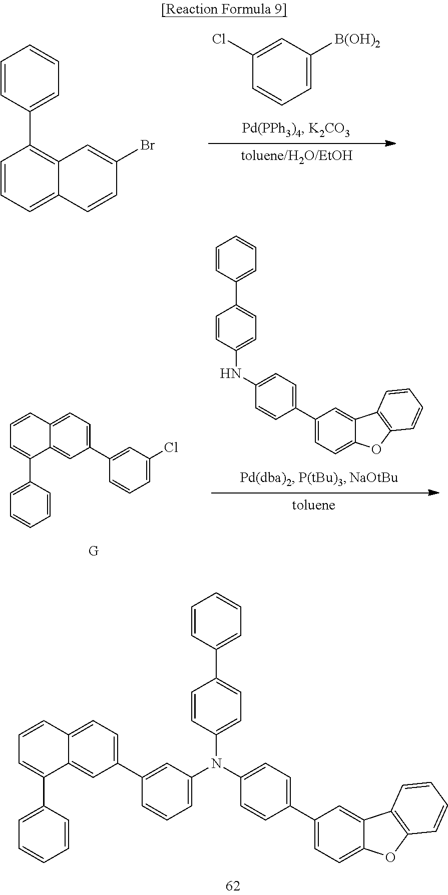 [SMILES: Brc1ccc2cccc(-c3ccccc3)c2c1 ; OB(O)c1cccc(Cl)c1 ; Clc1cccc(-c2ccc3cccc(-c4ccccc4)c3c2)c1 ; c1ccc(-c2ccc(Nc3ccc(-c4ccc5oc6ccccc6c5c4)cc3)cc2)cc1 ; c1ccc(-c2ccc(N(c3ccc(-c4ccc5oc6ccccc6c5c4)cc3)c3cccc(-c4ccc5cccc(-c6ccccc6)c5c4)c3)cc2)cc1]

In the same manner as the synthesis of Compound E described above, 7.4 g of Compound G (yield: 67%) was obtained from 7-bromo-1-phenylnaphthalene (10.0 g) and (3-clorophenyl) boronic acid (5.5 g). A molecular weight of Compound G as determined through FAB-MS was 314.

In the same manner as the synthesis of Compound 2 described above, 6.8 g of Compound 62 (yield: 79%) was obtained from Intermediate Compound G (3.9 g) and Compound A (5.0 g). A molecular weight of Compound 62 as determined through FAB-MS was 689.

### (10) Synthesis of Amine Compound 66

Amine compound 66 according to an aspect may be synthesized by, for example, processes of Reaction Formula 10 below.

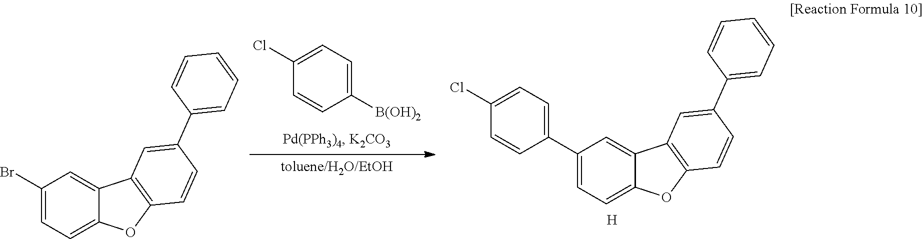

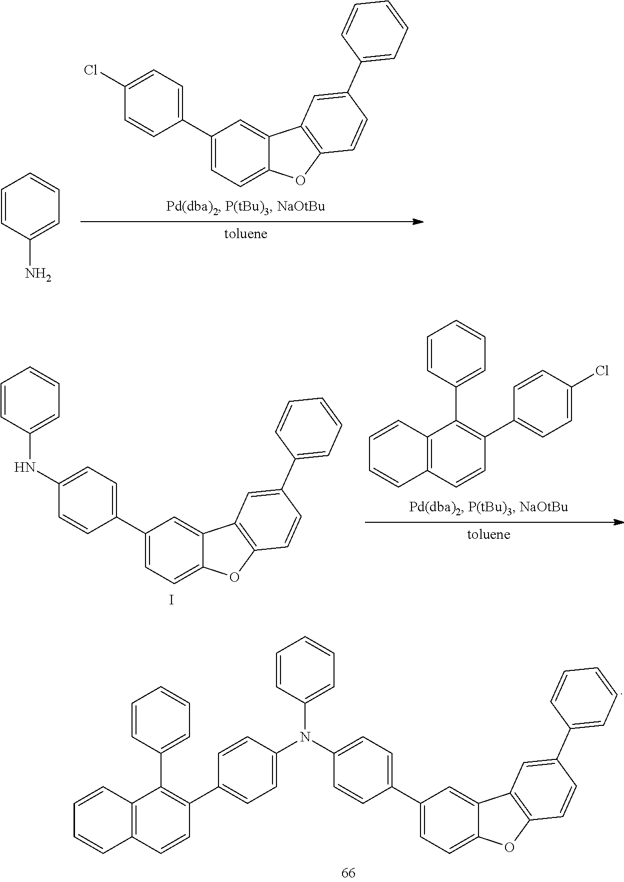

In the same manner as the synthesis of Compound E described above, 8.8 g of Compound H (yield: 80%) was obtained from 2-bromo-8-phenyldibenzofuran (10.0 g) and (4-clorophenyl) boronic acid (4.8 g). A molecular weight of Compound H as determined through FAB-MS was 354.

In the same manner as the synthesis of Compound A described above, 5.7 g of Compound I (yield: 64%) was obtained from Aniline (2.0 g) and Compound H (7.6 g). A molecular weight of Compound I as determined through FAB-MS was 411.

In the same manner as the synthesis of Compound 2 described above, 5.9 g of Compound 66 (yield: 70%) was obtained from Intermediate Compound I (5.0 g) and 2-(4-chlorophenyl)-1-phenylnaphthalene (3.8 g). A molecular weight of Compound 66 as determined through FAB-MS was 689.

### (11) Synthesis of Amine Compound 133

Amine compound 133 according to an aspect may be synthesized by, for example, processes of Reaction Formula 11 below.

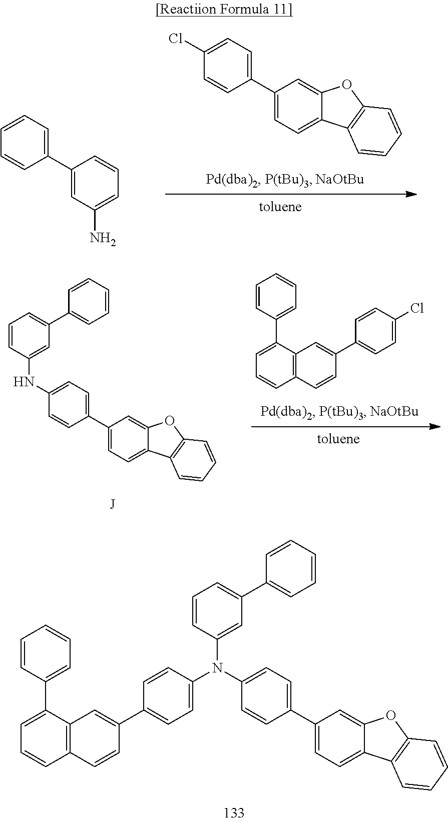 [SMILES: Nc1cccc(-c2ccccc2)c1 ; Clc1ccc(-c2ccc3c(c2)oc2ccccc23)cc1 ; c1ccc(-c2cccc(Nc3ccc(-c4ccc5c(c4)oc4ccccc45)cc3)c2)cc1 ; Clc1ccc(-c2ccc3cccc(-c4ccccc4)c3c2)cc1 ; c1ccc(-c2cccc(N(c3ccc(-c4ccc5c(c4)oc4ccccc45)cc3)c3ccc(-c4ccc5cccc(-c6ccccc6)c5c4)cc3)c2)cc1]

In the same manner as the synthesis of Compound A described above, 17.0 g of Compound J (yield: 70%) was obtained from 3-aminobiphenyl (10.0 g) and 3-(4-chlorophenyl)dibenzofuran (16.4 g). A molecular weight of Compound J as determined through FAB-MS was 411.

In the same manner as the synthesis of Compound 2 described above, 5.4 g of Compound 133 (yield: 65%) was obtained from Intermediate Compound J (5.0 g) and 7-(4-chlorophenyl)-1-phenylnaphthalene (3.9 g). A molecular weight of Compound 133 as determined through FAB-MS was 689.

### (12) Synthesis of Amine Compound 197

Amine compound 197 according to an aspect may be synthesized by, for example, processes of Reaction Formula 12 below.

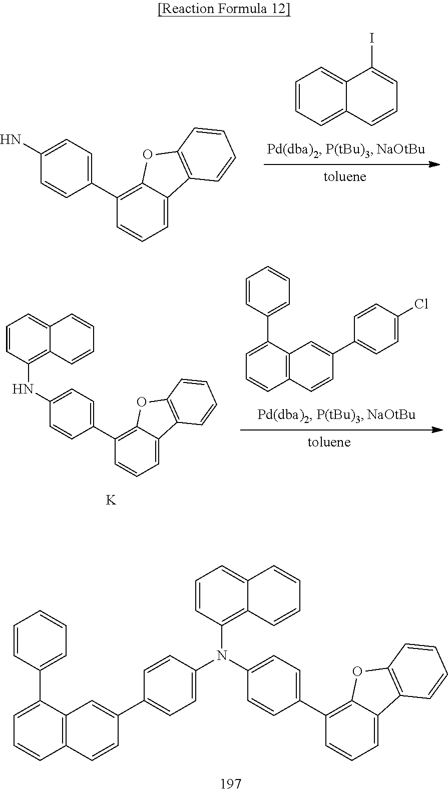

In the same manner as the synthesis of Compound A described above, 7.1 g of Compound K (yield: 48%) was obtained from 4-(dibenzofuran-4-yl) aniline (10.0 g) and 1-iodonaphthalene (9.7 g). A molecular weight of Compound K as determined through FAB-MS was 385.

In the same manner as the synthesis of Compound 2 described above, 7.0 g of Compound 197 (yield: 81%) was obtained from Intermediate Compound K (5.0 g) and 2-(4-chlorophenyl)-1-phenylnaphthalene (4.1 g). A molecular weight of Compound 197 as determined through FAB-MS was 663.

### (13) Synthesis of Amine Compound 400

Amine compound 400 according to an aspect may be synthesized by, for example, processes of Reaction Formula 13 below.

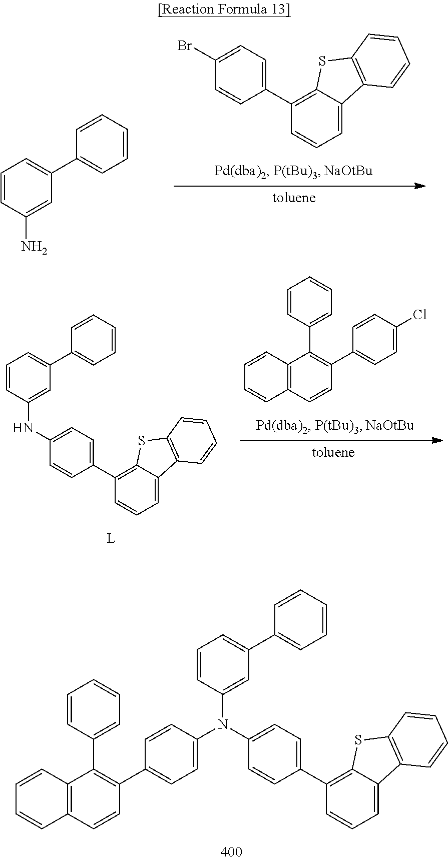 [SMILES: Nc1cccc(-c2ccccc2)c1 ; Brc1ccc(-c2cccc3c2sc2ccccc23)cc1 ; c1ccc(-c2cccc(Nc3ccc(-c4cccc5c4sc4ccccc45)cc3)c2)cc1 ; Clc1ccc(-c2ccc3ccccc3c2-c2ccccc2)cc1 ; c1ccc(-c2cccc(N(c3ccc(-c4ccc5ccccc5c4-c4ccccc4)cc3)c3ccc(-c4cccc5c4sc4ccccc45)cc3)c2)cc1]

In the same manner as the synthesis of Compound A described above, 8.5 g of Compound L (yield: 67%) was obtained from 3-aminobiphenyl (5.0 g) and 4-(4-bromophenyl)dibenzothiophene (10.0 g). A molecular weight of Compound L as determined through FAB-MS was 427.

In the same manner as the synthesis of Compound 2 described above, 6.0 g of Compound 400 (yield: 73%) was obtained from Intermediate Compound L (5.0 g) and 2-(4-chlorophenyl)-1-phenylnaphthalene (3.7 g). A molecular weight of Compound 400 as determined through FAB-MS was 705.

### (14) Synthesis of Amine Compound 3

Amine compound 3 according to an aspect may be synthesized by, for example, processes of Reaction Formula 14 below.

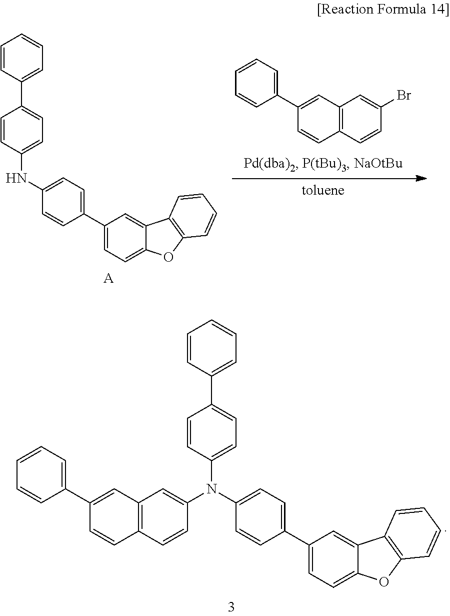

In the same manner as the synthesis of Compound 2 described above, 6.0 g of Compound 3 (yield: 80%) was obtained from Intermediate Compound A (5.0 g) and 2-bromo-7-phenylnaphthalene (3.5 g). A molecular weight of Compound 3 as determined through FAB-MS was 613.

### (15) Synthesis of Amine Compound 64

Amine compound 64 according to an aspect may be synthesized by, for example, processes of Reaction Formula 15 below.

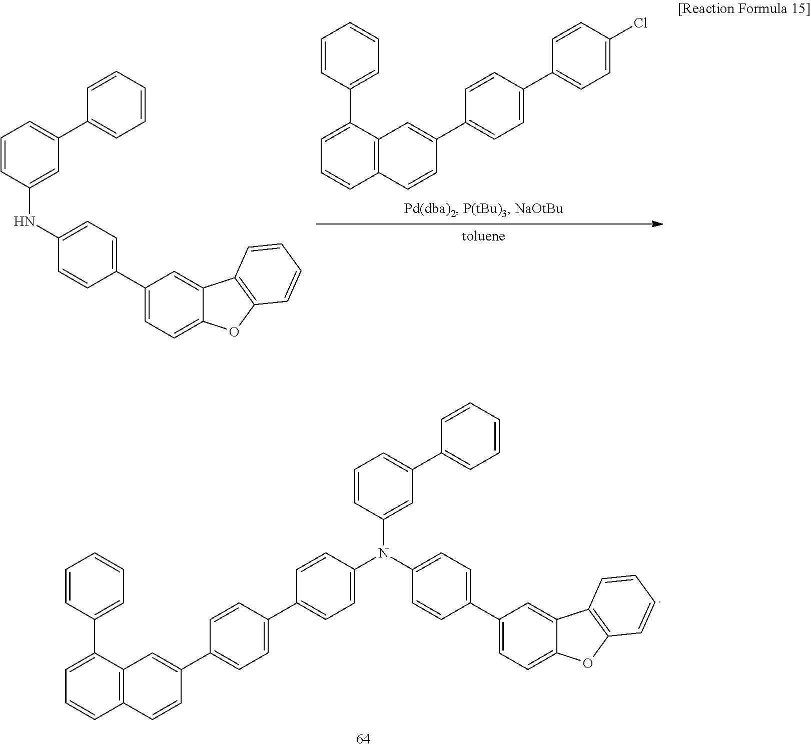 [SMILES: c1ccc(-c2cccc(Nc3ccc(-c4ccc5oc6ccccc6c5c4)cc3)c2)cc1 ; Clc1ccc(-c2ccc(-c3ccc4cccc(-c5ccccc5)c4c3)cc2)cc1 ; c1ccc(-c2cccc(N(c3ccc(-c4ccc(-c5ccc6cccc(-c7ccccc7)c6c5)cc4)cc3)c3ccc(-c4ccc5oc6ccccc6c5c4)cc3)c2)cc1]

In the same manner as the synthesis of Compound 2 described above, 6.0 g of Compound 64 (yield: 65%) was obtained from Intermediate Compound C (5.0 g) and 7-(4′-chloro-[1,1′-biphenyl]-4-yl)-1-phenylnaphthalene (4.8 g). A molecular weight of Compound 64 as determined through FAB-MS was 765.

### (16) Synthesis of Amine Compound 65

Amine compound 65 according to an aspect may be synthesized by, for example, processes of Reaction Formula 16 below.

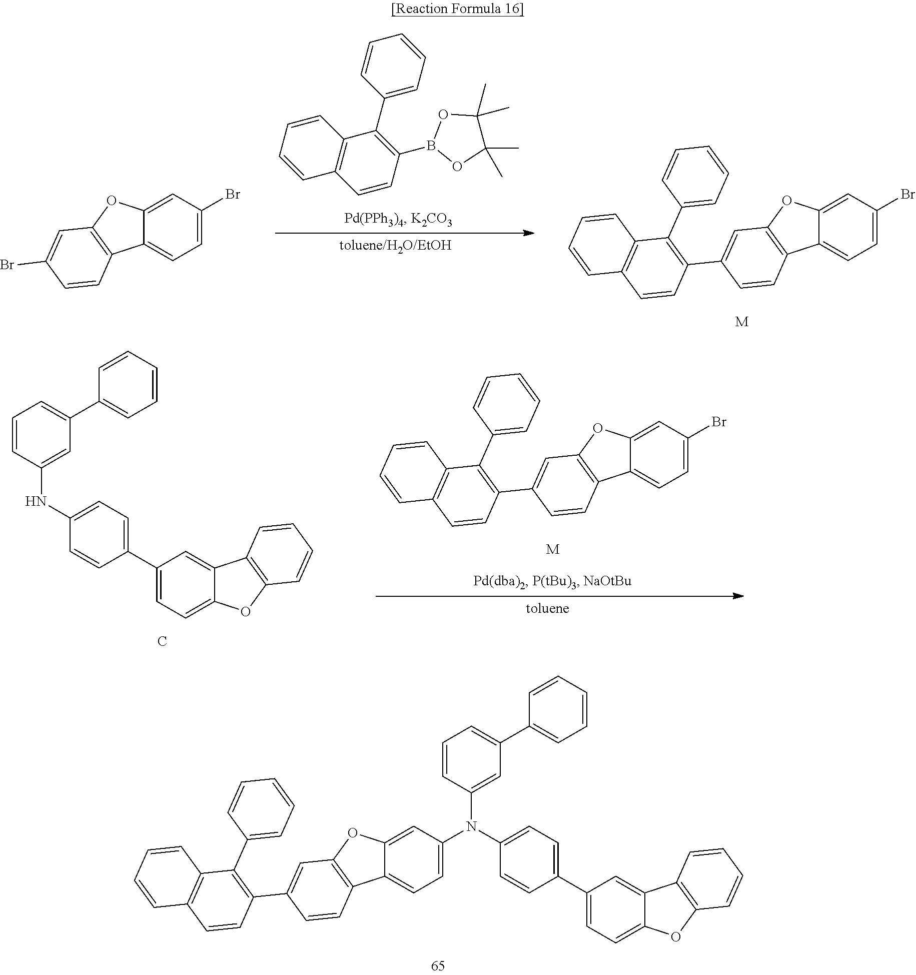

In the same manner as the synthesis of Compound E described above, 8.4 g of Compound M (yield: 61%) was obtained from Intermediate Compound 3,7-dibromodibenzofuran (10.0 g) and 4,4,5,5-tetramethyl-2-(1-phenylnaphthalen-2-yl)-1,3,2-dioxaborolane (10.1 g). A molecular weight of Compound M as determined through FAB-MS was 449.

In the same manner as the synthesis of Compound 2 described above, 5.5 g of Compound 65 (yield: 58%) was obtained from Intermediate Compound C (5.0 g) and M (5.5 g). A molecular weight of Compound 65 as determined through FAB-MS was 779.

### (17) Synthesis of Amine Compound 89

Amine compound 89 according to an aspect may be synthesized by, for example, processes of Reaction Formula 17 below.

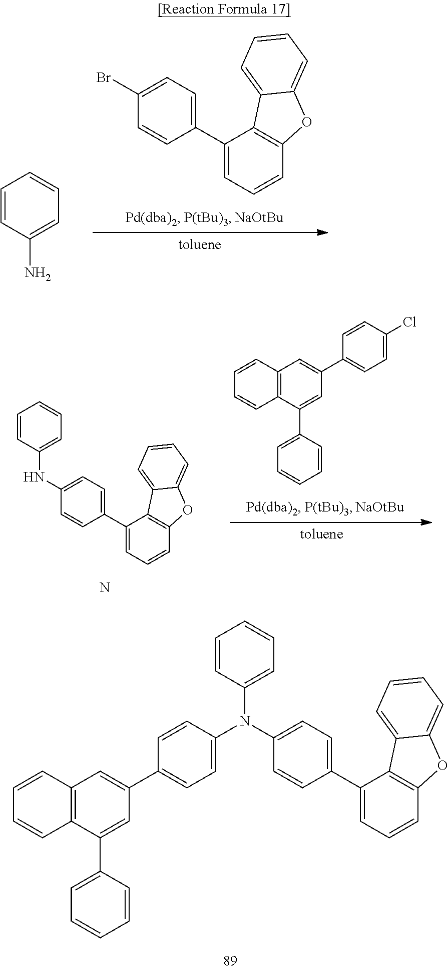

In the same manner as the synthesis of Compound A described above, 5.9 g of Compound N (yield: 55%) was obtained from aniline (3.0 g) and 1-(4-bromophenyl)dibenzofuran (10.4 g). A molecular weight of Compound N as determined through FAB-MS was 335.

In the same manner as the synthesis of Compound 2 described above, 6.7 g of Compound 89 (yield: 73%) was obtained from Intermediate Compound N (5.0 g) and 3-(4-chlorophenyl)-1-phenylnaphthalene (4.7 g). A molecular weight of Compound 89 as determined through FAB-MS was 613.

### (18) Synthesis of Amine Compound 53

Amine compound 53 according to an aspect may be synthesized by, for example, processes of Reaction Formula 18 below.

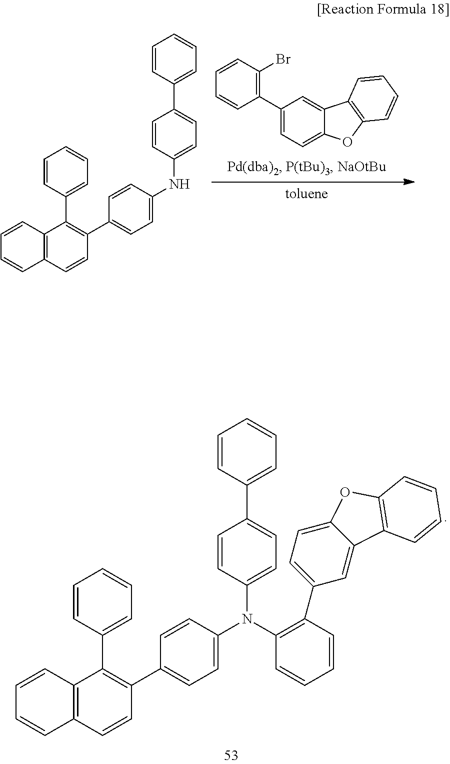

In the same manner as the synthesis of Compound 2 described above, 4.4 g of Compound 53 (yield: 57%) was obtained from Intermediate Compound N-(4-(1-penylnaphthalen-2-yl)phenyl)-[1,1′-biphenyl]-4-amine (5.0 g) and 2-(2-bromophenyl)dibenzofuran (5.4 g). A molecular weight of Compound 53 as determined through FAB-MS was 689.

### (19) Synthesis of Amine Compound 157

Amine compound 157 according to an aspect may be synthesized by, for example, processes of Reaction Formula 19 below.

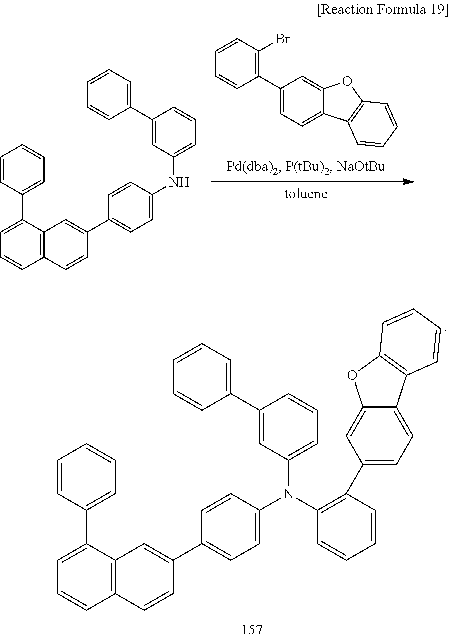 [SMILES: c1ccc(-c2cccc(Nc3ccc(-c4ccc5cccc(-c6ccccc6)c5c4)cc3)c2)cc1 ; Brc1ccccc1-c1ccc2c(c1)oc1ccccc12 ; c1ccc(-c2cccc(N(c3ccc(-c4ccc5cccc(-c6ccccc6)c5c4)cc3)c3ccccc3-c3ccc4c(c3)oc3ccccc34)c2)cc1]

In the same manner as the synthesis of Compound 2 described above, 4.6 g of Compound 157 (yield: 60%) was obtained from Intermediate Compound N-(4-(8-phenylnaphthalen-2-yl)phenyl)-[1,1′-biphenyl]-3-amine (5.0 g) and 3-(2-bromophenyl)dibenzofuran (5.4 g). A molecular weight of Compound 157 as determined through FAB-MS was 689.

### (20) Synthesis of Amine Compound 146

Amine compound 146 according to an aspect may be synthesized by, for example, processes of Reaction Formula 20 below.

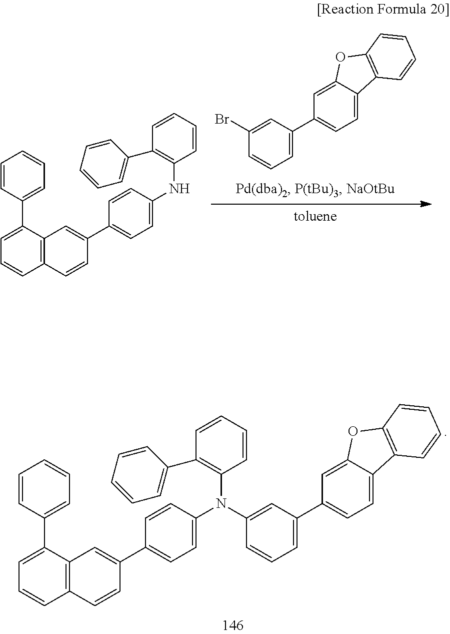

In the same manner as the synthesis of Compound 2 described above, 5.4 g of Compound 146 (yield: 70%) was obtained from Intermediate Compound N-(4-(8-Phenylnaphthalen-2-yl)phenyl)-[1,1′-biphenyl]-2-amine (5.0 g) and 3-(3-bromophenyl)dibenzofuran (3.7 g). A molecular weight of Compound 146 as determined through FAB-MS was 689.

### (21) Synthesis of Amine Compound 212

Amine compound 212 according to an aspect may be synthesized by, for example, processes of Reaction Formula 21 below.

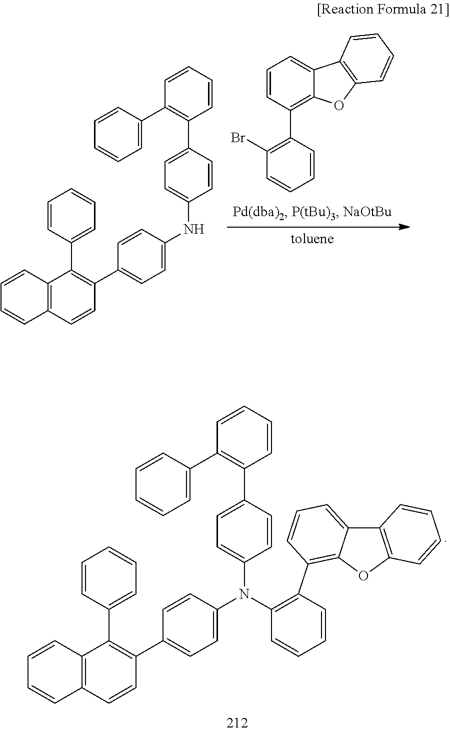

In the same manner as the synthesis of Compound 2 described above, 3.9 g of Compound 212 (yield: 54%) was obtained from Intermediate Compound N-(4-(1-phenylnaphthalen-2-yl)phenyl)-[1,1′:2′,1″-terphenyl]-4-amine (5.0 g) and 4-(2-bromophenyl)dibenzofuran (4.7 g). A molecular weight of Compound 212 as determined through FAB-MS was 765.

### (22) Synthesis of Amine Compound 200

Amine compound 200 according to an aspect may be synthesized by, for example, processes of Reaction Formula 22 below.

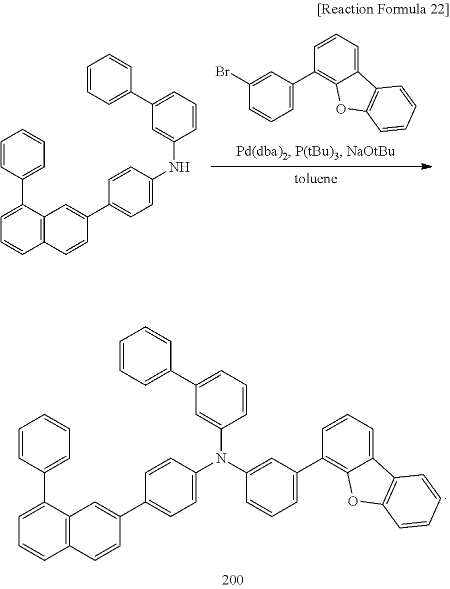 [SMILES: c1ccc(-c2cccc(Nc3ccc(-c4ccc5cccc(-c6ccccc6)c5c4)cc3)c2)cc1 ; Brc1cccc(-c2cccc3c2oc2ccccc23)c1 ; c1ccc(-c2cccc(N(c3ccc(-c4ccc5cccc(-c6ccccc6)c5c4)cc3)c3cccc(-c4cccc5c4oc4ccccc45)c3)c2)cc1]

In the same manner as the synthesis of Compound 2 described above, 5.7 g of Compound 200 (yield: 74%) was obtained from Intermediate Compound N-(4-(8-phenylnaphthalen-2-yl)phenyl)-[1,1′-biphenyl]-3-amine (5.0 g) and 4-(3-bromophenyl)dibenzofuran (3.6 g). A molecular weight of Compound 200 as determined through FAB-MS was 689.

### 2. Preparation and Evaluation of Light Emitting Element

### (1) Preparation of Light Emitting Element

Light emitting elements including amine compounds according to an aspect or Comparative Example compounds in a hole transport layer were prepared through a method below. Light emitting elements of Examples 1 to 22 were prepared using amine Compounds 2, 3, 12, 13, 23, 24, 29, 34, 42, 53, 62, 64, 65, 66, 89, 133, 146, 157, 197, 200, 212, and 400 as a material of the hole transport layer. Light emitting elements of Comparative Examples 1 to 15 were prepared using Comparative Example Compounds X-1 to X-15 as a material of the hole transport layer.

A glass substrate on which an ITO having a thickness of 150 nm was patterned as a first electrode was subjected to ultrasonic cleaning using isopropyl alcohol and pure water each for 5 minutes. The glass substrate was irradiated with UV for 30 minutes, and ozone-treated. Thereafter, a hole injection layer was formed through the deposition of 2-TNATA with a thickness of 60 nm. On the hole injection layer, a hole transport layer was formed through the deposition of Example compounds or Comparative Example compounds with a thickness of 30 nm.

On the hole transport layer, an emission layer was formed through the co-deposition of TBP and ADN with a thickness of 25 nm. TBP and ADN were subjected to the co-deposition at a weight ratio of 3:97. Thereafter, an electron transport region was formed through the sequential deposition of Alq₃ with a thickness of 25 nm and LiF with a thickness of 1 nm. Then, a second electrode was formed through the deposition of Al with a thickness of 100 nm. When preparing a light emitting element, the hole injection layer, the hole transport layer, the emission layer, the electron transport region, and the second electrode were formed using a vacuum deposition device.

The compounds used to prepare the light emitting elements are as follows.

### (Materials Used in Preparation of Light Emitting Elements)

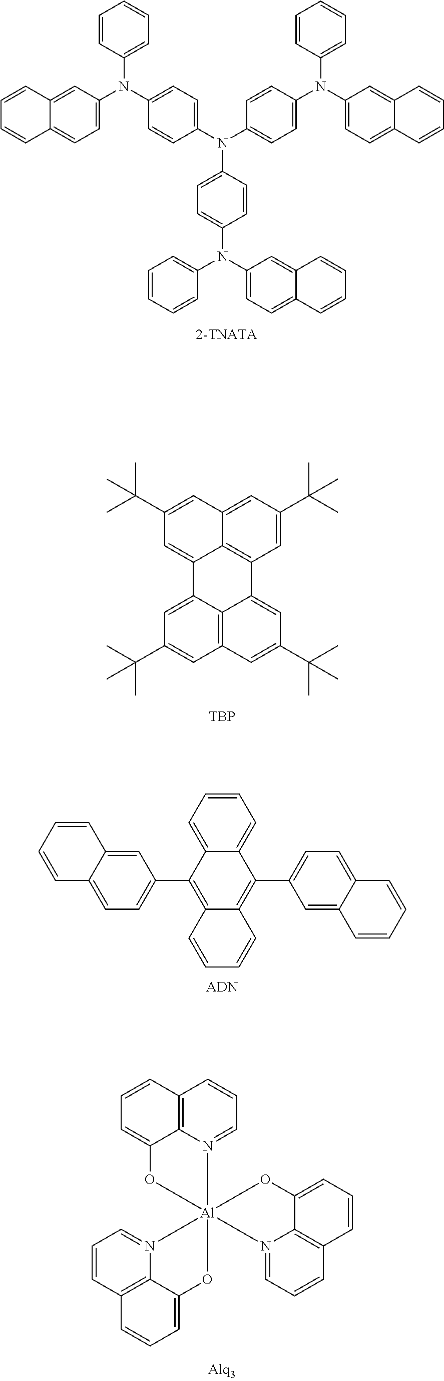

### Example Compound

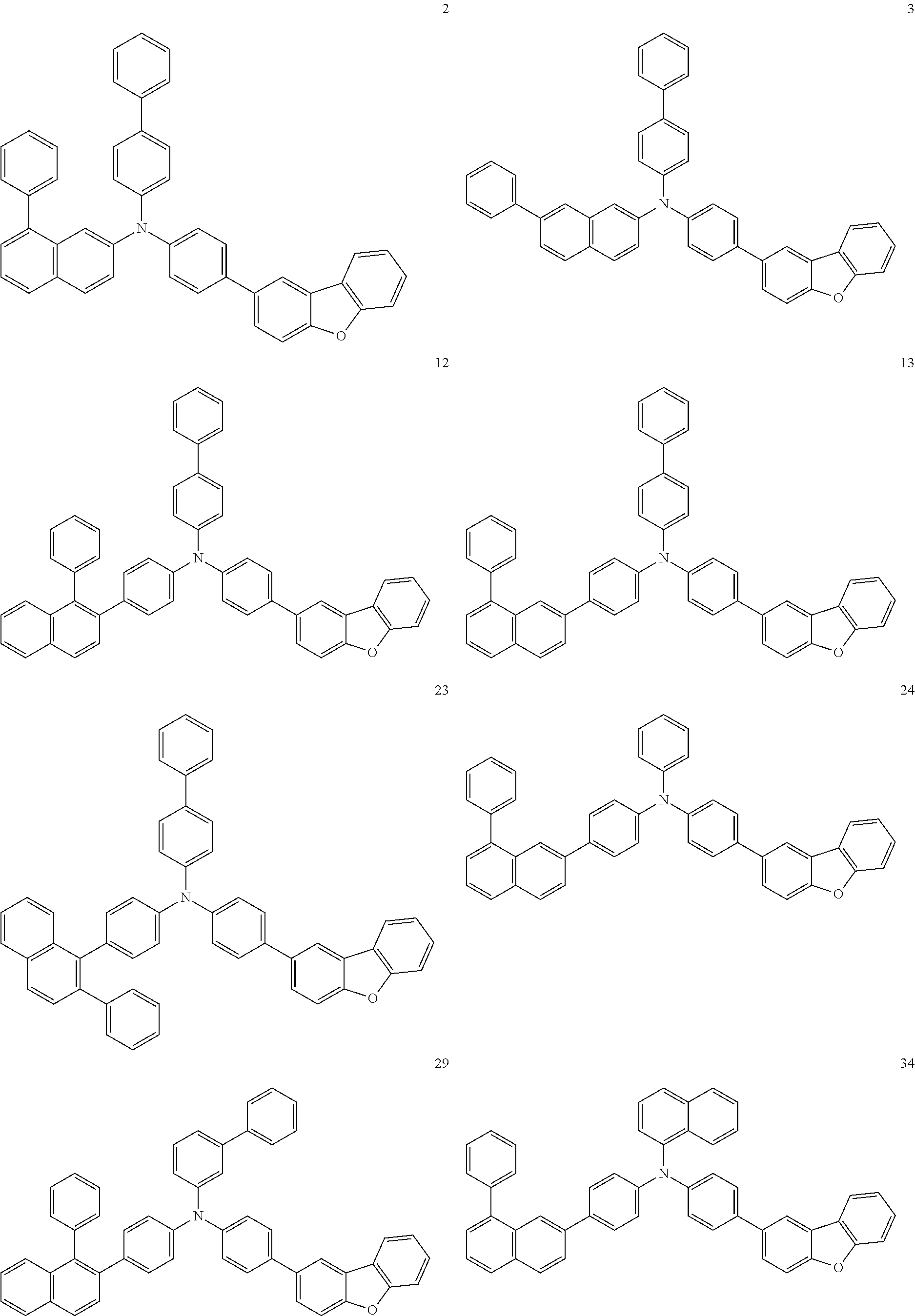

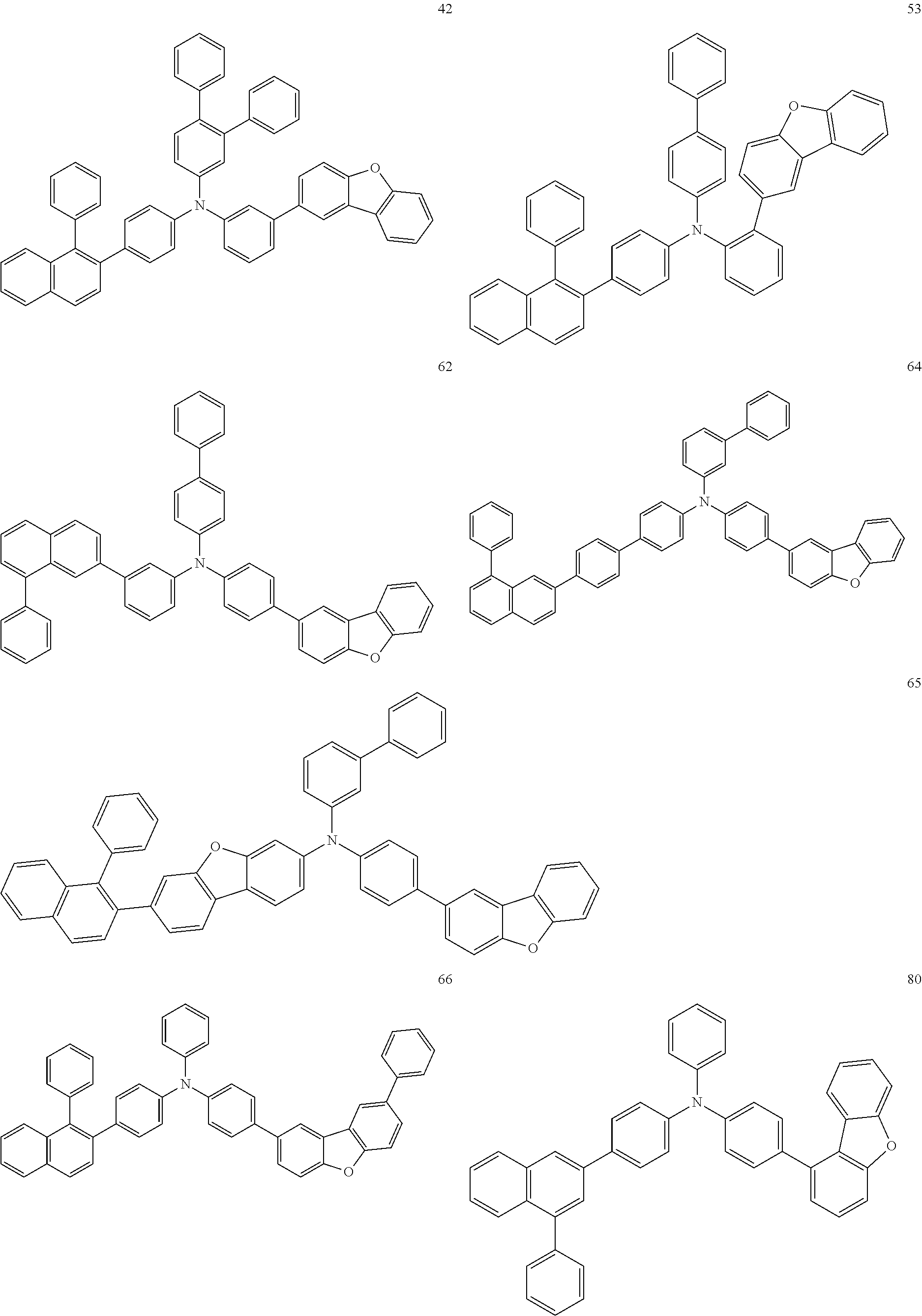

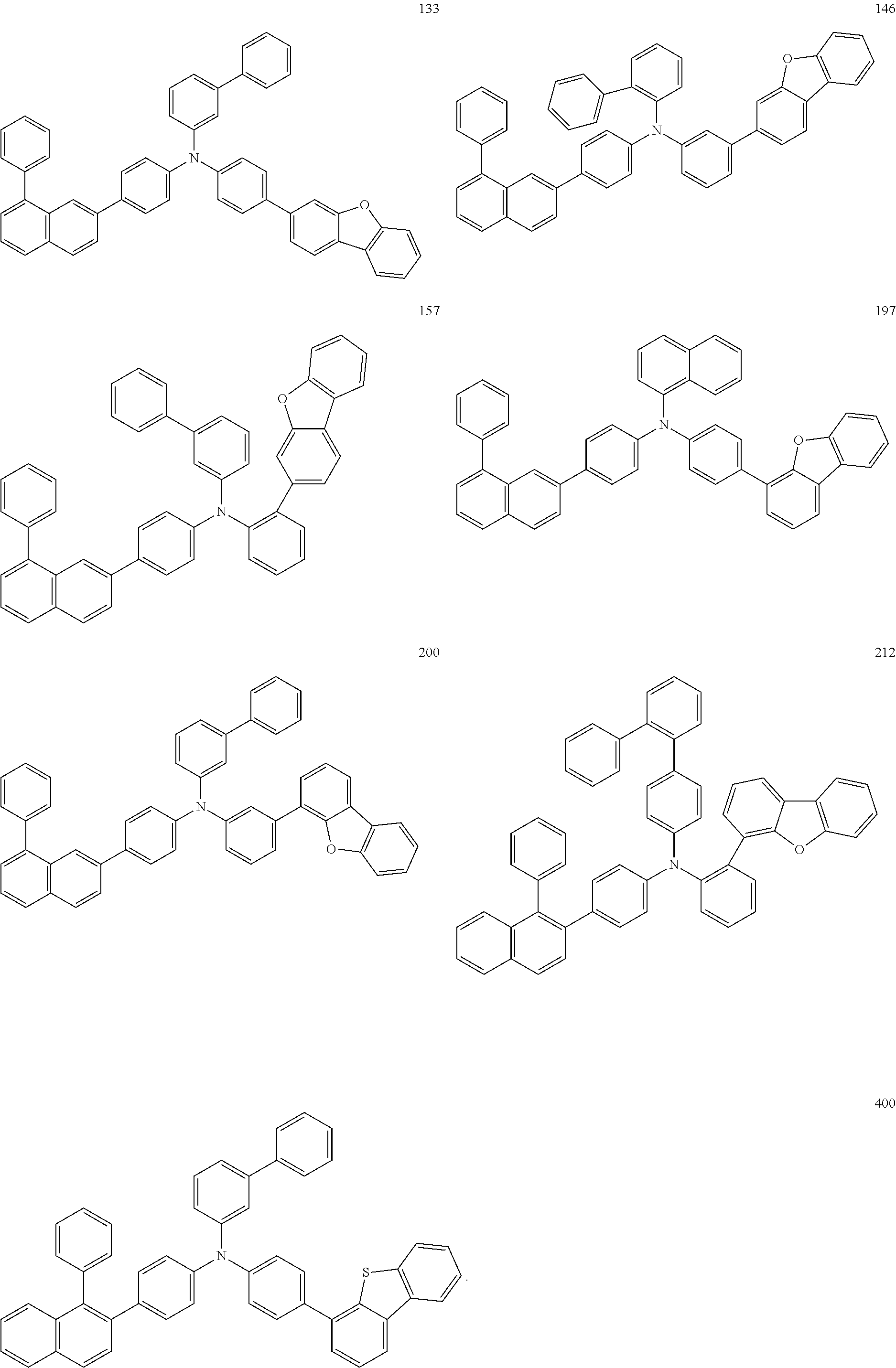

### Comparative Example Compound

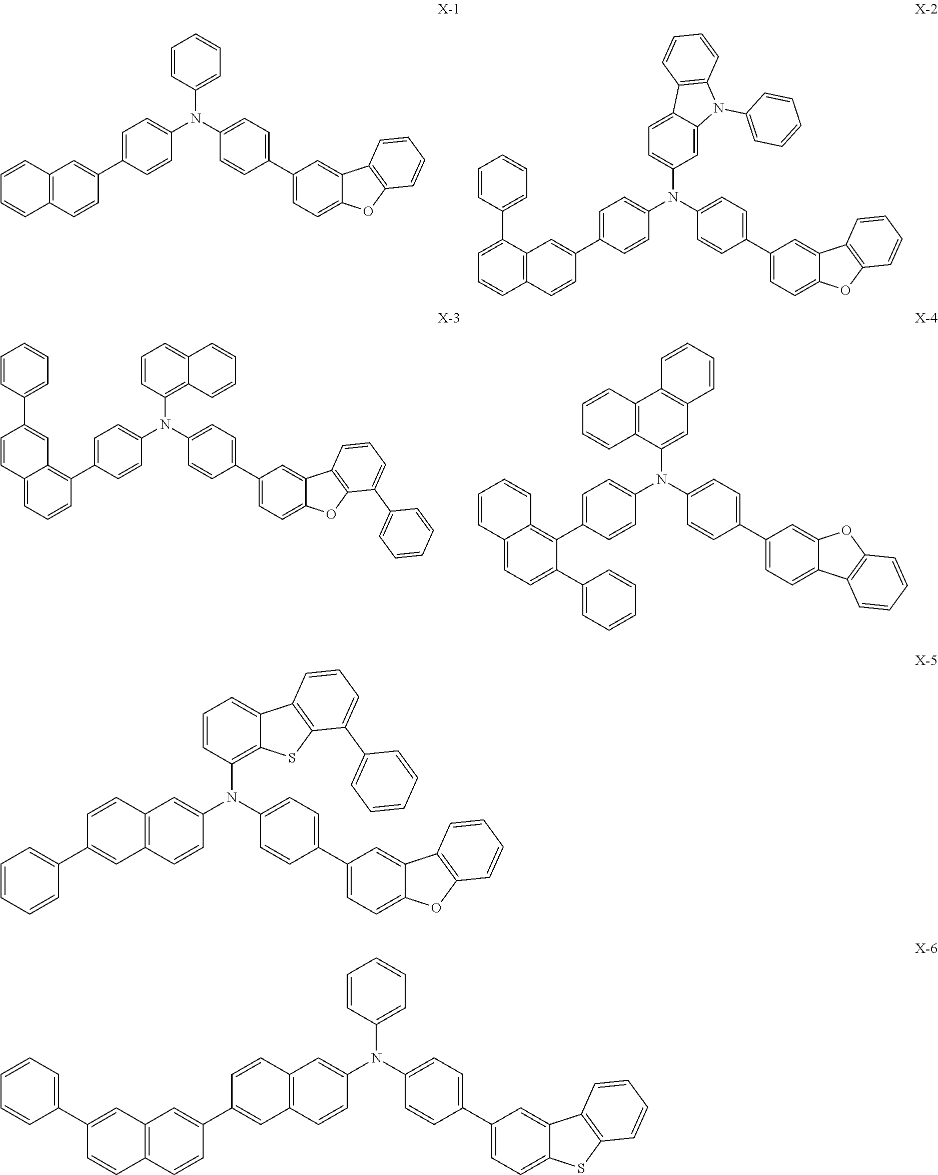

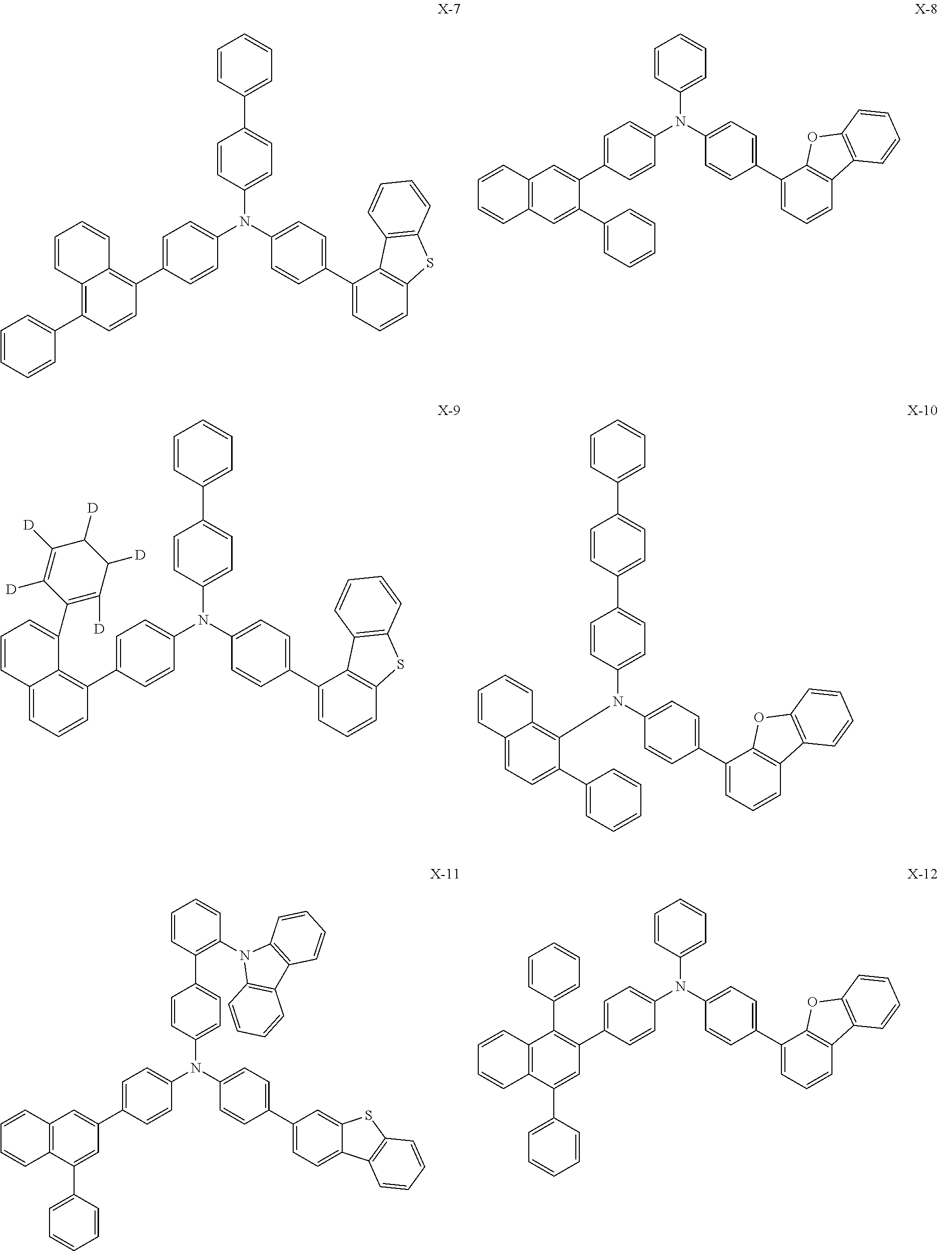

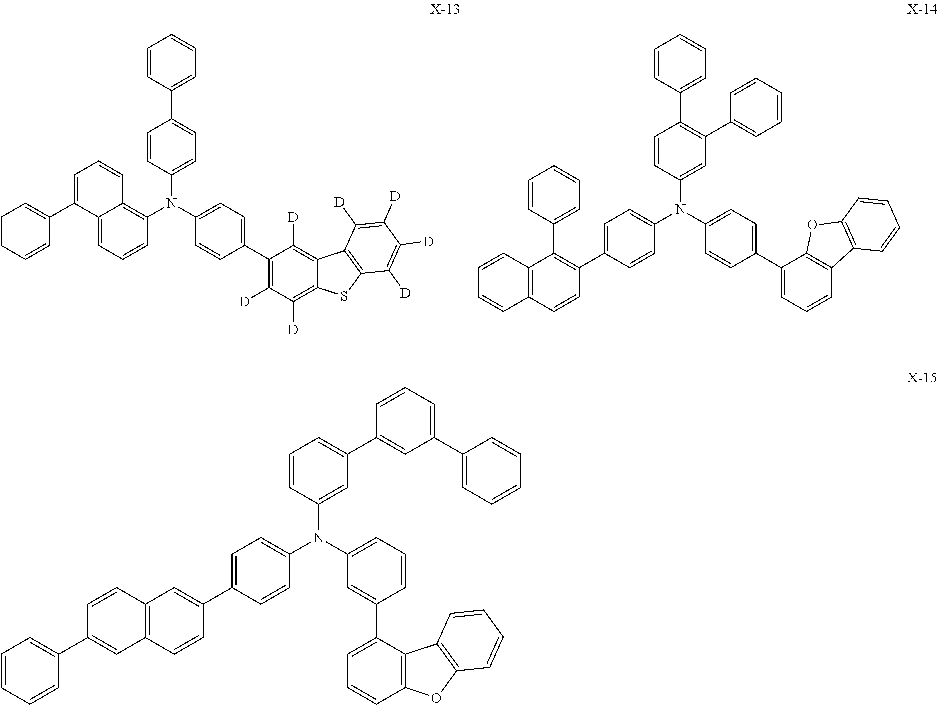 [SMILES: [2H]c1c([2H])c([2H])c2c(sc3c([2H])c([2H])c(-c4ccc(N(c5ccc(-c6ccccc6)cc5)c5cccc6c(C7=CCCC=C7)cccc56)cc4)c([2H])c32)c1[2H] ; c1ccc(-c2ccc(N(c3ccc(-c4ccc5ccccc5c4-c4ccccc4)cc3)c3ccc(-c4cccc5c4oc4ccccc45)cc3)cc2-c2ccccc2)cc1 ; c1ccc(-c2cccc(-c3cccc(N(c4ccc(-c5ccc6cc(-c7ccccc7)ccc6c5)cc4)c4cccc(-c5cccc6oc7ccccc7c56)c4)c3)c2)cc1]

### (2) Evaluation of Light Emitting Element

Table 1 below shows evaluation results of the light emitting elements of Examples and Comparative Examples. Table 1 shows luminous efficiency and element lifespan for the light emitting elements of Examples and Comparative Examples. Luminous efficiency and element lifespan were evaluated using a C9920-11 luminance orientation characteristic measuring device from Hamamatsu Photonics.

Luminous efficiency was shown relatively with respect to luminous efficiency of Comparative Example 1 at a current density of 10 mA/cm² set as 100%. As for relative device lifespan, the time taken for luminance to degrade to 50% from an initial luminance value during continuous operation was shown relatively, with respect to a value of Comparative Example 1 set as 100%.

TABLE 1

| Preparation | | Luminous | Element |
|---|---|---|---|
| example of | Hole transport layer | efficiency | lifespan |
| element | material | (%) | (LT50) (%) |
| Example 1 | Compound 2 | 101% | 120% |
| Example 2 | Compound 12 | 105% | 150% |
| Example 3 | Compound 13 | 103% | 180% |
| Example 4 | Compound 23 | 104% | 150% |
| Example 5 | Compound 24 | 103% | 160% |
| Example 6 | Compound 29 | 106% | 140% |
| Example 7 | Compound 34 | 105% | 140% |
| Example 8 | Compound 42 | 105% | 120% |
| Example 9 | Compound 62 | 104% | 150% |
| Example 10 | Compound 66 | 105% | 130% |
| Example 11 | Compound 133 | 102% | 160% |
| Example 12 | Compound 197 | 105% | 140% |
| Example 13 | Compound 400 | 106% | 130% |
| Example 14 | Compound 3 | 102% | 120% |
| Example 15 | Compound 64 | 106% | 130% |
| Example 16 | Compound 65 | 104% | 120% |
| Example 17 | Compound 89 | 106% | 140% |
| Example 18 | Compound 53 | 107% | 110% |
| Example 19 | Compound 157 | 105% | 120% |
| Example 20 | Compound 146 | 105% | 150% |
| Example 21 | Compound 212 | 106% | 110% |
| Example 22 | Compound 200 | 106% | 140% |
| Comparative | Comparative Example | 100% | 100% |
| Example 1 | compound X-1 | | |
| Comparative | Comparative Example | 95% | 50% |
| Example 2 | compound X-2 | | |
| Comparative | Comparative Example | 98% | 80% |
| Example 3 | compound X-3 | | |
| Comparative | Comparative Example | 101% | 60% |
| Example 4 | compound X-4 | | |
| Comparative | Comparative Example | 98% | 70% |
| Example 5 | compound X-5 | | |
| Comparative | Comparative Example | 99% | 50% |
| Example 6 | compound X-6 | | |
| Comparative | Comparative Example | 99% | 80% |
| Example 7 | compound X-7 | | |
| Comparative | Comparative Example | 99% | 110% |
| Example 8 | compound X-8 | | |
| Comparative | Comparative Example | 98% | 80% |
| Example 9 | compound X-9 | | |
| Comparative | Comparative Example | 98% | 70% |
| Example 10 | compound X-10 | | |
| Comparative | Comparative Example | 99% | 40% |
| Example 11 | compound X-11 | | |
| Comparative | Comparative Example | 100% | 60% |
| Example 12 | compound X-12 | | |
| Comparative | Comparative Example | 100% | 70% |
| Example 13 | compound X-13 | | |
| Comparative | Comparative Example | 101% | 80% |
| Example 14 | Compound X-14 | | |
| Comparative | Comparative Example | 98% | 50% |
| Example 15 | Compound X-15 | | |

Referring to Table 1, Examples 1 to 22 exhibited high efficiency and long lifespan element characteristics compared to Comparative Examples 1 to 15.

Example compounds include first to third substituents that are directly or indirectly bonded to a nitrogen atom, and specifically include a phenylnaphthyl group that is directly bonded to a nitrogen atom at a specific bonding position as a first substituent or bonded to a nitrogen atom through a linker, include a phenyl derivative substituted with a dibenzoheterol group that is directly bonded to a nitrogen atom as a second substituent, and include an aryl derivative that is directly bonded to a nitrogen atom as a third substituent. The third substituent may be a substituted or unsubstituted phenyl group, or a substituted or unsubstituted naphthyl group. Depending on the specific substituent group and substitution position, the compounds may exhibit superior charge transport properties and material stability compared to other Comparative Examples compounds.

That is, it is determined that Example compounds have excellent charge transport properties and charge balance due to the molecular structural characteristics of Example compounds that are distinguished from Comparative Example compounds, and accordingly, the light emitting elements of Examples including the amine compound of Examples in the hole transport layer exhibit high efficiency and long lifespan characteristics.

Specifically, Comparative Compound X-1, unlike Example compounds, includes an unsubstituted naphthyl group instead of a phenylnaphthyl group as the first substituent. It is determined that Comparative Compound X-1 lacks electron tolerance due to the absence of a phenyl group, resulting in degradation in reduced luminous efficiency and element lifespan.

It is determined that Comparative Compound X-2, unlike Example compounds, includes a carbazole group as the third substituent, resulting in degradation in luminous efficiency and element lifespan due to disrupted charge balance.

It is determined that Comparative Example compound X-3, as a compound in which the 2-position of dibenzofuran (the R₁₂ position in Formula 2) is linked to a phenyl group directly bonded to a nitrogen atom, and the 6-position of dibenzofuran (the R₁₅ position in Formula 2) is substituted with a phenyl group, has degradation in luminous efficiency and element lifespan due to disrupted charge balance.

It is determined that Comparative Example compounds X-4, X-6, X-11, and X-12 have high deposition temperatures, resulting in thermal decomposition during deposition, which in turn reduces element lifespan.

Comparative Example compound X-5, unlike Example compounds, includes phenyldibenzothiophene as the third substituent. Comparative Example compound X-5 was found to have suppressed hole injection due to a large volume thereof, resulting in reduced luminous efficiency and element lifespan.

It is determined that Comparative Example compounds X-7, X-8, and X-9 have excessive steric hindrance, resulting in thermal decomposition during a thermal deposition process, which in turn reduces element lifespan.

It is determined that unlike Example compounds, Comparative Example compound X-10 as a compound that includes dibenzofuran-4-ylphenyl (a phenyl derivative in which the compound of Formula 2 is linked to the compound of Formula 1 at the R₁₄ position) as the second substituent and includes phenylnaphthalen-1-yl (R₃₄ of Formula 4 is a phenyl group) as the first substituent, has degradation in luminous efficiency and element lifespan due to low hole mobility.

It is determined that Comparative Example compound X-13, unlike Example compounds, has a different position in which the phenyl group is bonded in the phenylnaphthyl group, which is the first substituent, from Example compounds, resulting in reduced lifespan.

Comparative Example compound X-14, unlike Example compounds, includes dibenzofuran-4-ylphenyl as the second substituent, and includes a phenyl group (branched structure) in which two phenyl groups are substituted as the third substituent. This results in excessive steric hinderance, which in turn reduces lifespan.

Comparative Example compound X-15, unlike Example compounds, has a structure in which 1-dibenzofuran as the second substituent is linked to a phenyl group so as to be positioned meta to the nitrogen atom (the compound of Formula 2 is linked to the compound of Formula 1 at the RR₁₁ position, and the nitrogen atom and the compound of Formula 2 are positioned meta). This results in excessive steric hinderance, which in turn reduces lifespan.

A light emitting element according to an aspect includes an amine compound according to an aspect in a hole transport region, and may thus exhibit high efficiency and long lifespan characteristics.

An amine compound according to an aspect may be used as a material for achieving improved light emitting element characteristics such as high efficiency and long lifespan.

An electronic device according to an aspect may exhibit excellent display quality.

In the above, description has been made with reference to aspects of the present disclosure, but those skilled or of ordinary skill in the art may understand that various modifications and changes may be made to the aspects of the present disclosure insofar as such modifications and changes do not depart from the spirit and technical scope of the present disclosure set forth in the claims to be described later.

Therefore, the technical scope of the present disclosure is not to be limited to the contents stated in the detailed description of the specification, but should be determined by the claims.

What is claimed is:

1. A light emitting element comprising:
a first electrode;
a second electrode disposed on the first electrode;
an emission layer between the first electrode and the second electrode; and
a hole transport region between the first electrode and the emission layer, the hole transport region including an amine compound represented by Formula 1: 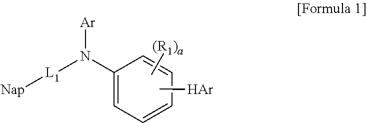
wherein in Formula 1,
L₁ is a direct linkage, a substituted or unsubstituted arylene group having 6 to 30 ring-forming carbon atoms excluding a substituted or unsubstituted naphthyl group, or a substituted or unsubstituted heteroarylene group having 5 to 30 ring-forming carbon atoms,
Ar is a substituted or unsubstituted phenyl group without including an aryl group or a heteroaryl group having 16 or more ring-forming carbon atoms as a substituent, or a substituted or unsubstituted naphthyl group,
a is an integer of 0 to 4,
R₁ is a hydrogen atom, a deuterium atom, a halogen atom, a substituted or unsubstituted alkyl group having 1 to 15 carbon atoms, or a substituted or unsubstituted phenyl group, and
HAr is represented by Formula 2, and Nap is represented by Formula 3 or Formula 4: 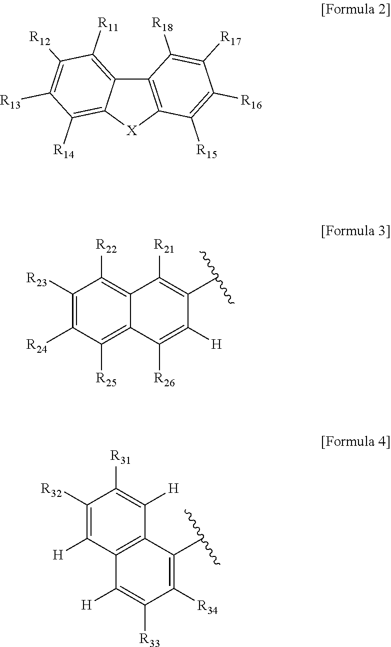
wherein in Formula 2,
X is O or S,
any one of R₁₁ to R₁₄ is a position linked to Formula 1, and the other positions of R₁₁ to R₁₄ excluding the linked position, and R₁₅ to R₁₈ are each independently a hydrogen atom, a deuterium atom, a halogen atom, a substituted or unsubstituted alkyl group having 1 to 15 carbon atoms, or a substituted or unsubstituted phenyl group,
when X is O and when linked to Formula 1 at the R₁₁ position, the R₁₁ position is para to a nitrogen atom of Formula 1,
when linked to Formula 1 at the R₁₂ position, a case in which R₁₅ is a substituted or unsubstituted phenyl group is excluded,
when linked to Formula 1 at the R₁₄ position, Ar of Formula 1 either includes a total of one aryl or heteroaryl group as a substituent or does not include any,
when linked to Formula 1 at the R₁₄ position and when L₁ is a direct linkage, a case in which R₁ is a phenyl group is excluded,
wherein in Formula 3, any one of R₂₁ to R₂₆ is a substituted or unsubstituted phenyl group, and the others are each independently a substituted or unsubstituted alkyl group having 1 to 15 carbon atoms, a halogen atom, a hydrogen atom, or a deuterium atom,
when linked to Formula 1 at the R₁₄ position of Formula 2 and when L₁ is a direct linkage, cases in which R₂₁ or R₂₄ is a substituted or unsubstituted phenyl group are excluded,
wherein in Formula 4, any one of R₃₁ to R₃₄ is a substituted or unsubstituted phenyl group, and the others are each independently a substituted or unsubstituted alkyl group having 1 to 15 carbon atoms, a halogen atom, a hydrogen atom, or a deuterium atom, and
when linked to Formula 1 at the R₁₄ position of Formula 2, a case in which R₃₄ is a substituted or unsubstituted phenyl group is excluded.

2. The light emitting element of claim 1, wherein the hole transport region comprises at least one of a hole injection layer, a hole transport layer, an electron blocking layer, or an auxiliary emission layer, and
at least one of the hole injection layer, the hole transport layer, then electron blocking layer, or then auxiliary emission layer comprises the amine compound.

3. The light emitting element of claim 1, wherein the hole transport region comprises a hole injection layer disposed on the first electrode, and a hole transport layer disposed on the hole injection layer, and
the hole transport layer comprises the amine compound.

4. The light emitting element of claim 1, wherein Formula 1 above is represented by Formula 1-1 or Formula 1-2: 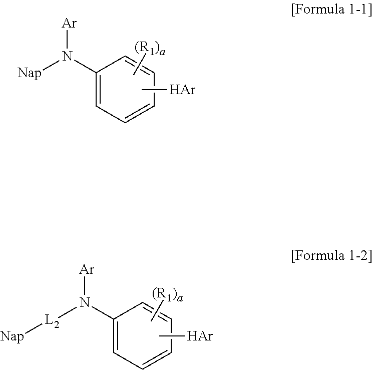
wherein in Formula 1-2, L₂ is a substituted or unsubstituted divalent phenyl, a substituted or unsubstituted divalent biphenyl, or a substituted or unsubstituted divalent dibenzofuran, and
wherein in Formulas 1-1 and 1-2, Nap, Ar, HAr, R₁, and a are the same as defined in Formula 1 above.

5. The light emitting element of claim 1, wherein Ar is a substituted or unsubstituted phenyl group, or a substituted or unsubstituted naphthyl group, and
the substituted or unsubstituted substituent is at least one of a hydrogen atom, a deuterium atom, a halogen atom, a phenyl group, a biphenyl group, or a naphthyl group.

6. The light emitting element of claim 1, wherein at least one hydrogen atom of the amine compound is substituted with a deuterium atom.

7. The light emitting element of claim 1, wherein in Formula 1 above, Nap, L₁, Ar, R₁, and HAr do not comprise a substituted or unsubstituted amine group.

8. The light emitting element of claim 1, wherein the emission layer comprises a compound represented by Formula E-1: 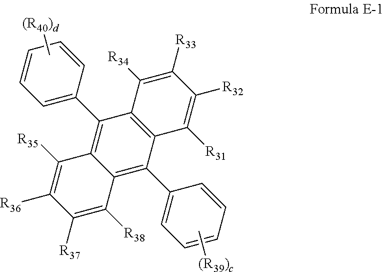
wherein in Formula E-1,
c and d are each independently an integer of 0 to 5, and
R₃₁ to R₄₀ are each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or bonded to an adjacent group to form a ring.

9. The light emitting element of claim 1, wherein the emission layer emits blue light.

10. The light emitting element of claim 1, wherein the amine compound is represented by any one of compounds from Compound Group 1: 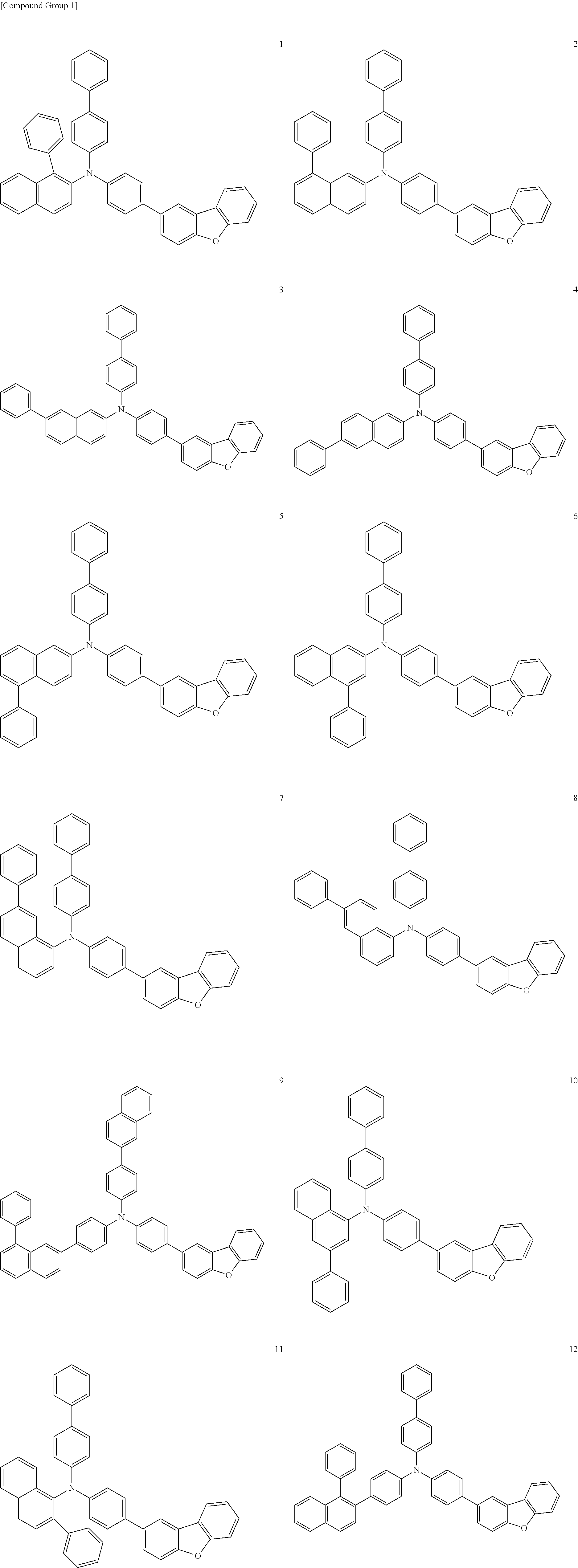 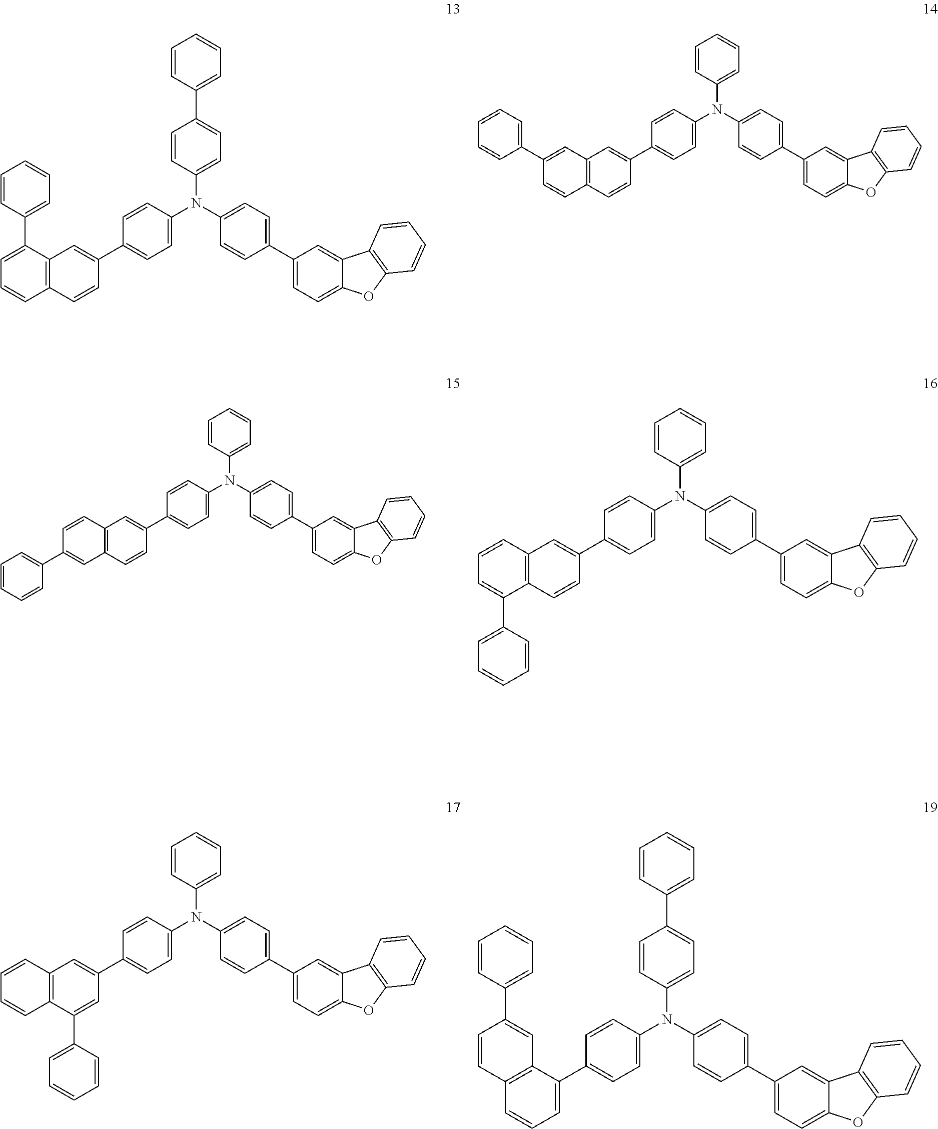 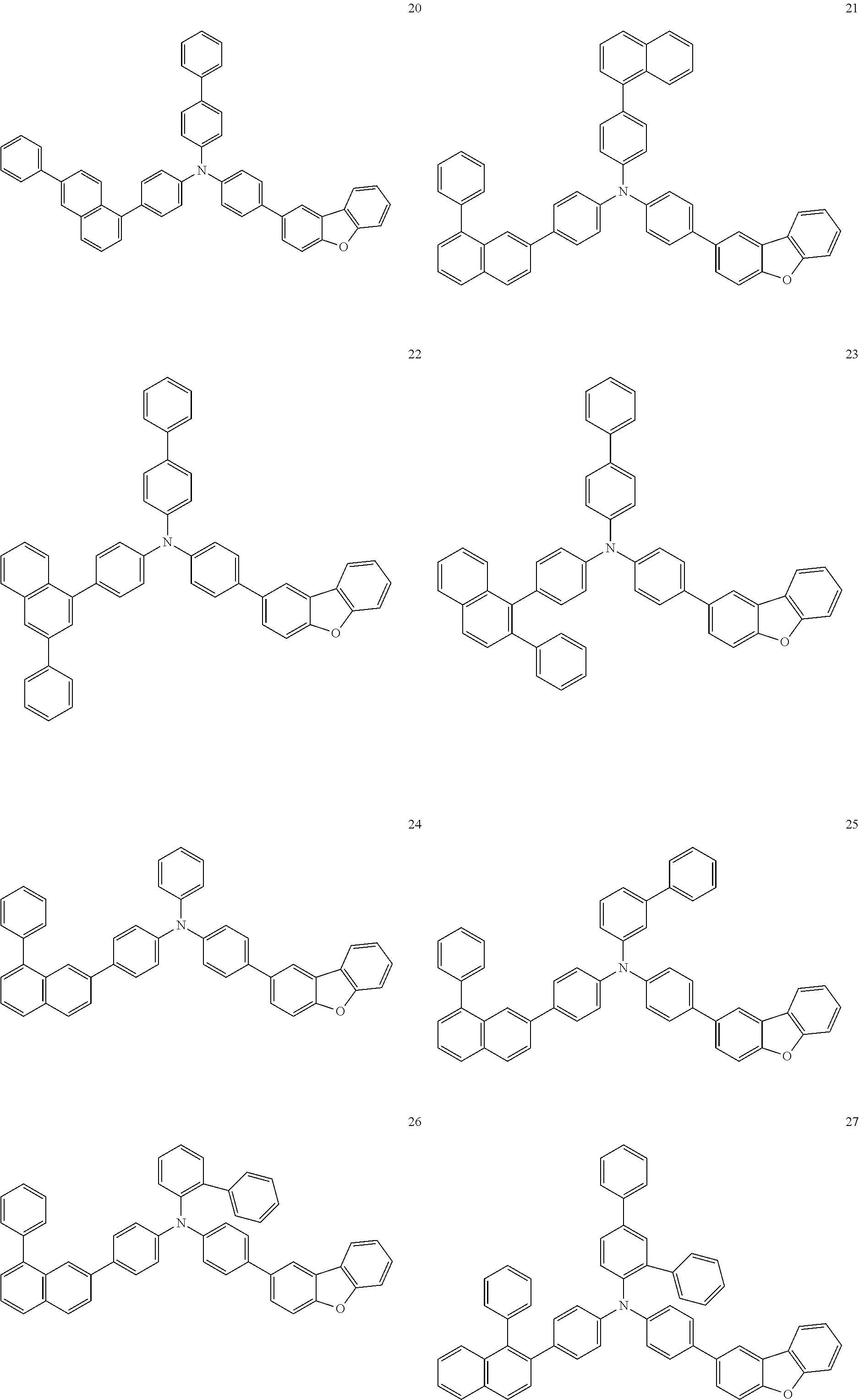 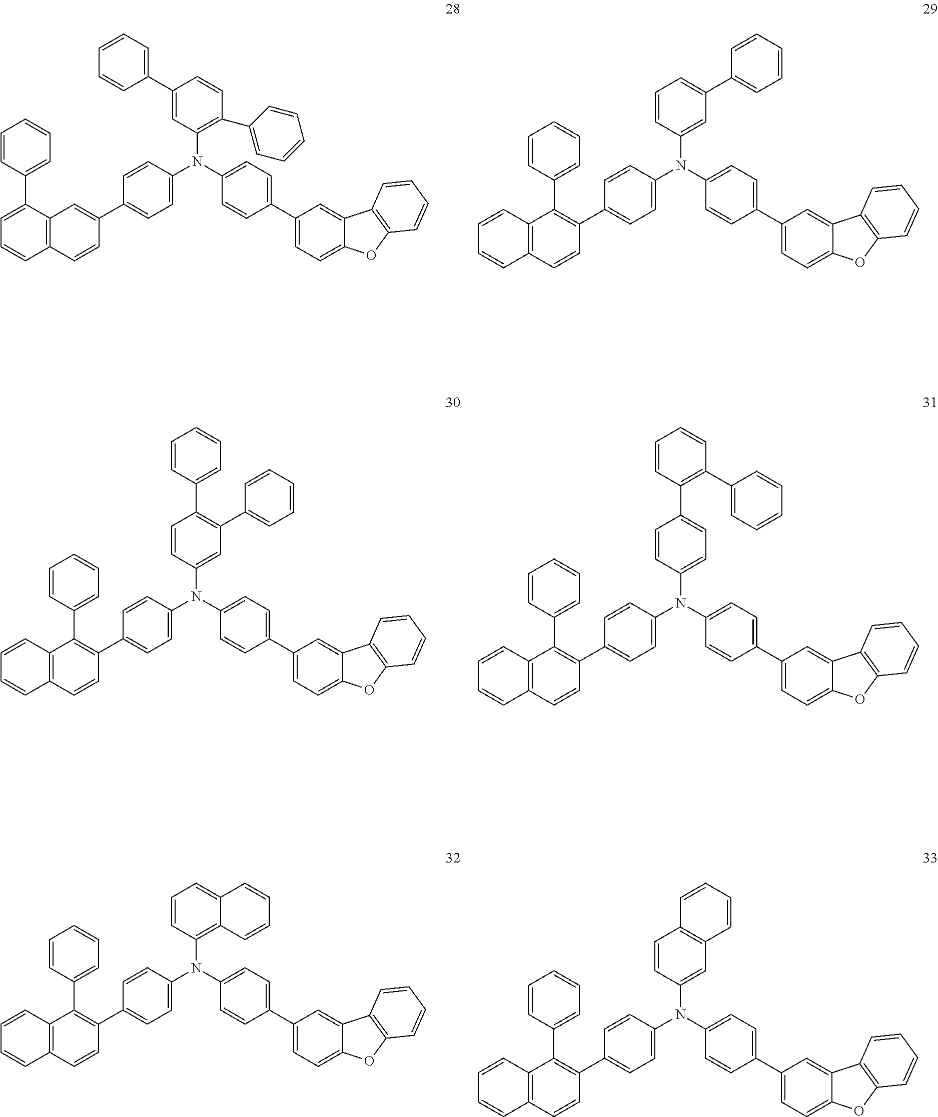 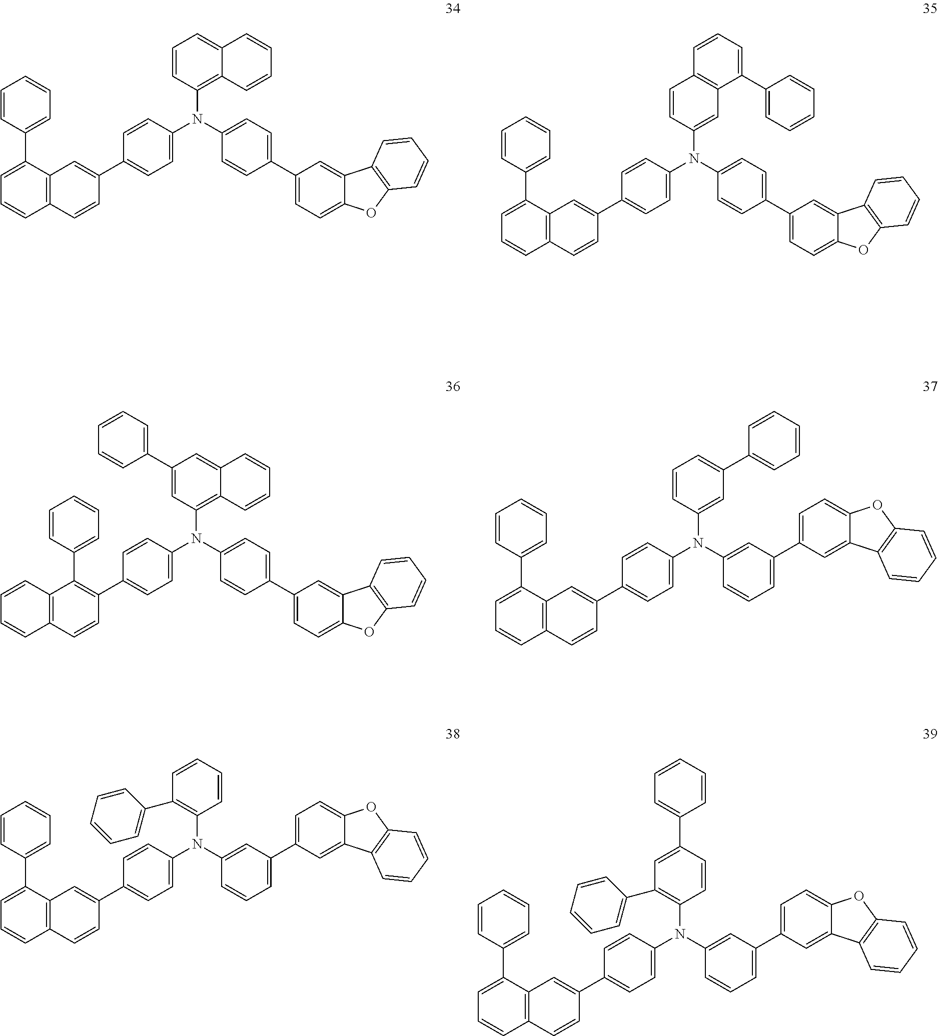 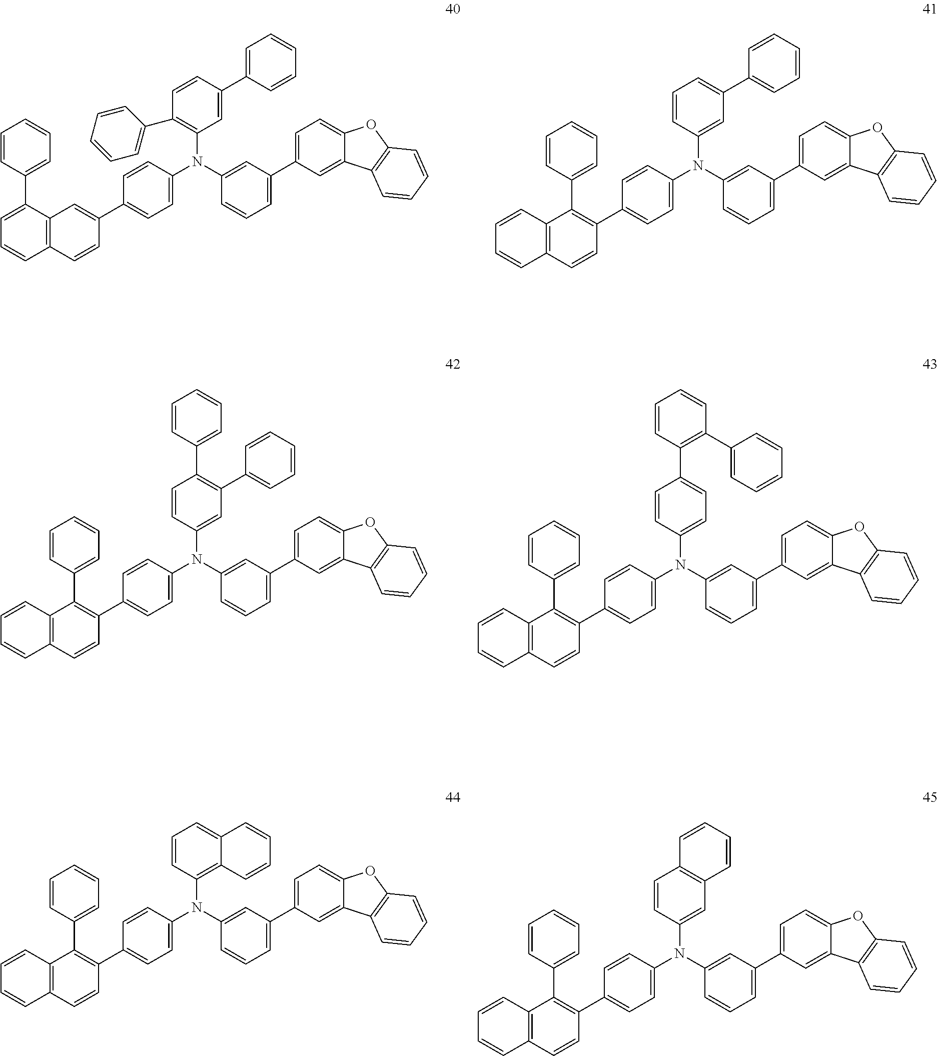 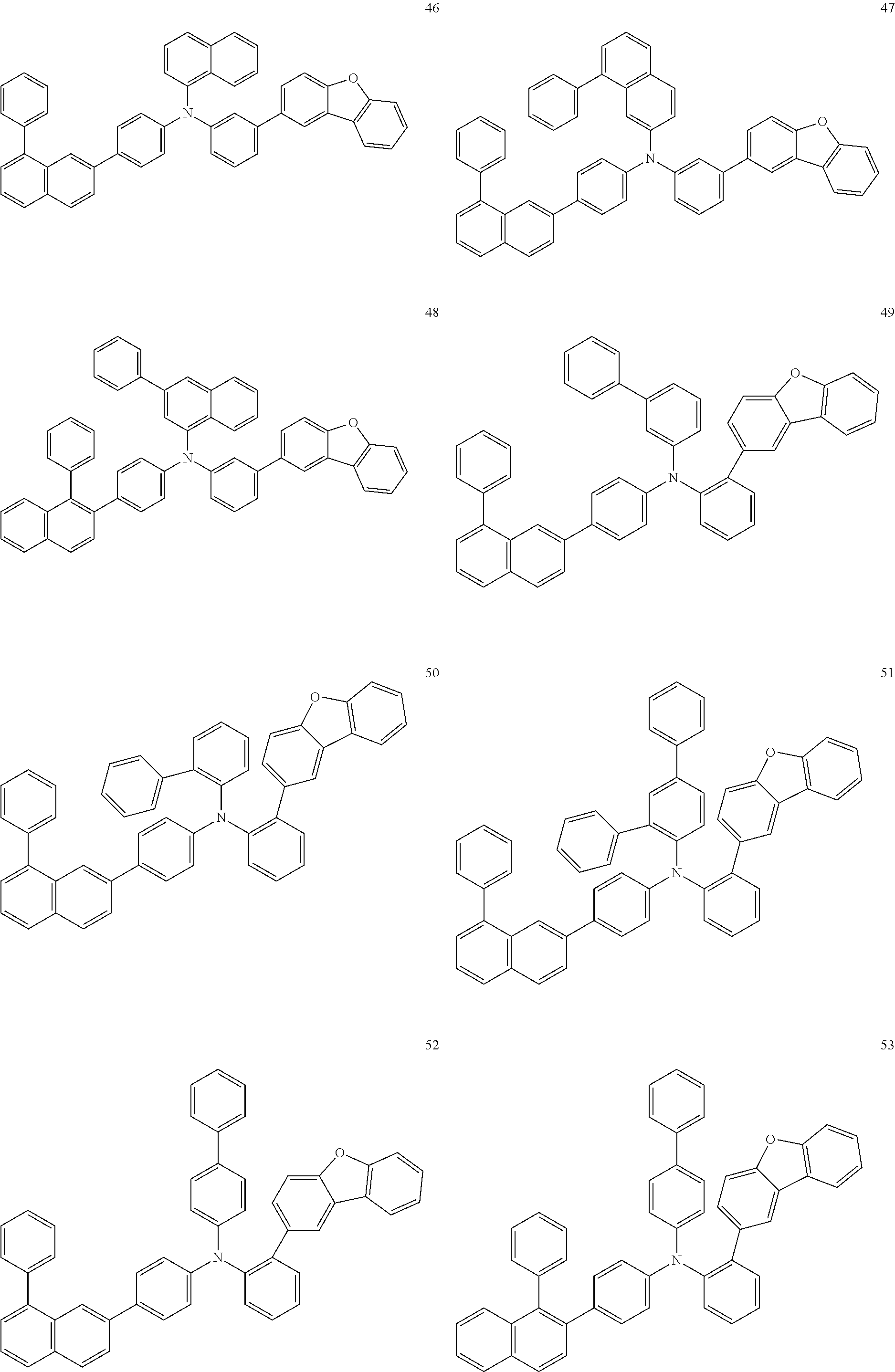 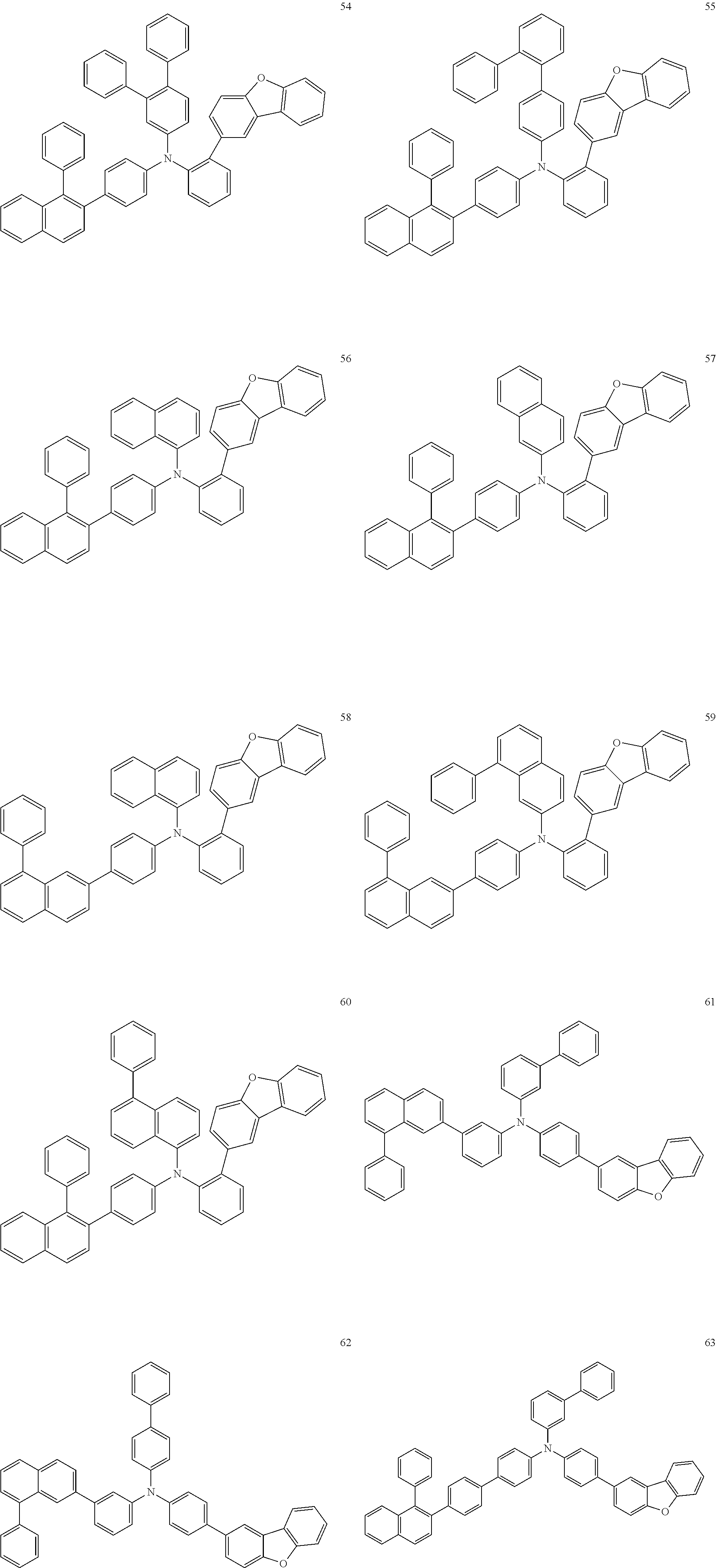 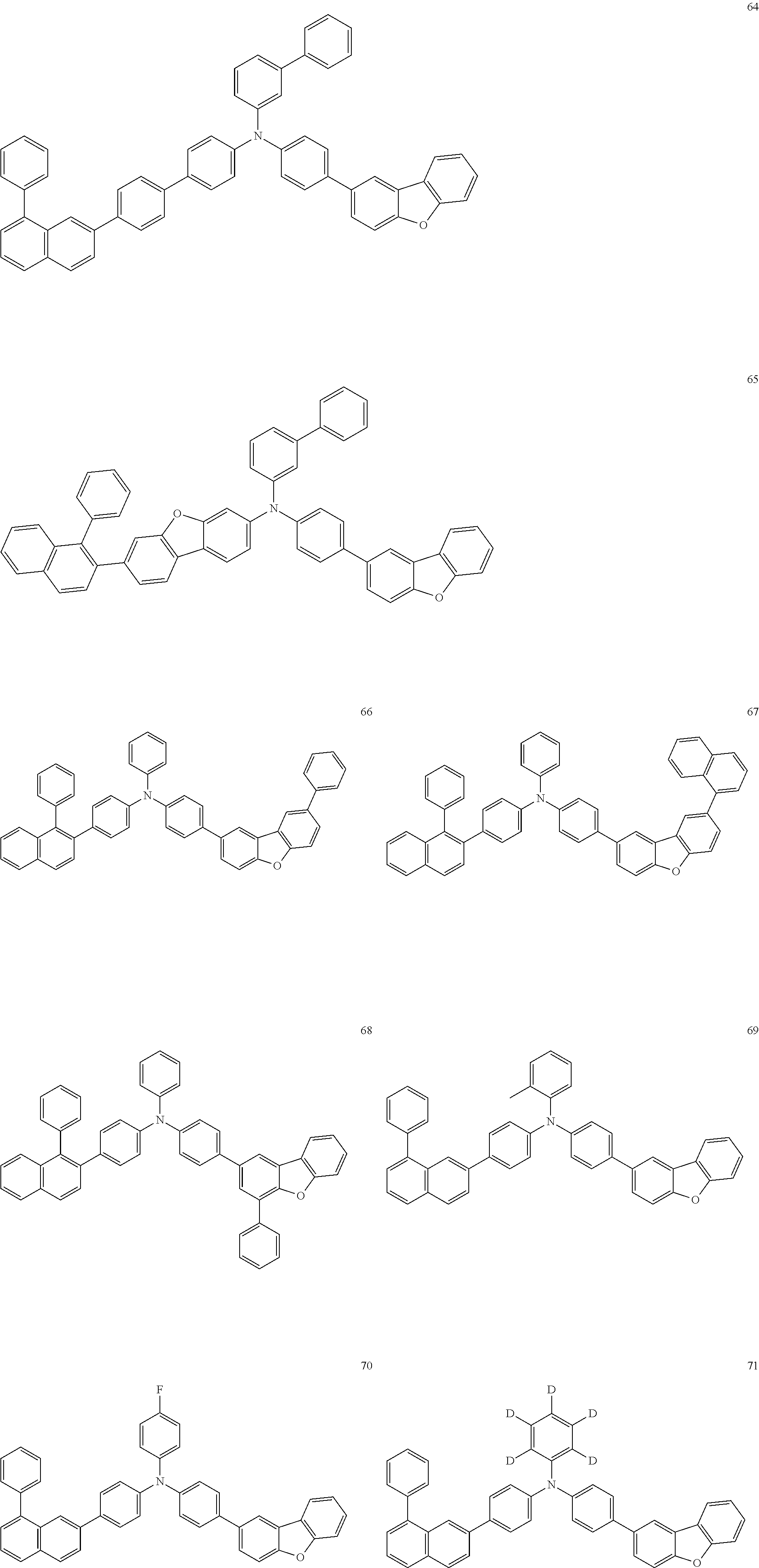 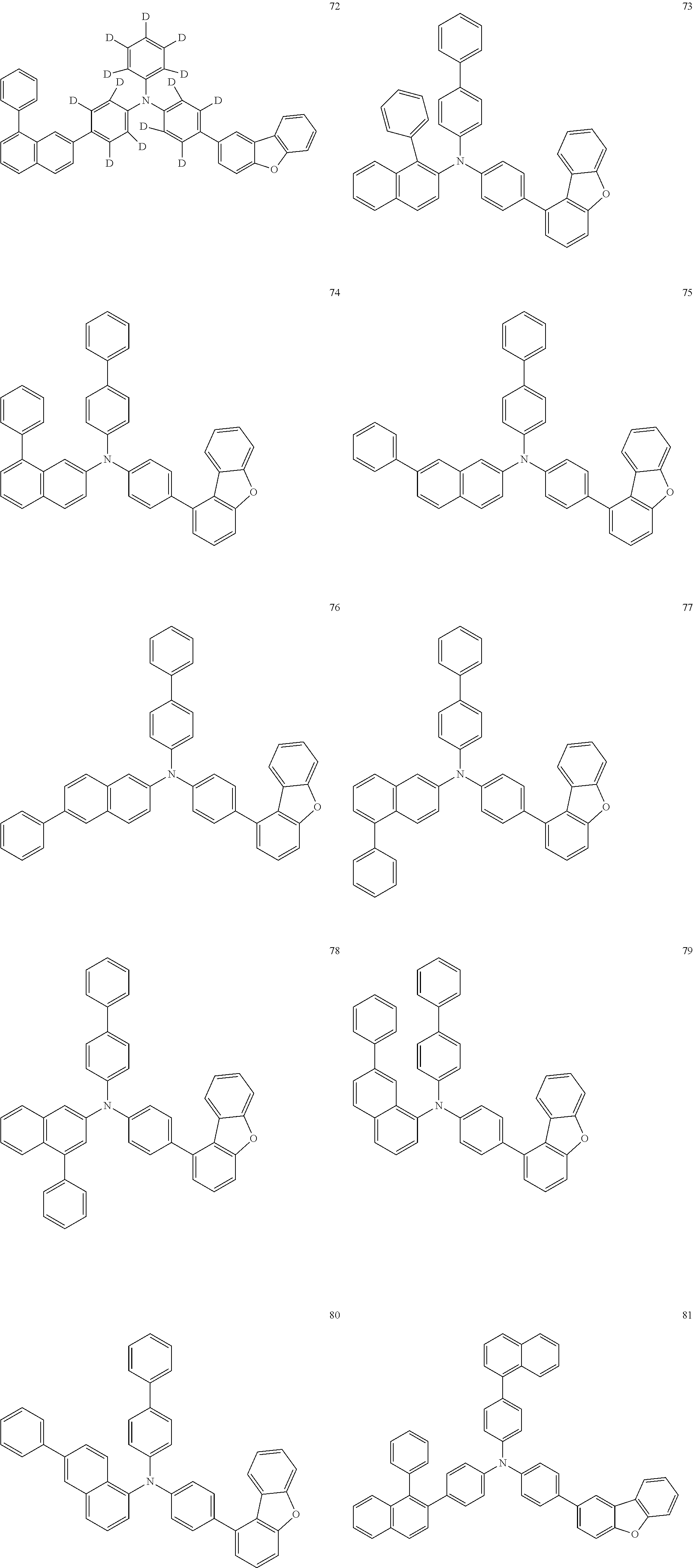 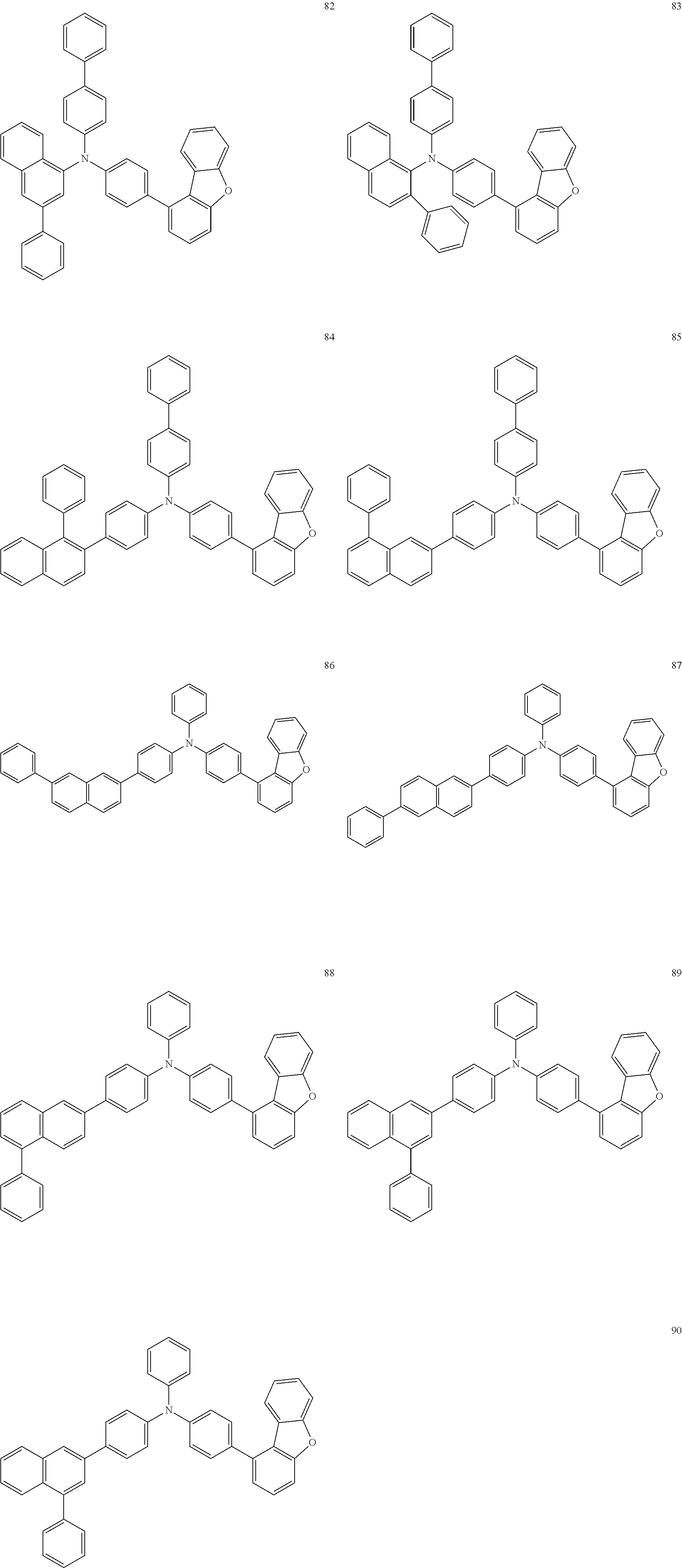 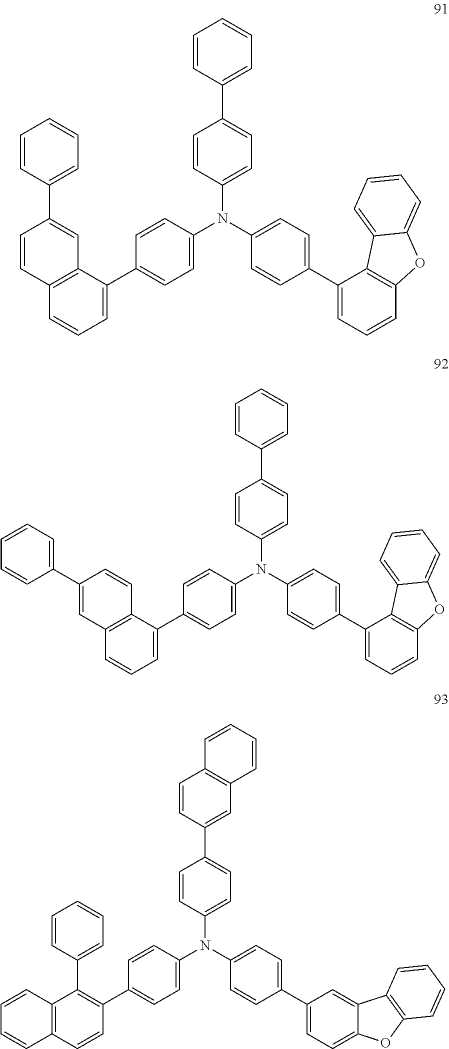 [SMILES: c1ccc(-c2ccc(N(c3ccc(-c4cccc5ccc(-c6ccccc6)cc45)cc3)c3ccc(-c4cccc5oc6ccccc6c45)cc3)cc2)cc1 ; c1ccc(-c2ccc(N(c3ccc(-c4cccc5cc(-c6ccccc6)ccc45)cc3)c3ccc(-c4cccc5oc6ccccc6c45)cc3)cc2)cc1 ; c1ccc(-c2c(-c3ccc(N(c4ccc(-c5ccc6ccccc6c5)cc4)c4ccc(-c5ccc6oc7ccccc7c6c5)cc4)cc3)ccc3ccccc23)cc1] 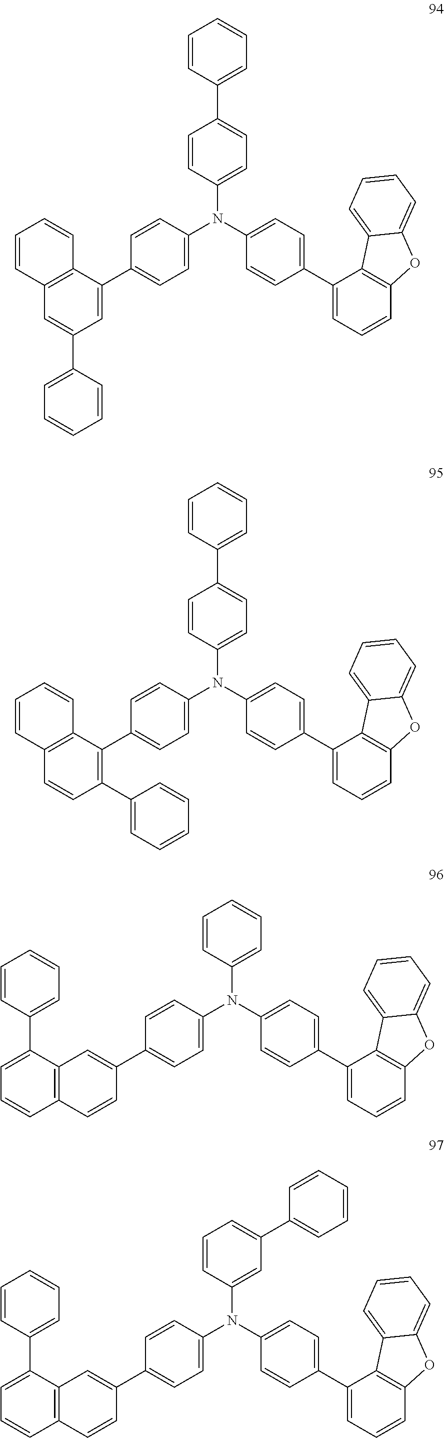 [SMILES: c1ccc(-c2ccc(N(c3ccc(-c4cc(-c5ccccc5)cc5ccccc45)cc3)c3ccc(-c4cccc5oc6ccccc6c45)cc3)cc2)cc1 ; c1ccc(-c2ccc(N(c3ccc(-c4c(-c5ccccc5)ccc5ccccc45)cc3)c3ccc(-c4cccc5oc6ccccc6c45)cc3)cc2)cc1 ; c1ccc(-c2cccc3ccc(-c4ccc(N(c5ccccc5)c5ccc(-c6cccc7oc8ccccc8c67)cc5)cc4)cc23)cc1 ; c1ccc(-c2cccc(N(c3ccc(-c4ccc5cccc(-c6ccccc6)c5c4)cc3)c3ccc(-c4cccc5oc6ccccc6c45)cc3)c2)cc1] 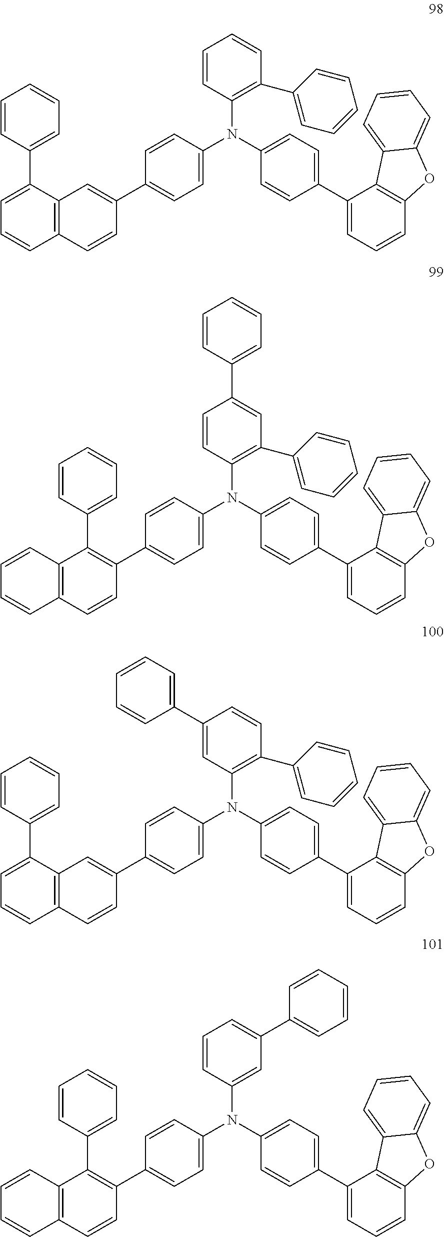 [SMILES: c1ccc(-c2ccccc2N(c2ccc(-c3ccc4cccc(-c5ccccc5)c4c3)cc2)c2ccc(-c3cccc4oc5ccccc5c34)cc2)cc1 ; c1ccc(-c2ccc(N(c3ccc(-c4ccc5ccccc5c4-c4ccccc4)cc3)c3ccc(-c4cccc5oc6ccccc6c45)cc3)c(-c3ccccc3)c2)cc1 ; c1ccc(-c2ccc(-c3ccccc3)c(N(c3ccc(-c4ccc5cccc(-c6ccccc6)c5c4)cc3)c3ccc(-c4cccc5oc6ccccc6c45)cc3)c2)cc1 ; c1ccc(-c2cccc(N(c3ccc(-c4ccc5ccccc5c4-c4ccccc4)cc3)c3ccc(-c4cccc5oc6ccccc6c45)cc3)c2)cc1] 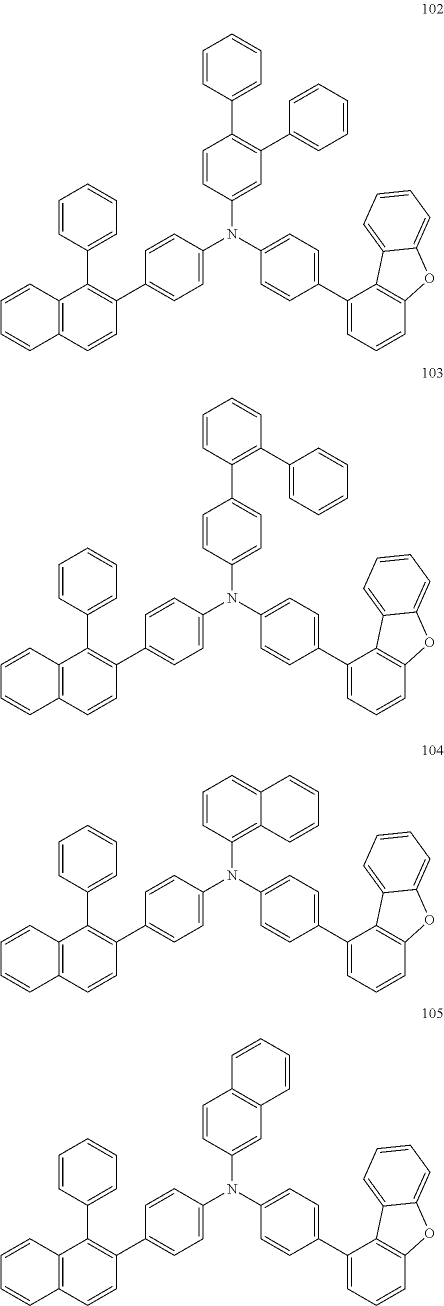 [SMILES: c1ccc(-c2ccc(N(c3ccc(-c4ccc5ccccc5c4-c4ccccc4)cc3)c3ccc(-c4cccc5oc6ccccc6c45)cc3)cc2-c2ccccc2)cc1 ; c1ccc(-c2ccccc2-c2ccc(N(c3ccc(-c4ccc5ccccc5c4-c4ccccc4)cc3)c3ccc(-c4cccc5oc6ccccc6c45)cc3)cc2)cc1 ; c1ccc(-c2c(-c3ccc(N(c4ccc(-c5cccc6oc7ccccc7c56)cc4)c4cccc5ccccc45)cc3)ccc3ccccc23)cc1 ; c1ccc(-c2c(-c3ccc(N(c4ccc(-c5cccc6oc7ccccc7c56)cc4)c4ccc5ccccc5c4)cc3)ccc3ccccc23)cc1] 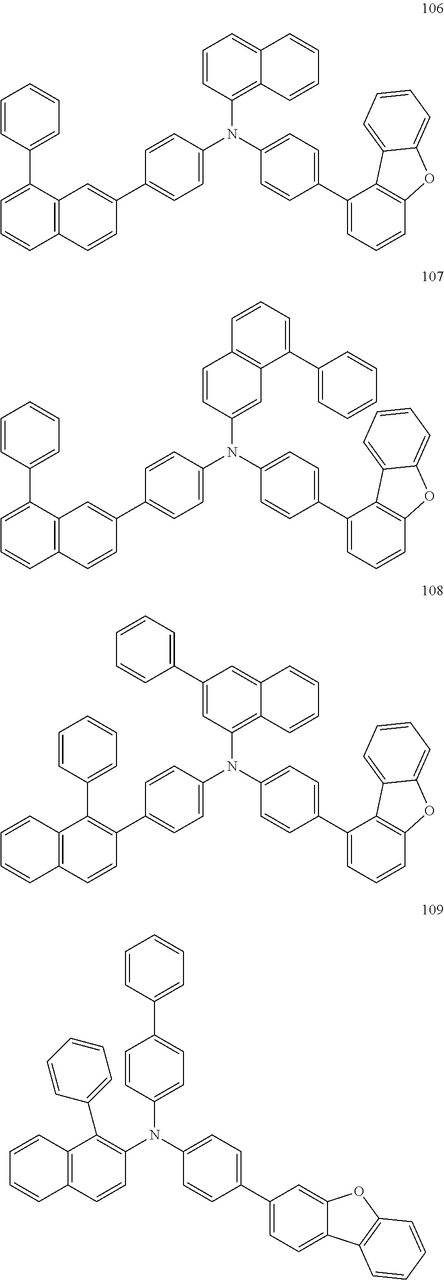 [SMILES: c1ccc(-c2cccc3ccc(-c4ccc(N(c5ccc(-c6cccc7oc8ccccc8c67)cc5)c5cccc6ccccc56)cc4)cc23)cc1 ; c1ccc(-c2cccc3ccc(-c4ccc(N(c5ccc(-c6cccc7oc8ccccc8c67)cc5)c5ccc6cccc(-c7ccccc7)c6c5)cc4)cc23)cc1 ; c1ccc(-c2cc(N(c3ccc(-c4ccc5ccccc5c4-c4ccccc4)cc3)c3ccc(-c4cccc5oc6ccccc6c45)cc3)c3ccccc3c2)cc1 ; c1ccc(-c2ccc(N(c3ccc(-c4ccc5c(c4)oc4ccccc45)cc3)c3ccc4ccccc4c3-c3ccccc3)cc2)cc1] 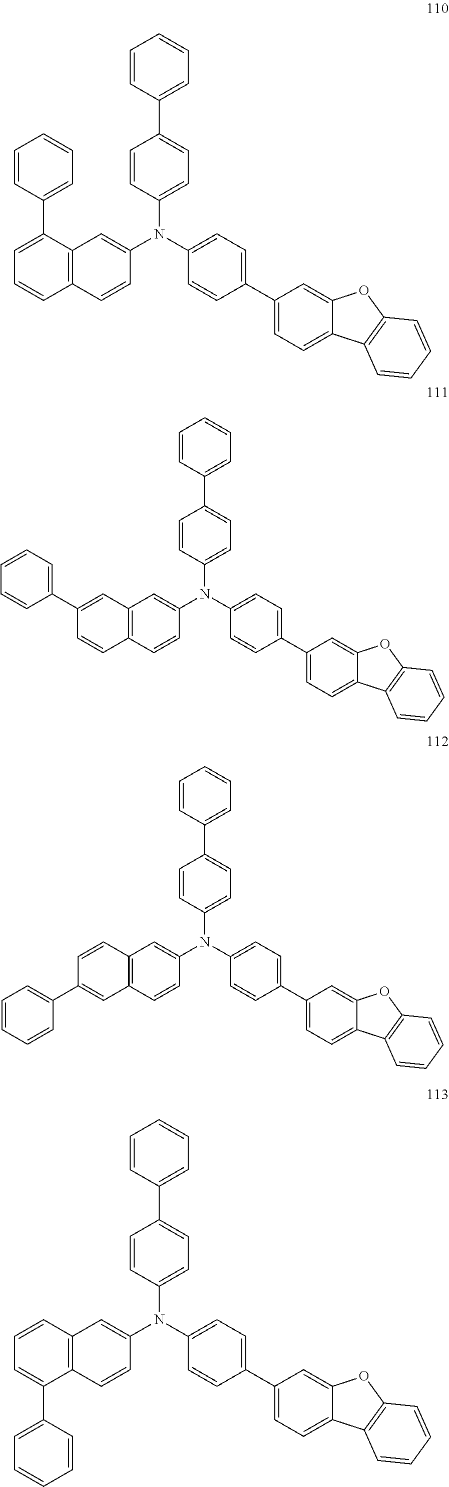 [SMILES: c1ccc(-c2ccc(N(c3ccc(-c4ccc5c(c4)oc4ccccc45)cc3)c3ccc4cccc(-c5ccccc5)c4c3)cc2)cc1 ; c1ccc(-c2ccc(N(c3ccc(-c4ccc5c(c4)oc4ccccc45)cc3)c3ccc4ccc(-c5ccccc5)cc4c3)cc2)cc1 ; c1ccc(-c2ccc(N(c3ccc(-c4ccc5c(c4)oc4ccccc45)cc3)c3ccc4cc(-c5ccccc5)ccc4c3)cc2)cc1 ; c1ccc(-c2ccc(N(c3ccc(-c4ccc5c(c4)oc4ccccc45)cc3)c3ccc4c(-c5ccccc5)cccc4c3)cc2)cc1] 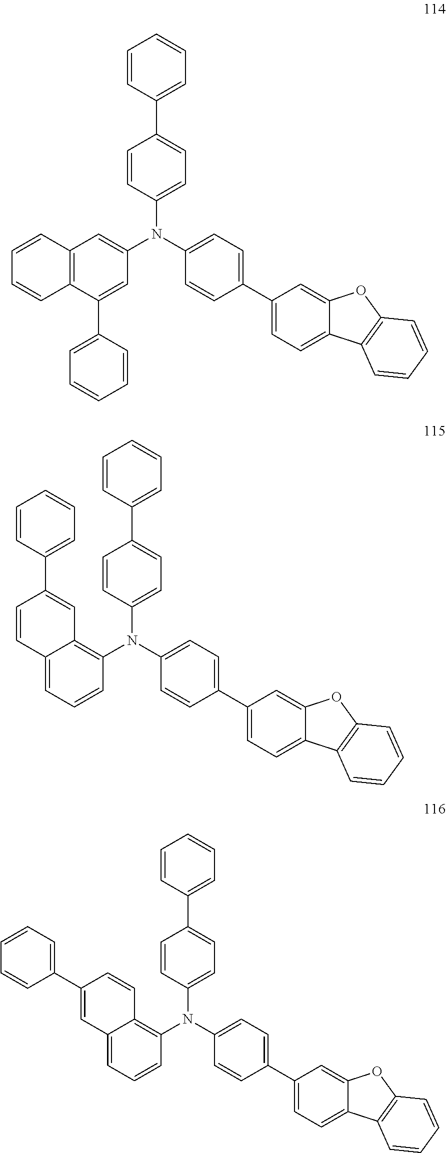 [SMILES: c1ccc(-c2ccc(N(c3ccc(-c4ccc5c(c4)oc4ccccc45)cc3)c3cc(-c4ccccc4)c4ccccc4c3)cc2)cc1 ; c1ccc(-c2ccc(N(c3ccc(-c4ccc5c(c4)oc4ccccc45)cc3)c3cccc4ccc(-c5ccccc5)cc34)cc2)cc1 ; c1ccc(-c2ccc(N(c3ccc(-c4ccc5c(c4)oc4ccccc45)cc3)c3cccc4cc(-c5ccccc5)ccc34)cc2)cc1] 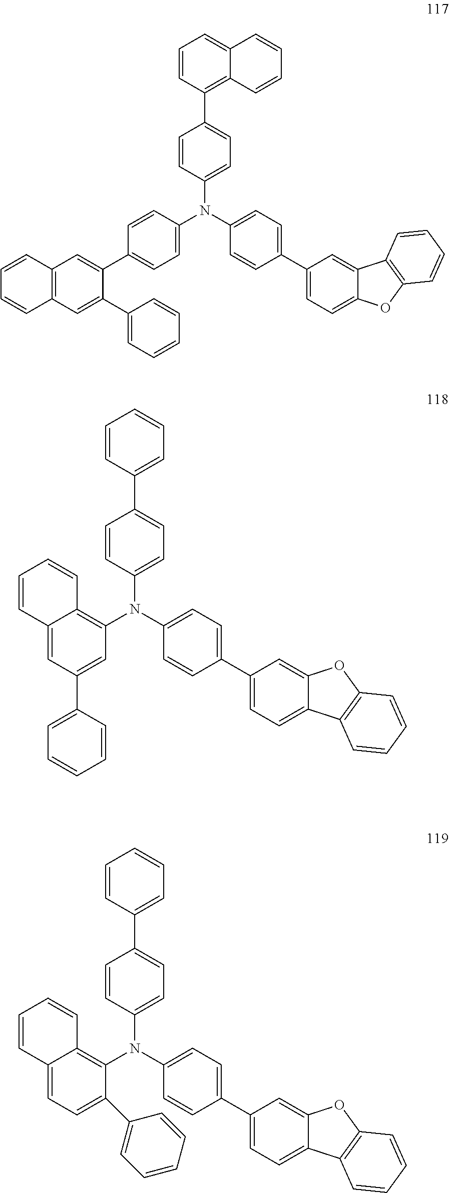 [SMILES: c1ccc(-c2cc3ccccc3cc2-c2ccc(N(c3ccc(-c4ccc5oc6ccccc6c5c4)cc3)c3ccc(-c4cccc5ccccc45)cc3)cc2)cc1 ; c1ccc(-c2ccc(N(c3ccc(-c4ccc5c(c4)oc4ccccc45)cc3)c3cc(-c4ccccc4)cc4ccccc34)cc2)cc1 ; c1ccc(-c2ccc(N(c3ccc(-c4ccc5c(c4)oc4ccccc45)cc3)c3c(-c4ccccc4)ccc4ccccc34)cc2)cc1] 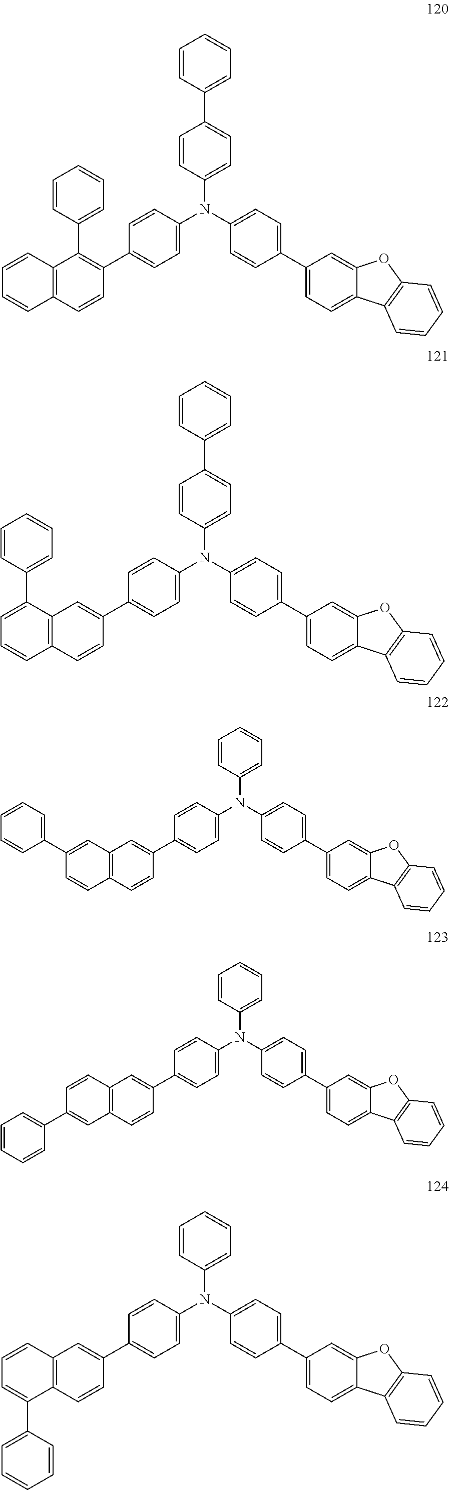 [SMILES: c1ccc(-c2ccc(N(c3ccc(-c4ccc5c(c4)oc4ccccc45)cc3)c3ccc(-c4ccc5ccccc5c4-c4ccccc4)cc3)cc2)cc1 ; c1ccc(-c2ccc(N(c3ccc(-c4ccc5c(c4)oc4ccccc45)cc3)c3ccc(-c4ccc5cccc(-c6ccccc6)c5c4)cc3)cc2)cc1 ; c1ccc(-c2ccc3ccc(-c4ccc(N(c5ccccc5)c5ccc(-c6ccc7c(c6)oc6ccccc67)cc5)cc4)cc3c2)cc1 ; c1ccc(-c2ccc3cc(-c4ccc(N(c5ccccc5)c5ccc(-c6ccc7c(c6)oc6ccccc67)cc5)cc4)ccc3c2)cc1 ; c1ccc(-c2cccc3cc(-c4ccc(N(c5ccccc5)c5ccc(-c6ccc7c(c6)oc6ccccc67)cc5)cc4)ccc23)cc1] 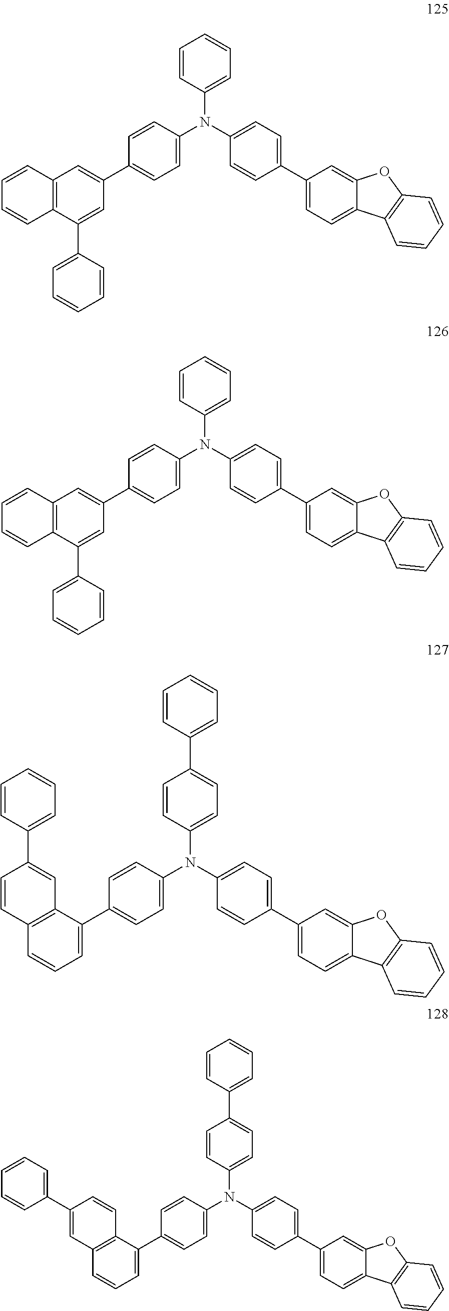 [SMILES: c1ccc(-c2cc(-c3ccc(N(c4ccccc4)c4ccc(-c5ccc6c(c5)oc5ccccc56)cc4)cc3)cc3ccccc23)cc1 ; c1ccc(-c2cc(-c3ccc(N(c4ccccc4)c4ccc(-c5ccc6c(c5)oc5ccccc56)cc4)cc3)cc3ccccc23)cc1 ; c1ccc(-c2ccc(N(c3ccc(-c4ccc5c(c4)oc4ccccc45)cc3)c3ccc(-c4cccc5ccc(-c6ccccc6)cc45)cc3)cc2)cc1 ; c1ccc(-c2ccc(N(c3ccc(-c4ccc5c(c4)oc4ccccc45)cc3)c3ccc(-c4cccc5cc(-c6ccccc6)ccc45)cc3)cc2)cc1] 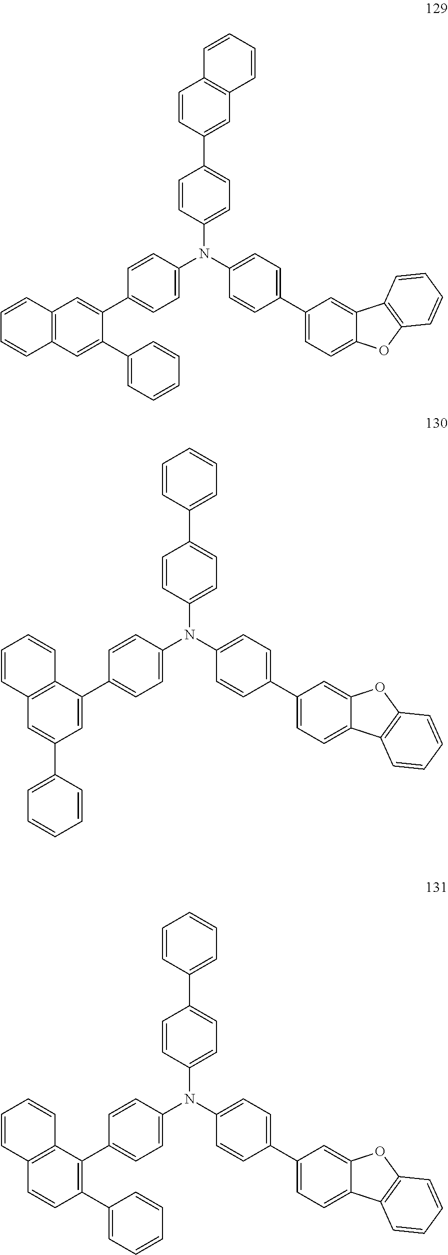 [SMILES: c1ccc(-c2cc3ccccc3cc2-c2ccc(N(c3ccc(-c4ccc5ccccc5c4)cc3)c3ccc(-c4ccc5oc6ccccc6c5c4)cc3)cc2)cc1 ; c1ccc(-c2ccc(N(c3ccc(-c4ccc5c(c4)oc4ccccc45)cc3)c3ccc(-c4cc(-c5ccccc5)cc5ccccc45)cc3)cc2)cc1 ; c1ccc(-c2ccc(N(c3ccc(-c4ccc5c(c4)oc4ccccc45)cc3)c3ccc(-c4c(-c5ccccc5)ccc5ccccc45)cc3)cc2)cc1] 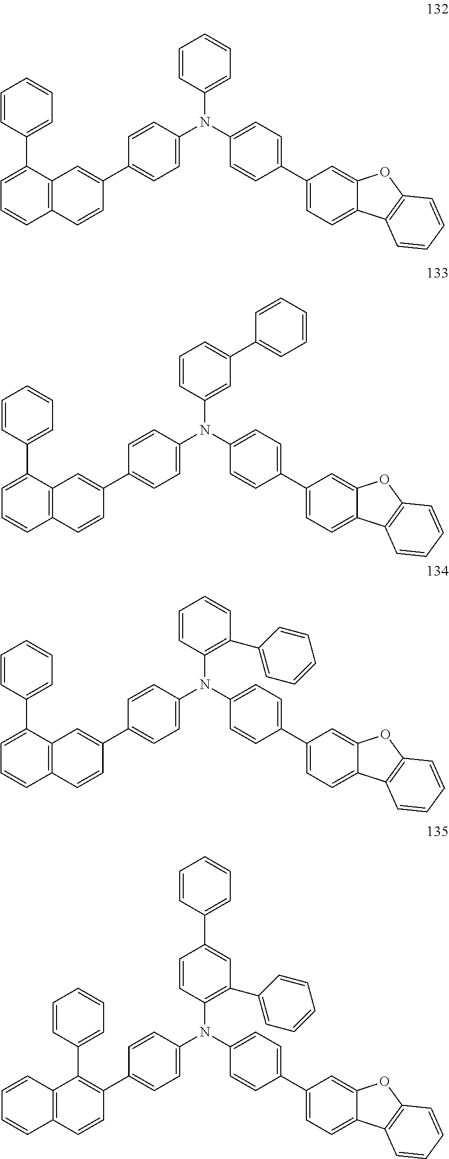 [SMILES: c1ccc(-c2cccc3ccc(-c4ccc(N(c5ccccc5)c5ccc(-c6ccc7c(c6)oc6ccccc67)cc5)cc4)cc23)cc1 ; c1ccc(-c2cccc(N(c3ccc(-c4ccc5c(c4)oc4ccccc45)cc3)c3ccc(-c4ccc5cccc(-c6ccccc6)c5c4)cc3)c2)cc1 ; c1ccc(-c2ccccc2N(c2ccc(-c3ccc4c(c3)oc3ccccc34)cc2)c2ccc(-c3ccc4cccc(-c5ccccc5)c4c3)cc2)cc1 ; c1ccc(-c2ccc(N(c3ccc(-c4ccc5c(c4)oc4ccccc45)cc3)c3ccc(-c4ccc5ccccc5c4-c4ccccc4)cc3)c(-c3ccccc3)c2)cc1] 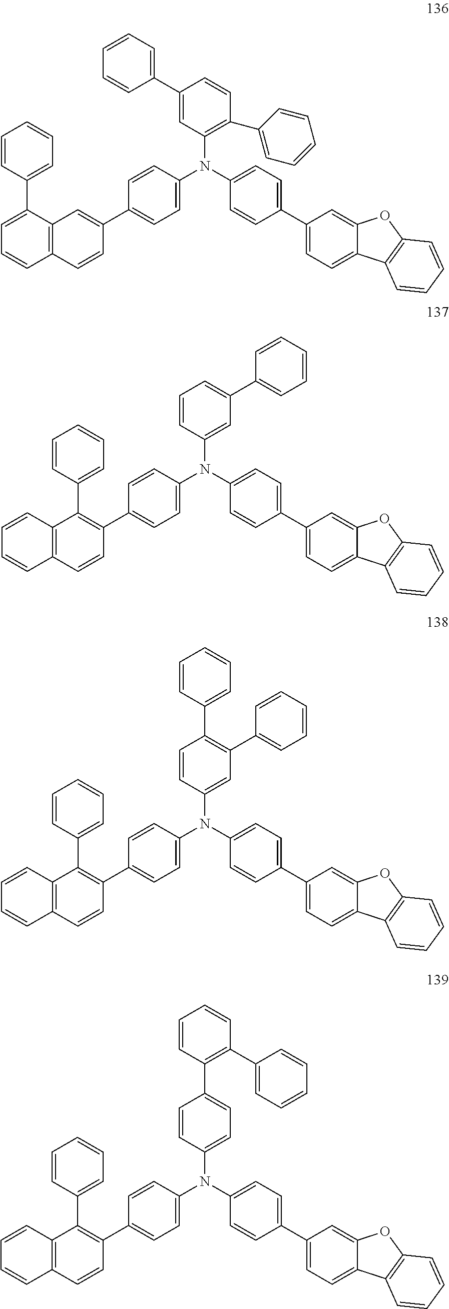 [SMILES: c1ccc(-c2ccc(-c3ccccc3)c(N(c3ccc(-c4ccc5c(c4)oc4ccccc45)cc3)c3ccc(-c4ccc5cccc(-c6ccccc6)c5c4)cc3)c2)cc1 ; c1ccc(-c2cccc(N(c3ccc(-c4ccc5c(c4)oc4ccccc45)cc3)c3ccc(-c4ccc5ccccc5c4-c4ccccc4)cc3)c2)cc1 ; c1ccc(-c2ccc(N(c3ccc(-c4ccc5c(c4)oc4ccccc45)cc3)c3ccc(-c4ccc5ccccc5c4-c4ccccc4)cc3)cc2-c2ccccc2)cc1 ; c1ccc(-c2ccccc2-c2ccc(N(c3ccc(-c4ccc5c(c4)oc4ccccc45)cc3)c3ccc(-c4ccc5ccccc5c4-c4ccccc4)cc3)cc2)cc1] 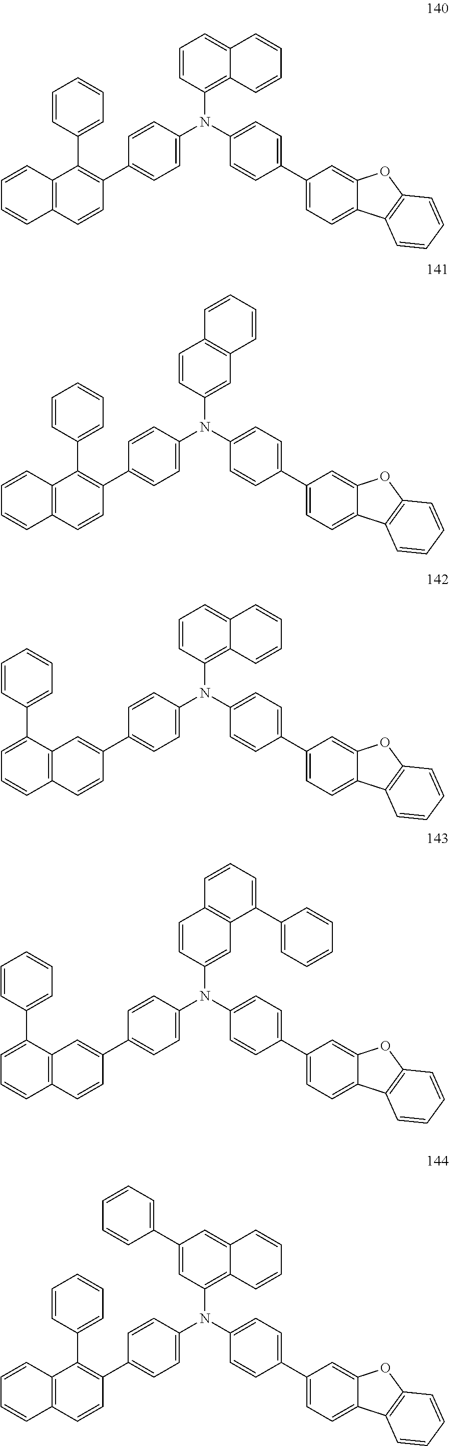 [SMILES: c1ccc(-c2c(-c3ccc(N(c4ccc(-c5ccc6c(c5)oc5ccccc56)cc4)c4cccc5ccccc45)cc3)ccc3ccccc23)cc1 ; c1ccc(-c2c(-c3ccc(N(c4ccc(-c5ccc6c(c5)oc5ccccc56)cc4)c4ccc5ccccc5c4)cc3)ccc3ccccc23)cc1 ; c1ccc(-c2cccc3ccc(-c4ccc(N(c5ccc(-c6ccc7c(c6)oc6ccccc67)cc5)c5cccc6ccccc56)cc4)cc23)cc1 ; c1ccc(-c2cccc3ccc(-c4ccc(N(c5ccc(-c6ccc7c(c6)oc6ccccc67)cc5)c5ccc6cccc(-c7ccccc7)c6c5)cc4)cc23)cc1 ; c1ccc(-c2cc(N(c3ccc(-c4ccc5c(c4)oc4ccccc45)cc3)c3ccc(-c4ccc5ccccc5c4-c4ccccc4)cc3)c3ccccc3c2)cc1] 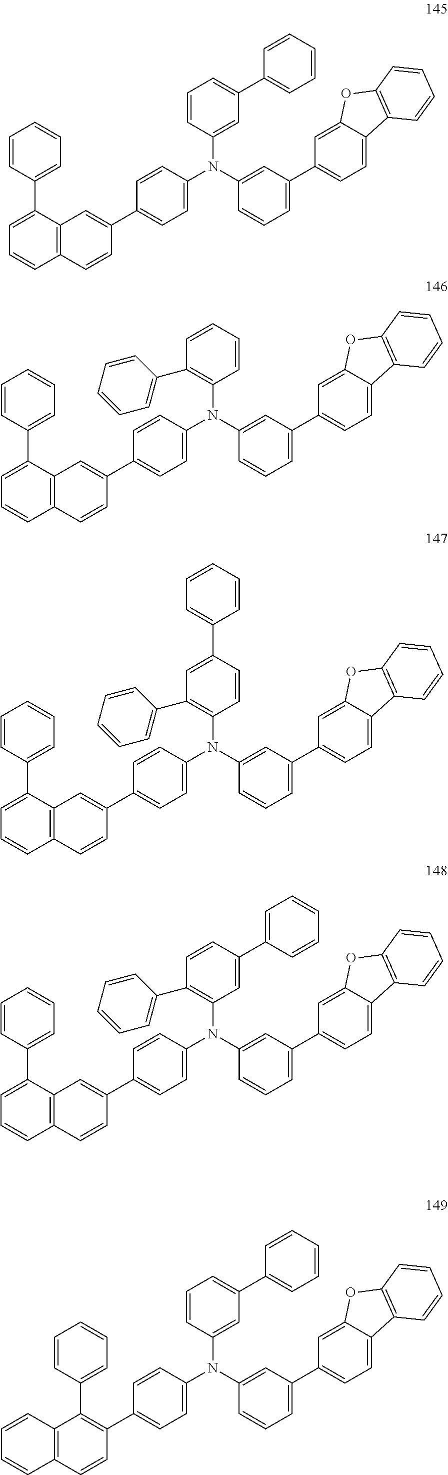 [SMILES: c1ccc(-c2cccc(N(c3ccc(-c4ccc5cccc(-c6ccccc6)c5c4)cc3)c3cccc(-c4ccc5c(c4)oc4ccccc45)c3)c2)cc1 ; c1ccc(-c2ccccc2N(c2ccc(-c3ccc4cccc(-c5ccccc5)c4c3)cc2)c2cccc(-c3ccc4c(c3)oc3ccccc34)c2)cc1 ; c1ccc(-c2ccc(N(c3ccc(-c4ccc5cccc(-c6ccccc6)c5c4)cc3)c3cccc(-c4ccc5c(c4)oc4ccccc45)c3)c(-c3ccccc3)c2)cc1 ; c1ccc(-c2ccc(-c3ccccc3)c(N(c3ccc(-c4ccc5cccc(-c6ccccc6)c5c4)cc3)c3cccc(-c4ccc5c(c4)oc4ccccc45)c3)c2)cc1 ; c1ccc(-c2cccc(N(c3ccc(-c4ccc5ccccc5c4-c4ccccc4)cc3)c3cccc(-c4ccc5c(c4)oc4ccccc45)c3)c2)cc1] 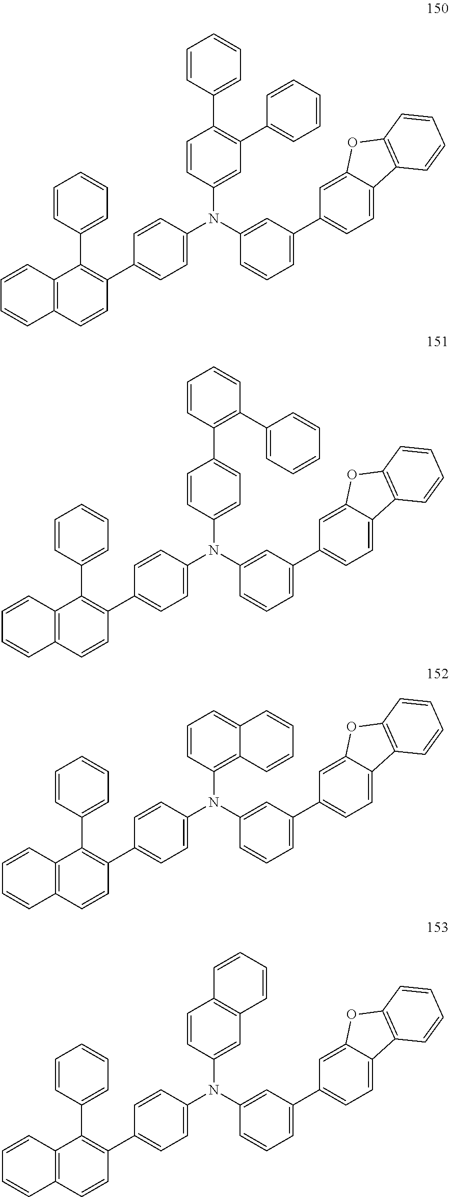 [SMILES: c1ccc(-c2ccc(N(c3ccc(-c4ccc5ccccc5c4-c4ccccc4)cc3)c3cccc(-c4ccc5c(c4)oc4ccccc45)c3)cc2-c2ccccc2)cc1 ; c1ccc(-c2ccccc2-c2ccc(N(c3ccc(-c4ccc5ccccc5c4-c4ccccc4)cc3)c3cccc(-c4ccc5c(c4)oc4ccccc45)c3)cc2)cc1 ; c1ccc(-c2c(-c3ccc(N(c4cccc(-c5ccc6c(c5)oc5ccccc56)c4)c4cccc5ccccc45)cc3)ccc3ccccc23)cc1 ; c1ccc(-c2c(-c3ccc(N(c4cccc(-c5ccc6c(c5)oc5ccccc56)c4)c4ccc5ccccc5c4)cc3)ccc3ccccc23)cc1] 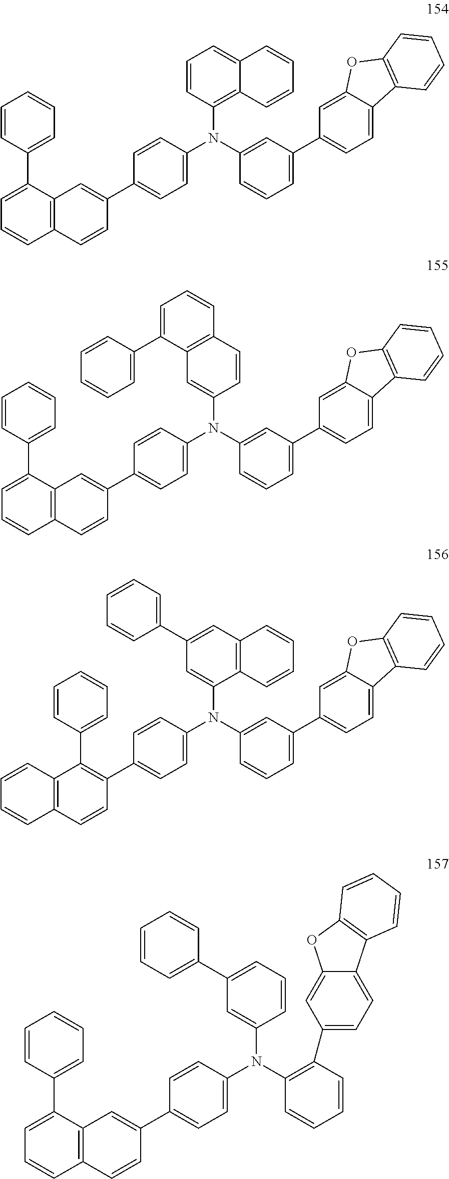 [SMILES: c1ccc(-c2cccc3ccc(-c4ccc(N(c5cccc(-c6ccc7c(c6)oc6ccccc67)c5)c5cccc6ccccc56)cc4)cc23)cc1 ; c1ccc(-c2cccc3ccc(-c4ccc(N(c5cccc(-c6ccc7c(c6)oc6ccccc67)c5)c5ccc6cccc(-c7ccccc7)c6c5)cc4)cc23)cc1 ; c1ccc(-c2cc(N(c3ccc(-c4ccc5ccccc5c4-c4ccccc4)cc3)c3cccc(-c4ccc5c(c4)oc4ccccc45)c3)c3ccccc3c2)cc1 ; c1ccc(-c2cccc(N(c3ccc(-c4ccc5cccc(-c6ccccc6)c5c4)cc3)c3ccccc3-c3ccc4c(c3)oc3ccccc34)c2)cc1] 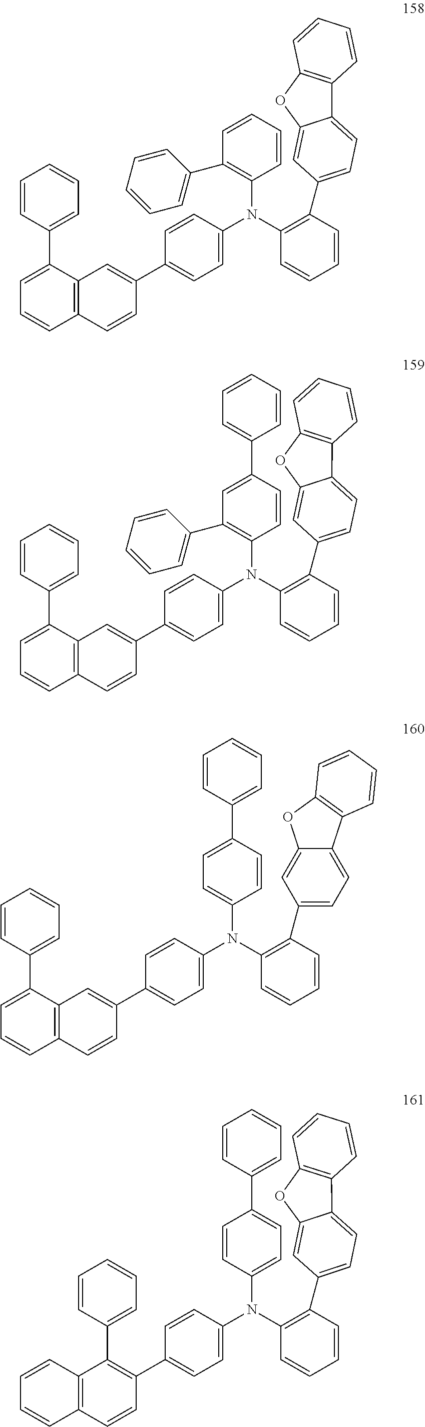 [SMILES: c1ccc(-c2ccccc2N(c2ccc(-c3ccc4cccc(-c5ccccc5)c4c3)cc2)c2ccccc2-c2ccc3c(c2)oc2ccccc23)cc1 ; c1ccc(-c2ccc(N(c3ccc(-c4ccc5cccc(-c6ccccc6)c5c4)cc3)c3ccccc3-c3ccc4c(c3)oc3ccccc34)c(-c3ccccc3)c2)cc1 ; c1ccc(-c2ccc(N(c3ccc(-c4ccc5cccc(-c6ccccc6)c5c4)cc3)c3ccccc3-c3ccc4c(c3)oc3ccccc34)cc2)cc1 ; c1ccc(-c2ccc(N(c3ccc(-c4ccc5ccccc5c4-c4ccccc4)cc3)c3ccccc3-c3ccc4c(c3)oc3ccccc34)cc2)cc1] 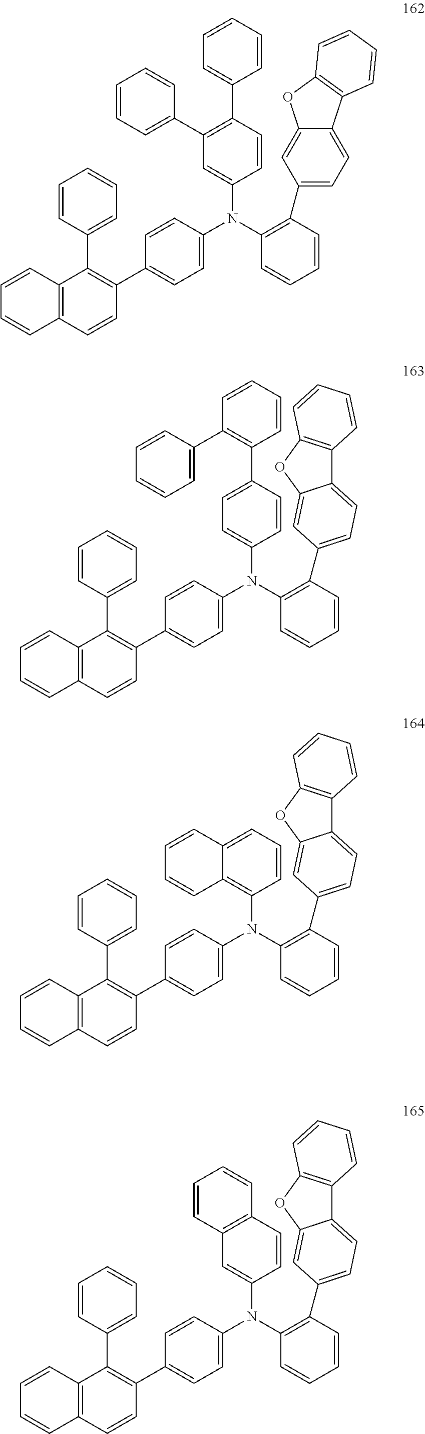 [SMILES: c1ccc(-c2ccc(N(c3ccc(-c4ccc5ccccc5c4-c4ccccc4)cc3)c3ccccc3-c3ccc4c(c3)oc3ccccc34)cc2-c2ccccc2)cc1 ; c1ccc(-c2ccccc2-c2ccc(N(c3ccc(-c4ccc5ccccc5c4-c4ccccc4)cc3)c3ccccc3-c3ccc4c(c3)oc3ccccc34)cc2)cc1 ; c1ccc(-c2c(-c3ccc(N(c4ccccc4-c4ccc5c(c4)oc4ccccc45)c4cccc5ccccc45)cc3)ccc3ccccc23)cc1 ; c1ccc(-c2c(-c3ccc(N(c4ccc5ccccc5c4)c4ccccc4-c4ccc5c(c4)oc4ccccc45)cc3)ccc3ccccc23)cc1] 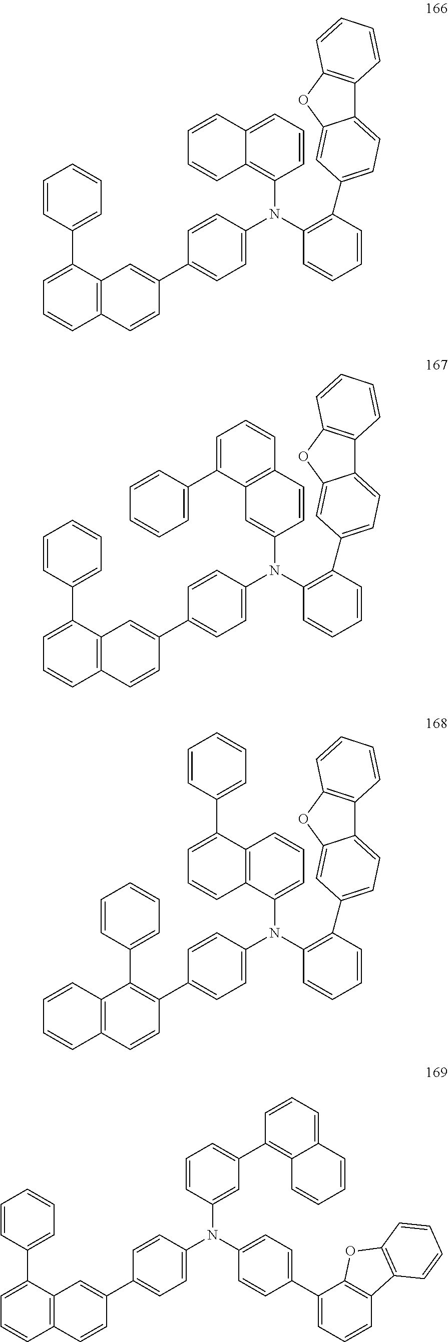 [SMILES: c1ccc(-c2cccc3ccc(-c4ccc(N(c5ccccc5-c5ccc6c(c5)oc5ccccc56)c5cccc6ccccc56)cc4)cc23)cc1 ; c1ccc(-c2cccc3ccc(-c4ccc(N(c5ccc6cccc(-c7ccccc7)c6c5)c5ccccc5-c5ccc6c(c5)oc5ccccc56)cc4)cc23)cc1 ; c1ccc(-c2c(-c3ccc(N(c4ccccc4-c4ccc5c(c4)oc4ccccc45)c4cccc5c(-c6ccccc6)cccc45)cc3)ccc3ccccc23)cc1 ; c1ccc(-c2cccc3ccc(-c4ccc(N(c5ccc(-c6cccc7c6oc6ccccc67)cc5)c5cccc(-c6cccc7ccccc67)c5)cc4)cc23)cc1] 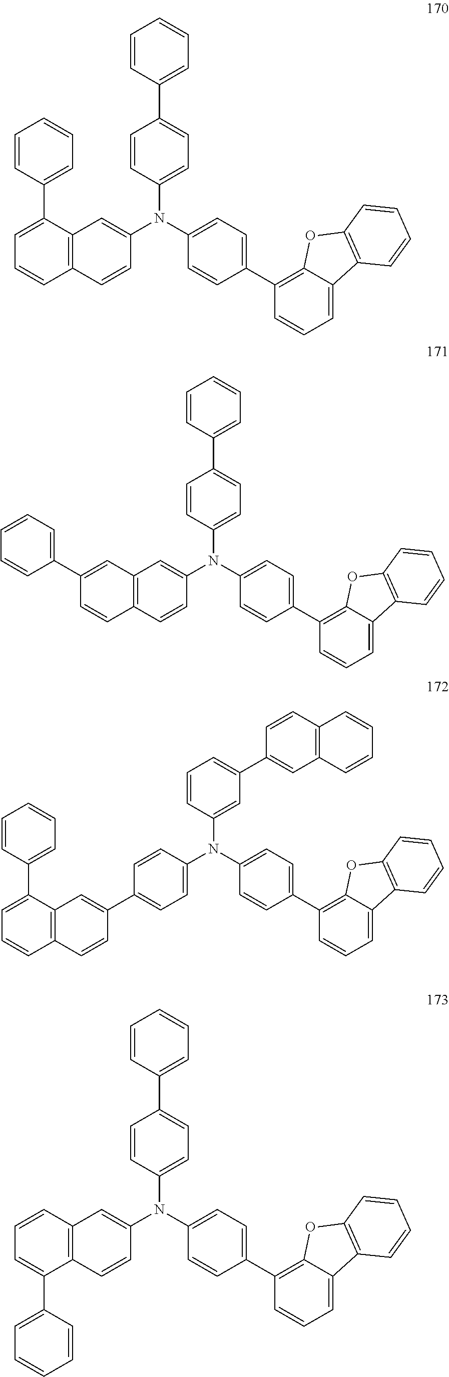 [SMILES: c1ccc(-c2ccc(N(c3ccc(-c4cccc5c4oc4ccccc45)cc3)c3ccc4cccc(-c5ccccc5)c4c3)cc2)cc1 ; c1ccc(-c2ccc(N(c3ccc(-c4cccc5c4oc4ccccc45)cc3)c3ccc4ccc(-c5ccccc5)cc4c3)cc2)cc1 ; c1ccc(-c2cccc3ccc(-c4ccc(N(c5ccc(-c6cccc7c6oc6ccccc67)cc5)c5cccc(-c6ccc7ccccc7c6)c5)cc4)cc23)cc1 ; c1ccc(-c2ccc(N(c3ccc(-c4cccc5c4oc4ccccc45)cc3)c3ccc4c(-c5ccccc5)cccc4c3)cc2)cc1] 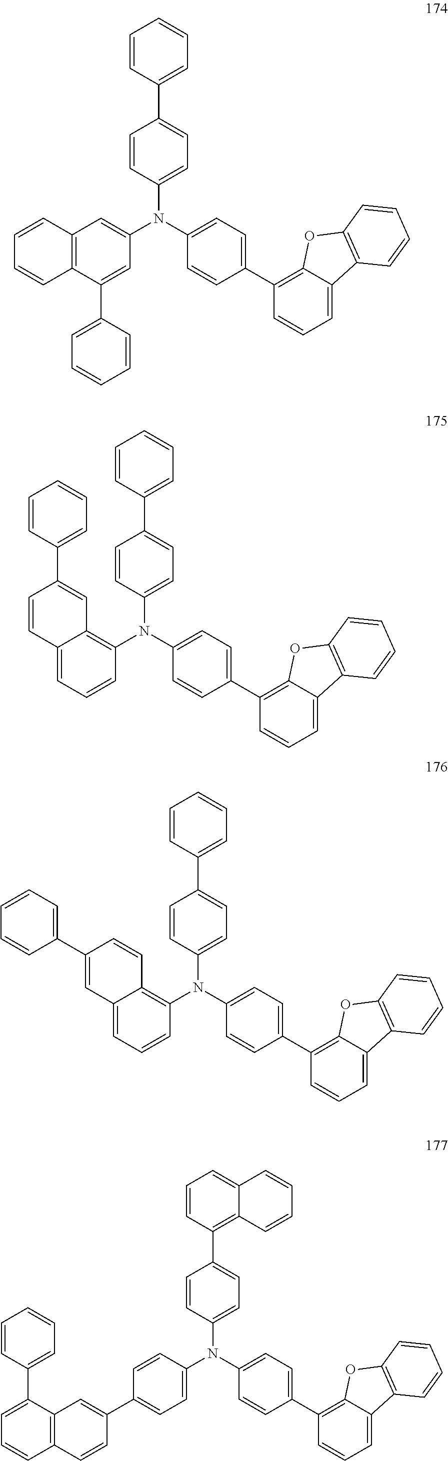 [SMILES: c1ccc(-c2ccc(N(c3ccc(-c4cccc5c4oc4ccccc45)cc3)c3cc(-c4ccccc4)c4ccccc4c3)cc2)cc1 ; c1ccc(-c2ccc(N(c3ccc(-c4cccc5c4oc4ccccc45)cc3)c3cccc4ccc(-c5ccccc5)cc34)cc2)cc1 ; c1ccc(-c2ccc(N(c3ccc(-c4cccc5c4oc4ccccc45)cc3)c3cccc4cc(-c5ccccc5)ccc34)cc2)cc1 ; c1ccc(-c2cccc3ccc(-c4ccc(N(c5ccc(-c6cccc7ccccc67)cc5)c5ccc(-c6cccc7c6oc6ccccc67)cc5)cc4)cc23)cc1] 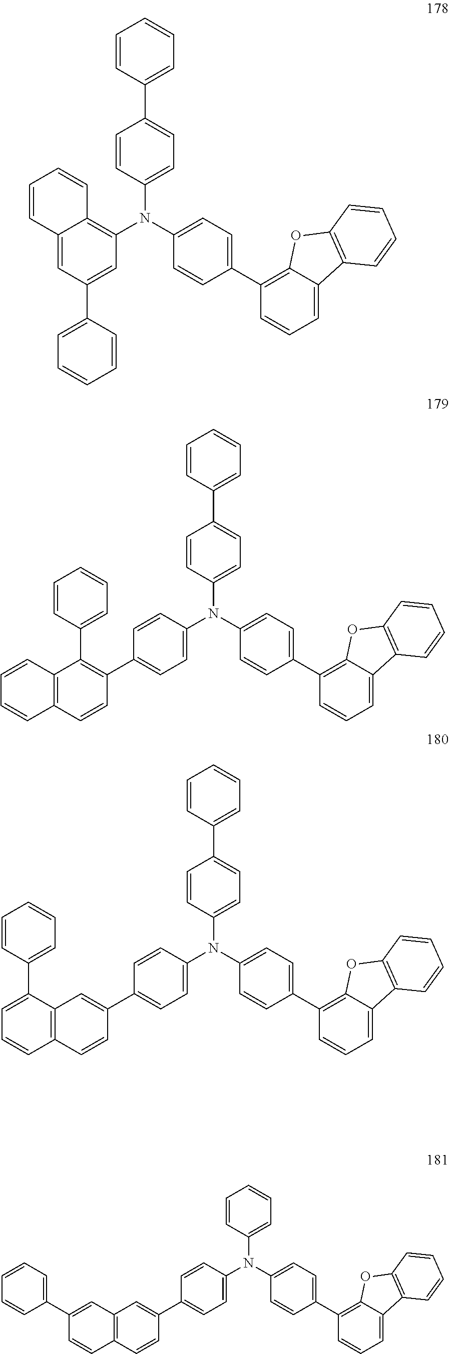 [SMILES: c1ccc(-c2ccc(N(c3ccc(-c4cccc5c4oc4ccccc45)cc3)c3cc(-c4ccccc4)cc4ccccc34)cc2)cc1 ; c1ccc(-c2ccc(N(c3ccc(-c4ccc5ccccc5c4-c4ccccc4)cc3)c3ccc(-c4cccc5c4oc4ccccc45)cc3)cc2)cc1 ; c1ccc(-c2ccc(N(c3ccc(-c4ccc5cccc(-c6ccccc6)c5c4)cc3)c3ccc(-c4cccc5c4oc4ccccc45)cc3)cc2)cc1 ; c1ccc(-c2ccc3ccc(-c4ccc(N(c5ccccc5)c5ccc(-c6cccc7c6oc6ccccc67)cc5)cc4)cc3c2)cc1] 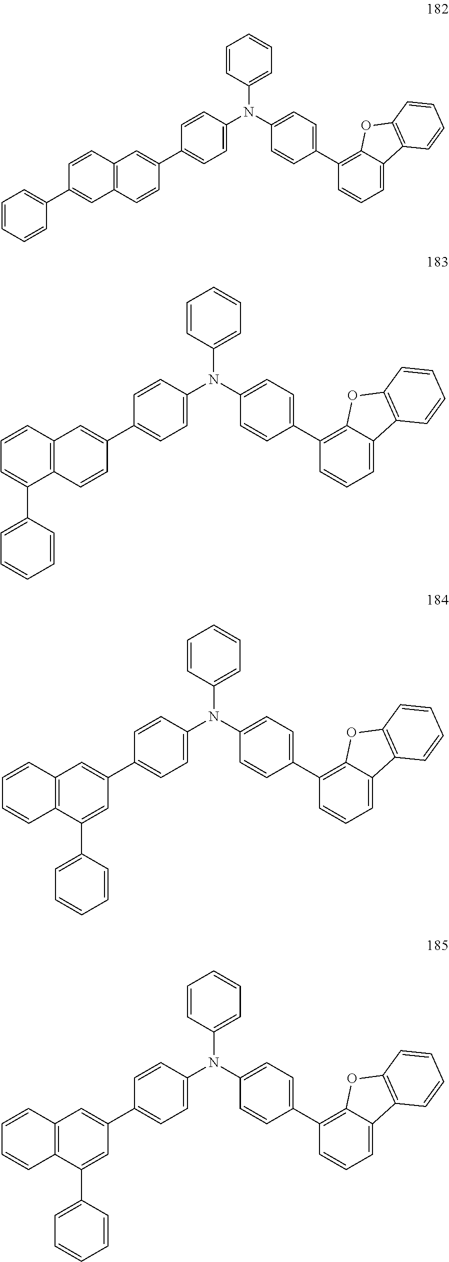 [SMILES: c1ccc(-c2ccc3cc(-c4ccc(N(c5ccccc5)c5ccc(-c6cccc7c6oc6ccccc67)cc5)cc4)ccc3c2)cc1 ; c1ccc(-c2cccc3cc(-c4ccc(N(c5ccccc5)c5ccc(-c6cccc7c6oc6ccccc67)cc5)cc4)ccc23)cc1 ; c1ccc(-c2cc(-c3ccc(N(c4ccccc4)c4ccc(-c5cccc6c5oc5ccccc56)cc4)cc3)cc3ccccc23)cc1 ; c1ccc(-c2cc(-c3ccc(N(c4ccccc4)c4ccc(-c5cccc6c5oc5ccccc56)cc4)cc3)cc3ccccc23)cc1] 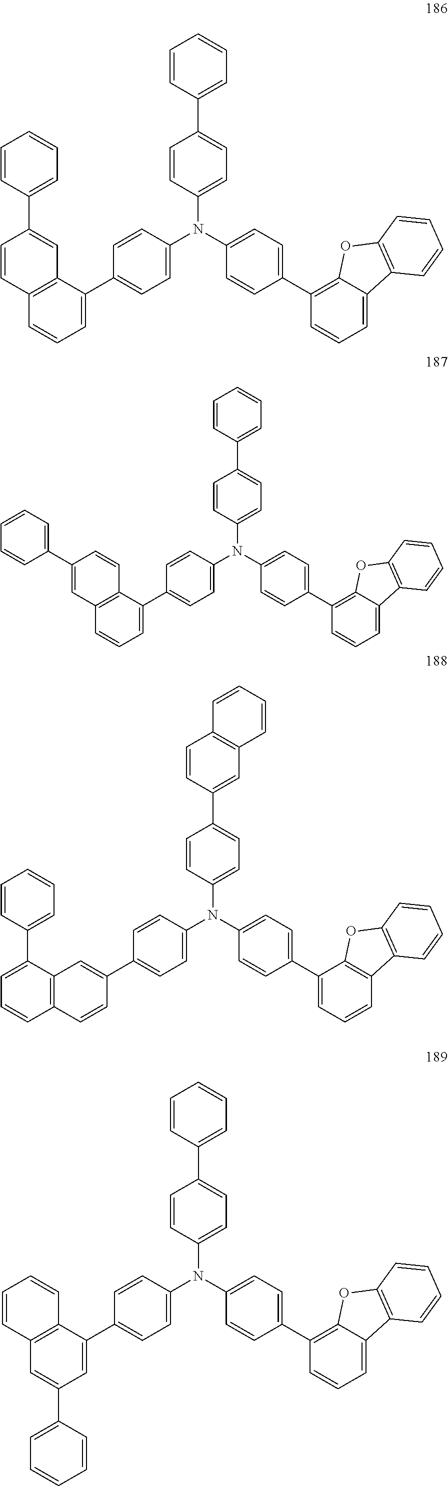 [SMILES: c1ccc(-c2ccc(N(c3ccc(-c4cccc5ccc(-c6ccccc6)cc45)cc3)c3ccc(-c4cccc5c4oc4ccccc45)cc3)cc2)cc1 ; c1ccc(-c2ccc(N(c3ccc(-c4cccc5cc(-c6ccccc6)ccc45)cc3)c3ccc(-c4cccc5c4oc4ccccc45)cc3)cc2)cc1 ; c1ccc(-c2cccc3ccc(-c4ccc(N(c5ccc(-c6ccc7ccccc7c6)cc5)c5ccc(-c6cccc7c6oc6ccccc67)cc5)cc4)cc23)cc1 ; c1ccc(-c2ccc(N(c3ccc(-c4cc(-c5ccccc5)cc5ccccc45)cc3)c3ccc(-c4cccc5c4oc4ccccc45)cc3)cc2)cc1] 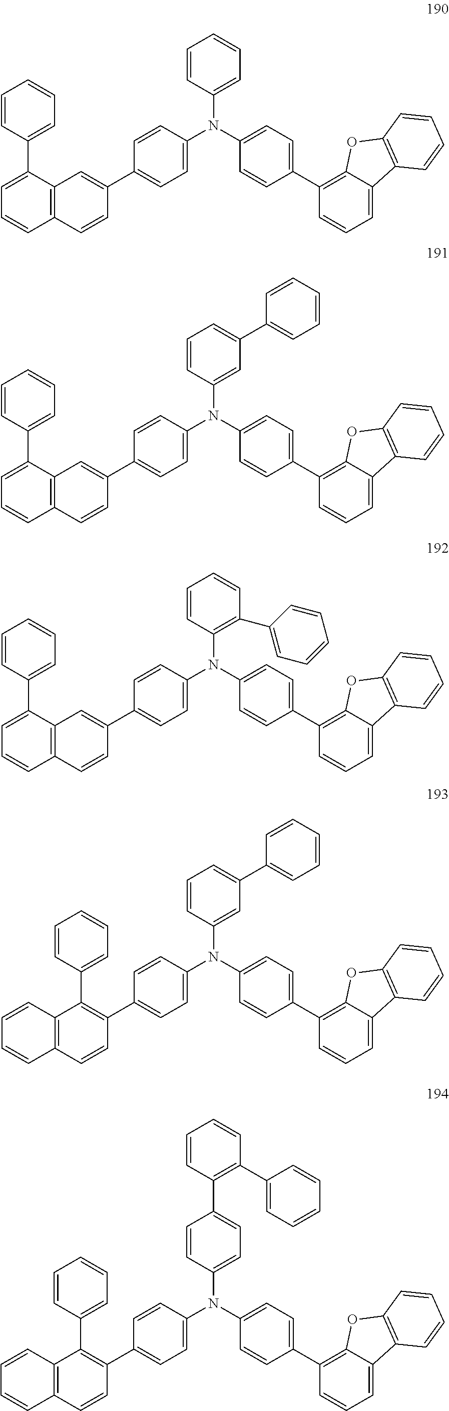 [SMILES: c1ccc(-c2cccc3ccc(-c4ccc(N(c5ccccc5)c5ccc(-c6cccc7c6oc6ccccc67)cc5)cc4)cc23)cc1 ; c1ccc(-c2cccc(N(c3ccc(-c4ccc5cccc(-c6ccccc6)c5c4)cc3)c3ccc(-c4cccc5c4oc4ccccc45)cc3)c2)cc1 ; c1ccc(-c2ccccc2N(c2ccc(-c3ccc4cccc(-c5ccccc5)c4c3)cc2)c2ccc(-c3cccc4c3oc3ccccc34)cc2)cc1 ; c1ccc(-c2cccc(N(c3ccc(-c4ccc5ccccc5c4-c4ccccc4)cc3)c3ccc(-c4cccc5c4oc4ccccc45)cc3)c2)cc1 ; c1ccc(-c2ccccc2-c2ccc(N(c3ccc(-c4ccc5ccccc5c4-c4ccccc4)cc3)c3ccc(-c4cccc5c4oc4ccccc45)cc3)cc2)cc1] 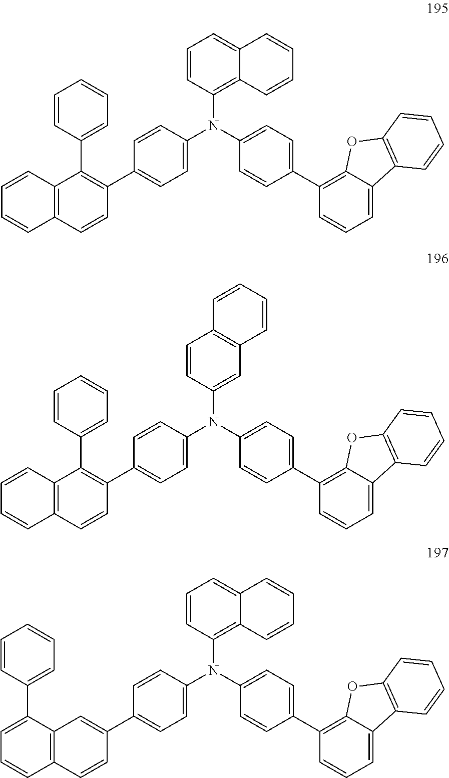 [SMILES: c1ccc(-c2c(-c3ccc(N(c4ccc(-c5cccc6c5oc5ccccc56)cc4)c4cccc5ccccc45)cc3)ccc3ccccc23)cc1 ; c1ccc(-c2c(-c3ccc(N(c4ccc(-c5cccc6c5oc5ccccc56)cc4)c4ccc5ccccc5c4)cc3)ccc3ccccc23)cc1 ; c1ccc(-c2cccc3ccc(-c4ccc(N(c5ccc(-c6cccc7c6oc6ccccc67)cc5)c5cccc6ccccc56)cc4)cc23)cc1] 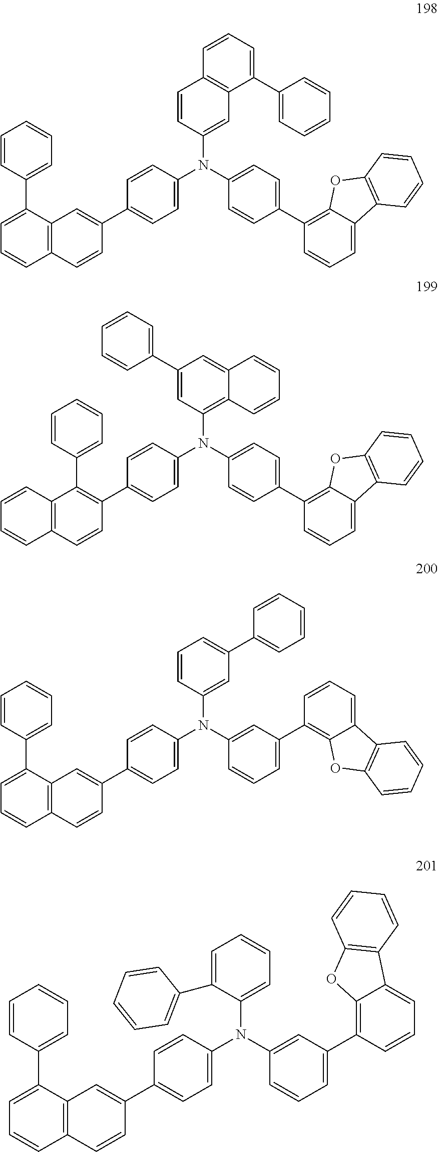 [SMILES: c1ccc(-c2cccc3ccc(-c4ccc(N(c5ccc(-c6cccc7c6oc6ccccc67)cc5)c5ccc6cccc(-c7ccccc7)c6c5)cc4)cc23)cc1 ; c1ccc(-c2cc(N(c3ccc(-c4ccc5ccccc5c4-c4ccccc4)cc3)c3ccc(-c4cccc5c4oc4ccccc45)cc3)c3ccccc3c2)cc1 ; c1ccc(-c2cccc(N(c3ccc(-c4ccc5cccc(-c6ccccc6)c5c4)cc3)c3cccc(-c4cccc5c4oc4ccccc45)c3)c2)cc1 ; c1ccc(-c2ccccc2N(c2ccc(-c3ccc4cccc(-c5ccccc5)c4c3)cc2)c2cccc(-c3cccc4c3oc3ccccc34)c2)cc1] 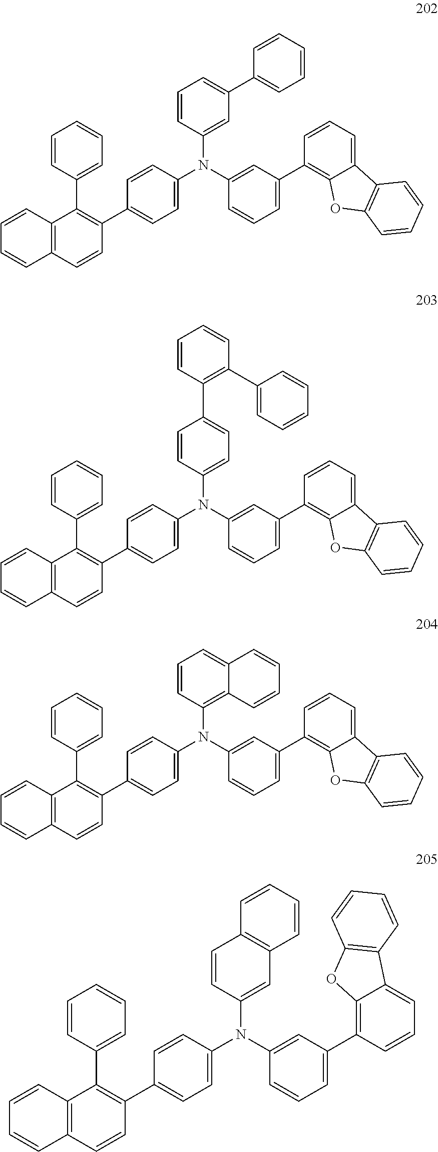 [SMILES: c1ccc(-c2cccc(N(c3ccc(-c4ccc5ccccc5c4-c4ccccc4)cc3)c3cccc(-c4cccc5c4oc4ccccc45)c3)c2)cc1 ; c1ccc(-c2ccccc2-c2ccc(N(c3ccc(-c4ccc5ccccc5c4-c4ccccc4)cc3)c3cccc(-c4cccc5c4oc4ccccc45)c3)cc2)cc1 ; c1ccc(-c2c(-c3ccc(N(c4cccc(-c5cccc6c5oc5ccccc56)c4)c4cccc5ccccc45)cc3)ccc3ccccc23)cc1 ; c1ccc(-c2c(-c3ccc(N(c4cccc(-c5cccc6c5oc5ccccc56)c4)c4ccc5ccccc5c4)cc3)ccc3ccccc23)cc1] 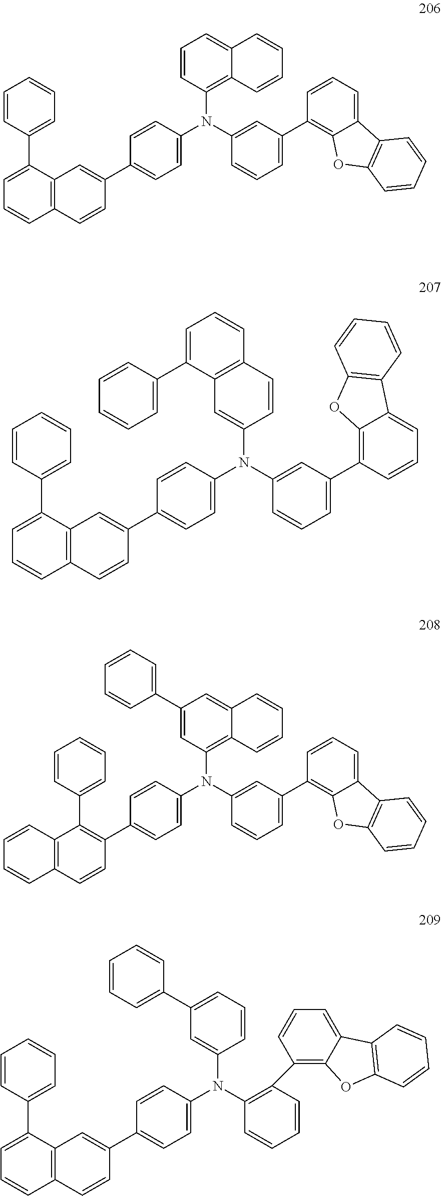 [SMILES: c1ccc(-c2cccc3ccc(-c4ccc(N(c5cccc(-c6cccc7c6oc6ccccc67)c5)c5cccc6ccccc56)cc4)cc23)cc1 ; c1ccc(-c2cccc3ccc(-c4ccc(N(c5cccc(-c6cccc7c6oc6ccccc67)c5)c5ccc6cccc(-c7ccccc7)c6c5)cc4)cc23)cc1 ; c1ccc(-c2cc(N(c3ccc(-c4ccc5ccccc5c4-c4ccccc4)cc3)c3cccc(-c4cccc5c4oc4ccccc45)c3)c3ccccc3c2)cc1 ; c1ccc(-c2cccc(N(c3ccc(-c4ccc5cccc(-c6ccccc6)c5c4)cc3)c3ccccc3-c3cccc4c3oc3ccccc34)c2)cc1] 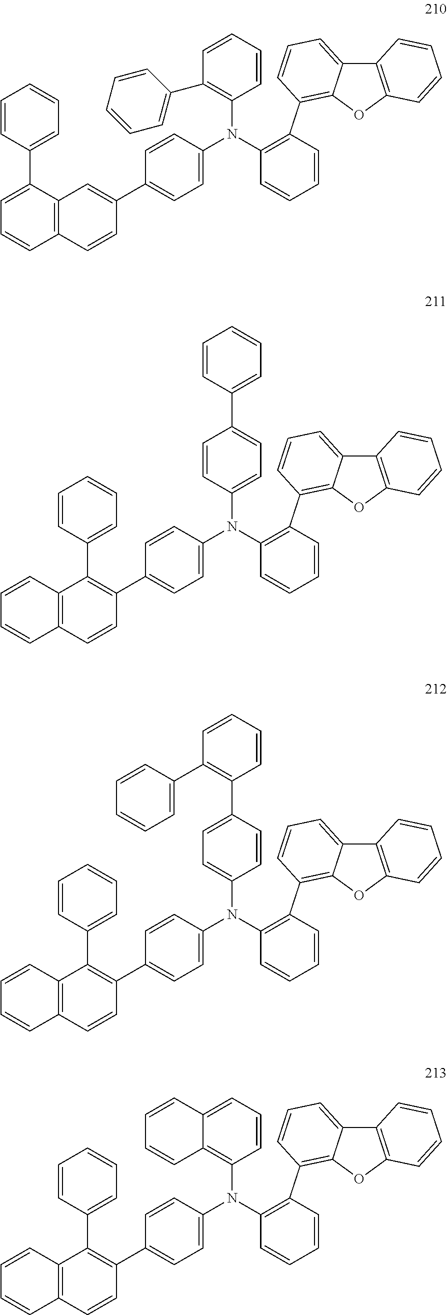 [SMILES: c1ccc(-c2ccccc2N(c2ccc(-c3ccc4cccc(-c5ccccc5)c4c3)cc2)c2ccccc2-c2cccc3c2oc2ccccc23)cc1 ; c1ccc(-c2ccc(N(c3ccc(-c4ccc5ccccc5c4-c4ccccc4)cc3)c3ccccc3-c3cccc4c3oc3ccccc34)cc2)cc1 ; c1ccc(-c2ccccc2-c2ccc(N(c3ccc(-c4ccc5ccccc5c4-c4ccccc4)cc3)c3ccccc3-c3cccc4c3oc3ccccc34)cc2)cc1 ; c1ccc(-c2c(-c3ccc(N(c4ccccc4-c4cccc5c4oc4ccccc45)c4cccc5ccccc45)cc3)ccc3ccccc23)cc1] 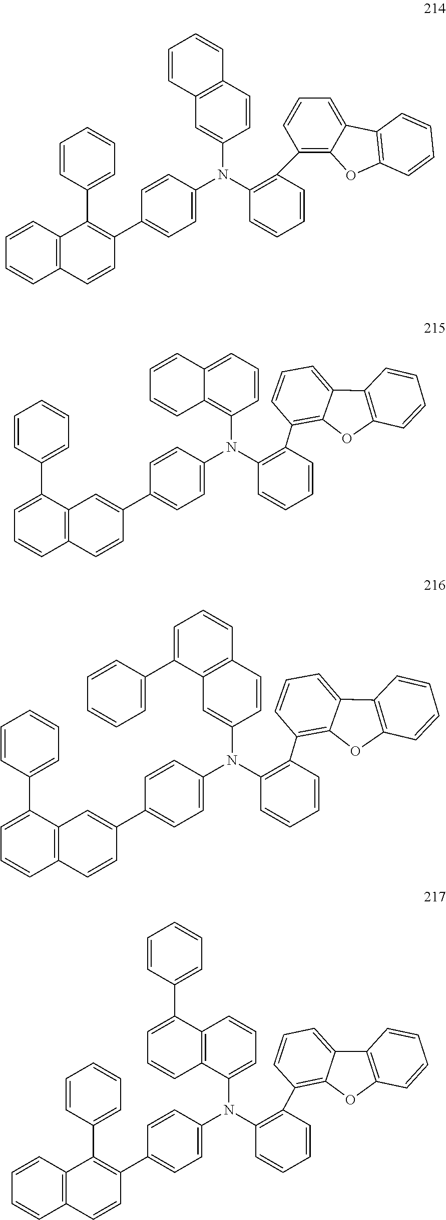 [SMILES: c1ccc(-c2c(-c3ccc(N(c4ccc5ccccc5c4)c4ccccc4-c4cccc5c4oc4ccccc45)cc3)ccc3ccccc23)cc1 ; c1ccc(-c2cccc3ccc(-c4ccc(N(c5ccccc5-c5cccc6c5oc5ccccc56)c5cccc6ccccc56)cc4)cc23)cc1 ; c1ccc(-c2cccc3ccc(-c4ccc(N(c5ccc6cccc(-c7ccccc7)c6c5)c5ccccc5-c5cccc6c5oc5ccccc56)cc4)cc23)cc1 ; c1ccc(-c2c(-c3ccc(N(c4ccccc4-c4cccc5c4oc4ccccc45)c4cccc5c(-c6ccccc6)cccc45)cc3)ccc3ccccc23)cc1] 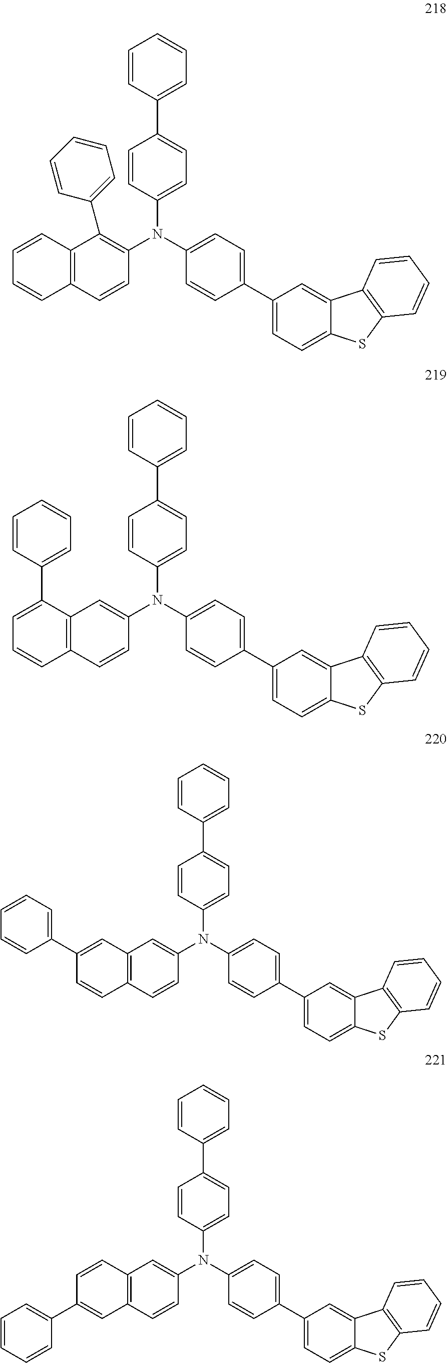 [SMILES: c1ccc(-c2ccc(N(c3ccc(-c4ccc5sc6ccccc6c5c4)cc3)c3ccc4ccccc4c3-c3ccccc3)cc2)cc1 ; c1ccc(-c2ccc(N(c3ccc(-c4ccc5sc6ccccc6c5c4)cc3)c3ccc4cccc(-c5ccccc5)c4c3)cc2)cc1 ; c1ccc(-c2ccc(N(c3ccc(-c4ccc5sc6ccccc6c5c4)cc3)c3ccc4ccc(-c5ccccc5)cc4c3)cc2)cc1 ; c1ccc(-c2ccc(N(c3ccc(-c4ccc5sc6ccccc6c5c4)cc3)c3ccc4cc(-c5ccccc5)ccc4c3)cc2)cc1] 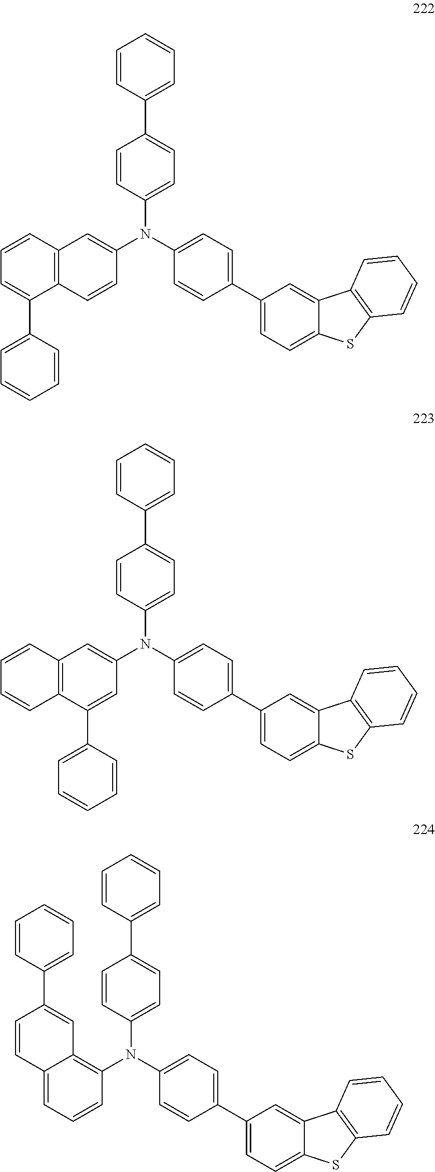 [SMILES: c1ccc(-c2ccc(N(c3ccc(-c4ccc5sc6ccccc6c5c4)cc3)c3ccc4c(-c5ccccc5)cccc4c3)cc2)cc1 ; c1ccc(-c2ccc(N(c3ccc(-c4ccc5sc6ccccc6c5c4)cc3)c3cc(-c4ccccc4)c4ccccc4c3)cc2)cc1 ; c1ccc(-c2ccc(N(c3ccc(-c4ccc5sc6ccccc6c5c4)cc3)c3cccc4ccc(-c5ccccc5)cc34)cc2)cc1] 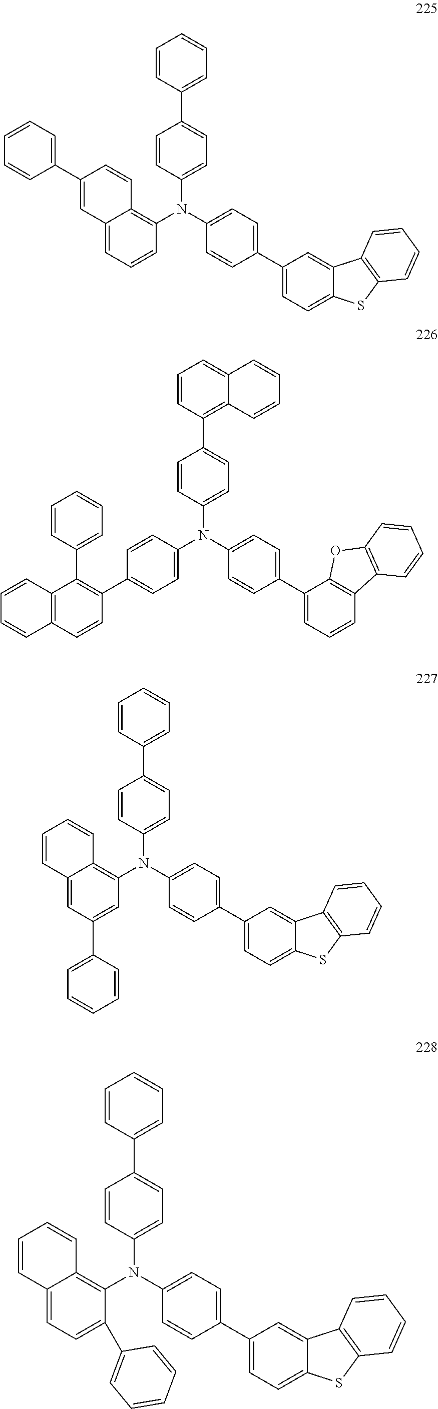 [SMILES: c1ccc(-c2ccc(N(c3ccc(-c4ccc5sc6ccccc6c5c4)cc3)c3cccc4cc(-c5ccccc5)ccc34)cc2)cc1 ; c1ccc(-c2c(-c3ccc(N(c4ccc(-c5cccc6ccccc56)cc4)c4ccc(-c5cccc6c5oc5ccccc56)cc4)cc3)ccc3ccccc23)cc1 ; c1ccc(-c2ccc(N(c3ccc(-c4ccc5sc6ccccc6c5c4)cc3)c3cc(-c4ccccc4)cc4ccccc34)cc2)cc1 ; c1ccc(-c2ccc(N(c3ccc(-c4ccc5sc6ccccc6c5c4)cc3)c3c(-c4ccccc4)ccc4ccccc34)cc2)cc1] 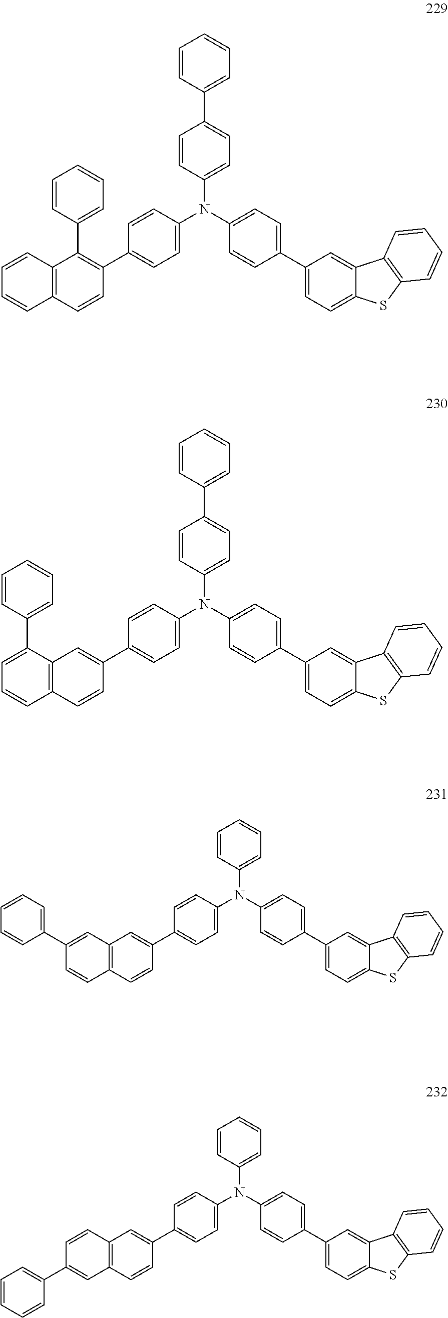 [SMILES: c1ccc(-c2ccc(N(c3ccc(-c4ccc5sc6ccccc6c5c4)cc3)c3ccc(-c4ccc5ccccc5c4-c4ccccc4)cc3)cc2)cc1 ; c1ccc(-c2ccc(N(c3ccc(-c4ccc5cccc(-c6ccccc6)c5c4)cc3)c3ccc(-c4ccc5sc6ccccc6c5c4)cc3)cc2)cc1 ; c1ccc(-c2ccc3ccc(-c4ccc(N(c5ccccc5)c5ccc(-c6ccc7sc8ccccc8c7c6)cc5)cc4)cc3c2)cc1 ; c1ccc(-c2ccc3cc(-c4ccc(N(c5ccccc5)c5ccc(-c6ccc7sc8ccccc8c7c6)cc5)cc4)ccc3c2)cc1] 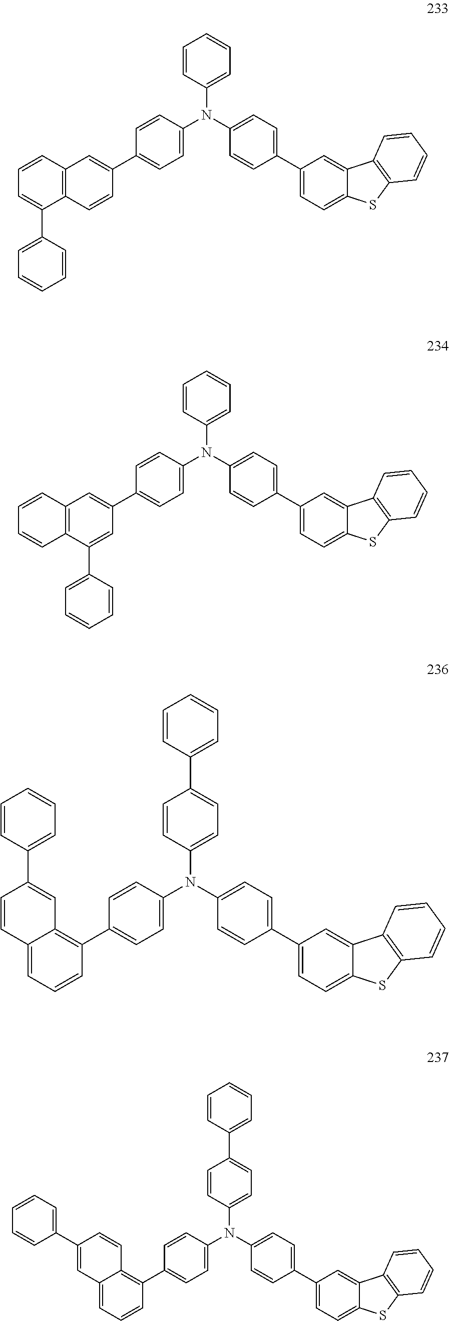 [SMILES: c1ccc(-c2cccc3cc(-c4ccc(N(c5ccccc5)c5ccc(-c6ccc7sc8ccccc8c7c6)cc5)cc4)ccc23)cc1 ; c1ccc(-c2cc(-c3ccc(N(c4ccccc4)c4ccc(-c5ccc6sc7ccccc7c6c5)cc4)cc3)cc3ccccc23)cc1 ; c1ccc(-c2ccc(N(c3ccc(-c4ccc5sc6ccccc6c5c4)cc3)c3ccc(-c4cccc5ccc(-c6ccccc6)cc45)cc3)cc2)cc1 ; c1ccc(-c2ccc(N(c3ccc(-c4ccc5sc6ccccc6c5c4)cc3)c3ccc(-c4cccc5cc(-c6ccccc6)ccc45)cc3)cc2)cc1] 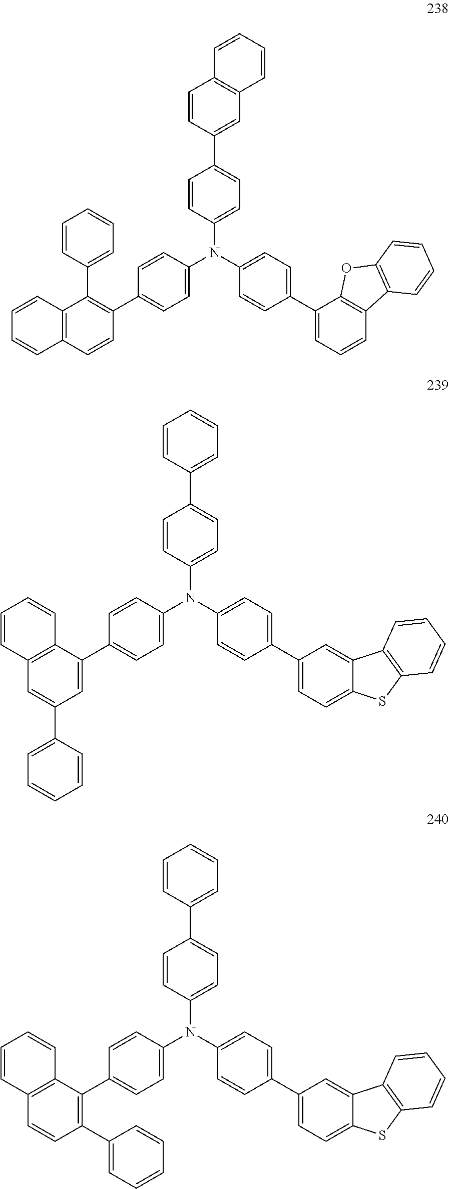 [SMILES: c1ccc(-c2c(-c3ccc(N(c4ccc(-c5ccc6ccccc6c5)cc4)c4ccc(-c5cccc6c5oc5ccccc56)cc4)cc3)ccc3ccccc23)cc1 ; c1ccc(-c2ccc(N(c3ccc(-c4ccc5sc6ccccc6c5c4)cc3)c3ccc(-c4cc(-c5ccccc5)cc5ccccc45)cc3)cc2)cc1 ; c1ccc(-c2ccc(N(c3ccc(-c4ccc5sc6ccccc6c5c4)cc3)c3ccc(-c4c(-c5ccccc5)ccc5ccccc45)cc3)cc2)cc1] 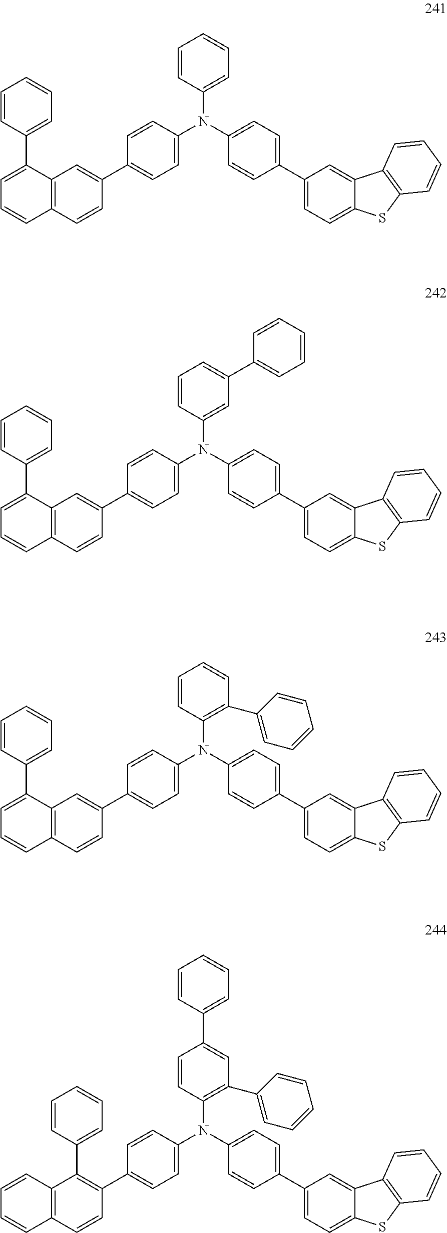 [SMILES: c1ccc(-c2cccc3ccc(-c4ccc(N(c5ccccc5)c5ccc(-c6ccc7sc8ccccc8c7c6)cc5)cc4)cc23)cc1 ; c1ccc(-c2cccc(N(c3ccc(-c4ccc5cccc(-c6ccccc6)c5c4)cc3)c3ccc(-c4ccc5sc6ccccc6c5c4)cc3)c2)cc1 ; c1ccc(-c2ccccc2N(c2ccc(-c3ccc4cccc(-c5ccccc5)c4c3)cc2)c2ccc(-c3ccc4sc5ccccc5c4c3)cc2)cc1 ; c1ccc(-c2ccc(N(c3ccc(-c4ccc5sc6ccccc6c5c4)cc3)c3ccc(-c4ccc5ccccc5c4-c4ccccc4)cc3)c(-c3ccccc3)c2)cc1] 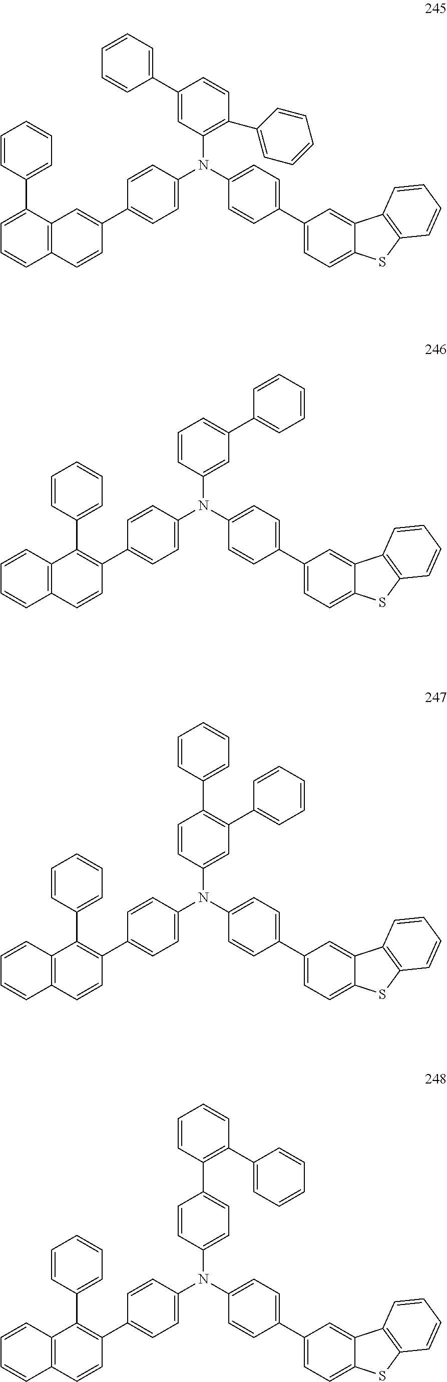 [SMILES: c1ccc(-c2ccc(-c3ccccc3)c(N(c3ccc(-c4ccc5cccc(-c6ccccc6)c5c4)cc3)c3ccc(-c4ccc5sc6ccccc6c5c4)cc3)c2)cc1 ; c1ccc(-c2cccc(N(c3ccc(-c4ccc5sc6ccccc6c5c4)cc3)c3ccc(-c4ccc5ccccc5c4-c4ccccc4)cc3)c2)cc1 ; c1ccc(-c2ccc(N(c3ccc(-c4ccc5sc6ccccc6c5c4)cc3)c3ccc(-c4ccc5ccccc5c4-c4ccccc4)cc3)cc2-c2ccccc2)cc1 ; c1ccc(-c2ccccc2-c2ccc(N(c3ccc(-c4ccc5sc6ccccc6c5c4)cc3)c3ccc(-c4ccc5ccccc5c4-c4ccccc4)cc3)cc2)cc1] 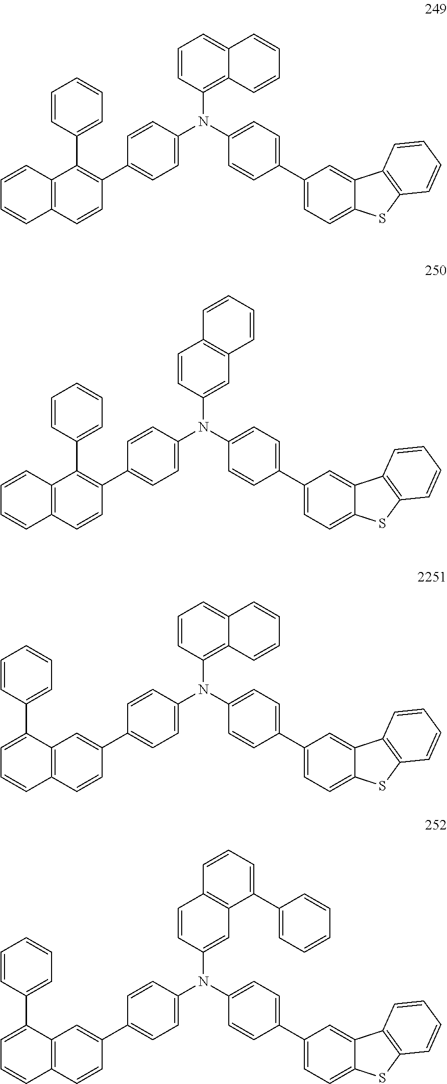 [SMILES: c1ccc(-c2c(-c3ccc(N(c4ccc(-c5ccc6sc7ccccc7c6c5)cc4)c4cccc5ccccc45)cc3)ccc3ccccc23)cc1 ; c1ccc(-c2c(-c3ccc(N(c4ccc(-c5ccc6sc7ccccc7c6c5)cc4)c4ccc5ccccc5c4)cc3)ccc3ccccc23)cc1 ; c1ccc(-c2cccc3ccc(-c4ccc(N(c5ccc(-c6ccc7sc8ccccc8c7c6)cc5)c5cccc6ccccc56)cc4)cc23)cc1 ; c1ccc(-c2cccc3ccc(-c4ccc(N(c5ccc(-c6ccc7sc8ccccc8c7c6)cc5)c5ccc6cccc(-c7ccccc7)c6c5)cc4)cc23)cc1] 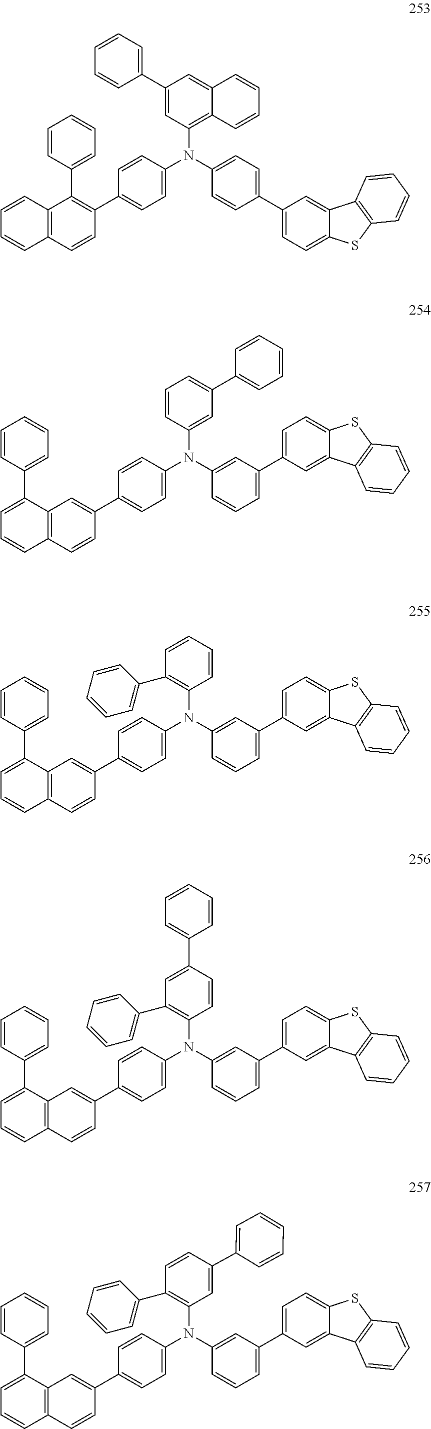 [SMILES: c1ccc(-c2cc(N(c3ccc(-c4ccc5sc6ccccc6c5c4)cc3)c3ccc(-c4ccc5ccccc5c4-c4ccccc4)cc3)c3ccccc3c2)cc1 ; c1ccc(-c2cccc(N(c3ccc(-c4ccc5cccc(-c6ccccc6)c5c4)cc3)c3cccc(-c4ccc5sc6ccccc6c5c4)c3)c2)cc1 ; c1ccc(-c2ccccc2N(c2ccc(-c3ccc4cccc(-c5ccccc5)c4c3)cc2)c2cccc(-c3ccc4sc5ccccc5c4c3)c2)cc1 ; c1ccc(-c2ccc(N(c3ccc(-c4ccc5cccc(-c6ccccc6)c5c4)cc3)c3cccc(-c4ccc5sc6ccccc6c5c4)c3)c(-c3ccccc3)c2)cc1 ; c1ccc(-c2ccc(-c3ccccc3)c(N(c3ccc(-c4ccc5cccc(-c6ccccc6)c5c4)cc3)c3cccc(-c4ccc5sc6ccccc6c5c4)c3)c2)cc1] 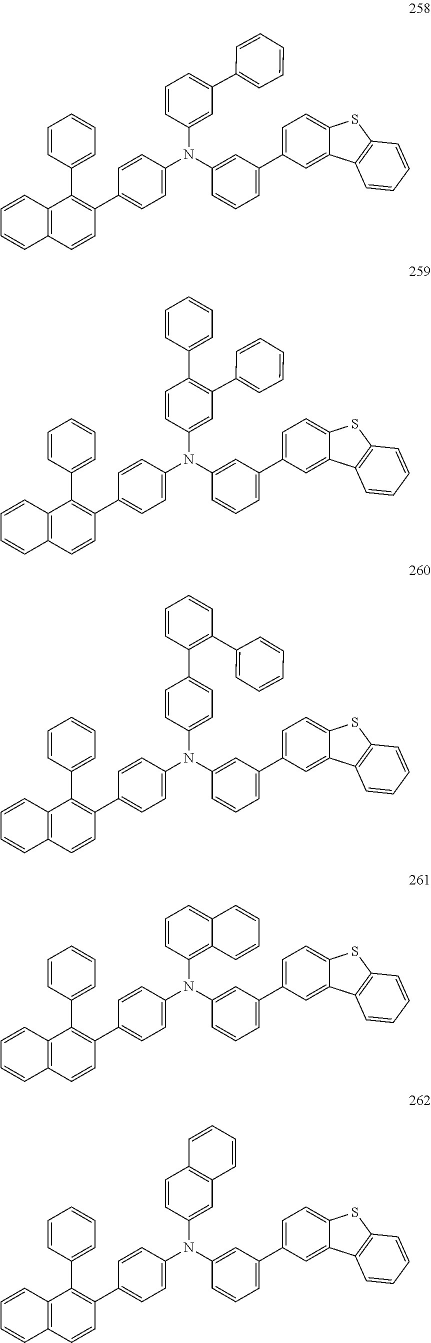 [SMILES: c1ccc(-c2cccc(N(c3ccc(-c4ccc5ccccc5c4-c4ccccc4)cc3)c3cccc(-c4ccc5sc6ccccc6c5c4)c3)c2)cc1 ; c1ccc(-c2ccc(N(c3ccc(-c4ccc5ccccc5c4-c4ccccc4)cc3)c3cccc(-c4ccc5sc6ccccc6c5c4)c3)cc2-c2ccccc2)cc1 ; c1ccc(-c2ccccc2-c2ccc(N(c3ccc(-c4ccc5ccccc5c4-c4ccccc4)cc3)c3cccc(-c4ccc5sc6ccccc6c5c4)c3)cc2)cc1 ; c1ccc(-c2c(-c3ccc(N(c4cccc(-c5ccc6sc7ccccc7c6c5)c4)c4cccc5ccccc45)cc3)ccc3ccccc23)cc1 ; c1ccc(-c2c(-c3ccc(N(c4cccc(-c5ccc6sc7ccccc7c6c5)c4)c4ccc5ccccc5c4)cc3)ccc3ccccc23)cc1] 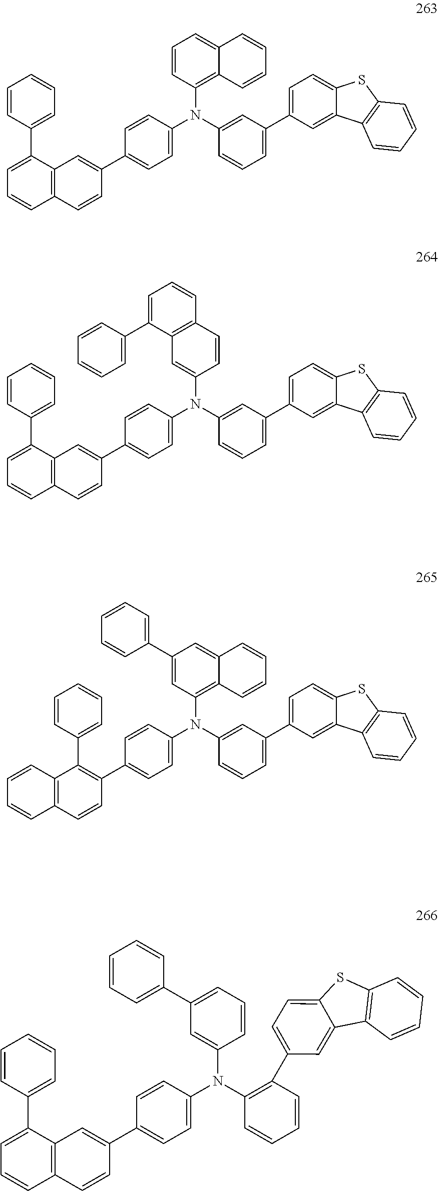 [SMILES: c1ccc(-c2cccc3ccc(-c4ccc(N(c5cccc(-c6ccc7sc8ccccc8c7c6)c5)c5cccc6ccccc56)cc4)cc23)cc1 ; c1ccc(-c2cccc3ccc(-c4ccc(N(c5cccc(-c6ccc7sc8ccccc8c7c6)c5)c5ccc6cccc(-c7ccccc7)c6c5)cc4)cc23)cc1 ; c1ccc(-c2cc(N(c3ccc(-c4ccc5ccccc5c4-c4ccccc4)cc3)c3cccc(-c4ccc5sc6ccccc6c5c4)c3)c3ccccc3c2)cc1 ; c1ccc(-c2cccc(N(c3ccc(-c4ccc5cccc(-c6ccccc6)c5c4)cc3)c3ccccc3-c3ccc4sc5ccccc5c4c3)c2)cc1] 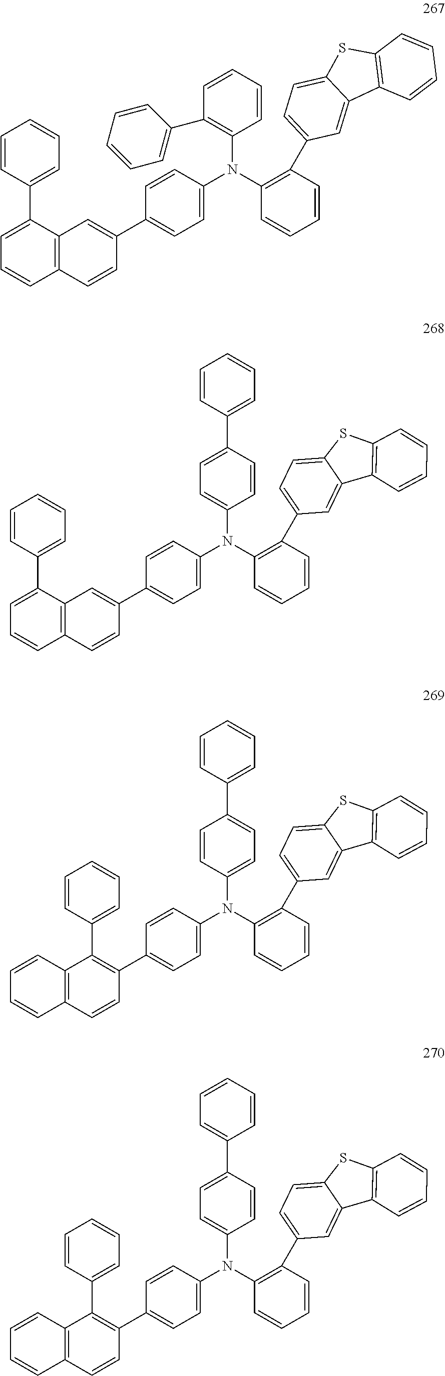 [SMILES: c1ccc(-c2ccccc2N(c2ccc(-c3ccc4cccc(-c5ccccc5)c4c3)cc2)c2ccccc2-c2ccc3sc4ccccc4c3c2)cc1 ; c1ccc(-c2ccc(N(c3ccc(-c4ccc5cccc(-c6ccccc6)c5c4)cc3)c3ccccc3-c3ccc4sc5ccccc5c4c3)cc2)cc1 ; c1ccc(-c2ccc(N(c3ccc(-c4ccc5ccccc5c4-c4ccccc4)cc3)c3ccccc3-c3ccc4sc5ccccc5c4c3)cc2)cc1 ; c1ccc(-c2ccc(N(c3ccc(-c4ccc5ccccc5c4-c4ccccc4)cc3)c3ccccc3-c3ccc4sc5ccccc5c4c3)cc2)cc1] 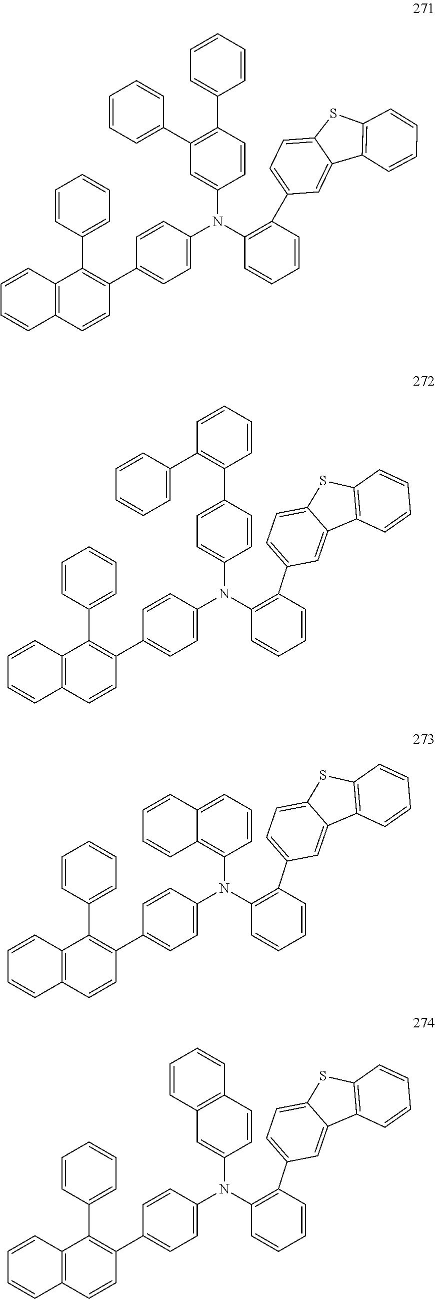 [SMILES: c1ccc(-c2ccc(N(c3ccc(-c4ccc5ccccc5c4-c4ccccc4)cc3)c3ccccc3-c3ccc4sc5ccccc5c4c3)cc2-c2ccccc2)cc1 ; c1ccc(-c2ccccc2-c2ccc(N(c3ccc(-c4ccc5ccccc5c4-c4ccccc4)cc3)c3ccccc3-c3ccc4sc5ccccc5c4c3)cc2)cc1 ; c1ccc(-c2c(-c3ccc(N(c4ccccc4-c4ccc5sc6ccccc6c5c4)c4cccc5ccccc45)cc3)ccc3ccccc23)cc1 ; c1ccc(-c2c(-c3ccc(N(c4ccc5ccccc5c4)c4ccccc4-c4ccc5sc6ccccc6c5c4)cc3)ccc3ccccc23)cc1] 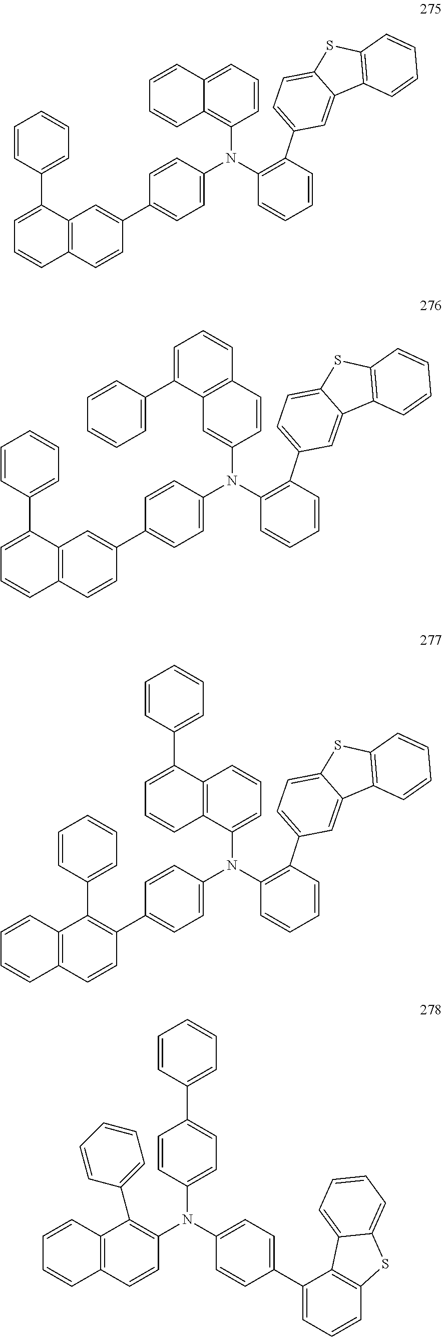 [SMILES: c1ccc(-c2cccc3ccc(-c4ccc(N(c5ccccc5-c5ccc6sc7ccccc7c6c5)c5cccc6ccccc56)cc4)cc23)cc1 ; c1ccc(-c2cccc3ccc(-c4ccc(N(c5ccc6cccc(-c7ccccc7)c6c5)c5ccccc5-c5ccc6sc7ccccc7c6c5)cc4)cc23)cc1 ; c1ccc(-c2c(-c3ccc(N(c4ccccc4-c4ccc5sc6ccccc6c5c4)c4cccc5c(-c6ccccc6)cccc45)cc3)ccc3ccccc23)cc1 ; c1ccc(-c2ccc(N(c3ccc(-c4cccc5sc6ccccc6c45)cc3)c3ccc4ccccc4c3-c3ccccc3)cc2)cc1] 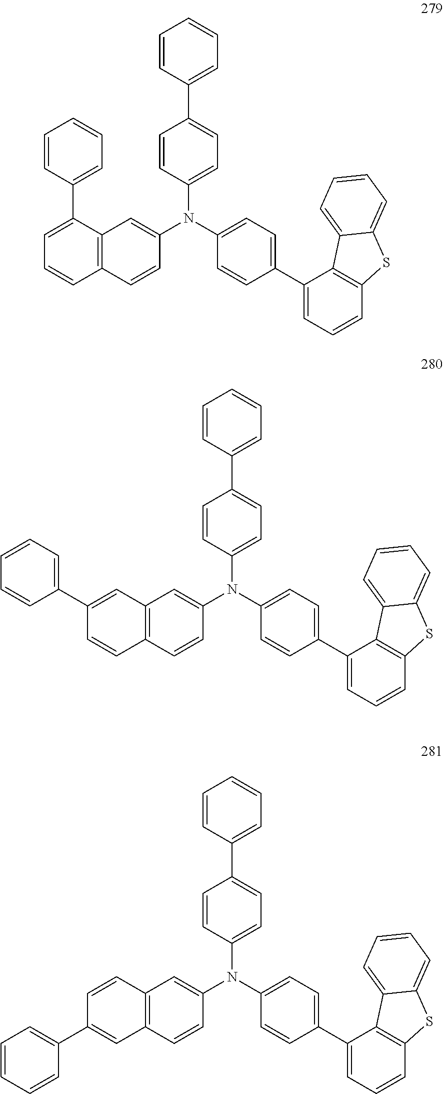 [SMILES: c1ccc(-c2ccc(N(c3ccc(-c4cccc5sc6ccccc6c45)cc3)c3ccc4cccc(-c5ccccc5)c4c3)cc2)cc1 ; c1ccc(-c2ccc(N(c3ccc(-c4cccc5sc6ccccc6c45)cc3)c3ccc4ccc(-c5ccccc5)cc4c3)cc2)cc1 ; c1ccc(-c2ccc(N(c3ccc(-c4cccc5sc6ccccc6c45)cc3)c3ccc4cc(-c5ccccc5)ccc4c3)cc2)cc1] 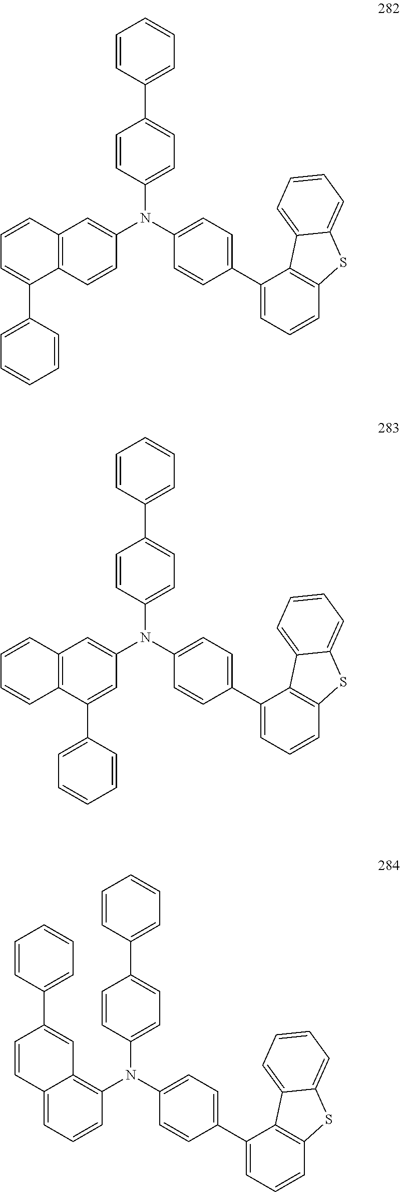 [SMILES: c1ccc(-c2ccc(N(c3ccc(-c4cccc5sc6ccccc6c45)cc3)c3ccc4c(-c5ccccc5)cccc4c3)cc2)cc1 ; c1ccc(-c2ccc(N(c3ccc(-c4cccc5sc6ccccc6c45)cc3)c3cc(-c4ccccc4)c4ccccc4c3)cc2)cc1 ; c1ccc(-c2ccc(N(c3ccc(-c4cccc5sc6ccccc6c45)cc3)c3cccc4ccc(-c5ccccc5)cc34)cc2)cc1] 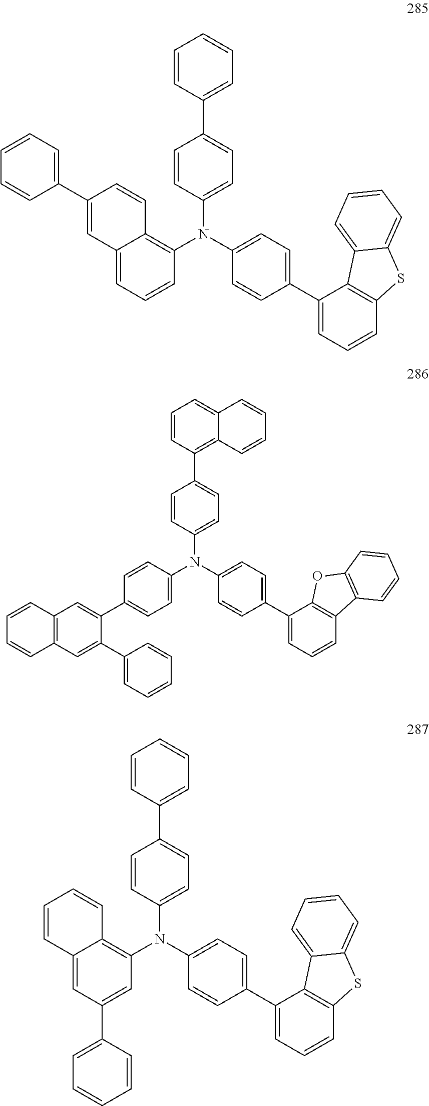 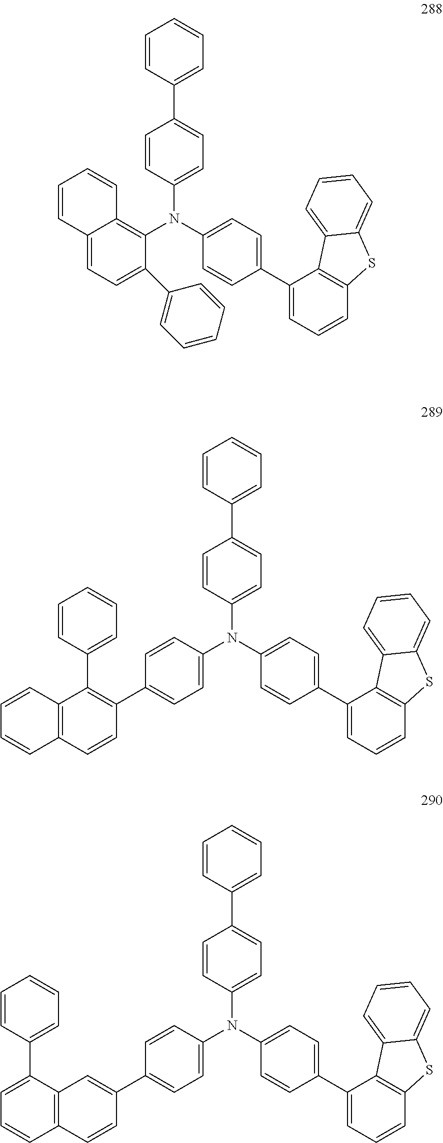 [SMILES: c1ccc(-c2ccc(N(c3ccc(-c4cccc5sc6ccccc6c45)cc3)c3c(-c4ccccc4)ccc4ccccc34)cc2)cc1 ; c1ccc(-c2ccc(N(c3ccc(-c4ccc5ccccc5c4-c4ccccc4)cc3)c3ccc(-c4cccc5sc6ccccc6c45)cc3)cc2)cc1 ; c1ccc(-c2ccc(N(c3ccc(-c4ccc5cccc(-c6ccccc6)c5c4)cc3)c3ccc(-c4cccc5sc6ccccc6c45)cc3)cc2)cc1] 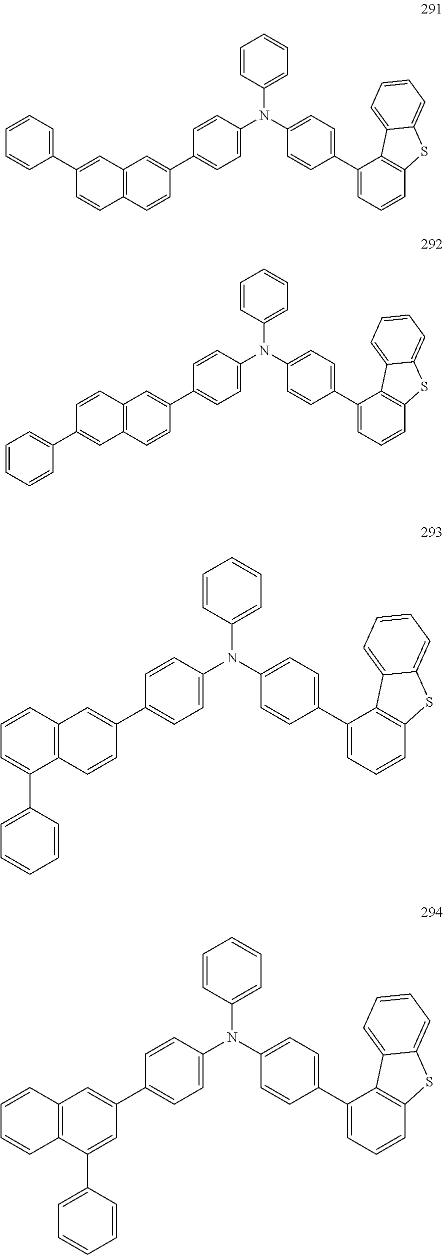 [SMILES: c1ccc(-c2ccc3ccc(-c4ccc(N(c5ccccc5)c5ccc(-c6cccc7sc8ccccc8c67)cc5)cc4)cc3c2)cc1 ; c1ccc(-c2ccc3cc(-c4ccc(N(c5ccccc5)c5ccc(-c6cccc7sc8ccccc8c67)cc5)cc4)ccc3c2)cc1 ; c1ccc(-c2cccc3cc(-c4ccc(N(c5ccccc5)c5ccc(-c6cccc7sc8ccccc8c67)cc5)cc4)ccc23)cc1 ; c1ccc(-c2cc(-c3ccc(N(c4ccccc4)c4ccc(-c5cccc6sc7ccccc7c56)cc4)cc3)cc3ccccc23)cc1] 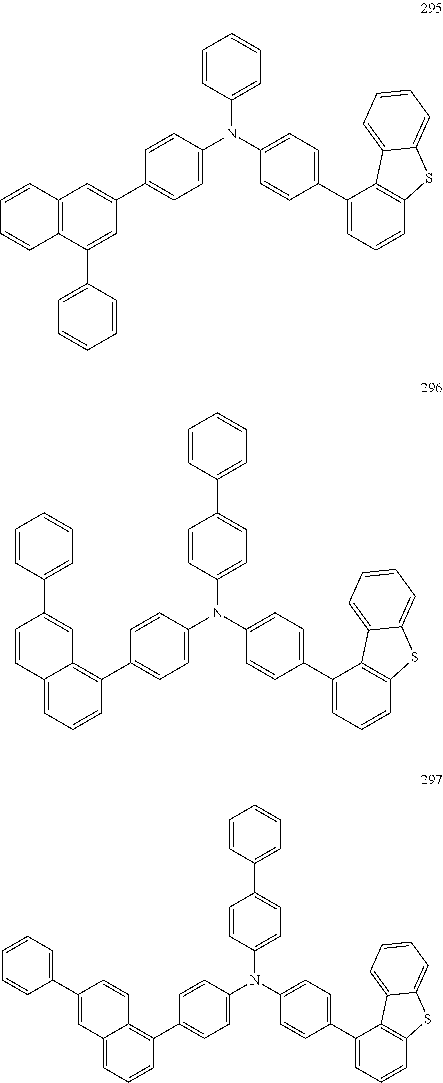 [SMILES: c1ccc(-c2cc(-c3ccc(N(c4ccccc4)c4ccc(-c5cccc6sc7ccccc7c56)cc4)cc3)cc3ccccc23)cc1 ; c1ccc(-c2ccc(N(c3ccc(-c4cccc5ccc(-c6ccccc6)cc45)cc3)c3ccc(-c4cccc5sc6ccccc6c45)cc3)cc2)cc1 ; c1ccc(-c2ccc(N(c3ccc(-c4cccc5cc(-c6ccccc6)ccc45)cc3)c3ccc(-c4cccc5sc6ccccc6c45)cc3)cc2)cc1] 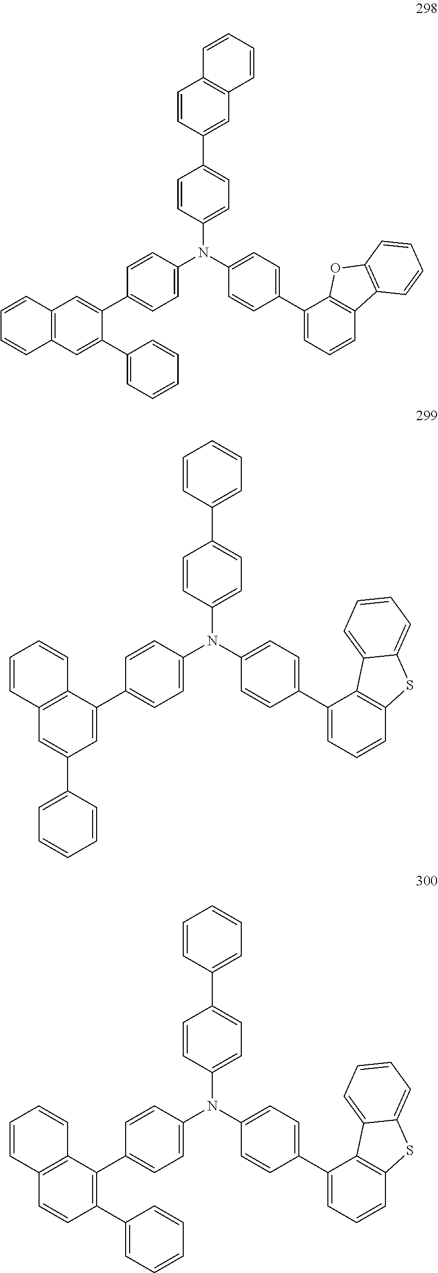 [SMILES: c1ccc(-c2cc3ccccc3cc2-c2ccc(N(c3ccc(-c4ccc5ccccc5c4)cc3)c3ccc(-c4cccc5c4oc4ccccc45)cc3)cc2)cc1 ; c1ccc(-c2ccc(N(c3ccc(-c4cc(-c5ccccc5)cc5ccccc45)cc3)c3ccc(-c4cccc5sc6ccccc6c45)cc3)cc2)cc1 ; c1ccc(-c2ccc(N(c3ccc(-c4c(-c5ccccc5)ccc5ccccc45)cc3)c3ccc(-c4cccc5sc6ccccc6c45)cc3)cc2)cc1] 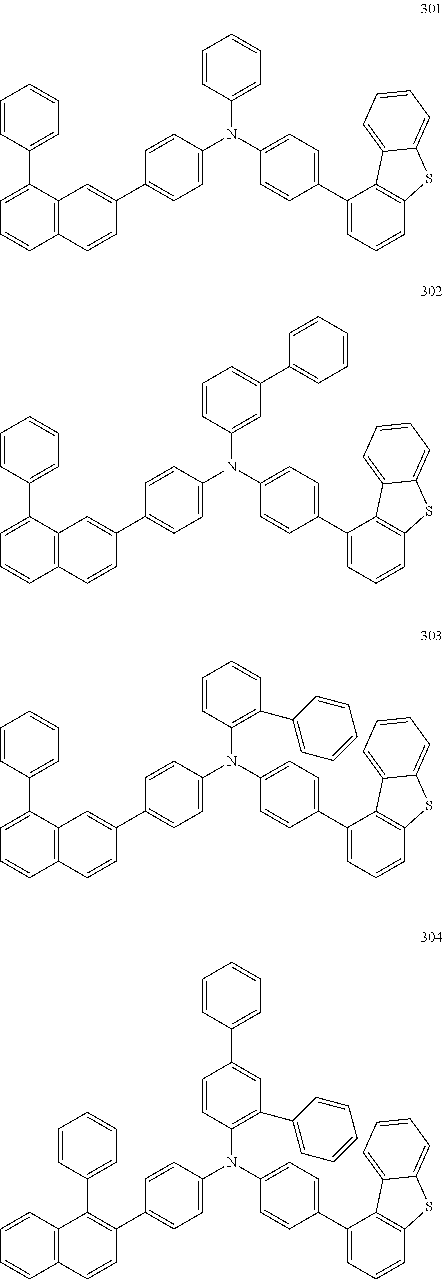 [SMILES: c1ccc(-c2cccc3ccc(-c4ccc(N(c5ccccc5)c5ccc(-c6cccc7sc8ccccc8c67)cc5)cc4)cc23)cc1 ; c1ccc(-c2cccc(N(c3ccc(-c4ccc5cccc(-c6ccccc6)c5c4)cc3)c3ccc(-c4cccc5sc6ccccc6c45)cc3)c2)cc1 ; c1ccc(-c2ccccc2N(c2ccc(-c3ccc4cccc(-c5ccccc5)c4c3)cc2)c2ccc(-c3cccc4sc5ccccc5c34)cc2)cc1 ; c1ccc(-c2ccc(N(c3ccc(-c4ccc5ccccc5c4-c4ccccc4)cc3)c3ccc(-c4cccc5sc6ccccc6c45)cc3)c(-c3ccccc3)c2)cc1] 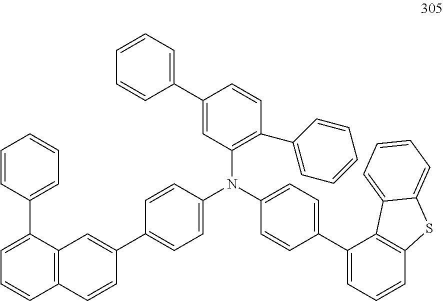 [SMILES: c1ccc(-c2ccc(-c3ccccc3)c(N(c3ccc(-c4ccc5cccc(-c6ccccc6)c5c4)cc3)c3ccc(-c4cccc5sc6ccccc6c45)cc3)c2)cc1] [chemical structure] 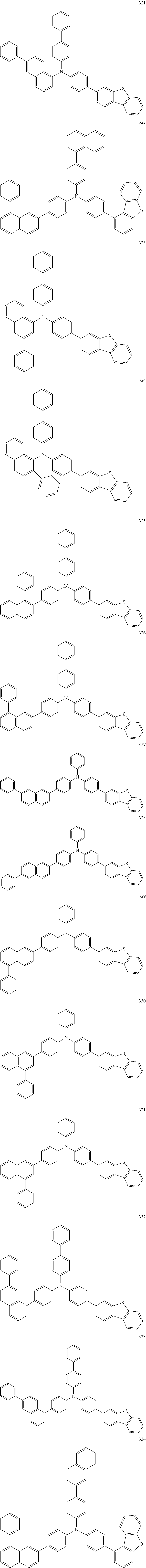 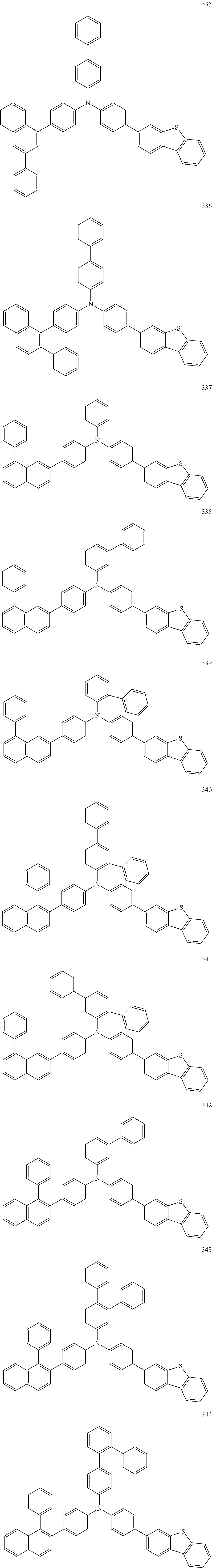 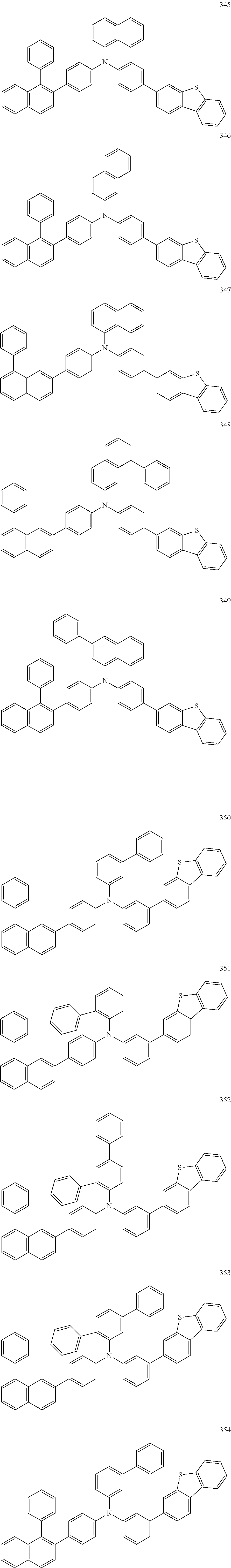 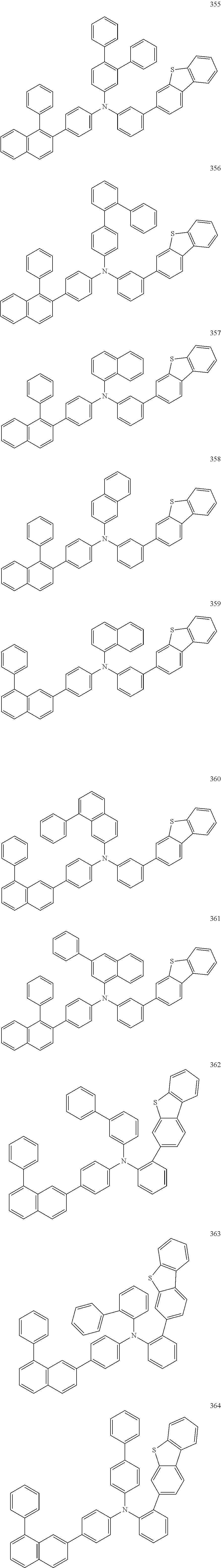 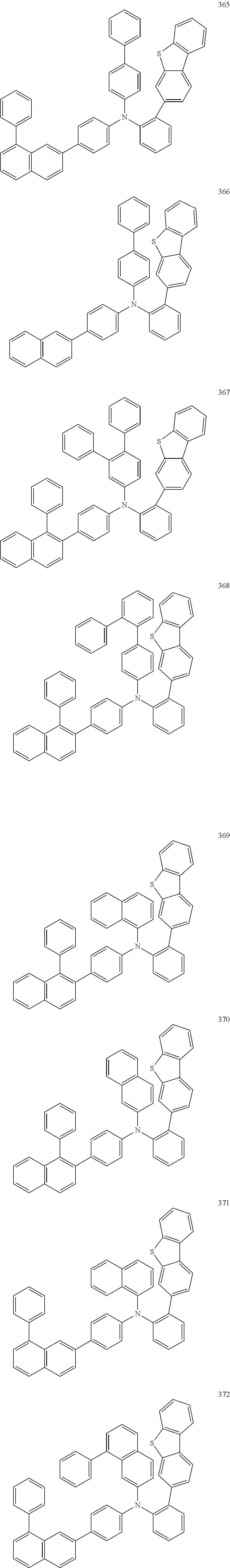 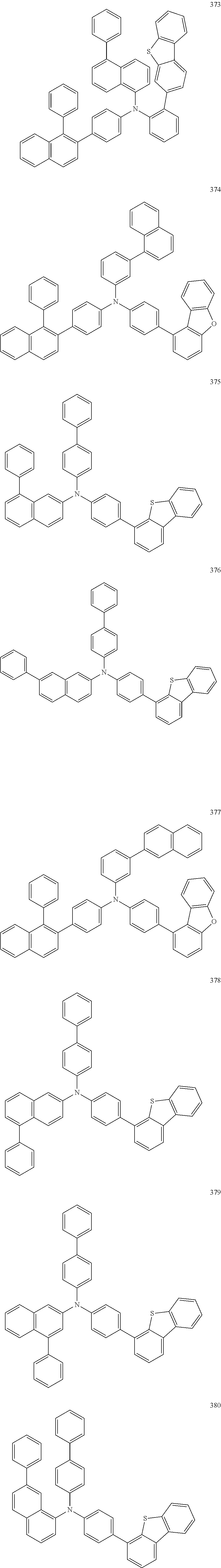 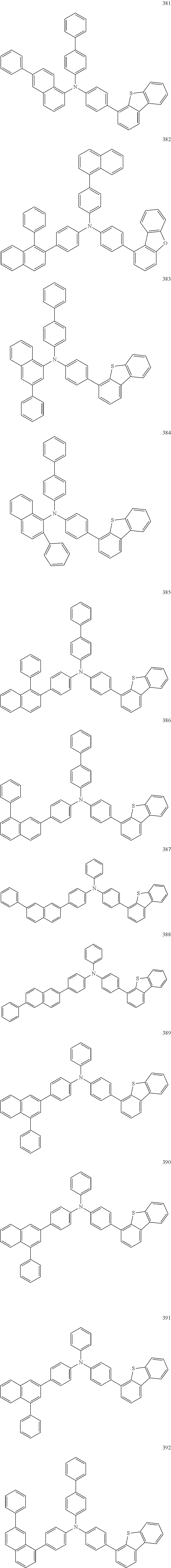 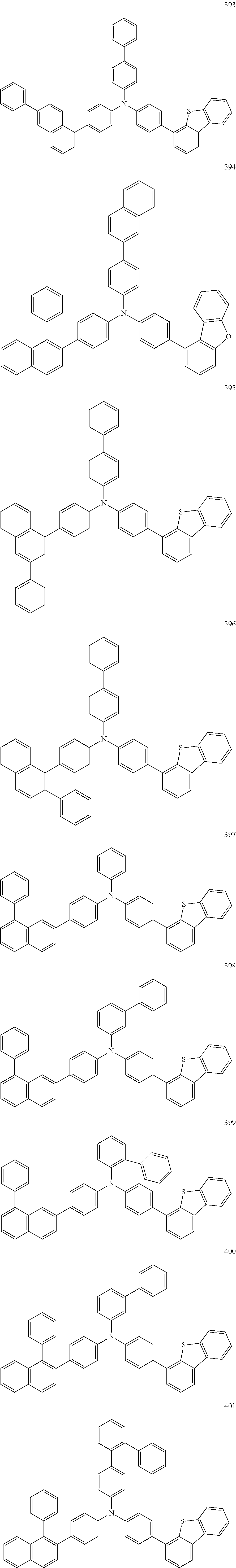 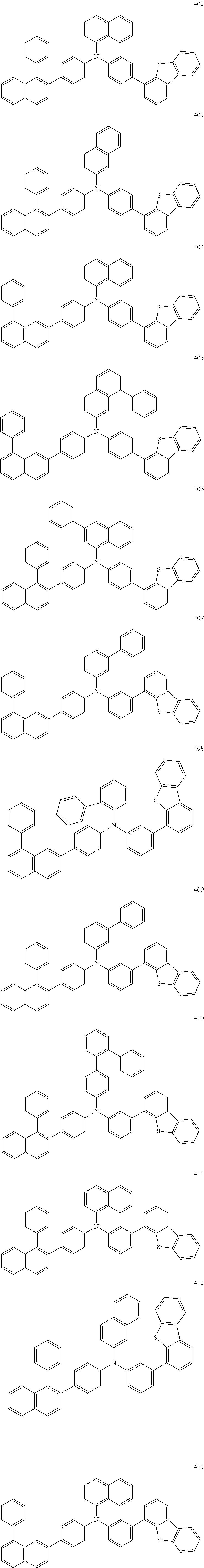 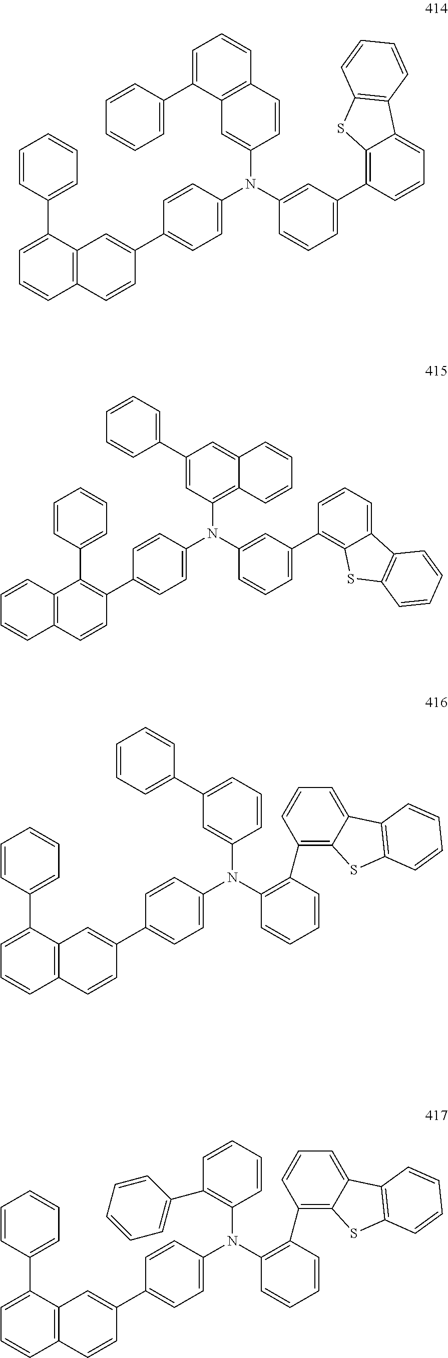 [SMILES: c1ccc(-c2cccc3ccc(-c4ccc(N(c5cccc(-c6cccc7c6sc6ccccc67)c5)c5ccc6cccc(-c7ccccc7)c6c5)cc4)cc23)cc1 ; c1ccc(-c2cc(N(c3ccc(-c4ccc5ccccc5c4-c4ccccc4)cc3)c3cccc(-c4cccc5c4sc4ccccc45)c3)c3ccccc3c2)cc1 ; c1ccc(-c2cccc(N(c3ccc(-c4ccc5cccc(-c6ccccc6)c5c4)cc3)c3ccccc3-c3cccc4c3sc3ccccc34)c2)cc1 ; c1ccc(-c2ccccc2N(c2ccc(-c3ccc4cccc(-c5ccccc5)c4c3)cc2)c2ccccc2-c2cccc3c2sc2ccccc23)cc1] 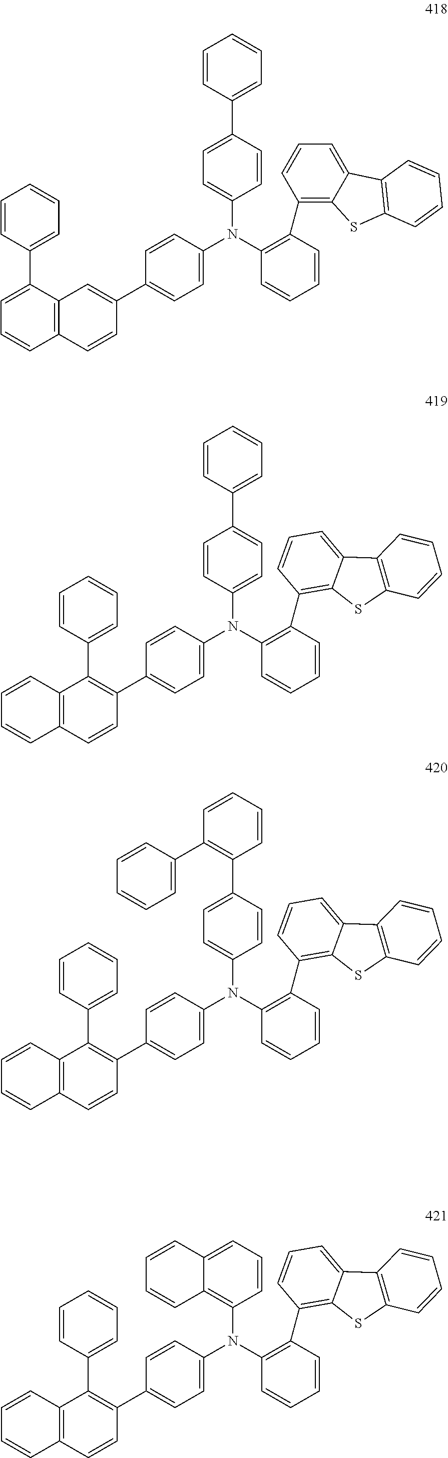 [SMILES: c1ccc(-c2ccc(N(c3ccc(-c4ccc5cccc(-c6ccccc6)c5c4)cc3)c3ccccc3-c3cccc4c3sc3ccccc34)cc2)cc1 ; c1ccc(-c2ccc(N(c3ccc(-c4ccc5ccccc5c4-c4ccccc4)cc3)c3ccccc3-c3cccc4c3sc3ccccc34)cc2)cc1 ; c1ccc(-c2ccccc2-c2ccc(N(c3ccc(-c4ccc5ccccc5c4-c4ccccc4)cc3)c3ccccc3-c3cccc4c3sc3ccccc34)cc2)cc1 ; c1ccc(-c2c(-c3ccc(N(c4ccccc4-c4cccc5c4sc4ccccc45)c4cccc5ccccc45)cc3)ccc3ccccc23)cc1] 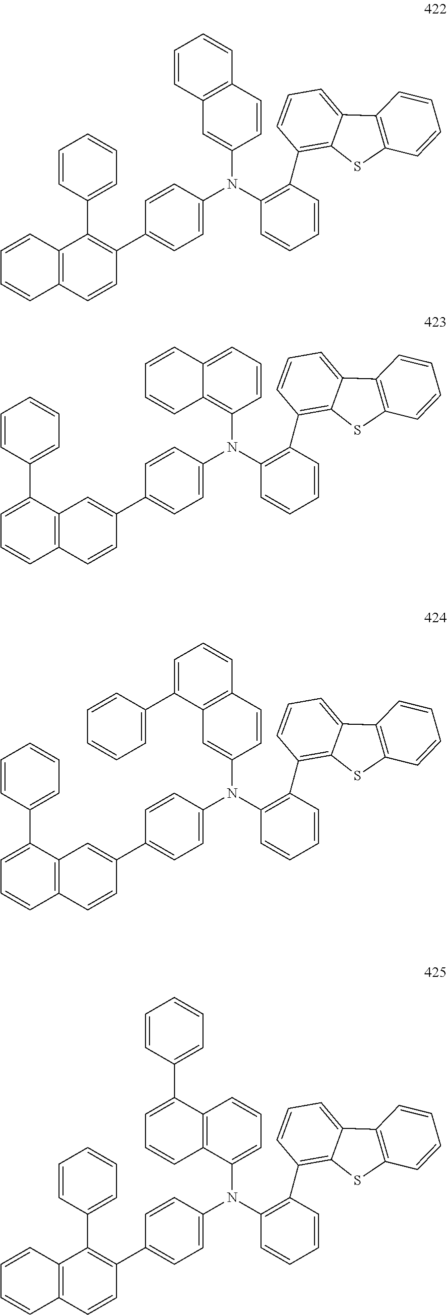 [SMILES: c1ccc(-c2c(-c3ccc(N(c4ccc5ccccc5c4)c4ccccc4-c4cccc5c4sc4ccccc45)cc3)ccc3ccccc23)cc1 ; c1ccc(-c2cccc3ccc(-c4ccc(N(c5ccccc5-c5cccc6c5sc5ccccc56)c5cccc6ccccc56)cc4)cc23)cc1 ; c1ccc(-c2cccc3ccc(-c4ccc(N(c5ccc6cccc(-c7ccccc7)c6c5)c5ccccc5-c5cccc6c5sc5ccccc56)cc4)cc23)cc1 ; c1ccc(-c2c(-c3ccc(N(c4ccccc4-c4cccc5c4sc4ccccc45)c4cccc5c(-c6ccccc6)cccc45)cc3)ccc3ccccc23)cc1]
wherein in Compound Group 1, D is a deuterium atom.

11. An amine compound represented by Formula 1: 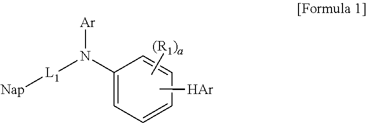
wherein in Formula 1,
L₁ is a direct linkage, a substituted or unsubstituted arylene group having 6 to 30 ring-forming carbon atoms excluding a substituted or unsubstituted naphthyl group, or a substituted or unsubstituted heteroarylene group having 5 to 30 ring-forming carbon atoms,
Ar is a substituted or unsubstituted phenyl group without including an aryl group or a heteroaryl group having 16 or more ring-forming carbon atoms as a substituent, or a substituted or unsubstituted naphthyl group,
a is an integer of 0 to 4,
R₁ is a hydrogen atom, a deuterium atom, a halogen atom, a substituted or unsubstituted alkyl group having 1 to 15 carbon atoms, or a substituted or unsubstituted phenyl group, and
HAr is represented by Formula 2, and Nap is represented by Formula 3 or Formula 4: 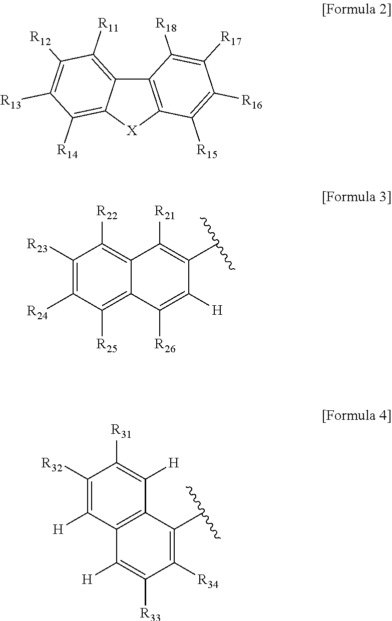
wherein in Formula 2,
X is O or S,
any one of R₁₁ to R₁₄ is a position linked to Formula 1, and the other positions of R₁₁ to R₁₄ excluding the linked position, and R₁₅ to R₁₈ are each independently a hydrogen atom, a deuterium atom, a halogen atom, a substituted or unsubstituted alkyl group having 1 to 15 carbon atoms, or a substituted or unsubstituted phenyl group,
when X is O and when linked to Formula 1 at the R₁₁ position, the R₁₁ position is para to a nitrogen atom of Formula 1,
when linked to Formula 1 at the R₁₂ position, a case in which R₁₅ is a substituted or unsubstituted phenyl group is excluded,
when linked to Formula 1 at the R₁₄ position, Ar of Formula 1 either includes a total of one aryl or heteroaryl group as a substituent or does not include any,
when linked to Formula 1 at the R₁₄ position and when L¹ is a direct linkage, a case in which R₁ is a phenyl group is excluded,
wherein in Formula 3, any one of R₂₁ to R₂₆ is a substituted or unsubstituted phenyl group, and the others are each independently a substituted or unsubstituted alkyl group having 1 to 15 carbon atoms, a halogen atom, a hydrogen atom, or a deuterium atom,
when linked to Formula 1 at the R₁₄ position of Formula 2 and when L¹ is a direct linkage, cases in which R₂₁ or R₂₄ is a substituted or unsubstituted phenyl group are excluded,
wherein in Formula 4, any one of R₃₁ to R₃₄ is a substituted or unsubstituted phenyl group, and the others are each independently a substituted or unsubstituted alkyl group having 1 to 15 carbon atoms, a halogen atom, a hydrogen atom, or a deuterium atom, and
when linked to Formula 1 at the R₁₄ position of Formula 2, a case in which R₃₄ is a substituted or unsubstituted phenyl group is excluded.

12. The amine compound of claim 11, wherein Formula 1 is represented by Formula 1-1 or Formula 1-2: 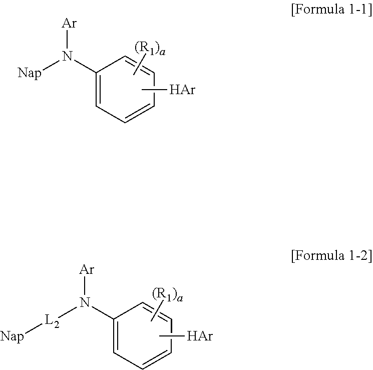
wherein in Formula 1-2, L₂ is a substituted or unsubstituted divalent phenyl, a substituted or unsubstituted divalent biphenyl, or a substituted or unsubstituted divalent dibenzofuran, and
wherein in Formulas 1-1 and 1-2, Nap, Ar, HAr, R₁, and a are the same as defined in Formula 1 above.

13. The amine compound of claim 11, wherein Ar is a substituted or unsubstituted phenyl group, or a substituted or unsubstituted naphthyl group, and
the substituted or unsubstituted substituent is at least one of a hydrogen atom, a deuterium atom, a halogen atom, a phenyl group, a biphenyl group, or a naphthyl group.

14. The amine compound of claim 11, wherein Ar is represented by any one of Ar-a to Ar-q below.

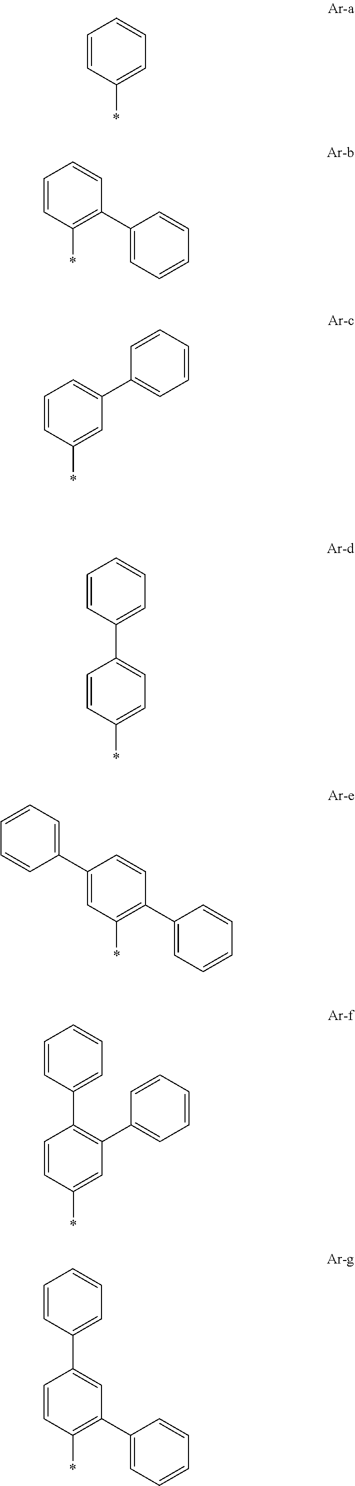

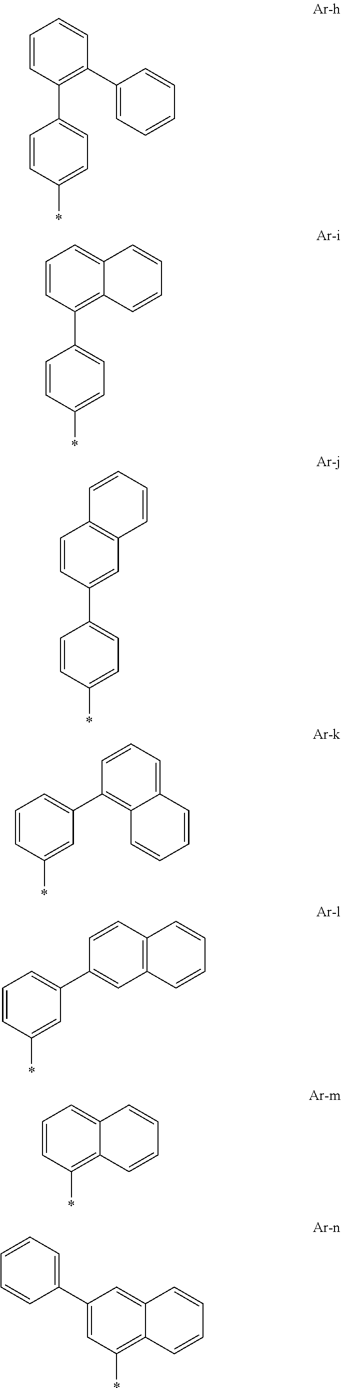

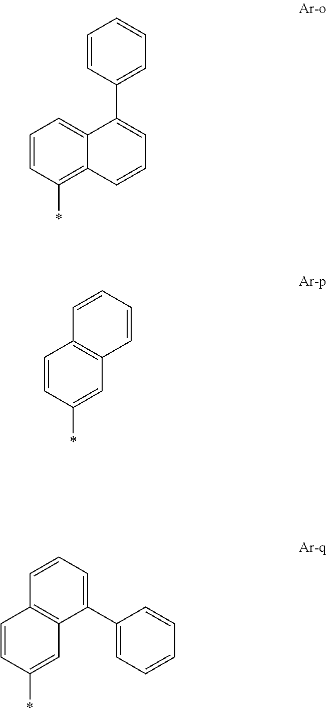 [SMILES: *c1cccc2c(-c3ccccc3)cccc12 ; *c1ccc2ccccc2c1 ; *c1ccc2cccc(-c3ccccc3)c2c1]

15. The amine compound of claim 11, wherein at least one hydrogen atom of the amine compound is substituted with a deuterium atom.

16. The amine compound of claim 11, wherein in Formula 1, Nap, L₁, Ar, HAr, and R₁ do not comprise a substituted or unsubstituted amine group.

17. The amine compound of claim 11, wherein Formula 1 is represented by any one of compounds from Compound Group 1: 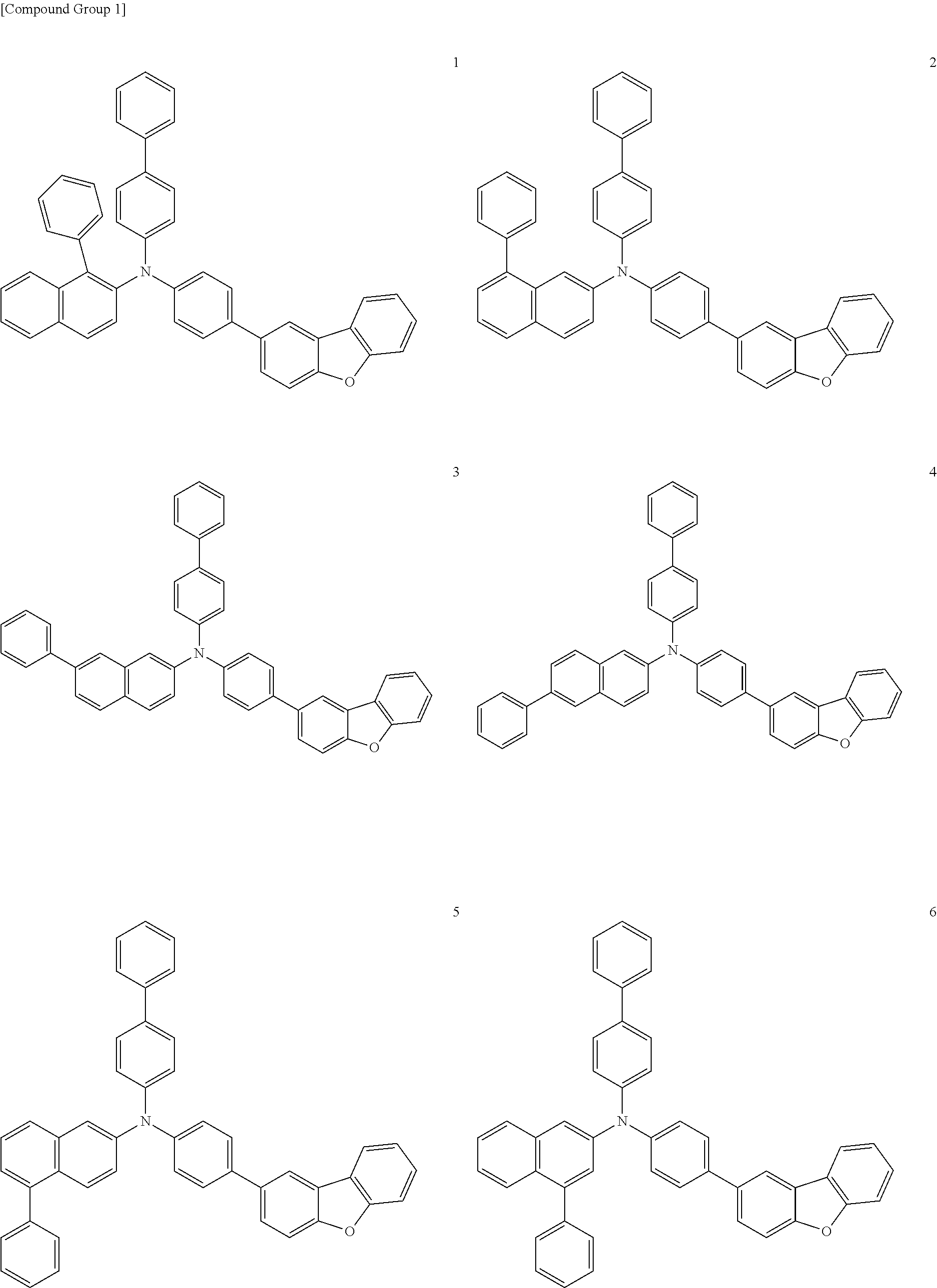 [SMILES: c1ccc(-c2ccc(N(c3ccc(-c4ccc5oc6ccccc6c5c4)cc3)c3ccc4ccccc4c3-c3ccccc3)cc2)cc1 ; c1ccc(-c2ccc(N(c3ccc(-c4ccc5oc6ccccc6c5c4)cc3)c3ccc4cccc(-c5ccccc5)c4c3)cc2)cc1 ; c1ccc(-c2ccc(N(c3ccc(-c4ccc5oc6ccccc6c5c4)cc3)c3ccc4ccc(-c5ccccc5)cc4c3)cc2)cc1 ; c1ccc(-c2ccc(N(c3ccc(-c4ccc5oc6ccccc6c5c4)cc3)c3ccc4cc(-c5ccccc5)ccc4c3)cc2)cc1 ; c1ccc(-c2ccc(N(c3ccc(-c4ccc5oc6ccccc6c5c4)cc3)c3ccc4c(-c5ccccc5)cccc4c3)cc2)cc1 ; c1ccc(-c2ccc(N(c3ccc(-c4ccc5oc6ccccc6c5c4)cc3)c3cc(-c4ccccc4)c4ccccc4c3)cc2)cc1] 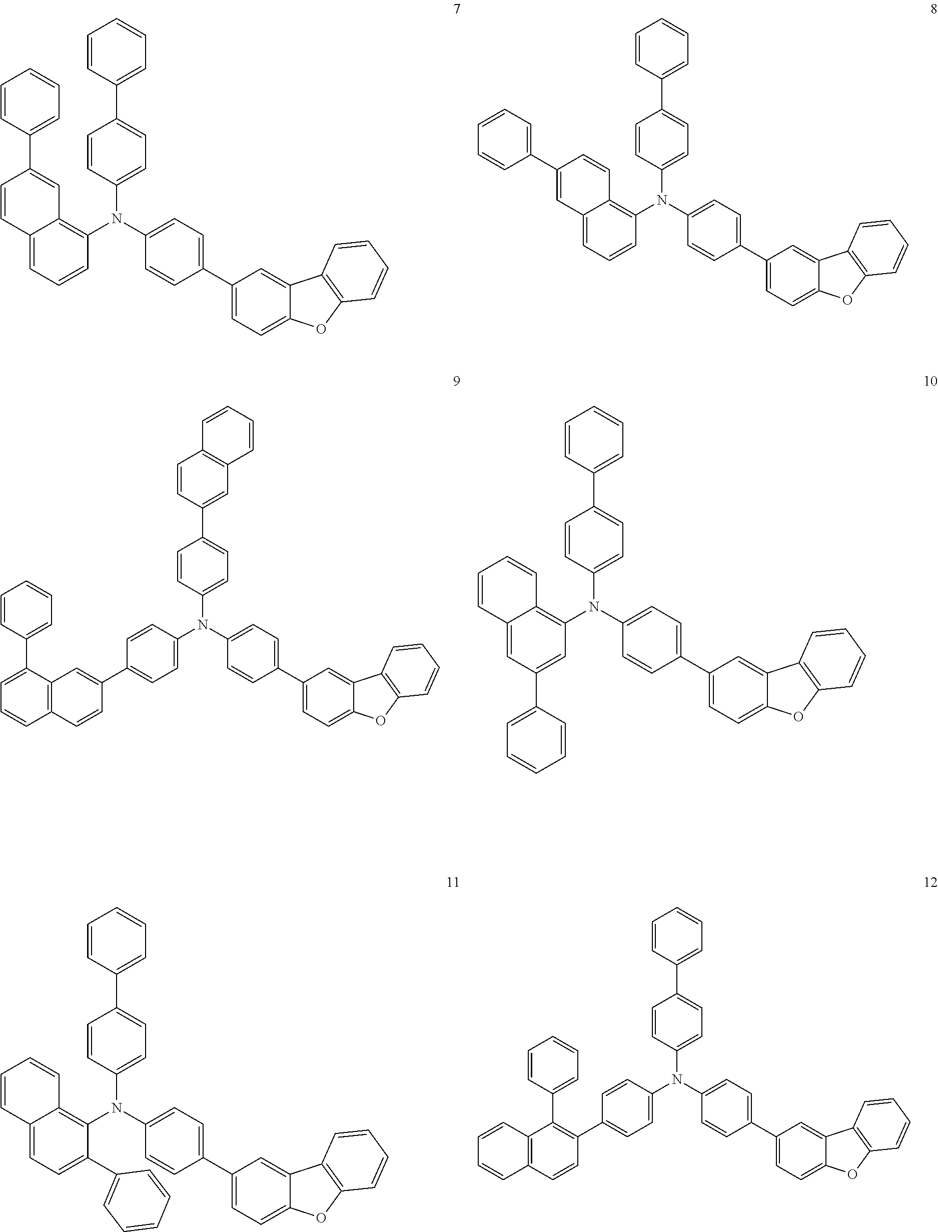 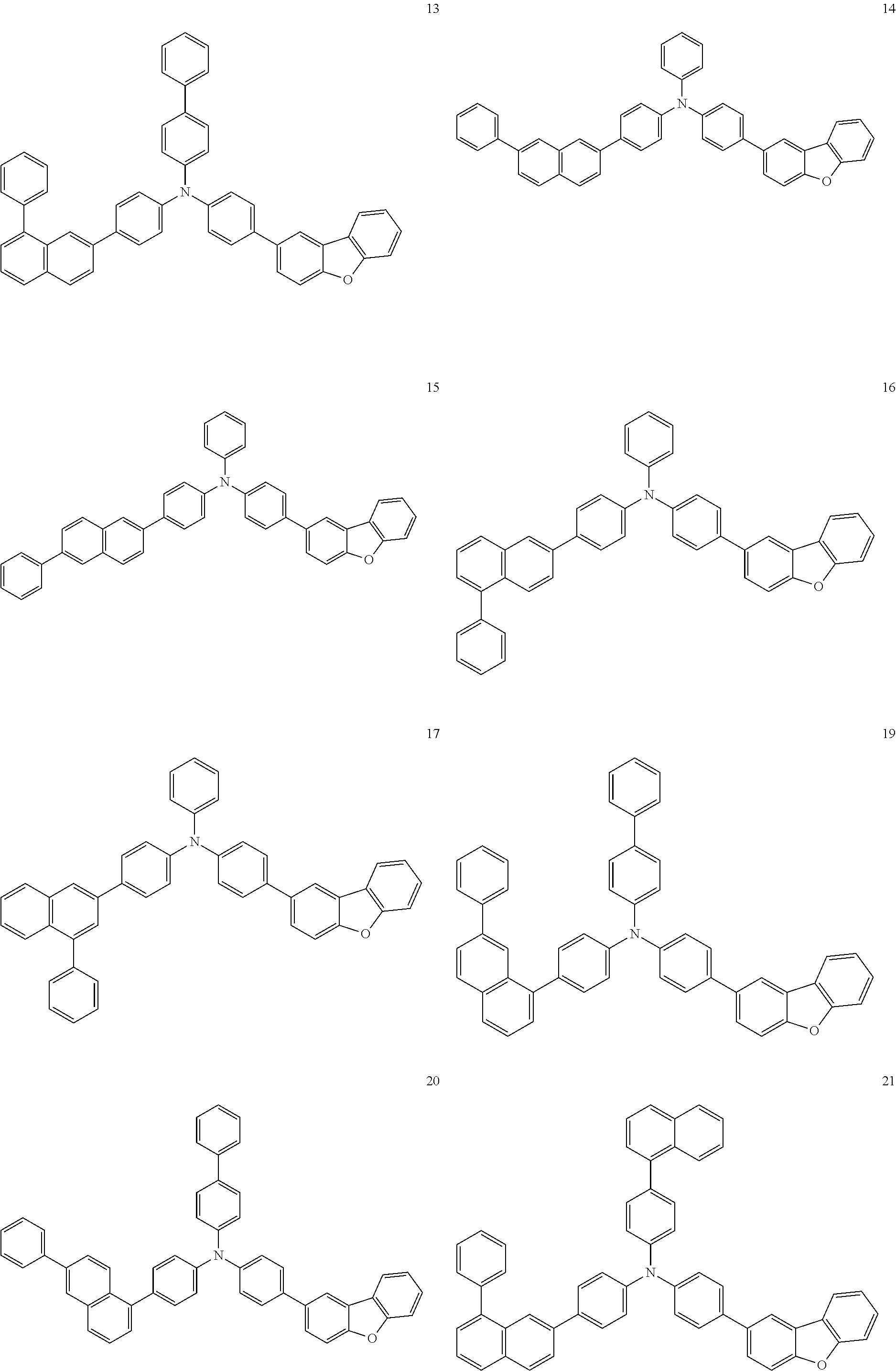 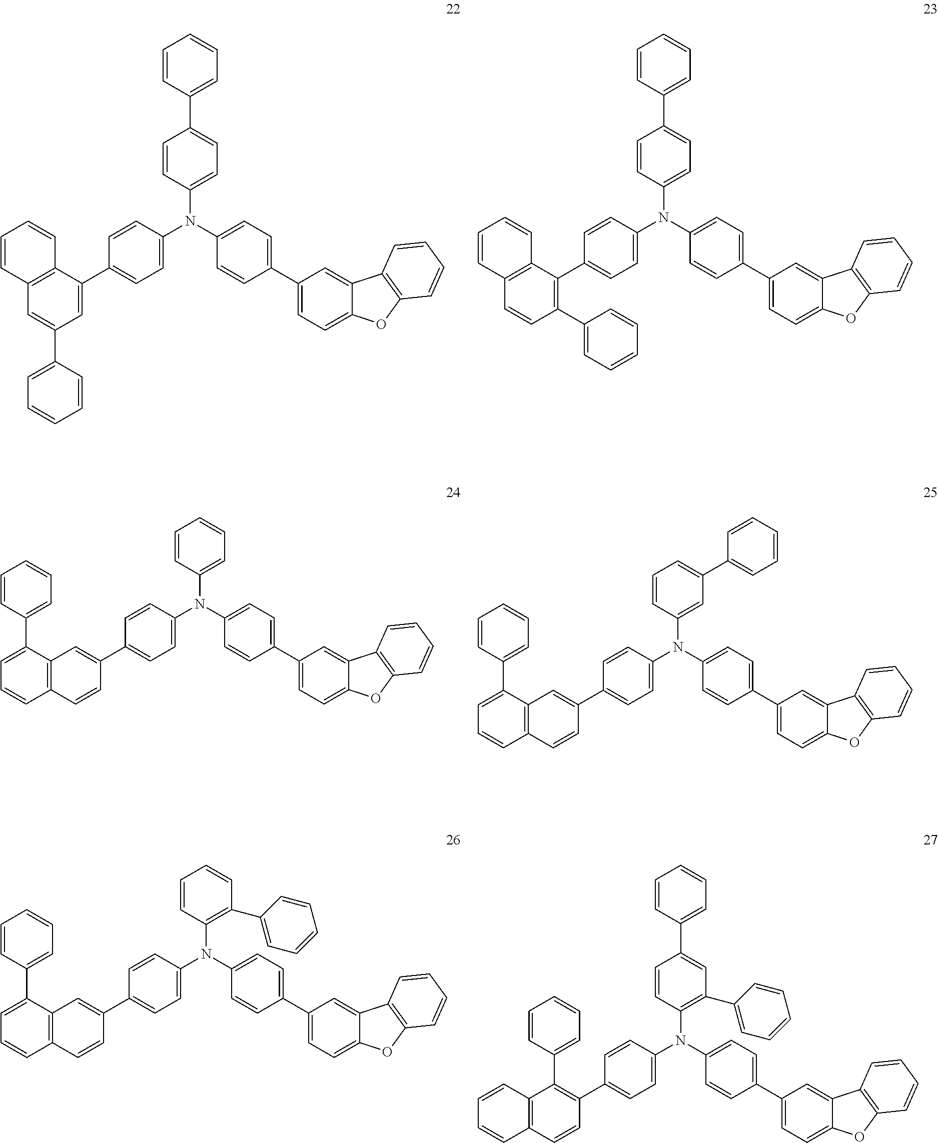 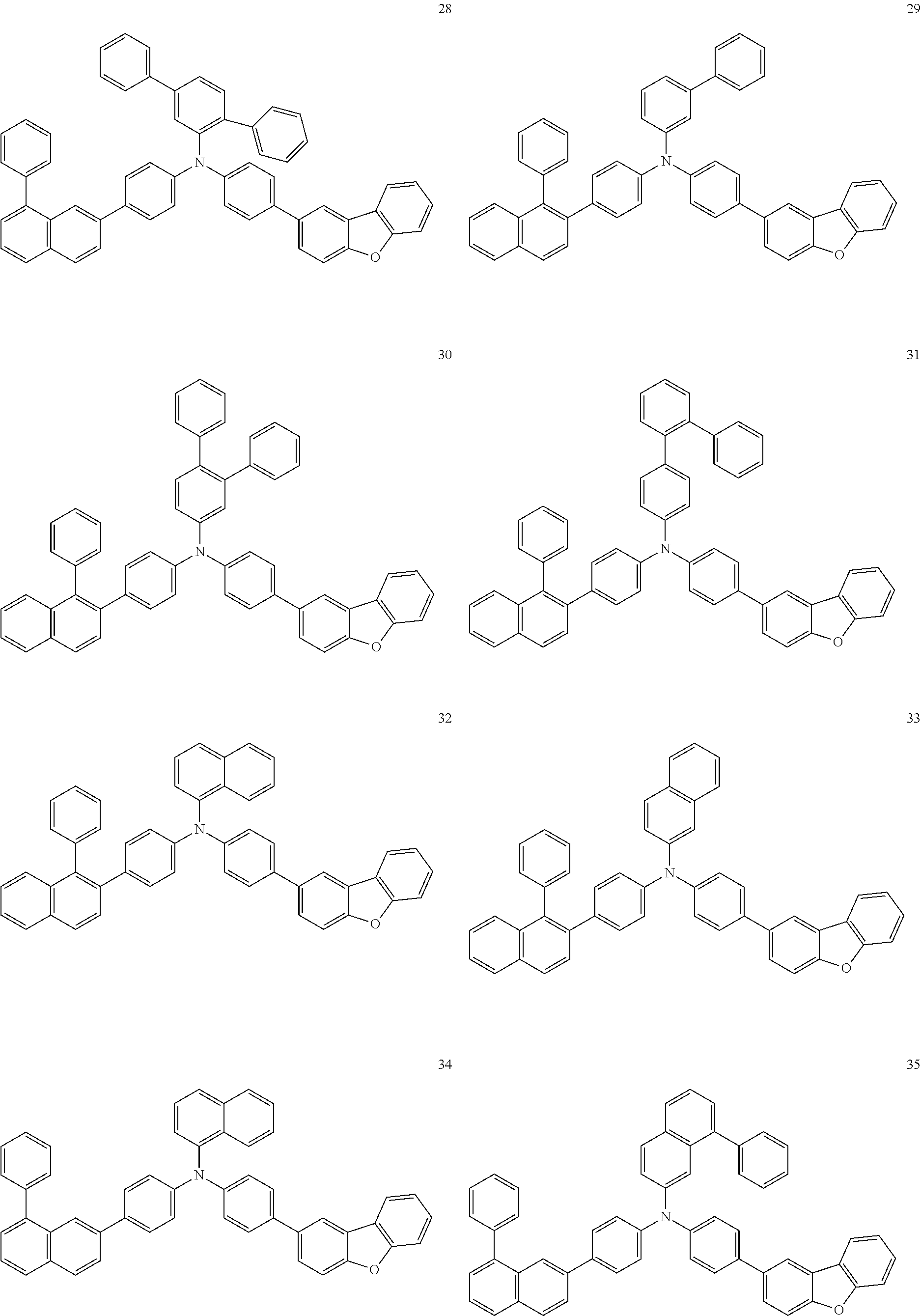 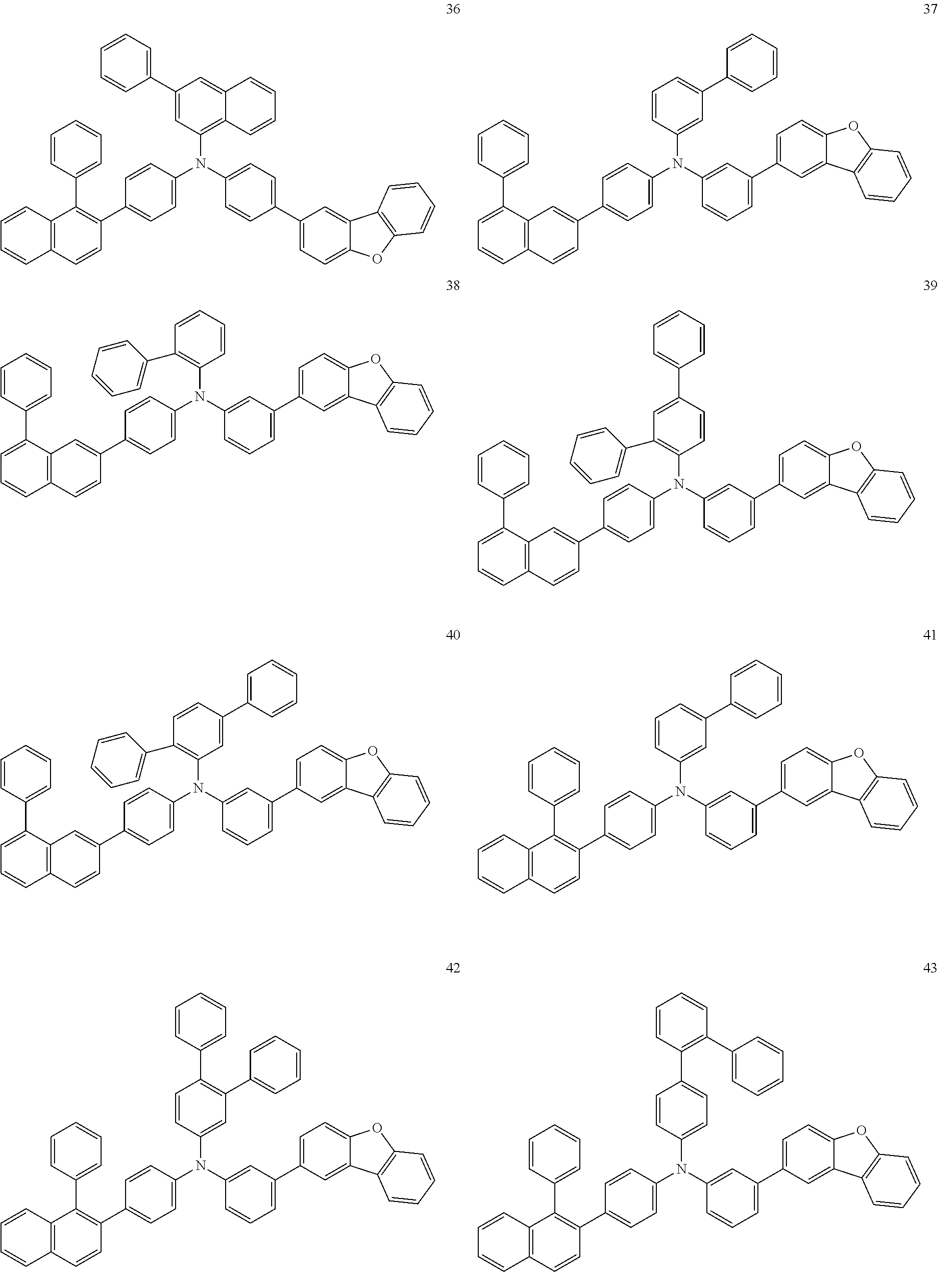 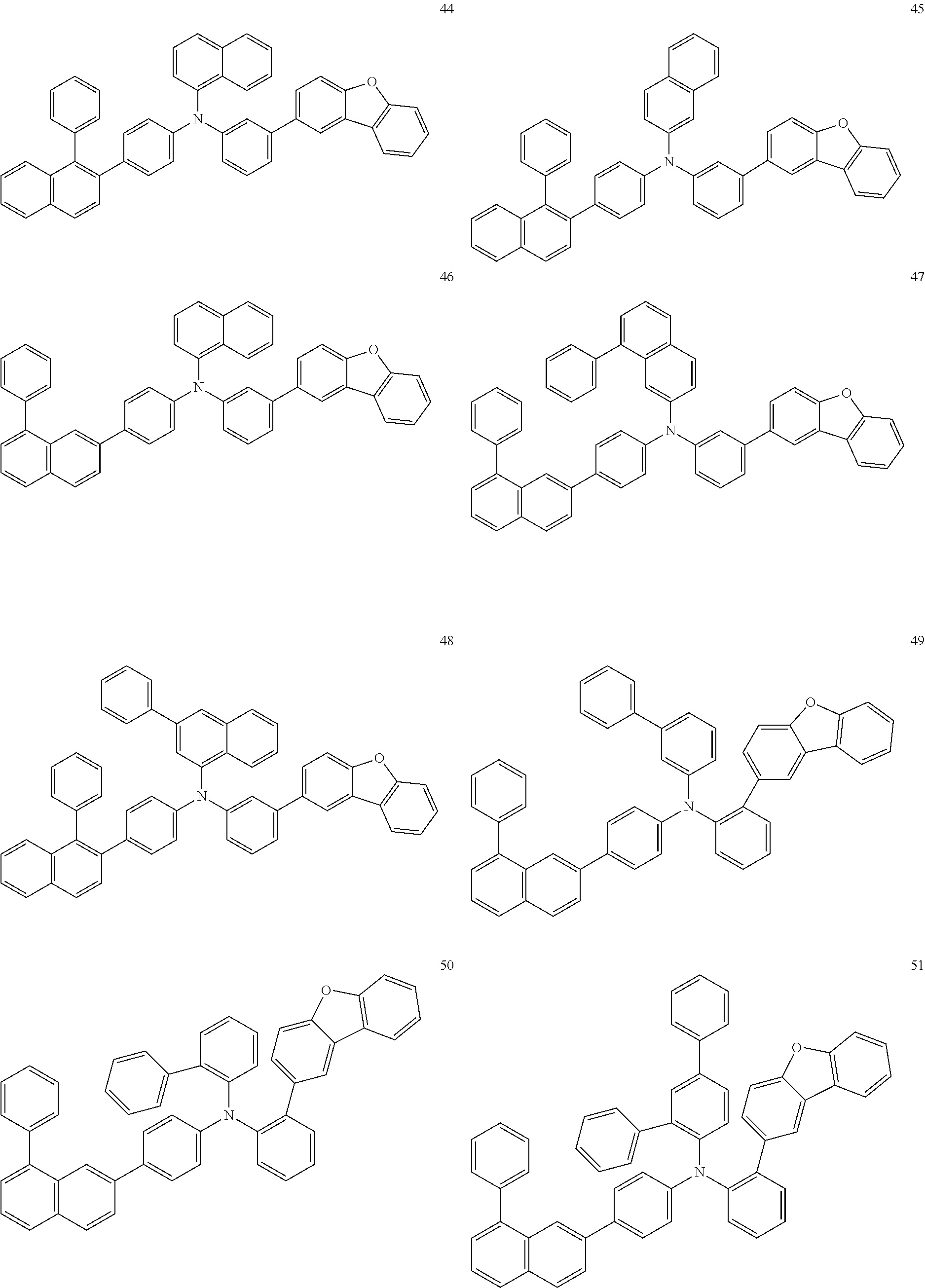 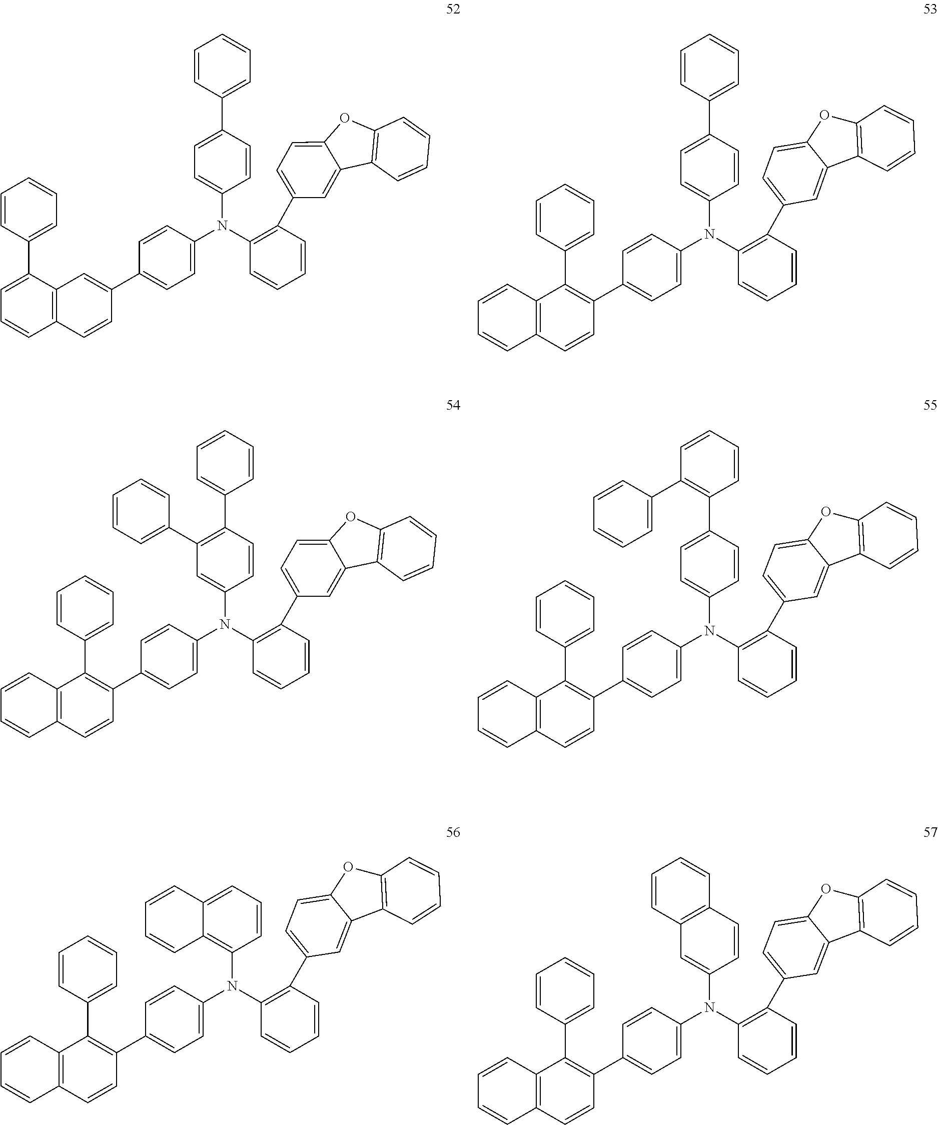 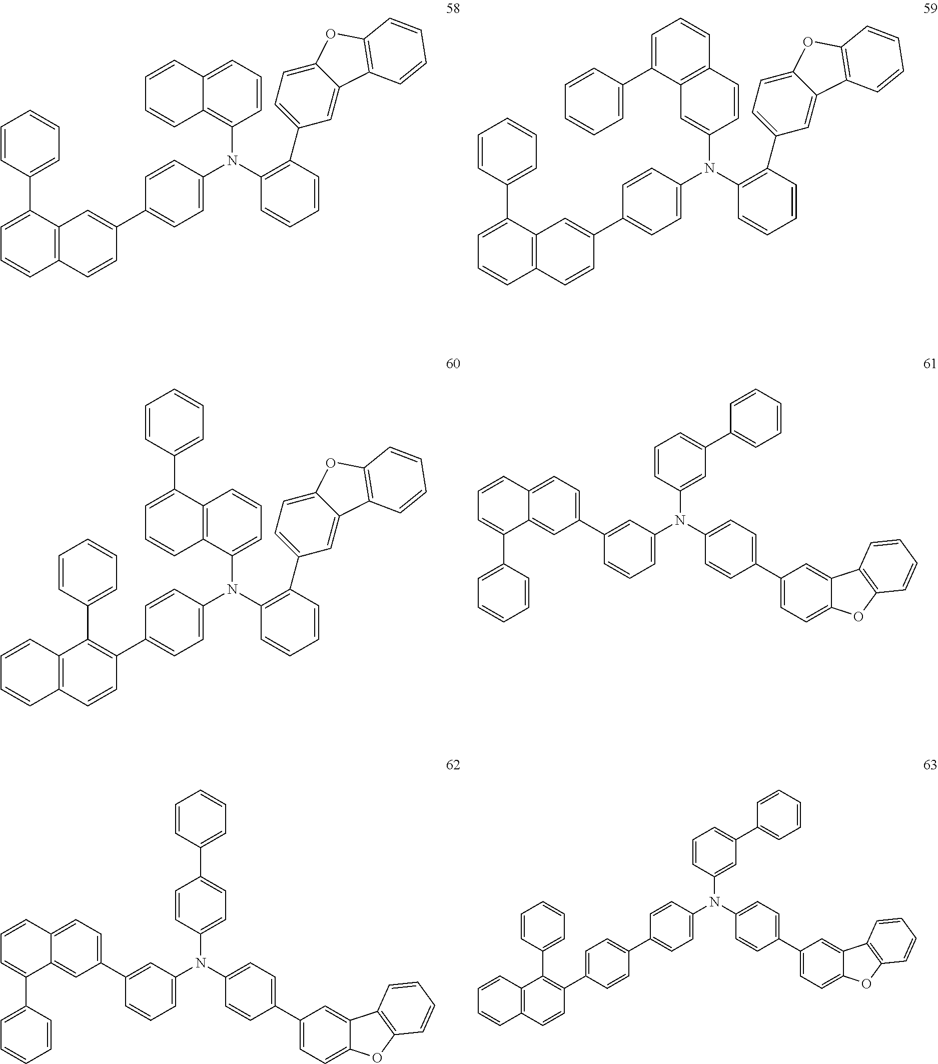 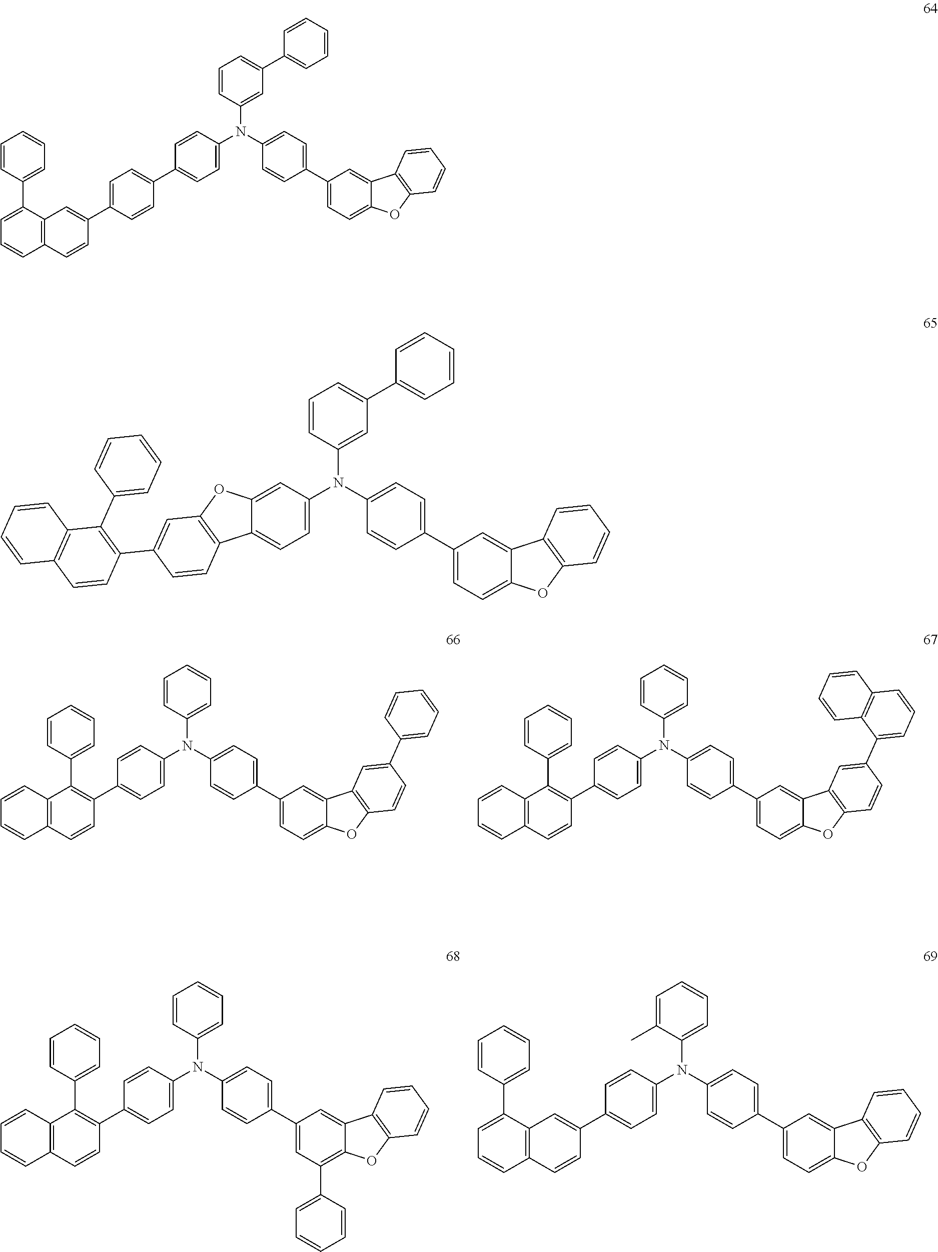 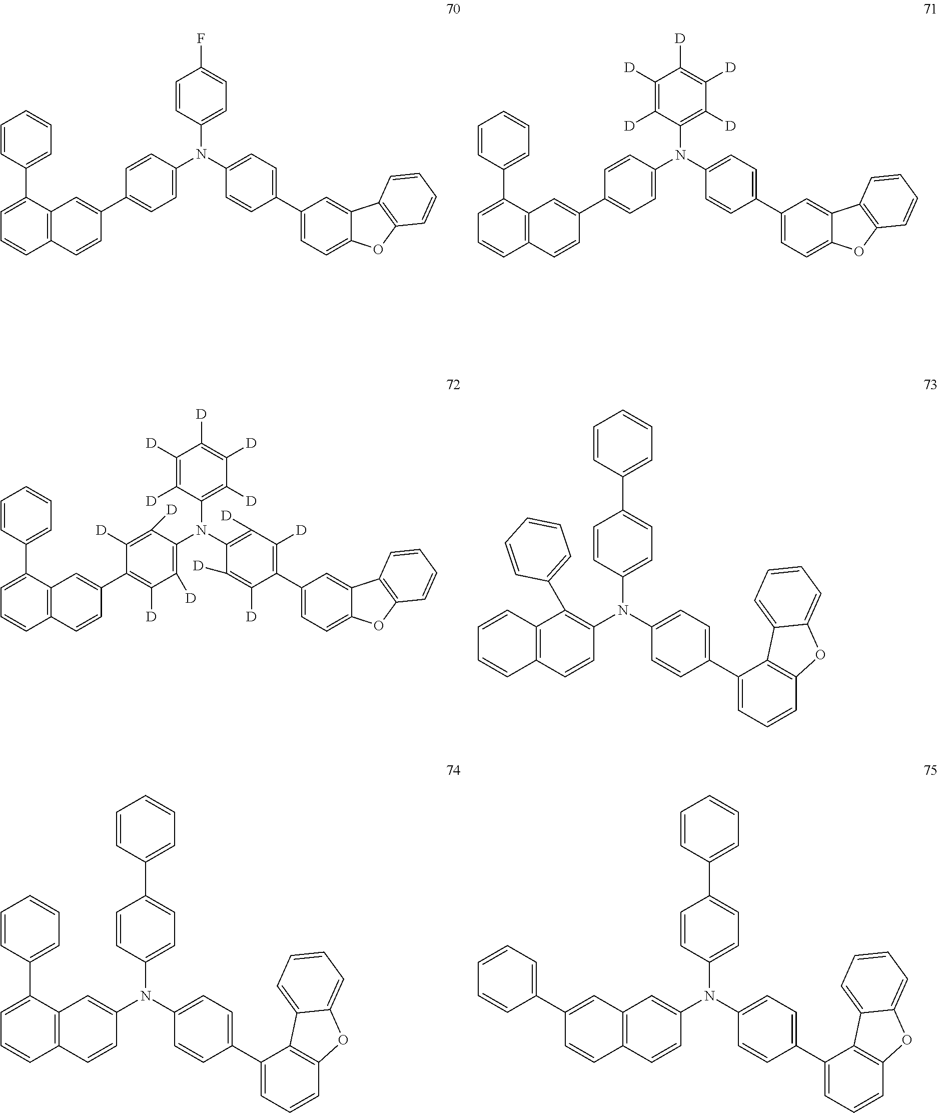 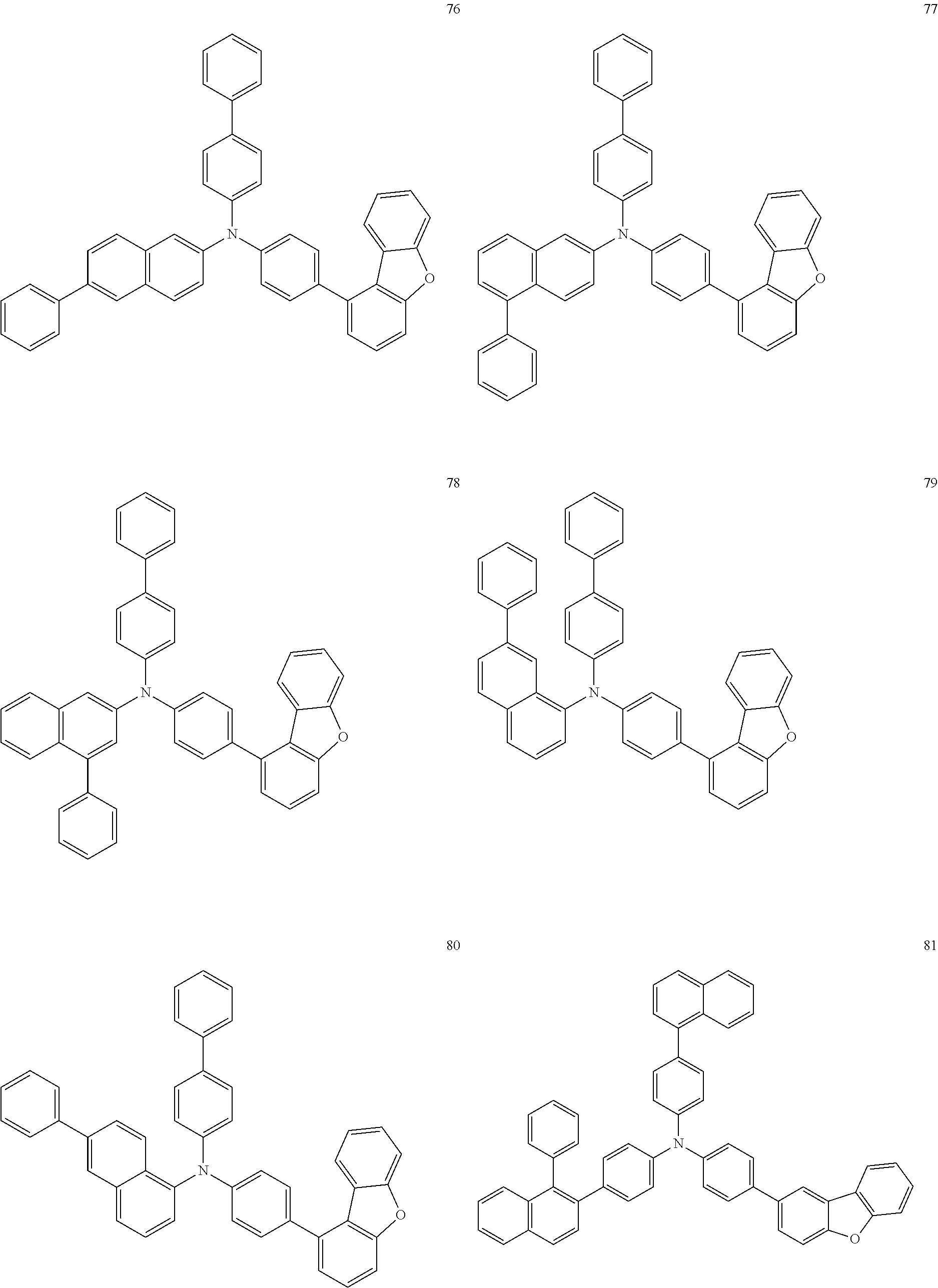 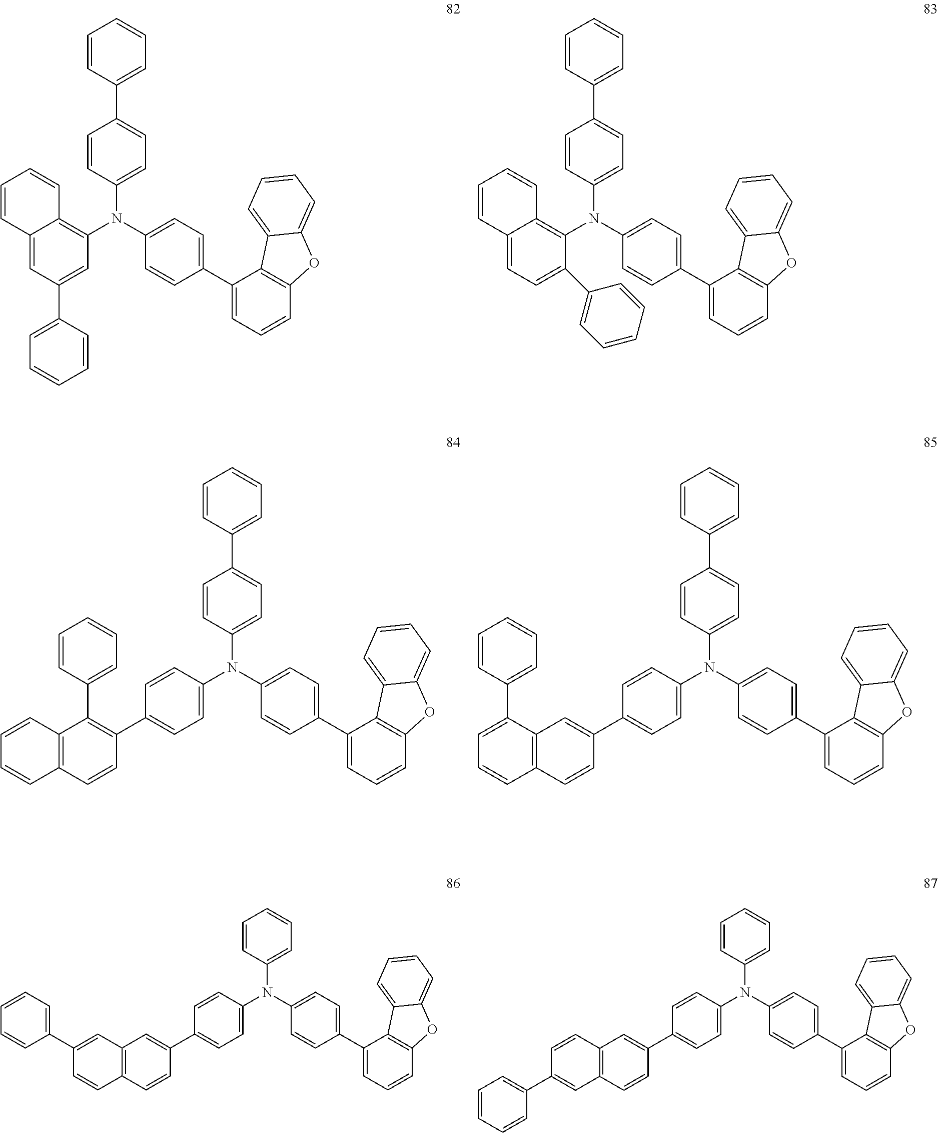 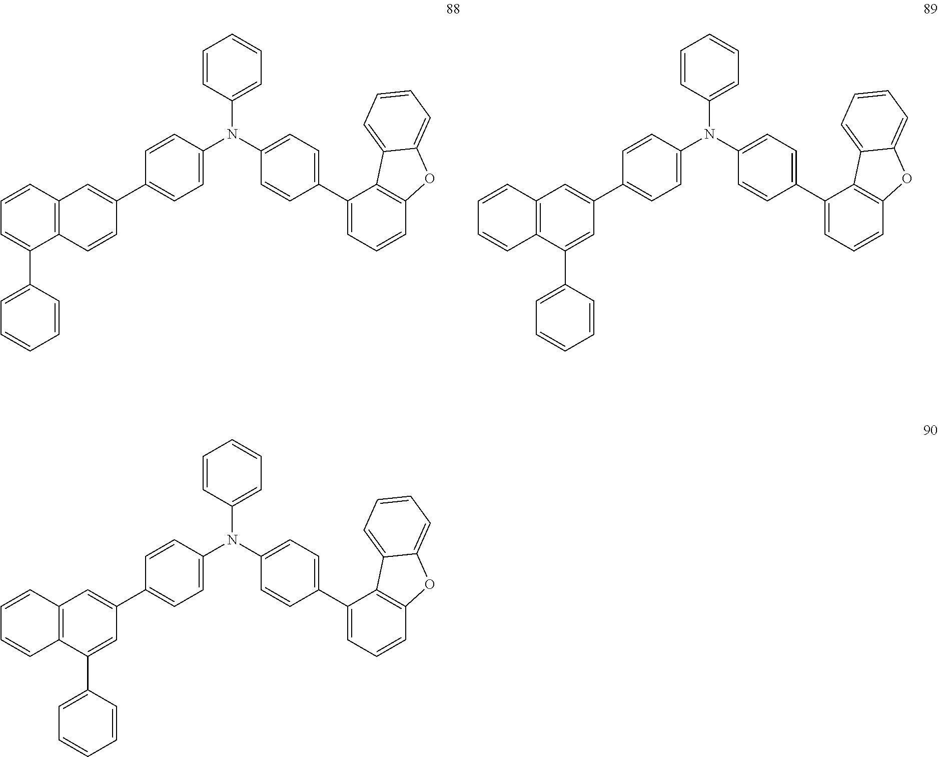 [SMILES: c1ccc(-c2cccc3cc(-c4ccc(N(c5ccccc5)c5ccc(-c6cccc7oc8ccccc8c67)cc5)cc4)ccc23)cc1 ; c1ccc(-c2cc(-c3ccc(N(c4ccccc4)c4ccc(-c5cccc6oc7ccccc7c56)cc4)cc3)cc3ccccc23)cc1 ; c1ccc(-c2cc(-c3ccc(N(c4ccccc4)c4ccc(-c5cccc6oc7ccccc7c56)cc4)cc3)cc3ccccc23)cc1] 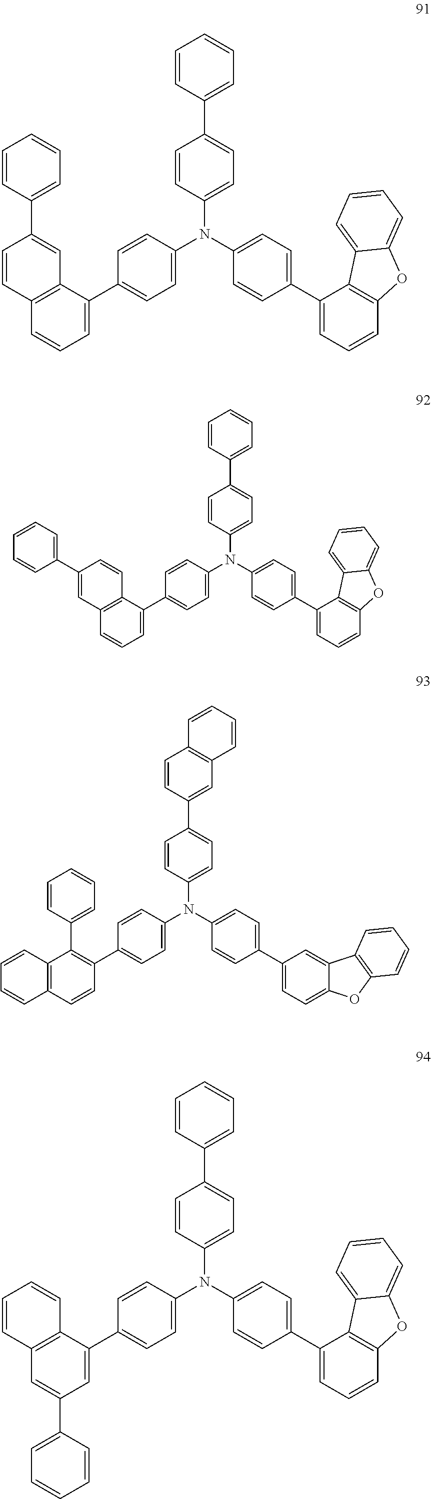 [SMILES: c1ccc(-c2ccc(N(c3ccc(-c4cccc5ccc(-c6ccccc6)cc45)cc3)c3ccc(-c4cccc5oc6ccccc6c45)cc3)cc2)cc1 ; c1ccc(-c2ccc(N(c3ccc(-c4cccc5cc(-c6ccccc6)ccc45)cc3)c3ccc(-c4cccc5oc6ccccc6c45)cc3)cc2)cc1 ; c1ccc(-c2c(-c3ccc(N(c4ccc(-c5ccc6ccccc6c5)cc4)c4ccc(-c5ccc6oc7ccccc7c6c5)cc4)cc3)ccc3ccccc23)cc1 ; c1ccc(-c2ccc(N(c3ccc(-c4cc(-c5ccccc5)cc5ccccc45)cc3)c3ccc(-c4cccc5oc6ccccc6c45)cc3)cc2)cc1] 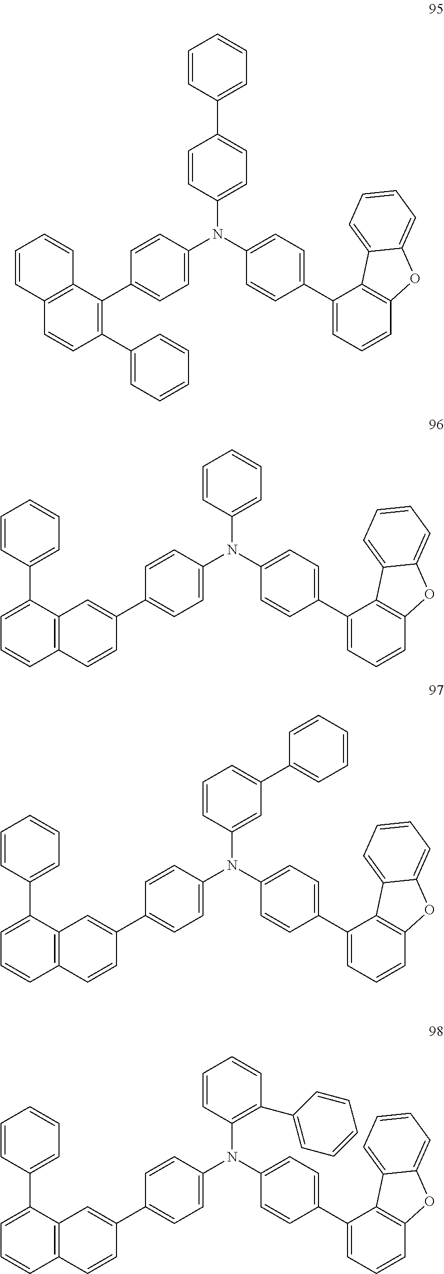 [SMILES: c1ccc(-c2ccc(N(c3ccc(-c4c(-c5ccccc5)ccc5ccccc45)cc3)c3ccc(-c4cccc5oc6ccccc6c45)cc3)cc2)cc1 ; c1ccc(-c2cccc3ccc(-c4ccc(N(c5ccccc5)c5ccc(-c6cccc7oc8ccccc8c67)cc5)cc4)cc23)cc1 ; c1ccc(-c2cccc(N(c3ccc(-c4ccc5cccc(-c6ccccc6)c5c4)cc3)c3ccc(-c4cccc5oc6ccccc6c45)cc3)c2)cc1 ; c1ccc(-c2ccccc2N(c2ccc(-c3ccc4cccc(-c5ccccc5)c4c3)cc2)c2ccc(-c3cccc4oc5ccccc5c34)cc2)cc1] 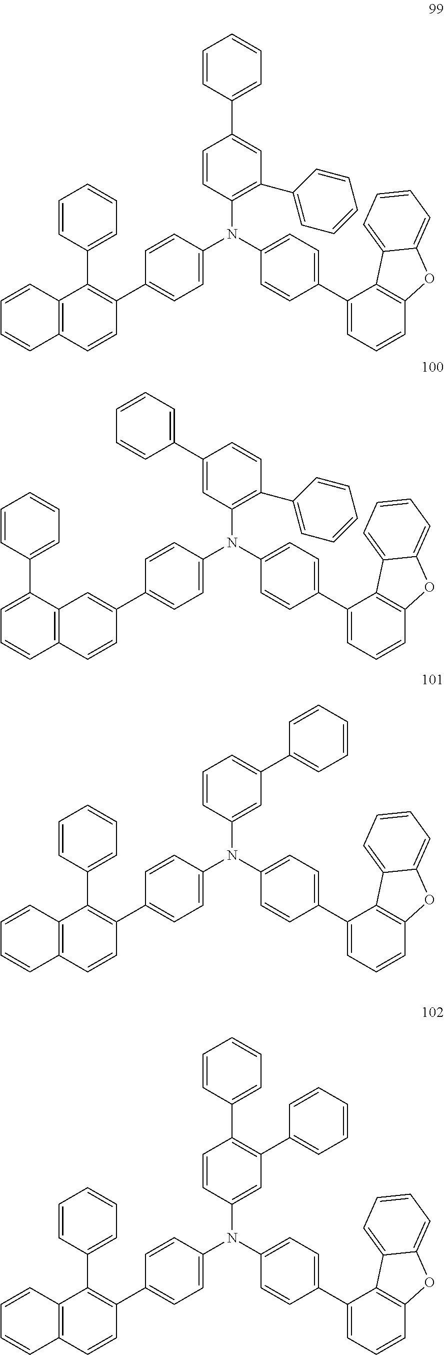 [SMILES: c1ccc(-c2ccc(N(c3ccc(-c4ccc5ccccc5c4-c4ccccc4)cc3)c3ccc(-c4cccc5oc6ccccc6c45)cc3)c(-c3ccccc3)c2)cc1 ; c1ccc(-c2ccc(-c3ccccc3)c(N(c3ccc(-c4ccc5cccc(-c6ccccc6)c5c4)cc3)c3ccc(-c4cccc5oc6ccccc6c45)cc3)c2)cc1 ; c1ccc(-c2cccc(N(c3ccc(-c4ccc5ccccc5c4-c4ccccc4)cc3)c3ccc(-c4cccc5oc6ccccc6c45)cc3)c2)cc1 ; c1ccc(-c2ccc(N(c3ccc(-c4ccc5ccccc5c4-c4ccccc4)cc3)c3ccc(-c4cccc5oc6ccccc6c45)cc3)cc2-c2ccccc2)cc1] 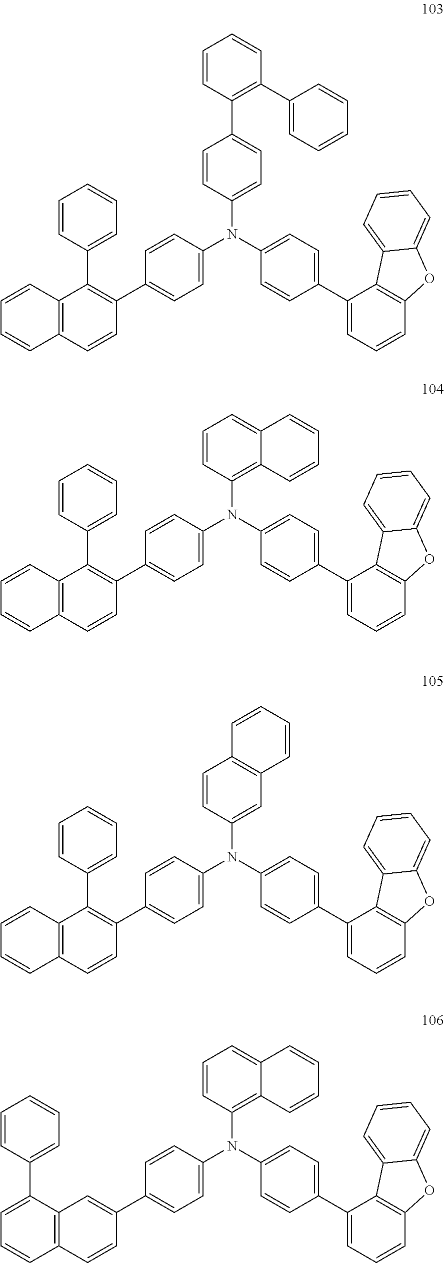 [SMILES: c1ccc(-c2ccccc2-c2ccc(N(c3ccc(-c4ccc5ccccc5c4-c4ccccc4)cc3)c3ccc(-c4cccc5oc6ccccc6c45)cc3)cc2)cc1 ; c1ccc(-c2c(-c3ccc(N(c4ccc(-c5cccc6oc7ccccc7c56)cc4)c4cccc5ccccc45)cc3)ccc3ccccc23)cc1 ; c1ccc(-c2c(-c3ccc(N(c4ccc(-c5cccc6oc7ccccc7c56)cc4)c4ccc5ccccc5c4)cc3)ccc3ccccc23)cc1 ; c1ccc(-c2cccc3ccc(-c4ccc(N(c5ccc(-c6cccc7oc8ccccc8c67)cc5)c5cccc6ccccc56)cc4)cc23)cc1] 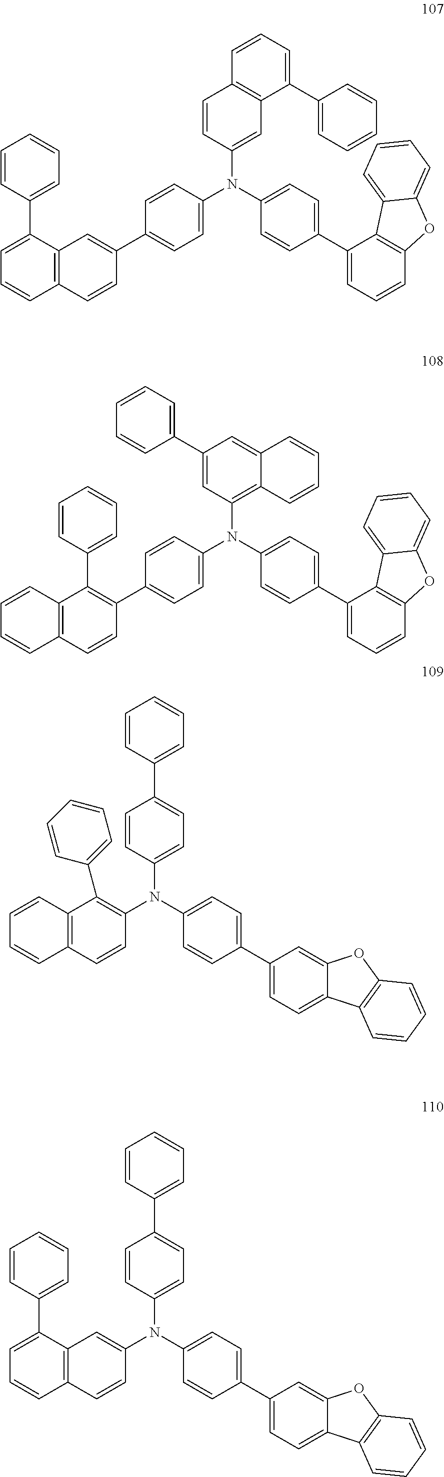 [SMILES: c1ccc(-c2cccc3ccc(-c4ccc(N(c5ccc(-c6cccc7oc8ccccc8c67)cc5)c5ccc6cccc(-c7ccccc7)c6c5)cc4)cc23)cc1 ; c1ccc(-c2cc(N(c3ccc(-c4ccc5ccccc5c4-c4ccccc4)cc3)c3ccc(-c4cccc5oc6ccccc6c45)cc3)c3ccccc3c2)cc1 ; c1ccc(-c2ccc(N(c3ccc(-c4ccc5c(c4)oc4ccccc45)cc3)c3ccc4ccccc4c3-c3ccccc3)cc2)cc1 ; c1ccc(-c2ccc(N(c3ccc(-c4ccc5c(c4)oc4ccccc45)cc3)c3ccc4cccc(-c5ccccc5)c4c3)cc2)cc1] 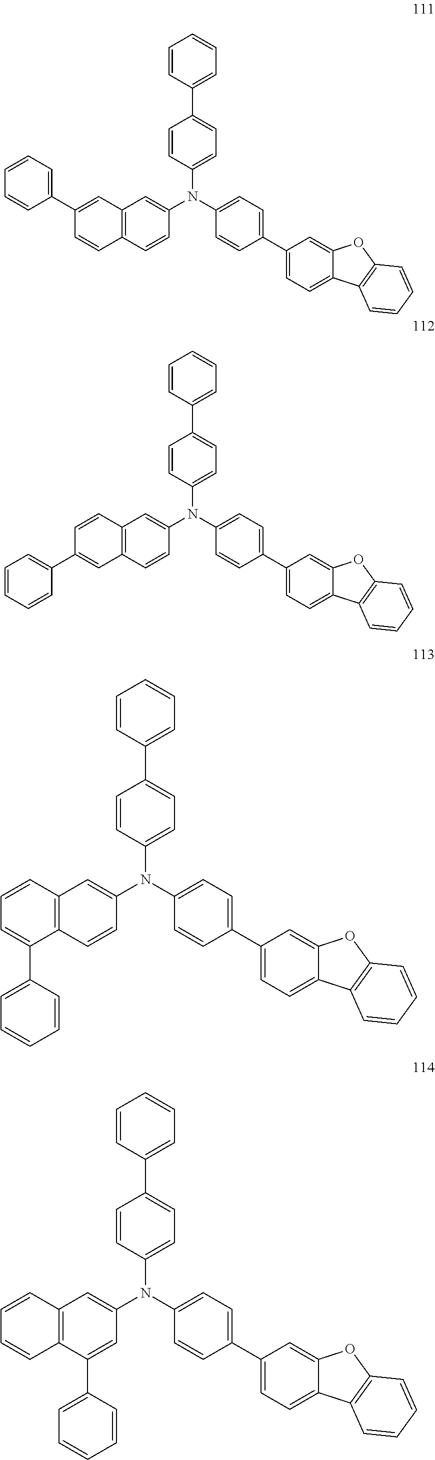 [SMILES: c1ccc(-c2ccc(N(c3ccc(-c4ccc5c(c4)oc4ccccc45)cc3)c3ccc4ccc(-c5ccccc5)cc4c3)cc2)cc1 ; c1ccc(-c2ccc(N(c3ccc(-c4ccc5c(c4)oc4ccccc45)cc3)c3ccc4cc(-c5ccccc5)ccc4c3)cc2)cc1 ; c1ccc(-c2ccc(N(c3ccc(-c4ccc5c(c4)oc4ccccc45)cc3)c3ccc4c(-c5ccccc5)cccc4c3)cc2)cc1 ; c1ccc(-c2ccc(N(c3ccc(-c4ccc5c(c4)oc4ccccc45)cc3)c3cc(-c4ccccc4)c4ccccc4c3)cc2)cc1] 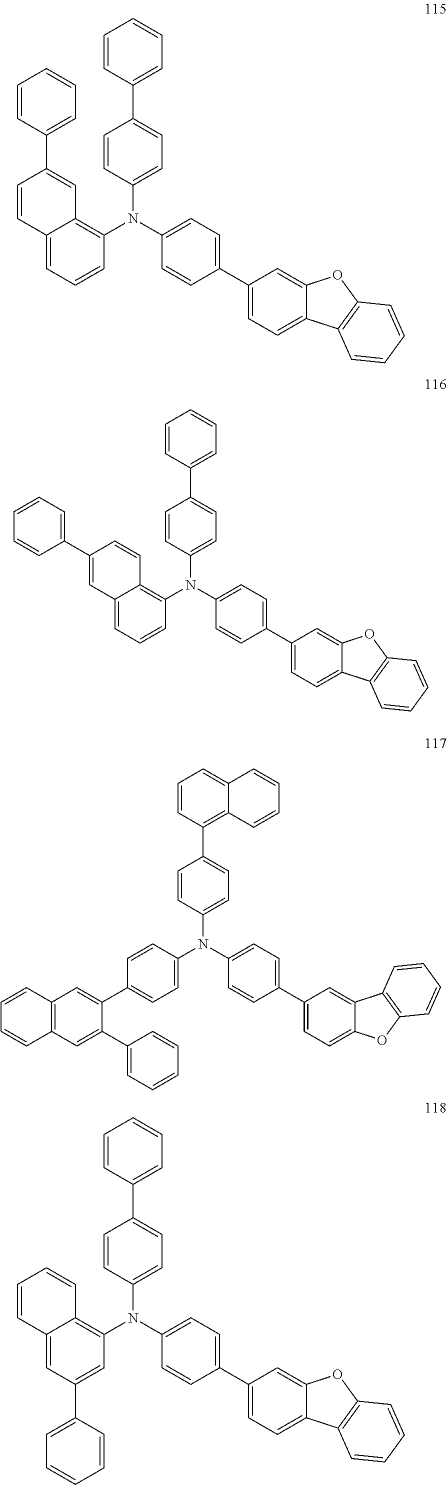 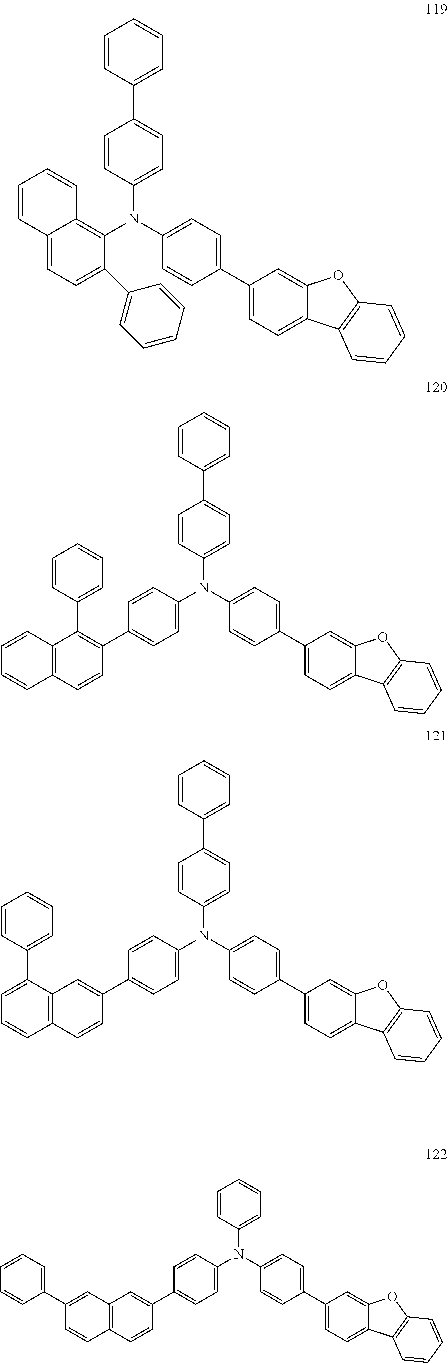 [SMILES: c1ccc(-c2ccc(N(c3ccc(-c4ccc5c(c4)oc4ccccc45)cc3)c3c(-c4ccccc4)ccc4ccccc34)cc2)cc1 ; c1ccc(-c2ccc(N(c3ccc(-c4ccc5c(c4)oc4ccccc45)cc3)c3ccc(-c4ccc5ccccc5c4-c4ccccc4)cc3)cc2)cc1 ; c1ccc(-c2ccc(N(c3ccc(-c4ccc5c(c4)oc4ccccc45)cc3)c3ccc(-c4ccc5cccc(-c6ccccc6)c5c4)cc3)cc2)cc1 ; c1ccc(-c2ccc3ccc(-c4ccc(N(c5ccccc5)c5ccc(-c6ccc7c(c6)oc6ccccc67)cc5)cc4)cc3c2)cc1] 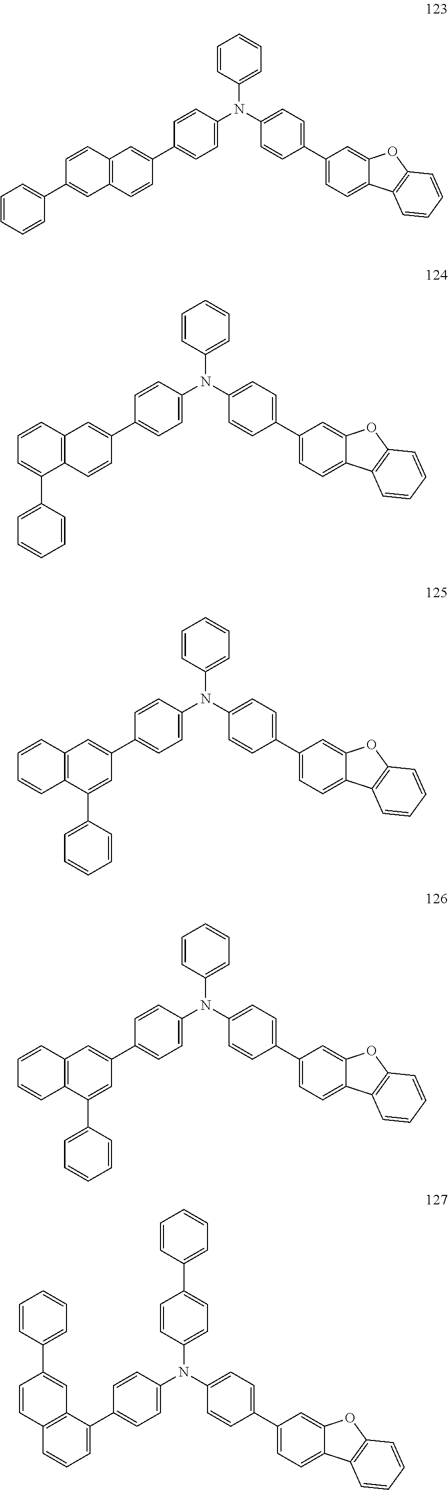 [SMILES: c1ccc(-c2ccc3cc(-c4ccc(N(c5ccccc5)c5ccc(-c6ccc7c(c6)oc6ccccc67)cc5)cc4)ccc3c2)cc1 ; c1ccc(-c2cccc3cc(-c4ccc(N(c5ccccc5)c5ccc(-c6ccc7c(c6)oc6ccccc67)cc5)cc4)ccc23)cc1 ; c1ccc(-c2cc(-c3ccc(N(c4ccccc4)c4ccc(-c5ccc6c(c5)oc5ccccc56)cc4)cc3)cc3ccccc23)cc1 ; c1ccc(-c2cc(-c3ccc(N(c4ccccc4)c4ccc(-c5ccc6c(c5)oc5ccccc56)cc4)cc3)cc3ccccc23)cc1 ; c1ccc(-c2ccc(N(c3ccc(-c4ccc5c(c4)oc4ccccc45)cc3)c3ccc(-c4cccc5ccc(-c6ccccc6)cc45)cc3)cc2)cc1] 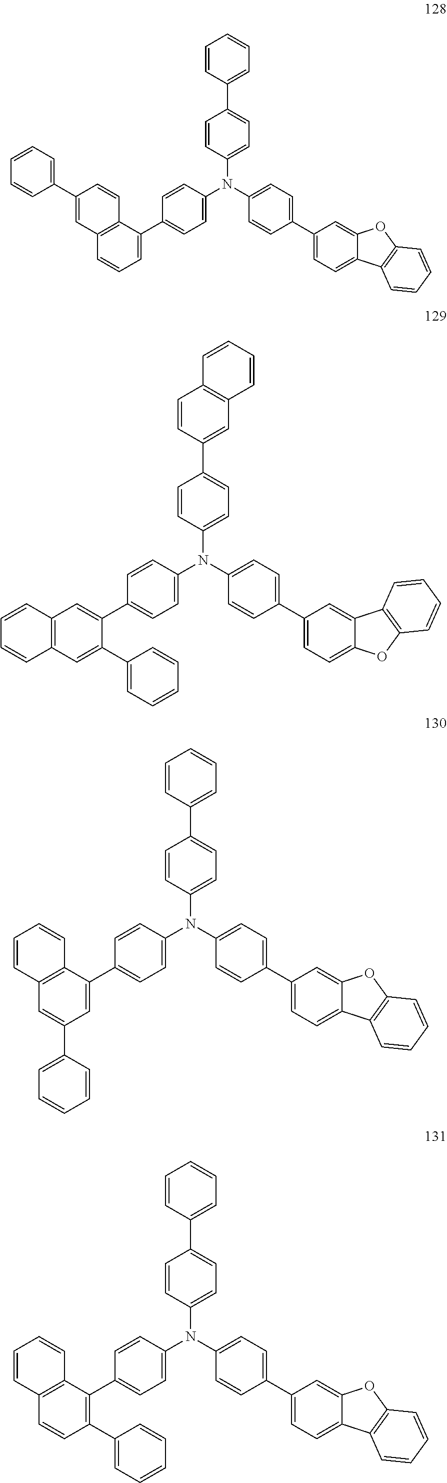 [SMILES: c1ccc(-c2ccc(N(c3ccc(-c4ccc5c(c4)oc4ccccc45)cc3)c3ccc(-c4cccc5cc(-c6ccccc6)ccc45)cc3)cc2)cc1 ; c1ccc(-c2cc3ccccc3cc2-c2ccc(N(c3ccc(-c4ccc5ccccc5c4)cc3)c3ccc(-c4ccc5oc6ccccc6c5c4)cc3)cc2)cc1 ; c1ccc(-c2ccc(N(c3ccc(-c4ccc5c(c4)oc4ccccc45)cc3)c3ccc(-c4cc(-c5ccccc5)cc5ccccc45)cc3)cc2)cc1 ; c1ccc(-c2ccc(N(c3ccc(-c4ccc5c(c4)oc4ccccc45)cc3)c3ccc(-c4c(-c5ccccc5)ccc5ccccc45)cc3)cc2)cc1] 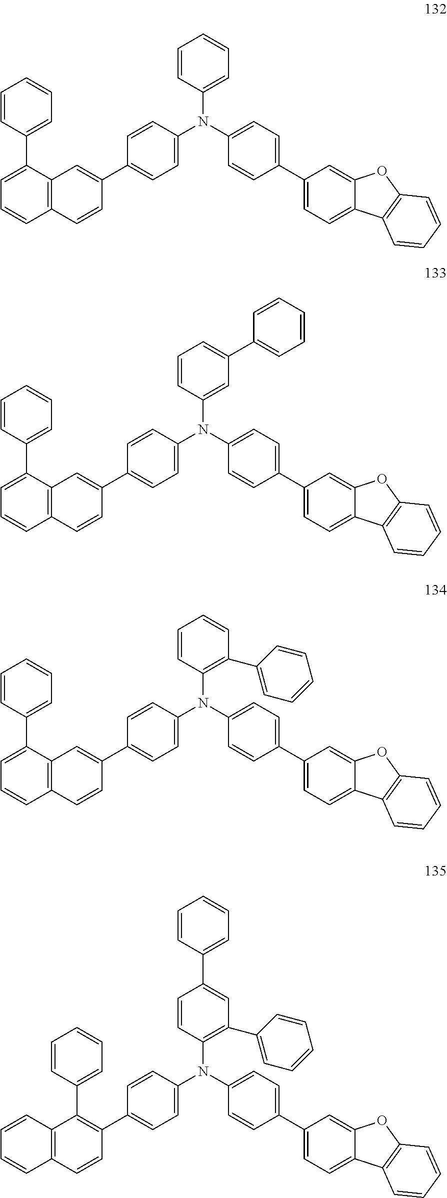 [SMILES: c1ccc(-c2cccc3ccc(-c4ccc(N(c5ccccc5)c5ccc(-c6ccc7c(c6)oc6ccccc67)cc5)cc4)cc23)cc1 ; c1ccc(-c2cccc(N(c3ccc(-c4ccc5c(c4)oc4ccccc45)cc3)c3ccc(-c4ccc5cccc(-c6ccccc6)c5c4)cc3)c2)cc1 ; c1ccc(-c2ccccc2N(c2ccc(-c3ccc4c(c3)oc3ccccc34)cc2)c2ccc(-c3ccc4cccc(-c5ccccc5)c4c3)cc2)cc1 ; c1ccc(-c2ccc(N(c3ccc(-c4ccc5c(c4)oc4ccccc45)cc3)c3ccc(-c4ccc5ccccc5c4-c4ccccc4)cc3)c(-c3ccccc3)c2)cc1] 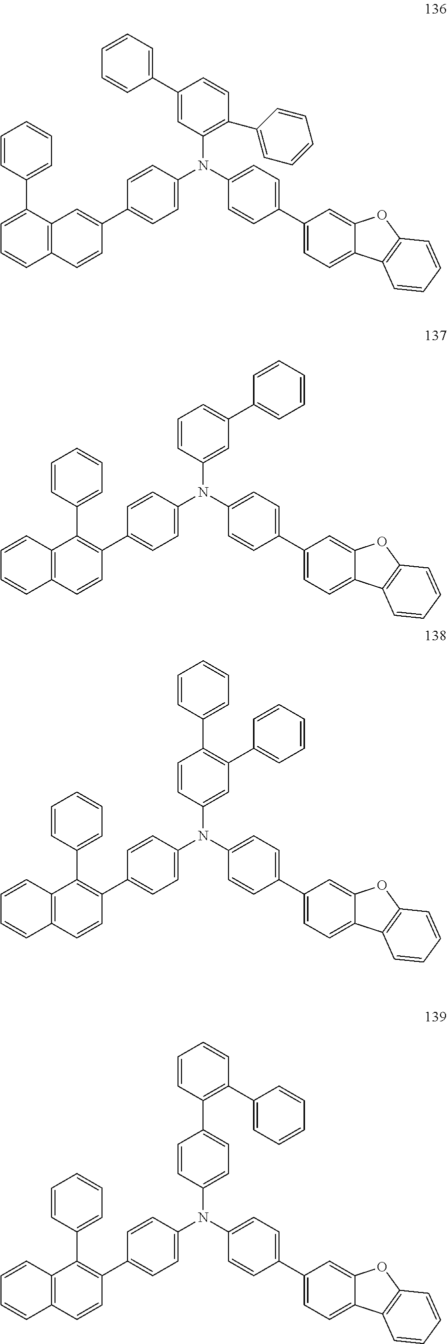 [SMILES: c1ccc(-c2ccc(-c3ccccc3)c(N(c3ccc(-c4ccc5c(c4)oc4ccccc45)cc3)c3ccc(-c4ccc5cccc(-c6ccccc6)c5c4)cc3)c2)cc1 ; c1ccc(-c2cccc(N(c3ccc(-c4ccc5c(c4)oc4ccccc45)cc3)c3ccc(-c4ccc5ccccc5c4-c4ccccc4)cc3)c2)cc1 ; c1ccc(-c2ccc(N(c3ccc(-c4ccc5c(c4)oc4ccccc45)cc3)c3ccc(-c4ccc5ccccc5c4-c4ccccc4)cc3)cc2-c2ccccc2)cc1 ; c1ccc(-c2ccccc2-c2ccc(N(c3ccc(-c4ccc5c(c4)oc4ccccc45)cc3)c3ccc(-c4ccc5ccccc5c4-c4ccccc4)cc3)cc2)cc1] 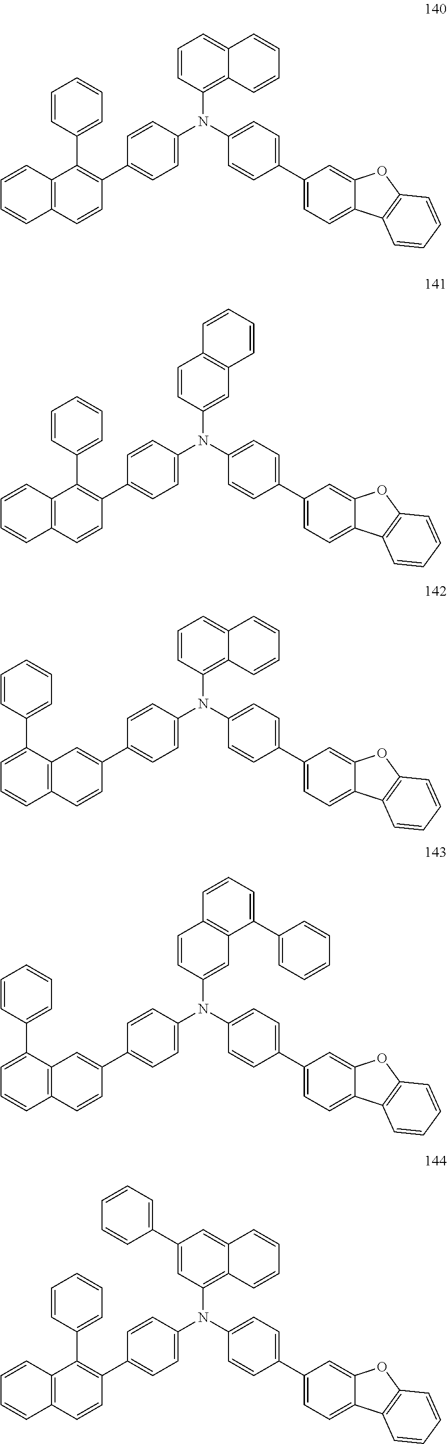 [SMILES: c1ccc(-c2c(-c3ccc(N(c4ccc(-c5ccc6c(c5)oc5ccccc56)cc4)c4cccc5ccccc45)cc3)ccc3ccccc23)cc1 ; c1ccc(-c2c(-c3ccc(N(c4ccc(-c5ccc6c(c5)oc5ccccc56)cc4)c4ccc5ccccc5c4)cc3)ccc3ccccc23)cc1 ; c1ccc(-c2cccc3ccc(-c4ccc(N(c5ccc(-c6ccc7c(c6)oc6ccccc67)cc5)c5cccc6ccccc56)cc4)cc23)cc1 ; c1ccc(-c2cccc3ccc(-c4ccc(N(c5ccc(-c6ccc7c(c6)oc6ccccc67)cc5)c5ccc6cccc(-c7ccccc7)c6c5)cc4)cc23)cc1 ; c1ccc(-c2cc(N(c3ccc(-c4ccc5c(c4)oc4ccccc45)cc3)c3ccc(-c4ccc5ccccc5c4-c4ccccc4)cc3)c3ccccc3c2)cc1] 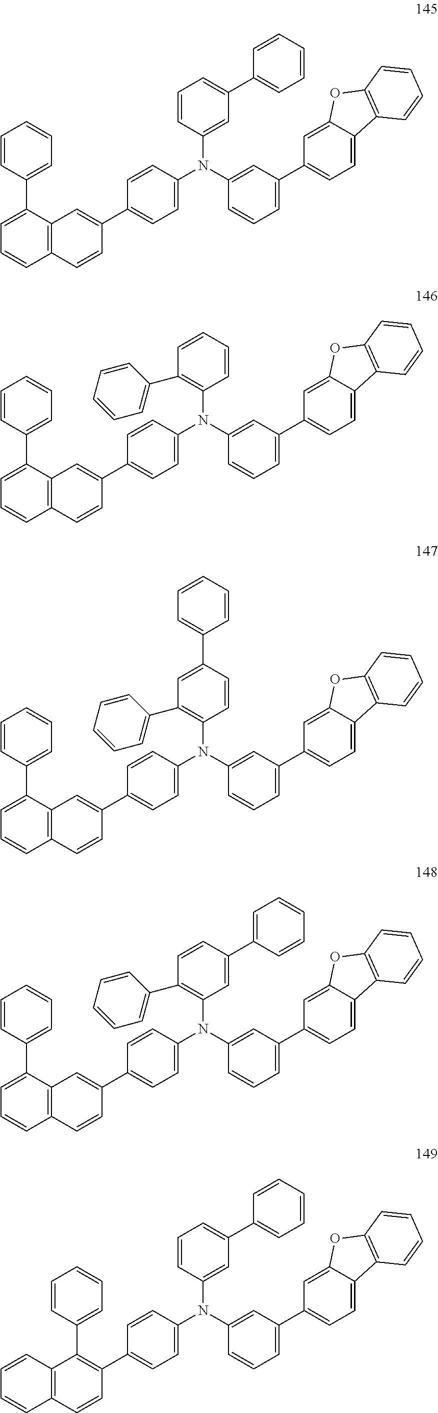 [SMILES: c1ccc(-c2cccc(N(c3ccc(-c4ccc5cccc(-c6ccccc6)c5c4)cc3)c3cccc(-c4ccc5c(c4)oc4ccccc45)c3)c2)cc1 ; c1ccc(-c2ccccc2N(c2ccc(-c3ccc4cccc(-c5ccccc5)c4c3)cc2)c2cccc(-c3ccc4c(c3)oc3ccccc34)c2)cc1 ; c1ccc(-c2ccc(N(c3ccc(-c4ccc5cccc(-c6ccccc6)c5c4)cc3)c3cccc(-c4ccc5c(c4)oc4ccccc45)c3)c(-c3ccccc3)c2)cc1 ; c1ccc(-c2ccc(-c3ccccc3)c(N(c3ccc(-c4ccc5cccc(-c6ccccc6)c5c4)cc3)c3cccc(-c4ccc5c(c4)oc4ccccc45)c3)c2)cc1 ; c1ccc(-c2cccc(N(c3ccc(-c4ccc5ccccc5c4-c4ccccc4)cc3)c3cccc(-c4ccc5c(c4)oc4ccccc45)c3)c2)cc1] 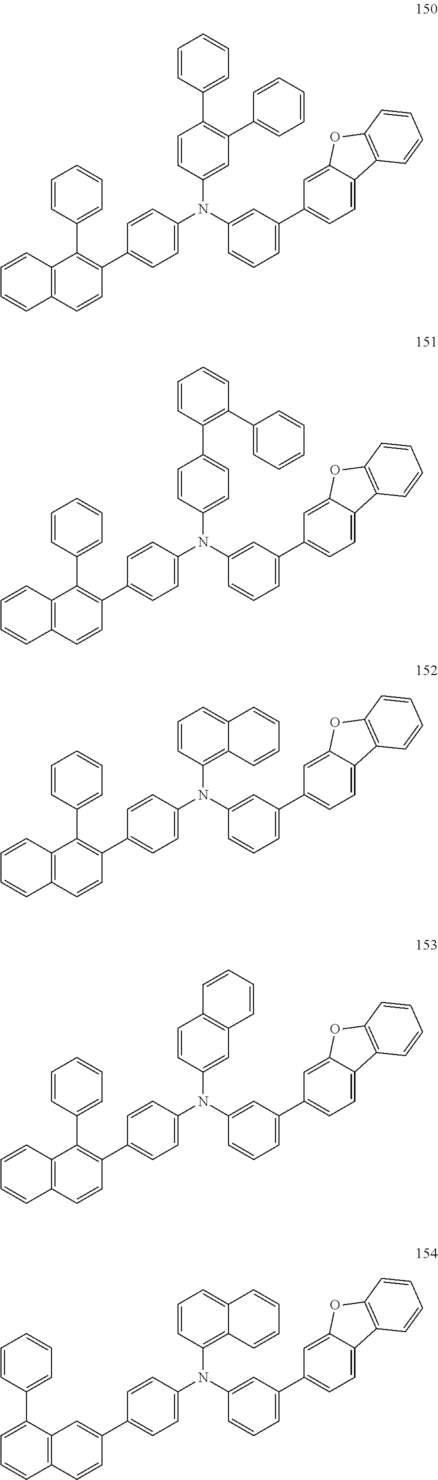 [SMILES: c1ccc(-c2ccc(N(c3ccc(-c4ccc5ccccc5c4-c4ccccc4)cc3)c3cccc(-c4ccc5c(c4)oc4ccccc45)c3)cc2-c2ccccc2)cc1 ; c1ccc(-c2ccccc2-c2ccc(N(c3ccc(-c4ccc5ccccc5c4-c4ccccc4)cc3)c3cccc(-c4ccc5c(c4)oc4ccccc45)c3)cc2)cc1 ; c1ccc(-c2c(-c3ccc(N(c4cccc(-c5ccc6c(c5)oc5ccccc56)c4)c4cccc5ccccc45)cc3)ccc3ccccc23)cc1 ; c1ccc(-c2c(-c3ccc(N(c4cccc(-c5ccc6c(c5)oc5ccccc56)c4)c4ccc5ccccc5c4)cc3)ccc3ccccc23)cc1 ; c1ccc(-c2cccc3ccc(-c4ccc(N(c5cccc(-c6ccc7c(c6)oc6ccccc67)c5)c5cccc6ccccc56)cc4)cc23)cc1] 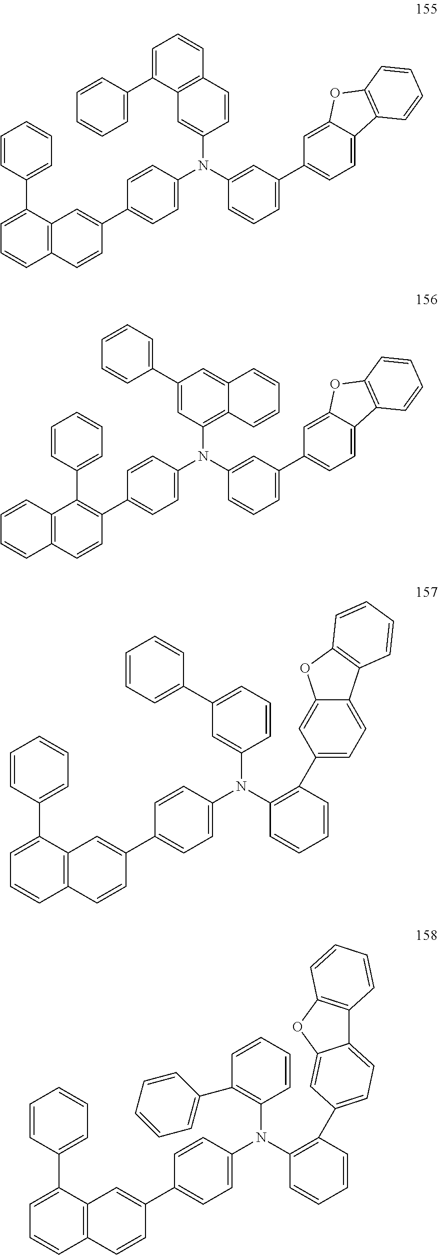 [SMILES: c1ccc(-c2cccc3ccc(-c4ccc(N(c5cccc(-c6ccc7c(c6)oc6ccccc67)c5)c5ccc6cccc(-c7ccccc7)c6c5)cc4)cc23)cc1 ; c1ccc(-c2cc(N(c3ccc(-c4ccc5ccccc5c4-c4ccccc4)cc3)c3cccc(-c4ccc5c(c4)oc4ccccc45)c3)c3ccccc3c2)cc1 ; c1ccc(-c2cccc(N(c3ccc(-c4ccc5cccc(-c6ccccc6)c5c4)cc3)c3ccccc3-c3ccc4c(c3)oc3ccccc34)c2)cc1 ; c1ccc(-c2ccccc2N(c2ccc(-c3ccc4cccc(-c5ccccc5)c4c3)cc2)c2ccccc2-c2ccc3c(c2)oc2ccccc23)cc1] 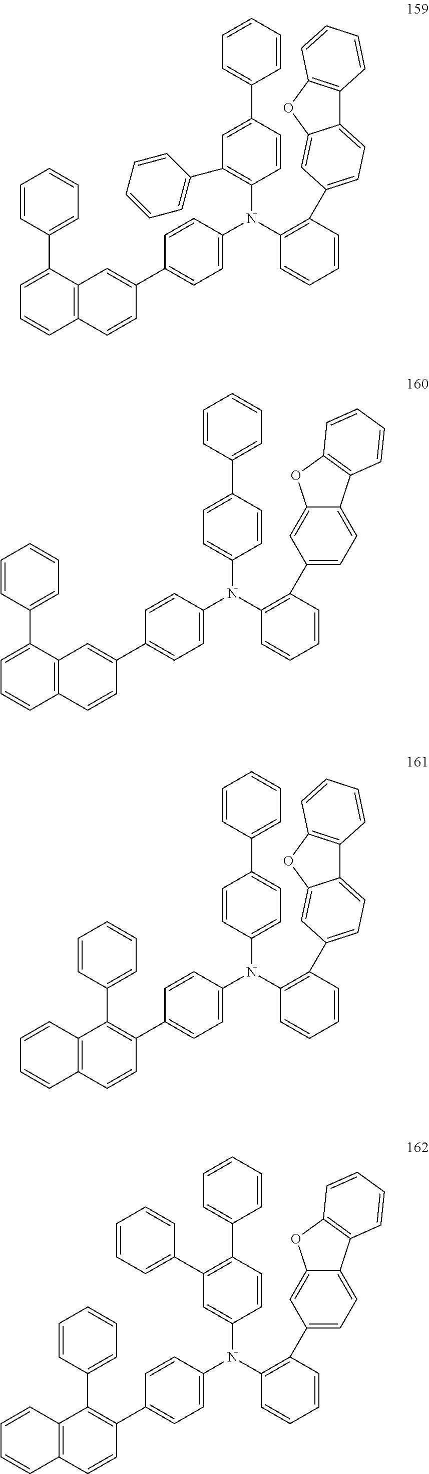 [SMILES: c1ccc(-c2ccc(N(c3ccc(-c4ccc5cccc(-c6ccccc6)c5c4)cc3)c3ccccc3-c3ccc4c(c3)oc3ccccc34)c(-c3ccccc3)c2)cc1 ; c1ccc(-c2ccc(N(c3ccc(-c4ccc5cccc(-c6ccccc6)c5c4)cc3)c3ccccc3-c3ccc4c(c3)oc3ccccc34)cc2)cc1 ; c1ccc(-c2ccc(N(c3ccc(-c4ccc5ccccc5c4-c4ccccc4)cc3)c3ccccc3-c3ccc4c(c3)oc3ccccc34)cc2)cc1 ; c1ccc(-c2ccc(N(c3ccc(-c4ccc5ccccc5c4-c4ccccc4)cc3)c3ccccc3-c3ccc4c(c3)oc3ccccc34)cc2-c2ccccc2)cc1] 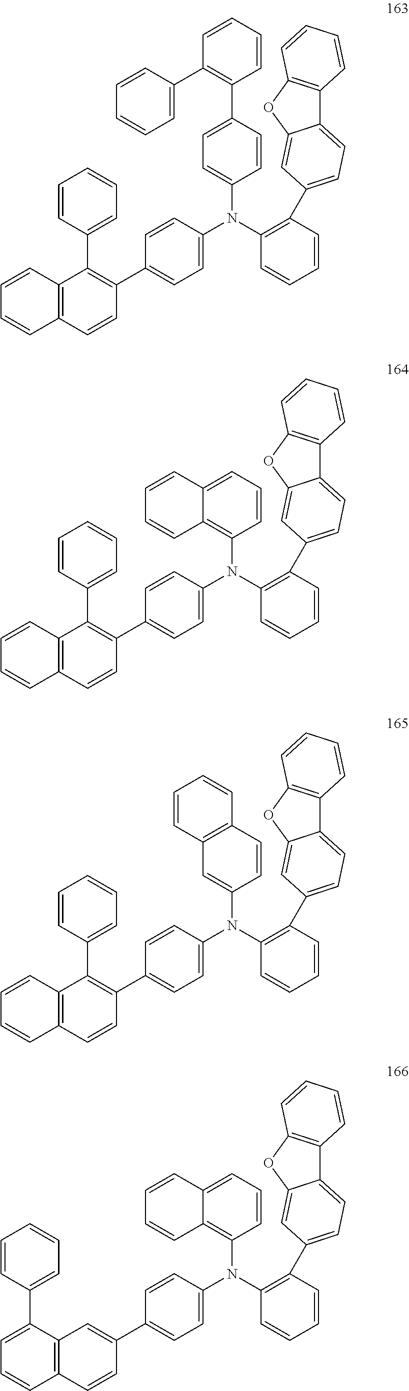 [SMILES: c1ccc(-c2ccccc2-c2ccc(N(c3ccc(-c4ccc5ccccc5c4-c4ccccc4)cc3)c3ccccc3-c3ccc4c(c3)oc3ccccc34)cc2)cc1 ; c1ccc(-c2c(-c3ccc(N(c4ccccc4-c4ccc5c(c4)oc4ccccc45)c4cccc5ccccc45)cc3)ccc3ccccc23)cc1 ; c1ccc(-c2c(-c3ccc(N(c4ccc5ccccc5c4)c4ccccc4-c4ccc5c(c4)oc4ccccc45)cc3)ccc3ccccc23)cc1 ; c1ccc(-c2cccc3ccc(-c4ccc(N(c5ccccc5-c5ccc6c(c5)oc5ccccc56)c5cccc6ccccc56)cc4)cc23)cc1] 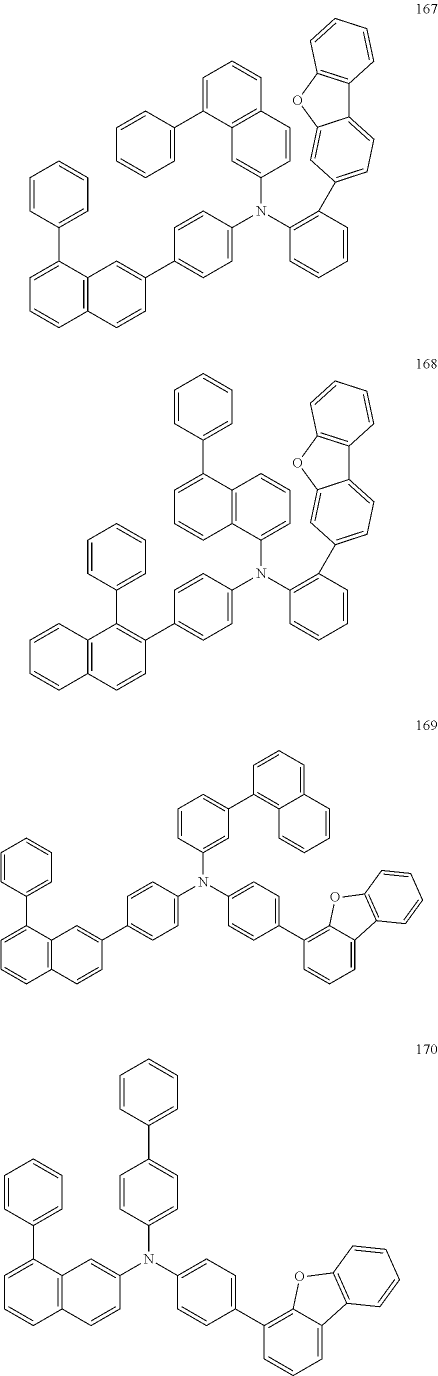 [SMILES: c1ccc(-c2cccc3ccc(-c4ccc(N(c5ccc6cccc(-c7ccccc7)c6c5)c5ccccc5-c5ccc6c(c5)oc5ccccc56)cc4)cc23)cc1 ; c1ccc(-c2c(-c3ccc(N(c4ccccc4-c4ccc5c(c4)oc4ccccc45)c4cccc5c(-c6ccccc6)cccc45)cc3)ccc3ccccc23)cc1 ; c1ccc(-c2cccc3ccc(-c4ccc(N(c5ccc(-c6cccc7c6oc6ccccc67)cc5)c5cccc(-c6cccc7ccccc67)c5)cc4)cc23)cc1 ; c1ccc(-c2ccc(N(c3ccc(-c4cccc5c4oc4ccccc45)cc3)c3ccc4cccc(-c5ccccc5)c4c3)cc2)cc1] 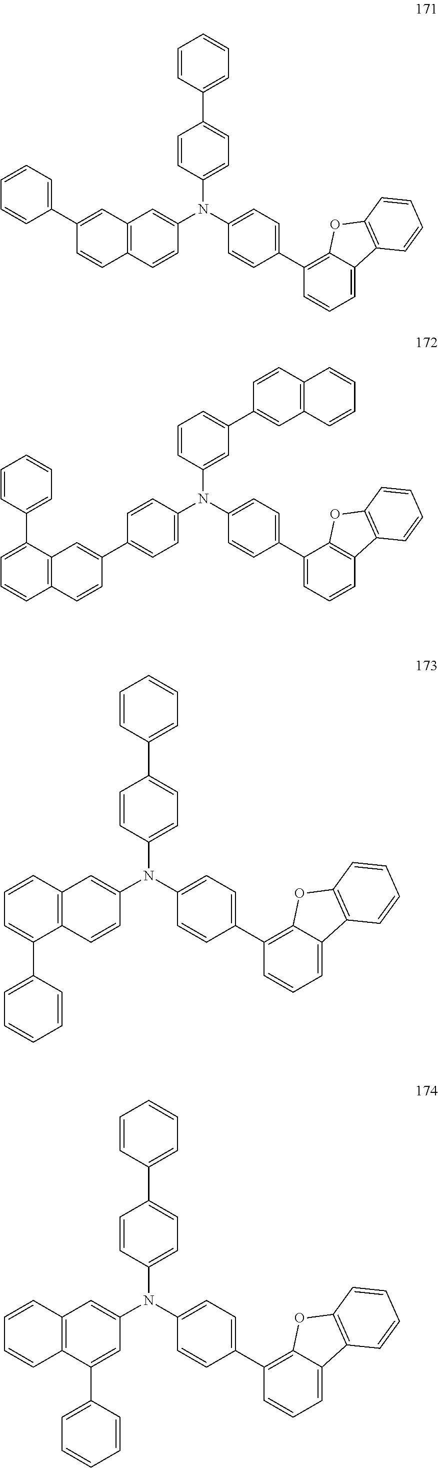 [SMILES: c1ccc(-c2ccc(N(c3ccc(-c4cccc5c4oc4ccccc45)cc3)c3ccc4ccc(-c5ccccc5)cc4c3)cc2)cc1 ; c1ccc(-c2cccc3ccc(-c4ccc(N(c5ccc(-c6cccc7c6oc6ccccc67)cc5)c5cccc(-c6ccc7ccccc7c6)c5)cc4)cc23)cc1 ; c1ccc(-c2ccc(N(c3ccc(-c4cccc5c4oc4ccccc45)cc3)c3ccc4c(-c5ccccc5)cccc4c3)cc2)cc1 ; c1ccc(-c2ccc(N(c3ccc(-c4cccc5c4oc4ccccc45)cc3)c3cc(-c4ccccc4)c4ccccc4c3)cc2)cc1] 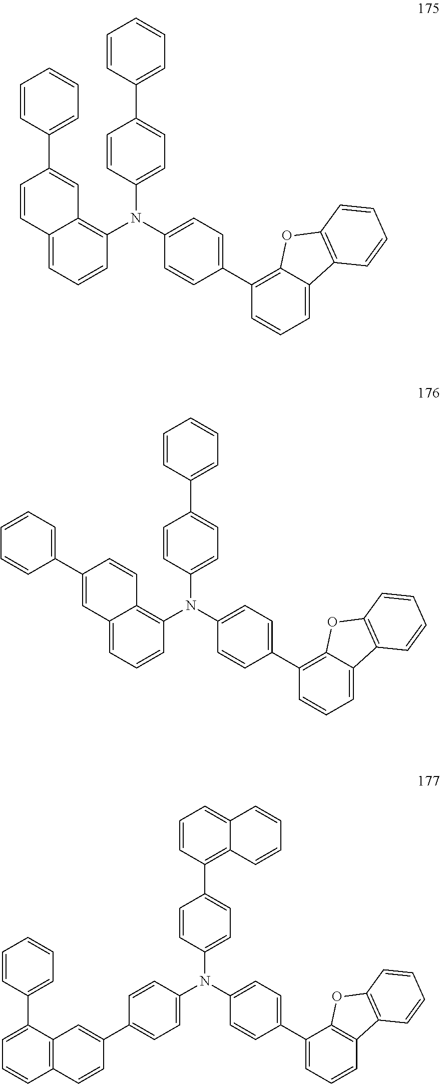 [SMILES: c1ccc(-c2ccc(N(c3ccc(-c4cccc5c4oc4ccccc45)cc3)c3cccc4ccc(-c5ccccc5)cc34)cc2)cc1 ; c1ccc(-c2ccc(N(c3ccc(-c4cccc5c4oc4ccccc45)cc3)c3cccc4cc(-c5ccccc5)ccc34)cc2)cc1 ; c1ccc(-c2cccc3ccc(-c4ccc(N(c5ccc(-c6cccc7ccccc67)cc5)c5ccc(-c6cccc7c6oc6ccccc67)cc5)cc4)cc23)cc1] 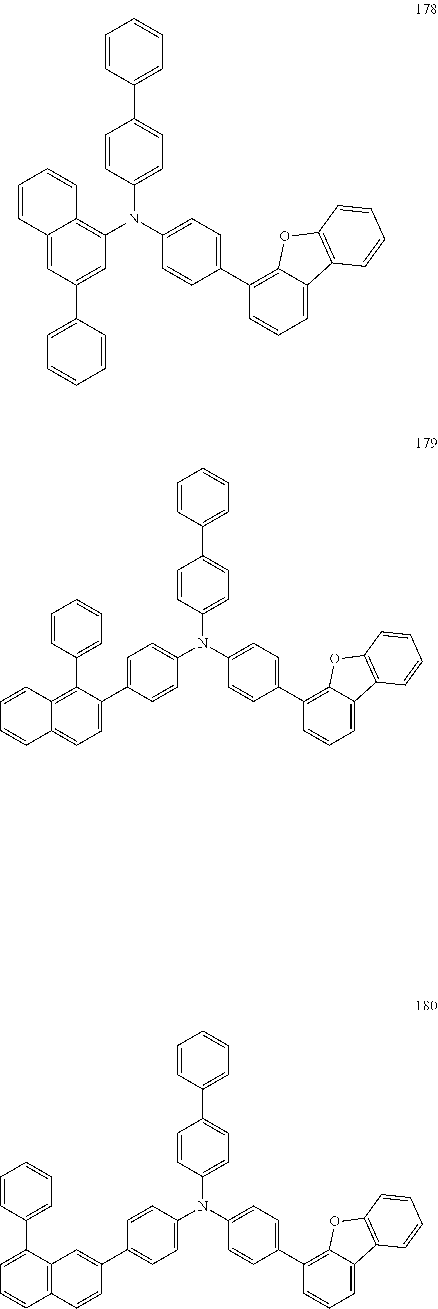 [SMILES: c1ccc(-c2ccc(N(c3ccc(-c4cccc5c4oc4ccccc45)cc3)c3cc(-c4ccccc4)cc4ccccc34)cc2)cc1 ; c1ccc(-c2ccc(N(c3ccc(-c4ccc5ccccc5c4-c4ccccc4)cc3)c3ccc(-c4cccc5c4oc4ccccc45)cc3)cc2)cc1 ; c1ccc(-c2ccc(N(c3ccc(-c4ccc5cccc(-c6ccccc6)c5c4)cc3)c3ccc(-c4cccc5c4oc4ccccc45)cc3)cc2)cc1] 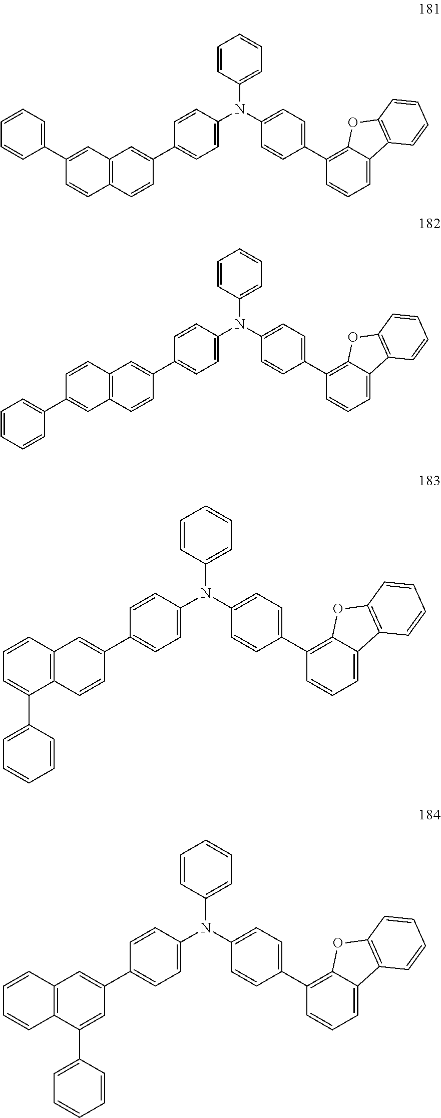 [SMILES: c1ccc(-c2ccc3ccc(-c4ccc(N(c5ccccc5)c5ccc(-c6cccc7c6oc6ccccc67)cc5)cc4)cc3c2)cc1 ; c1ccc(-c2ccc3cc(-c4ccc(N(c5ccccc5)c5ccc(-c6cccc7c6oc6ccccc67)cc5)cc4)ccc3c2)cc1 ; c1ccc(-c2cccc3cc(-c4ccc(N(c5ccccc5)c5ccc(-c6cccc7c6oc6ccccc67)cc5)cc4)ccc23)cc1 ; c1ccc(-c2cc(-c3ccc(N(c4ccccc4)c4ccc(-c5cccc6c5oc5ccccc56)cc4)cc3)cc3ccccc23)cc1] 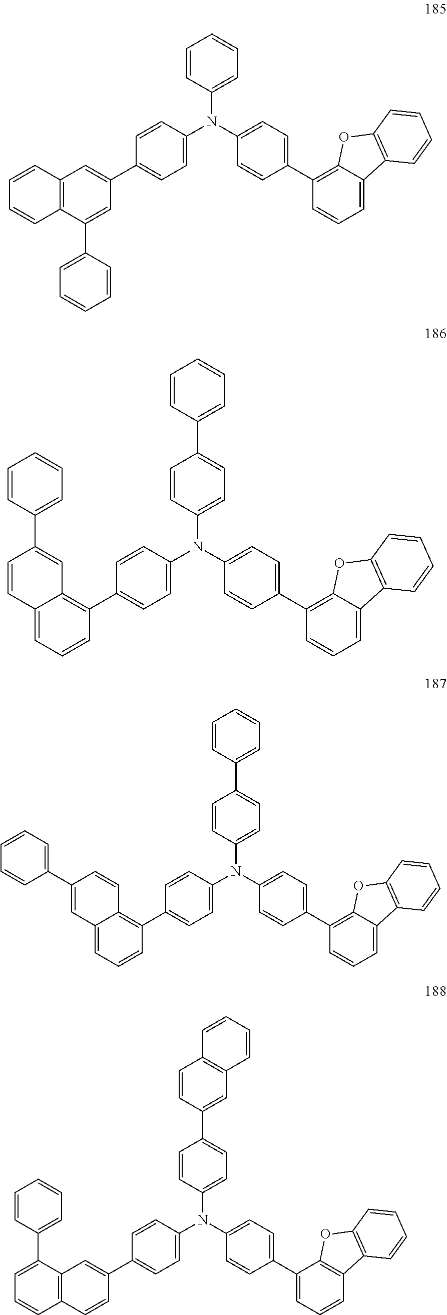 [SMILES: c1ccc(-c2cc(-c3ccc(N(c4ccccc4)c4ccc(-c5cccc6c5oc5ccccc56)cc4)cc3)cc3ccccc23)cc1 ; c1ccc(-c2ccc(N(c3ccc(-c4cccc5ccc(-c6ccccc6)cc45)cc3)c3ccc(-c4cccc5c4oc4ccccc45)cc3)cc2)cc1 ; c1ccc(-c2ccc(N(c3ccc(-c4cccc5cc(-c6ccccc6)ccc45)cc3)c3ccc(-c4cccc5c4oc4ccccc45)cc3)cc2)cc1 ; c1ccc(-c2cccc3ccc(-c4ccc(N(c5ccc(-c6ccc7ccccc7c6)cc5)c5ccc(-c6cccc7c6oc6ccccc67)cc5)cc4)cc23)cc1] 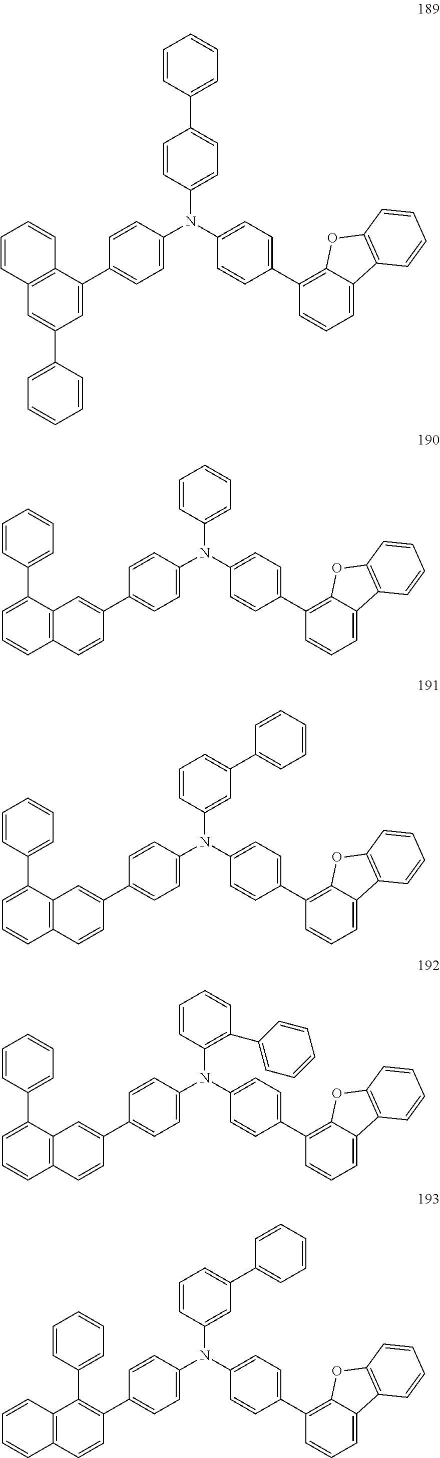 [SMILES: c1ccc(-c2ccc(N(c3ccc(-c4cc(-c5ccccc5)cc5ccccc45)cc3)c3ccc(-c4cccc5c4oc4ccccc45)cc3)cc2)cc1 ; c1ccc(-c2cccc3ccc(-c4ccc(N(c5ccccc5)c5ccc(-c6cccc7c6oc6ccccc67)cc5)cc4)cc23)cc1 ; c1ccc(-c2cccc(N(c3ccc(-c4ccc5cccc(-c6ccccc6)c5c4)cc3)c3ccc(-c4cccc5c4oc4ccccc45)cc3)c2)cc1 ; c1ccc(-c2ccccc2N(c2ccc(-c3ccc4cccc(-c5ccccc5)c4c3)cc2)c2ccc(-c3cccc4c3oc3ccccc34)cc2)cc1 ; c1ccc(-c2cccc(N(c3ccc(-c4ccc5ccccc5c4-c4ccccc4)cc3)c3ccc(-c4cccc5c4oc4ccccc45)cc3)c2)cc1] 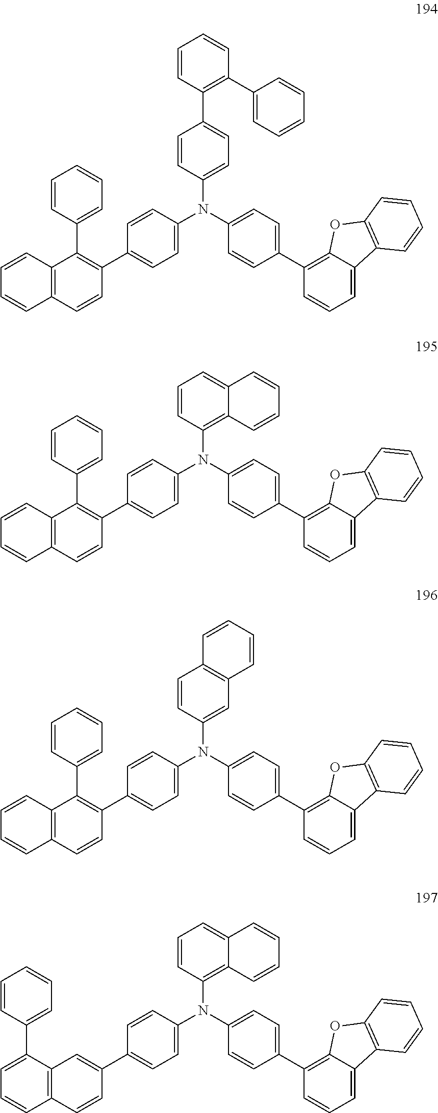 [SMILES: c1ccc(-c2ccccc2-c2ccc(N(c3ccc(-c4ccc5ccccc5c4-c4ccccc4)cc3)c3ccc(-c4cccc5c4oc4ccccc45)cc3)cc2)cc1 ; c1ccc(-c2c(-c3ccc(N(c4ccc(-c5cccc6c5oc5ccccc56)cc4)c4cccc5ccccc45)cc3)ccc3ccccc23)cc1 ; c1ccc(-c2c(-c3ccc(N(c4ccc(-c5cccc6c5oc5ccccc56)cc4)c4ccc5ccccc5c4)cc3)ccc3ccccc23)cc1 ; c1ccc(-c2cccc3ccc(-c4ccc(N(c5ccc(-c6cccc7c6oc6ccccc67)cc5)c5cccc6ccccc56)cc4)cc23)cc1] 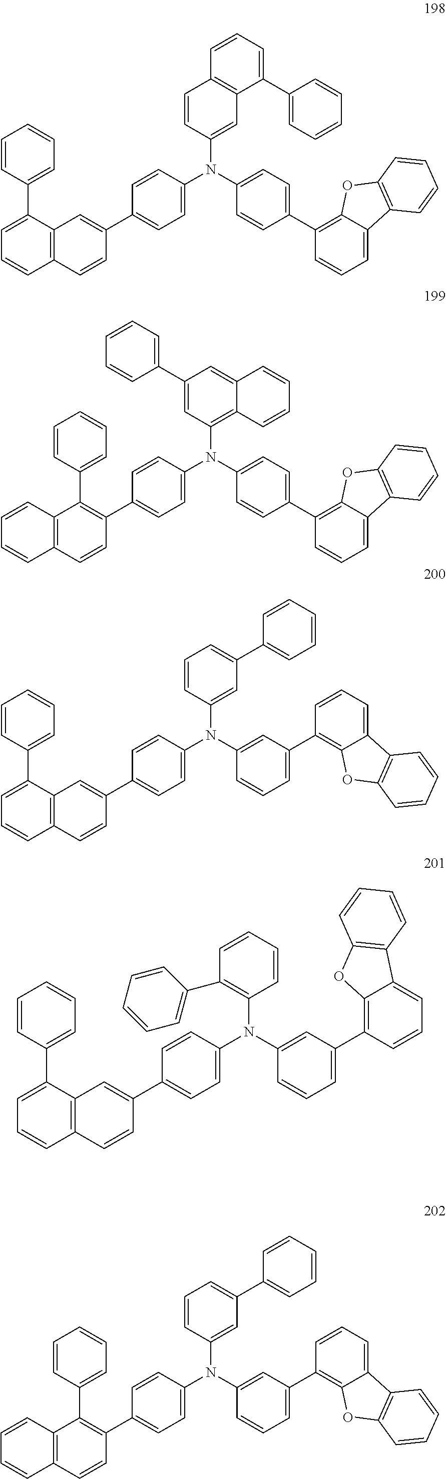 [SMILES: c1ccc(-c2cccc3ccc(-c4ccc(N(c5ccc(-c6cccc7c6oc6ccccc67)cc5)c5ccc6cccc(-c7ccccc7)c6c5)cc4)cc23)cc1 ; c1ccc(-c2cc(N(c3ccc(-c4ccc5ccccc5c4-c4ccccc4)cc3)c3ccc(-c4cccc5c4oc4ccccc45)cc3)c3ccccc3c2)cc1 ; c1ccc(-c2cccc(N(c3ccc(-c4ccc5cccc(-c6ccccc6)c5c4)cc3)c3cccc(-c4cccc5c4oc4ccccc45)c3)c2)cc1 ; c1ccc(-c2ccccc2N(c2ccc(-c3ccc4cccc(-c5ccccc5)c4c3)cc2)c2cccc(-c3cccc4c3oc3ccccc34)c2)cc1 ; c1ccc(-c2cccc(N(c3ccc(-c4ccc5ccccc5c4-c4ccccc4)cc3)c3cccc(-c4cccc5c4oc4ccccc45)c3)c2)cc1] 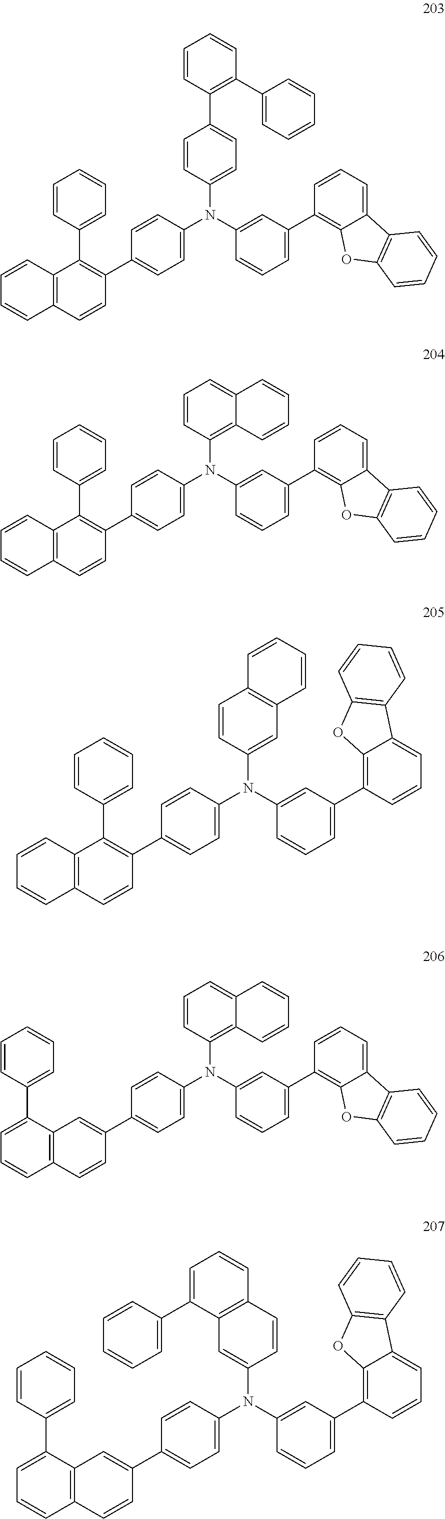 [SMILES: c1ccc(-c2ccccc2-c2ccc(N(c3ccc(-c4ccc5ccccc5c4-c4ccccc4)cc3)c3cccc(-c4cccc5c4oc4ccccc45)c3)cc2)cc1 ; c1ccc(-c2c(-c3ccc(N(c4cccc(-c5cccc6c5oc5ccccc56)c4)c4cccc5ccccc45)cc3)ccc3ccccc23)cc1 ; c1ccc(-c2c(-c3ccc(N(c4cccc(-c5cccc6c5oc5ccccc56)c4)c4ccc5ccccc5c4)cc3)ccc3ccccc23)cc1 ; c1ccc(-c2cccc3ccc(-c4ccc(N(c5cccc(-c6cccc7c6oc6ccccc67)c5)c5cccc6ccccc56)cc4)cc23)cc1 ; c1ccc(-c2cccc3ccc(-c4ccc(N(c5cccc(-c6cccc7c6oc6ccccc67)c5)c5ccc6cccc(-c7ccccc7)c6c5)cc4)cc23)cc1] 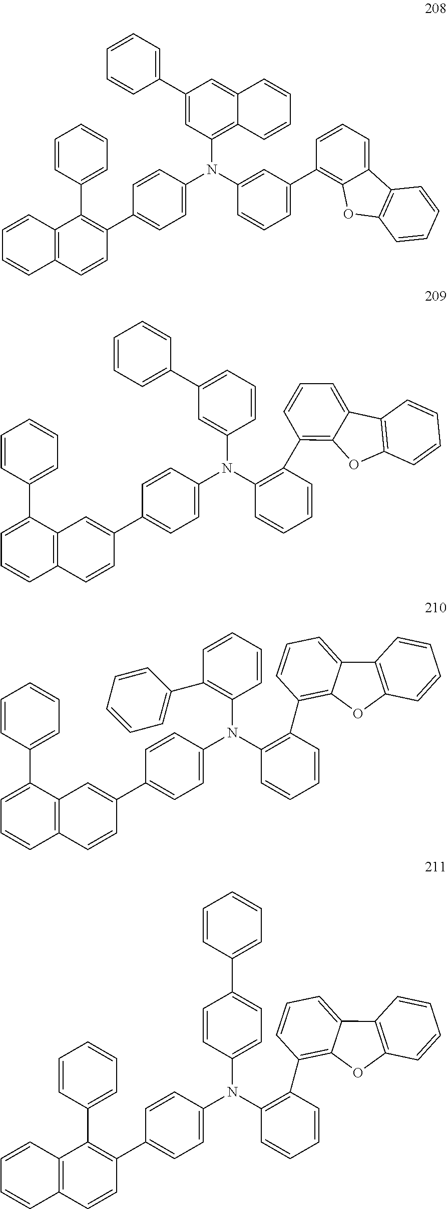 [SMILES: c1ccc(-c2cc(N(c3ccc(-c4ccc5ccccc5c4-c4ccccc4)cc3)c3cccc(-c4cccc5c4oc4ccccc45)c3)c3ccccc3c2)cc1 ; c1ccc(-c2cccc(N(c3ccc(-c4ccc5cccc(-c6ccccc6)c5c4)cc3)c3ccccc3-c3cccc4c3oc3ccccc34)c2)cc1 ; c1ccc(-c2ccccc2N(c2ccc(-c3ccc4cccc(-c5ccccc5)c4c3)cc2)c2ccccc2-c2cccc3c2oc2ccccc23)cc1 ; c1ccc(-c2ccc(N(c3ccc(-c4ccc5ccccc5c4-c4ccccc4)cc3)c3ccccc3-c3cccc4c3oc3ccccc34)cc2)cc1] 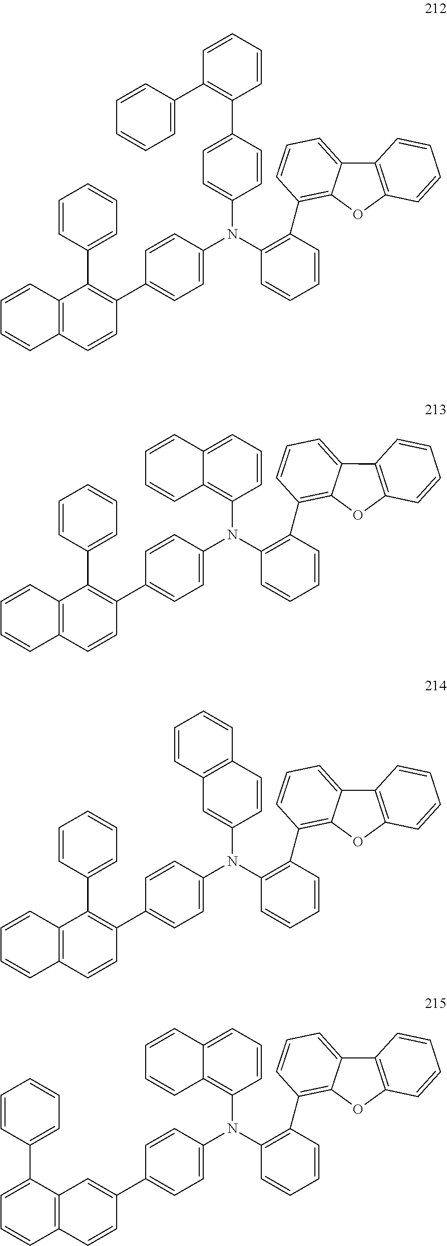 [SMILES: c1ccc(-c2ccccc2-c2ccc(N(c3ccc(-c4ccc5ccccc5c4-c4ccccc4)cc3)c3ccccc3-c3cccc4c3oc3ccccc34)cc2)cc1 ; c1ccc(-c2c(-c3ccc(N(c4ccccc4-c4cccc5c4oc4ccccc45)c4cccc5ccccc45)cc3)ccc3ccccc23)cc1 ; c1ccc(-c2c(-c3ccc(N(c4ccc5ccccc5c4)c4ccccc4-c4cccc5c4oc4ccccc45)cc3)ccc3ccccc23)cc1 ; c1ccc(-c2cccc3ccc(-c4ccc(N(c5ccccc5-c5cccc6c5oc5ccccc56)c5cccc6ccccc56)cc4)cc23)cc1] 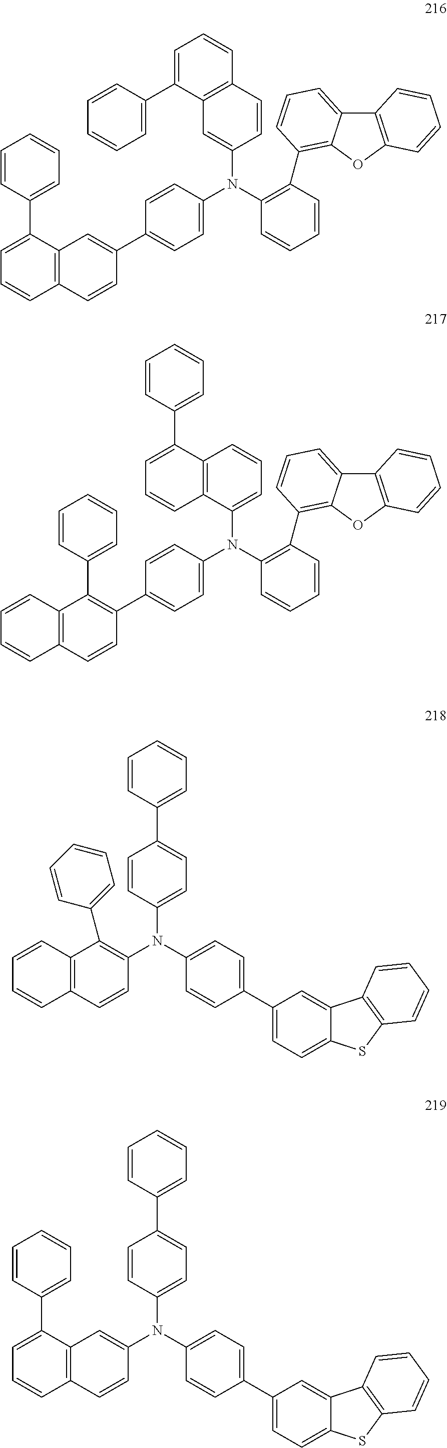 [SMILES: c1ccc(-c2cccc3ccc(-c4ccc(N(c5ccc6cccc(-c7ccccc7)c6c5)c5ccccc5-c5cccc6c5oc5ccccc56)cc4)cc23)cc1 ; c1ccc(-c2c(-c3ccc(N(c4ccccc4-c4cccc5c4oc4ccccc45)c4cccc5c(-c6ccccc6)cccc45)cc3)ccc3ccccc23)cc1 ; c1ccc(-c2ccc(N(c3ccc(-c4ccc5sc6ccccc6c5c4)cc3)c3ccc4ccccc4c3-c3ccccc3)cc2)cc1 ; c1ccc(-c2ccc(N(c3ccc(-c4ccc5sc6ccccc6c5c4)cc3)c3ccc4cccc(-c5ccccc5)c4c3)cc2)cc1] 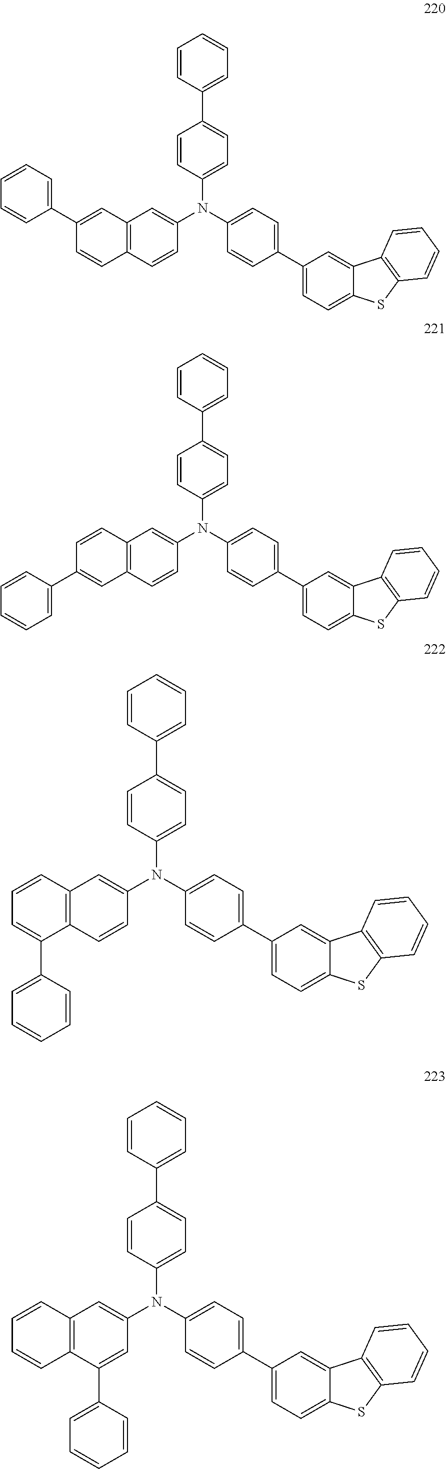 [SMILES: c1ccc(-c2ccc(N(c3ccc(-c4ccc5sc6ccccc6c5c4)cc3)c3ccc4ccc(-c5ccccc5)cc4c3)cc2)cc1 ; c1ccc(-c2ccc(N(c3ccc(-c4ccc5sc6ccccc6c5c4)cc3)c3ccc4cc(-c5ccccc5)ccc4c3)cc2)cc1 ; c1ccc(-c2ccc(N(c3ccc(-c4ccc5sc6ccccc6c5c4)cc3)c3ccc4c(-c5ccccc5)cccc4c3)cc2)cc1 ; c1ccc(-c2ccc(N(c3ccc(-c4ccc5sc6ccccc6c5c4)cc3)c3cc(-c4ccccc4)c4ccccc4c3)cc2)cc1] 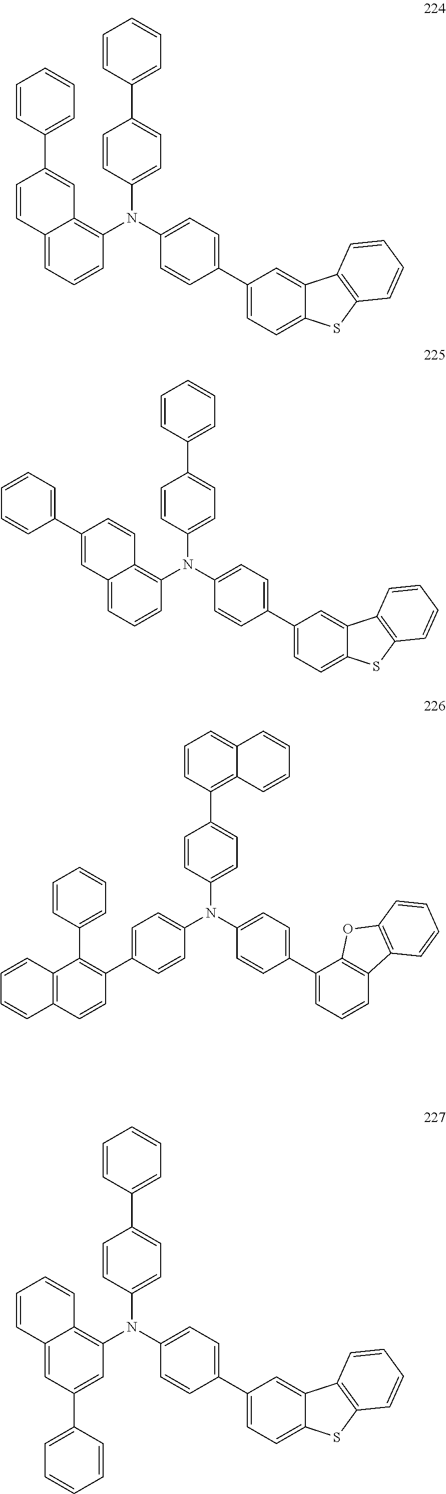 [SMILES: c1ccc(-c2ccc(N(c3ccc(-c4ccc5sc6ccccc6c5c4)cc3)c3cccc4ccc(-c5ccccc5)cc34)cc2)cc1 ; c1ccc(-c2ccc(N(c3ccc(-c4ccc5sc6ccccc6c5c4)cc3)c3cccc4cc(-c5ccccc5)ccc34)cc2)cc1 ; c1ccc(-c2c(-c3ccc(N(c4ccc(-c5cccc6ccccc56)cc4)c4ccc(-c5cccc6c5oc5ccccc56)cc4)cc3)ccc3ccccc23)cc1 ; c1ccc(-c2ccc(N(c3ccc(-c4ccc5sc6ccccc6c5c4)cc3)c3cc(-c4ccccc4)cc4ccccc34)cc2)cc1] 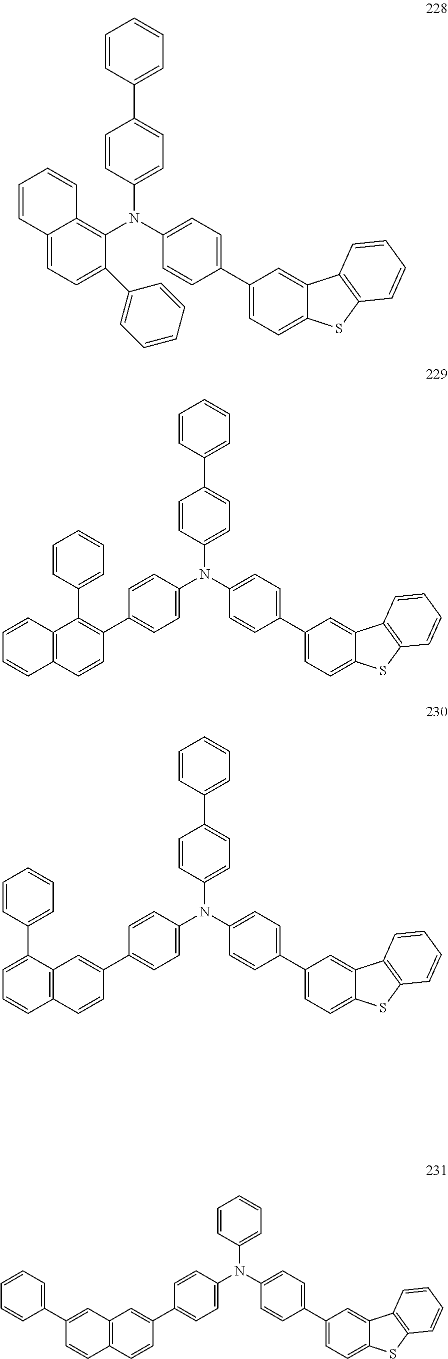 [SMILES: c1ccc(-c2ccc(N(c3ccc(-c4ccc5sc6ccccc6c5c4)cc3)c3c(-c4ccccc4)ccc4ccccc34)cc2)cc1 ; c1ccc(-c2ccc(N(c3ccc(-c4ccc5sc6ccccc6c5c4)cc3)c3ccc(-c4ccc5ccccc5c4-c4ccccc4)cc3)cc2)cc1 ; c1ccc(-c2ccc(N(c3ccc(-c4ccc5cccc(-c6ccccc6)c5c4)cc3)c3ccc(-c4ccc5sc6ccccc6c5c4)cc3)cc2)cc1 ; c1ccc(-c2ccc3ccc(-c4ccc(N(c5ccccc5)c5ccc(-c6ccc7sc8ccccc8c7c6)cc5)cc4)cc3c2)cc1] 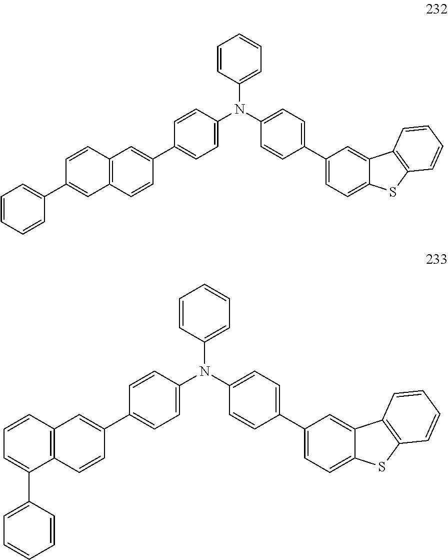 [SMILES: c1ccc(-c2ccc3cc(-c4ccc(N(c5ccccc5)c5ccc(-c6ccc7sc8ccccc8c7c6)cc5)cc4)ccc3c2)cc1 ; c1ccc(-c2cccc3cc(-c4ccc(N(c5ccccc5)c5ccc(-c6ccc7sc8ccccc8c7c6)cc5)cc4)ccc23)cc1] 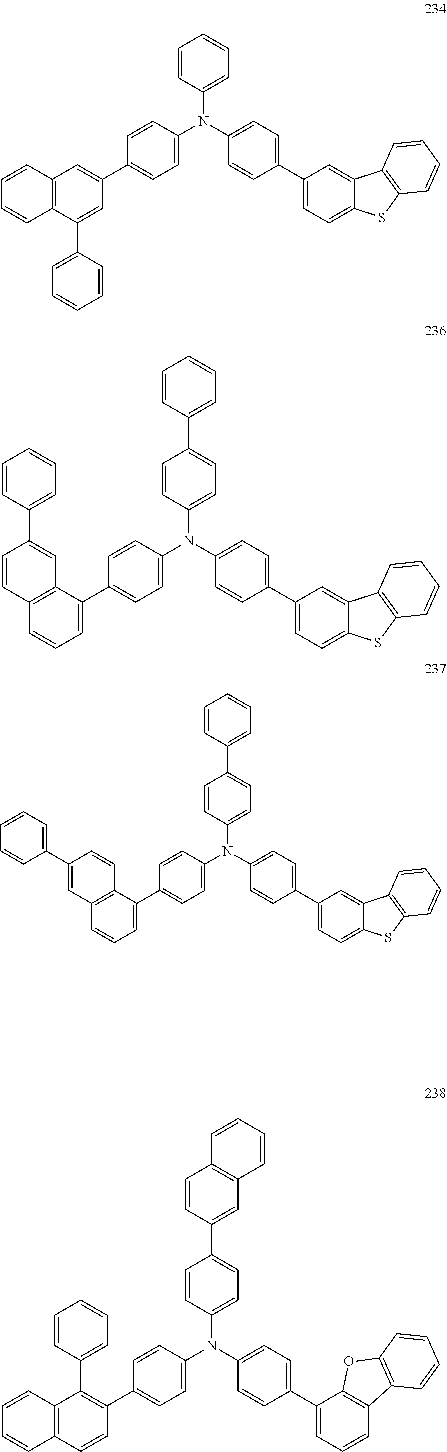 [SMILES: c1ccc(-c2cc(-c3ccc(N(c4ccccc4)c4ccc(-c5ccc6sc7ccccc7c6c5)cc4)cc3)cc3ccccc23)cc1 ; c1ccc(-c2ccc(N(c3ccc(-c4ccc5sc6ccccc6c5c4)cc3)c3ccc(-c4cccc5ccc(-c6ccccc6)cc45)cc3)cc2)cc1 ; c1ccc(-c2ccc(N(c3ccc(-c4ccc5sc6ccccc6c5c4)cc3)c3ccc(-c4cccc5cc(-c6ccccc6)ccc45)cc3)cc2)cc1 ; c1ccc(-c2c(-c3ccc(N(c4ccc(-c5ccc6ccccc6c5)cc4)c4ccc(-c5cccc6c5oc5ccccc56)cc4)cc3)ccc3ccccc23)cc1] 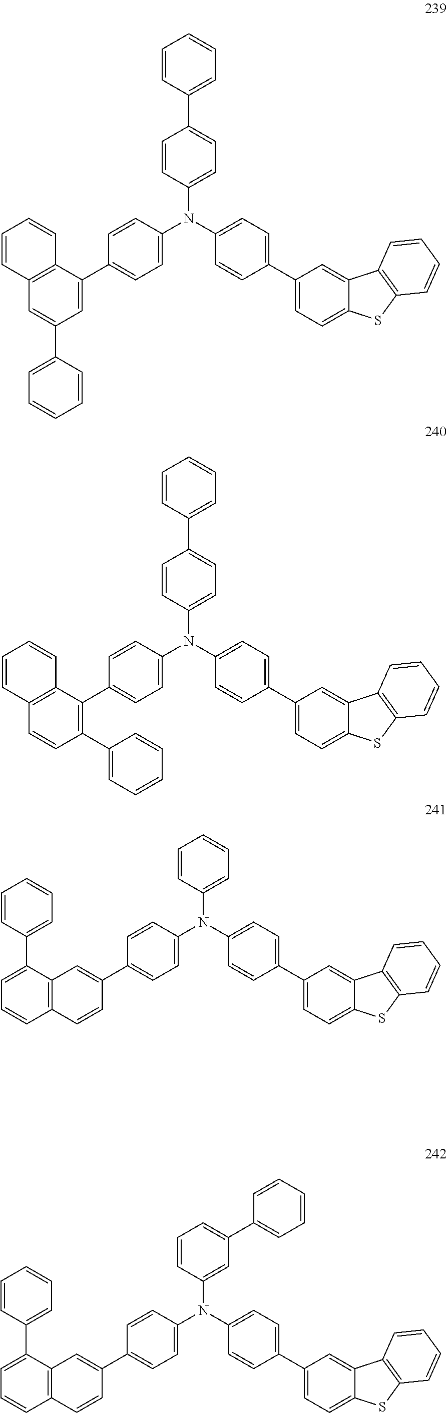 [SMILES: c1ccc(-c2ccc(N(c3ccc(-c4ccc5sc6ccccc6c5c4)cc3)c3ccc(-c4cc(-c5ccccc5)cc5ccccc45)cc3)cc2)cc1 ; c1ccc(-c2ccc(N(c3ccc(-c4ccc5sc6ccccc6c5c4)cc3)c3ccc(-c4c(-c5ccccc5)ccc5ccccc45)cc3)cc2)cc1 ; c1ccc(-c2cccc3ccc(-c4ccc(N(c5ccccc5)c5ccc(-c6ccc7sc8ccccc8c7c6)cc5)cc4)cc23)cc1 ; c1ccc(-c2cccc(N(c3ccc(-c4ccc5cccc(-c6ccccc6)c5c4)cc3)c3ccc(-c4ccc5sc6ccccc6c5c4)cc3)c2)cc1] 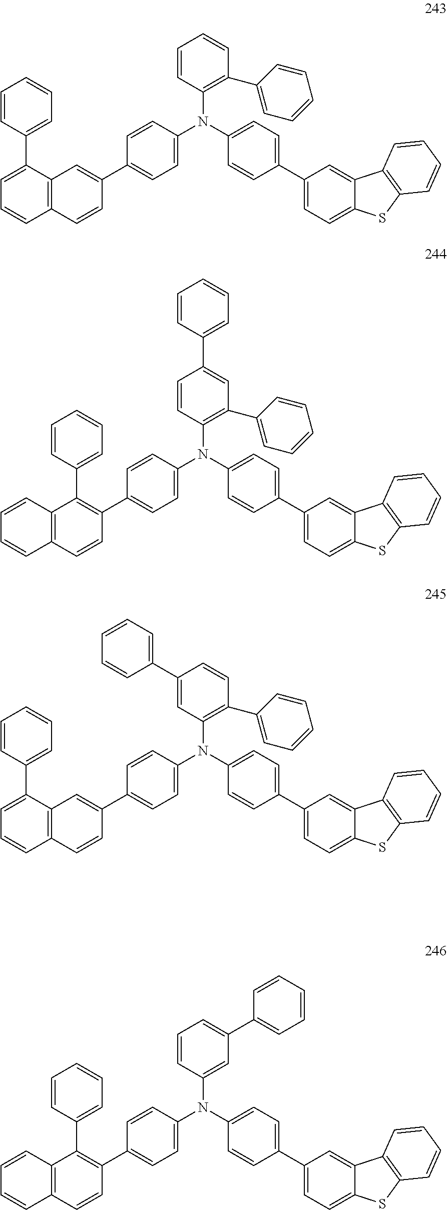 [SMILES: c1ccc(-c2ccccc2N(c2ccc(-c3ccc4cccc(-c5ccccc5)c4c3)cc2)c2ccc(-c3ccc4sc5ccccc5c4c3)cc2)cc1 ; c1ccc(-c2ccc(N(c3ccc(-c4ccc5sc6ccccc6c5c4)cc3)c3ccc(-c4ccc5ccccc5c4-c4ccccc4)cc3)c(-c3ccccc3)c2)cc1 ; c1ccc(-c2ccc(-c3ccccc3)c(N(c3ccc(-c4ccc5cccc(-c6ccccc6)c5c4)cc3)c3ccc(-c4ccc5sc6ccccc6c5c4)cc3)c2)cc1 ; c1ccc(-c2cccc(N(c3ccc(-c4ccc5sc6ccccc6c5c4)cc3)c3ccc(-c4ccc5ccccc5c4-c4ccccc4)cc3)c2)cc1] 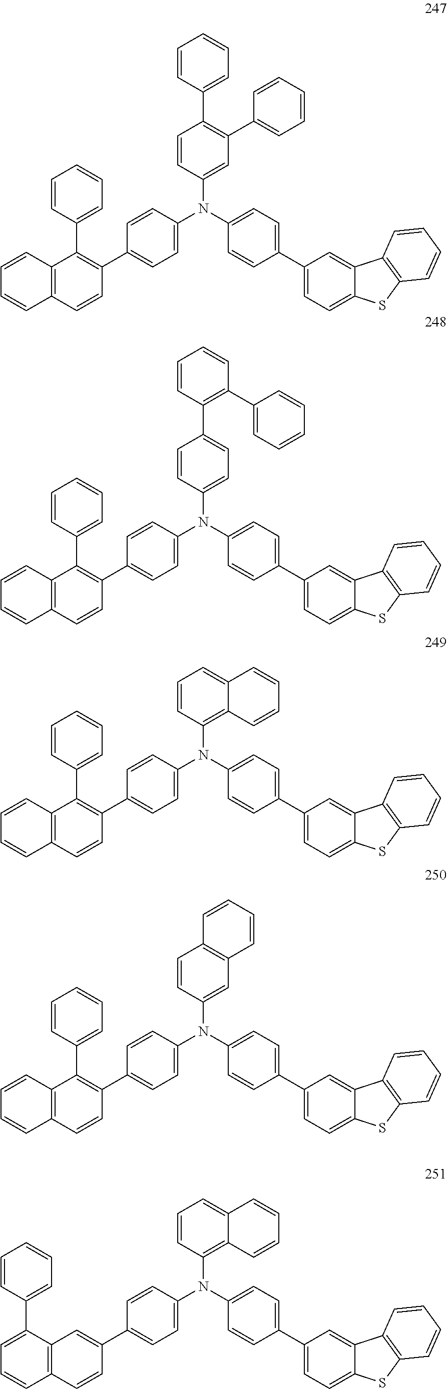 [SMILES: c1ccc(-c2ccc(N(c3ccc(-c4ccc5sc6ccccc6c5c4)cc3)c3ccc(-c4ccc5ccccc5c4-c4ccccc4)cc3)cc2-c2ccccc2)cc1 ; c1ccc(-c2ccccc2-c2ccc(N(c3ccc(-c4ccc5sc6ccccc6c5c4)cc3)c3ccc(-c4ccc5ccccc5c4-c4ccccc4)cc3)cc2)cc1 ; c1ccc(-c2c(-c3ccc(N(c4ccc(-c5ccc6sc7ccccc7c6c5)cc4)c4cccc5ccccc45)cc3)ccc3ccccc23)cc1 ; c1ccc(-c2c(-c3ccc(N(c4ccc(-c5ccc6sc7ccccc7c6c5)cc4)c4ccc5ccccc5c4)cc3)ccc3ccccc23)cc1 ; c1ccc(-c2cccc3ccc(-c4ccc(N(c5ccc(-c6ccc7sc8ccccc8c7c6)cc5)c5cccc6ccccc56)cc4)cc23)cc1] 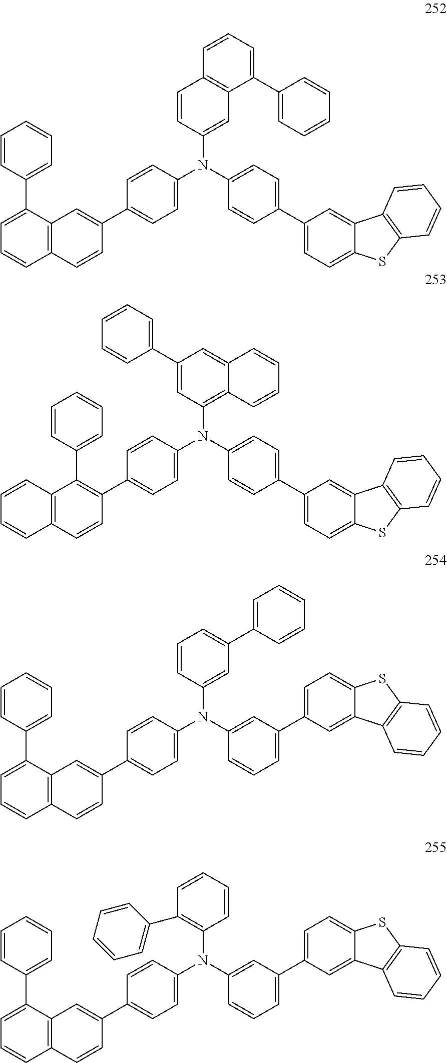 [SMILES: c1ccc(-c2cccc3ccc(-c4ccc(N(c5ccc(-c6ccc7sc8ccccc8c7c6)cc5)c5ccc6cccc(-c7ccccc7)c6c5)cc4)cc23)cc1 ; c1ccc(-c2cc(N(c3ccc(-c4ccc5sc6ccccc6c5c4)cc3)c3ccc(-c4ccc5ccccc5c4-c4ccccc4)cc3)c3ccccc3c2)cc1 ; c1ccc(-c2cccc(N(c3ccc(-c4ccc5cccc(-c6ccccc6)c5c4)cc3)c3cccc(-c4ccc5sc6ccccc6c5c4)c3)c2)cc1 ; c1ccc(-c2ccccc2N(c2ccc(-c3ccc4cccc(-c5ccccc5)c4c3)cc2)c2cccc(-c3ccc4sc5ccccc5c4c3)c2)cc1] 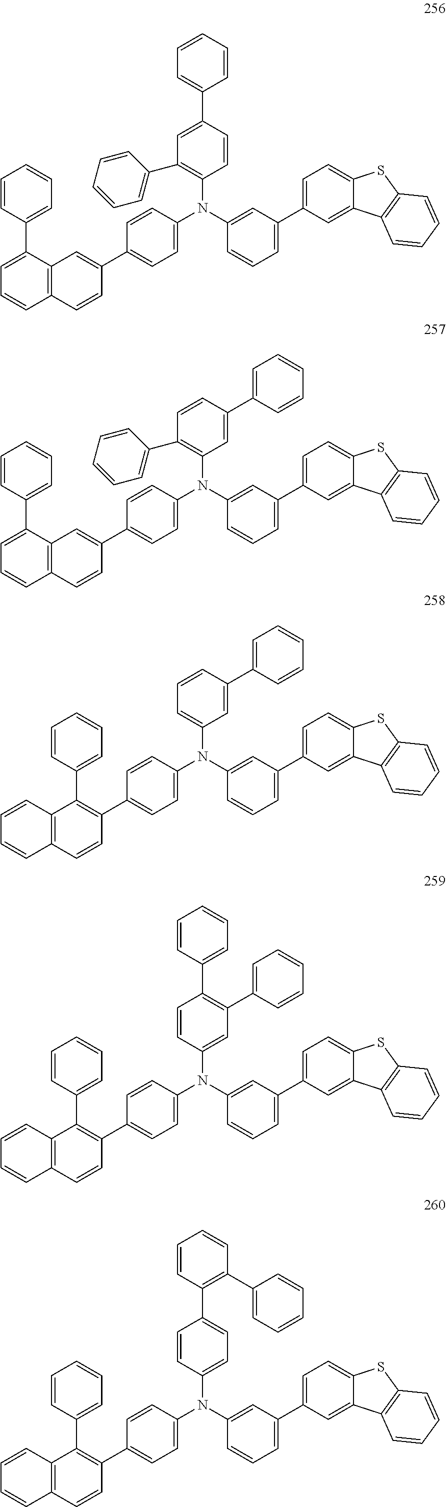 [SMILES: c1ccc(-c2ccc(N(c3ccc(-c4ccc5cccc(-c6ccccc6)c5c4)cc3)c3cccc(-c4ccc5sc6ccccc6c5c4)c3)c(-c3ccccc3)c2)cc1 ; c1ccc(-c2ccc(-c3ccccc3)c(N(c3ccc(-c4ccc5cccc(-c6ccccc6)c5c4)cc3)c3cccc(-c4ccc5sc6ccccc6c5c4)c3)c2)cc1 ; c1ccc(-c2cccc(N(c3ccc(-c4ccc5ccccc5c4-c4ccccc4)cc3)c3cccc(-c4ccc5sc6ccccc6c5c4)c3)c2)cc1 ; c1ccc(-c2ccc(N(c3ccc(-c4ccc5ccccc5c4-c4ccccc4)cc3)c3cccc(-c4ccc5sc6ccccc6c5c4)c3)cc2-c2ccccc2)cc1 ; c1ccc(-c2ccccc2-c2ccc(N(c3ccc(-c4ccc5ccccc5c4-c4ccccc4)cc3)c3cccc(-c4ccc5sc6ccccc6c5c4)c3)cc2)cc1] 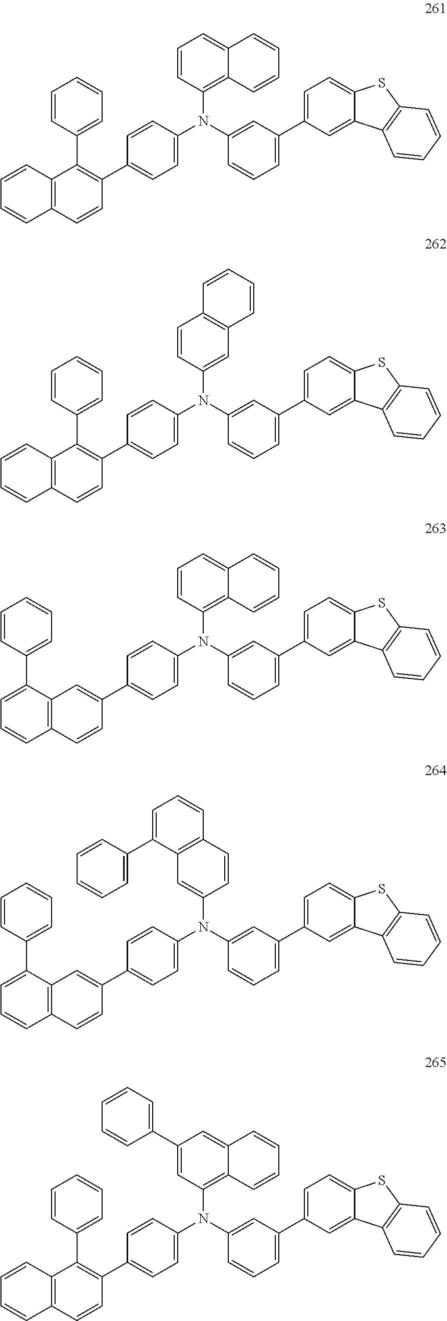 [SMILES: c1ccc(-c2c(-c3ccc(N(c4cccc(-c5ccc6sc7ccccc7c6c5)c4)c4cccc5ccccc45)cc3)ccc3ccccc23)cc1 ; c1ccc(-c2c(-c3ccc(N(c4cccc(-c5ccc6sc7ccccc7c6c5)c4)c4ccc5ccccc5c4)cc3)ccc3ccccc23)cc1 ; c1ccc(-c2cccc3ccc(-c4ccc(N(c5cccc(-c6ccc7sc8ccccc8c7c6)c5)c5cccc6ccccc56)cc4)cc23)cc1 ; c1ccc(-c2cccc3ccc(-c4ccc(N(c5cccc(-c6ccc7sc8ccccc8c7c6)c5)c5ccc6cccc(-c7ccccc7)c6c5)cc4)cc23)cc1 ; c1ccc(-c2cc(N(c3ccc(-c4ccc5ccccc5c4-c4ccccc4)cc3)c3cccc(-c4ccc5sc6ccccc6c5c4)c3)c3ccccc3c2)cc1] 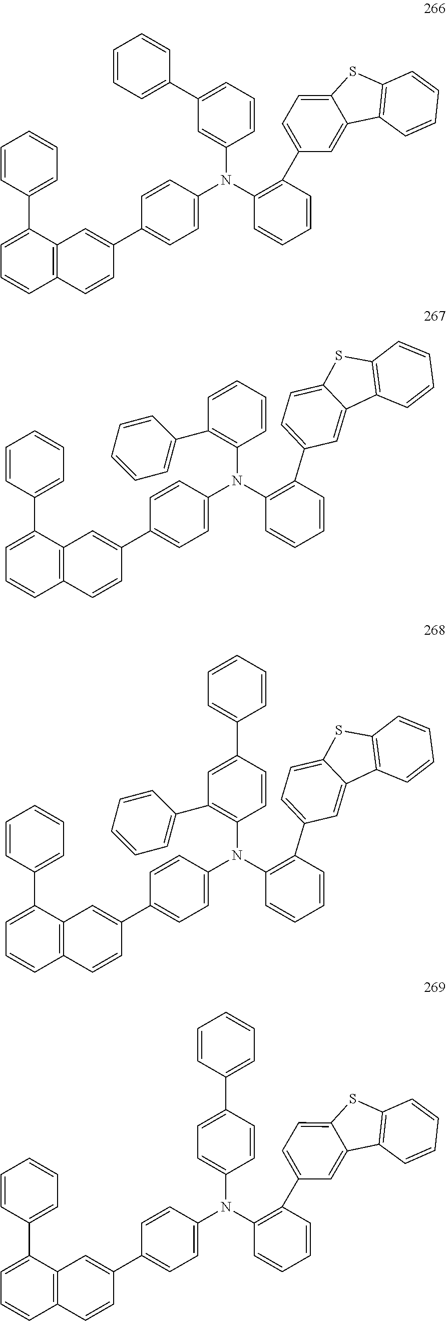 [SMILES: c1ccc(-c2cccc(N(c3ccc(-c4ccc5cccc(-c6ccccc6)c5c4)cc3)c3ccccc3-c3ccc4sc5ccccc5c4c3)c2)cc1 ; c1ccc(-c2ccccc2N(c2ccc(-c3ccc4cccc(-c5ccccc5)c4c3)cc2)c2ccccc2-c2ccc3sc4ccccc4c3c2)cc1 ; c1ccc(-c2ccc(N(c3ccc(-c4ccc5cccc(-c6ccccc6)c5c4)cc3)c3ccccc3-c3ccc4sc5ccccc5c4c3)c(-c3ccccc3)c2)cc1 ; c1ccc(-c2ccc(N(c3ccc(-c4ccc5cccc(-c6ccccc6)c5c4)cc3)c3ccccc3-c3ccc4sc5ccccc5c4c3)cc2)cc1] 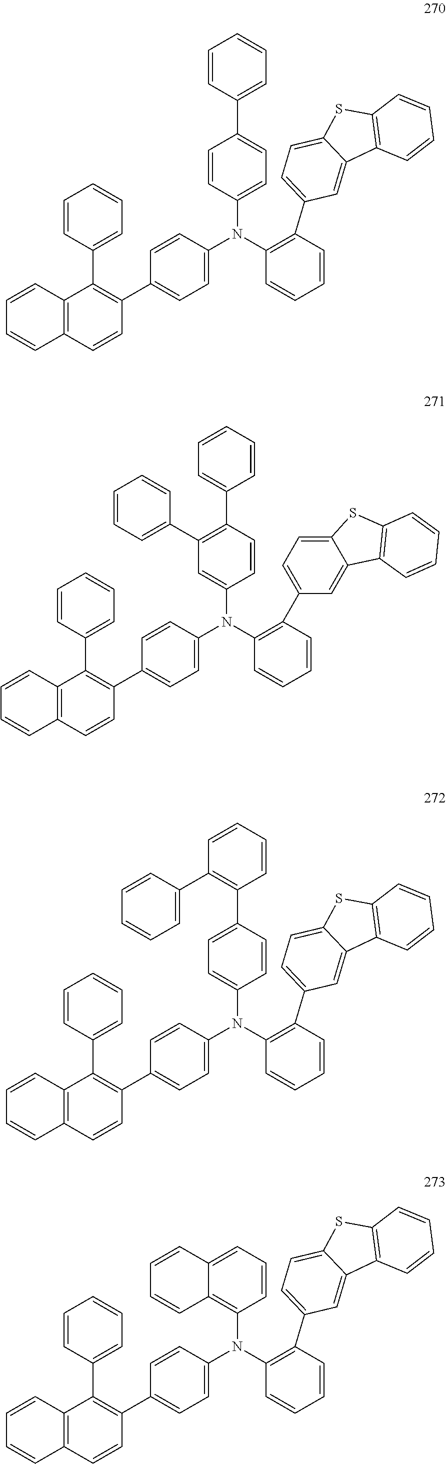 [SMILES: c1ccc(-c2ccc(N(c3ccc(-c4ccc5ccccc5c4-c4ccccc4)cc3)c3ccccc3-c3ccc4sc5ccccc5c4c3)cc2)cc1 ; c1ccc(-c2ccc(N(c3ccc(-c4ccc5ccccc5c4-c4ccccc4)cc3)c3ccccc3-c3ccc4sc5ccccc5c4c3)cc2-c2ccccc2)cc1 ; c1ccc(-c2ccccc2-c2ccc(N(c3ccc(-c4ccc5ccccc5c4-c4ccccc4)cc3)c3ccccc3-c3ccc4sc5ccccc5c4c3)cc2)cc1 ; c1ccc(-c2c(-c3ccc(N(c4ccccc4-c4ccc5sc6ccccc6c5c4)c4cccc5ccccc45)cc3)ccc3ccccc23)cc1] 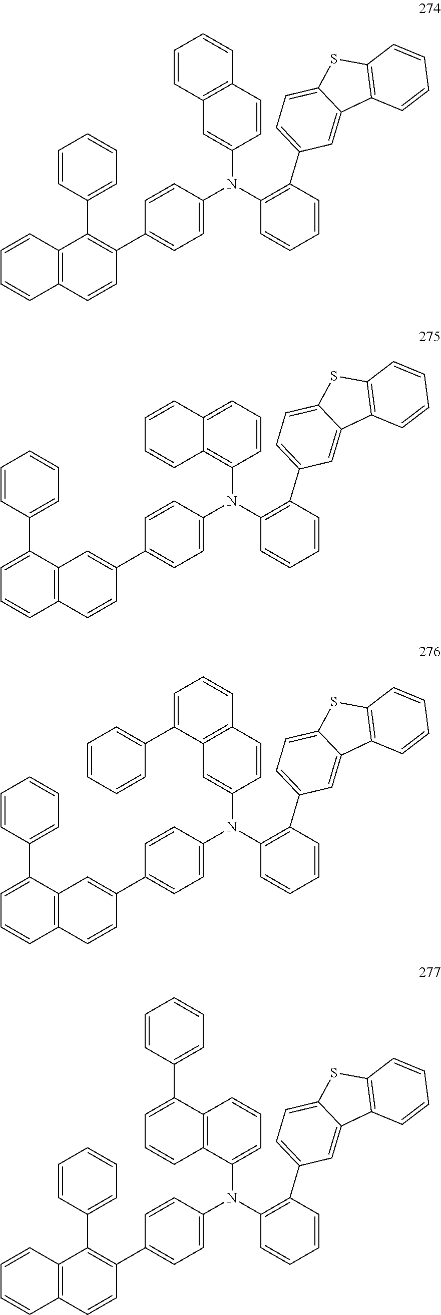 [SMILES: c1ccc(-c2c(-c3ccc(N(c4ccc5ccccc5c4)c4ccccc4-c4ccc5sc6ccccc6c5c4)cc3)ccc3ccccc23)cc1 ; c1ccc(-c2cccc3ccc(-c4ccc(N(c5ccccc5-c5ccc6sc7ccccc7c6c5)c5cccc6ccccc56)cc4)cc23)cc1 ; c1ccc(-c2cccc3ccc(-c4ccc(N(c5ccc6cccc(-c7ccccc7)c6c5)c5ccccc5-c5ccc6sc7ccccc7c6c5)cc4)cc23)cc1 ; c1ccc(-c2c(-c3ccc(N(c4ccccc4-c4ccc5sc6ccccc6c5c4)c4cccc5c(-c6ccccc6)cccc45)cc3)ccc3ccccc23)cc1] 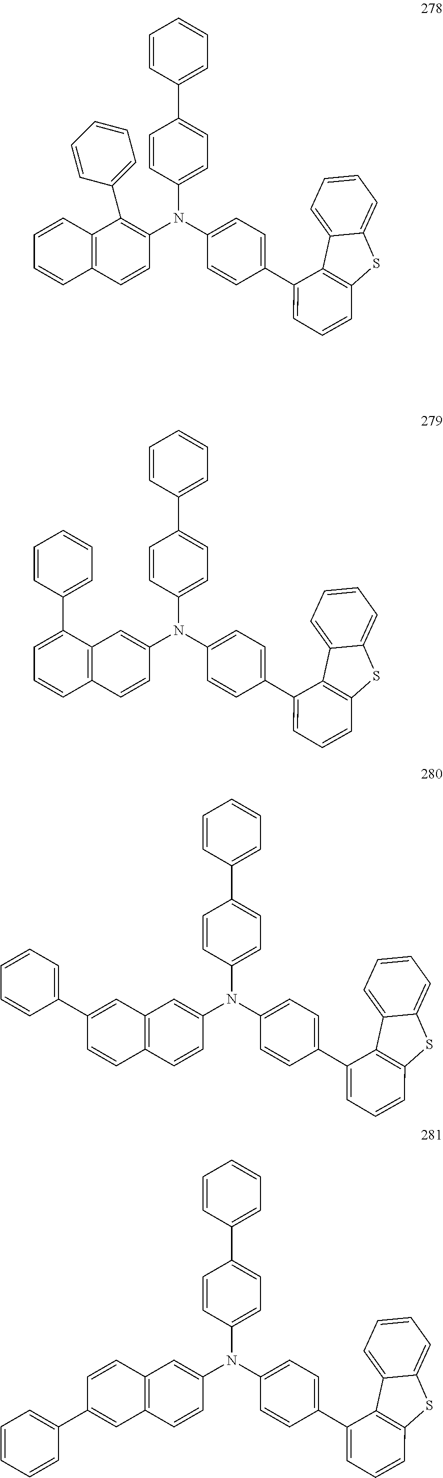 [SMILES: c1ccc(-c2ccc(N(c3ccc(-c4cccc5sc6ccccc6c45)cc3)c3ccc4ccccc4c3-c3ccccc3)cc2)cc1 ; c1ccc(-c2ccc(N(c3ccc(-c4cccc5sc6ccccc6c45)cc3)c3ccc4cccc(-c5ccccc5)c4c3)cc2)cc1 ; c1ccc(-c2ccc(N(c3ccc(-c4cccc5sc6ccccc6c45)cc3)c3ccc4ccc(-c5ccccc5)cc4c3)cc2)cc1 ; c1ccc(-c2ccc(N(c3ccc(-c4cccc5sc6ccccc6c45)cc3)c3ccc4cc(-c5ccccc5)ccc4c3)cc2)cc1] 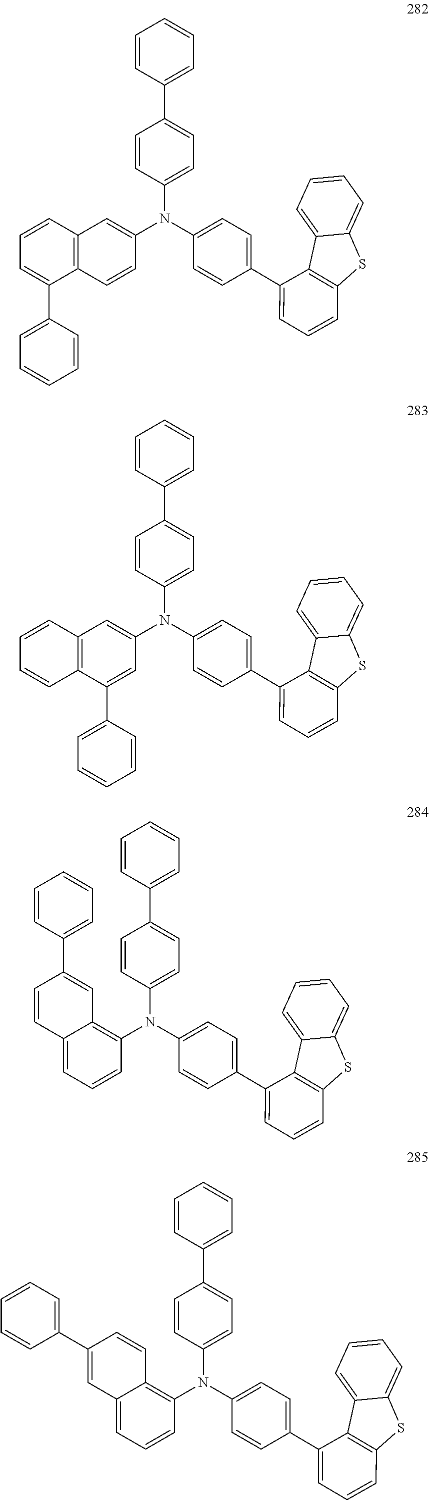 [SMILES: c1ccc(-c2ccc(N(c3ccc(-c4cccc5sc6ccccc6c45)cc3)c3ccc4c(-c5ccccc5)cccc4c3)cc2)cc1 ; c1ccc(-c2ccc(N(c3ccc(-c4cccc5sc6ccccc6c45)cc3)c3cc(-c4ccccc4)c4ccccc4c3)cc2)cc1 ; c1ccc(-c2ccc(N(c3ccc(-c4cccc5sc6ccccc6c45)cc3)c3cccc4ccc(-c5ccccc5)cc34)cc2)cc1 ; c1ccc(-c2ccc(N(c3ccc(-c4cccc5sc6ccccc6c45)cc3)c3cccc4cc(-c5ccccc5)ccc34)cc2)cc1] 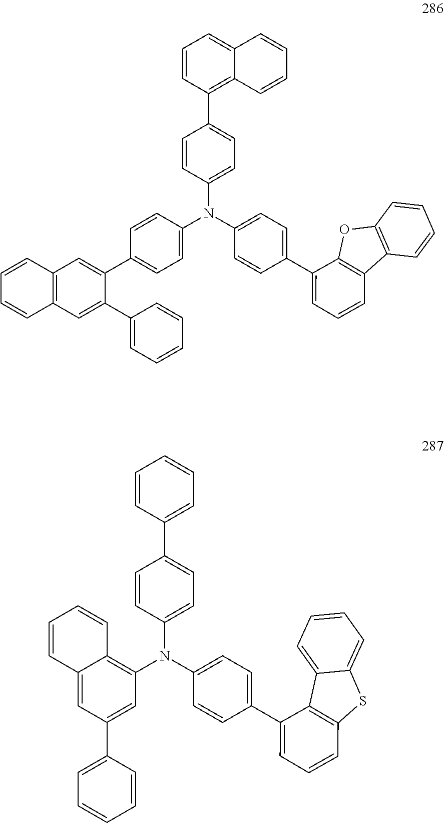 [SMILES: c1ccc(-c2cc3ccccc3cc2-c2ccc(N(c3ccc(-c4cccc5ccccc45)cc3)c3ccc(-c4cccc5c4oc4ccccc45)cc3)cc2)cc1 ; c1ccc(-c2ccc(N(c3ccc(-c4cccc5sc6ccccc6c45)cc3)c3cc(-c4ccccc4)cc4ccccc34)cc2)cc1] 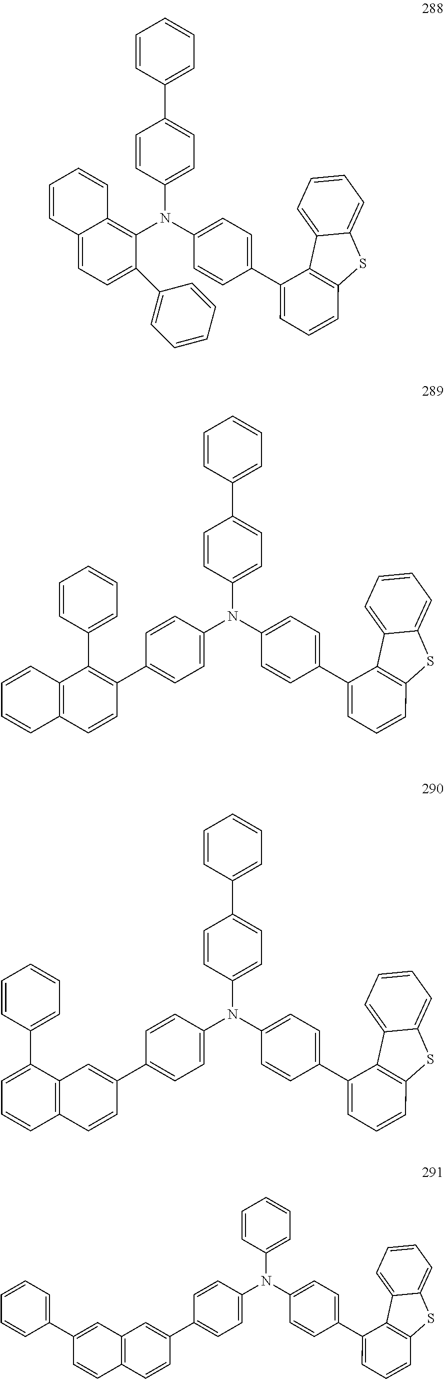 [SMILES: c1ccc(-c2ccc(N(c3ccc(-c4cccc5sc6ccccc6c45)cc3)c3c(-c4ccccc4)ccc4ccccc34)cc2)cc1 ; c1ccc(-c2ccc(N(c3ccc(-c4ccc5ccccc5c4-c4ccccc4)cc3)c3ccc(-c4cccc5sc6ccccc6c45)cc3)cc2)cc1 ; c1ccc(-c2ccc(N(c3ccc(-c4ccc5cccc(-c6ccccc6)c5c4)cc3)c3ccc(-c4cccc5sc6ccccc6c45)cc3)cc2)cc1 ; c1ccc(-c2ccc3ccc(-c4ccc(N(c5ccccc5)c5ccc(-c6cccc7sc8ccccc8c67)cc5)cc4)cc3c2)cc1] 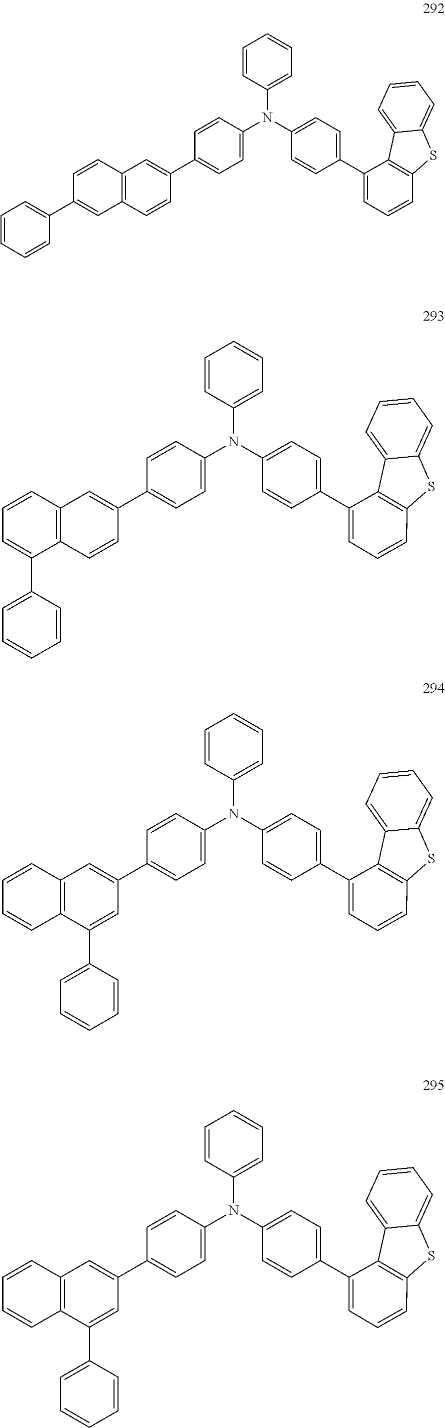 [SMILES: c1ccc(-c2ccc3cc(-c4ccc(N(c5ccccc5)c5ccc(-c6cccc7sc8ccccc8c67)cc5)cc4)ccc3c2)cc1 ; c1ccc(-c2cccc3cc(-c4ccc(N(c5ccccc5)c5ccc(-c6cccc7sc8ccccc8c67)cc5)cc4)ccc23)cc1 ; c1ccc(-c2cc(-c3ccc(N(c4ccccc4)c4ccc(-c5cccc6sc7ccccc7c56)cc4)cc3)cc3ccccc23)cc1 ; c1ccc(-c2cc(-c3ccc(N(c4ccccc4)c4ccc(-c5cccc6sc7ccccc7c56)cc4)cc3)cc3ccccc23)cc1] 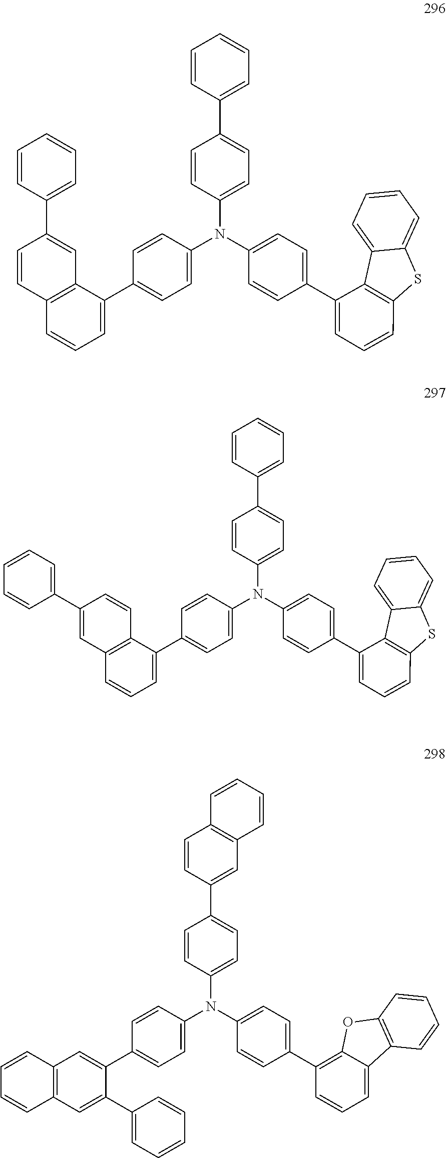 [SMILES: c1ccc(-c2ccc(N(c3ccc(-c4cccc5ccc(-c6ccccc6)cc45)cc3)c3ccc(-c4cccc5sc6ccccc6c45)cc3)cc2)cc1 ; c1ccc(-c2ccc(N(c3ccc(-c4cccc5cc(-c6ccccc6)ccc45)cc3)c3ccc(-c4cccc5sc6ccccc6c45)cc3)cc2)cc1 ; c1ccc(-c2cc3ccccc3cc2-c2ccc(N(c3ccc(-c4ccc5ccccc5c4)cc3)c3ccc(-c4cccc5c4oc4ccccc45)cc3)cc2)cc1] 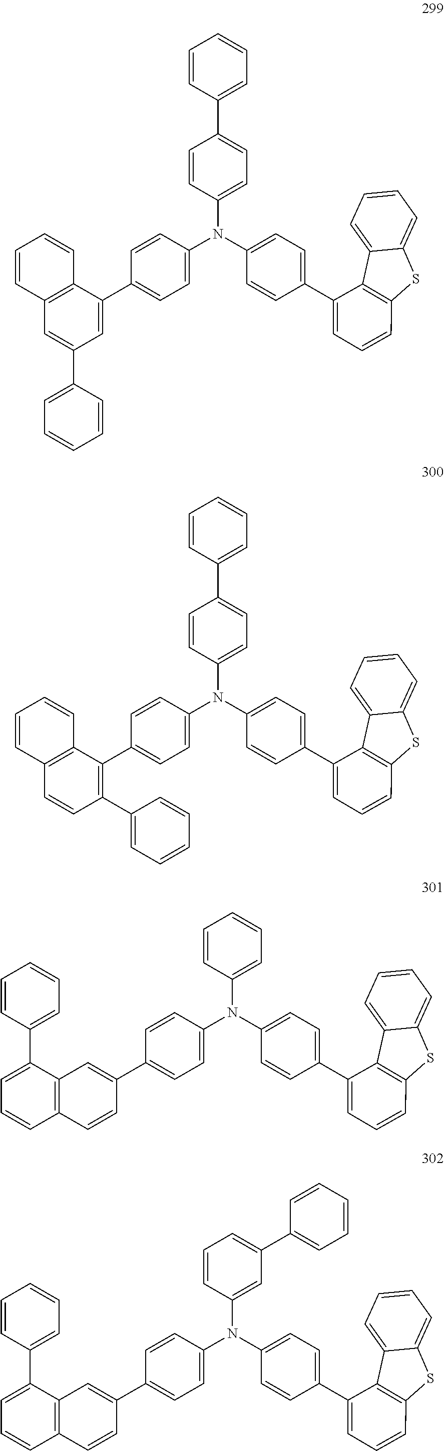 [SMILES: c1ccc(-c2ccc(N(c3ccc(-c4cc(-c5ccccc5)cc5ccccc45)cc3)c3ccc(-c4cccc5sc6ccccc6c45)cc3)cc2)cc1 ; c1ccc(-c2ccc(N(c3ccc(-c4c(-c5ccccc5)ccc5ccccc45)cc3)c3ccc(-c4cccc5sc6ccccc6c45)cc3)cc2)cc1 ; c1ccc(-c2cccc3ccc(-c4ccc(N(c5ccccc5)c5ccc(-c6cccc7sc8ccccc8c67)cc5)cc4)cc23)cc1 ; c1ccc(-c2cccc(N(c3ccc(-c4ccc5cccc(-c6ccccc6)c5c4)cc3)c3ccc(-c4cccc5sc6ccccc6c45)cc3)c2)cc1] 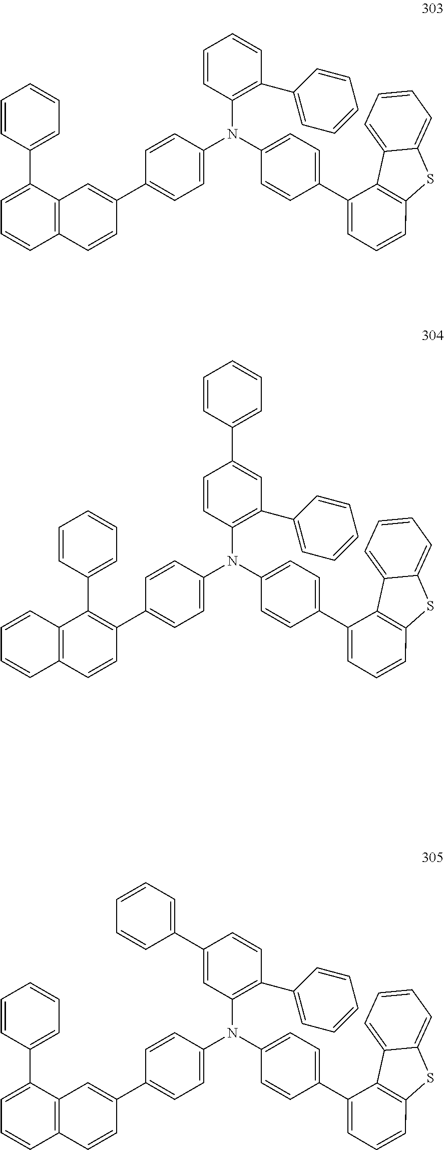 [SMILES: c1ccc(-c2ccccc2N(c2ccc(-c3ccc4cccc(-c5ccccc5)c4c3)cc2)c2ccc(-c3cccc4sc5ccccc5c34)cc2)cc1 ; c1ccc(-c2ccc(N(c3ccc(-c4ccc5ccccc5c4-c4ccccc4)cc3)c3ccc(-c4cccc5sc6ccccc6c45)cc3)c(-c3ccccc3)c2)cc1 ; c1ccc(-c2ccc(-c3ccccc3)c(N(c3ccc(-c4ccc5cccc(-c6ccccc6)c5c4)cc3)c3ccc(-c4cccc5sc6ccccc6c45)cc3)c2)cc1] 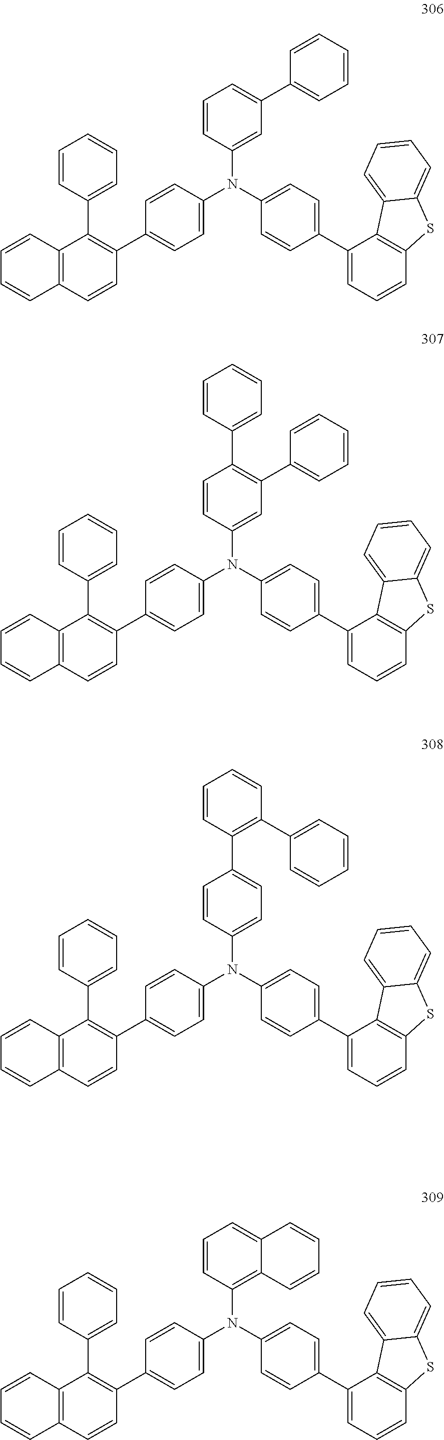 [SMILES: c1ccc(-c2cccc(N(c3ccc(-c4ccc5ccccc5c4-c4ccccc4)cc3)c3ccc(-c4cccc5sc6ccccc6c45)cc3)c2)cc1 ; c1ccc(-c2ccc(N(c3ccc(-c4ccc5ccccc5c4-c4ccccc4)cc3)c3ccc(-c4cccc5sc6ccccc6c45)cc3)cc2-c2ccccc2)cc1 ; c1ccc(-c2ccccc2-c2ccc(N(c3ccc(-c4ccc5ccccc5c4-c4ccccc4)cc3)c3ccc(-c4cccc5sc6ccccc6c45)cc3)cc2)cc1 ; c1ccc(-c2c(-c3ccc(N(c4ccc(-c5cccc6sc7ccccc7c56)cc4)c4cccc5ccccc45)cc3)ccc3ccccc23)cc1] 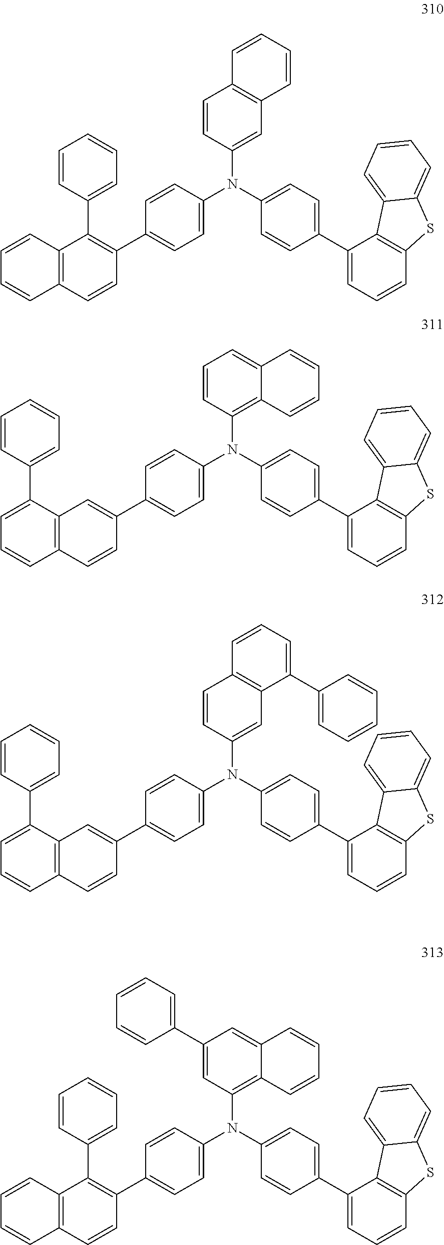 [SMILES: c1ccc(-c2c(-c3ccc(N(c4ccc(-c5cccc6sc7ccccc7c56)cc4)c4ccc5ccccc5c4)cc3)ccc3ccccc23)cc1 ; c1ccc(-c2cccc3ccc(-c4ccc(N(c5ccc(-c6cccc7sc8ccccc8c67)cc5)c5cccc6ccccc56)cc4)cc23)cc1 ; c1ccc(-c2cccc3ccc(-c4ccc(N(c5ccc(-c6cccc7sc8ccccc8c67)cc5)c5ccc6cccc(-c7ccccc7)c6c5)cc4)cc23)cc1 ; c1ccc(-c2cc(N(c3ccc(-c4ccc5ccccc5c4-c4ccccc4)cc3)c3ccc(-c4cccc5sc6ccccc6c45)cc3)c3ccccc3c2)cc1] 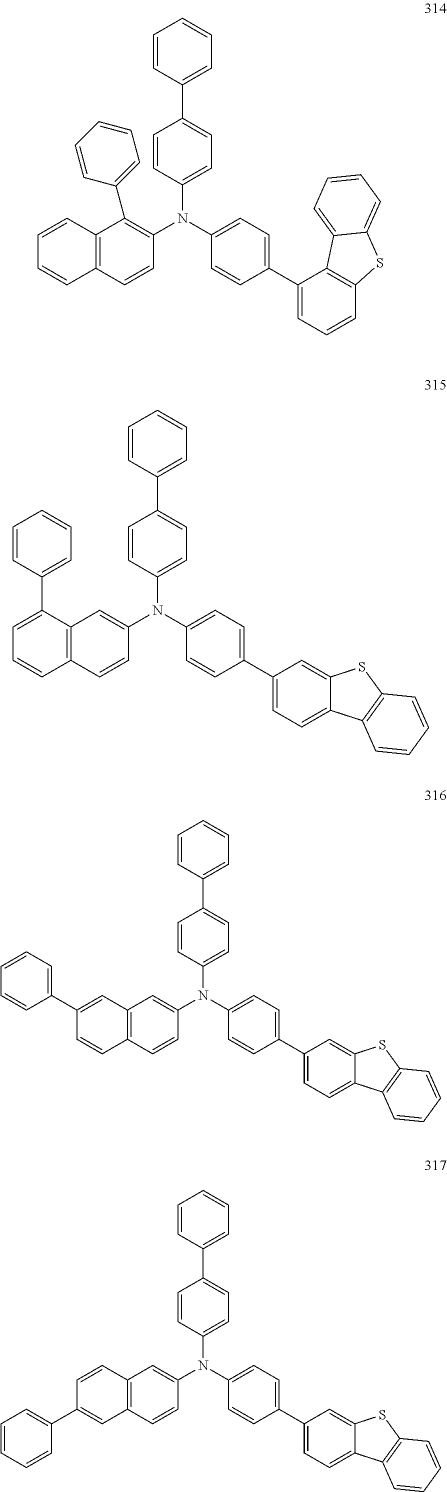 [SMILES: c1ccc(-c2ccc(N(c3ccc(-c4cccc5sc6ccccc6c45)cc3)c3ccc4ccccc4c3-c3ccccc3)cc2)cc1 ; c1ccc(-c2ccc(N(c3ccc(-c4ccc5c(c4)sc4ccccc45)cc3)c3ccc4cccc(-c5ccccc5)c4c3)cc2)cc1 ; c1ccc(-c2ccc(N(c3ccc(-c4ccc5c(c4)sc4ccccc45)cc3)c3ccc4ccc(-c5ccccc5)cc4c3)cc2)cc1 ; c1ccc(-c2ccc(N(c3ccc(-c4ccc5c(c4)sc4ccccc45)cc3)c3ccc4cc(-c5ccccc5)ccc4c3)cc2)cc1] 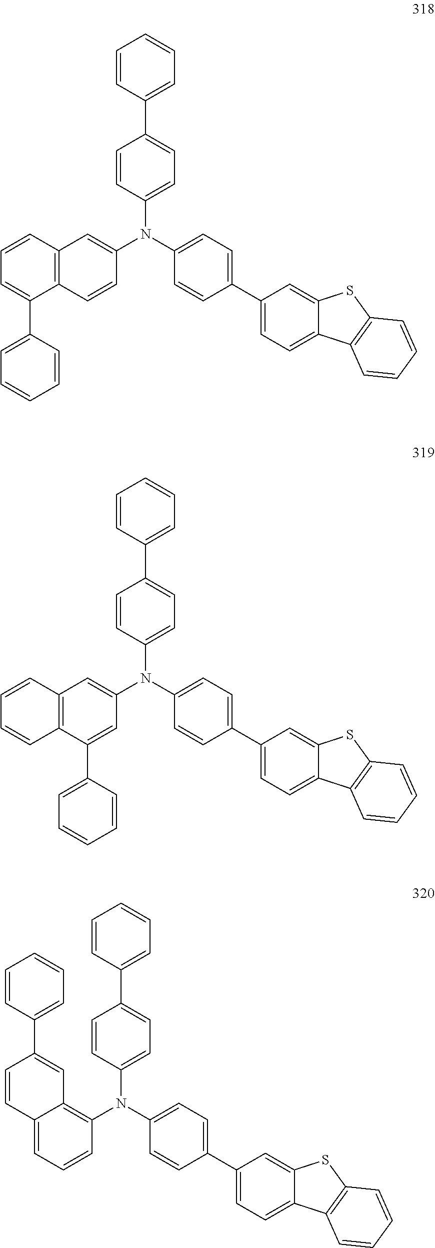 [SMILES: c1ccc(-c2ccc(N(c3ccc(-c4ccc5c(c4)sc4ccccc45)cc3)c3ccc4c(-c5ccccc5)cccc4c3)cc2)cc1 ; c1ccc(-c2ccc(N(c3ccc(-c4ccc5c(c4)sc4ccccc45)cc3)c3cc(-c4ccccc4)c4ccccc4c3)cc2)cc1 ; c1ccc(-c2ccc(N(c3ccc(-c4ccc5c(c4)sc4ccccc45)cc3)c3cccc4ccc(-c5ccccc5)cc34)cc2)cc1] 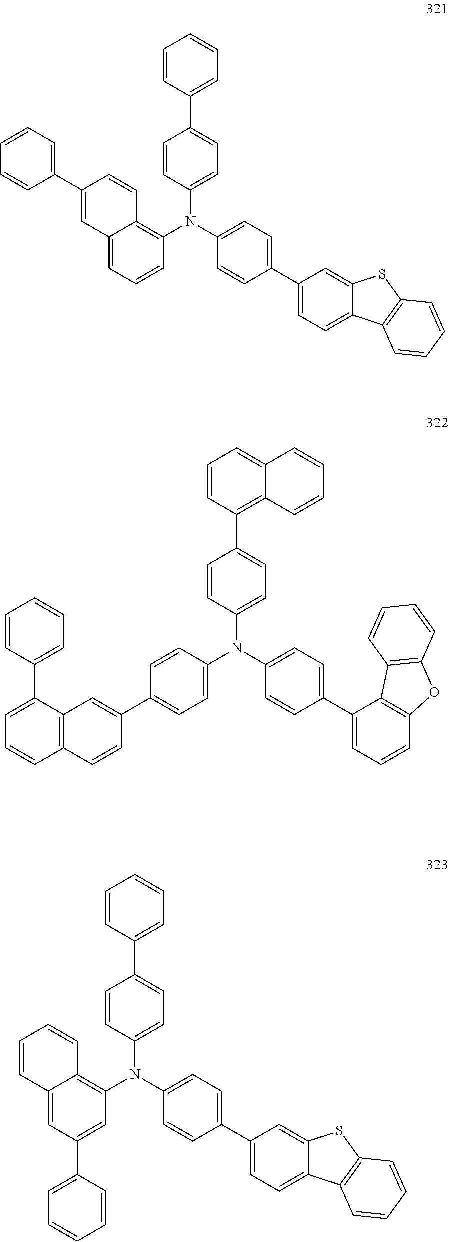 [SMILES: c1ccc(-c2ccc(N(c3ccc(-c4ccc5c(c4)sc4ccccc45)cc3)c3cccc4cc(-c5ccccc5)ccc34)cc2)cc1 ; c1ccc(-c2cccc3ccc(-c4ccc(N(c5ccc(-c6cccc7ccccc67)cc5)c5ccc(-c6cccc7oc8ccccc8c67)cc5)cc4)cc23)cc1 ; c1ccc(-c2ccc(N(c3ccc(-c4ccc5c(c4)sc4ccccc45)cc3)c3cc(-c4ccccc4)cc4ccccc34)cc2)cc1] 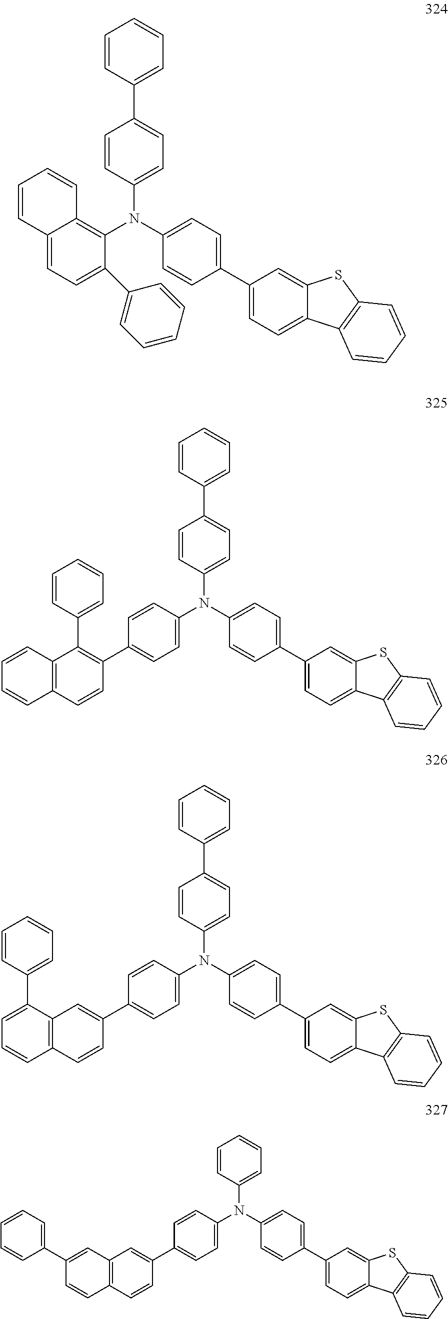 [SMILES: c1ccc(-c2ccc(N(c3ccc(-c4ccc5c(c4)sc4ccccc45)cc3)c3c(-c4ccccc4)ccc4ccccc34)cc2)cc1 ; c1ccc(-c2ccc(N(c3ccc(-c4ccc5c(c4)sc4ccccc45)cc3)c3ccc(-c4ccc5ccccc5c4-c4ccccc4)cc3)cc2)cc1 ; c1ccc(-c2ccc(N(c3ccc(-c4ccc5c(c4)sc4ccccc45)cc3)c3ccc(-c4ccc5cccc(-c6ccccc6)c5c4)cc3)cc2)cc1 ; c1ccc(-c2ccc3ccc(-c4ccc(N(c5ccccc5)c5ccc(-c6ccc7c(c6)sc6ccccc67)cc5)cc4)cc3c2)cc1] 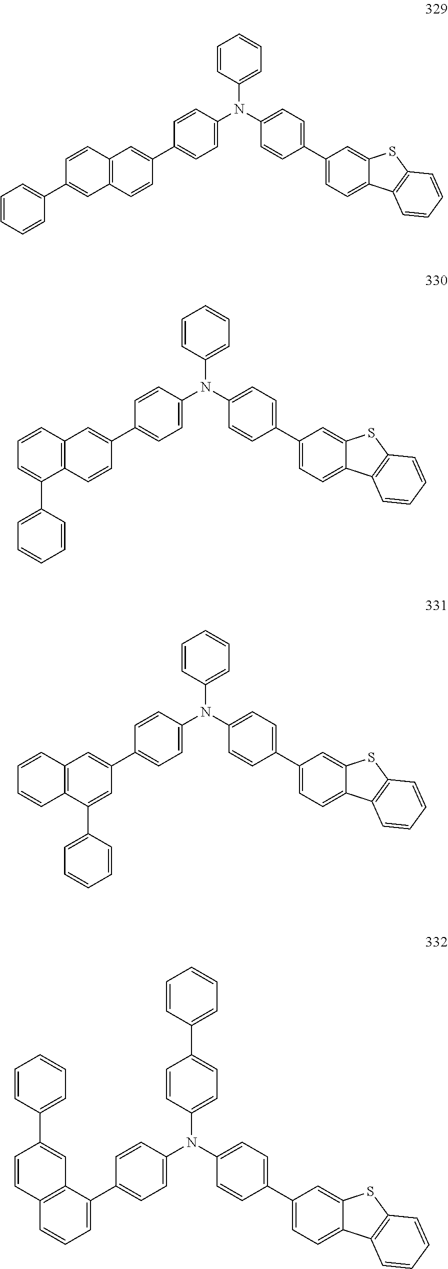 [SMILES: c1ccc(-c2ccc3cc(-c4ccc(N(c5ccccc5)c5ccc(-c6ccc7c(c6)sc6ccccc67)cc5)cc4)ccc3c2)cc1 ; c1ccc(-c2cccc3cc(-c4ccc(N(c5ccccc5)c5ccc(-c6ccc7c(c6)sc6ccccc67)cc5)cc4)ccc23)cc1 ; c1ccc(-c2cc(-c3ccc(N(c4ccccc4)c4ccc(-c5ccc6c(c5)sc5ccccc56)cc4)cc3)cc3ccccc23)cc1 ; c1ccc(-c2ccc(N(c3ccc(-c4ccc5c(c4)sc4ccccc45)cc3)c3ccc(-c4cccc5ccc(-c6ccccc6)cc45)cc3)cc2)cc1] 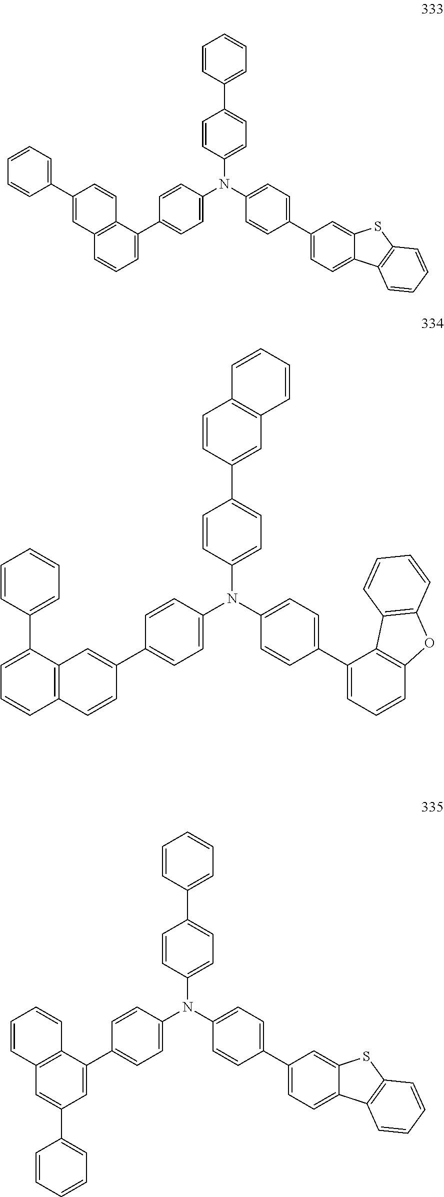 [SMILES: c1ccc(-c2ccc(N(c3ccc(-c4ccc5c(c4)sc4ccccc45)cc3)c3ccc(-c4cccc5cc(-c6ccccc6)ccc45)cc3)cc2)cc1 ; c1ccc(-c2cccc3ccc(-c4ccc(N(c5ccc(-c6ccc7ccccc7c6)cc5)c5ccc(-c6cccc7oc8ccccc8c67)cc5)cc4)cc23)cc1 ; c1ccc(-c2ccc(N(c3ccc(-c4ccc5c(c4)sc4ccccc45)cc3)c3ccc(-c4cc(-c5ccccc5)cc5ccccc45)cc3)cc2)cc1] 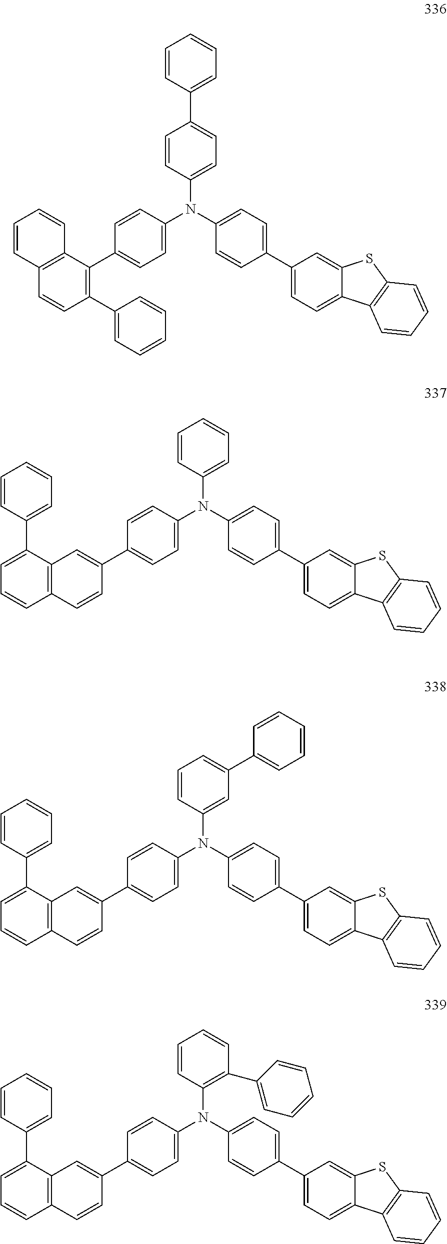 [SMILES: c1ccc(-c2ccc(N(c3ccc(-c4ccc5c(c4)sc4ccccc45)cc3)c3ccc(-c4c(-c5ccccc5)ccc5ccccc45)cc3)cc2)cc1 ; c1ccc(-c2cccc3ccc(-c4ccc(N(c5ccccc5)c5ccc(-c6ccc7c(c6)sc6ccccc67)cc5)cc4)cc23)cc1 ; c1ccc(-c2cccc(N(c3ccc(-c4ccc5c(c4)sc4ccccc45)cc3)c3ccc(-c4ccc5cccc(-c6ccccc6)c5c4)cc3)c2)cc1 ; c1ccc(-c2ccccc2N(c2ccc(-c3ccc4c(c3)sc3ccccc34)cc2)c2ccc(-c3ccc4cccc(-c5ccccc5)c4c3)cc2)cc1] 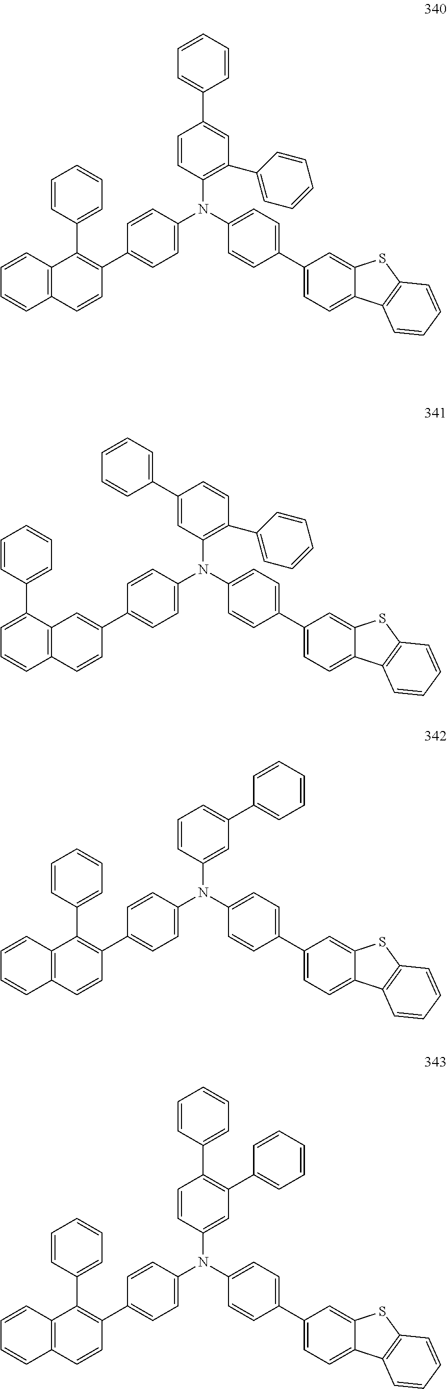 [SMILES: c1ccc(-c2ccc(N(c3ccc(-c4ccc5c(c4)sc4ccccc45)cc3)c3ccc(-c4ccc5ccccc5c4-c4ccccc4)cc3)c(-c3ccccc3)c2)cc1 ; c1ccc(-c2ccc(-c3ccccc3)c(N(c3ccc(-c4ccc5c(c4)sc4ccccc45)cc3)c3ccc(-c4ccc5cccc(-c6ccccc6)c5c4)cc3)c2)cc1 ; c1ccc(-c2cccc(N(c3ccc(-c4ccc5c(c4)sc4ccccc45)cc3)c3ccc(-c4ccc5ccccc5c4-c4ccccc4)cc3)c2)cc1 ; c1ccc(-c2ccc(N(c3ccc(-c4ccc5c(c4)sc4ccccc45)cc3)c3ccc(-c4ccc5ccccc5c4-c4ccccc4)cc3)cc2-c2ccccc2)cc1] 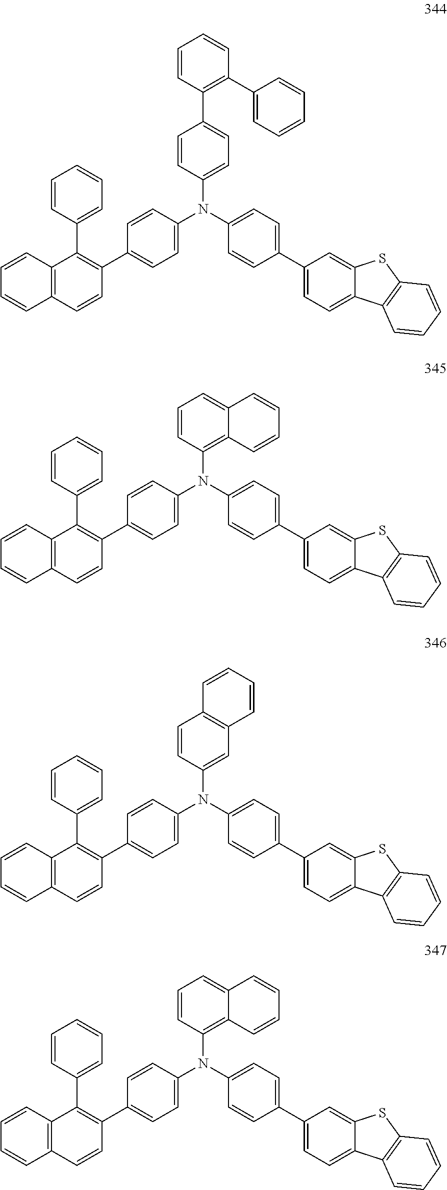 [SMILES: c1ccc(-c2ccccc2-c2ccc(N(c3ccc(-c4ccc5c(c4)sc4ccccc45)cc3)c3ccc(-c4ccc5ccccc5c4-c4ccccc4)cc3)cc2)cc1 ; c1ccc(-c2c(-c3ccc(N(c4ccc(-c5ccc6c(c5)sc5ccccc56)cc4)c4cccc5ccccc45)cc3)ccc3ccccc23)cc1 ; c1ccc(-c2c(-c3ccc(N(c4ccc(-c5ccc6c(c5)sc5ccccc56)cc4)c4ccc5ccccc5c4)cc3)ccc3ccccc23)cc1 ; c1ccc(-c2c(-c3ccc(N(c4ccc(-c5ccc6c(c5)sc5ccccc56)cc4)c4cccc5ccccc45)cc3)ccc3ccccc23)cc1] 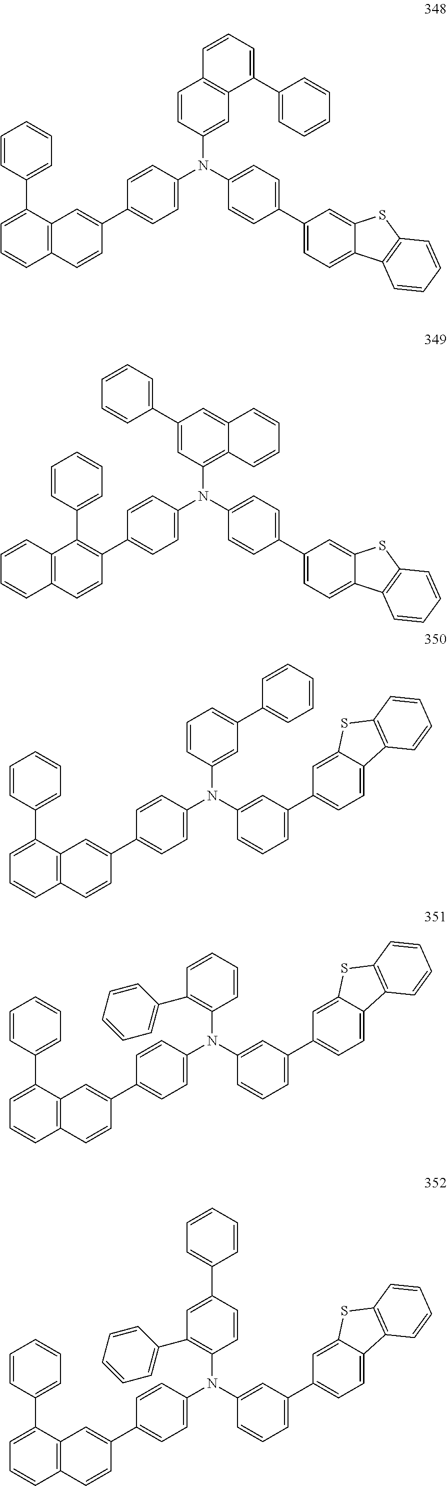 [SMILES: c1ccc(-c2cccc3ccc(-c4ccc(N(c5ccc(-c6ccc7c(c6)sc6ccccc67)cc5)c5ccc6cccc(-c7ccccc7)c6c5)cc4)cc23)cc1 ; c1ccc(-c2cc(N(c3ccc(-c4ccc5c(c4)sc4ccccc45)cc3)c3ccc(-c4ccc5ccccc5c4-c4ccccc4)cc3)c3ccccc3c2)cc1 ; c1ccc(-c2cccc(N(c3ccc(-c4ccc5cccc(-c6ccccc6)c5c4)cc3)c3cccc(-c4ccc5c(c4)sc4ccccc45)c3)c2)cc1 ; c1ccc(-c2ccccc2N(c2ccc(-c3ccc4cccc(-c5ccccc5)c4c3)cc2)c2cccc(-c3ccc4c(c3)sc3ccccc34)c2)cc1 ; c1ccc(-c2ccc(N(c3ccc(-c4ccc5cccc(-c6ccccc6)c5c4)cc3)c3cccc(-c4ccc5c(c4)sc4ccccc45)c3)c(-c3ccccc3)c2)cc1] 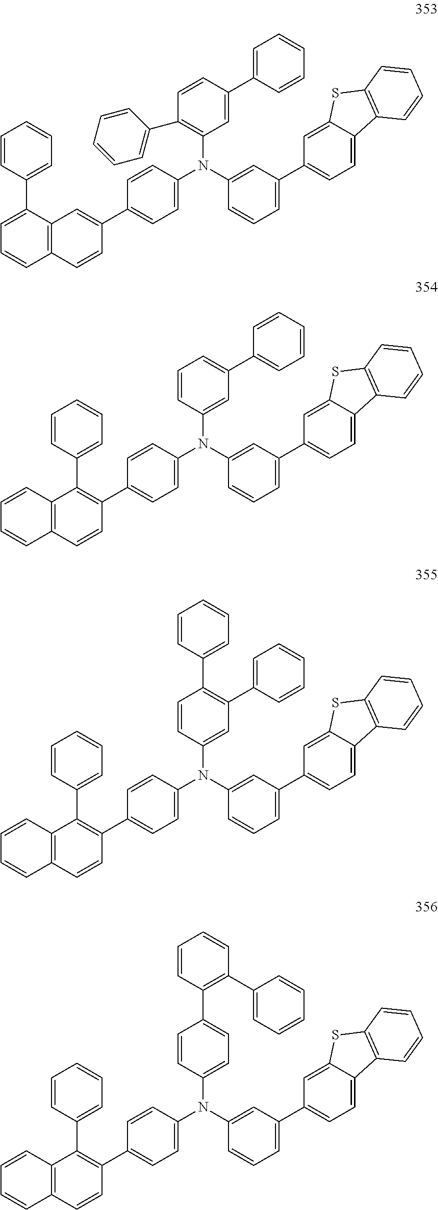 [SMILES: c1ccc(-c2ccc(-c3ccccc3)c(N(c3ccc(-c4ccc5cccc(-c6ccccc6)c5c4)cc3)c3cccc(-c4ccc5c(c4)sc4ccccc45)c3)c2)cc1 ; c1ccc(-c2cccc(N(c3ccc(-c4ccc5ccccc5c4-c4ccccc4)cc3)c3cccc(-c4ccc5c(c4)sc4ccccc45)c3)c2)cc1 ; c1ccc(-c2ccc(N(c3ccc(-c4ccc5ccccc5c4-c4ccccc4)cc3)c3cccc(-c4ccc5c(c4)sc4ccccc45)c3)cc2-c2ccccc2)cc1 ; c1ccc(-c2ccccc2-c2ccc(N(c3ccc(-c4ccc5ccccc5c4-c4ccccc4)cc3)c3cccc(-c4ccc5c(c4)sc4ccccc45)c3)cc2)cc1] 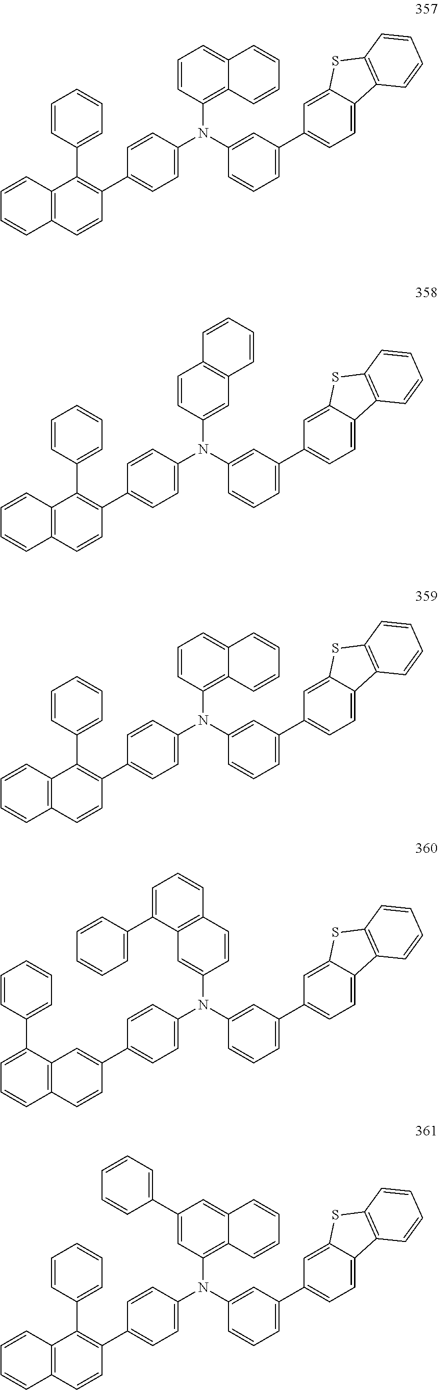 [SMILES: c1ccc(-c2c(-c3ccc(N(c4cccc(-c5ccc6c(c5)sc5ccccc56)c4)c4cccc5ccccc45)cc3)ccc3ccccc23)cc1 ; c1ccc(-c2c(-c3ccc(N(c4cccc(-c5ccc6c(c5)sc5ccccc56)c4)c4ccc5ccccc5c4)cc3)ccc3ccccc23)cc1 ; c1ccc(-c2c(-c3ccc(N(c4cccc(-c5ccc6c(c5)sc5ccccc56)c4)c4cccc5ccccc45)cc3)ccc3ccccc23)cc1 ; c1ccc(-c2cccc3ccc(-c4ccc(N(c5cccc(-c6ccc7c(c6)sc6ccccc67)c5)c5ccc6cccc(-c7ccccc7)c6c5)cc4)cc23)cc1 ; c1ccc(-c2cc(N(c3ccc(-c4ccc5ccccc5c4-c4ccccc4)cc3)c3cccc(-c4ccc5c(c4)sc4ccccc45)c3)c3ccccc3c2)cc1] 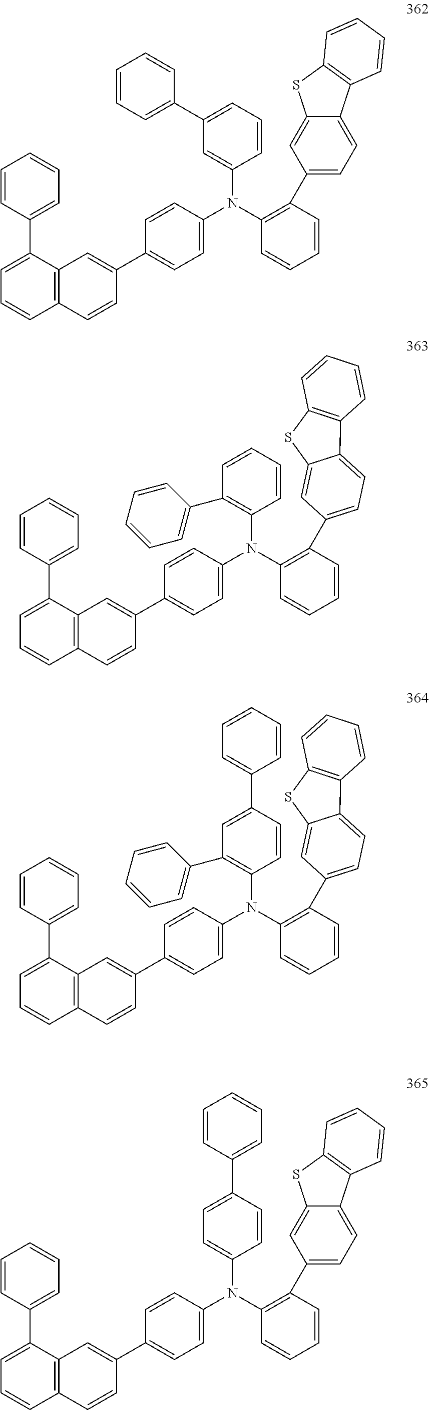 [SMILES: c1ccc(-c2cccc(N(c3ccc(-c4ccc5cccc(-c6ccccc6)c5c4)cc3)c3ccccc3-c3ccc4c(c3)sc3ccccc34)c2)cc1 ; c1ccc(-c2ccccc2N(c2ccc(-c3ccc4cccc(-c5ccccc5)c4c3)cc2)c2ccccc2-c2ccc3c(c2)sc2ccccc23)cc1 ; c1ccc(-c2ccc(N(c3ccc(-c4ccc5cccc(-c6ccccc6)c5c4)cc3)c3ccccc3-c3ccc4c(c3)sc3ccccc34)c(-c3ccccc3)c2)cc1 ; c1ccc(-c2ccc(N(c3ccc(-c4ccc5cccc(-c6ccccc6)c5c4)cc3)c3ccccc3-c3ccc4c(c3)sc3ccccc34)cc2)cc1] 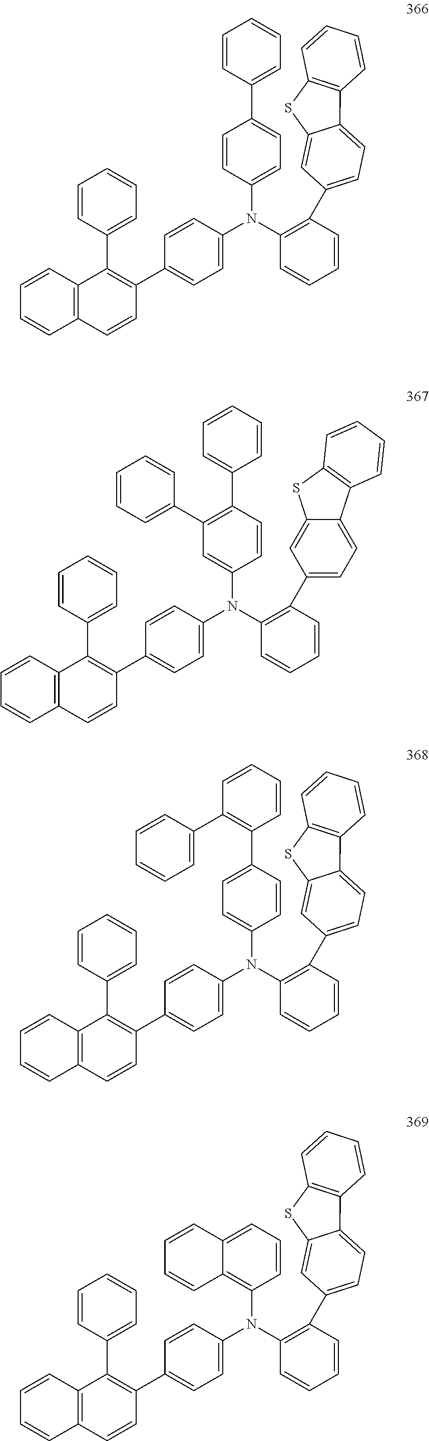 [SMILES: c1ccc(-c2ccc(N(c3ccc(-c4ccc5ccccc5c4-c4ccccc4)cc3)c3ccccc3-c3ccc4c(c3)sc3ccccc34)cc2)cc1 ; c1ccc(-c2ccc(N(c3ccc(-c4ccc5ccccc5c4-c4ccccc4)cc3)c3ccccc3-c3ccc4c(c3)sc3ccccc34)cc2-c2ccccc2)cc1 ; c1ccc(-c2ccccc2-c2ccc(N(c3ccc(-c4ccc5ccccc5c4-c4ccccc4)cc3)c3ccccc3-c3ccc4c(c3)sc3ccccc34)cc2)cc1 ; c1ccc(-c2c(-c3ccc(N(c4ccccc4-c4ccc5c(c4)sc4ccccc45)c4cccc5ccccc45)cc3)ccc3ccccc23)cc1] 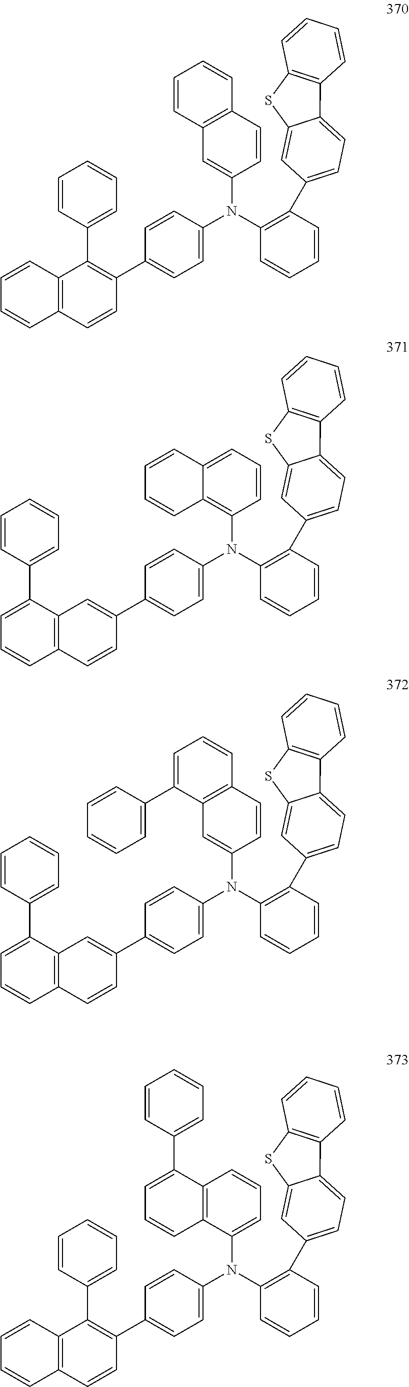 [SMILES: c1ccc(-c2c(-c3ccc(N(c4ccc5ccccc5c4)c4ccccc4-c4ccc5c(c4)sc4ccccc45)cc3)ccc3ccccc23)cc1 ; c1ccc(-c2cccc3ccc(-c4ccc(N(c5ccccc5-c5ccc6c(c5)sc5ccccc56)c5cccc6ccccc56)cc4)cc23)cc1 ; c1ccc(-c2cccc3ccc(-c4ccc(N(c5ccc6cccc(-c7ccccc7)c6c5)c5ccccc5-c5ccc6c(c5)sc5ccccc56)cc4)cc23)cc1 ; c1ccc(-c2c(-c3ccc(N(c4ccccc4-c4ccc5c(c4)sc4ccccc45)c4cccc5c(-c6ccccc6)cccc45)cc3)ccc3ccccc23)cc1] 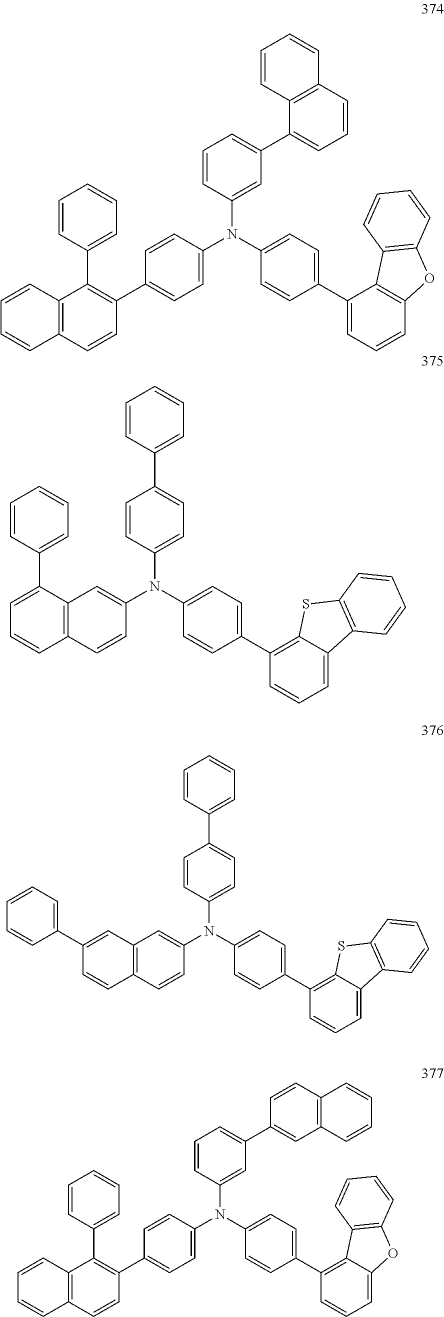 [SMILES: c1ccc(-c2c(-c3ccc(N(c4ccc(-c5cccc6oc7ccccc7c56)cc4)c4cccc(-c5cccc6ccccc56)c4)cc3)ccc3ccccc23)cc1 ; c1ccc(-c2ccc(N(c3ccc(-c4cccc5c4sc4ccccc45)cc3)c3ccc4cccc(-c5ccccc5)c4c3)cc2)cc1 ; c1ccc(-c2ccc(N(c3ccc(-c4cccc5c4sc4ccccc45)cc3)c3ccc4ccc(-c5ccccc5)cc4c3)cc2)cc1 ; c1ccc(-c2c(-c3ccc(N(c4ccc(-c5cccc6oc7ccccc7c56)cc4)c4cccc(-c5ccc6ccccc6c5)c4)cc3)ccc3ccccc23)cc1] 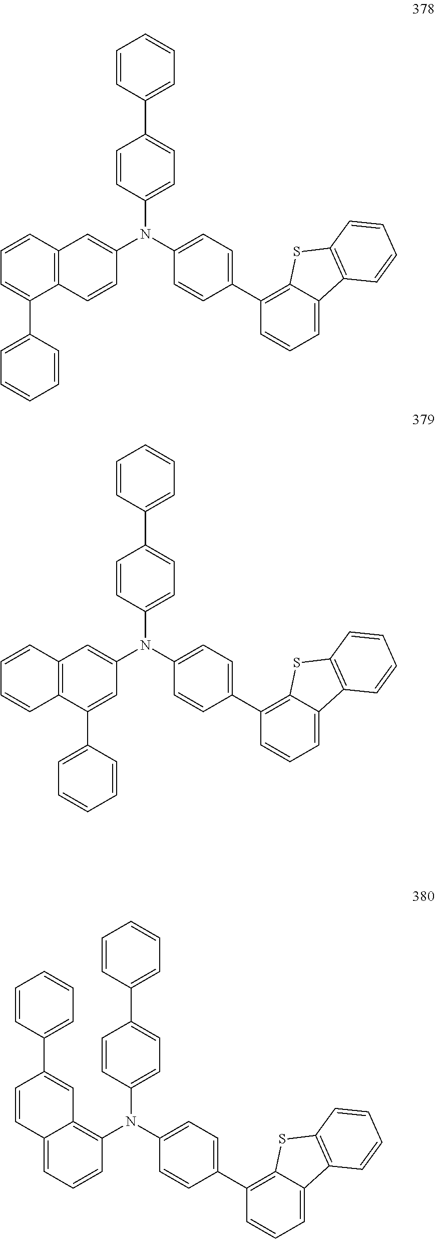 [SMILES: c1ccc(-c2ccc(N(c3ccc(-c4cccc5c4sc4ccccc45)cc3)c3ccc4c(-c5ccccc5)cccc4c3)cc2)cc1 ; c1ccc(-c2ccc(N(c3ccc(-c4cccc5c4sc4ccccc45)cc3)c3cc(-c4ccccc4)c4ccccc4c3)cc2)cc1 ; c1ccc(-c2ccc(N(c3ccc(-c4cccc5c4sc4ccccc45)cc3)c3cccc4ccc(-c5ccccc5)cc34)cc2)cc1] 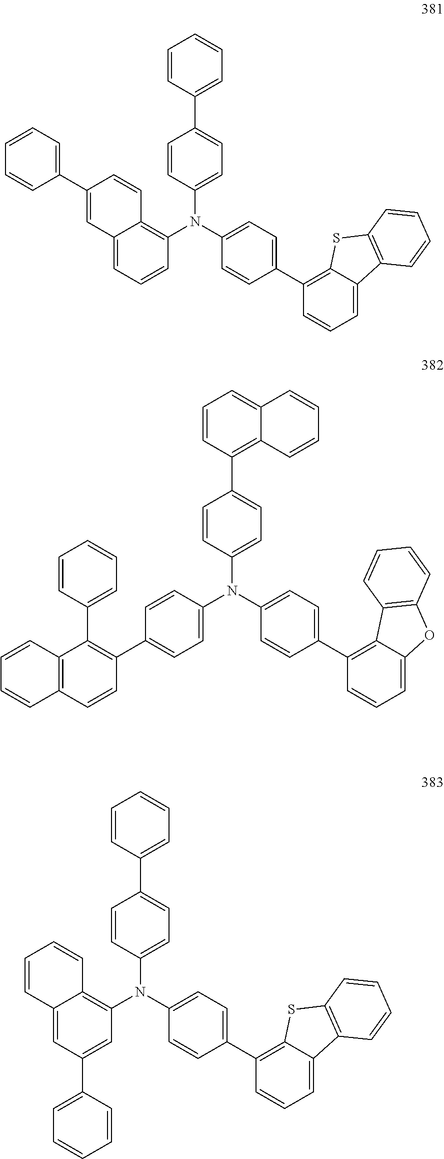 [SMILES: c1ccc(-c2ccc(N(c3ccc(-c4cccc5c4sc4ccccc45)cc3)c3cccc4cc(-c5ccccc5)ccc34)cc2)cc1 ; c1ccc(-c2c(-c3ccc(N(c4ccc(-c5cccc6ccccc56)cc4)c4ccc(-c5cccc6oc7ccccc7c56)cc4)cc3)ccc3ccccc23)cc1 ; c1ccc(-c2ccc(N(c3ccc(-c4cccc5c4sc4ccccc45)cc3)c3cc(-c4ccccc4)cc4ccccc34)cc2)cc1] 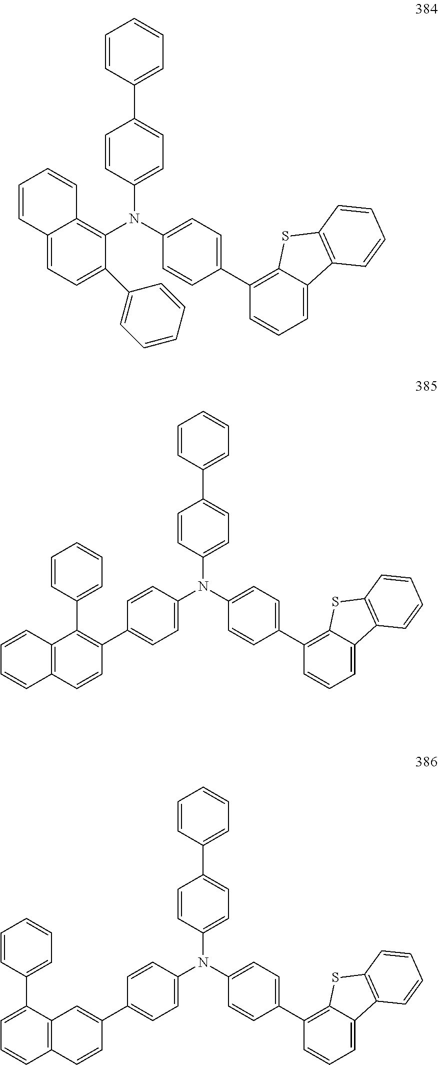 [SMILES: c1ccc(-c2ccc(N(c3ccc(-c4cccc5c4sc4ccccc45)cc3)c3c(-c4ccccc4)ccc4ccccc34)cc2)cc1 ; c1ccc(-c2ccc(N(c3ccc(-c4ccc5ccccc5c4-c4ccccc4)cc3)c3ccc(-c4cccc5c4sc4ccccc45)cc3)cc2)cc1 ; c1ccc(-c2ccc(N(c3ccc(-c4ccc5cccc(-c6ccccc6)c5c4)cc3)c3ccc(-c4cccc5c4sc4ccccc45)cc3)cc2)cc1] 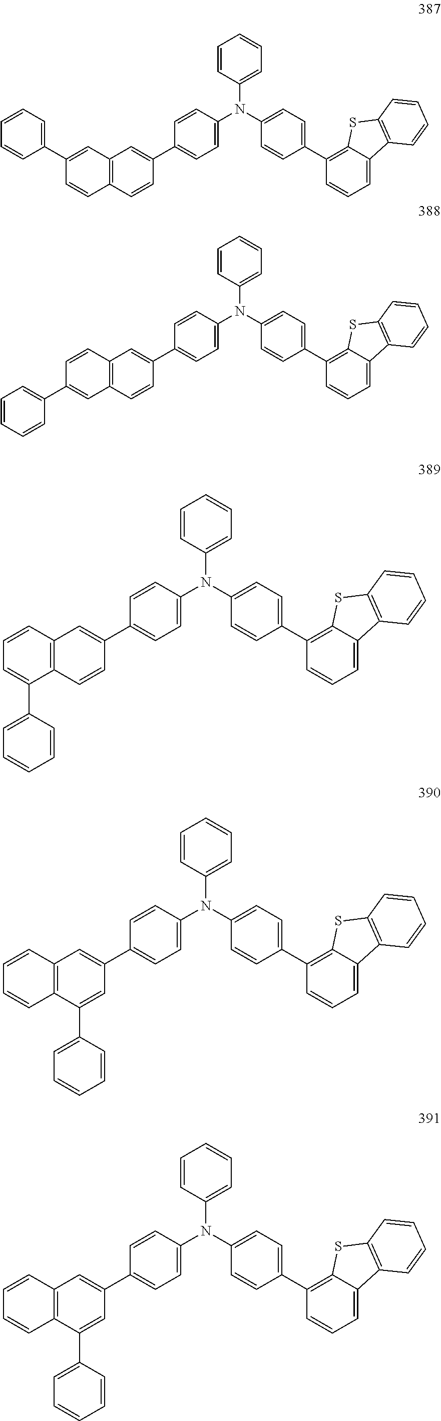 [SMILES: c1ccc(-c2ccc3ccc(-c4ccc(N(c5ccccc5)c5ccc(-c6cccc7c6sc6ccccc67)cc5)cc4)cc3c2)cc1 ; c1ccc(-c2ccc3cc(-c4ccc(N(c5ccccc5)c5ccc(-c6cccc7c6sc6ccccc67)cc5)cc4)ccc3c2)cc1 ; c1ccc(-c2cccc3cc(-c4ccc(N(c5ccccc5)c5ccc(-c6cccc7c6sc6ccccc67)cc5)cc4)ccc23)cc1 ; c1ccc(-c2cc(-c3ccc(N(c4ccccc4)c4ccc(-c5cccc6c5sc5ccccc56)cc4)cc3)cc3ccccc23)cc1 ; c1ccc(-c2cc(-c3ccc(N(c4ccccc4)c4ccc(-c5cccc6c5sc5ccccc56)cc4)cc3)cc3ccccc23)cc1] 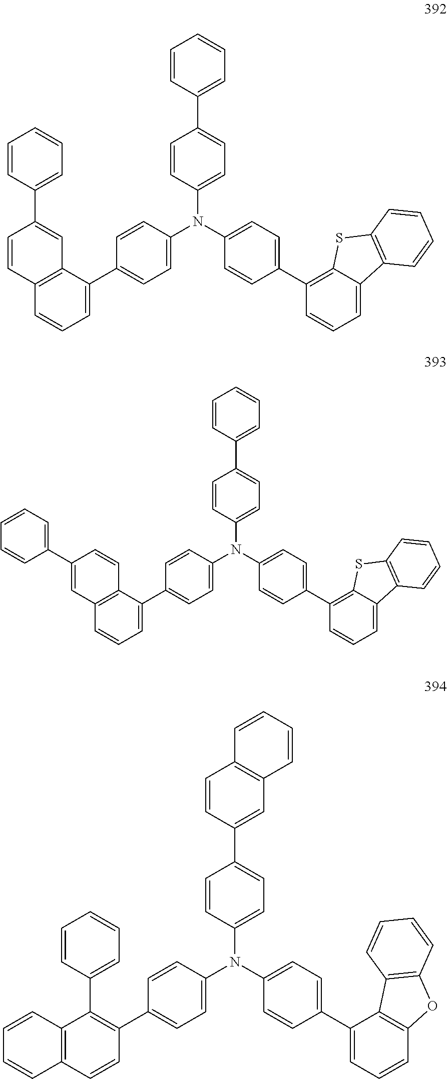 [SMILES: c1ccc(-c2ccc(N(c3ccc(-c4cccc5ccc(-c6ccccc6)cc45)cc3)c3ccc(-c4cccc5c4sc4ccccc45)cc3)cc2)cc1 ; c1ccc(-c2ccc(N(c3ccc(-c4cccc5cc(-c6ccccc6)ccc45)cc3)c3ccc(-c4cccc5c4sc4ccccc45)cc3)cc2)cc1 ; c1ccc(-c2c(-c3ccc(N(c4ccc(-c5ccc6ccccc6c5)cc4)c4ccc(-c5cccc6oc7ccccc7c56)cc4)cc3)ccc3ccccc23)cc1] 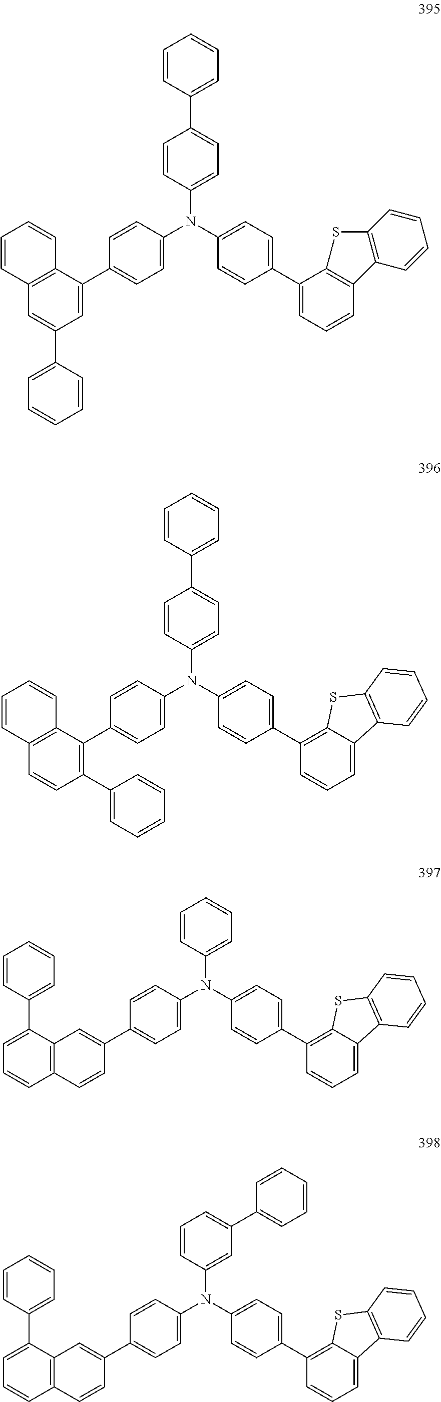 [SMILES: c1ccc(-c2ccc(N(c3ccc(-c4cc(-c5ccccc5)cc5ccccc45)cc3)c3ccc(-c4cccc5c4sc4ccccc45)cc3)cc2)cc1 ; c1ccc(-c2ccc(N(c3ccc(-c4c(-c5ccccc5)ccc5ccccc45)cc3)c3ccc(-c4cccc5c4sc4ccccc45)cc3)cc2)cc1 ; c1ccc(-c2cccc3ccc(-c4ccc(N(c5ccccc5)c5ccc(-c6cccc7c6sc6ccccc67)cc5)cc4)cc23)cc1 ; c1ccc(-c2cccc(N(c3ccc(-c4ccc5cccc(-c6ccccc6)c5c4)cc3)c3ccc(-c4cccc5c4sc4ccccc45)cc3)c2)cc1] 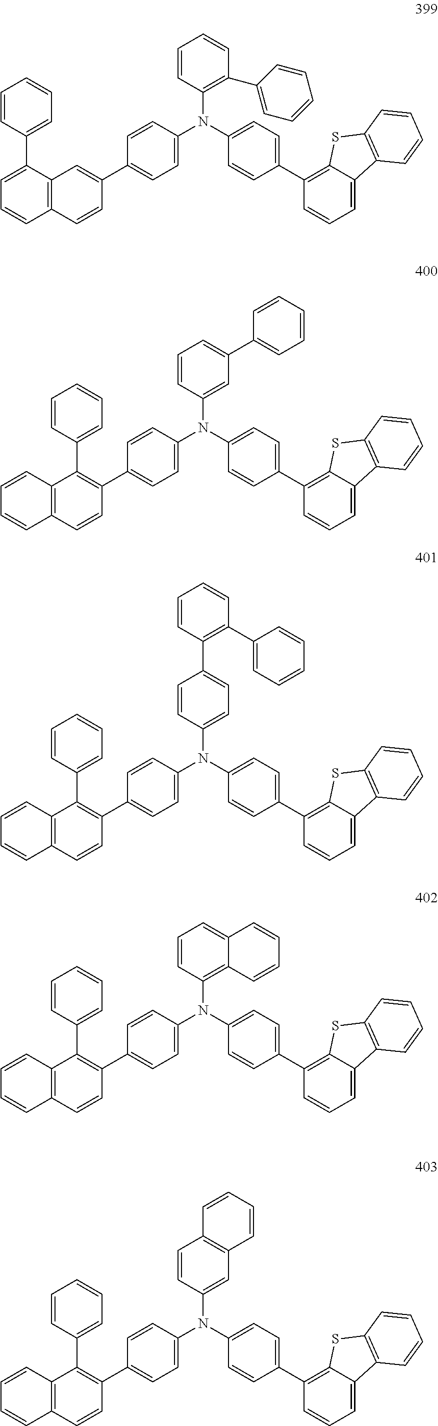 [SMILES: c1ccc(-c2ccccc2N(c2ccc(-c3ccc4cccc(-c5ccccc5)c4c3)cc2)c2ccc(-c3cccc4c3sc3ccccc34)cc2)cc1 ; c1ccc(-c2cccc(N(c3ccc(-c4ccc5ccccc5c4-c4ccccc4)cc3)c3ccc(-c4cccc5c4sc4ccccc45)cc3)c2)cc1 ; c1ccc(-c2ccccc2-c2ccc(N(c3ccc(-c4ccc5ccccc5c4-c4ccccc4)cc3)c3ccc(-c4cccc5c4sc4ccccc45)cc3)cc2)cc1 ; c1ccc(-c2c(-c3ccc(N(c4ccc(-c5cccc6c5sc5ccccc56)cc4)c4cccc5ccccc45)cc3)ccc3ccccc23)cc1 ; c1ccc(-c2c(-c3ccc(N(c4ccc(-c5cccc6c5sc5ccccc56)cc4)c4ccc5ccccc5c4)cc3)ccc3ccccc23)cc1] 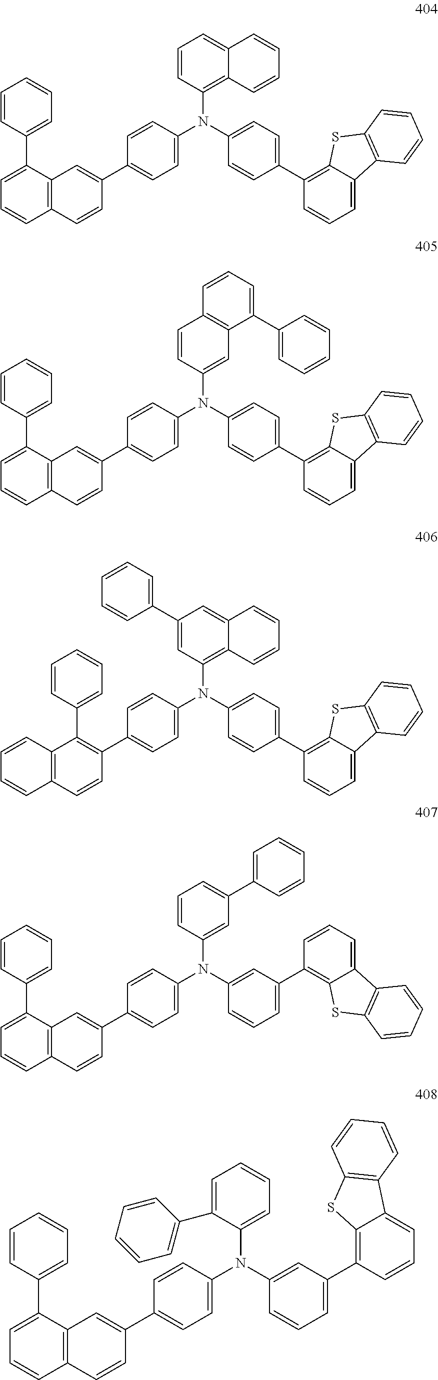 [SMILES: c1ccc(-c2cccc3ccc(-c4ccc(N(c5ccc(-c6cccc7c6sc6ccccc67)cc5)c5cccc6ccccc56)cc4)cc23)cc1 ; c1ccc(-c2cccc3ccc(-c4ccc(N(c5ccc(-c6cccc7c6sc6ccccc67)cc5)c5ccc6cccc(-c7ccccc7)c6c5)cc4)cc23)cc1 ; c1ccc(-c2cc(N(c3ccc(-c4ccc5ccccc5c4-c4ccccc4)cc3)c3ccc(-c4cccc5c4sc4ccccc45)cc3)c3ccccc3c2)cc1 ; c1ccc(-c2cccc(N(c3ccc(-c4ccc5cccc(-c6ccccc6)c5c4)cc3)c3cccc(-c4cccc5c4sc4ccccc45)c3)c2)cc1 ; c1ccc(-c2ccccc2N(c2ccc(-c3ccc4cccc(-c5ccccc5)c4c3)cc2)c2cccc(-c3cccc4c3sc3ccccc34)c2)cc1] 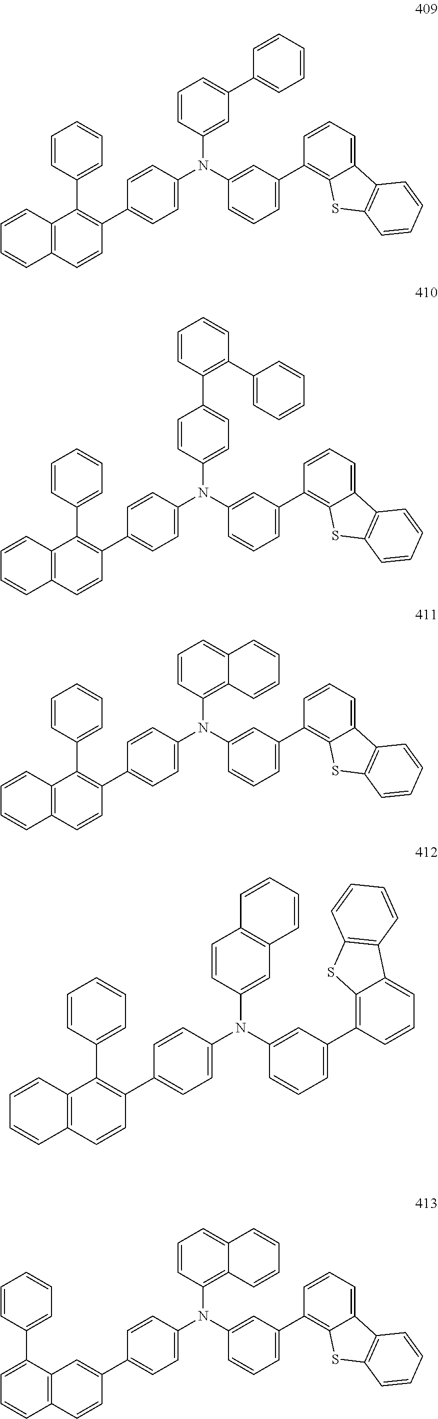 [SMILES: c1ccc(-c2cccc(N(c3ccc(-c4ccc5ccccc5c4-c4ccccc4)cc3)c3cccc(-c4cccc5c4sc4ccccc45)c3)c2)cc1 ; c1ccc(-c2ccccc2-c2ccc(N(c3ccc(-c4ccc5ccccc5c4-c4ccccc4)cc3)c3cccc(-c4cccc5c4sc4ccccc45)c3)cc2)cc1 ; c1ccc(-c2c(-c3ccc(N(c4cccc(-c5cccc6c5sc5ccccc56)c4)c4cccc5ccccc45)cc3)ccc3ccccc23)cc1 ; c1ccc(-c2c(-c3ccc(N(c4cccc(-c5cccc6c5sc5ccccc56)c4)c4ccc5ccccc5c4)cc3)ccc3ccccc23)cc1 ; c1ccc(-c2cccc3ccc(-c4ccc(N(c5cccc(-c6cccc7c6sc6ccccc67)c5)c5cccc6ccccc56)cc4)cc23)cc1] 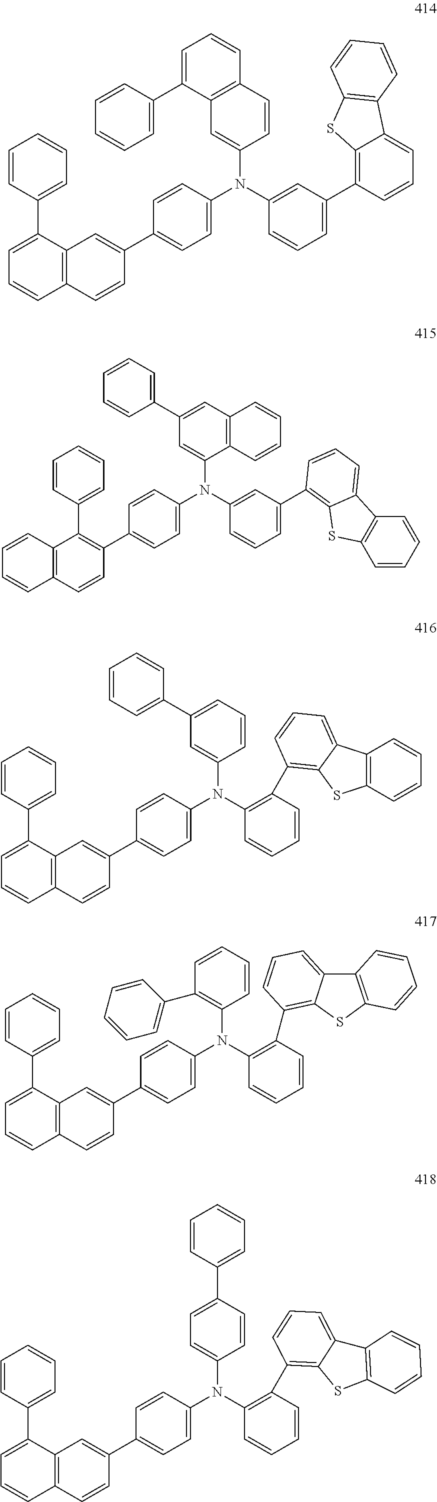 [SMILES: c1ccc(-c2cccc3ccc(-c4ccc(N(c5cccc(-c6cccc7c6sc6ccccc67)c5)c5ccc6cccc(-c7ccccc7)c6c5)cc4)cc23)cc1 ; c1ccc(-c2cc(N(c3ccc(-c4ccc5ccccc5c4-c4ccccc4)cc3)c3cccc(-c4cccc5c4sc4ccccc45)c3)c3ccccc3c2)cc1 ; c1ccc(-c2cccc(N(c3ccc(-c4ccc5cccc(-c6ccccc6)c5c4)cc3)c3ccccc3-c3cccc4c3sc3ccccc34)c2)cc1 ; c1ccc(-c2ccccc2N(c2ccc(-c3ccc4cccc(-c5ccccc5)c4c3)cc2)c2ccccc2-c2cccc3c2sc2ccccc23)cc1 ; c1ccc(-c2ccc(N(c3ccc(-c4ccc5cccc(-c6ccccc6)c5c4)cc3)c3ccccc3-c3cccc4c3sc3ccccc34)cc2)cc1] 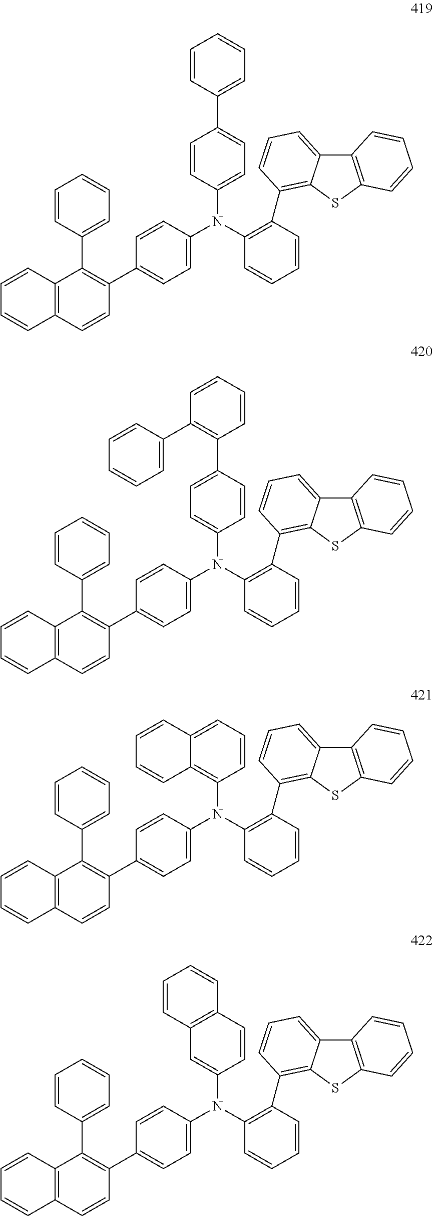 [SMILES: c1ccc(-c2ccc(N(c3ccc(-c4ccc5ccccc5c4-c4ccccc4)cc3)c3ccccc3-c3cccc4c3sc3ccccc34)cc2)cc1 ; c1ccc(-c2ccccc2-c2ccc(N(c3ccc(-c4ccc5ccccc5c4-c4ccccc4)cc3)c3ccccc3-c3cccc4c3sc3ccccc34)cc2)cc1 ; c1ccc(-c2c(-c3ccc(N(c4ccccc4-c4cccc5c4sc4ccccc45)c4cccc5ccccc45)cc3)ccc3ccccc23)cc1 ; c1ccc(-c2c(-c3ccc(N(c4ccc5ccccc5c4)c4ccccc4-c4cccc5c4sc4ccccc45)cc3)ccc3ccccc23)cc1] 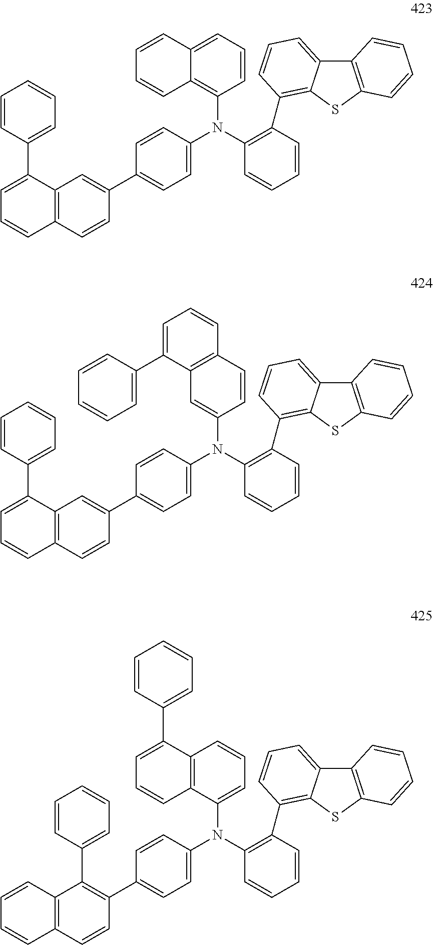 [SMILES: c1ccc(-c2cccc3ccc(-c4ccc(N(c5ccccc5-c5cccc6c5sc5ccccc56)c5cccc6ccccc56)cc4)cc23)cc1 ; c1ccc(-c2cccc3ccc(-c4ccc(N(c5ccc6cccc(-c7ccccc7)c6c5)c5ccccc5-c5cccc6c5sc5ccccc56)cc4)cc23)cc1 ; c1ccc(-c2c(-c3ccc(N(c4ccccc4-c4cccc5c4sc4ccccc45)c4cccc5c(-c6ccccc6)cccc45)cc3)ccc3ccccc23)cc1]
wherein in Compound Group 1, D is a deuterium atom.

18. An electronic device comprising a display module including a plurality of light emitting elements,
wherein at least one of the plurality of light emitting elements includes a first electrode, a second electrode disposed on the first electrode, an emission layer between the first electrode and the second electrode, and a hole transport region between the first electrode and the emission layer, the hole transport region including an amine compound represented by Formula 1: 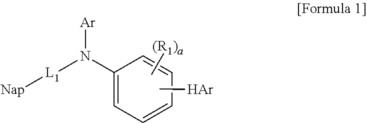
wherein in Formula 1,
L₁ is a direct linkage, a substituted or unsubstituted arylene group having 6 to 30 ring-forming carbon atoms excluding a substituted or unsubstituted naphthyl group, or a substituted or unsubstituted heteroarylene group having 5 to 30 ring-forming carbon atoms,
Ar is a substituted or unsubstituted phenyl group without including an aryl group or a heteroaryl group having 16 or more ring-forming carbon atoms as a substituent, or a substituted or unsubstituted naphthyl group,
a is an integer of 0 to 4,
R₁ is a hydrogen atom, a deuterium atom, a halogen atom, a substituted or unsubstituted alkyl group having 1 to 15 carbon atoms, or a substituted or unsubstituted phenyl group, and
HAr is represented by Formula 2, and Nap is represented by Formula 3 or Formula 4: 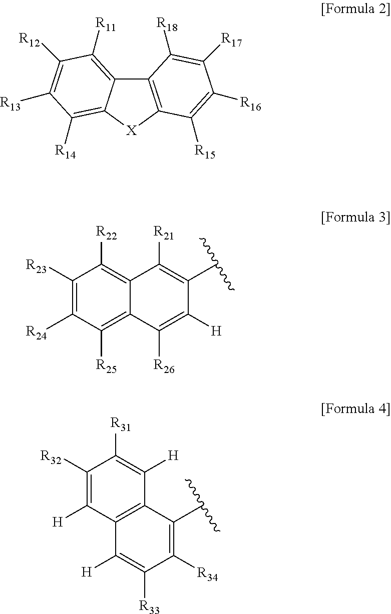
wherein in Formula 2,
X is O or S,
any one of R₁₁ to R₁₄ is a position linked to Formula 1, and the other positions of R₁₁ to R₁₄ excluding the linked position, and R₁₅ to R₁₈ are each independently a hydrogen atom, a deuterium atom, a halogen atom, a substituted or unsubstituted alkyl group having 1 to 15 carbon atoms, or a substituted or unsubstituted phenyl group,
when X is O and when linked to Formula 1 at the R₁₁ position, the R₁₁ position is para to a nitrogen atom of Formula 1,
when linked to Formula 1 at the R₁₂ position, a case in which R₁₅ is a substituted or unsubstituted phenyl group is excluded,
when linked to Formula 1 at the R₁₄ position, Ar of Formula 1 either includes a total of one aryl or heteroaryl group as a substituent or does not include any,
when linked to Formula 1 at the R₁₄ position and when L¹ is a direct linkage, a case in which R₁ is a phenyl group is excluded,
wherein in Formula 3, any one of R₂₁ to R₂₆ is a substituted or unsubstituted phenyl group, and the others are each independently a substituted or unsubstituted alkyl group having 1 to 15 carbon atoms, a halogen atom, a hydrogen atom, or a deuterium atom,
when linked to Formula 1 at the R₁₄ position of Formula 2 and when L¹ is a direct linkage, cases in which R₂₁ or R₂₄ is a substituted or unsubstituted phenyl group are excluded,
wherein in Formula 4, any one of R₃₁ to R₃₄ is a substituted or unsubstituted phenyl group, and the others are each independently a substituted or unsubstituted alkyl group having 1 to 15 carbon atoms, a halogen atom, a hydrogen atom, or a deuterium atom, and
when linked to Formula 1 at the R₁₄ position of Formula 2, a case in which R₃₄ is a substituted or unsubstituted phenyl group is excluded.

19. The electronic device of claim 18, wherein at least one of the light emitting elements emits blue light.

20. The electronic device of claim 18, further comprising at least one of a processor, a memory, or a power module.
